(19) United States
(12) Patent Application Publication
(10) Pub. No.: US 2020/0131176 A1
(43) Pub. Date: Apr. 30, 2020

(54) SELECTIVE INHIBITORS OF CLINICALLY IMPORTANT MUTANTS OF THE EGFR TYROSINE KINASE

(71) Applicant: CS Pharmatech Limited, Grand Cayman (KR)

(72) Inventors: Yuntao SONG, Palo Alto, CA (US); Alexander James BRIDGES, Saline, MI (US); Xiaoqi CHEN, Palo Alto, CA (US)

(21) Appl. No.: 16/628,831

(22) PCT Filed: Jul. 5, 2018
(86) PCT No.: PCT/US2018/040904

§ 371 Date: Jan. 6, 2020

Related U.S. Application Data
(60) Provisional application No. 62/528,697, filed on Jul. 5, 2017.

(51) Int. Cl. C07D 471/04; C07D 403/10; C07D 495/04; C07D 403/04; A61P 35/00

(52) U.S. Cl. CPC C07D 471/04; C07D 403/10; A61P 35/00; C07D 403/04; C07D 495/04

(57) ABSTRACT

The present invention provides compounds of Formula (I) or a subgeneric structure or species thereof, or a pharmaceutically acceptable salt, ester, solvate, and/or prodrug thereof, and methods and compositions for treating or ameliorating abnormal cell proliferative disorders, such as cancer, wherein A, R², R³, R¹⁰, E¹, E², E³, Y, and Z are as defined herein.

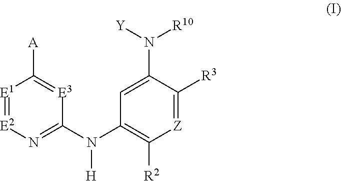

### CROSS-REFERENCE TO RELATED APPLICATIONS

This application claims the benefit of U.S. Provisional Patent Application No. 62/528,697 filed Jul. 5, 2017, the disclosures of which are hereby incorporated by reference in its entirety for all purposes.

### FIELD OF THE INVENTION

The present invention relates to compounds of formula (I) or subgeneric structures or species thereof or their pharmaceutically acceptable salts ester, solvate, and/or prodrug thereof, and pharmaceutical compositions comprising such compounds or a pharmaceutically acceptable salt ester, solvate, and/or prodrug thereof. The compounds and salts of the present invention inhibit kinases, especially the epidermal growth factor receptor EGFR, and particular mutants of it, important in developing resistance to treatment by EGFR inhibitory therapy, and are useful for treating or ameliorating abnormal cell proliferative disorders, such as cancer.

### BACKGROUND OF THE INVENTION

The current invention pertains to biarylamino compounds which are useful as highly selective inhibitors of certain protein tyrosine kinases, PTKs, which are one of the sub-classes of the protein kinases, PKs. PKs are very important signaling entities in intracellular communication, where they modify many proteins by catalyzing the transfer of a phosphate group from ATP acting as a phosphodonor to a phenolic hydroxyl on a tyrosine side chain of the protein. Frequently, the tyrosine kinases are incorporated into the intracellular domain of a very large transmembrane protein, which has a cognate ligand binding domain in the extracellular domain, whereby ligand binding activates the tyrosine kinase intracellularly. Such molecules are receptor tyrosine kinases (RTKs).

Structurally, the kinases are quite well understood. There is a kinase domain, which may be the whole protein, or only one domain of a much larger modular protein, and this domain has a basic conserved structure of about 35 kD, consisting of two lobes, the N-terminal one being mainly made up of β-sheets, and the larger C-terminal domain mainly of α-helices. There is a deep cleft between the two lobes which binds both ATP and the substrate. The substrate binding domain is quite large, and rather variable, and is used to discriminate between different protein substrates, and maintain specificity of phosphorylation. This specificity can be very variable, with some enzymes such as MEK having only one known substrate, and others being able to phosphorylate hundreds of distinct hydroxyls in proteins.

Phosphorylation frequently changes the conformation of the modified protein, often converting enzymes from an inactive form to an active form, or vice versa, or causing the protein to associate closely with specific binding partners, or perhaps dissociate from them, leading to changes in cellular localization, or assembly, or disassembly, of functioning multi-protein complexes. Many of the transducers of signals into cells, and from the cell surface into the nucleus are either PKs, or controlled by PKs, especially RTKs. Because of this, inhibitors of the kinase activity of PKs can have very drastic effects on cellular signaling, damping down both normal responses to external signals, and inappropriate overresponses, usually caused by mutations in or aberrant expression levels of one or more of the signaling molecules themselves. Although such pathways are very widespread in the body, and are involved in one way or another in most bodily functions, and the diseases that can arise from their malfunction, inhibitors of PKs are particularly useful in treating cancer and immunological disorders, both disease classes where over-activity of PKs, especially RTKs, has been widely documented, and where they often play crucial roles in driving the disease process itself.

Kinases have been shown to be very important effectors of many disease processes, especially in cancer. Cellular proliferation is controlled at many different levels by kinases, and, under normal circumstances for cells to proliferate, signals have to be sent from outside the cell, where they bind to receptors and activate the receptors. Many of the important receptors in cell signaling are kinases, especially RTKs, or are directly coupled to kinases which themselves are activated by the activated receptor. Once these kinases have been activated, they in turn activate signaling cascades, which usually involve several further kinases in an amplifying wave of phosphorylation, which lead eventually to the translocation into, and activation of, transcription factors in the nucleus. Activation of the transcription factors engenders proteins being produced which carry out various programs within the cell, including those which start the cell into the proliferative cycle. Usually, once this process has gone on for a number of hours, the newly synthesized proteins will continue the process, without need for further extracellular input. If the proliferative cell cycle is initiated, the first set of proteins synthesized includes both further transcription factors, and their activators to drive later stages of the cell cycle, and effectors, which start the process of duplicating and dividing the cell. Kinases are major controllers of every step in this process. When this process is not controlled properly, and cells can execute the cell cycle without appropriate external control, they become transformed, and can form a tumor, if the immune system fails to eradicate them.

When transformed cells are examined, one of their invariant characteristics is hyperphosphorylation, showing that these cells have an overall surfeit of kinase activity, especially in the absence of any growth factors. Hyperphosphorylation can be caused by a very wide variety of mutations in the cell. For example by the cell inappropriately producing its own ligand for one of the receptor-linked kinases. Or one of these kinases may be heavily overexpressed, due either to a failure to control its expression properly, or to multiple extra copies of the gene being present in the cell. Another very common genetic defect is a mutation in the coding region of the kinase, which leads to a kinase which is constitutively active, and has no need for the appropriate signal to active it. Sometimes the kinase may not be inappropriately active, but a phosphatase, which is supposed to limit its signaling by removing the phosphate from target molecules, is inactivated by mutation or deletion. Examination of both cell culture tumors and isolates from clinical tumors will almost always find defects of this sort in the phosphorylation system of the tumor cells.

In the late 1980s, several small molecule kinase inhibitors were discovered. These molecules almost invariably bind in the catalytic cleft of the kinase, and compete with ATP for its binding site. Thus they are ATP-competitive, and most inhibitors discovered since then fall into this class. However, kinase inhibitors have been occasionally discovered which compete with the protein substrate, substrate-competitive, or more commonly with both ATP and substrate, dual inhibitors, or are neither competitive with receptor nor substrate, non-competitive inhibitors. After allowing for differences in cellular penetration, one finds that there is a very good correlation between the potency of these compounds in isolated kinase enzyme inhibitory assays, and inhibition of the kinase in cells. For many kinases, there is also an excellent correlation between loss of phosphorylation of downstream targets, and inhibition of cellular proliferation. As this correlation has been shown thousands of times, with dozens of different kinases, it is a clear demonstration that aberrant kinase signaling can cause uncontrolled proliferation in transformed cells, and that in many cases, blockade of the over-activated kinase can stop the proliferation. In many cases the kinase inhibitor alone can actually induce apoptosis in the transformed cells, leading to shrinkage of the tumor. This can occur because various genetic lesions in the cell have been detected by the cellular proof-reading system, and as a result several pro-apoptotic mechanisms are usually activated in these cells, but aberrant phosphorylation may well be involved in suppressing the ongoing apoptotic process. Some kinase inhibitors, especially those which target kinases involved late in the cell cycle are intrinsically cytotoxic, as cells interrupted during mitosis tend to apoptose very readily. Although, good proof that these abilities in cells could prevent tumors grown as xenografts in nude mice was initially slow in coming, as the agents improved, it became routine to demonstrate that kinase inhibitors could slow the growth of tumors which express the kinase oncogenes being targeted, and the better agents cause the tumors to regress in size often to the point of immeasurability, and on rare occasions the tumors do not regrow after dosing is stopped, suggesting the animals may have been cured of the tumor. Furthermore, the in vivo efficacy correlates with the cellular and enzymatic activity, after one has correlated for tumor exposure.

Clinical proof was slower in coming, probably partly because clinical tumors are often much more complex than tumors grown under carefully controlled conditions, partly because mice are a lot more biochemically robust than humans, and can tolerate larger relative doses of the drugs, and mainly because it is usually very difficult to know which are the appropriate kinases to inhibit in any given randomly presenting human tumor. However imatinib, a reasonably potent inhibitor of the fusion oncogenic TK BCR-ABL, with truly outstanding pharmacokinetic properties, was approved for chronic myelogenous leukemia (CML) in 2000. This kinase inhibitor provides a very convincing clinical proof of concept for the theory, as about two thirds of CML patients (whose tumors almost by definition contain one of two forms of BCR-ABL) respond very well to treatment, and usually the leukemia cells almost completely disappear from circulation. Surprisingly, mutation around this blockade appears to be very slow, and even after 10 years of treatment the drug is still effective in 80% of patients. This has not proved to be the general case, probably partly because most tumors are found much later in their biological history than are CMLs, and have had much longer to become genetically heterogeneous, and partly because very few tumors are as dependent on one oncogene as CML is on BCR-ABL.

### Epidermal Growth Factor Receptor Tyrosine Kinase Inhibitors

Two 4-anilinoquinazoline inhibitors of the epidermal growth factor RTK (EGFR, erbB-1), gefitinib and erlotinib, were approved for use in lung cancer around 10 years ago. EGFR is one of the most commonly dysregulated kinases seen in solid tumors, with overexpression or mutation being seen often in 50% or more of a tumor type, including non-small cell lung cancer (NSCLC). Despite excellent activity of these inhibitors against a wide variety of xenografts overexpressing EGFR, very limited activity was seen in NSCLC, with only about 10% of patients responding to the drug, and the average response only lasting a year or so, although occasionally a much more durable responder is found. Surprisingly, in other tumor types known to overexpress EGFR, especially colorectal cancer (CRC) no meaningful activity was demonstrated, although the anti-EGFR monoclonal antibody Erbitux has shown quite good clinical activity in CRC, for which it has been approved for use.

When close examination of NSCLC responders was made, it was found that the majority of good responders had one of a few single mutations in EGFR (sm-EGFR), with those containing wild-type receptor (wt-EGFR) usually not responding appreciably, regardless of expression level. Such mutations are very rare in CRC, which tends towards overexpressed wt-EGFR, or overexpressed autocrine ligand expression. When these mutants, especially EGFR L858R, and EGFR del746-750, were analyzed it was found that they have the properties of being both intrinsically activated, which means that they were driving proliferation without an external signal, and also binding ATP more weakly than wt EGFR, (higher Kₘ) whilst having similar affinity to wt EGFR for the inhibitors. This meant that, as these inhibitors are ATP-competitive, that it was easier to compete ATP off the enzyme and shut down kinase activity in susceptible mutants than in the wt, giving a de facto boost to inhibitor potency in the mutants. At the same time these tumors had become more dependent on EGFR signaling for proliferation and survival than most tumors, because the signals had been reliably overactive ever since the original mutation event.

As stated earlier, solid tumors such as lung cancers are usually quite old by the time they are discovered, probably on average being 6-12 years beyond the arising of the original transformed founder cell. One of the properties of transformed cells is that they lose control over their DNA replication quality control, so their spontaneous mutation rate is much higher than that of untransformed cells. As mutations occur most easily during DNA replication, and these cells are replicating very quickly, this adds further to the mutation rate. The result is that as a tumor ages it will pick up an ever-increasing number of mutations, and it does so in a stochastic fashion, so that sub-clones of the tumor arise over time with somewhat different genetics from the original tumor, and one another. These sub-clones are not only involved in a survival struggle with the body itself, but with one another as they compete amongst themselves for the limited resources available to them. If one changes the environment for the dominant tumor clone, such that it becomes relatively less well adapted to its new environment, for example by adding an effective inhibitor to it, a previously much less successful minor clone may be able to take over the niche being vacated, if it is not as affected by said inhibitor. Alternatively, unless one either kills the clone outright, or completely shuts down proliferation, it will continue to spawn mutations, and if a mutation gets around the inhibition, this sub-clone will now be free to proliferate, without hindrance from either the inhibitor or the inhibited parental clone. Thus natural selection predicts that cancers, just like infectious diseases, should be able to develop drug resistance, and as the selection process is largely driven by competition between tumor sub-clones within a single host, the overall effect is to favor more aggressive sub-clones, and tumors generally become more deadly as they evolve.

When responders to gefitinib and erlotinib were followed, it was found that the onset of resistance could be correlated with several different genetic changes. In rare cases the tumors seem to pick up a totally different signaling system to drive the tumor, but usually the resistance involves tweaking of the original system. EGFR is a member of the erbB (Type I) subfamily of RTKs, along with erbB-2, erbB-3 and erbB-4. These receptors are activated by ligands which induce them to dimerize, and although EGFR-EGFR homodimers are quite commonly used in signaling, the more usual course in this family is for the ligands to induce heterodimerization, such that the signaling entity will be for example EGFR:erbB-2 or erb-B2:erbB-3 and an appropriate ligand. The simplest way to reactivate the system is to increase the expression of one of the other erbBs, and this is frequently seen, even before treatment, and may help to explain why a lot of wt EGFR overexpressing tumors do not respond to EGFR inhibition. A somewhat related mechanism involves the RTK HGFR, which although not a erbB family member has been shown to form oncogenic heterodimers with erbB family members, especially erbB-3, when overexpressed, and overexpression of HGFR is a common resistance mechanism to EGFR inhibitors. At least in laboratory settings, addition of an HGFR inhibitor to these cells restores sensitivity to EGFR inhibitors. The third, and commonest, mode of resistance is a further mutation in EGFR, giving doubly mutant receptor (dm-EGFR) which reduces its sensitivity to the EGFR inhibitor. The commonest of these is the so-called “gatekeeper” mutation T790M, and NSCLCs with double mutants such as L858R/T790M are commonly seen in initial responders, who have subsequently developed resistance to EGFR inhibitors. Whether such sub-clones were present all along, or whether they only arise after treatment is not known, but it seems most probable that the mutation is already present in short term responders, and may arise as a de novo mutation in long term responders who develop resistance late.

Initially, it was believed that these mutations block the inhibitors sterically from binding to the mutant enzyme, hence reducing their affinity, and efficacy. However, more recent studies suggest that the commonest mutations have very little effect on inhibitor affinity, but lead to restoration of ATP-binding affinity to that of wt EGFR, or possibly up to 10-fold greater, with the result that the achievable concentrations of the inhibitors are no longer high enough to shut down signaling to a therapeutically useful extent. In principle, one simply needs to improve the affinity of the inhibitors enough to overcome the increased ATP affinity, but in practice this is very difficult to do, because gefitinib and erlotinib are already very potent, subnanomolar, EGFR inhibitors with good PK properties, and yet have mediocre activity against tumors driven by wt EGFR. Furthermore, although the T790M mutant does not reduce the affinity of EGFR for erlotinib and gefitinib, it does limit the ways that one could increase affinity in the anilinoquinazoline chemotype of these two inhibitors. Therefore, to find greater affinity for the T790M-type mutants, new chemical templates have been examined, and some, especially U-shaped inhibitors of the type discussed later, appear to have considerable promise in this area.

EGFR receptors play an important role throughout the body, especially in the entire gastrointestinal epithelium and skin, which are both proliferatively very active tissues. As two of the major, dose-limiting toxicities of EGFR inhibitors are skin rashes and serious GI disturbances, these are almost certainly largely mechanism-based toxicities. As long as the tumor is driven by wt EGFR this is very difficult to avoid by rational design, especially for an oral agent, where GI tract exposure is obligate, but if the tumor is driven by mutant EGFR, one may be able to mitigate the toxicity seen with the approved drugs. For NSCLCs which respond to EGFR inhibitors, the initial target is not wt-EGFR, but one of a limited number of sm-EGFRs, and the later target is a dm-EGFR, both of which should at least in principle have different Structure-Activity Relationships (SARs) to wt-EGFR, giving one at least the theoretical possibility of reducing side effects by finding inhibitors which have considerably better affinity for sm- and/or dm-EGFR over wt-EGFR. Due to the similarity between EGFR and the mutant-EGFRs, and the fact that the original inhibitors only worked because they already were better inhibitors of sm-EGFR than wt-EGFR, not due to intrinsic affinity, but ATP-competition, this might be expected to be a difficult feat to accomplish. Unfortunately, clinical observation suggests that the aberrant EGFR systems driving tumors need to be very heavily suppressed to produce meaningful efficacy, whereas the suppression of wt-EGFR signaling in normal tissues at high enough levels to induce limiting toxicities is relatively easy to accomplish. However EGFR inhibitors with enhanced affinity for EGFR mutants, especially T790M dm-EGFRs have been found and examples of many of these are in the literature, with several now in clinical trials. This patent application describes compounds which fit one of these criteria.

Inhibitors of EGFR which have considerably greater affinity for a mutant EGFR than the wt EGFR should at an optimal dose be able to inhibit proliferation in tumors driven by that mutant, whilst having relatively little, if any effect on EGFR signaling in untransformed tissues, where wt EGFR is responsible for the EGFR signaling. This should allow considerably larger doses of mutant-selective EGFR inhibitors to be given, increasing both the efficacy against the mutant-driven tumor and the therapeutic index. It should be noted that because of mutant effects on ATP-binding, that is essentially what is already happening with responders to erlotinib and gefitinib, where the responding mutants are actually more sensitive to the inhibitors than wt EGFR, due mainly to their diminished affinity for the competing ligand ATP. Several third generation EGFR inhibitors have now been revealed, with some in the clinic. These compounds are generally irreversible inhibitors, initially based off of a U-shaped dianilinopyrimidine scaffold, but this been extended to several related scaffolds, but all bind in a similar mode to the dianilinopyrimidines. In general these compounds are very potent inhibitors of the mutant EGFRs, containing the T790M mutation, and are somewhat less potent against wt EGFR, and some of the other mutations. Because of this profile, it is believed that the mechanism-based toxicities of wt EGFR inhibition should be considerably reduced, while retaining very strong inhibitory potency against tumors driven by the appropriate EGFR mutations. Thus compounds of this type may be especially useful as second line therapy, after a patient previously sensitive to first line erlotinib or gefitinib therapy becomes resistant. Not only will these inhibitors allow the appropriate mutant receptors to be inhibited as strongly as previously, but they should do this whilst themselves not inducing appreciable mechanism-induced toxicity through EGFR inhibition. The inhibitors of the present invention are irreversible inhibitors of EFGR, with a similar selective profile for mutant over wt EGFR inhibition to these agents, and excellent pharmacokinetic properties, and will therefore prove to be excellent agents for second line treatment of NSCLC, and any other tumors driven by this sub-family of mutated EGFR kinases.

Another method of increasing the potency of especially EGFR inhibitors was developed in the mid-1990s. Many sites on proteins are quite strongly nucleophilic, either because they are intrinsically nucleophilic, with cysteine thiols being the principle example, with lysine amines, histidine imidazoles, and serine, threonine and tyrosine hydroxyls also being less potent possibilities, or because they have been deliberately activated, as in the catalytic hydroxyls in many amidases. Such residues can often be targeted by electrophiles, which modify the protein under rather mild conditions. Depending on the function of the modified residue, and its position on the protein, this may or may not lead to a loss of enzyme function. It was realized that a subset of TKs use a cysteine residue on the edge of the ATP binding cleft to form a hydrogen bond to the ribose of ATP, whereas the majority use a threonine for this purpose. The EGFR family all contains this cysteine (C⁷⁹⁷ in EGFR). It was hypothesized that this cysteine could be alkylated by an alkylating moiety attached to an inhibitor, which bound in the ATP-binding site, and presented the electrophile in the vicinity of the cysteine sulfur. Indeed many of the first generation of EGFR inhibitors were potent electrophiles, which may well have targeted Cys⁷⁹⁷ or other nucleophiles on EGFR. Unfortunately, this inhibition did not lead to very potent inhibitors, nor did it lead to very selective inhibitors, suggesting that the electrophiles were reactive enough, and non-discriminating enough to react with a wide variety of proteins, especially kinases, and that in many of these cases the alkylation was occurring in either the catalytic domain, or a controlling “switch region” of the enzyme. To make this concept useful, the alkylating moiety would have to be of low intrinsic reactivity, because one does not want it to indiscriminately react with the vast array of nucleophiles in the body, both for potential PK and toxicity reasons. To get an alkylating agent to react with this necessarily rather weak electrophile with high selectivity, it was shown that the compound itself had to have both high (non-covalent) affinity for the binding site, and would have to bind preferentially in a conformation which placed the weak electrophile in close proximity to the electrophile. Lastly, it was also found that the reaction needed to be fast relative to the plasma half-life of the inhibitor, or most of it would wash out of the body without ever reacting with the crucial cysteine. Such irreversibly inhibitory compounds were discovered, and it was found that they not only were much more potent inhibitors of EGFR in vivo than the theoretically equipotent reversible inhibitors, but as a bonus they made (at least in the case of the anilinoquinazolines and the related 3-cyanoquinolines) a rather poor erbB-2 and erbB-4 inhibitory template into very potent inhibitors of all of the erbBs, demonstrating that if the binding mode were really good in its placement of the alkylating moiety, very high non-covalent affinity for the target might be less vital. Most of the second generation EGFR inhibitors which went into the clinic are irreversible inhibitors of EGFR, using acrylamide derivatives as electrophiles, and they appear to be more active in general in the clinic than reversible inhibitors, but they also tend to have higher toxicity, so only one, afatinib, has shown a good enough profile to gain approval.

Many different classes of kinase inhibitors have been developed, and several have been successfully approved and marketed. One of the molecular scaffolds which appears to produce potent inhibition of a large number of kinases, is a series of three concatenated rings, of which two, and frequently all three, are aromatic, which can form a U-shaped structure when binding to a kinase. The two distal rings can be directly linked to the central ring by bonds, or via various linkers consisting of 1-3 atom chains. The central ring, which is almost invariably a nitrogen-containing heteroaromatic system with an NH group adjacent to a ring nitrogen, forms 1-3 hydrogen bonds to the backbone of residues in the hinge domain of kinases, between the N- and C-terminal lobes, just prior to the so called DFG loop, an invariant structure in kinases, which has to be placed correctly for an active conformation of the enzyme to be achieved. This end of the inhibitor also occupies a part of the adenine-binding region of the kinase, which tends to be very hydrophobic, whereas the two rings, which make the “stems” of the U, occupy a broad channel frequently filling part of the space normally occupied by the rest of the ATP molecule. Although quite a lot of affinity for specific kinases comes from decorating these core rings with selected substituents which produce favorable interactions with hopefully unique structural determinants in the target kinases, and/or unfavorable interactions with kinases, which one does not wish to inhibit, a lot of the affinity and selectivity for various kinases comes from the various torsions and bend angles between the three rings, and some substituents which optimize affinity for the target kinase may not themselves interacting directly with the protein, but may control the most stable conformations of the three rings with respect to one another. Thus the purpose of some substituents can be to affect the overall internal energy of the inhibitory molecule, in order to stabilize a favorable conformation for binding, rather than directly interact with the kinase.

None of the first and second generation EGFR/erbB-2 inhibitors which entered the clinic show the U-shaped binding mode. They have the 4-anilino (or extended 4-anilino) group binding into a cleft between the 134 sheet and the αC-helix, which is behind the vital L⁷⁴⁵-D⁸⁵⁵ salt bridge, and the DFG loop of which D⁸⁵⁵ is a part.

### SUMMARY OF THE INVENTION

The present invention provides, in part, novel compounds and pharmaceutically acceptable salts, solvates, esters, and/or prodrugs thereof that can selectively modulate the activity of protein kinases especially of the Type I receptor tyrosine kinase (RTK) family, or erbB family, and most particularly of certain mutated forms of the EGFR receptor, which provide resistance to current EGFR-based inhibitory therapies. This inhibitory activity affects biological functions, including but not limited to, cell proliferation and cell invasiveness, inhibiting metastasis, inducing apoptosis or inhibiting angiogenesis. Also provided are pharmaceutical compositions and medicaments, comprising the compounds or salts of the invention, alone or in combination with other therapeutic agents or palliative agents.

In one embodiment, the present invention relates to a compound of the formula (I) or a stereoisomer or a pharmaceutically acceptable salt, solvate, ester, or prodrug thereof, as disclosed herein.

In one embodiment, the present disclosure relates to compounds of formula (A) or (B):

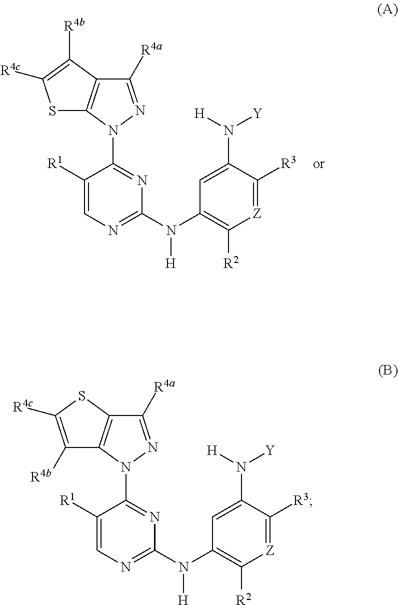

or a stereoisomer or a pharmaceutically acceptable salt, solvate, ester, or prodrug thereof;
wherein,
Z is CH or N;
Y is

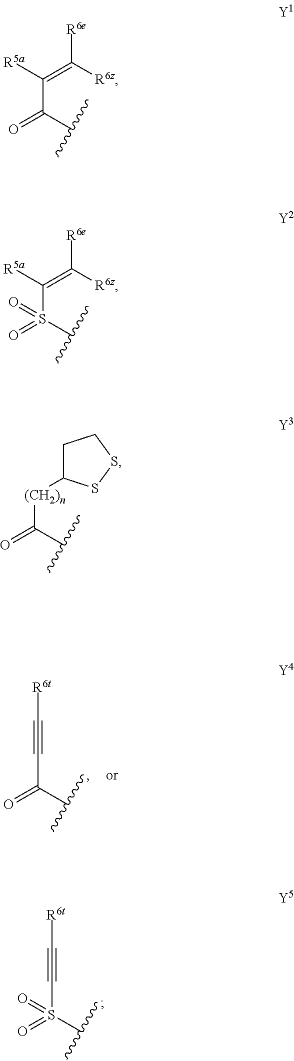

in Y¹ and Y², R^{5a} is H, F, Cl, CF₃, CHF₂, CF₂C₁₋₆ alkyl, CF₂CH₂NR⁸R⁹, CH₂NR⁸R⁹, CN, or C₁₋₆ alkyl;
in Y¹ and Y², R^{6e} is R¹⁰, H, F, aryl, heteroaryl, cycloalkyl, heterocycloalkyl, (CH₂)ₘCHR¹⁰R⁷, CF₂(CH₂)ₘCHR¹⁰R⁷, or C(R¹⁰)₂R;
in Y⁴ and Y⁵, R^{6t} is C₁₋₆ alkyl, C₃₋₆ cycloalkyl, aryl, heteroaryl, heterocycloalkyl, (CH₂)ₘCHR¹⁰R⁷, C(R¹⁰)₂R;
in Y¹ and Y², R^{6Z} is H, F, Cl, CF₃, CHF₂, CF₂C₁₋₆ alkyl or C₁₋₆ alkyl; or
alternatively in Y¹ and Y², R^{6e} and R^{6z}, taken together, form ═CR^{6e′}R^{6z′} (allene), wherein R^{6e′} is R¹⁰, H, F, aryl, heteroaryl, cycloalkyl, heterocycloalkyl, (CH₂)ₘCHR¹⁰R⁷, CF₂(CH₂)ₘCHR¹⁰R⁷, or C(R¹⁰)₂R⁷ and wherein, R^{6z′} is H, F, Cl, CF₃, CHF₂, CF₂C₁₋₆ alkyl or C₁₋₆ alkyl; or
alternatively in Y¹ and Y², R^{6e} and R^{6z}, taken together with the sp² carbon atom to which both are attached, form an alicyclic ring of 4 to 7 members wherein one of the ring atoms are optionally replaced by NR⁸, O, S(O)ₓ, S(═O)(═NR⁸), P═O, P(═O)(OR⁸), OP(═O)(OR⁸)O, and the alicyclic ring is optionally substituted with one or more substituents selected from the group consisting of halogen, oxo, OH, OR⁸, and NR⁸R⁹;
R¹ is independently selected from hydrogen, fluoro, chloro, bromo, methyl, ethyl, hydroxyl, methoxy, ethoxy, isopropoxy, cyclopropoxy, —OCF₃, —OCH₂CF₃, —OCH₂CHF₂, ethenyl, ethynyl, —CF₃, —CHF₂, —CHO, —CH₂OH, —CONH₂, —CO₂Me, —CONHMe, —CONMe₂, and cyano;
R² is —OCF₃, —OCHF₂, —OCF₂CF₃, —OCH₂CHF₂, —OCH₂CF₃, cyclopropyl, cyclopropoxy, methoxy, —OCD₃, ethoxy, or isopropoxy;
R³ is —N(R¹⁰)C₂₋₆ alkyl-NR¹⁰R¹⁰, —N(R¹⁰)C₂₋₆ alkyl-R⁷, —O(CH₂)ₚR⁷, —N(R¹⁰)C(═O)(CH₂)ₚR⁷, or R⁷;
each R^{4a}, R^{4b}, and R^{4c} are independently H, cyano, nitro, halo, —C₁₋₆ alkyl, —C₁₋₆ haloalkyl, -carboxy-C₁₋₆ alkyl, —C₁₋₆ hydroxyalkyl, R⁸R⁹N—C₁₋₆ alkyl-, —C₂₋₆ alkenyl, —C₂₋₆ alkynyl, C₁₋₆ acyl-, R⁷—(CH₂)ₚC(═O)—, C₁₋₆ hydroxyalkyl-C(═O)—, carboxy, —C₁₋₆ alkoxycarbonyl, —C(═O)NR⁸R⁹, hydroxyl, —C₁₋₆ alkoxy, —C₁₋₆ acyloxy, —NR⁸R⁹, C₁₋₆ acyl-N(R¹⁰)—, pyrazole, 1,2,3-triazole, tetrazole, (C₁₋₆ alkyl)SO₂—, or R⁷SO₂—;
R⁷ is OH, NR⁸R⁹, O(CH₂)_{q}NR⁸R⁹, C₁₋₆ alkoxy, C₁₋₆ alkoxy-C₁₋₆ alkoxy, C₂₋₆ hydroxyalkoxy, oxetanyl, oxetanyloxy, oxetanylamino, oxolanyl, oxolanyloxy, oxolanylamino, oxanyl oxanyloxy, oxanylamino, oxepanyl, oxepanyloxy, oxepanylamino, azetidinyl, azetidinyloxy, azetidylamino, pyrrolidinyl, pyrolidinyloxy, pyrrolidinylamino, piperidinyl, piperidinyloxy, piperidinylamino, azepanyl, azepanyloxy, azepanylamino, dioxolanyl, dioxanyl, morpholino, thiomorpholino, thiomorpholino-S,S-dioxide, piperazino, dioxepanyl, dioxepanyloxy, dioxepanylamino, oxazepanyl, oxazepanyloxy, oxazepanylamino, diazepanyl, diazepanyloxy, diazepanylamino, (3R)-3-(dimethylamino)pyrrolidin-1-yl, (3S)-3-(dimethylamino)pyrrolidin-1-yl, 3-(dimethylamino)azetidin-1-yl, [2-(dimethylamino)ethyl](methyl)amino, [2-(methylamino)ethyl](methyl)amino, 5-methyl-2,5diazaspiro[3.4]oct-2-yl, (3aR,6aR)-5-methylhexa-hydro-pyrrolo[3,4-b]pyrrol-1(2H)-yl, I-methyl-1,2,3,6-tetrahydropyridin-4-yl, 4-methylpiperizin-1-yl, 4-[2(dimethylamino)-2-oxoethyl]piperazin-1-yl, methyl[2-(4-methylpiperazin-1yl)ethyl]amino, methyl[2-(morpholin-4-yl)ethyl]amino, 1-amino-1,2,3,6tetrahydropyridin-4-yl, 4-[(2S)-2-aminopropanoyl]piperazin-1-yl, all of which may be optionally substituted with OH, OR¹⁰, oxo, halogen, R¹⁰, CH₂OR¹⁰, or CH₂NR⁸R⁹;

R⁸ and R⁹ are each independently H, —CD₃, C₁₋₆ alkyl, C₃₋₆ alkenyl, C₃₋₆ alkynyl, C₃₋₈ cycloalkyl, —(C₁₋₃ alkyl)-(C₃₋₈ cycloalkyl), C₃₋₈ cycloalkenyl, C₁-C₆ acyl, 4-12 membered monocyclic or bicyclic heterocyclyl, 4-12 membered monocyclic or bicyclic heterocyclyl-C₁—C₆ alkyl-, C₆-C₁₂ aryl, 5-12 membered heteroaryl; wherein R⁸ and R⁹ may be further independently substituted with up to three substituents chosen from hydroxyl, C₁₋₆ alkoxy, C₁₋₆ hydroxyalkyl, C₁₋₆ alkoxy-C₁₋₆ alkyl, C₁₋₆ alkoxy-C₁₋₆ alkoxy, C₂₋₆ hydroxyalkoxy, oxo, thiono, cyano or halo; or
alternatively, R⁸ and R⁹, taken together with the N atom to which they are both attached, form a heterocyclic ring of 4-7 members, containing up to one other heteroatom selected from O, S, or NR¹¹, or a heterobicyclic ring of 7-12 members which may be fused, bridged or spiro, and contain up to two other heteroatoms chosen from O, S(O)ₓ, or NR¹¹, and these heterocyclic rings are optionally substituted with up to three substituents chosen from hydroxyl, C₁₋₆ alkoxy, C₁₋₆ hydroxyalkyl, C₁₋₆ alkoxy-C₁₋₆ alkyl, C₁₋₆ alkoxy-C₁₋₆ alkoxy, C₂₋₆ hydroxyalkoxy, oxo, thiono, cyano or halo;
each R¹⁰ is independently H, —CD₃, C₁₋₆ alkyl, C₃₋₆ cycloalkyl, C₂₋₆ hydroxyalkyl, C₁₋₆ alkoxy-C₁₋₆ alkyl or C₂₋₆ alkyl-NR⁸R⁹;
alternatively, two R¹⁰ on the same N atom to which they are both attached, form a heterocyclic ring of 5-6 members, containing up to one other heteroatom selected from O, S, or NR¹¹;
each R¹¹ is independently hydrogen or C₁-C₆ alkyl, which is optionally substituted with up to three substituents selected from hydroxyl, oxo, thiono, cyano or halo;
m is 0, 1, 2, or 3;
n is 1, 2, or 3;
q is 2, 3, or 4;
p is 0, 1, 2, 3, or 4; and
x is 0, 1, or 2.

In one embodiment, the present disclosure relates to compounds having the structure of formula (A):

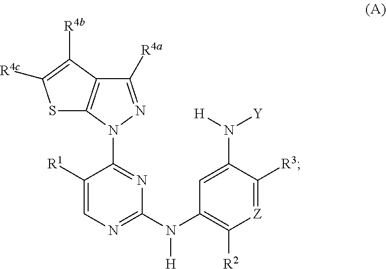

or a stereoisomer or a pharmaceutically acceptable salt, solvate, ester, or prodrug thereof;
wherein,
Z is CH or N;
R¹ is selected from hydrogen, fluoro, chloro, bromo, methyl, CF₃, CHF₂, and cyano;
R² is —OCF₃, —OCHF₂, —OCF₂CF₃, —OCH₂CHF₂, —OCH₂CF₃, cyclopropyl, cyclopropoxy, methoxy, —OCD₃, ethoxy, or isopropoxy;
R³ is N(R¹⁰)C₂₋₆ alkyl-NR¹⁰R¹⁰;
R^{4a}, R^{4b} and R^{4c} are each independently H, cyano, halo, —C₁₋₆ alkyl, —C₁₋₆ haloalkyl, carboxy-C₁₋₆ alkyl, —C₁₋₆ hydroxyalkyl, R⁸R⁹N—C₁₋₆ alkyl-, —C₂₋₆ alkenyl, —C₂₋₆ alkynyl, C₁₋₆ acyl-, R—(CH₂)ₚC(═O)—, C₁₋₆ hydroxyalkyl-C(═O)—, carboxy, —C₁₋₆ alkoxycarbonyl, —C(═O)NR⁸R⁹, hydroxyl, alkoxy, C₁₋₆ acyloxy, —NR⁸R⁹, C₁₋₆ acyl-N(R¹⁰)—, R⁷SO₂—,
R⁷ is OH, NR⁸R⁹, O(CH₂)_{q}NR⁸R⁹, C₁₋₆ alkoxy, or C₂₋₆ hydroxyalkoxy;
R⁸ and R⁹ are independently H, —CD₃, C₁₋₆ alkyl, C₃₋₆ alkenyl, C₃₋₆ alkynyl, C₃₋₈ cycloalkyl, C₃₋₈ cycloalkenyl, C₁-C₆ acyl, 4-12 membered monocyclic or bicyclic heterocyclyl, 4-12 membered monocyclic or bicyclic heterocyclyl-C₁-C₆ alkyl-, C₆-C₁₂ aryl, 5-12 membered heteroaryl; and R⁸ and R⁹ may be further independently substituted with up to three substituents chosen from hydroxyl, C₁₋₆ alkoxy, C₁₋₆ hydroxyalkylC₂₋₆ hydroxyalkoxy, oxo, thiono, cyano or halo; or
alternatively, R⁸ and R⁹, taken together with the N atom to which they are both attached, form a heterocyclic ring of 4-7 members, containing up to one other heteroatom selected from O, S, or NR¹¹, or a heterobicyclic ring of 7-12 members which may be fused, bridged or spiro, and contain up to two other heteroatoms chosen from O, S(O)ₓ, or NR¹¹, and these heterocyclic rings are optionally substituted with up to three substituents chosen from hydroxyl, C₁₋₆ alkoxy, C₁₋₆ hydroxyalkyl, C₁₋₆ alkoxy-C₁₋₆ alkyl, C₁₋₆ alkoxy-C₁₋₆ alkoxy, C₂₋₆ hydroxyalkoxy, oxo, thiono, cyano or halo;
each R¹⁰ is independently H, —CD₃, C₁₋₆ alkyl, C₃₋₆ cycloalkyl, C₂₋₆ hydroxyalkyl, C₁₋₆ alkoxy-C₁₋₆ alkyl or C₂₋₆ alkyl-NR⁸R⁹; or
p is 0, 1, 2, 3, or 4;
q is 2, 3, or 4; and
x is 0, 1, or 2.

In one embodiment, R³ of formula (A) or (B) is —N(CH₃)CH₂CH₂NR¹⁰R¹⁰.

In one embodiment, R¹⁰ of formula (A) or (B) is each independently H, —CD₃, C₁₋₆ alkyl, C₃₋₆ cycloalkyl, or C₂₋₆ hydroxyalkyl. In other embodiments, R¹⁰ is each independently H, —CD₃, methyl, ethyl, or isopropyl.

In one embodiment, Y of formula (A) or (B) is

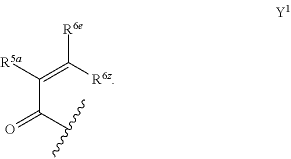

In one embodiment, R^{5a}, R^{6e}, and R^{6z} are each H.

In one embodiment, R^{4a} of formula (A) or (B) is H, —C₁₋₆ alkyl, or —NR⁸R⁹.

In one embodiment, R⁸ and R⁹ of formula (A) or (B) are independently H, —CD₃, or C₁₋₆ alkyl.

In one embodiment, R^{4b} and R^{4c} of formula (A) or (B) are each independently H, cyano, F, Cl, Br, —C₁₋₆ alkyl, CF₃, CHF₂, CONH₂ or C(═O)NR⁸R⁹. In one embodiment, R^{4b} and R^{4c} of formula (A) or (B) are each independently H, cyano, F, Cl, Br, CH₃, CF₃, CHF₂, CONH₂ or C(═O)NR⁸R⁹.

In one embodiment, the present disclosure relates to compounds having the structure of formula (C):

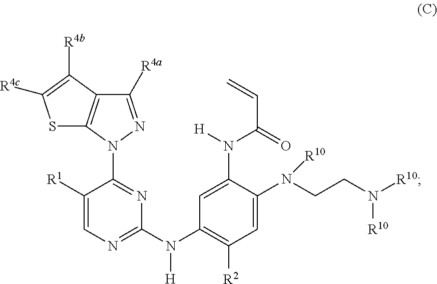

or a stereoisomer or a pharmaceutically acceptable salt, solvate, ester, or prodrug thereof;
wherein,
R¹ is hydrogen, fluoro, chloro, or methyl;
R² is —OCF₃, —OCHF₂, —OCF₂CF₃, —OCH₂CHF₂, —OCH₂CF₃, cyclopropyl, cyclopropoxy, methoxy, —OCD₃, ethoxy, or isopropoxy;
R^{4a} is H or —NR⁸R⁹;
R^{4b} and R^{4c} are each independently H, cyano, F, Cl, Br, CH₃, CF₃, CHF₂, CONH₂, or C(═O)NR⁸R⁹;
R⁸ and R⁹ are each independently H, —CD₃, or C₁₋₆ alkyl; and
each R¹⁰ is independently H, —CD₃, C₁₋₆ alkyl, C₃₋₆ cycloalkyl, or C₂₋₆ hydroxyalkyl.

In one embodiment, the compound of formula (C) comprises:
R¹ is hydrogen;
R² is methoxy, —OCD₃, ethoxy, or isopropoxy;
R^{4a} is NR⁸R⁹;
R^{4b} is H, or CH₃;
R^{4c} is H, F, Cl, Br, or CH₃;
R⁸ and R⁹ are each independently H, —CD₃, —CH₃, —CH₂CH₃, or —CH(CH₃)₂; and
each R¹⁰ is independently H, —CD₃, —CH₃, —CH₂CH₃, or —CH(CH₃)₂.

In one embodiment, the compound of the present disclosure has the structure of (C-I):

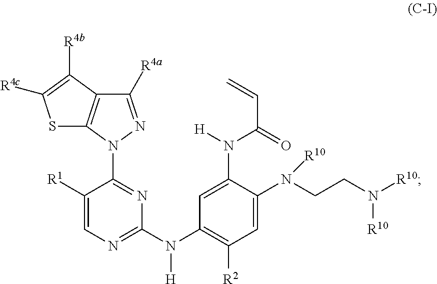

or a stereoisomer or a pharmaceutically acceptable salt, solvate, ester, or prodrug thereof;
wherein, R¹ is hydrogen, fluoro, chloro, or methyl;
R² is —OCF₃, —OCHF₂, —OCF₂CF₃, —OCH₂CHF₂, —OCH₂CF₃, cyclopropyl, cyclopropoxy, methoxy, —OCD₃, ethoxy, or isopropoxy; R^{4a} is H or —NR⁸R⁹;
R^{4b} and R^{4c} are each independently H, cyano, F, Cl, Br, —C₁₋₆ alkyl, —CF₃, —CHF₂, —CONH₂, or —C(═O)NR⁸R⁹;
R⁸ and R⁹ are each independently H, —CD₃, or —C₁₋₆ alkyl; and
each R¹⁰ is independently H, —CD₃, —C₁₋₆ alkyl, —C₃₋₆ cycloalkyl, or —C₂₋₆ hydroxyalkyl.

In another embodiment, the compound of formula (C-I) comprises:
R¹ is hydrogen;
R² is methoxy, —OCD₃, ethoxy, or isopropoxy;
R^{4a} is NR⁸R⁹;
R^{4b} is H, or CH₃;
R^{4c} is H, F, Cl, Br, —CF₃, —CH₃, or —CH₂CH₃;
R⁸ and R⁹ are each independently H, —CD₃, —CH₃, —CH₂CH₃, or —CH(CH₃)₂; and
each R¹⁰ is independently H, —CD₃, —CH₃, —CH₂CH₃, or —CH(CH₃)₂.

In one embodiment, a compound of formula (A), (B), or (C) is:

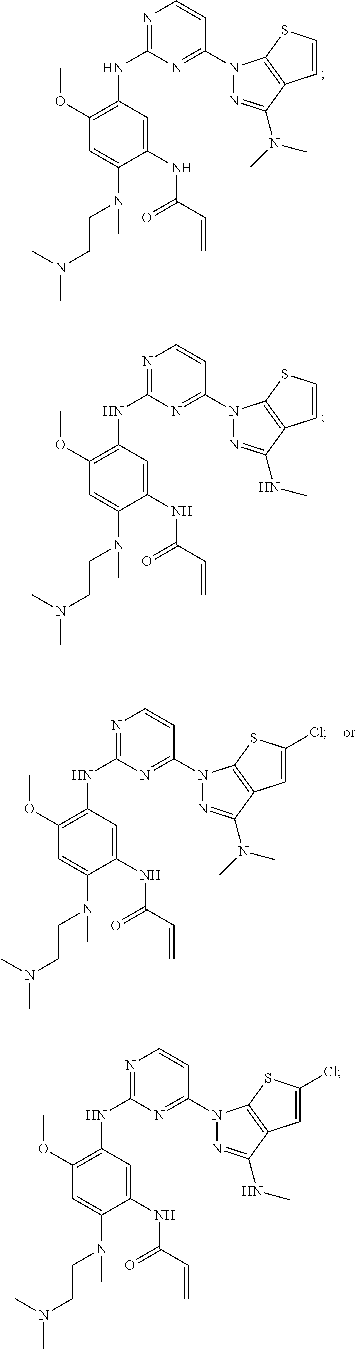 [SMILES: C=CC(=O)Nc1cc(Nc2nccc(-n3nc(N(C)C)c4ccsc43)n2)c(OC)cc1N(C)CCN(C)C ; C=CC(=O)Nc1cc(Nc2nccc(-n3nc(NC)c4ccsc43)n2)c(OC)cc1N(C)CCN(C)C ; C=CC(=O)Nc1cc(Nc2nccc(-n3nc(N(C)C)c4cc(Cl)sc43)n2)c(OC)cc1N(C)CCN(C)C ; C=CC(=O)Nc1cc(Nc2nccc(-n3nc(NC)c4cc(Cl)sc43)n2)c(OC)cc1N(C)CCN(C)C]

or a stereoisomer or a pharmaceutically acceptable salt, solvate, ester, or prodrug thereof.
In one embodiment, a compound of formula (A), (B), (C) or (C-I) is:

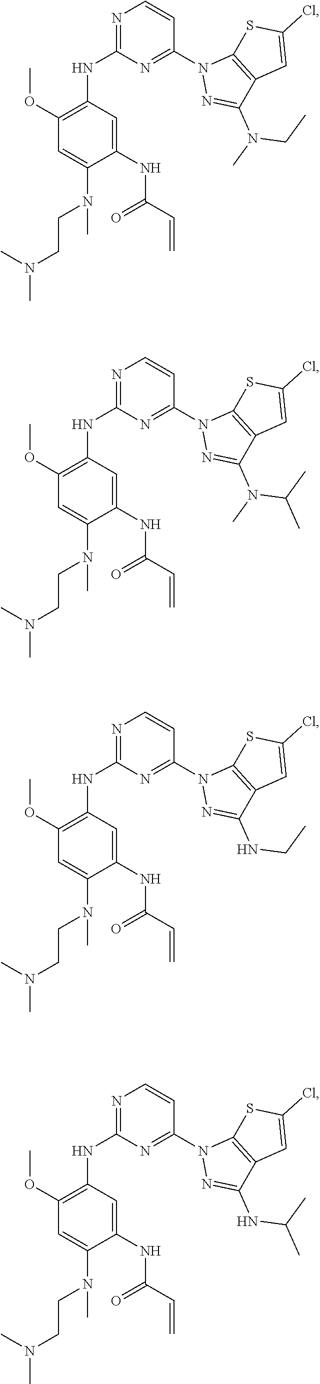 [SMILES: C=CC(=O)Nc1cc(Nc2nccc(-n3nc(N(C)CC)c4cc(Cl)sc43)n2)c(OC)cc1N(C)CCN(C)C ; C=CC(=O)Nc1cc(Nc2nccc(-n3nc(N(C)C(C)C)c4cc(Cl)sc43)n2)c(OC)cc1N(C)CCN(C)C ; C=CC(=O)Nc1cc(Nc2nccc(-n3nc(NCC)c4cc(Cl)sc43)n2)c(OC)cc1N(C)CCN(C)C ; C=CC(=O)Nc1cc(Nc2nccc(-n3nc(NC(C)C)c4cc(Cl)sc43)n2)c(OC)cc1N(C)CCN(C)C]

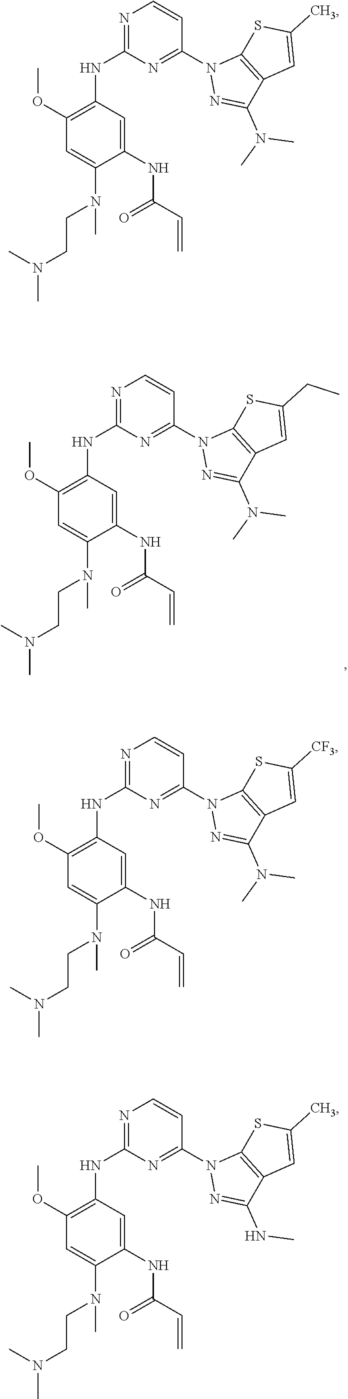

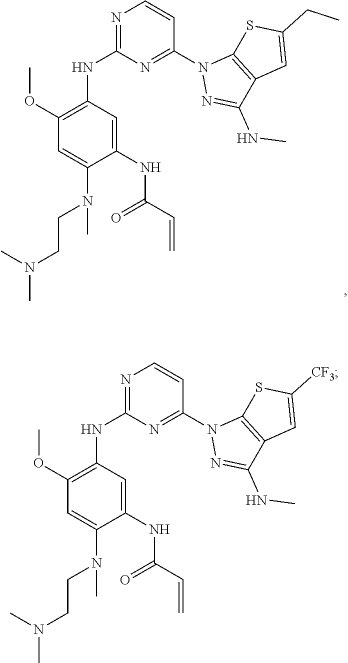

or a stereoisomer or a pharmaceutically acceptable salt, solvate, ester, or prodrug thereof.

In one embodiment, the present disclosure relates to compounds of formula (D):

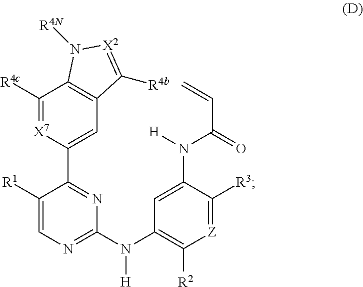

or a stereoisomer or a pharmaceutically acceptable salt, solvate, ester, or prodrug thereof;
wherein,
Z is CH or N;
X² and X⁷ are each CH, CR⁴, or N;
R¹ is hydrogen, fluoro, chloro, bromo, methyl, ethyl, hydroxyl, methoxy, ethoxy, isopropoxy, cyclopropoxy, —OCF₃, —OCH₂CF₃, —OCH₂CHF₂, ethenyl, ethynyl, CF₃, CHF₂, CHO, CH₂OH, CONH₂, CO₂Me, CONHMe, CONMe₂, or cyano;
R² is —OCF₃, —OCHF₂, —OCF₂CF₃, —OCH₂CHF₂, —OCH₂CF₃, cyclopropyl, cyclopropoxy, methoxy, —OCD₃, ethoxy, or isopropoxy;
R³ is N(R¹⁰)C₂₋₆ alkyl-NR¹⁰R¹⁰;
each R⁴ is independently H, cyano, halo, —C₁₋₆ alkyl, —C₁₋₆ haloalkyl, carboxy-C₁₋₆ alkyl, —C₁₋₆ hydroxyalkyl, R⁸R⁹N—C₁₋₆ alkyl-, —C₂₋₆ alkenyl, —C₂₋₆ alkynyl, C₁₋₆ acyl-, R⁷—(CH₂)ₚC(═O)—, C₁₋₆hydroxyalkyl-C(═O)—, carboxy, —C₁₋₆ alkoxycarbonyl, —C(═O)NR⁸R⁹, hydroxyl, alkoxy, C₁₋₆ acyloxy, —NR⁸R⁹, C₁₋₆ acyl-N(R¹⁰)—, or R⁷SO₂—; and
R⁸ and R⁹ are independently H, —CD₃, C₁₋₆ alkyl, C₃₋₆ alkenyl, C₃₋₆ alkynyl, C₃₋₈ cycloalkyl, C₃₋₈ cycloalkenyl, C₁-C₆ acyl, 4-12 membered monocyclic or bicyclic heterocyclyl, 4-12 membered monocyclic or bicyclic heterocyclyl-C₁-C₆ alkyl-, C₆-C₁₂ aryl, 5-12 membered heteroaryl; and R⁸ and R⁹ may be further independently substituted with up to three substituents chosen from hydroxyl, C₁₋₆ alkoxy, C₁₋₆ hydroxyalkylC₂₋₆ hydroxyalkoxy, oxo, thiono, cyano or halo; or
alternatively, R⁸ and R⁹, taken together with the N atom to which they are both attached, form a heterocyclic ring of 4-7 members, containing up to one other heteroatom selected from O, S, or NR¹¹, or a heterobicyclic ring of 7-12 members which may be fused, bridged or spiro, and contain up to two other heteroatoms chosen from O, S(O)ₓ, or NR¹¹, and these heterocyclic rings are optionally substituted with up to three substituents chosen from hydroxyl, C₁₋₆ alkoxy, C₁₋₆ hydroxyalkyl, C₁₋₆ alkoxy-C₁₋₆ alkyl, C₁₋₆ alkoxy-C₁₋₆ alkoxy, C₂₋₆ hydroxyalkoxy, oxo, thiono, cyano or halo;
R^{4b} is H, halo, —C₁₋₆ alkyl, or —C₁₋₆ haloalkyl;
R^{4c} is cyano, C₁₋₆ acyl-, —C(═O)NR⁸R⁹, hydroxyl, alkoxy, or F;
R^{4N} is H, —CD₃, or —C₁₋₆ alkyl;
R⁷ is OH, NR⁸R⁹, —O(CH₂)_{q}NR⁸R⁹, C₁₋₆ alkoxy, or C₂₋₆ hydroxyalkoxy;
each R¹⁰ is independently H, —CD₃, C₁₋₆ alkyl, C₃₋₆ cycloalkyl, C₂₋₆ hydroxyalkyl, C₁₋₆ alkoxy-C₁₋₆ alkyl or C₂₋₆ alkyl-NR⁸R⁹;
p=0, 1, 2, 3, or 4;
q=2, 3, or 4; and
x=0, 1, or 2.

In one embodiment, the present disclosure relates to compounds of formula (D-I):

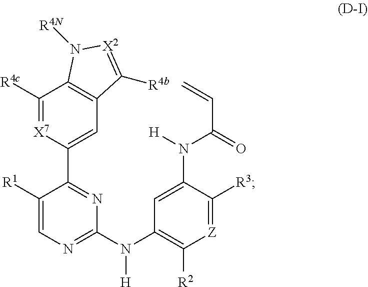

or a stereoisomer or a pharmaceutically acceptable salt, solvate, N-oxide, ester, or prodrug thereof;
wherein,
Z is CH or N;
X² and X⁷ are each CH, CR⁴, or N;
R¹ is hydrogen, fluoro, chloro, bromo, methyl, ethyl, hydroxyl, methoxy, ethoxy, isopropoxy, cyclopropoxy, —OCF₃, —OCH₂CF₃, —OCH₂CHF₂, ethenyl, ethynyl, CF₃, CHF₂, CHO, CH₂OH, CONH₂, CO₂Me, CONHMe, CONMe₂, or cyano;
R² is —OCF₃, —OCHF₂, —OCF₂CF₃, —OCH₂CHF₂, —OCH₂CF₃, cyclopropyl, cyclopropoxy, methoxy, —OCD₃, ethoxy, or isopropoxy;
R³ is —N(R¹⁰)(C₂₋₆ alkyl)-NR¹⁰R¹⁰ or —N(R¹⁰)(C₃₋₁₀ cycloalkylalkyl)-NR¹⁰R¹⁰;
each R⁴ is independently H, cyano, halo, —C₁₋₆ alkyl, —C₁₋₆ haloalkyl, carboxy-C₁₋₆ alkyl, —C₁₋₆ hydroxyalkyl, R⁸R⁹N—C₁₋₆ alkyl-, —C₂₋₆ alkenyl, —C₂₋₆ alkynyl, C₁₋₆ acyl-, R⁷—(CH₂)ₚC(═O)—, C₁₋₆ hydroxyalkyl-C(═O)—, carboxy, —C₁₋₆ alkoxycarbonyl, —C(═O)NR⁸R⁹, hydroxyl, alkoxy, C₁₋₆ acyloxy, —NR⁸R⁹, C₁₋₆ acyl-N(R¹⁰)—, or R⁷SO₂—; and
R⁸ and R⁹ are independently H, —CD₃, C₁₋₆ alkyl, C₃₋₆ alkenyl, C₃₋₆ alkynyl, C₃₋₈ cycloalkyl, C₃₋₈ cycloalkenyl, C₁-C₆ acyl, 4-12 membered monocyclic or bicyclic heterocyclyl, 4-12 membered monocyclic or bicyclic heterocyclyl-C₁-C₆ alkyl-, C₆-C₁₂ aryl, 5-12 membered heteroaryl; and R⁸ and R⁹ may be further independently substituted with up to three substituents chosen from hydroxyl, C₁₋₆ alkoxy, C₁₋₆ hydroxyalkylC₂₋₆ hydroxyalkoxy, oxo, thiono, cyano or halo; or
alternatively, R⁸ and R⁹, taken together with the N atom to which they are both attached, form a heterocyclic ring of 4-7 members, containing up to one other heteroatom selected from O, S, or NR¹¹, or a heterobicyclic ring of 7-12 members which may be fused, bridged or spiro, and contain up to two other heteroatoms chosen from O, S(O)ₓ, or NR¹¹, and these heterocyclic rings are optionally substituted with up to three substituents chosen from hydroxyl, C₁₋₆ alkoxy, C₁₋₆ hydroxyalkyl, C₁₋₆ alkoxy-C₁₋₆ alkyl, C₁₋₆ alkoxy-C₁₋₆ alkoxy, C₂₋₆ hydroxyalkoxy, oxo, thiono, cyano or halo;
R^{4b} is H, halo, —C₁₋₆ alkyl, or —C₁₋₆ haloalkyl;
R^{4c} is H, cyano, hydroxyl, alkoxy, —C₁₋₆ alkyl, or —C₁₋₆ haloalkyl, Cl, or F, provided that when R^{4c} is H, R^{4b} is halo, —C₁₋₆ alkyl, or —C₁₋₆ haloalkyl;
R^{4N} is H, —CD₃, or —C₁₋₆ alkyl;
R⁷ is OH, NR⁸R⁹, —O(CH₂)_{q}NR⁸R⁹, C₁₋₆ alkoxy, or C₂₋₆ hydroxyalkoxy;
each R¹⁰ is independently H, —CD₃, C₁₋₆ alkyl, C₃₋₆ cycloalkyl, C₂₋₆ hydroxyalkyl, C₁₋₆ alkoxy-C₁₋₆ alkyl or C₂₋₆ alkyl-NR⁸R⁹; or
alternatively, two R¹⁰ on the same N atom, taken together form a heterocyclic ring of 3-7 members, optionally substituted with up to three substituents chosen from hydroxyl, C₁₋₆ alkoxy, C₁₋₆ hydroxyalkyl, C₁₋₆ alkoxy-C₁₋₆ alkyl, C₁₋₆ alkoxy-C₁₋₆ alkoxy, C₂₋₆ hydroxyalkoxy, oxo, thiono, cyano or halo;
p=0, 1, 2, 3, or 4;
q=2, 3, or 4; and
x=0, 1, or 2.

In one embodiment of the compound of formula (D-I),
X² is CH or CR⁴;
R⁴ is methyl, ethyl, or isopropyl;
R^{4c} is cyano, —CF₃, Cl, or F;
R^{4N} is —CD₃, methyl, ethyl, or isopropyl; and
R^{4b} is H, halo, methyl, ethyl, or isopropyl.

In one embodiment of the compound of formula (D-I),
X² is N;
R^{4c} is cyano, —CF₃, Cl, or F;
R^{4N} is —CD₃, methyl, ethyl, or isopropyl; and
R^{4b} is H, halo, methyl, ethyl, or isopropyl.

In one embodiment of the compound of formula (D-I), the compound is

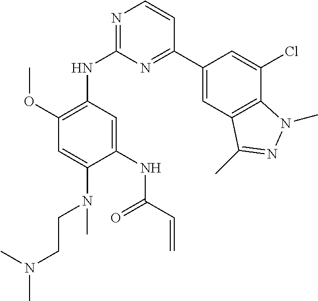

or a stereoisomer or a pharmaceutically acceptable salt, solvate, N-oxide, ester, or prodrug thereof.

In one embodiment, the present disclosure relates to compounds of formula (E):

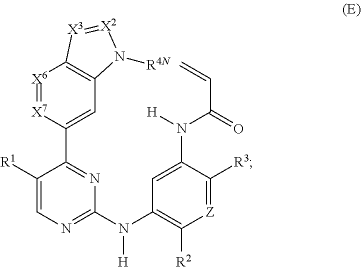

or a stereoisomer or a pharmaceutically acceptable salt, solvate, ester, or prodrug thereof;
wherein,
Z is CH or N;
X², X³, X⁶ and X⁷ are each CH, CR⁴, or N;
R¹ is hydrogen, fluoro, chloro, bromo, methyl, ethyl, hydroxyl, methoxy, ethoxy, isopropoxy, cyclopropoxy, —OCF₃, —OCH₂CF₃, —OCH₂CHF₂, ethenyl, ethynyl, CF₃, CHF₂, CHO, CH₂OH, CONH₂, CO₂Me, CONHMe, CONMe₂, or cyano;
R² is —OCF₃, —OCHF₂, —OCF₂CF₃, —OCH₂CHF₂, —OCH₂CF₃, cyclopropyl, cyclopropoxy, methoxy, —OCD₃, ethoxy, or isopropoxy;
R³ is N(R¹⁰)C₂₋₆ alkyl-NR¹⁰R¹⁰;
each R⁴ is independently H, cyano, halo, —C₁₋₆ alkyl, —C₁₋₆ haloalkyl, carboxy-C₁₋₆ alkyl, —C₁₋₆ hydroxyalkyl, R⁸R⁹N—C₁₋₆ alkyl-, —C₂₋₆ alkenyl, —C₂₋₆ alkynyl, C₁₋₆ acyl-, R⁷—(CH₂)ₚC(═O)—, C₁₋₆ hydroxyalkyl-C(═O)—, carboxy, —C₁₋₆ alkoxycarbonyl, —C(═O)NR⁸R⁹, hydroxyl, alkoxy, C₁₋₆ acyloxy, —NR⁸R⁹, C₁₋₆ acyl-N(R¹⁰)—, or R⁷SO₂—; and
R⁸ and R⁹ are independently H, —CD₃, C₁₋₆ alkyl, C₃₋₆ alkenyl, C₃₋₆ alkynyl, C₃₋₈ cycloalkyl, C₃₋₈ cycloalkenyl, C₁-C₆ acyl, 4-12 membered monocyclic or bicyclic heterocyclyl, 4-12 membered monocyclic or bicyclic heterocyclyl-C₁-C₆ alkyl-, C₆-C₁₂ aryl, 5-12 membered heteroaryl; and R⁸ and R⁹ may be further independently substituted with up to three substituents chosen from hydroxyl, C₁₋₆ alkoxy, C₁₋₆ hydroxyalkylC₂₋₆ hydroxyalkoxy, oxo, thiono, cyano or halo; or
alternatively, R⁸ and R⁹, taken together with the N atom to which they are both attached, form a heterocyclic ring of 4-7 members, containing up to one other heteroatom selected from O, S, or NR¹¹, or a heterobicyclic ring of 7-12 members which may be fused, bridged or spiro, and contain up to two other heteroatoms chosen from O, S(O)ₓ, or NR¹¹, and these heterocyclic rings are optionally substituted with up to three substituents chosen from hydroxyl, C₁₋₆ alkoxy, C₁₋₆ hydroxyalkyl, C₁₋₆ alkoxy-C₁₋₆ alkyl, C₁₋₆ alkoxy-C₁₋₆ alkoxy, C₂₋₆ hydroxyalkoxy, oxo, thiono, cyano or halo;
R^{4N} is H, —CD₃, or —C₁₋₆ alkyl;
R⁷ is OH, NR⁸R⁹, —O(CH₂)_{q}NR⁸R⁹, C₁₋₆ alkoxy, or C₂₋₆ hydroxyalkoxy;
each R¹⁰ is independently H, —CD₃, C₁₋₆ alkyl, C₃₋₆ cycloalkyl, C₂₋₆ hydroxyalkyl, C₁₋₆ alkoxy-C₁₋₆ alkyl or C₂₋₆ alkyl-NR⁸R⁹;
p=0, 1, 2, 3, or 4;
q=2, 3, or 4; and
x=0, 1, or 2.

In one embodiment, the present disclosure relates to compounds of formula (F) or (G):

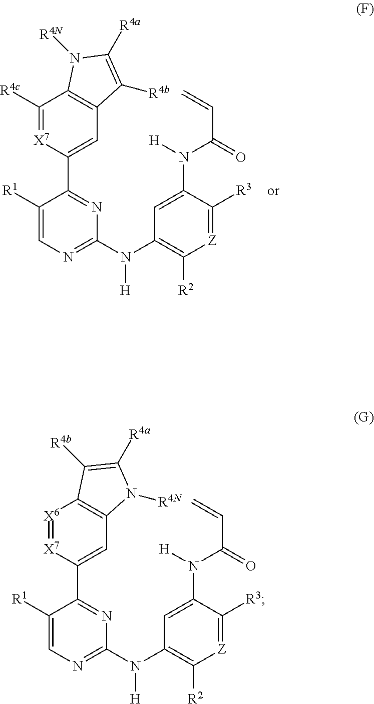

or a stereoisomer or a pharmaceutically acceptable salt, solvate, ester, or prodrug thereof;
wherein,
Z is CH or N;
X⁶ and X⁷ are each CH, CR⁴, or N;
R¹ is independently selected from hydrogen, fluoro, chloro, bromo, methyl, ethyl, hydroxyl, methoxy, ethoxy, isopropoxy, cyclopropoxy, —OCF₃, —OCH₂CF₃, —OCH₂CHF₂, ethenyl, ethynyl, CF₃, CHF₂, CHO, CH₂OH, CONH₂, CO₂Me, CONHMe, CONMe₂, and cyano;
R² is —OCF₃, —OCHF₂, —OCF₂CF₃, —OCH₂CHF₂, —OCH₂CF₃, cyclopropyl, cyclopropoxy, methoxy, —OCD₃, ethoxy, or isopropoxy;
R³ is N(R¹⁰)C₂₋₆ alkyl-NR¹⁰R¹⁰;
each R⁴ is independently H, cyano, halo, —C₁₋₆ alkyl, —C₁₋₆ haloalkyl, carboxy-C₁₋₆ alkyl, —C₁₋₆ hydroxyalkyl, R⁸R⁹N—C₁₋₆ alkyl-, —C₂₋₆ alkenyl, —C₂₋₆ alkynyl, C₁₋₆ acyl-, R⁷—(CH₂)ₚC(═O)—, C₁₋₆ hydroxyalkyl-C(═O)—, carboxy, —C₁₋₆ alkoxycarbonyl, —C(═O)NR⁸R⁹, hydroxyl, alkoxy, C₁₋₆ acyloxy, —NR⁸R⁹, C₁₋₆ acyl-N(R¹⁰)—, R⁷SO₂—,
R^{4a} and R^{4b} are each independently H, halo, —C₁₋₆ alkyl, or —C₁₋₆ haloalkyl;
R^{4c} is cyano, C₁₋₆ acyl-, —C(═O)NR⁸R⁹, hydroxyl, alkoxy, or F;
R^{4N} is H, —CD₃, —C₁₋₆ alkyl, or —C₁₋₆ haloalkyl;
R⁷ is OH, NR⁸R⁹, O(CH₂)_{q}NR⁸R⁹, C₁₋₆ alkoxy, or C₂₋₆ hydroxyalkoxy;
R⁸ and R⁹ are independently H, —CD₃, C₁₋₆ alkyl, C₃₋₆ alkenyl, C₃₋₆ alkynyl, C₃₋₈ cycloalkyl, C₃₋₈ cycloalkenyl, C₁-C₆ acyl, 4-12 membered monocyclic or bicyclic heterocyclyl, 4-12 membered monocyclic or bicyclic heterocyclyl-C₁-C₆ alkyl-, C₆-C₁₂ aryl, 5-12 membered heteroaryl; and R⁸ and R⁹ may be further independently substituted with up to three substituents chosen from hydroxyl, C₁₋₆ alkoxy, C₁₋₆ hydroxyalkylC₂₋₆ hydroxyalkoxy, oxo, thiono, cyano or halo; or
each R¹⁰ is independently H, —CD₃, C₁₋₆ alkyl, C₃₋₆ cycloalkyl, C₂₋₆ hydroxyalkyl, C₁₋₆ alkoxy-C₁₋₆ alkyl or C₂₋₆ alkyl-NR⁸R⁹; or
p=0, 1, 2, 3, or 4; and
q=2, 3, or 4.

In one embodiment, the compound of formula (D), (D-I), (E), (E-I), (F), or (G) is not:

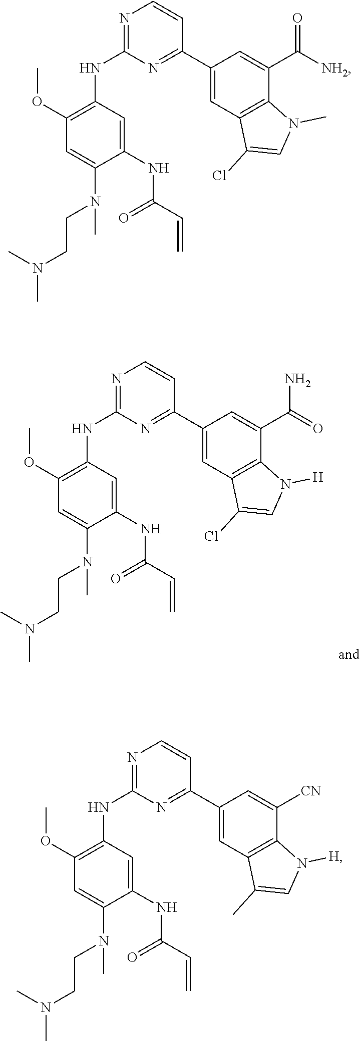

or a stereoisomer or a pharmaceutically acceptable salt, solvate, ester, or prodrug thereof.

In one embodiment, the compound of formula (D), (D-I), (E), (E-I), (F), or (G) is:

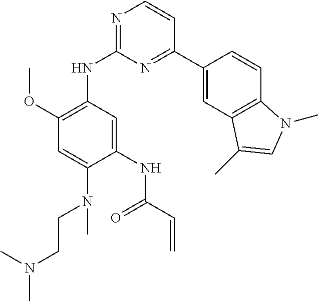

or a stereoisomer or a pharmaceutically acceptable salt, solvate, ester, or prodrug thereof.

In one embodiment, the compound of formula (D), (D-I), (E), (E-I), (F), or (G) is:

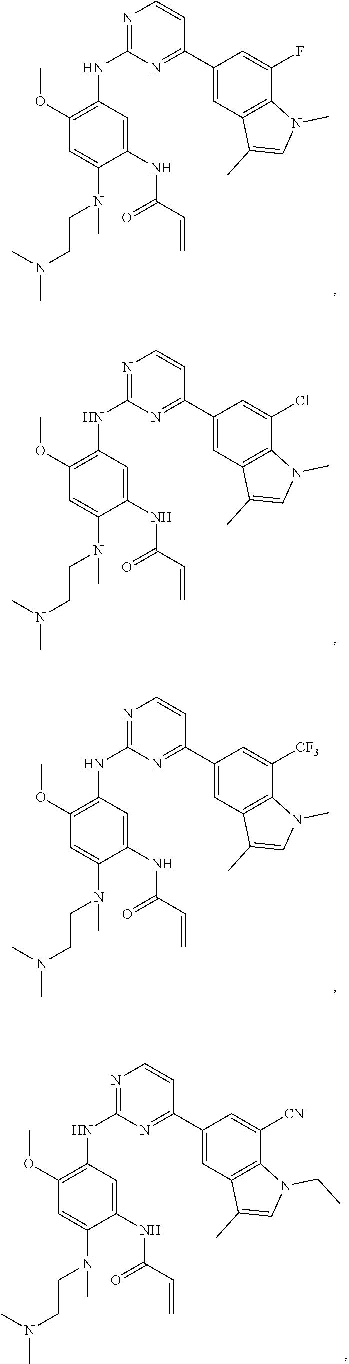

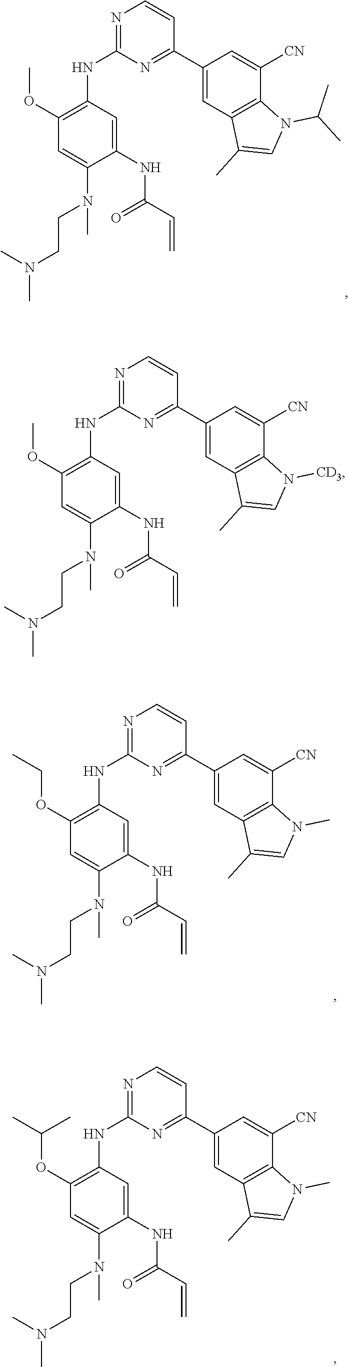

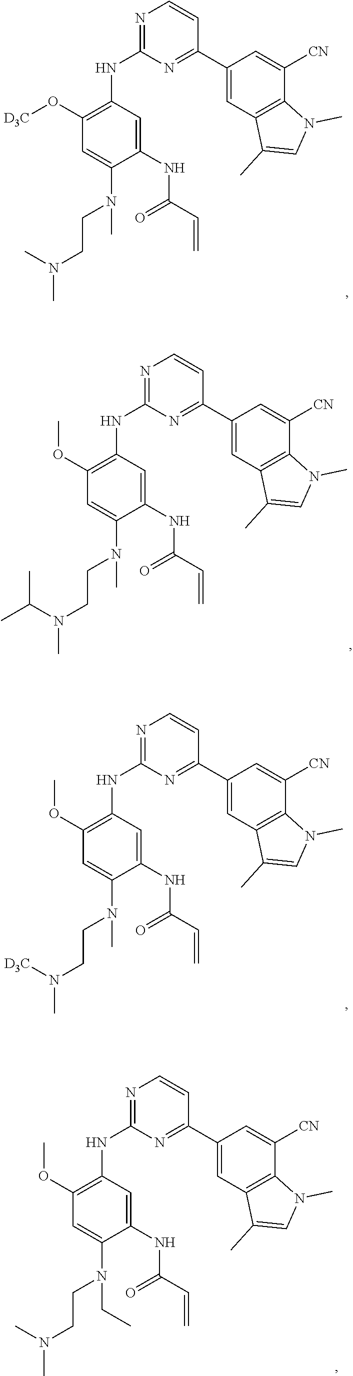

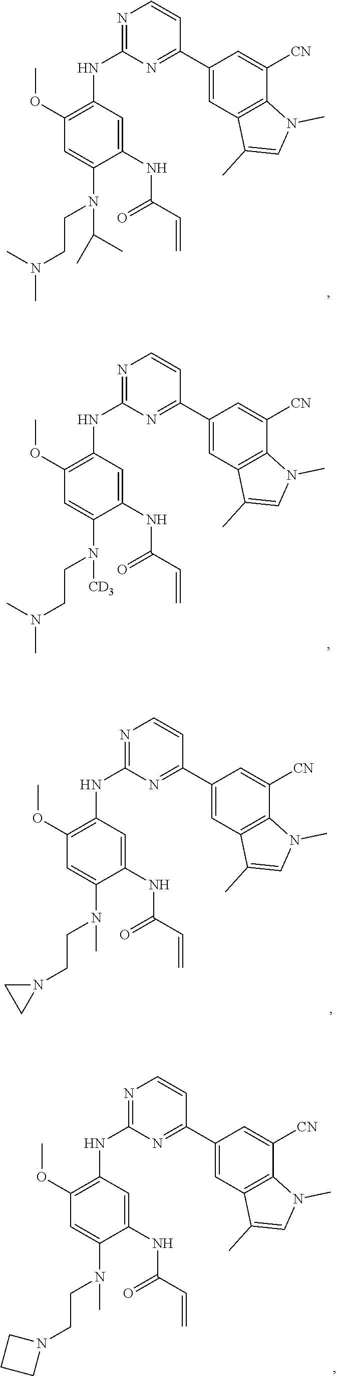

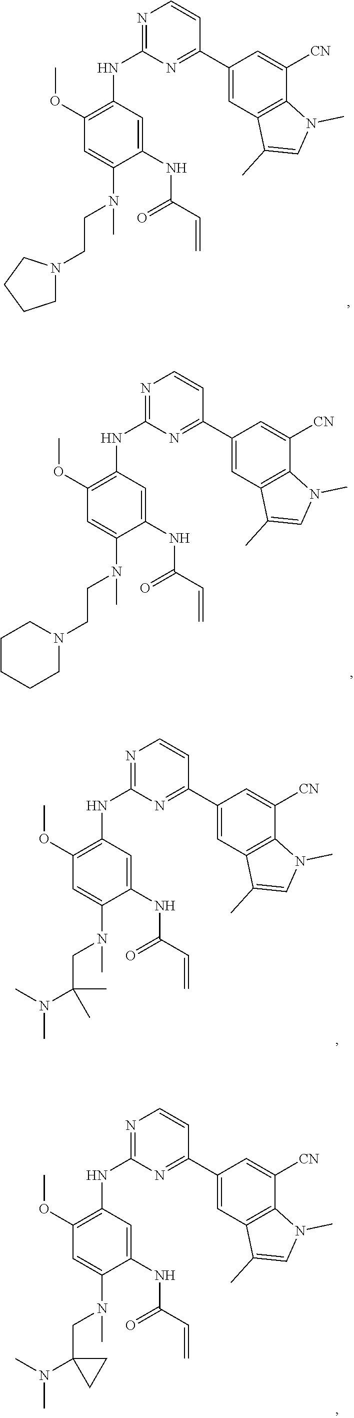

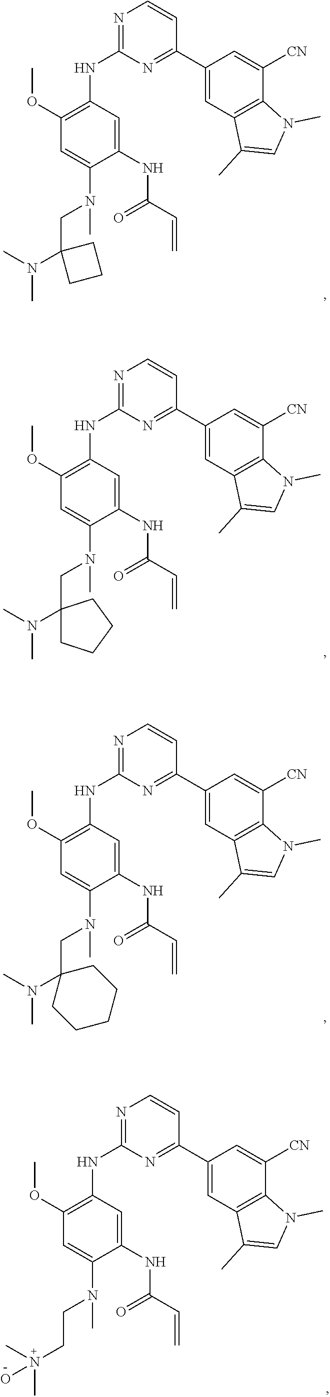

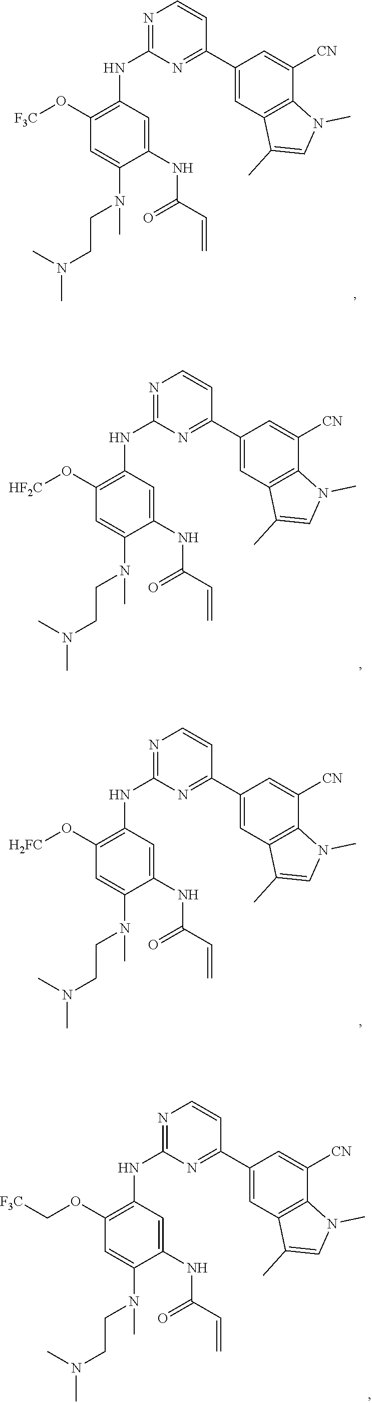

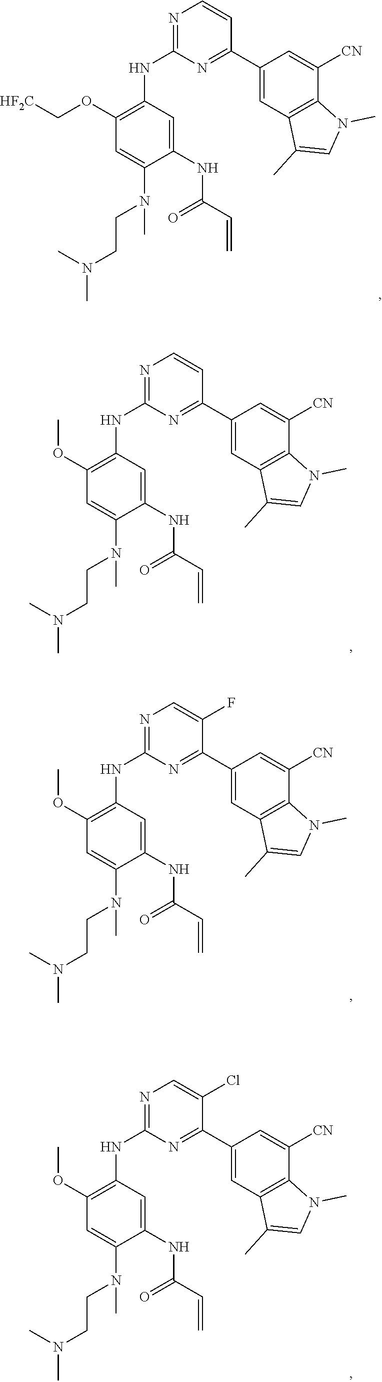

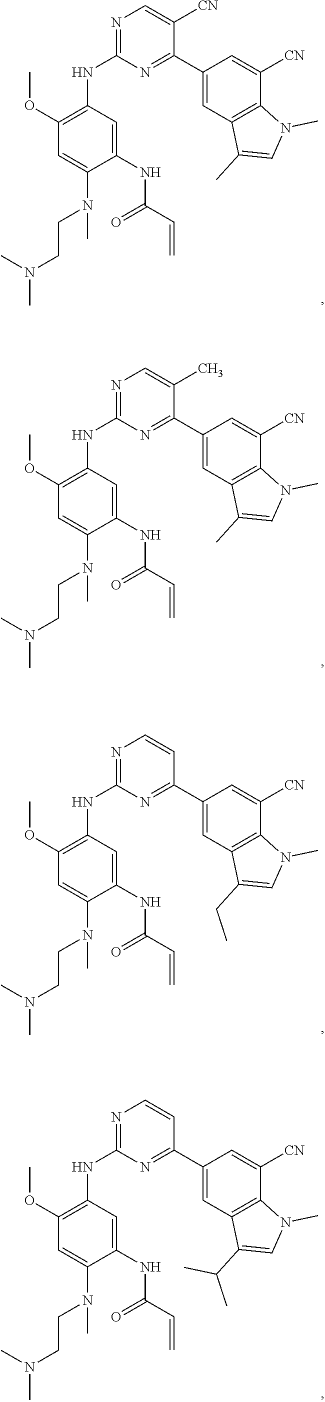

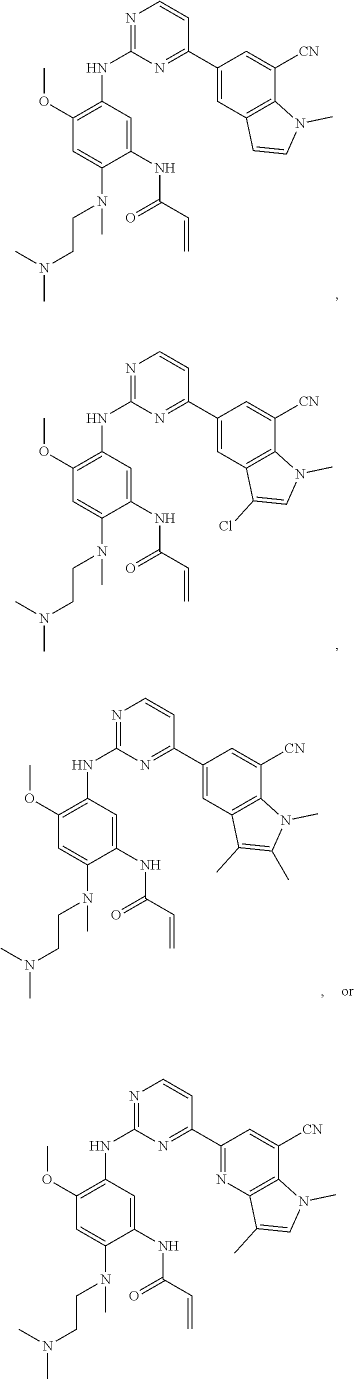

or a stereoisomer or a pharmaceutically acceptable salt, solvate, N-oxide, ester, or prodrug thereof.

In one embodiment, the present disclosure relates to compounds of formula (E-I):

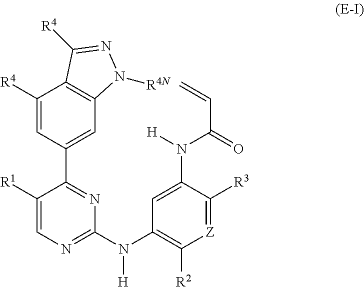

or a stereoisomer or a pharmaceutically acceptable salt, solvate, N-oxide, ester, or prodrug thereof, wherein,
Z is CH or N;
R¹ is hydrogen, fluoro, chloro, bromo, methyl, ethyl, hydroxyl, methoxy, ethoxy, isopropoxy, cyclopropoxy, —OCF₃, —OCH₂CF₃, —OCH₂CHF₂, ethenyl, ethynyl, CF₃, CHF₂, CHO, CH₂OH, CONH₂, CO₂Me, CONHMe, CONMe₂, or cyano;
R² is —OCF₃, —OCHF₂, —OCF₂CF₃, —OCH₂CHF₂, —OCH₂CF₃, cyclopropyl, cyclopropoxy, methoxy, —OCD₃, ethoxy, or isopropoxy;
R³ is N(R¹⁰)C₂₋₆ alkyl-NR¹⁰R¹⁰ or —N(R¹⁰)(C₃₋₁₀ cycloalkylalkyl)-NR¹⁰R¹⁰;
each R⁴ is independently H, cyano, halo, —C₁₋₆ alkyl, —C₁₋₆ haloalkyl, carboxy-C₁₋₆ alkyl, —C₁₋₆ hydroxyalkyl, R⁸R⁹N—C₁₋₆ alkyl-, —C₂₋₆ alkenyl, —C₂₋₆ alkynyl, C₁₋₆ acyl-, R⁷—(CH₂)ₚC(═O)—, C₁₋₆ hydroxyalkyl-C(═O)—, carboxy, —C₁₋₆ alkoxycarbonyl, —C(═O)NR⁸R⁹, hydroxyl, alkoxy, C₁₋₆ acyloxy, —NR⁸R⁹, C₁₋₆ acyl-N(R¹⁰)—, or R⁷SO₂—; and
R⁸ and R⁹ are independently H, —CD₃, C₁₋₆ alkyl, C₃₋₆ alkenyl, C₃₋₆ alkynyl, C₃₋₈ cycloalkyl, C₃₋₈ cycloalkenyl, C₁-C₆ acyl, 4-12 membered monocyclic or bicyclic heterocyclyl, 4-12 membered monocyclic or bicyclic heterocyclyl-C₁-C₆ alkyl-, C₆-C₁₂ aryl, 5-12 membered heteroaryl; and R⁸ and R⁹ may be further independently substituted with up to three substituents chosen from hydroxyl, C₁₋₆ alkoxy, C₁₋₆ hydroxyalkylC₂₋₆ hydroxyalkoxy, oxo, thiono, cyano or halo; or
alternatively, R⁸ and R⁹, taken together with the N atom to which they are both attached, form a heterocyclic ring of 4-7 members, containing up to one other heteroatom selected from O, S, or NR¹¹, or a heterobicyclic ring of 7-12 members which may be fused, bridged or spiro, and contain up to two other heteroatoms chosen from O, S(O)ₓ, or NR¹¹, and these heterocyclic rings are optionally substituted with up to three substituents chosen from hydroxyl, C₁₋₆ alkoxy, C₁₋₆ hydroxyalkyl, C₁₋₆ alkoxy-C₁₋₆ alkyl, C₁₋₆ alkoxy-C₁₋₆ alkoxy, C₂₋₆ hydroxyalkoxy, oxo, thiono, cyano or halo;
R^{4N} is H, —CD₃, or —C₁₋₆ alkyl;
R⁷ is OH, —NR⁸R⁹, —O(CH₂)_{q}NR⁸R⁹, C₁₋₆ alkoxy, or C₂₋₆ hydroxyalkoxy;
each R¹⁰ is independently H, —CD₃, C₁₋₆ alkyl, C₃₋₆ cycloalkyl, C₂₋₆ hydroxyalkyl, C₁₋₆ alkoxy-C₁₋₆ alkyl or C₂₋₆ alkyl-NR⁸R⁹;
alternatively, two R¹⁰ on the same N atom, taken together form a heterocyclic ring of 3-7 members, optionally substituted with up to three substituents chosen from hydroxyl, C₁₋₆ alkoxy, C₁₋₆ hydroxyalkyl, C₁₋₆ alkoxy-C₁₋₆ alkyl, C₁₋₆ alkoxy-C₁₋₆ alkoxy, C₂₋₆ hydroxyalkoxy, oxo, thiono, cyano or halo;
p=0, 1, 2, 3, or 4;
q=2, 3, or 4; and
x=0, 1, or 2.

In some embodiments of the compound of formula (E-I),
R³ is N(R¹⁰)C₂₋₆ alkyl-NR¹⁰R¹⁰ or —N(R¹⁰)(C₃₋₁₀ cycloalkylalkyl)-NR¹⁰R¹⁰;
each R⁴ is independently H, cyano, halo, —C₁₋₆ alkyl, or —C₁₋₆ haloalkyl; and
R^{4N} is H, —CD₃, or —C₁₋₆ alkyl; and
each R¹⁰ is independently H, —CD₃, or —C₁₋₆ alkyl.

In some embodiments of the compound of formula (E-I), the compound is

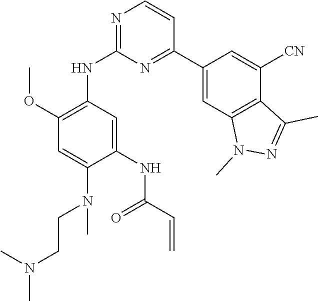

or a stereoisomer or a pharmaceutically acceptable salt, solvate, N-oxide, ester, or prodrug thereof.

In some embodiments, the present disclosure relates to compounds of formula (H)

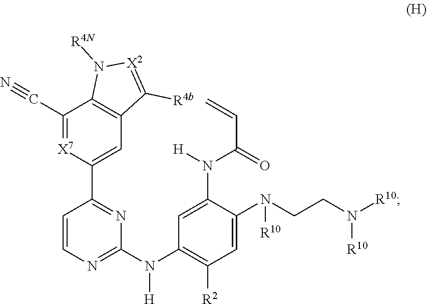

or a stereoisomer or a pharmaceutically acceptable salt, solvate, ester, or prodrug thereof;
wherein,
X⁷ is CH or N;
X² is independently CH, CCH₃, or N;
R² is methoxy, —OCD₃, ethoxy, or isopropoxy;
R^{4b} is H, F, Cl, or CH₃;
R^{4N} is H, —CD₃, CH₃, Et, or CH(CH₃)₂; and
each R¹⁰ is independently H, —CD₃, —CH₃, —CH₂CH₃, or —CH(CH₃)₂.

In one embodiment, the compound of structure (H) comprises
X⁷ is CH or N;
X² is independently CH or CCH₃;
R² is methoxy, —OCD₃, ethoxy, or isopropoxy;
R^{4b} is H, F, Cl, or CH₃;
R^{4N} is H, —CD₃, CH₃, Et, or CH(CH₃)₂; and
each R¹⁰ is independently H, —CD₃, —CH₃, —CH₂CH₃, or —CH(CH₃)₂.

In one embodiment, the compound of the present disclosure has the structure of formula (H-I)

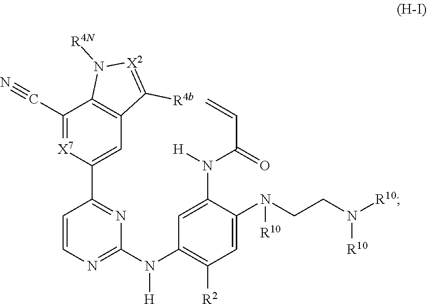

or a stereoisomer or a pharmaceutically acceptable salt, solvate, ester, or prodrug thereof;
wherein,
X⁷ is CH or N;
X² is independently CH, CCH₃, or N;
R² is methoxy, —OCD₃, ethoxy, or isopropoxy;
R^{4b} is H, F, Cl, or CH₃;
R^{4N} is H, —CD₃, CH₃, Et, or CH(CH₃)₂; and
each R¹⁰ is independently —CD₃, —CH₃, —CH₂CH₃, or —CH(CH₃)₂.

In one embodiment, the compound of formula (H-I) comprises:
X⁷ is CH;
X² is independently CH or CCH₃;
R² is methoxy, —OCD₃, ethoxy, or isopropoxy;
R^{4b} is H, F, Cl, or CH₃;
R^{4N} is H, —CD₃, CH₃, Et, or CH(CH₃)₂; and
each R¹⁰ is independently —CD₃, —CH₃, —CH₂CH₃, or —CH(CH₃)₂.

In one embodiment, the compound of structure (H) is

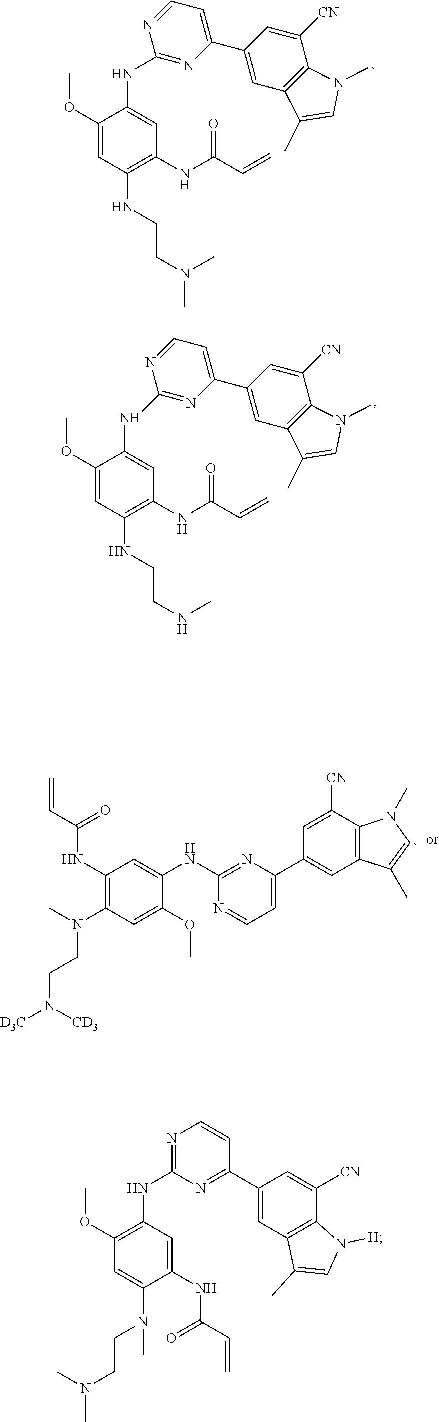

or a stereoisomer or a pharmaceutically acceptable salt, solvate, ester, or prodrug thereof.

In one embodiment, the compound of structure (H) or (H-I) is:

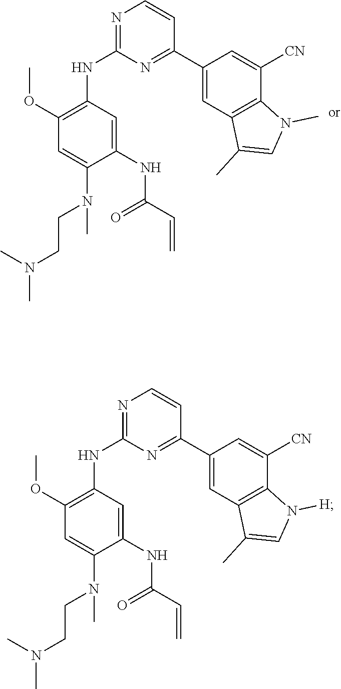

or a stereoisomer or a pharmaceutically acceptable salt, solvate, ester, or prodrug thereof. In one embodiment, the compound of structure (H) or (H-I) is:

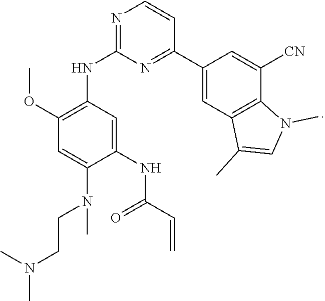

In another embodiment, the present disclosure relates to compounds of formula (J):

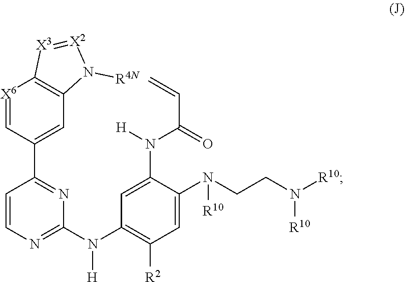

or a stereoisomer or a pharmaceutically acceptable salt, solvate, ester, or prodrug thereof;
wherein,
X⁶ is N or C—R⁴, wherein R⁴ is H, cyano, CONH₂, CONHCH₃, CON(CH₃)₂, COCH₃;
X² is independently C—H, C—CH₃ or N;
X³ is independently C—H, C—CH₃, C—CF₃, C—CHF₂, C—F, C—Cl, or N;
R^{4N} is H, —CD₃, —CH₃, —CH₂CH₃, or —CH(CH₃)₂;
R² is —OCF₃, —OCHF₂, —OCF₂CF₃, —OCH₂CHF₂, —OCH₂CF₃, cyclopropyl, cyclopropoxy, methoxy, —OCD₃, ethoxy, or isopropoxy;
each R¹⁰ is independently H, —CD₃, C₁₋₆ alkyl, C₃₋₆ cycloalkyl, C₂₋₆ hydroxyalkyl, C₁₋₆ alkoxy-C₁₋₆ alkyl or C₂₋₆ alkyl-NR⁸R⁹; and
R⁸ and R⁹ are independently H, —CD₃, C₁₋₆ alkyl, C₃₋₆ alkenyl, C₃₋₆ alkynyl, C₃₋₈ cycloalkyl, C₃₋₈ cycloalkenyl, C₁-C₆ acyl, 4-12 membered monocyclic or bicyclic heterocyclyl, 4-12 membered monocyclic or bicyclic heterocyclyl-C₁-C₆ alkyl-, C₆-C₁₂ aryl, 5-12 membered heteroaryl; and R⁸ and R⁹ may be further independently substituted with up to three substituents chosen from hydroxyl, C₁₋₆ alkoxy, C₁₋₆ hydroxyalkylC₂₋₆ hydroxyalkoxy, oxo, thiono, cyano or halo.

In one embodiment, the compound of formula (J) comprises:
X⁶ is C—CN;
X² is C—H or C—CH₃;
X³ is C—H or C—CH₃;
R^{4N} is H, —CD₃, —CH₃, —CH₂CH₃, or —CH(CH₃)₂;
R² is methoxy, —OCD₃, ethoxy, or isopropoxy;
each R¹⁰ is independently H, —CD₃, —CH₃, —CH₂CH₃, or —CH(CH₃)₂.

In one embodiment, the compound of formula (J) is:

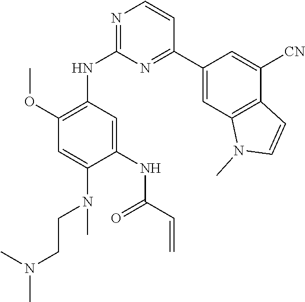 [SMILES: C=CC(=O)Nc1cc(Nc2nccc(-c3cc(C#N)c4ccn(C)c4c3)n2)c(OC)cc1N(C)CCN(C)C]

or a stereoisomer or a pharmaceutically acceptable salt, solvate, ester, or prodrug thereof. In one embodiment, the compound of formula (J) is:

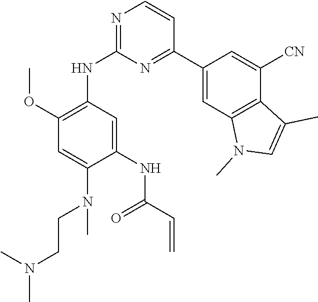

or a stereoisomer or a pharmaceutically acceptable salt, solvate, ester, or prodrug thereof.

In one embodiment, the present disclosure relates to compounds of formula (K):

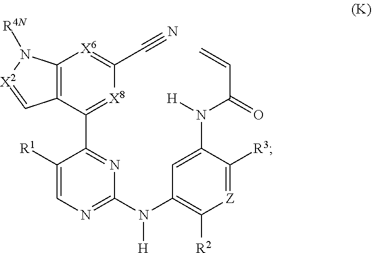

or a stereoisomer or a pharmaceutically acceptable salt, solvate, ester, or prodrug thereof;
wherein,
Z is CH or N;
X² is CR^{4a} or N;
X⁶ is CR⁴b or N;
X⁸ is CH or N;
R¹ is hydrogen, methyl, fluoro, chloro, bromo, CF₃, or cyano;
R² is —OCF₃, —OCHF₂, —OCF₂CF₃, —OCH₂CHF₂, —OCH₂CF₃, cyclopropoxy, methoxy, —OCD₃, ethoxy, or isopropoxy;
R³ is N(R¹⁰)C₂₋₆ alkyl-NR¹⁰R¹⁰;
R^{4a} is H, cyano, halo, —C₁₋₆ alkyl, or —C₁₋₆ haloalkyl;
R^{4b} is H, cyano, nitro, halo, —C₁₋₆ alkyl, —C₁₋₆ haloalkyl, carboxy-C₁₋₆ alkyl, —C₁₋₆ hydroxyalkyl, R⁸R⁹N—C₁₋₆ alkyl-, —C₂₋₆ alkenyl, —C₂₋₆ alkynyl, C₁₋₆ acyl-, R⁷—(CH₂)ₚC(═O)—, C₁₋₆ hydroxyalkyl-C(═O)—, carboxy, —C₁₋₆ alkoxycarbonyl, —C(═O)NR⁸R⁹, hydroxyl, alkoxy, —OCD₃, C₁₋₆ acyloxy, —NR⁸R⁹, C₁₋₆ acyl-N(R¹⁰)—, or R⁷SO₂—;
R^{4N} is H, —C₁₋₆ alkyl, or —CD₃;
R⁸ and R⁹ are independently H, —CD₃, C₁₋₆ alkyl, C₃₋₈ cycloalkyl, C₃₋₈ cycloalkyl-(C₁₋₃ alkyl)-, C₁-C₆ acyl, phenyl, monocyclic heteroaryl, or monocyclic heterocyclyl; and R⁸ and R⁹ may be further independently substituted with up to three substituents chosen from hydroxyl, C₁₋₆ alkoxy, oxo, thiono, cyano or halo; or
alternatively, R⁸ and R⁹, taken together with the N atom to which they are both attached, form a heterocyclic ring of 4-7 members, containing up to one other heteroatom selected from O, S, or NR¹¹, or a heterobicyclic ring of 7-12 members which may be fused, bridged or spiro, and contain up to two other heteroatoms chosen from O, S(O)ₓ, or NR¹¹, and these heterocyclic rings are optionally substituted with up to three substituents chosen from hydroxyl, C₁₋₆ alkoxy, C₁₋₆ hydroxyalkyl, C₁₋₆ alkoxy-C₁₋₆ alkyl, C₁₋₆ alkoxy-C₁₋₆ alkoxy, C₂₋₆ hydroxyalkoxy, oxo, thiono, cyano or halo;
each R¹⁰ is independently H, —CD₃, C₁₋₆ alkyl, C₃₋₆ cycloalkyl, C₂₋₆ hydroxyalkyl, C₁₋₆ alkoxy-C₁₋₆ alkyl or C₂₋₆ alkyl-NR⁸R⁹;
p=0, 1, 2, 3, or 4;
q=2, 3, or 4; and
x=0, 1, or 2.

In another embodiment, the present disclosure relates to compounds of formula (L):

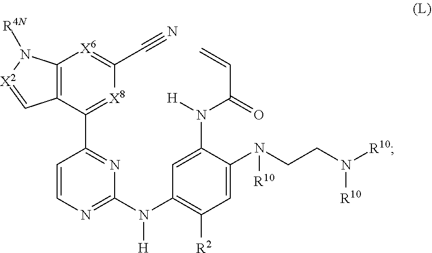

or a stereoisomer or a pharmaceutically acceptable salt, solvate, ester, or prodrug thereof;
wherein,
X² is CR^{4a} or N;
X⁶ is CR⁴b or N;
X⁸ is CH or N;
R² is methoxy, —OCD₃, ethoxy, or isopropoxy;
R^{4a} is H, cyano, halo, —C₁₋₆ alkyl, or —C₁₋₆ haloalkyl;
R^{4b} is H, cyano, nitro, halo, —C₁₋₆ alkyl, —C₁₋₆ haloalkyl, carboxy-C₁₋₆ alkyl, —C₁₋₆ hydroxyalkyl, R⁸R⁹N—C₁₋₆ alkyl-, —C₂₋₆ alkenyl, —C₂₋₆ alkynyl, C₁₋₆ acyl-, R⁷—(CH₂)ₚC(═O)—, C₁₋₆ hydroxyalkyl-C(═O)—, carboxy, —C₁₋₆ alkoxycarbonyl, —C(═O)NR⁸R⁹, hydroxyl, alkoxy, —OCD₃, C₁₋₆ acyloxy, —NR⁸R⁹, C₁₋₆ acyl-N(R¹⁰)—, R⁷SO₂—;
R^{4N} is H, —CH₃, Et, CH(CH₃)₂, or —CD₃;
R⁸ and R⁹ are independently H, —CD₃, C₁₋₆ alkyl, C₃₋₈ cycloalkyl, C₃₋₈ cycloalkyl-(C₁₋₃ alkyl)-, C₁-C₆ acyl, phenyl, monocyclic heteroaryl, or monocyclic heterocyclyl; and R⁸ and R⁹ may be further independently substituted with up to three substituents chosen from hydroxyl, C₁₋₆ alkoxy, oxo, thiono, cyano or halo; or
alternatively, R⁸ and R⁹, taken together with the N atom to which they are both attached, form a heterocyclic ring of 4-7 members, containing up to one other heteroatom selected from O, S, or NR¹¹, or a heterobicyclic ring of 7-12 members which may be fused, bridged or spiro, and contain up to two other heteroatoms chosen from O, S(O)ₓ, or NR¹¹, and these heterocyclic rings are optionally substituted with up to three substituents chosen from hydroxyl, C₁₋₆ alkoxy, C₁₋₆ hydroxyalkyl, C₁₋₆ alkoxy-C₁₋₆ alkyl, C₁₋₆ alkoxy-C₁₋₆ alkoxy, C₂₋₆ hydroxyalkoxy, oxo, thiono, cyano or halo;
each R¹⁰ is independently H, —CD₃, C₁₋₆ alkyl, C₃₋₆ cycloalkyl, C₂₋₆ hydroxyalkyl, C₁₋₆ alkoxy-C₁₋₆ alkyl or C₂₋₆ alkyl-NR⁸R⁹;
p=0, 1, 2, 3, or 4;
q=2, 3, or 4; and
x=0, 1, or 2.

In another embodiment, the compound of formula (L) comprises:
X² is CR^{4a} or N;
X⁶ is CR⁴b or N;
X⁸ is CH or N;
R² is methoxy, —OCD₃, ethoxy, or isopropoxy;
R^{4a} is H, F, Cl, CH₃, CF₃, or CHF₂;
R^{4b} is H, cyano, nitro, halo, —C₁₋₆ alkyl, or —C₁₋₆ haloalkyl;
R^{4N} is H, —CD₃, —CH₃, —CH₂CH₃, or —CH(CH₃)₂; and
each R¹⁰ is independently H, —CD₃, —CH₃, —CH₂CH₃, or —CH(CH₃)₂.

In some embodiments, the compound of formula (L) comprises:
X² is CR^{4a} or N;
X⁶ is CR⁴b;
X⁸ is CH;
R² is methoxy, —OCD₃, ethoxy, or isopropoxy;
R^{4a} is H, F, CH₃, CF₃, or CHF₂;
R^{4b} is H, CH₃, F, Cl, CF₃, or CHF₂;
R^{4N} is H, —CD₃, —CH₃, —CH₂CH₃, or —CH(CH₃)₂;
each R¹⁰ is independently H, —CD₃, —CH₃, —CH₂CH₃, or —CH(CH₃)₂.

In one embodiment, the compound of formula (L) is:

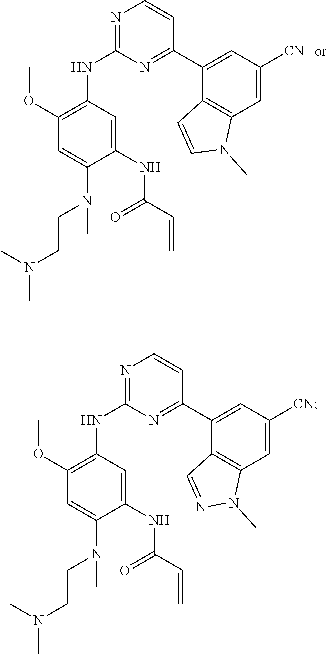 [SMILES: C=CC(=O)Nc1cc(Nc2nccc(-c3cc(C#N)cc4c3ccn4C)n2)c(OC)cc1N(C)CCN(C)C ; C=CC(=O)Nc1cc(Nc2nccc(-c3cc(C#N)cc4c3cnn4C)n2)c(OC)cc1N(C)CCN(C)C]

or a stereoisomer or a pharmaceutically acceptable salt, solvate, ester, or prodrug thereof.

In some embodiments, the present disclosure relates to compounds of formula (M):

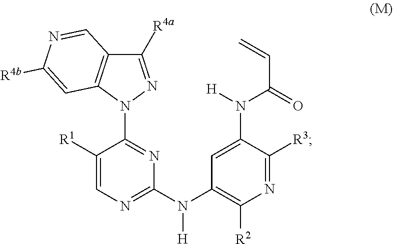

or a stereoisomer or a pharmaceutically acceptable salt, solvate, ester, or prodrug thereof;
wherein,
Z is CH or N;
R¹ is hydrogen, methyl, fluoro, chloro, bromo, —CF₃, or cyano;
R² is —OCF₃, —OCHF₂, —OCF₂CF₃, —OCH₂CHF₂, —OCH₂CF₃, cyclopropoxy, methoxy, —OCD₃, ethoxy, or isopropoxy;
R³ is N(R¹⁰)C₂₋₆ alkyl-NR¹⁰R¹⁰;
R^{4a} is cyano, —C₁₋₆ hydroxyalkyl, C₁₋₆ acyl-, pyrazole, 1,2,3-triazole, tetrazole, —C(═O)NR⁸R⁹, —NR⁸R⁹, C₁₋₆ acyl-N(R¹⁰)—, (C₁₋₃ alkyl)SO₂NH—, (C₁₋₆ alkyl)SO₂—, or R⁷SO₂—;
R^{4b} is H, cyano, halo, —C₁₋₆ alkyl, or —C₁₋₆ haloalkyl;
R⁷ is —OH or —NR⁸R⁹;
R⁸ and R⁹ are independently H, —CD₃, C₁₋₆ alkyl, C₃₋₈ cycloalkyl, C₃₋₈ cycloalkyl-(C₁₋₃ alkyl)-, C₁-C₆ acyl, phenyl, monocyclic heteroaryl, or monocyclic heterocyclyl; and R⁸ and R⁹ may be further independently substituted with up to three substituents chosen from hydroxyl, C₁₋₆ alkoxy, oxo, thiono, cyano or halo; or
alternatively, R⁸ and R⁹, taken together with the N atom to which they are both attached, form a heterocyclic ring of 4-7 members, containing up to one other heteroatom chosen from O, S, or NR¹¹,
each R¹⁰ is independently H, —CD₃, C₁₋₆ alkyl, C₃₋₆ cycloalkyl, C₂₋₆ hydroxyalkyl, C₂₋₆ alkyl-NR⁸R⁹;
alternatively, two R¹⁰ on the same N atom to which they are both attached, form a heterocyclic ring of 5-6 members, containing up to one other heteroatom selected from O, S, or NR¹¹; and
each R¹¹ is independently hydrogen or C₁-C₆ alkyl, which is optionally substituted with up to three substituents selected from hydroxyl, oxo, thiono, cyano and halo.

In another embodiment, the compound of formula (M) comprises:
Z is CH;
R¹ is hydrogen, methyl, fluoro, chloro, bromo, —CF₃, or cyano;
R² is methoxy, —OCD₃, ethoxy, or isopropoxy;
R³ is —N(CH₃)CH₂CH₂NR¹⁰R¹⁰;
R^{4a} is —NR⁸R⁹;
R^{4b} is H, CH₃, F, Cl, CF₃, or CHF₂;
R⁸ and R⁹ are independently H, —CD₃, C₁₋₆ alkyl, C₃₋₈ cycloalkyl, C₃₋₈ cycloalkyl-(C₁₋₃ alkyl)-, C₁-C₆ acyl, phenyl, monocyclic heteroaryl, or monocyclic heterocyclyl; and R⁸ and R⁹ may be further independently substituted with up to three substituents chosen from hydroxyl, C₁₋₆ alkoxy, oxo, thiono, cyano or halo; and
each R¹⁰ is independently H, —CD₃, —CH₃, —CH₂CH₃, or —CH(CH₃)₂.
   In one embodiment, the compound of formula (M) is:

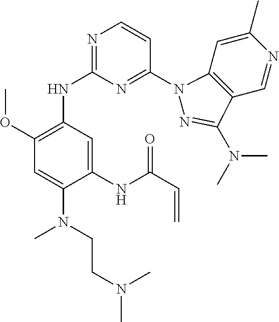 [SMILES: C=CC(=O)Nc1cc(Nc2nccc(-n3nc(N(C)C)c4cnc(C)cc43)n2)c(OC)cc1N(C)CCN(C)C]

or a stereoisomer or a pharmaceutically acceptable salt, solvate, ester, or prodrug thereof.

In another embodiment, the present disclosure relates to compounds of formula (N):

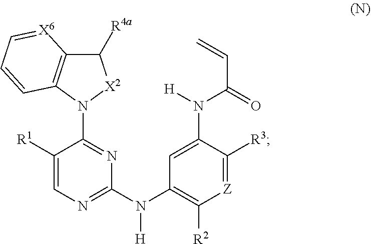

or a stereoisomer or a pharmaceutically acceptable salt, solvate, ester, or prodrug thereof;
wherein,
X² is CH, CCH₃, or N;
X⁶ is CR⁴ or N;
Z is CH or N;
R¹ is hydrogen, methyl, fluoro, chloro, bromo, —CF₃, or cyano;
R² is —OCF₃, —OCHF₂, —OCF₂CF₃, —OCH₂CHF₂, or —OCH₂CF₃;
R³ is N(R¹⁰)C₂₋₆ alkyl-NR¹⁰R¹⁰;
R⁴ is H, cyano, halo, —C₁₋₆ alkyl, —C₁₋₆ haloalkyl;
R^{4a} is independently cyano, —C₁₋₆ hydroxyalkyl, C₁₋₆ acyl-, pyrazole, 1,2,3-triazole, tetrazole, —C(═O)NR⁸R⁹, —NR⁸R⁹, C₁₋₆ acyl-N(R¹⁰)—, (C₁₋₃ alkyl)SO₂NH—, (C₁₋₆ alkyl)SO₂—, or R⁷SO₂—;
R⁷ is —OH or —NR⁸R⁹;
R⁸ and R⁹ are independently H, —CD₃, C₁₋₆ alkyl, C₃₋₈ cycloalkyl, C₃₋₈ cycloalkyl-(C₁₋₃ alkyl)-, C₁-C₆ acyl, phenyl, monocyclic heteroaryl, or monocyclic heterocyclyl; and R⁸ and R⁹ may be further independently substituted with up to three substituents chosen from hydroxyl, C₁₋₆ alkoxy, oxo, thiono, cyano or halo;
each R¹⁰ is independently H, —CD₃, C₁₋₆ alkyl, C₃₋₆ cycloalkyl, C₂₋₆ hydroxyalkyl, C₂₋₆ alkyl-NR⁸R⁹.

In one embodiment, the compounds of formula (N) have the structure of formula (O):

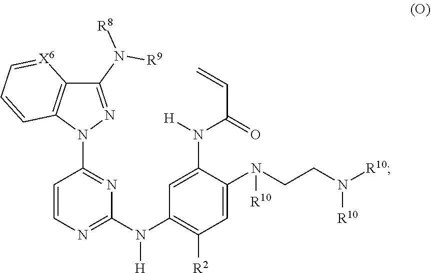

or a stereoisomer or a pharmaceutically acceptable salt, solvate, ester, or prodrug thereof;
wherein,
X⁶ is CH, CCH₃, or N;
R² is —OCF₃, —OCHF₂, —OCF₂CF₃, —OCH₂CHF₂, or —OCH₂CF₃;
R⁸ and R⁹ are each independently H, —CD₃, —CH₃, —CH₂CH₃, or —CH(CH₃)₂; and
each R¹⁰ is independently H, —CD₃, —CH₃, —CH₂CH₃, or —CH(CH₃)₂.

In other embodiments, the compound of formula (N) or (0) is:

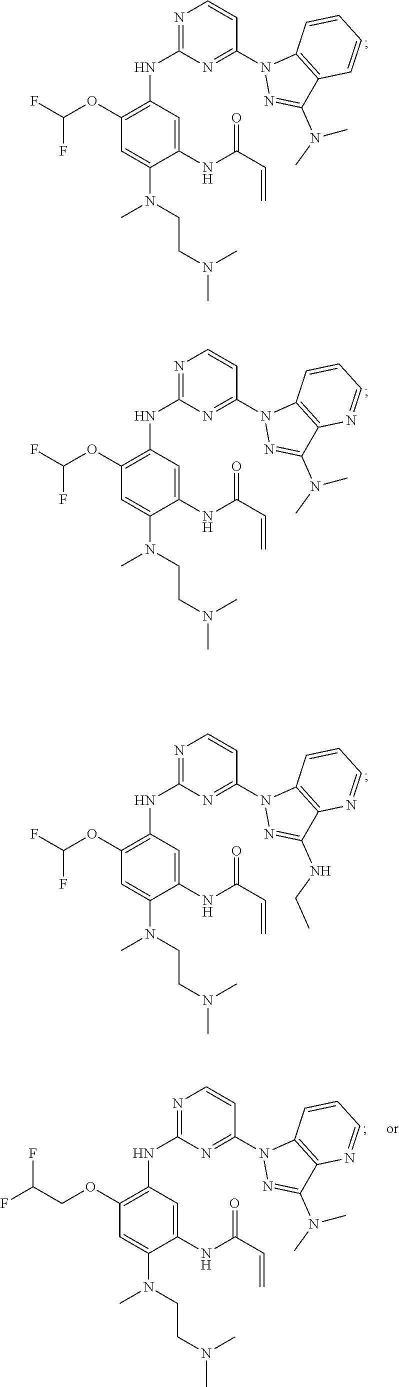 [SMILES: C=CC(=O)Nc1cc(Nc2nccc(-n3nc(N(C)C)c4ccccc43)n2)c(OC(F)F)cc1N(C)CCN(C)C ; C=CC(=O)Nc1cc(Nc2nccc(-n3nc(N(C)C)c4ncccc43)n2)c(OC(F)F)cc1N(C)CCN(C)C ; C=CC(=O)Nc1cc(Nc2nccc(-n3nc(NCC)c4ncccc43)n2)c(OC(F)F)cc1N(C)CCN(C)C ; C=CC(=O)Nc1cc(Nc2nccc(-n3nc(N(C)C)c4ncccc43)n2)c(OCC(F)F)cc1N(C)CCN(C)C]

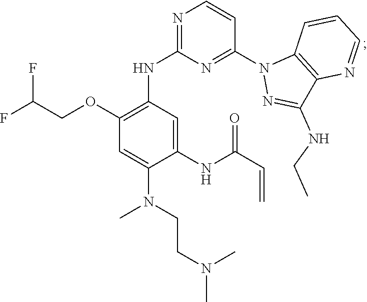 [SMILES: C=CC(=O)Nc1cc(Nc2nccc(-n3nc(NCC)c4ncccc43)n2)c(OCC(F)F)cc1N(C)CCN(C)C]

or a stereoisomer or a pharmaceutically acceptable salt, solvate, ester, or prodrug thereof.

In one embodiment, the present disclosure relates to compounds of formula (P):

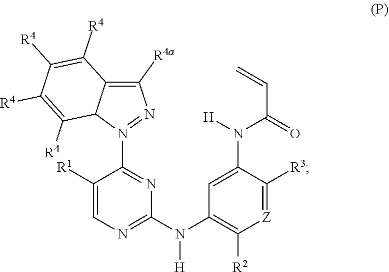

or a stereoisomer or a pharmaceutically acceptable salt, solvate, ester, tautomer, or prodrug thereof;
wherein:
Z is CH or N;
R¹ is independently selected from hydrogen, fluoro, chloro, bromo, methyl, ethyl, hydroxyl, methoxy, ethoxy, isopropoxy, cyclopropoxy, —OCF₃, —OCH₂CF₃, —OCH₂CHF₂, ethenyl, ethynyl, CF₃, CHF₂, CHO, CH₂OH, CONH₂, CO₂Me, CONHMe, CONMe₂, or cyano;
R² is —OCF₃, —OCHF₂, —OCF₂CF₃, —OCH₂CHF₂, —OCH₂CF₃, cyclopropyl, cyclopropoxy, methoxy, —OCD₃, ethoxy, or isopropoxy;
R³ is N(R¹⁰)C₂₋₆ alkyl-NR¹⁰R¹⁰, N(R¹⁰)C₂₋₆ alkyl-R⁷, O(CH₂)ₚR⁷, N(R¹⁰)C(═O)(CH₂)ₚR⁷ or R⁷;
each R⁴ is independently H, cyano, nitro, halo, —C₁₋₆ alkyl, —C₁₋₆ haloalkyl, carboxy-C₁₋₆ alkyl, —C₁₋₆ hydroxyalkyl, R⁸R⁹N—C₁₋₆ alkyl-, —C₂₋₆ alkenyl, —C₂₋₆ alkynyl, C₁₋₆ acyl-, R⁷⁻(CH₂)ₚC(═O)—, C₁₋₆ hydroxyalkyl-C(═O)—, carboxy, —C₁₋₆ alkoxycarbonyl, —C(═O)NR⁸R⁹, hydroxyl, alkoxy, C₁₋₆ acyloxy, —NR⁸R⁹, C₁₋₆ acyl-N(R¹⁰)—, or R⁷SO₂—;
R^{4a} is independently H, cyano, nitro, halo, —C₁₋₆ alkyl, —C₁₋₆ haloalkyl, —C₁₋₆ alkoxy, —C₁₋₆ haloalkoxy, —C₁₋₆ hydroxyalkyl, C₁₋₆ acyl-, pyrazole, 1,2,3-triazole, tetrazole, —C(═O)NR⁸R⁹, —NR⁸R⁹, C₁₋₆ acyl-N(R¹⁰)—, (C₁₋₃ alkyl)SO₂NH—, (C₁₋₆ alkyl)SO₂—, or R⁷SO₂—;
R⁷ is OH, NR⁸R⁹, O(CH₂)_{q}NR⁸R⁹, C₁₋₆ alkoxy, or C₂₋₆ hydroxyalkoxy;
R⁸ and R⁹ are independently H, —CD₃, C₁₋₆ alkyl, C₃₋₆ alkenyl, C₃₋₆ alkynyl, C₃₋₈ cycloalkyl, C₃₋₈ cycloalkenyl, C₁-C₆ acyl, 4-12 membered monocyclic or bicyclic heterocyclyl, 4-12 membered monocyclic or bicyclic heterocyclyl-C₁-C₆ alkyl-, C₆-C₁₂ aryl, 5-12 membered heteroaryl; and R⁸ and R⁹ may be further independently substituted with up to three substituents chosen from hydroxyl, C₁₋₆ alkoxy, C₁₋₆ hydroxyalkylC₂₋₆ hydroxyalkoxy, oxo, thiono, cyano or halo; or
alternatively, R⁸ and R⁹, taken together with the N atom to which they are both attached, form a heterocyclic ring of 4-7 members, containing up to one other heteroatom chosen from O, S, or NR¹¹, or a heterobicyclic ring of 7-12 members which may be fused, bridged or spiro, and contain up to two other heteroatoms chosen from O, S(O)ₓ, or NR¹¹, and these heterocyclic rings are optionally substituted with up to three substituents chosen from hydroxyl, C₁₋₆ alkoxy, C₁₋₆ hydroxyalkyl, C₁₋₆ alkoxy-C₁₋₆ alkyl, C₁₋₆ alkoxy-C₁₋₆ alkoxy, C₂₋₆ hydroxyalkoxy, oxo, thiono, cyano or halo;
each R¹⁰ is independently H, —CD₃, C₁₋₆ alkyl, C₃₋₆ cycloalkyl, C₂₋₆ hydroxyalkyl, C₁₋₆ alkoxy-C₁₋₆ alkyl or C₂₋₆ alkyl-NR⁸R⁹; or
alternatively, two R¹⁰ on the same N atom to which they are both attached, form a heterocyclic ring of 5-6 members, containing up to one other heteroatom selected from O, S, or NR¹¹; and
each R¹¹ is independently hydrogen or C₁-C₆ alkyl, which is optionally substituted with up to three substituents selected from hydroxyl, oxo, thiono, cyano and halo;
p=0, 1, 2, 3, or 4;
q=2, 3, or 4; and
x=0, 1, or 2.

In one embodiment, the compounds of formula (P) comprise:
Z is CH or N;
R¹ is hydrogen, methyl, fluoro, chloro, bromo, —CF₃, or cyano;
R³ is N(R¹⁰)C₂₋₆ alkyl-NR¹⁰R¹⁰;
each R⁴ is independently H, cyano, halo, —C₁₋₆ alkyl, —C₁₋₆ haloalkyl;
R^{4a} is independently H, cyano, nitro, halo, —C₁₋₆ alkyl, —C₁₋₆ haloalkyl, —C₁₋₆ alkoxy, —C₁₋₆ haloalkoxy, —C(═O)NR⁸R⁹, or —NR⁸R⁹;
R⁸ and R⁹ are independently H, —CD₃, —CH₃, —CH₂CH₃, or —CH(CH₃)₂; and
each R¹⁰ is independently H, —CD₃, —CH₃, —CH₂CH₃, or —CH(CH₃)₂.

In some embodiments, the compound of formula (P) is:

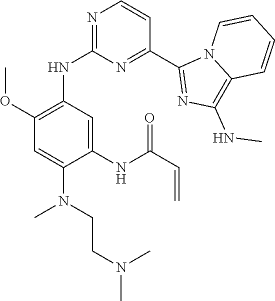 [SMILES: C=CC(=O)Nc1cc(Nc2nccc(-c3nc(NC)c4ccccn34)n2)c(OC)cc1N(C)CCN(C)C]

or a stereoisomer or a pharmaceutically acceptable salt, solvate, ester, tautomer, or prodrug thereof.

In one embodiment, the present disclosure relates to a compound having the structure:

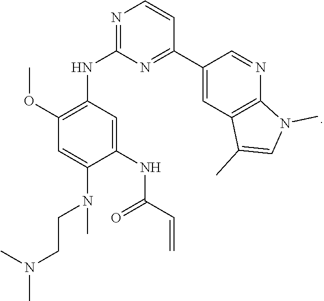

In one embodiment, the present disclosure relates to pharmaceutical compositions comprising any one of the compounds disclosed herein, or a pharmaceutically acceptable salt, solvate, ester, or prodrug thereof, and a pharmaceutically acceptable carrier. In one embodiment, the present disclosure relates to pharmaceutical compositions comprising any one of the compounds of formulae (I), (A), (B), (C), (C-I), (D), (D-I), (E), (E-I), (F), (G), (H), (H-I), (J), (K), (L), (M), (N), (O), and/or (P) as disclosed herein, or a pharmaceutically acceptable salt, solvate, N-oxide, ester, or prodrug thereof, and a pharmaceutically acceptable carrier.

In some embodiments, the present disclosure relates to methods for treating cancer in a patient in need thereof, comprising administering to the patient a therapeutically effective amount of any one of the compounds disclosed herein, or a pharmaceutically acceptable salt, solvate, ester, or prodrug thereof. In one embodiment, the cancer is selected from lung cancer, colorectal cancer, pancreatic cancer, head and neck cancers, breast cancer, ovarian cancer, uterine cancer, liver cancer, and stomach cancer. In other embodiments, the cancer is non-small cell lung cancer (NSCLC).

In some embodiments, the present disclosure relates to methods for treating cancer in a patient in need thereof, comprising administering to the patient a therapeutically effective amount of any one of the compounds disclosed herein, or a pharmaceutically acceptable salt, solvate, ester, or prodrug thereof. In one embodiment, the cancer results from a mutation in the exon 20 domain of EGFR. In some embodiments, the mutation in the exon 20 domain of EGFR is selected from NPG, ASV, or T790M. In a further embodiment, the mutation in the exon 20 domain of EGFR is T790M concurrent with an exon 19 insertion mutation or an exon 21 point mutation.

In one embodiment of any one of the methods disclosed herein, the patient is resistant to a kinase inhibitor other that a compound of any one of the compounds disclosed herein, or a pharmaceutically acceptable salt, solvate, ester, or prodrug thereof. In one embodiment, the kinase inhibitor is an EGFR inhibitor.

In one embodiment, the present disclosure relates to methods for inhibiting EGFR, or a mutation thereof, in a patient in need thereof, comprising administering to the patient a therapeutically effective amount of a compound according to any one of the compounds disclosed herein, or a pharmaceutically acceptable salt, solvate, ester, or prodrug thereof. In one embodiment, the mutation is in the exon 20 domain of EGFR.

In one embodiment, the present disclosure relates to a pharmaceutical composition comprising a compound of the invention or a pharmaceutically acceptable salt, solvate, ester, or prodrug thereof, and a pharmaceutically acceptable carrier.

In one embodiment, the present disclosure relates to a method for treating cancer in a patient in need thereof, comprising administering to the patient a therapeutically effective amount of a compound of the invention or a pharmaceutically acceptable salt, solvate, ester, or prodrug thereof.

In one embodiment, the method disclosed herein is useful for treating cancer selected from lung cancer, colorectal cancer, pancreatic cancer, head and neck cancers, breast cancer, ovarian cancer, uterine cancer, liver cancer, and stomach cancer. In another embodiment, the cancer is non-small cell lung cancer (NSCLC).

In one embodiment, the method disclosed herein relates to treatment of cancer, wherein the cancer results from a mutation in the exon 20 domain of EGFR. In some embodiments, the mutation in the exon 20 domain of EGFR is selected from NPG, ASV, or T790M. In one embodiment, the mutation in the exon 20 domain of EGFR is T790M concurrent with an exon 19 insertion mutation or an exon 21 point mutation.

In one embodiment, the method disclosed herein relates to treatment of cancer, wherein the patient is resistant to a kinase inhibitor other that a compound of the invention or a pharmaceutically acceptable salt, solvate, ester, or prodrug thereof. In another embodiment, the kinase inhibitor is an EGFR inhibitor.

The present disclosure also relates to a method for inhibiting EGFR, or a mutation thereof, in a patient in need thereof, comprising administering to the patient a therapeutically effective amount of a compound of the invention or a pharmaceutically acceptable salt, solvate, ester, or prodrug thereof. In one embodiment, the mutation is in the exon 20 domain of EGFR.

In one embodiment, the compound useful in any one of the methods as disclosed herein is a compound of formulae (I), (A), (B), (C), (C-I), (D), (D-I), (E), (E-I), (F), (G), (H), (H-I), (J), (K), (L), (M), (N), (O), and/or (P), as disclosed herein, or a pharmaceutically acceptable salt, solvate, N-oxide, ester, or prodrug thereof.

### DETAILED DESCRIPTION

### Definitions

The term “alkyl” refers to a saturated, monovalent aliphatic hydrocarbon radical including straight chain and branched chain groups having the specified number of carbon atoms. The term “C₁₋₆ alkyl” or “C₁-C₆ alkyl” refers to a branched or straight chained alkyl radical containing from 1 to 6 carbon atoms, such as methyl, ethyl, n-propyl, isopropyl, n-butyl, isobutyl, sec butyl, t-butyl, pentyl, hexyl, and the like. Similarly, the term “C₁₋₄ alkyl” or “C₁-C₄ alkyl” refers to a branched or straight chained alkyl radical containing from 1 to 4 carbon atoms, such as methyl, ethyl, n-propyl, isopropyl, n-butyl, isobutyl, sec-butyl, t-butyl, and the like.

As used herein, the term “halogen” or “halo” refers to fluoro, chloro, bromo, or iodo (F, Cl, Br, I), and in some instances, substituted alkyl groups may be specifically named with reference to the substituent group. For example, “haloalkyl” refers to an alkyl group having the specified number of carbon atoms that is substituted by one or more halo substituents, and typically contain 1-6 carbon atoms and 1, 2 or 3 halo atoms (i.e., “C₁-C₆ haloalkyl”). Thus, a C₁-C₆ haloalkyl group includes trifluoromethyl (—CF₃) and difluoromethyl (—CF₂H).

Similarly, “hydroxyalkyl” refers to an alkyl group having the specified number of carbon atoms that is substituted by one or more hydroxy substituents, and typically contain 1-6 carbon atoms and 1, 2 or 3 hydroxy (i.e., “C₁-C₆ hydroxyalkyl”). Thus, C₁-C₆hydroxyalkyl includes hydroxymethyl (—CH₂OH) and 2-hydroxyethyl (—CH₂CH₂OH).

The term “C₁₋₆ alkoxy”, “C₁-C₆ alkoxy” or “OC₁₋₆ alkyl” refers to a straight or branched alkoxy group containing from 1 to 6 carbon atoms, such as methoxy, ethoxy, n-propoxy, isopropoxy, n-butoxy, isobutoxy, sec-butoxy, t-butoxy, pentoxy, hexoxy, and the like. The term “C₁₋₄ alkoxy”, “C₁-C₄ alkoxy”, “OC₁₋₄ alkyl” refers to a straight or branched alkoxy group containing from 1 to 4 carbon atoms, such as methoxy, ethoxy, n-propoxy, isopropoxy, n-butoxy, isobutoxy, sec-butoxy, t-butoxy, and the like.

The term “C₃₋₆ cycloalkoxy”, “C₃-C₆ cycloalkoxy”, or “OC₃₋₆ cycloalkyl” refers to a cyclic alkoxy radical containing from 3 to 6 carbon atoms such as cyclopropoxy, cyclobutoxy, cyclopentoxy, and the like.

“Alkoxyalkyl” refers to an alkyl group having the specified number of carbon atoms that is substituted by one or more alkoxy substituents. Alkoxyalkyl groups typically contain 1-6 carbon atoms in the alkyl portion and are substituted by 1, 2 or 3 C₁-C₄ alkyoxy substituents. Such groups are sometimes described herein as C₁-C₄ alkyoxy-C₁-C₆ alkyl. “Aminoalkyl” refers to alkyl group having the specified number of carbon atoms that is substituted by one or more substituted or unsubstituted amino groups, as such groups are further defined herein.

Aminoalkyl groups typically contain 1-6 carbon atoms in the alkyl portion and are substituted by 1, 2 or 3 amino substituents. Thus, a C₁-C₆ aminoalkyl group includes, for example, aminomethyl (—CH₂NH₂), N,N-dimethylamino-ethyl (—CH₂CH₂N(CH₃)₂), 3-(N-cyclopropylamino)propyl (—CH₂CH₂CH₂NH—^{C}Pr) and N-pyrrolidinylethyl (—CH₂CH₂N-pyrrolidinyl).

“Alkenyl” refers to an alkyl group, as defined herein, consisting of at least two carbon atoms and at least one carbon-carbon double bond. Typically, alkenyl groups have 2 to 20 carbon atoms (“C₂-C₂₀ alkenyl”), preferably 2 to 12 carbon atoms (“C₂-C₁₂ alkenyl”), more preferably 2 to 8 carbon atoms (“C₂-C₈ alkenyl”), or 2 to 6 carbon atoms (“C₂-C₆ alkenyl”), or 2 to 4 carbon atoms (“C₂₋₄ alkenyl”). Representative examples include ethenyl, 1-propenyl, 2-propenyl, 1-, 2-, or 3-butenyl, and the like. A “C₂-C₆ alkenyl” denotes a straight-chain or branched group containing 2 to 6 carbon atoms and at least one double bond between two sp² hybridized carbon atoms. This also applies if they carry substituents or occur as substituents of other radicals, for example in O—(C₂-C₆) alkenyl radicals. Examples of suitable C₂-C₆ alkenyl radicals are n-propenyl, isopropenyl, n-butenyl, iso-butenyl, n-pentenyl, sec-pentenyl, n-hexenyl, sec-hexenyl, and the like. Alkenyl groups may be unsubstituted or substituted by the same groups that are described herein as suitable for alkyl.

“Alkynyl” refers to an alkyl group, as defined herein, consisting of at least two carbon atoms and at least one carbon-carbon triple bond. Alkynyl groups have 2 to 20 carbon atoms (“C₂-C₂₀ alkynyl”), preferably 2 to 12 carbon atoms (“C₂-C₁₂ alkynyl”), more preferably 2 to 8 carbon atoms (“C₂-C₈ alkynyl”), or 2 to 6 carbon atoms (“C₂-C₆ alkynyl”), or 2 to 4 carbon atoms (“C₂-C₄ alkynyl”). Representative examples include, but are not limited to, ethynyl, 1-propynyl, 3-propynyl, 1-, 3-, or 4-butynyl, and the like. Alkynyl groups may be unsubstituted or substituted by the same groups that are described herein as suitable for alkyl. A “C₂-C₆ alkynyl” denotes a straight-chain or branched group containing 2 to 6 carbon atoms and at least one triple bond between two sp hybridized carbon atoms. This also applies if they carry substituents or occur as substituents of other radicals, for example in O—(C₂-C₆)alkynyl radicals. Examples of suitable C₂-C₆ alkynyl radicals are propynyl, butynyl, pentynyl, hexynyl, and the like.

“Alkylene” as used herein refers to a divalent hydrocarbyl group having the specified number of carbon atoms which can link two other groups together. Sometimes it refers to —(CH₂)n- where n is 1-8, and preferably n is 1-4. Similarly as used herein, m, q, and p can be each 1-8 or 0, which denotes absence of the methylene unit. Where specified, an alkylene can also be substituted by other groups and may include one or more degrees of unsaturation (i.e., an alkenylene or alkynylene moiety) or rings. The open valences of an alkylene need not be at opposite ends of the chain. Thus —CH(Me)— and —C(Me)₂— are also included within the scope of the term ‘alkylenes’, as are cyclic groups such as cyclopropan-1,1-diyl and unsaturated groups such as ethylene (—CH═CH—) or propylene (—CH₂CH═CH—). Where an alkylene group is described as optionally substituted, the substituents include those typically present on alkyl groups as described herein.

“Heteroalkylene” refers to an alkylene group as described above, wherein one or more non-contiguous carbon atoms of the alkylene chain are replaced by —N—, —O—, —P— or —S—, in manifestations such as —N(R)—, —P(═O)(R). —S(O)ₓ— or —S(═O)(═NR)—, where R is H or C₁-C₄ alkyl and x is 0-2. For example, the group —O—(CH₂)₁₋₄— is a ‘C₂-C₅’-heteroalkylene group, where one of the carbon atoms of the corresponding alkylene is replaced by O.

“Aryl” or “aromatic” refers to an all-carbon monocyclic or fused-ring polycyclic having a completely conjugated pi-electron system and possessing aromaticity. The terms “C₆-C₁₂ aryl” and “C₆₋₁₂ aryl” are included within this term and encompass aromatic ring systems of 6 to 12 carbons and containing no heteroatoms within the ring system. Examples of aryl groups are phenyl and naphthalenyl. The aryl group may be substituted or unsubstituted. Substituents on adjacent ring carbon atoms of a C₆-C₁₂ aryl may combine to form a 5- or 6-membered carbocyclic ring optionally substituted by one or more substituents, such as oxo, C₁-C₆ alkyl, hydroxyl, amino and halogen, or a 5- or 6-membered heterocyclic ring containing one, two or three ring heteroatoms selected from N, O and S(O)ₓ (where x is 0, 1 or 2) optionally substituted by one or more substituents, such as oxo, C₁-C₆ alkyl, hydroxyl, amino and halogen. Examples of aryl groups include phenyl, biphenyl, naphthyl, anthracenyl, phenanthrenyl, indanyl, indenyl, and tetrahydronaphthyl. The aryl group may be unsubstituted or substituted as further described herein.

“Heteroaryl” or “heteroaromatic” refers to monocyclic or fused bicyclic or polycyclic ring systems having the well-known characteristics of aromaticity that contain the specified number of ring atoms and include at least one heteroatom selected from N, O, and S as a ring member in an aromatic ring. The inclusion of a heteroatom permits aromaticity in 5-membered rings as well as 6-membered rings. Typically, heteroaryl groups contain 5 to 20 ring atoms (“5-20 membered heteroaryl”), preferably 5 to 14 ring atoms (“5-14 membered heteroaryl”), and more preferably 5 to 12 ring atoms (“5-12 membered heteroaryl”) or 5 to 6 ring atoms (“5-6 membered heteroaryl”). Heteroaryl rings are attached to the base molecule via a ring atom of the heteroaromatic ring, such that aromaticity is maintained. Thus, 6-membered heteroaryl rings may be attached to the base molecule via a ring C atom, while 5-membered heteroaryl rings may be attached to the base molecule via a ring C or N atom. The heteroaryl group may be unsubstituted or substituted as further described herein. As used herein, “5-6 membered heteroaryl” refers to a monocyclic group of 5 or 6 ring atoms containing one, two or three ring heteroatoms selected from N, O, and S, but including tetrazolyl with 4 nitrogens, the remaining ring atoms being C, and, in addition, having a completely conjugated pi-electron system. Substituents on adjacent ring atoms of a 5- or 6-membered heteroaryl may combine to form a fused 5- or 6-membered carbocyclic ring optionally substituted by one or more substituents, such as oxo, C₁-C₆ alkyl, hydroxyl, amino and halogen, or a fused 5- or 6-membered heterocyclic ring containing one, two or three ring heteroatoms selected from N, O, and S(O)x (where x is 0, 1 or 2) optionally substituted by one or more substituents, such as oxo, C₁-C₆ alkyl, hydroxyl, amino and halogen. If said fused ring is itself aromatic, it is referred to as a fused (bicyclic) heteroaromatic species, regardless of whether the second ring contains heteroatoms. A pharmaceutically acceptable heteroaryl is one that is sufficiently stable to be attached to a compound of the invention, formulated into a pharmaceutical composition and subsequently administered to a patient in need thereof.

Examples of 5-membered heteroaryl rings containing 1, 2 or 3 heteroatoms independently selected from O, N, and S, include pyrrolyl, thienyl, furanyl, pyrazolyl, imidazolyl, oxazolyl, isoxazolyl, thiazolyl, isothiazolyl, triazolyl, tetrazolyl, oxadiazolyl and thiadiazolyl. Preferred 6-membered heteroaryl rings contain 1 or 2 nitrogen atoms. Examples of 6-membered heteroaryl are pyridyl, pyridazinyl, pyrimidinyl and pyrazinyl. Examples of fused heteroaryl rings include benzofuran, benzothiophene, indole, benzimidazole, indazole, quinolone, isoquinoline, purine, pyrrolopyrimidine, napthyridine and carbazole.

An “arylene” as used herein refers to a bivalent radical derived from an aromatic hydrocarbon by removal of a hydrogen atom from each of two carbon atoms of the nucleus. In frequent embodiments, the arylene ring is a 1,2-disubstituted or a 1,3-disubstituted arylene. The aryl ring of the arylene moiety may be optionally substituted on open valence positions with groups suitable for an aryl ring, to the extent such substitution is indicated. Preferably, the arylene ring is a C₆-C₁₂ arylene ring, for example a 1,2-phenylene or 1,3-phenylene moiety.

Similarly, a “heteroarylene” as used herein refers to a bivalent radical derived from a heteroaromatic ring by removal of a hydrogen atom from each of two carbon or a carbon atom and a nitrogen atom of the nucleus. In frequent embodiments, the heteroarylene ring is a 1,2-disubstituted or a 1,3-disubstituted heteroarylene. The heteroaryl ring of the heteroarylene moiety is optionally substituted with groups suitable for an heteroaryl ring, to the extent such substitution is indicated. Preferably, the heteroarylene ring is a 5-12 membered, possibly fused, heteroarylene ring, more preferably a 5-6 membered heteroarylene ring, each of which may be optionally substituted.

The terms “heteroalicyclic”, “heterocyclyl”, or “heterocyclic” may be used interchangeably herein to refer to a non-aromatic, saturated or partially unsaturated ring system containing the specified number of ring atoms, including at least one heteroatom selected from N, O, and S as a ring member, wherein the heterocyclic ring is connected to the base molecule via a ring atom, which may be C or N. Heteroalicyclic rings may be fused to one or more other heteroalicyclic or carbocyclic rings, which fused rings may be saturated, partially unsaturated or aromatic. Preferably, heteroalicyclic rings contain 1 to 4 heteroatoms selected from N, O, and S as ring members, and more preferably 1 to 2 ring heteroatoms, provided that such heteroalicyclic rings do not contain two contiguous oxygen atoms. Heteroalicyclic groups may be unsubstituted or substituted by the same groups that are described herein as suitable for alkyl, aryl or heteroaryl.

Preferred heteroalicyclic groups include 3-12 membered heteroalicyclic groups, 5-8 membered heterocyclyl (or heteroalicyclic) groups, 4-12 membered heteroalicyclic monocycles, and 6-12 membered heteroalicyclic bicycles in accordance with the definition herein. As used herein, “3-12 membered heteroalicyclic” refers to a monocyclic or bicyclic group having 3 to 12 ring atoms, in which one, two, three or four ring atoms are heteroatoms selected from N, O, P(O), S(O)x (where x is 0, 1, 2) and S(═O)(═NR) the remaining ring atoms being C. The ring may also have one or more double bonds. However, the ring does not have a completely conjugated pi-electron system. Substituents on two ring carbon atoms may combine to form a 5- or 6-membered bridged ring that is either carbocyclic or heteroalicyclic containing one, two or three ring heteroatoms selected from N, O and S(O)x (where x is 0, 1 or 2). The heteroalicyclic group is optionally substituted by oxo, hydroxyl, amino, C₁-C₆-alkyl and the like.

In frequent embodiments, heteroalicyclic groups contain 3-12 ring members, including both carbon and non-carbon heteroatoms, and preferably 4-6 ring members. In certain preferred embodiments, substituent groups comprising 3-12 membered heteroalicyclic groups are selected from azetidinyl, pyrrolidinyl, piperidinyl, piperazinyl, morpholinyl and thiomorpholinyl rings, each of which may be optionally substituted to the extent such substitution makes chemical sense.

It is understood that no more than two N, O, P, or S atoms are ordinarily connected sequentially, except where an oxo or aza group is attached to N, P or S in a higher formal oxidation state than its basal state (eg N⁵⁺, P⁵⁺, S⁶⁺) to form groups such as, but not limited to, nitro, phosphinyl, phosphinamido, sulfoximino and sulfonyl group, or in the case of certain heteroaromatic rings, such as triazine, triazole, tetrazole, oxadiazole, thiadiazole, and the like.

“Cycloalkyl” refers to a non-aromatic, saturated or partially unsaturated carbocyclic ring system containing the specified number of carbon atoms, which may be a monocyclic, bridged, fused, or spiral bicyclic or polycyclic ring system that is connected to the base molecule through a carbon atom of the cycloalkyl ring. Typically, the cycloalkyl groups of the invention contain 3 to 12 carbon atoms (“C3-C12 cycloalkyl”), preferably 3 to 8 carbon atoms (“C3-C8 cycloalkyl”). Other cycloalkyl groups include partially unsaturated moieties from 4 to 7 carbons (“C4-C7 cycloalkenyl”). Representative examples include, e.g., cyclopropane, cyclobutane, cyclopentane, cyclopentene, cyclohexane, cyclohexene, cyclohexadiene, cycloheptane, cycloheptatriene, adamantane, and the like. Cycloalkyl groups may be unsubstituted or substituted by the same groups that are described herein as suitable for alkyl. As used herein, “C3-C6 cycloalkyl” refers to an all-carbon, monocyclic or fused-ring polycyclic group of 3 to 6 carbon atoms.

“Cycloalkylalkyl” may be used to describe a cycloalkyl ring, typically a C3-C8 cycloalkyl, which is connected to the base molecule through an alkylene linker, typically a C1-C4 alkylene. Cycloalkylalkyl groups are described by the total number of carbon atoms in the carbocyclic ring and linker, and typically contain from 4-12 carbon atoms (“C4-C12 cycloalkylalkyl”). Thus a cyclopropylmethyl group is a C4-cycloalkylalkyl group and a cyclohexylethyl is a C8-cycloalkylalkyl. Cycloalkylalkyl groups may be unsubstituted or substituted on the cycloalkyl and/or alkylene portions by the same groups that are described herein as suitable for alkyl groups.

An “aralkyl” group refers to an aryl group as described herein which is linked to the base molecule through an alkylene or similar linker. Aralkyl groups are described by the total number of carbon atoms in the ring and linker. Thus a benzyl group is a C7-aralkyl group and a phenylethyl is a C8-aralkyl. Typically, aralkyl groups contain 7-16 carbon atoms (“C7-C16 aralkyl”), wherein the aryl portion contains 6-12 carbon atoms and the alkylene portion contains 1-4 carbon atoms. Such groups may also be represented as —C1-C4 alkylene-C6-C12 aryl.

“Heteroaralkyl” refers to a heteroaryl group as described above that is attached to the base molecule through an alkylene linker, and differs from “aralkyl” in that at least one ring atom of the aromatic moiety is a heteroatom selected from N, O and S. Heteroaralkyl groups are sometimes described herein according to the total number of non-hydrogen atoms (i.e., C, N, S and O atoms) in the ring and linker combined, excluding substituent groups. Thus, for example, pyridinylmethyl may be referred to as a “C7”-heteroaralkyl. Typically, unsubstituted heteroaralkyl groups contain 6-20 non hydrogen atoms (including C, N, S and O atoms), wherein the heteroaryl portion typically contains 5-12 atoms and the alkylene portion typically contains 1-4 carbon atoms. Such groups may also be represented as —C1-C4 alkylene-5-12 membered heteroaryl.

Similarly, “arylalkoxy” and “heteroarylalkoxy” refer to aryl and heteroaryl groups, attached to the base molecule through a heteroalkylene linker (i.e., —O-alkylene-), wherein the groups are described according to the total number of non-hydrogen atoms (i.e., C, N, S and O atoms) in the ring and linker combined. Thus, —O—CH₂-phenyl and —OCH₂-pyridinyl groups would be referred to as C8-arylalkoxy and C8-heteroarylalkoxy groups, respectively.

Where an aralkyl, arylalkoxy, heteroaralkyl or heteroarylalkoxy group is described as optionally substituted, the substituents may be on either the divalent linker portion or on the aryl or heteroaryl portion of the group. The substituents optionally present on the alkylene or heteroalkylene portion are the same as those described above for alkyl or alkoxy groups generally, while the substituents optionally present on the aryl or heteroaryl portion are the same as those described above for aryl or heteroaryl groups generally.

“Hydroxy” refers to an —OH group.

“Acyl” refers to a monovalent group —C(O)alkyl wherein the alkyl portion has the specified number of carbon atoms (typically C1-C8, preferably C1-C6 or C1-C4) and may be substituted by groups suitable for alkyl. Thus, C1-C4 acyl includes a —C(O)C1-C4 alkyl substituent, e.g., —C(O)CH₃. Similarly, “acyloxy” refers to a monovalent group —OC(O)alkyl wherein the alkyl portion has the specified number of carbon atoms (typically C1-C8, preferably C1-C6 or C1-C4) and may be substituted by groups suitable for alkyl. Thus, C1-C4 acyloxy includes a —OC(O)C1-C4 alkyl substituent, e.g., —OC(O)CH₃.

The term “monocyclic or bicyclic ring system” refers to an aromatic, saturated or partially unsaturated ring system containing the specified number of ring atoms, and may optionally include one or more heteroatoms selected from N, O, and S as a ring member, wherein the heterocyclic ring is connected to the base molecule via a ring atom, which may be C or N. Included within this term are the terms “cycloalkyl”, “aryl”, “heterocyclyl”, and “heteroaryl”. Typically, the monocyclic or bicyclic ring system of the invention contain 4 to 12 members atoms (“4-12 membered monocyclic or bicyclic ring system”). Bicyclic systems may be connected via a 1,1-fusion (spiro), a 1,2-fusion (fused) or a 1,>2-fusion (bridgehead). Representative examples include cyclopentane, cyclopentene, cyclohexane, norbornyl, spiro[2.3]hexane, phenyl, biphenyl, naphthyl, anthracenyl, phenanthrenyl, pyrrolyl, thienyl, furanyl, pyrazolyl, imidazolyl, oxazolyl, isoxazolyl, thiazolyl, azetidinyl, pyrrolidinyl, piperidinyl, piperazinyl, benzothiophenyl, indolyl, and the like.

Representative examples of the central azine system are illustrated below, but the invention is not limited to these examples:

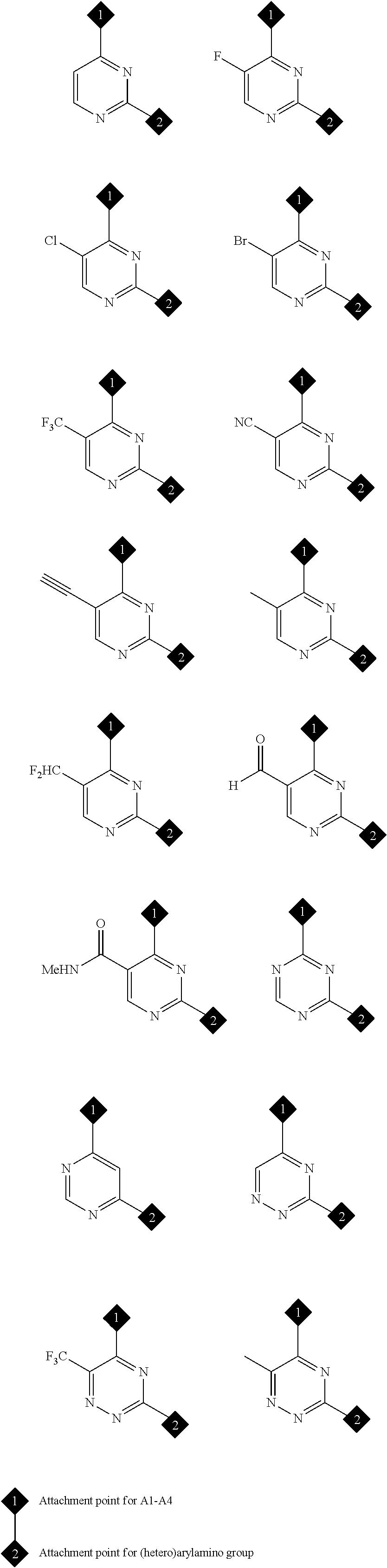

Representative examples of the 5,6-bicyclic azaaromatics which can be A¹ are illustrated below, but the invention is not limited to these examples:

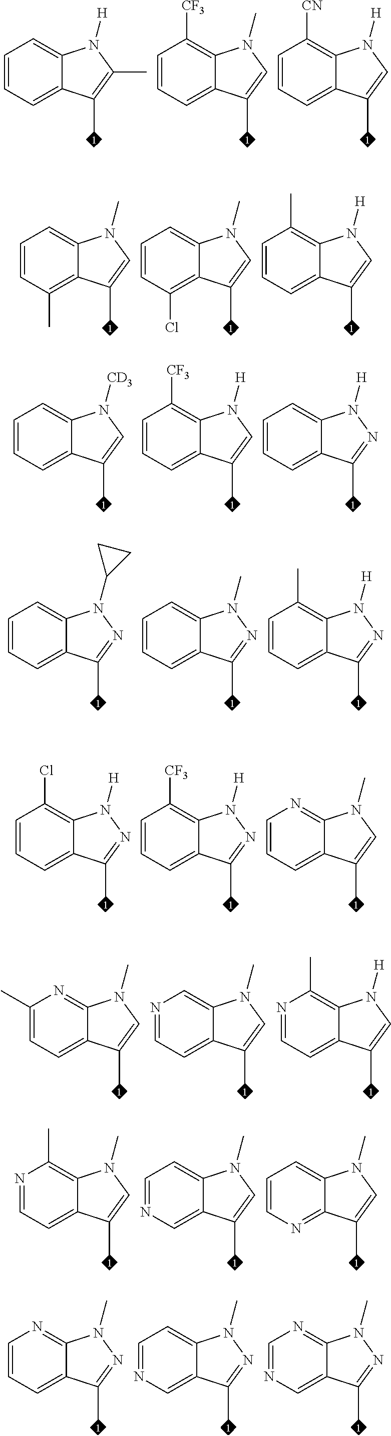

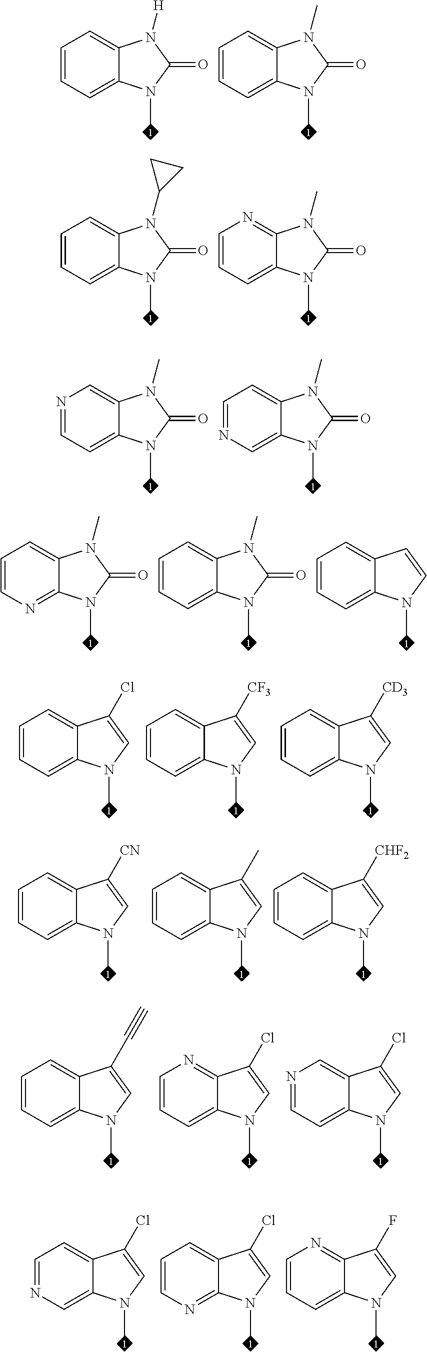

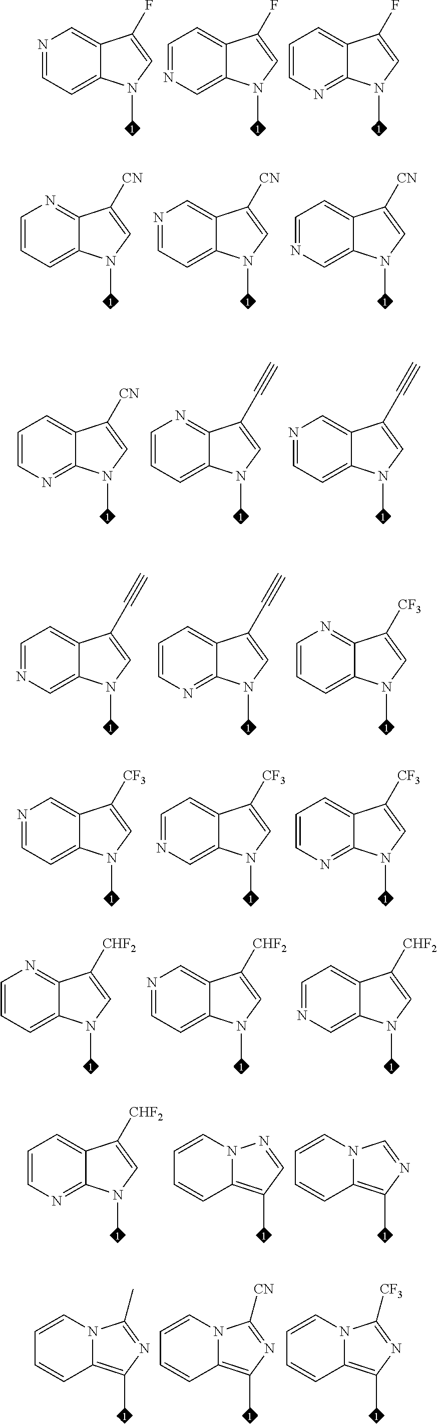

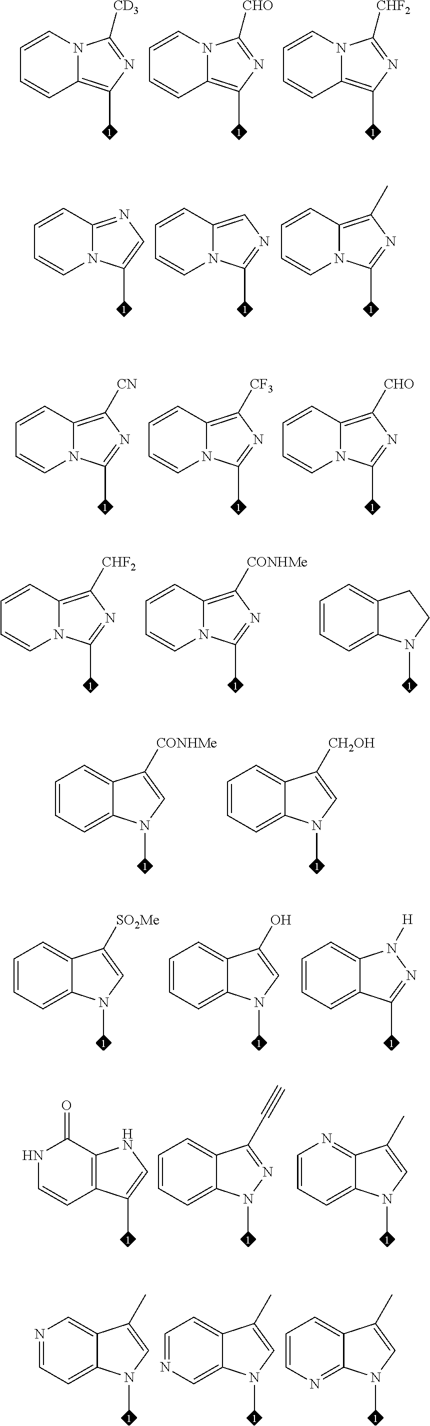

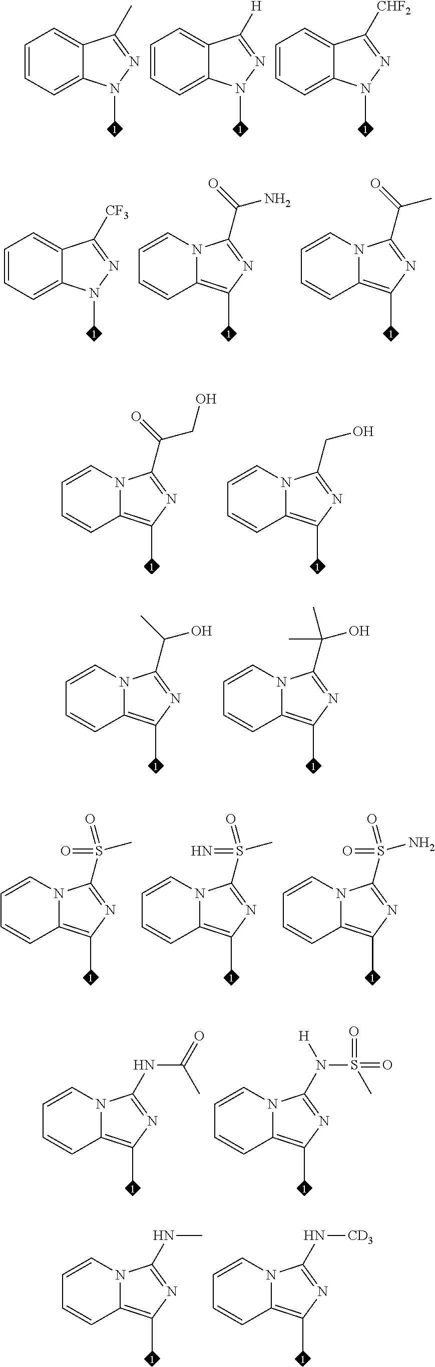

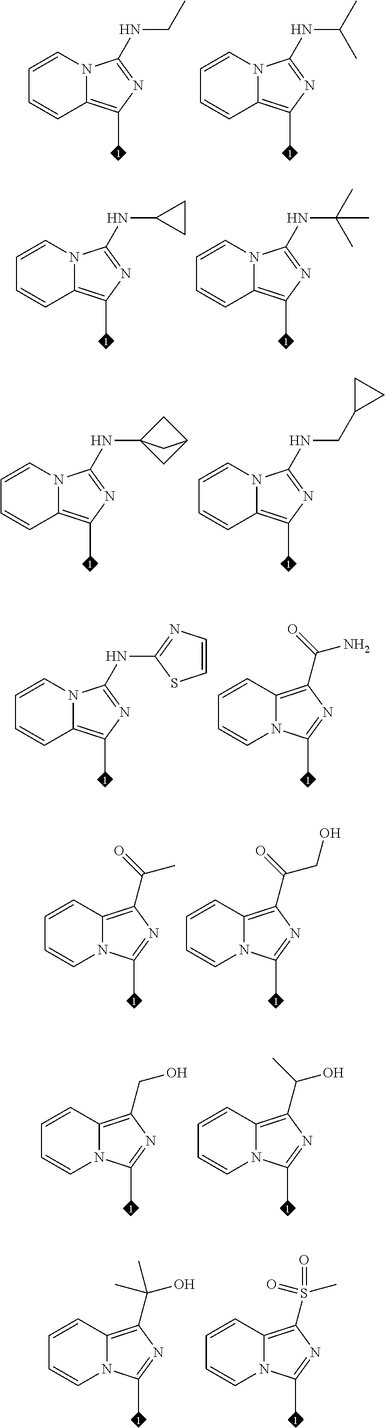

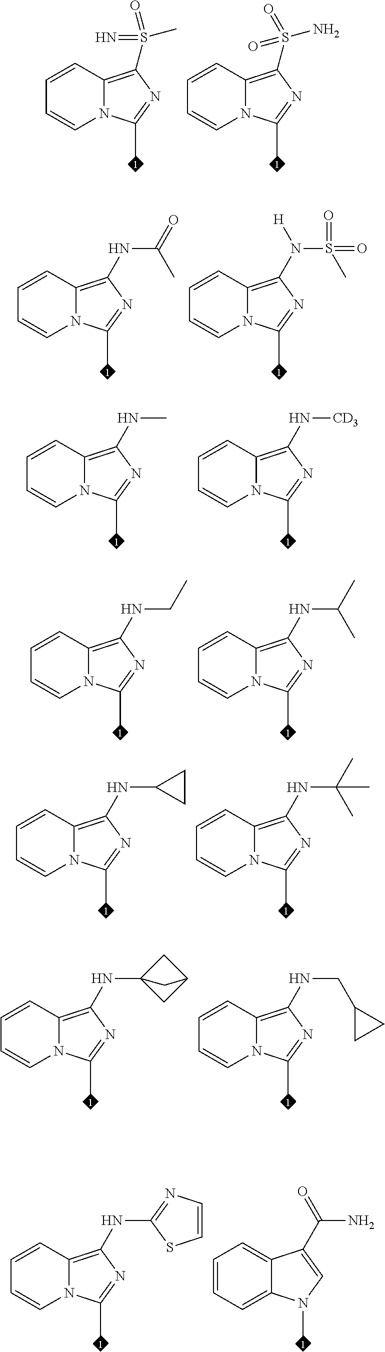

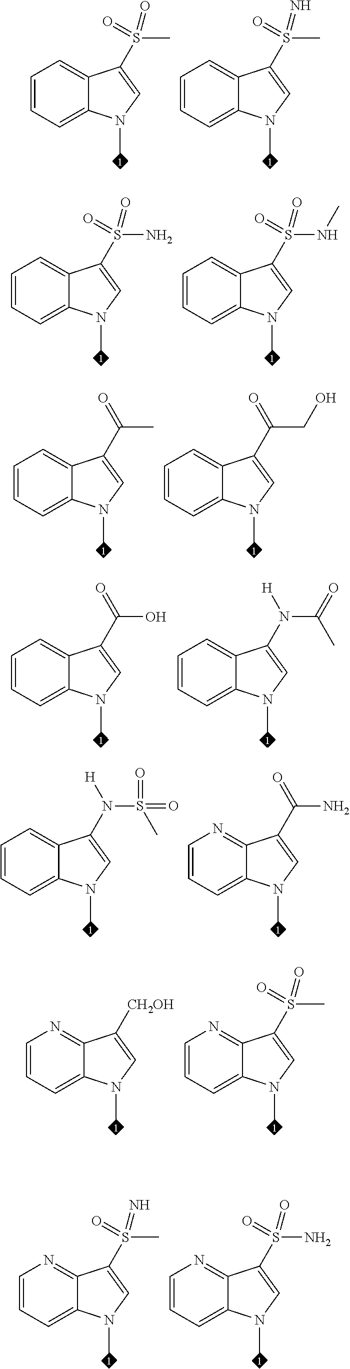

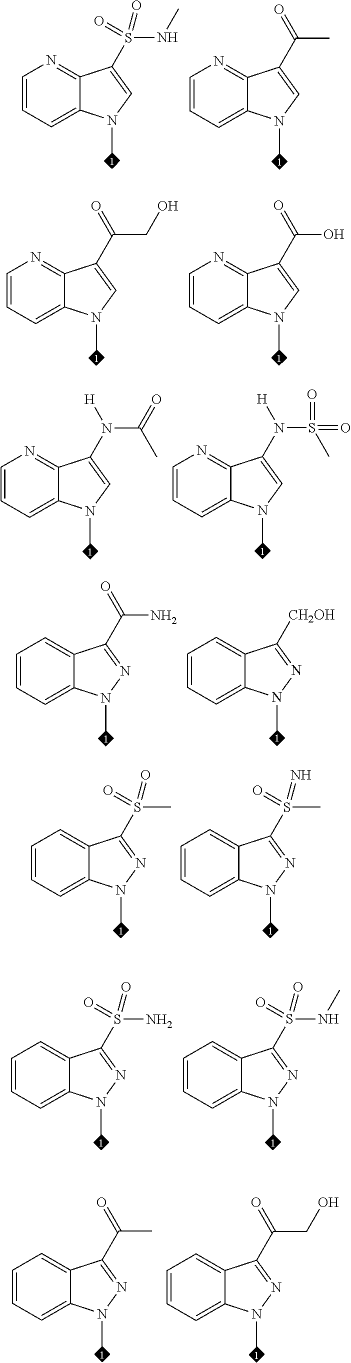

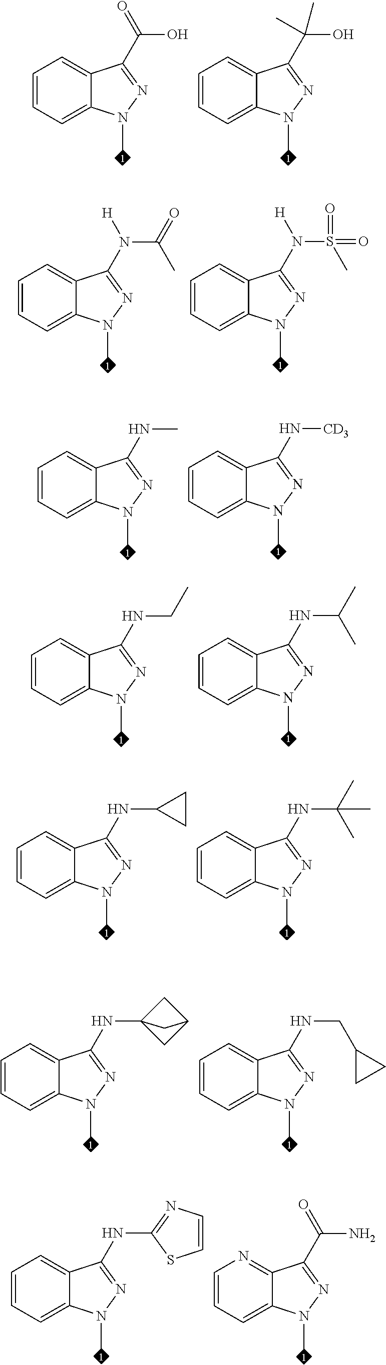

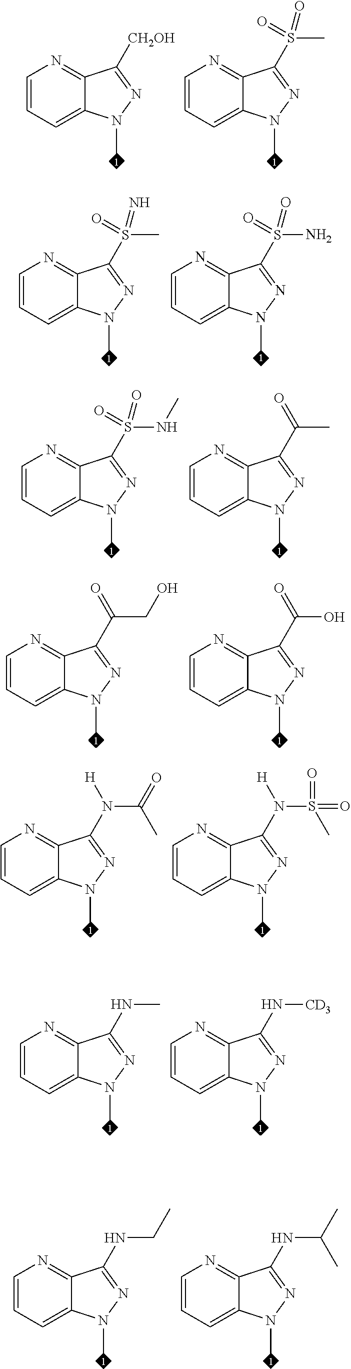

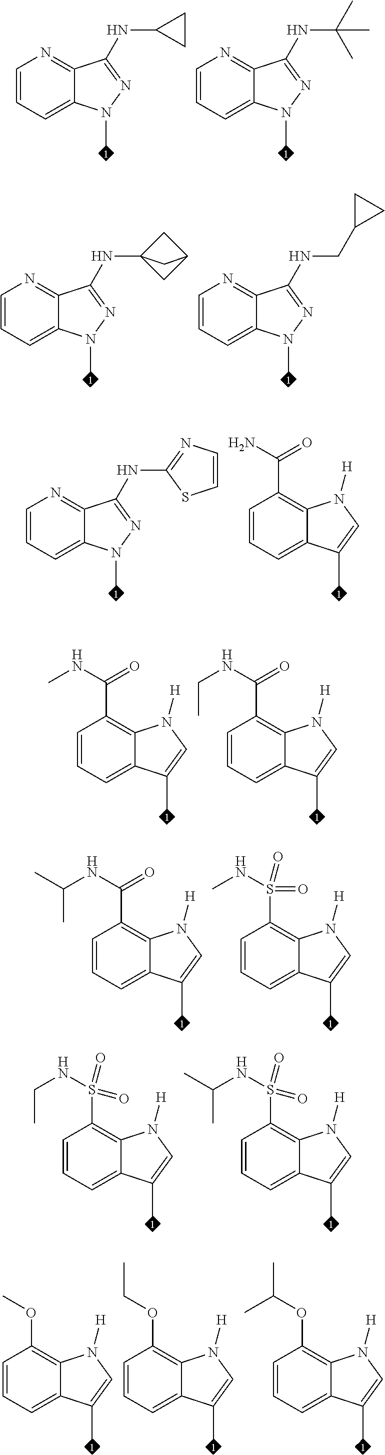

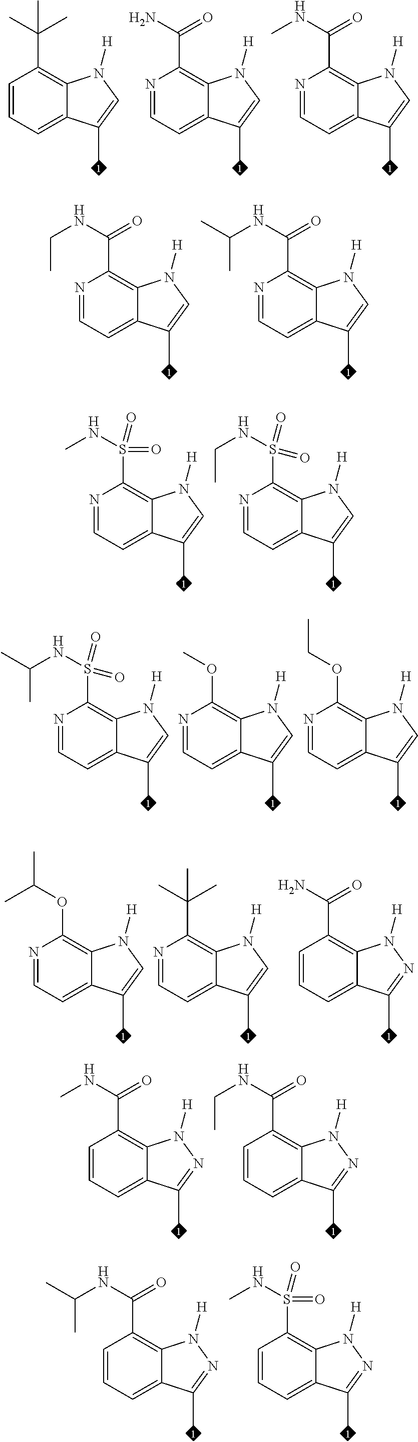

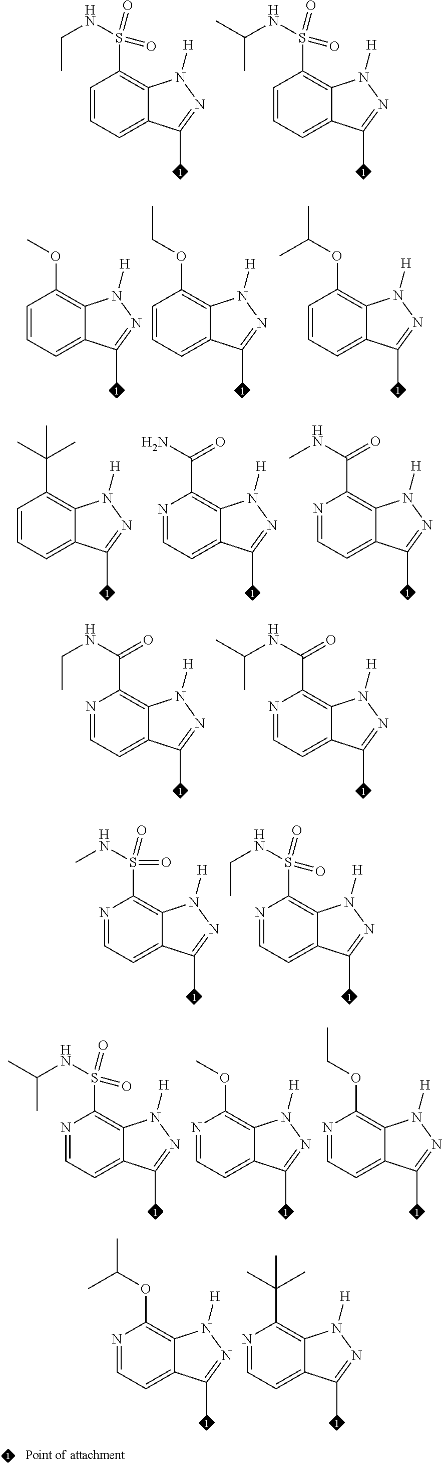

Representative examples of the partially saturated 5.× bicyclic azaaromatics which can be A¹, A², A³, or A⁶ are illustrated below, but the invention is not limited to these examples:

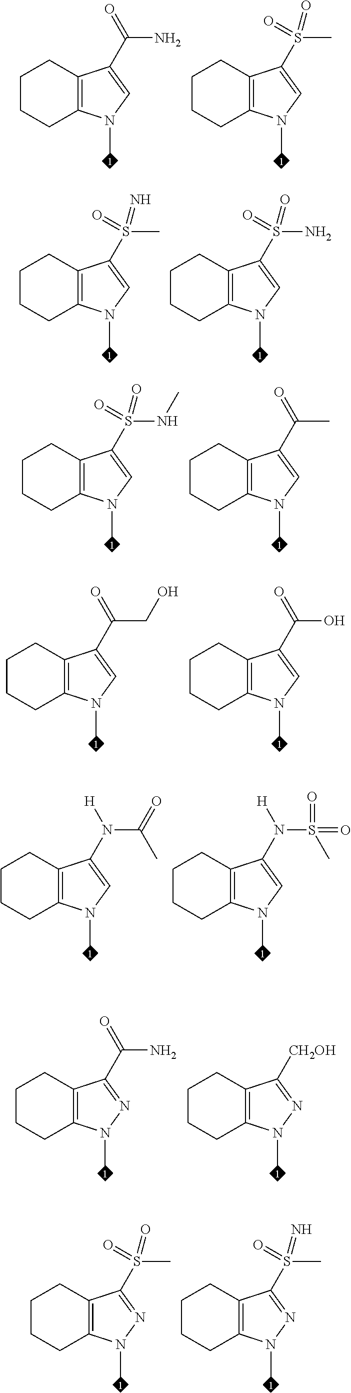

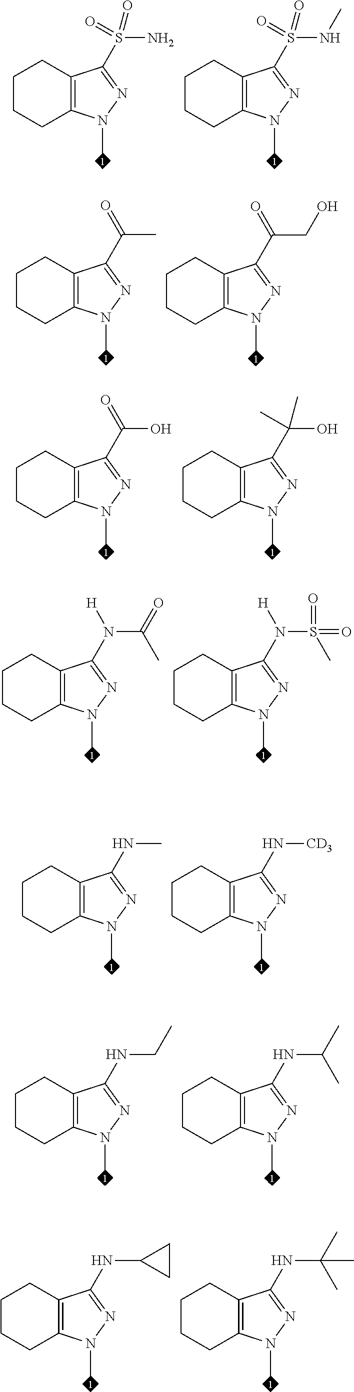

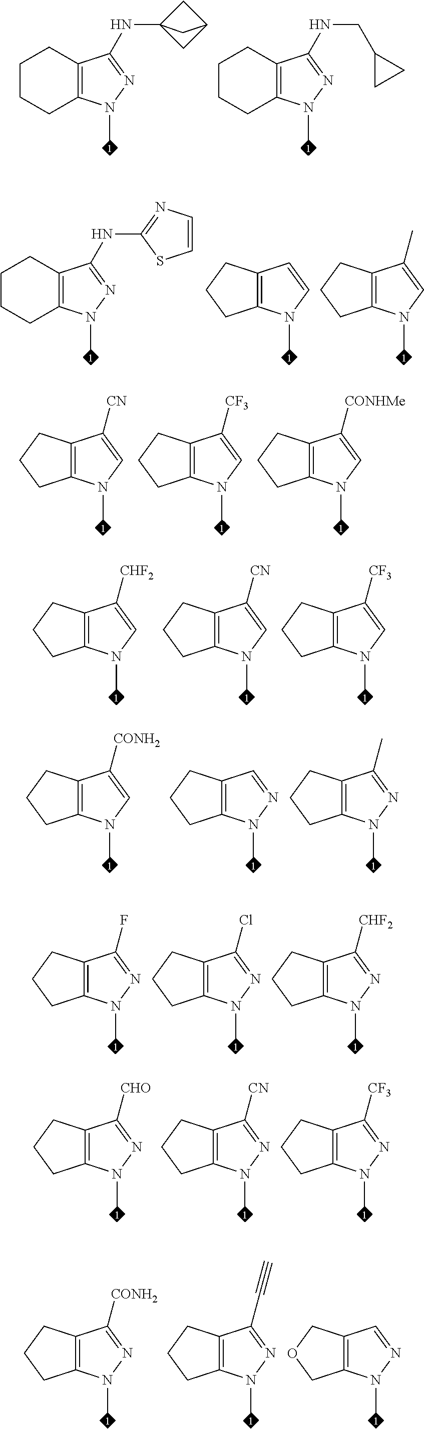

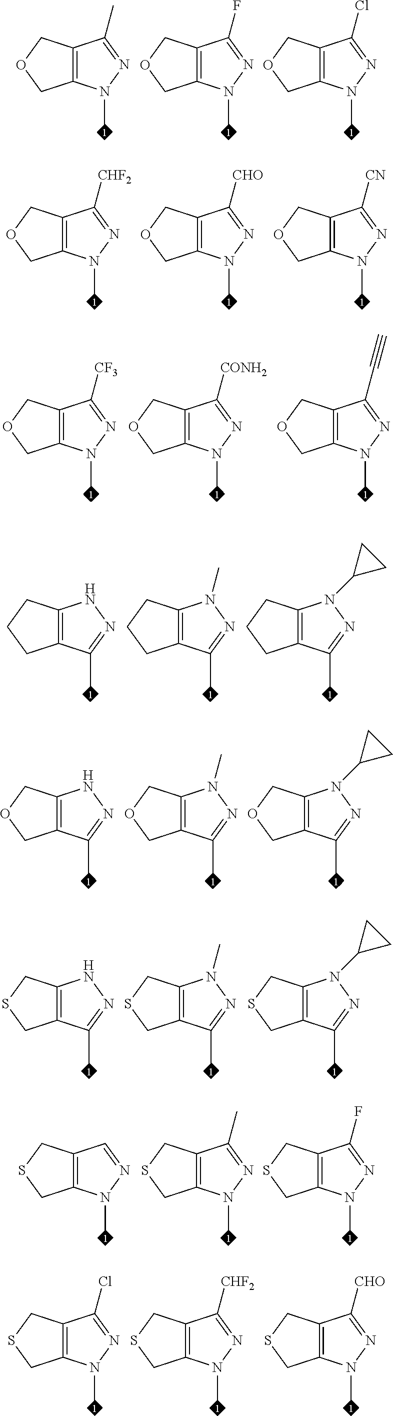

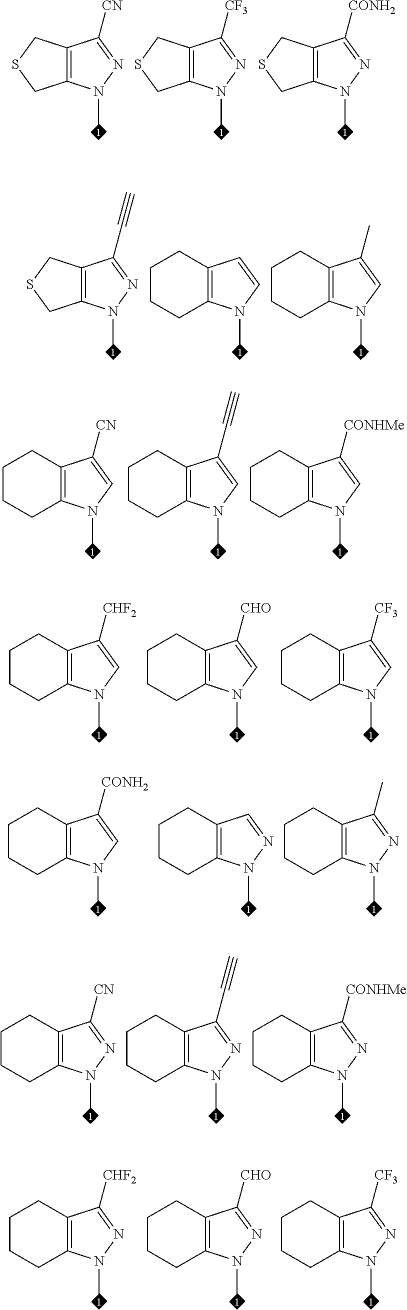

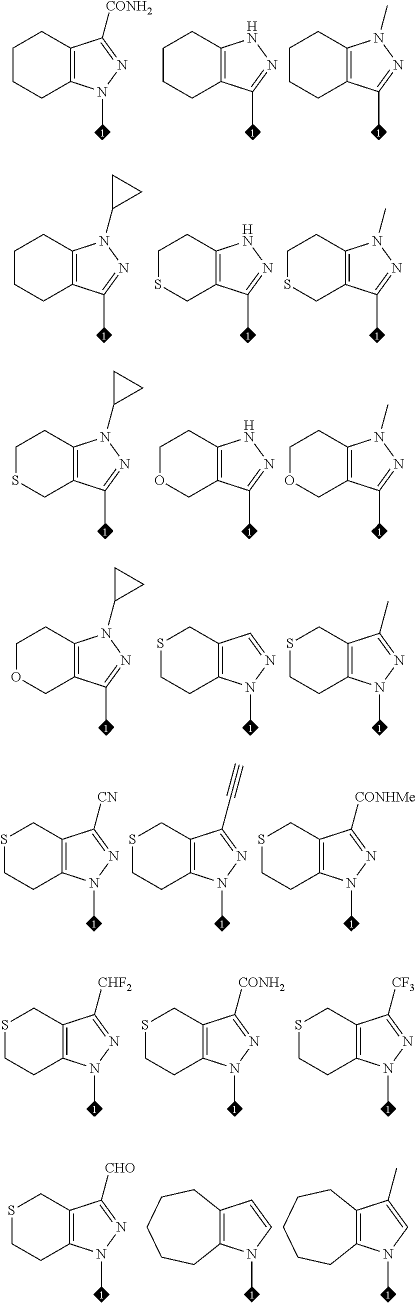

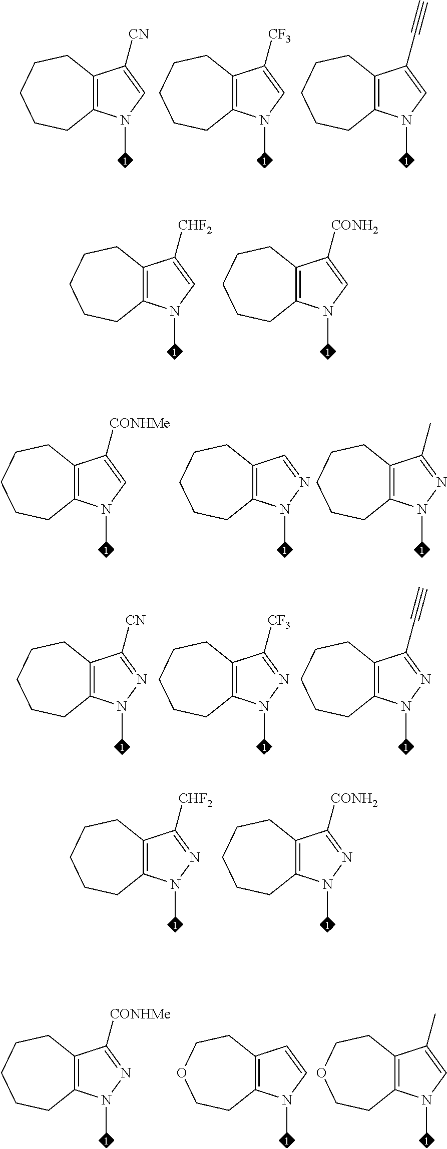

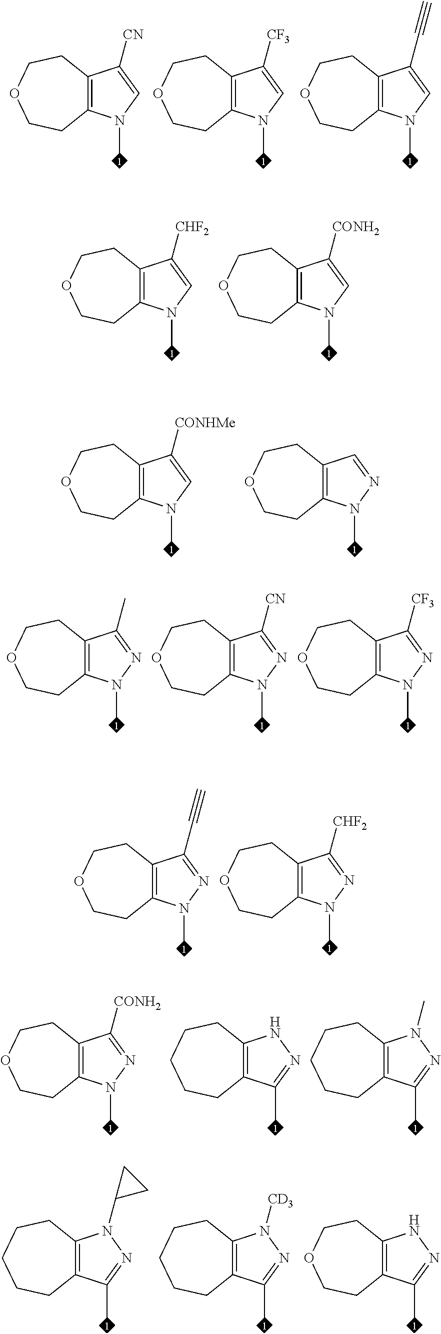

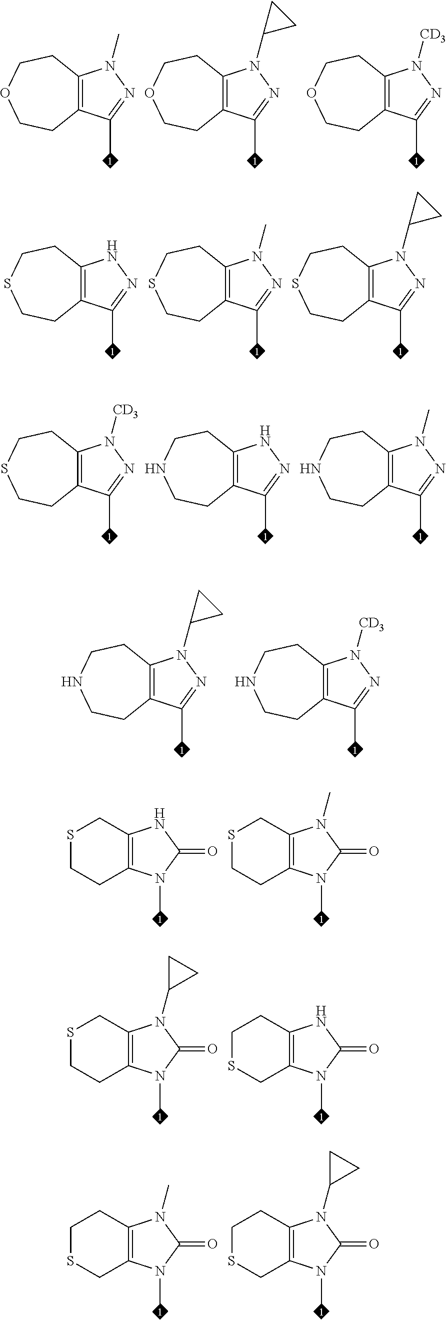

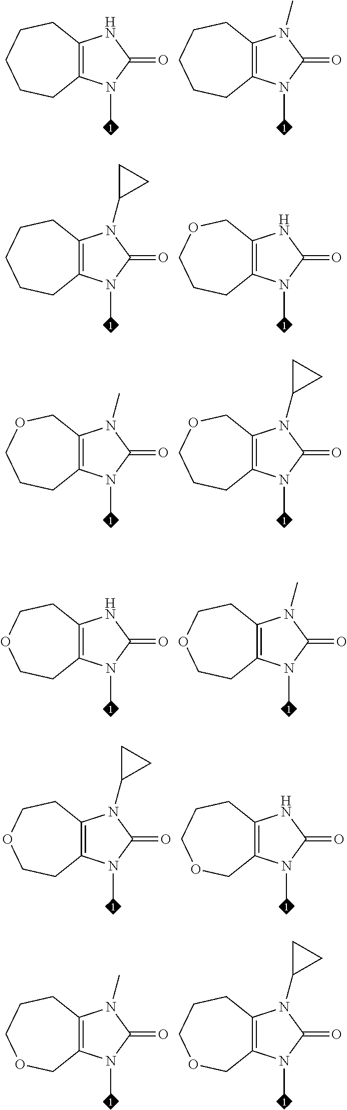

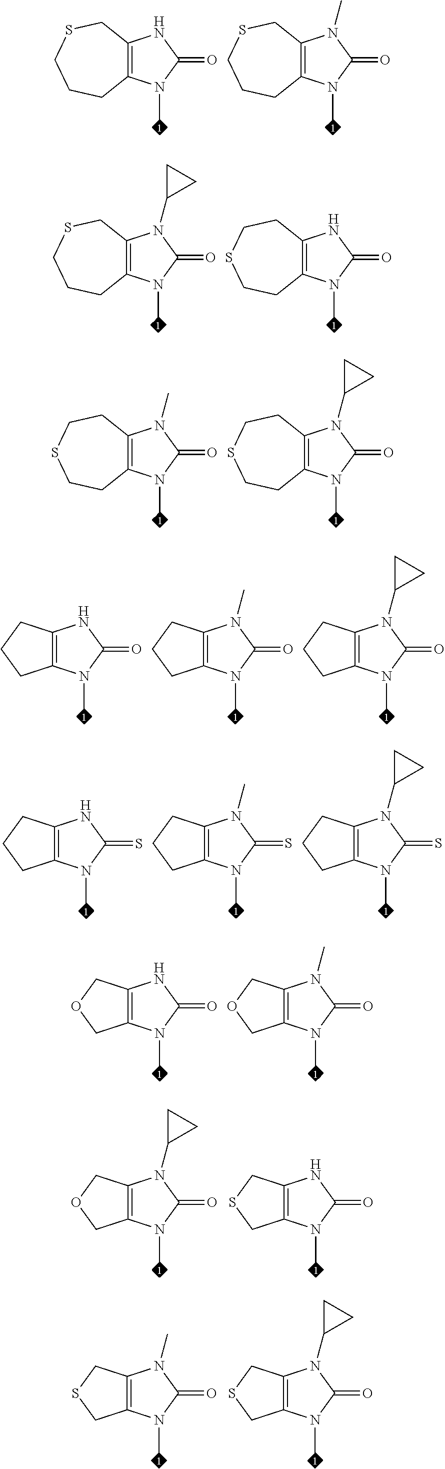

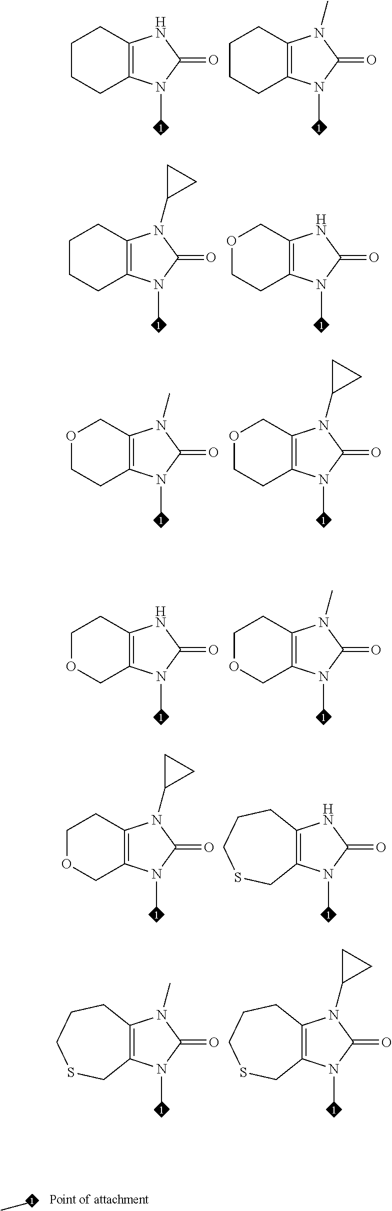

Representative examples of the 5.5 bicyclic azaaromatics which can be A³ are illustrated below, but the invention is not limited to these examples:

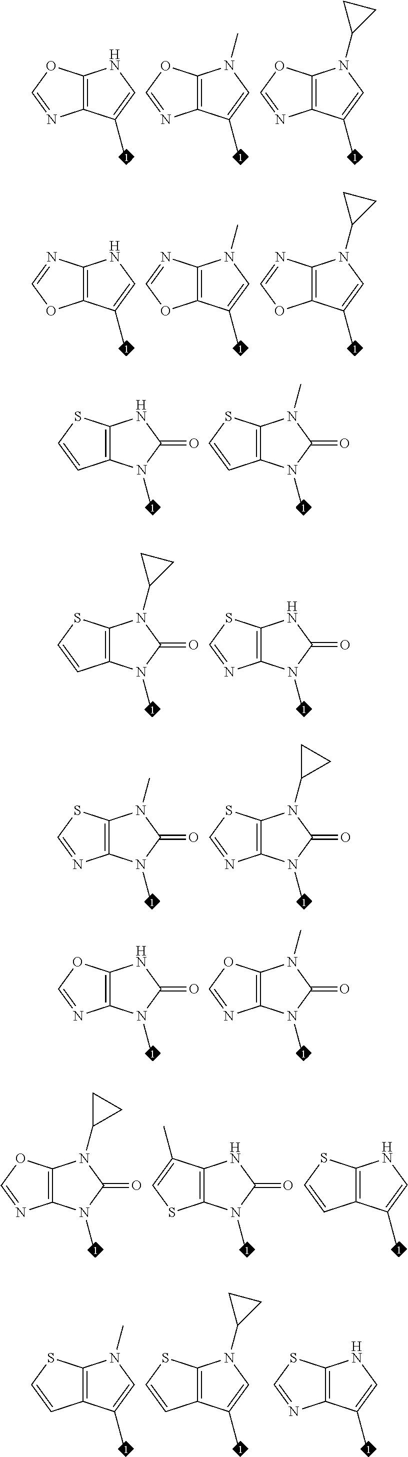

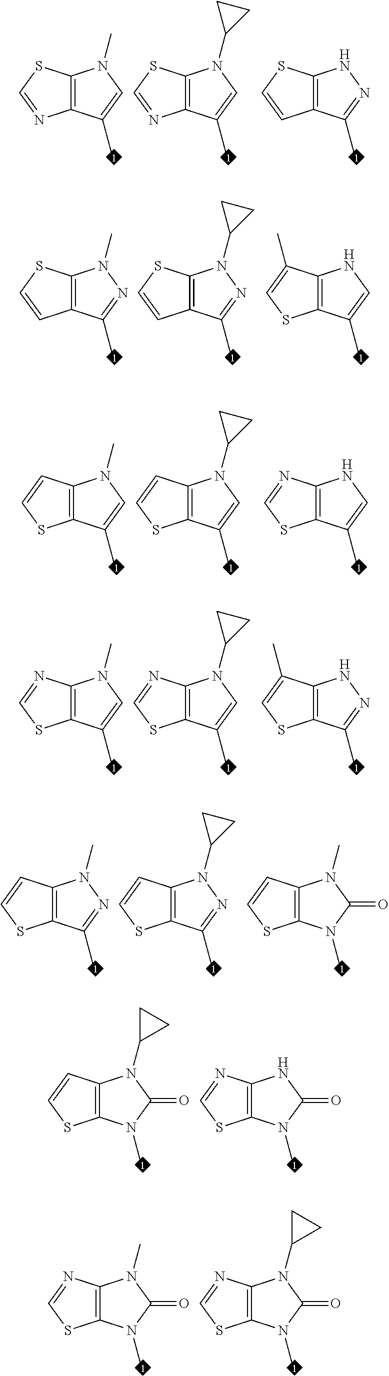

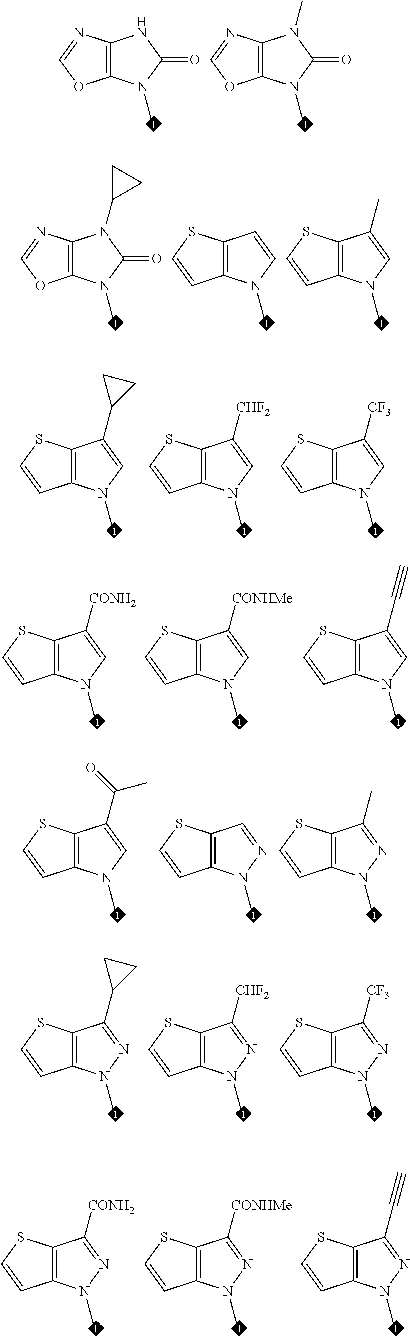

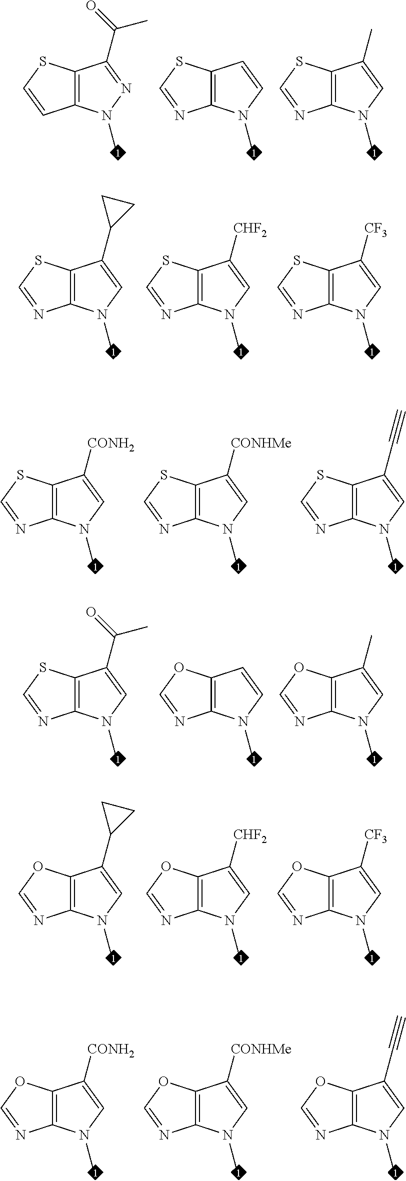

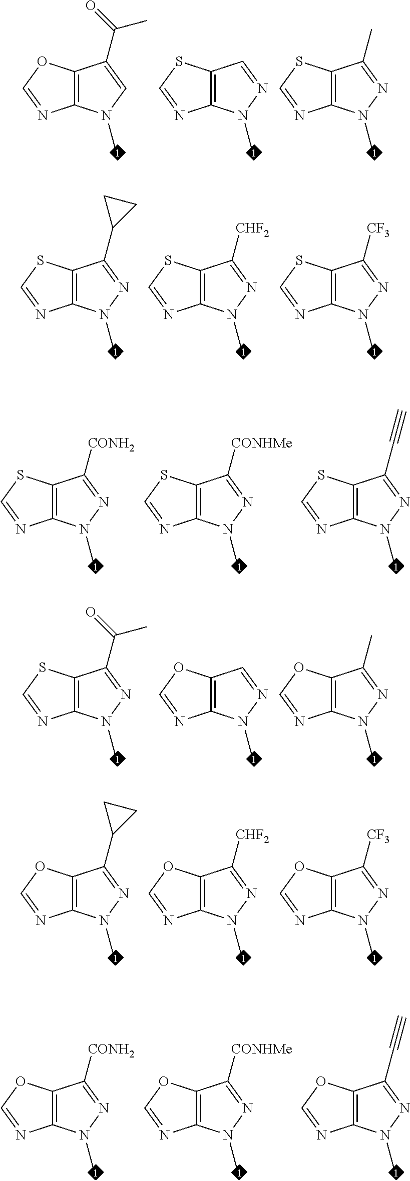

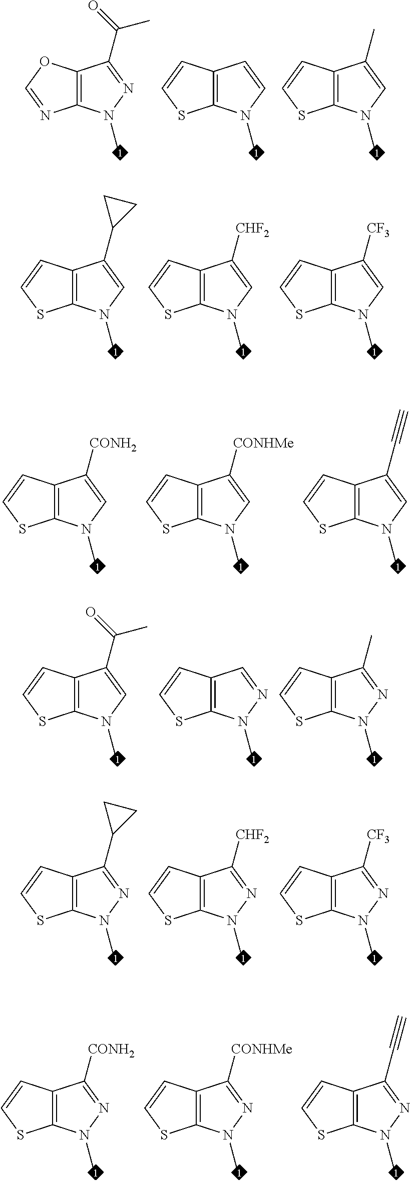

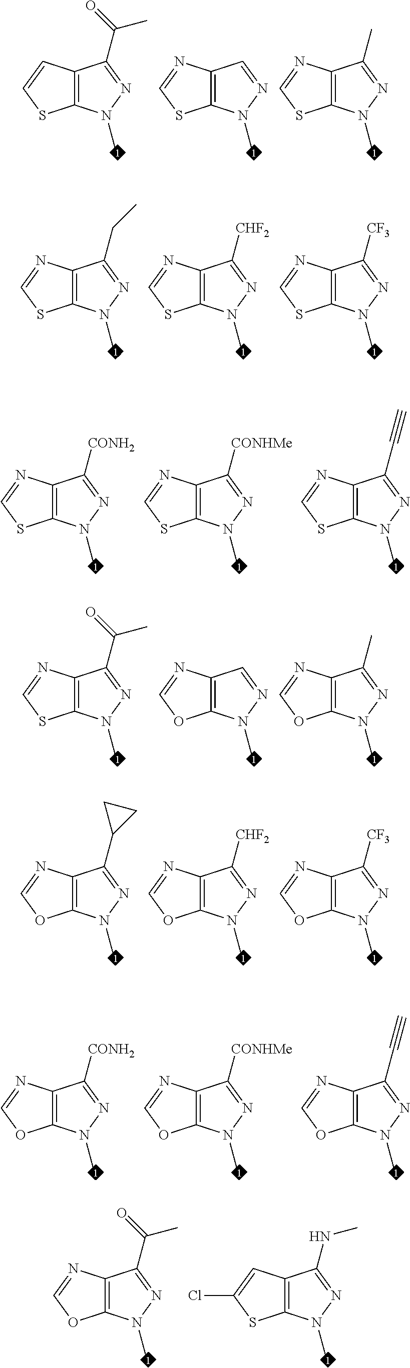

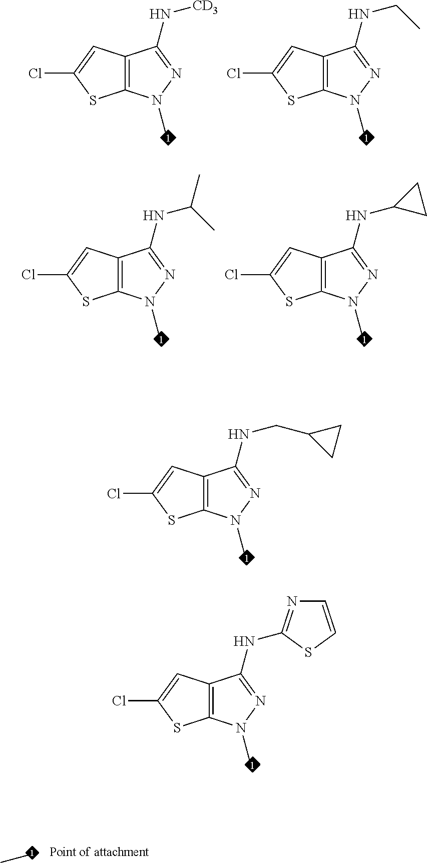

Representative examples of the 6.5 bicyclic azaaromatics which can be A^{4a} or A^{4b} are illustrated below, but the invention is not limited to these examples:

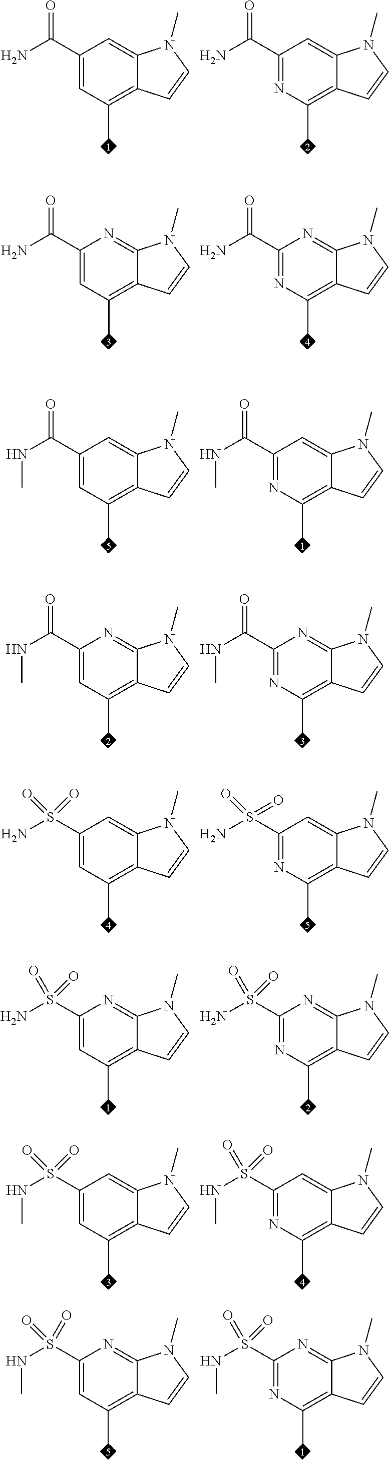

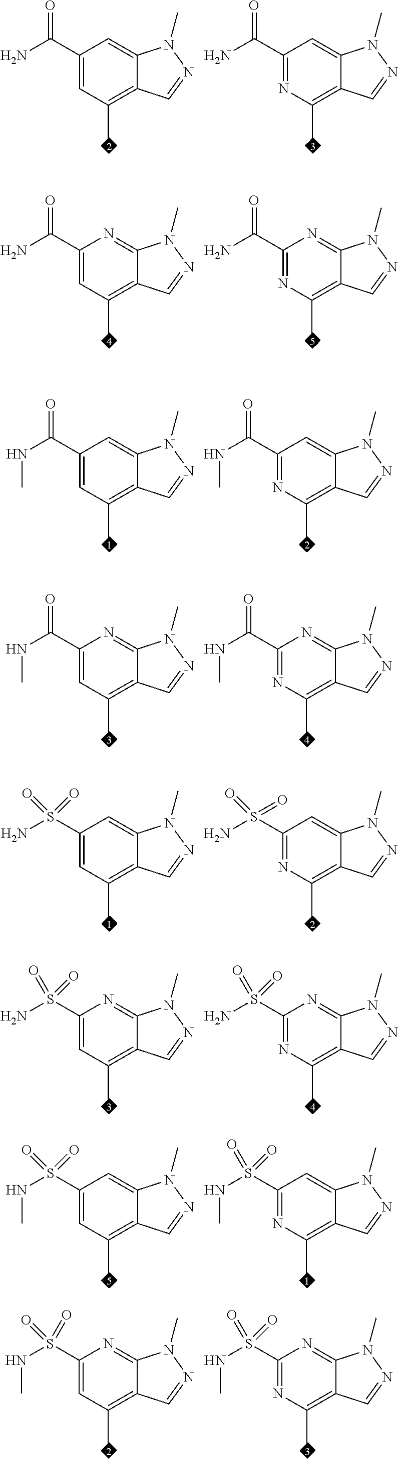

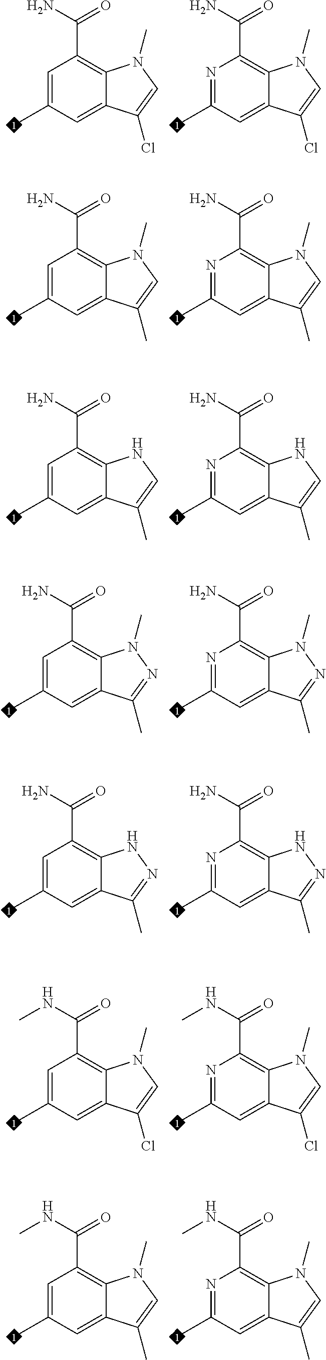

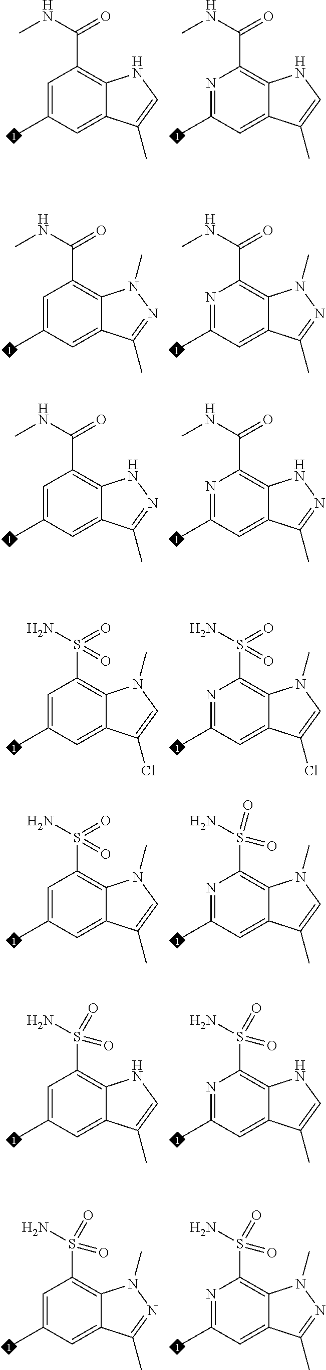

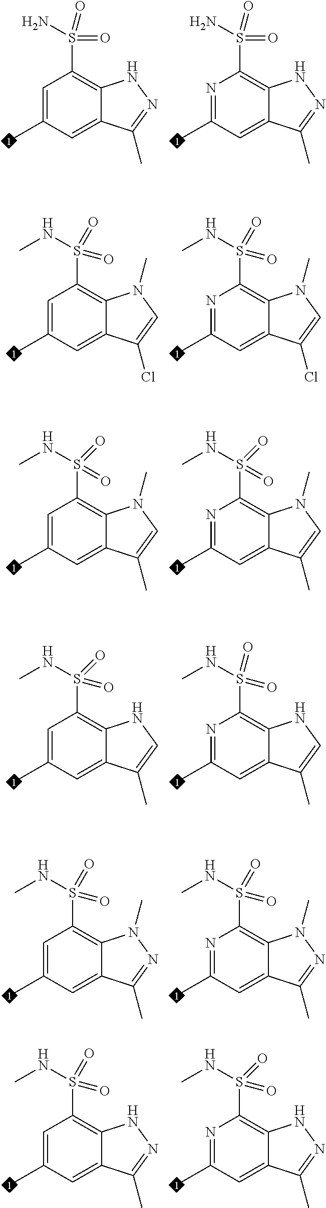

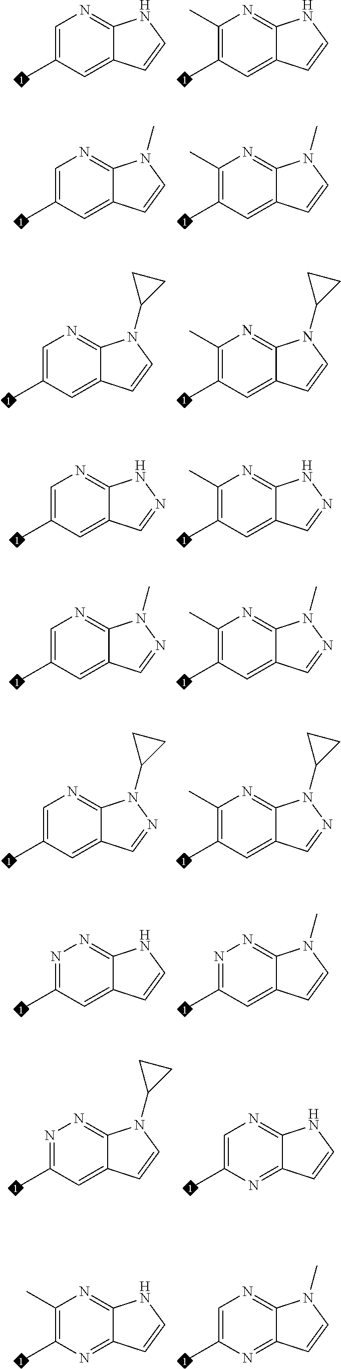

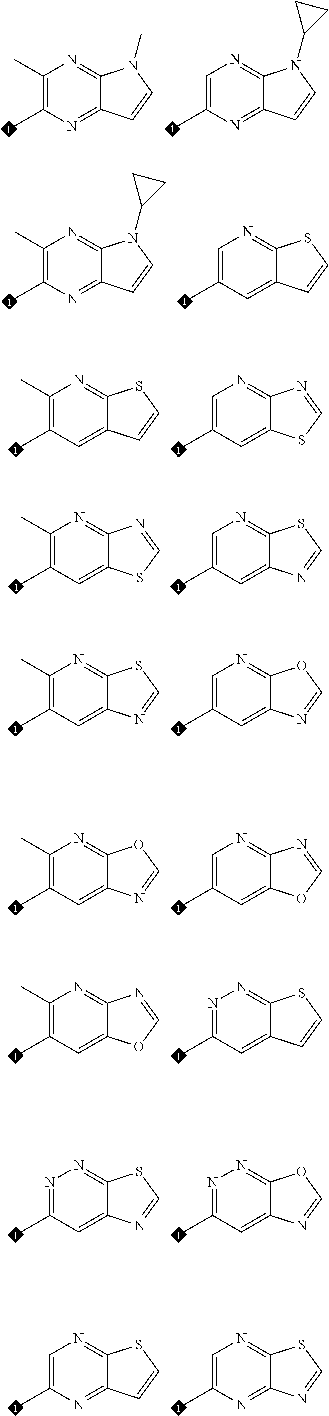

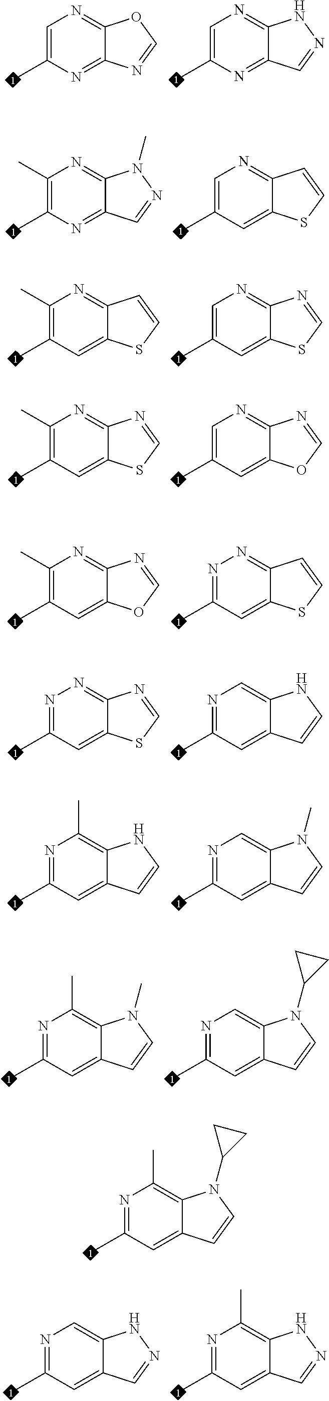

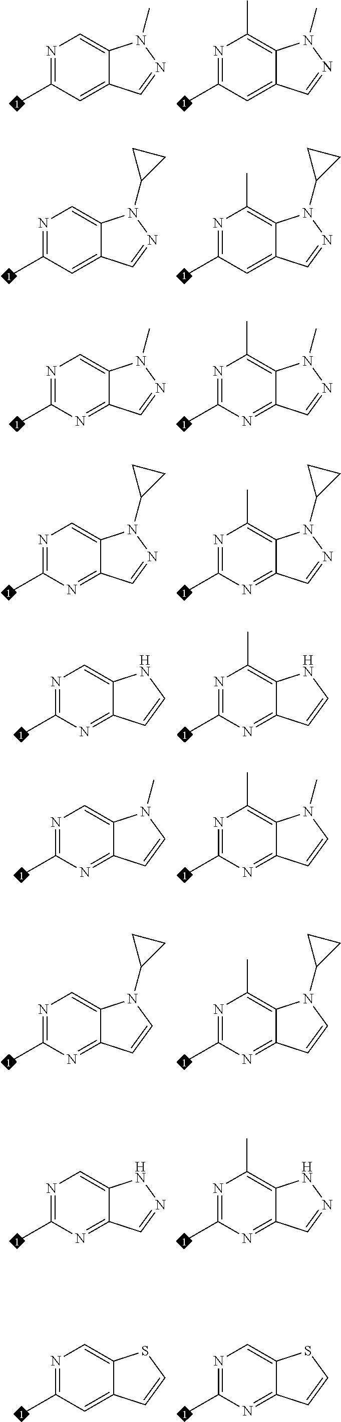

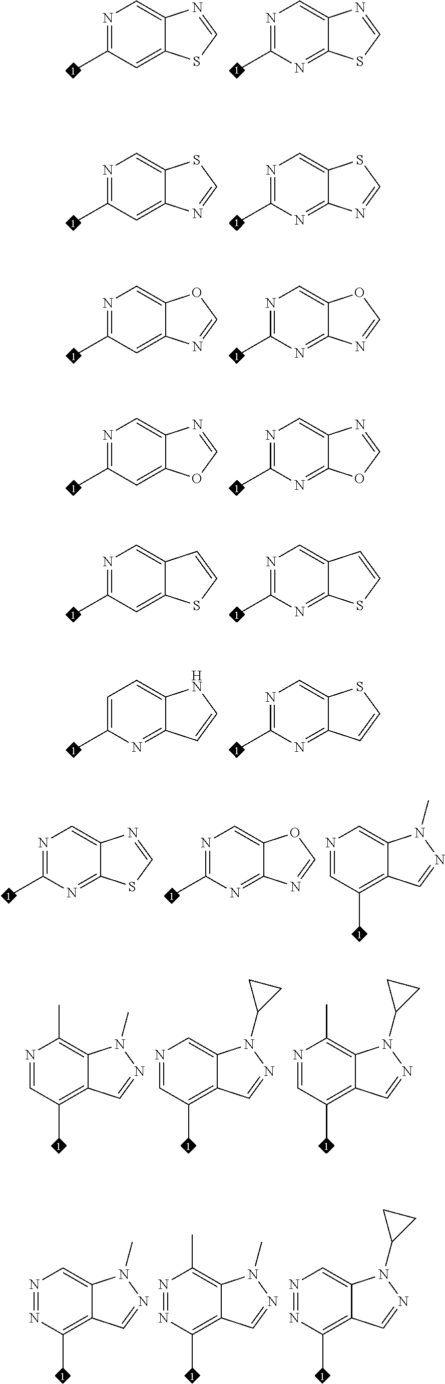

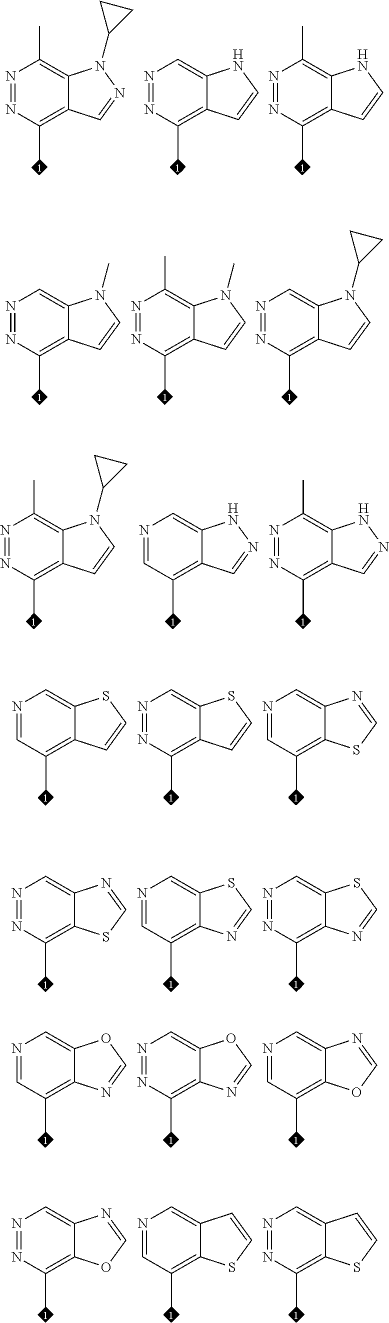

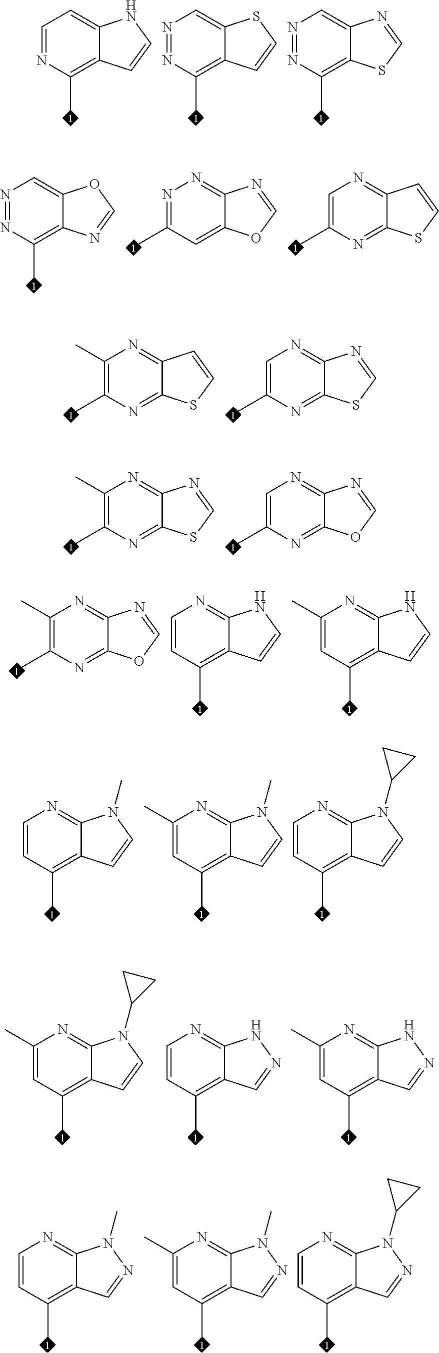

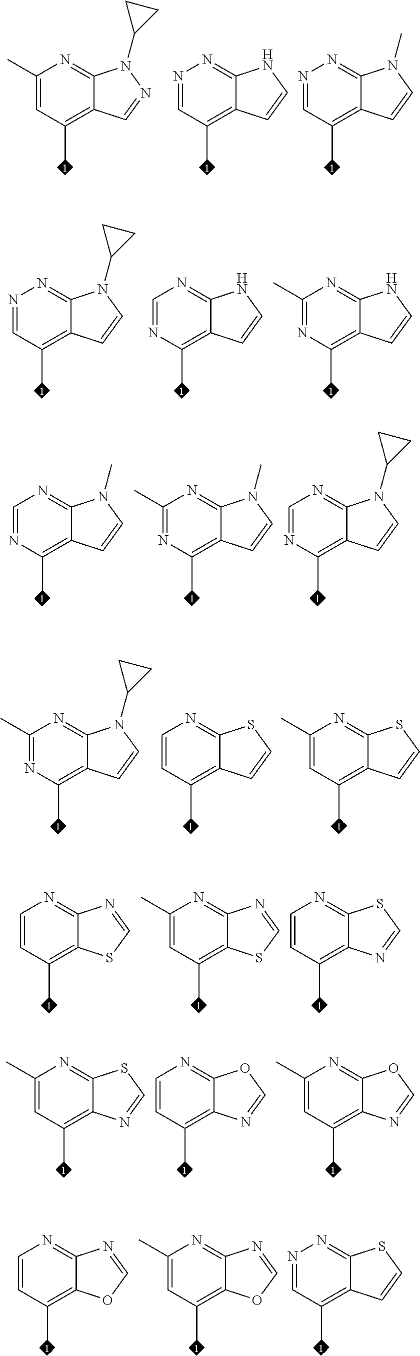

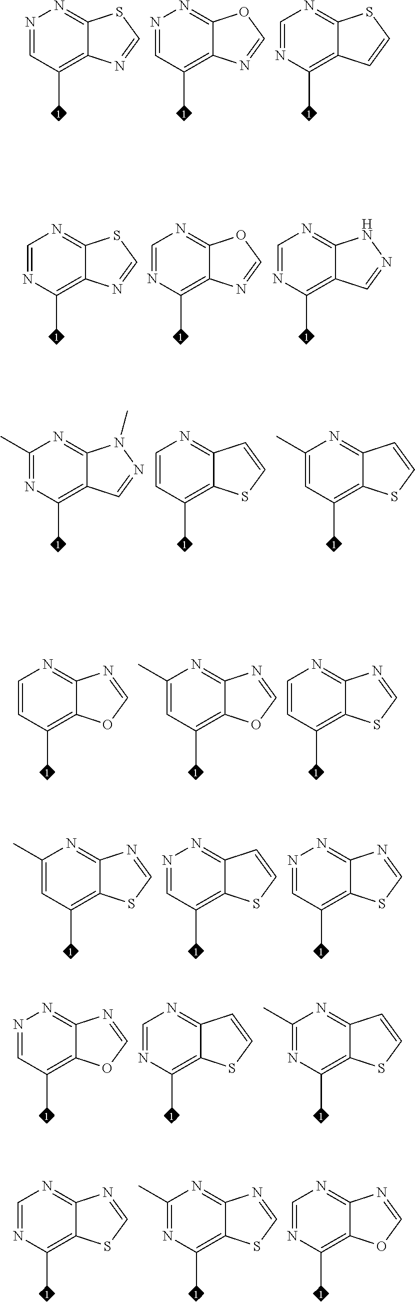

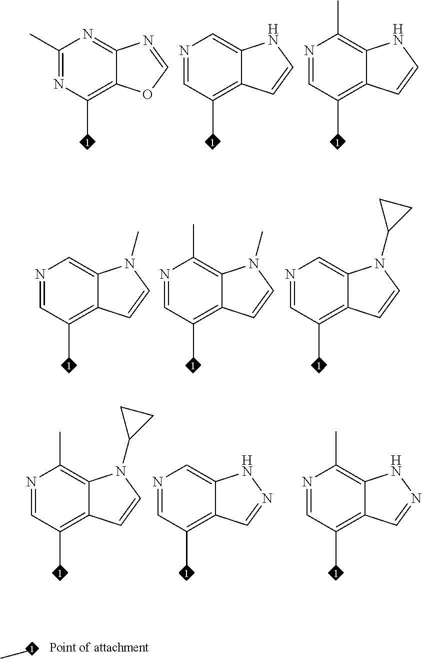

Representative examples of the A⁵ are illustrated below, but the invention is not limited to these examples:

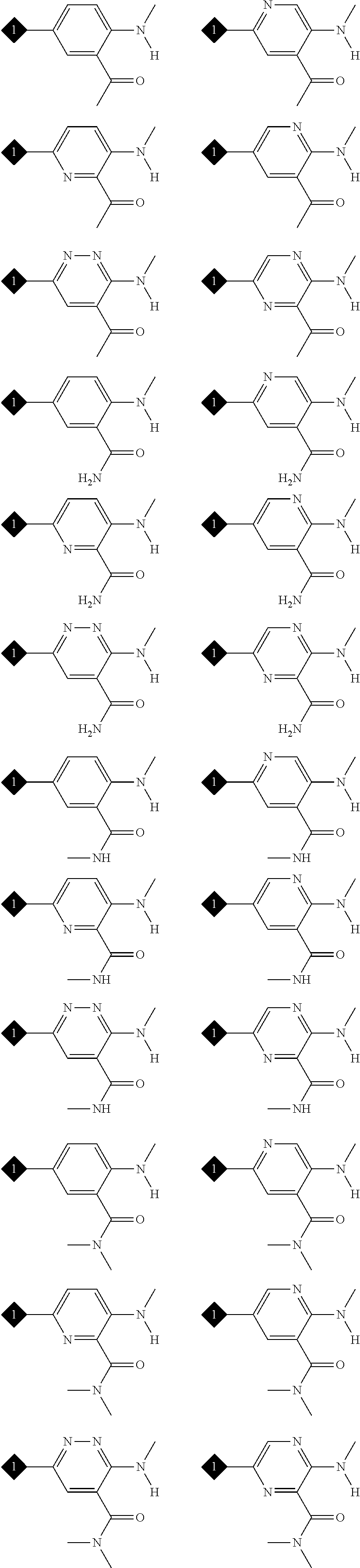

As used herein the term “replaced” in the context such as “a methylene unit is replaced by C═O” refers to exchange of functional group, for example, —CH₂— (methylene unit) is exchanged with —C(O)— (carbonyl group).

All alkyl, alkenyl, alkynyl, cycloalkyl, cycloalkenyl, monocyclic and bicyclic heterocycles, aryl (monocyclic and bicyclic), heteroaryl (monocyclic and bicyclic), cycloalkylalkyl, aralkyl, arylalkoxy, heteroaralkyl or heteroarylalkoxy groups (which include any C1-6 alkyl, C2-6 alkenyl, C2-6 alkynyl, C3-6 cycloalkyl, C4-6 cycloalkenyl, C6-12 bicycloalkyl, saturated monocyclic heterocycles of 4-12 atoms or saturated bicyclic heterocycles of 6-12 atoms, all C6-12 aryl monocycles or bicycles and heteroaryl monocycles or bicycles of 6-12 atoms) can be optionally substituted with multiple substituents independently chosen from halogen, hydroxy, oxo, hydroxylamino, oximino, hydrazino, hydrazono, cyano, nitro, azido, NR⁸R⁹, OC1-6 alkyl, OC3-6 alkenyl, OC3-6 alkynyl, C1-6 alkyl, OC3-8 cycloalkyl, OC3-8 cycloalkenyl, C1-6 acyl, C1-6 acyloxy, N(R⁸)COR⁴, CO₂R⁴, CONR⁸R⁹, NR⁸CONR⁸R⁹, NR⁸CO₂R⁴, OCO2R⁴, OCONR⁸R⁹, S(O)ₓR⁴, S(R⁴)(═O)═NR⁸, S(═O)(═NR⁸)NR⁸R⁹, SO₂NR⁸R⁹, NR⁸SO₂R⁴, NR⁸SO₂NR⁸R⁹, —NR⁸S(═O)(═NR⁸)R⁴, —N═S(═O)(R⁴)R⁴, —N═S(═O)(NR⁸R⁹)R⁴, ONR⁸R⁹, ON(R⁸)COR⁴, ONR⁸CONR⁸R⁹, ONR⁸CO₂R⁴, ONR⁸SO₂R⁴, ONR⁸SO₂NR⁸R⁹.

### Compounds

In one embodiment, the present invention relates to a compound of the formula (I):

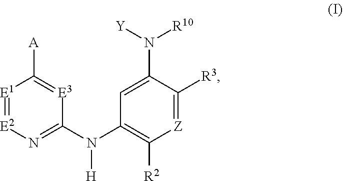

or a stereoisomer or a pharmaceutically acceptable salt, solvate, ester, tautomer, or prodrug thereof;
wherein,
A is

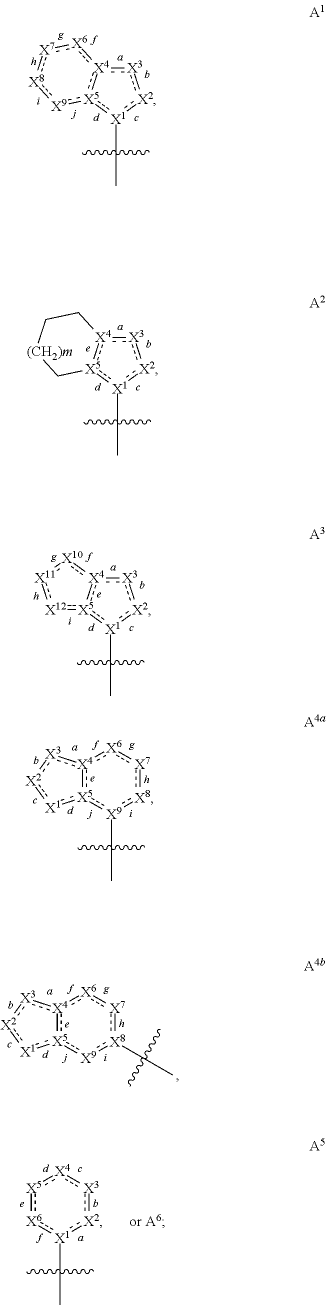

each of a, b, c, d, e, f, g, h, i and j are independently either (formal) double bonds or (formal) single bonds, and none of X¹, X², X³, X⁴, X⁵, X⁶, X⁷, X⁸, X⁹, X¹⁰, X¹¹, and X¹² has two (formal) double bonds attached thereto;
each of X¹, X², X³, X⁶, X⁷, X⁸, X⁹, X¹⁰, X¹¹, and X¹² is optionally substituted and is independently C or a heteroatom selected from the group consisting of N, S, and O; and the optional substituent is selected from the group consisting of ═O (oxo), ═S, ═NR¹³, (═O)₂, (O)(NR¹³), R⁴, and R¹³; or
alternatively, each of X¹, X², X³, X⁶, X⁷, X⁸, X⁹, X¹⁰, X¹¹, and X¹² is selected from the group consisting of: C, CH, CR⁴, C(R⁴)₂, CR¹³, CH₂, C═O, C═S, C═NR¹³, N, NR⁴, NR¹³, N(O), S, S(O), S(O)₂, S(═O)(═NR¹³), S(═NR¹³)₂, and O;
in A¹, A², A³, A^{4a}, and A^{4b}, each of X⁴ and X⁵ is independently C or N;
at least four of X¹, X², X³, X⁴, X⁵, X⁶, X⁷, X⁸, X⁹, X¹⁰, X¹¹, and X¹² are C, CR⁴, or C(R⁴)₂;
in A¹, A², A³ and A⁵, X¹ is C, CH or N;
in A^{4a}, X⁹ is C, CH or N;
in A^{4b}, X⁸ is C, CH or N;
in A^{4a} and A⁴b, X¹ is N, NR¹³, C(R⁴)₂, C(O), S(O)ₓ, S(═O)(═NR¹³), S(═NR¹³)₂, or CR⁴;
in A¹, A², A³, A^{4a}, and A⁴b, X² is N, NR¹³, C(R⁴)₂, S(O)ₓ, S(═O)(═NR¹³), S(═NR¹³)₂, C(O), or CR⁴;
in A¹, A², A³, A^{4a}, and A⁴b, X³ is N, NR¹³, C(R⁴)₂, C(O), S(O)ₓ, S(═O)(═NR¹³), S(═NR¹³)₂, or CR⁴;
in A⁵, at least three of X², X³, X⁴, X⁵ and X⁶ are C, C═O, CR⁴, or C(R⁴)₂;
E¹ and E² are independently C—R¹ or N with the proviso that E¹ and E² are not both N;
E³ and Z are independently CH or N;
Y is

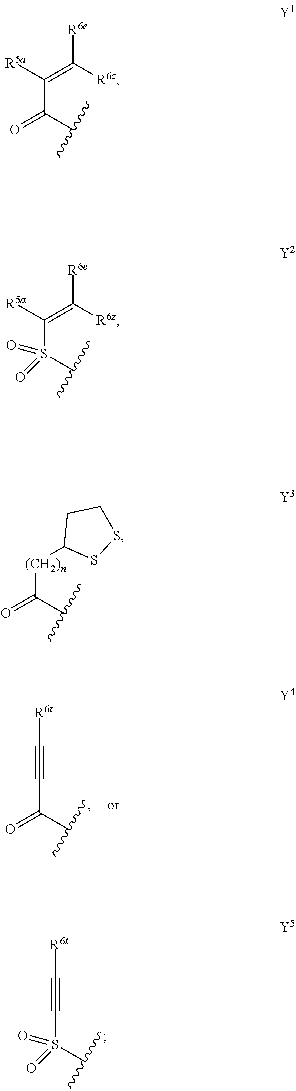

R¹ is independently selected from hydrogen, fluoro, chloro, bromo, methyl, ethyl, hydroxyl, methoxy, ethoxy, isopropoxy, —OCF₃, —OCH₂CF₃, —OCH₂CHF₂, ethenyl, ethynyl, CF₃, CHF₂, CHO, CH₂OH, CONH₂, CO₂Me, CONHMe, CONMe₂, and cyano;
R² is R¹⁰, —OCF₃, —OCHF₂, —OCF₂CF₃, —OCH₂CHF₂, —OCH₂CF₃, cyclopropyl, cyclopropoxy, methoxy, ethoxy, or isopropoxy;
R³ is C₂₋₆ alkenyl-R⁷, C₂₋₆ alkynyl-R⁷, N(R¹⁰)C₂₋₆ alkyl-NR¹⁰R¹⁰, N(R¹⁰)C₂₋₆ alkyl-R⁷, O(CH₂)ₚR⁷, N(R¹⁰)C(═O)(CH₂)ₚR⁷, C(R⁵)═C(R⁵)(CH₂)ₚR⁷, or R⁷;
each R⁴ is independently H, cyano, nitro, halo, —C₁₋₆ alkyl, —C₁₋₆ haloalkyl, C₁₋₆ acyl-C₁₋₆ alkyl-, R⁷—(CH₂)ₚC(═O)—C₁₋₆ alkyl-, carboxy-C₁₋₆ alkyl-, C₁₋₆ alkyloxycarbonyl-C₁₋₆ alkyl-, R⁷—(CH₂)ₚO—C(═O)—C₁₋₆ alkyl-, R⁸R⁹N—C(═O)C₁₋₆ alkyl-, R⁷—C₂₋₆alkyl-N(R¹⁰)—C(═O)C₁₋₆ alkyl-, —C₁₋₆ hydroxyalkyl, C₁₋₆ alkoxy-C₁₋₆ alkyl-, R⁷(CH₂)ₚOC₁₋₆ alkyl-, C₁₋₆ acyloxy-C₁₋₆ alkyl-, R⁷—(CH₂)ₚC(═O)O—C₁₋₆ alkyl-, C₁₋₆ alkoxy-C(═O)O—C₁₋₆ alkyl-, R⁷(CH₂)ₚO—C(═O)—OC₁₋₆ alkyl-, R⁸R⁹N—C(═O)OC₁₋₆ alkyl-, C₁₋₆ alkyl-N(R¹⁰)C(═O)O—C₁₋₆ alkyl-, R⁷(CH₂)ₚN(R¹⁰)—C(═O)O—C₁₋₆ alkyl-, R⁸R⁹N—C₁₋₆ alkyl-, R¹³R¹³N—C₁₋₆ alkyl-, R⁷—C₁₋₆ alkyl-, C₁₋₆ acylN(R¹⁰)—C₁₋₆ alkyl-, R⁷—C₁₋₆ acylN(R¹⁰)—C₁₋₆ alkyl-, R⁷—(CH₂)ₚC(═O)(N(R¹⁰)—C₁₋₆ alkyl-, R⁷—C₀₋₆ alkylC(═O)N(R¹⁰)—C₁₋₆ alkyl-, C₁₋₆ alkoxy-C(═O)N(R¹⁰)—C₁₋₆ alkyl-, R⁷—(CH₂)ₚOC(═O)N(R¹⁰)C₁₋₆ alkyl-, R⁸R⁹NC(═O)N(R¹)C₁₋₆ alkyl-, R¹⁰SO₂—N(R¹⁰)—C₁₋₆ alkyl-, R⁷—SO₂—N(R¹⁰)—C₁₋₆ alkyl-, C₁₋₆ alkylS(O)ₓ—C₁₋₆ alkyl-, R⁷—(CH₂)ₚS(O)ₓC₁₋₆ alkyl-, R⁷SO₂C₁₋₆ alkyl-, C₁₋₆ alkylS(═O)(═NR¹³)—C₁₋₆ alkyl-, C₁₋₆ haloalkyl S(═O)(═NR¹³)—C₁₋₆ alkyl-, C₁₋₆ alkylS(═NR¹³)(═NR¹³)—C₁₋₆ alkyl-, C₁₋₆ haloalkyl S(═NR¹³)(═NR¹³)—C₁₋₆ alkyl-, R⁷S(═O)(═NR¹³)C₁₋₆ alkyl-, R⁷S(═NR¹³)(═NR¹³)—C₁₋₆ alkyl-, —C₂₋₆ alkenyl, —C₂₋₆ haloalkenyl, R⁷—C₃₋₆ alkenyl-, C₁₋₆ alkoxy-C₃₋₆ alkenyl-, —C₂₋₆ alkynyl, —C₂₋₆ haloalkynyl, R⁷—C₂₋₆ alkynyl-, C₂₋₆ alkynyl-, C₁₋₆ acyl-, R⁷—(CH₂)ₚC(═O)—, R⁷—C₁₋₆ alkyl-C(═O)—, C₁₋₆ hydroxyalkyl-C(═O)—, C₁₋₆ alkoxy-C₁₋₆ alkyl-C(═O)—, C₁₋₆ alkylS(O)ₓ—C₁₋₆ alkyl-C(═O)—, carboxy, —C₁₋₆ alkoxycarbonyl, R⁷—(CH₂)ₚoxycarbonyl-, —C(═O)NR⁸R⁹, R⁷—(CH₂)ₚ—N(R¹⁰)—C(═O)—, hydroxyl, —C₁₋₆ alkoxy, —C₁₋₆ haloalkoxy, C₁₋₆ alkyl-N(R¹⁰)C(═O)—C₁₋₆ alkoxy-, R⁷(CH₂)ₚO—, R⁷(CH₂)ₚOC(═O)OC₂₋₆ alkoxy-, R⁷(CH₂)ₚN(R¹⁰)—C(═O)O—C₂₋₆ alkoxy-, R⁸R⁹N—C(═O)OC₂₋₆ alkoxy-, C₁₋₆ alkoxy-C(═O)N(R¹⁰)—C₂₋₆ alkoxy-, R⁷—(CH₂)ₚOC(═O)N(R¹⁰)C₂₋₆ alkoxy-, R⁸R⁹NC(═O)N(R¹⁰)C₂₋₆ alkoxy-, C₁₋₆ alkoxycarbonylC₁₋₆ alkoxy-, R⁷(CH₂)ₚ OC(═O)C₁₋₆ alkoxy-, C₁₋₆ acyloxy, R⁷—(CH₂)ₚC(═O)O—, —NR⁸R⁹, —NR¹³R¹³, R⁸R⁹N—C₂₋₆alkyl-N(R¹⁰)—, R⁷—C₂₋₆alkyl-N(R¹⁰)—, C₁₋₆ acyl-N(R¹⁰)—, C₁₋₆ alkoxycarbonyl-N(R¹⁰)—, R⁸R⁹N—C(═O)—N(R¹⁰)—, R⁷—C₁₋₆acyl-N(R¹⁰)—, C₁₋₆ alkylS(O)₂—N(R¹⁰)—, R¹⁰S(O)₂—N(R¹⁰)—, C₁₋₆ haloalkylS(O)₂—N(R¹⁰)—, R⁷SO₂—N(R¹⁰)—, thio, C₁₋₆ alkylS(O)ₓ—, C₁₋₆ haloalkylS(O)ₓ—, R⁷—(CH₂)ₚS(O)₂—, R⁷SO²—, C₁₋₆ alkyl-S(═O)(═NR¹³)—, C₁₋₆ haloalkyl-S(═O)(═NR¹³)—, C₁₋₆ alkylS(═NR¹³)(═NR¹³). C₁₋₆ haloalkyl-S(═NR¹³)(═NR¹³)—, R⁷S(═O)(═NR¹³)—, R⁷S(═NR¹³) (═NR¹³)—, C₆₋₁₂ aryl, C₆-C₁₂ aryl-C₁-C₆ alkyl-, 5-12 membered heteroaryl, 5-12 membered heteroaryl-C₁-C₆ alkyl-, C₃₋₈ cycloalkyl-, C₃₋₈ cycloalkyl-C₁-C₆ alkyl-, C₃₋₈ cycloalkenyl-, C₃₋₈ cycloalkenyl-C₁-C₆ alkyl-, 4-12 membered monocyclic or bicyclic heterocyclyl-, or 4-12 membered monocyclic or bicyclic heterocyclyl-C₁-C₆ alkyl-;
in R³, R⁵ is H, F, CF₃, CHF₂, or C₁-C₆ alkyl;
in Y¹ and Y², R^{5a} is H, F, Cl, CF₃, CHF₂, CF₂C₁₋₆ alkyl, CF₂CH₂NR⁸R⁹, CH₂NR⁸R⁹, CN, or C₁₋₆ alkyl;
in Y¹ and Y², R^{6e} is R¹⁰, H, F, aryl, heteroaryl, cycloalkyl, heterocycloalkyl, (CH₂)ₘCHR¹⁰R⁷, CF₂(CH₂)ₘCHR¹⁰R⁷, or C(R¹⁰)₂R⁷;
in Y⁴ and Y⁵, R^{6t} is C₁₋₆ alkyl, C₃₋₆ cycloalkyl, aryl, heteroaryl, heterocycloalkyl, (CH₂)ₘCHR¹⁰R⁷, C(R¹⁰)₂R⁷;
in Y¹ and Y², R^{6z} is H, F, Cl, CF₃, CHF₂, CF₂C₁₋₆ alkyl or C₁₋₆ alkyl; or
alternatively in Y¹ and Y², R^{6e} and R^{6z}, taken together, form R^{6e}R^{6z}C═; or
alternatively in Y¹ and Y², R^{6e} and R^{6z}, taken together with the sp² carbon atom to which both are attached, form an alicyclic ring of 4 to 7 members wherein one of the ring atoms are optionally replaced by NR⁸, O, S(O)ₓ, S(═O)(═NR⁸), P═O, P(═O)(OR⁸), OP(═O)(OR⁸)O, and the alicyclic ring is optionally substituted with one or more substituents selected from the group consisting of halogen, oxo, OH, OR⁸, and NR⁸R⁹;

R⁷ is OH, NR⁸R⁹, O(CH₂)_{q}NR⁸R⁹, C₁₋₆ alkoxy, C₁₋₆ alkoxy-C₁₋₆ alkoxy, C₂₋₆ hydroxyalkoxy, oxetanyl, oxetanyloxy, oxetanylamino, oxolanyl, oxolanyloxy, oxolanylamino, oxanyl oxanyloxy, oxanylamino, oxepanyl, oxepanyloxy, oxepanylamino, azetidinyl, azetidinyloxy, azetidylamino, pyrrolidinyl, pyrolidinyloxy, pyrrolidinylamino, piperidinyl, piperidinyloxy, piperidinylamino, azepanyl, azepanyloxy, azepanylamino, dioxolanyl, dioxanyl, morpholino, thiomorpholino, thiomorpholino-S,S-dioxide, piperazino, dioxepanyl, dioxepanyloxy, dioxepanylamino, oxazepanyl, oxazepanyloxy, oxazepanylamino, diazepanyl, diazepanyloxy, diazepanylamino, (3R)-3-(dimethylamino)pyrrolidin-1-yl, (3S)-3-(dimethylamino)pyrrolidin-1-yl, 3-(dimethylamino)azetidin-1-yl, [2-(dimethylamino)ethyl](methyl)amino, [2-(methylamino)ethyl](methyl)amino, 5-methyl-2,5diazaspiro[3.4]oct-2-yl, (3aR,6aR)-5-methylhexa-hydro-pyrrolo[3,4-b]pyrrol-1(2H)-yl, I-methyl-1,2,3,6-tetrahydropyridin-4-yl, 4-methylpiperizin-1-yl, 4-[2(dimethylamino)-2-oxoethyl]piperazin-1-yl, methyl[2-(4-methylpiperazin-1yl)ethyl]amino, methyl[2-(morpholin-4-yl)ethyl]amino, 1-amino-1,2,3,6tetrahydropyridin-4-yl, 4-[(2S)-2-aminopropanoyl]piperazin-1-yl, all of which may be optionally substituted with OH, OR¹⁰, oxo, halogen, R¹⁰, CH₂OR¹⁰, or CH₂NR⁸R⁹;

R⁸ and R⁹ are independently H, —CD₃, C₁₋₆ alkyl, C₁₋₆ haloalkyl, C₃₋₆ alkenyl, C₃₋₆ haloalkenyl, C₃₋₆ alkynyl, C₃-C₆ haloalkynyl, C₃₋₈ cycloalkyl, C₃₋₈ cycloalkyl-C₁-C₆ alkyl-, C₃₋₈ halocycloalkyl, C₃₋₈ halocycloalkyl-C₁-C₆ alkyl-, C₃₋₈ cycloalkenyl, C₃₋₈ cycloalkenyl-C₁-C₆ alkyl-, C₃₋₈ halocycloalkenyl, C₃₋₈ halocycloalkenyl-C₁-C₆ alkyl-, C₁-C₆ acyl, C₁-C₆ acyl-C₁-C₆ alkyl-, 4-12 membered monocyclic or bicyclic heterocyclyl, 4-12 membered monocyclic or bicyclic heterocyclyl-C₁-C₆ alkyl-, C₆-C₁₂ aryl, C₆-C₁₂ aryl-C₁-C₆ alkyl-, 5-12 membered heteroaryl, or 5-12 membered heteroaryl-C₁-C₆ alkyl-; and R⁸ and R⁹ may be further independently substituted with up to three substituents chosen from hydroxyl, C₁₋₆ alkoxy, C₁₋₆ hydroxyalkyl, C₁₋₆ alkoxy-C₁₋₆ alkyl, C₁₋₆ alkoxy-C₁₋₆ alkoxy, C₂₋₆ hydroxyalkoxy, oxo, thiono, cyano or halo; or
alternatively, R⁸ and R⁹, taken together with the N atom to which they are both attached, form a heterocyclic ring of 4-7 members, containing up to one other heteroatom chosen from O, S, or NR¹¹, and the heterocyclic ring is optionally substituted with up to three substituents chosen from hydroxyl, C₁₋₆ alkoxy, C₁₋₆ hydroxyalkyl, C₁₋₆ alkoxy-C₁₋₆ alkyl, C₁₋₆ alkoxy-C₁₋₆ alkoxy, C₂₋₆ hydroxyalkoxy, oxo, thiono, cyano or halo;
each R¹⁰ is independently H, —CD₃, C₁₋₆ alkyl, C₃₋₆ cycloalkyl, C₂₋₆ hydroxyalkyl, C₁₋₆ alkoxy-C₁₋₆ alkyl or C₂₋₆ alkyl-NR⁸R⁹;
each R¹¹ is independently hydrogen, C₁-C₆ alkyl, C₃-C₆ alkenyl, C₃-C₆ alkynyl, C₃-C₆ cycloalkyl, C₆-C₁₂ aryl, 4-12 membered heterocyclyl, or 5-12 membered heteroaryl; or
alternatively, two R¹¹, taken together with the heteroatom(s) attached thereto, form a 5-8 membered heterocyclyl ring, which is optionally substituted with up to three substituents selected from hydroxyl, C₁₋₆ alkoxy, C₁₋₆ hydroxyalkyl, C₁₋₆ alkoxy-C₁₋₆ alkyl, C₁₋₆ alkoxy-C₁₋₆ alkoxy, C₂₋₆ hydroxyalkoxy, oxo, thiono, cyano and halo;
each R¹³ is independently H, —CD₃, cyano, —C₁₋₆ alkyl, —C₁₋₆ haloalkyl, C₁₋₆ acyl-C₁₋₆ alkyl-, R⁷—(CH₂)ₚC(═O)—C₁₋₆ alkyl-, carboxy-C₁₋₆ alkyl-, C₁₋₆ alkyloxycarbonyl-C₁₋₆ alkyl-, R⁷—(CH₂)ₚO—C(═O)—C₁₋₆ alkyl-, R⁸R⁹N—C(═O)C₁₋₆ alkyl-, R⁷—C₂₋₆alkyl-N(R¹⁰)—C(═O)C₁₋₆ alkyl-, —C₂₋₆ hydroxyalkyl, C₁₋₆ alkoxy-C₂₋₆ alkyl-, R⁷(CH₂)ₚOC₂₋₆ alkyl-, C₁₋₆ acyloxy-C₂₋₆ alkyl-, R⁷—(CH₂)ₚC(═O)O—C₂₋₆ alkyl-, C₁₋₆ alkoxy-C(═O)O—C₂₋₆ alkyl-, R⁷(CH₂)ₚO—C(═O)—OC₂₋₆ alkyl-, R⁸R⁹N—C(═O)OC₂₋₆ alkyl-, C₁₋₆ alkyl-N(R¹⁰)C(═O)O—C₂₋₆ alkyl-, R⁷(CH₂)ₚN(R¹⁰)—C(═O)O—C₂₋₆ alkyl-, R⁸R⁹N—C₂₋₆ alkyl-, R⁷—C₂₋₆ alkyl-, C₁₋₆ acylN(R¹⁰)—C₂₋₆ alkyl-, R⁷—C₁₋₆ acylN(R¹⁰)—C₂₋₆ alkyl-, R⁷—(CH₂)ₚC(═O)N(R¹⁰)—C₂₋₆ alkyl-, R⁷—C₀₋₆ alkylC(═O)N(R¹⁰)—C₂₋₆ alkyl-, C₁₋₆ alkoxy-C(═O)N(R¹⁰)—C₂₋₆ alkyl-, R⁷—(CH₂)ₚOC(═O)N(R¹⁰)C₂₋₆ alkyl-, R⁸R⁹NC(═O)N(R¹⁰)C₂₋₆ alkyl-, R¹⁰SO₂—N(R¹⁰)—C₂₋₆ alkyl-, R⁷—SO₂—N(R¹⁰)—C₂₋₆ alkyl-, C₁₋₆ alkylS(O)ₓ—C₂₋₆ alkyl-, R⁷—(CH₂)ₚS(O)ₓC₂₋₆ alkyl-, R⁷SO₂C₂₋₆ alkyl-, C₁₋₆ alkylS(═O)(═NR¹⁰)—C₂₋₆ alkyl-, C₁₋₆ haloalkyl S(═O)(═NR¹⁰)—C₂₋₆ alkyl-, C₁₋₆ alkylS(═NR¹⁰)(═NR¹⁰)—C₂₋₆ alkyl-, C₁₋₆ haloalkyl S(═NR¹⁰)(═NR¹⁰)—C₂₋₆ alkyl-, R⁷S(═O)(═NR¹⁰)C₂₋₆ alkyl-, R⁷S(═NR¹³)(═NR¹³)—C₂₋₆ alkyl-, —C₃₋₆ alkenyl, —C₃₋₆ haloalkenyl, R⁷—C₄₋₆ alkenyl-, C₁₋₆ alkoxy-C₄₋₆ alkenyl-, —C₂₋₆ alkynyl, —C₂₋₆ haloalkynyl, R⁷—C₂₋₆ alkynyl-, C₂₋₆ alkynyl-, C₁₋₆ acyl-, R⁷—(CH₂)ₚC(═O)—, R⁷—C₁₋₆ alkyl-C(═O)—, C₁₋₆ hydroxyalkyl-C(═O)—, C₁₋₆ alkoxy-C₁₋₆ alkyl-C(═O)—, C₁₋₆ alkylS(O)ₓ—C₁₋₆ alkyl-C(═O)—, —C₁₋₆ alkoxycarbonyl, R⁷—(CH₂)ₚoxycarbonyl-, —C(═O)NR⁸R⁹, R⁷—(CH₂)ₚ—N(R¹⁰)—C(═O)—, hydroxyl, —C₁₋₆ alkoxy, —C₁₋₆ haloalkoxy, C₁₋₆ alkyl-N(R¹⁰)C(═O)—C₁₋₆ alkoxy-, R⁷(CH₂)ₚO—, R⁷(CH₂)ₚOC(═O)OC₂₋₆ alkoxy-, R⁷(CH₂)ₚN(R¹⁰)—C(═O)O—C₂₋₆ alkoxy-, R⁸R⁹N—C(═O)OC₂₋₆ alkoxy-, C₁₋₆ alkoxy-C(═O)N(R¹⁰)—C₂₋₆ alkoxy-, R⁷—(CH₂)ₚOC(═O)N(R¹⁰)C₂₋₆ alkoxy-, R⁸R⁹NC(═O)N(R)C₂₋₆ alkoxy-, C₁₋₆ alkoxycarbonyl-C₁₋₆ alkoxy-, R⁷(CH₂)ₚOC(═O)C₁₋₆ alkoxy-, —C₁₋₆ acyloxy, R⁷—(CH₂)ₚC(═O)O—, —NR⁸R⁹, R⁸R⁹N—C₂₋₆alkyl-N(R¹⁰)—, R⁷—C₂₋₆alkyl-N(R¹⁰)—, C₁₋₆ acyl-N(R¹⁰)—, C₁₋₆ alkoxycarbonyl-N(R¹⁰)—, R⁸R⁹N—C(═O)—N(R¹⁰)—, R⁷—C₁₋₆acyl-N(R¹⁰)—, C₁₋₆ alkylS(O)₂—N(R¹⁰)—, R¹⁰S(O)₂—N(R¹⁰)—, C₁₋₆ haloalkylS(O)₂—N(R¹⁰)—, R⁷SO₂—N(R¹⁰)—, C₁₋₆ alkylS(O)ₓ—, C₁₋₆ haloalkylS(O)ₓ—, R⁷—(CH₂)ₚS(O)₂, R⁷SO₂—, C₁₋₆ alkyl-S(═O)(═NR¹⁰)—, C₁₋₆ haloalkyl-S(═O)(═NR¹⁰)—, C₆₋₁₂ aryl, C₆-C₁₂ aryl-C₁-C₆ alkyl-, 5-12 membered heteroaryl, 5-12 membered heteroaryl-C₁-C₆ alkyl-, C₃₋₈ cycloalkyl-, C₃₋₈ cycloalkyl-C₁-C₆ alkyl-, C₃₋₈ cycloalkenyl-, C₃₋₈ cycloalkenyl-C₁-C₆ alkyl-, 4-12 membered monocyclic or bicyclic heterocyclyl-, or 4-12 membered monocyclic or bicyclic heterocyclyl-C₁-C₆ alkyl-; or
alternatively, two R⁴, two R¹³, or R¹³ and R⁴, taken together with atoms attached thereto, form a ring of 5-7 members, which may be aromatic or partially saturated, and which may contain up to two heteroatoms chosen from N, O and S; and the 5-7 member ring is optionally further substituted by is selected from the group consisting of ═O (oxo), ═S, ═NR¹³, (═O)₂, (O)(NR¹³), R⁴, and R¹³;
A⁶ is selected from:

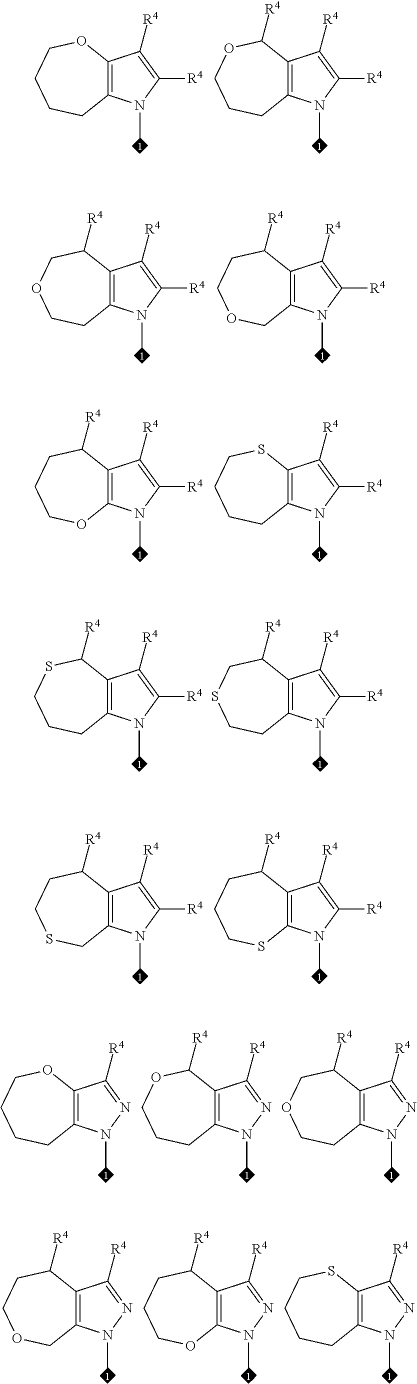

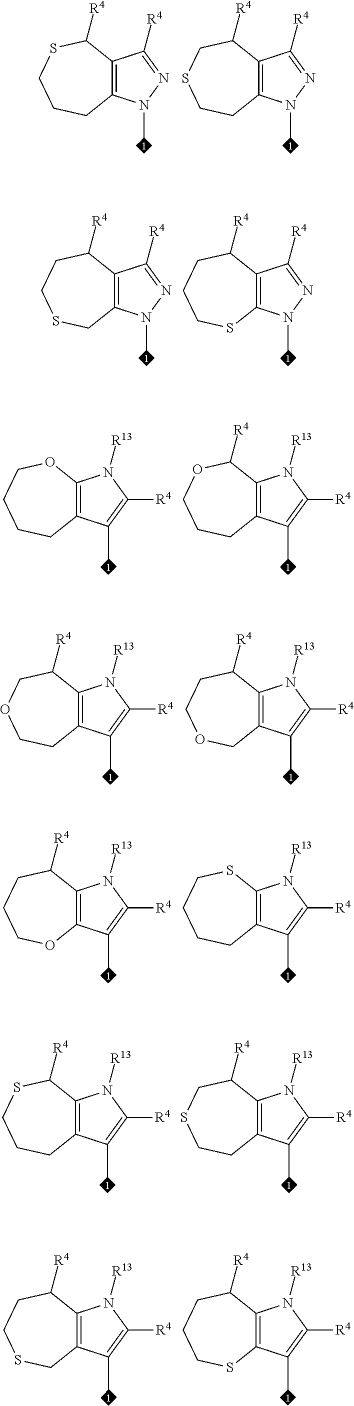

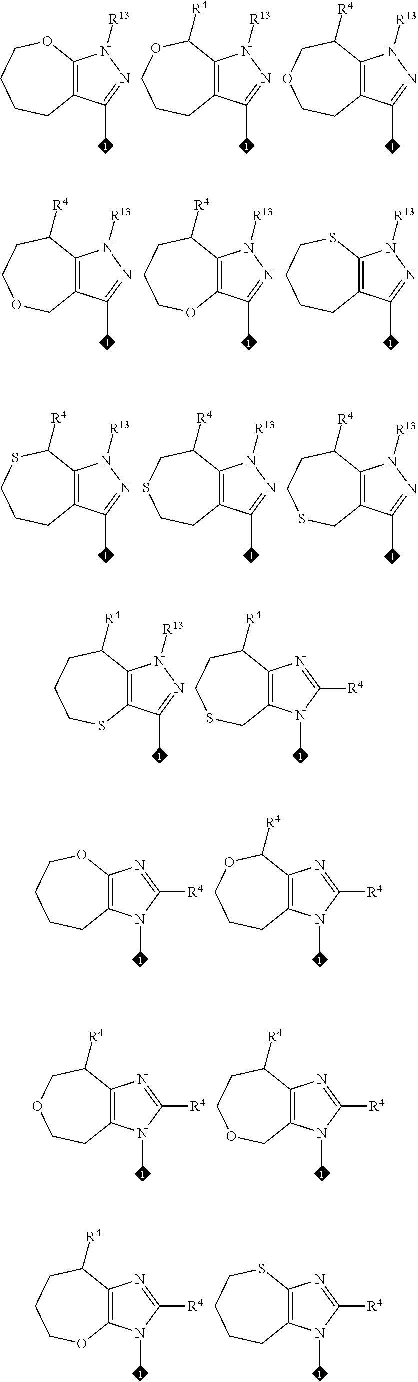

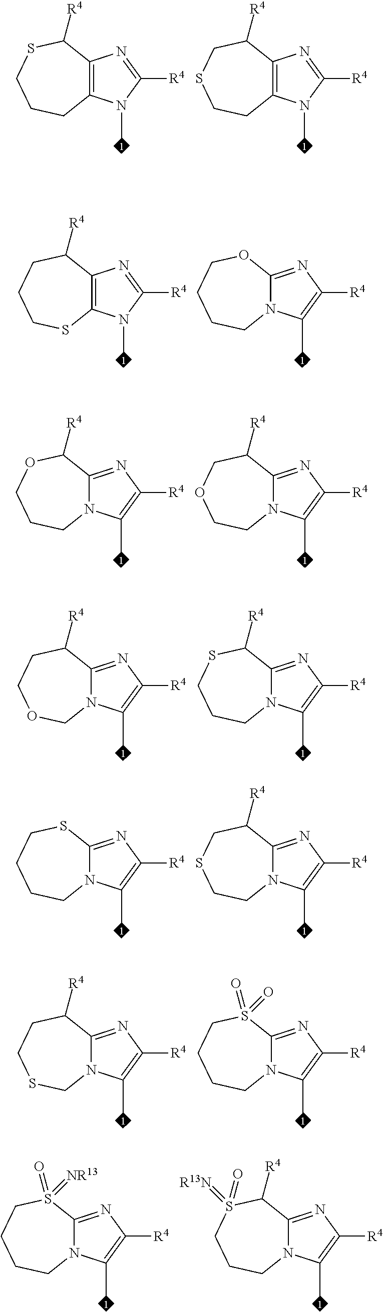

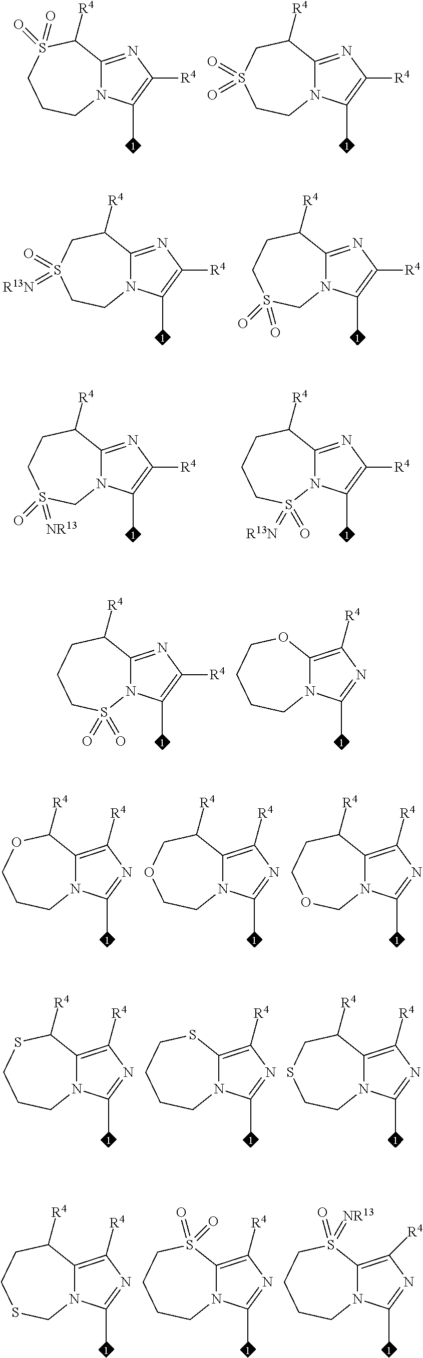

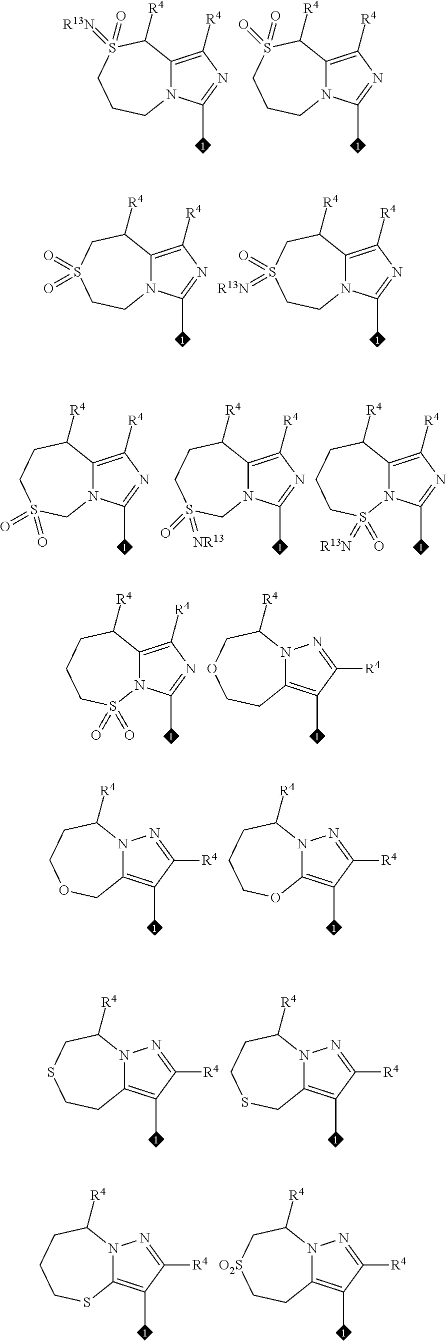

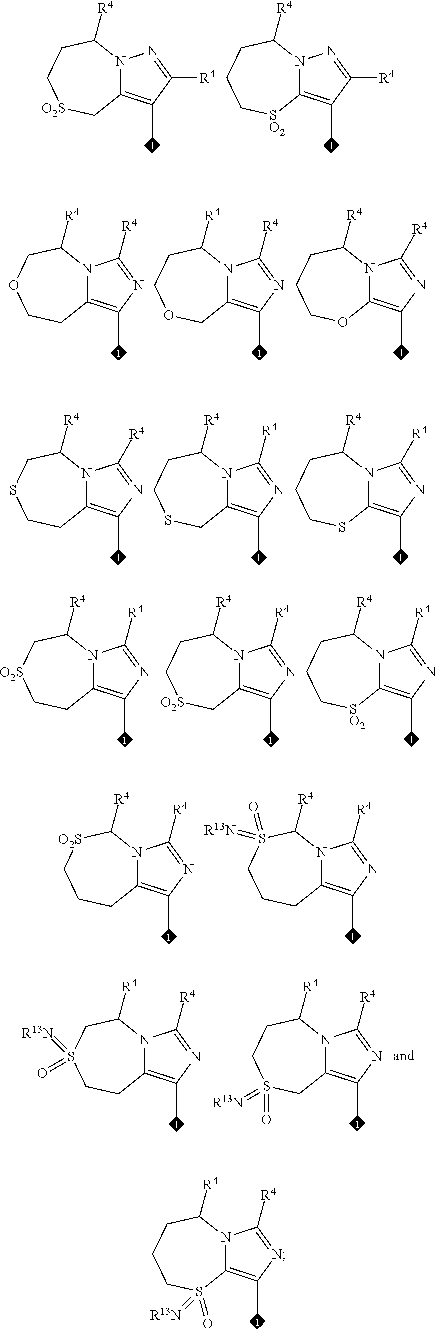

A² is optionally substituted with R⁴;
m is 0, 1, 2, or 3;
n=1, 2, or 3;
p=0, 1, 2, 3, or 4;
q=2, 3, or 4; and
x=0, 1, or 2.

In one embodiment of formula (I), each of X¹, X², X³, X⁶, X⁷, X⁸, X⁹, X¹⁰, X¹¹, and X¹² is selected from the group consisting of: C, CH, CR⁴, C(R⁴)₂, CR¹³, CH₂, C═O, C═S, C═NR¹³, N, NR⁴, NR¹³, N(O), S, S(O), S(O)₂, S(═O)(═NR¹³), S(═NR¹³)₂, and O.

In one embodiment of formula (I), if any of X², X³, X⁶, X⁷, X⁸, X⁹, X¹⁰, X¹¹, and X¹² is NR¹³, O, S, C═O, C═NR¹³, S═O or SO₂, none of the abovementioned bonds to said atom is a (formal) double bond; and at least four of X¹, X², X³, X⁴, X⁵, X⁶, X⁷, X⁸, X⁹, X¹⁰, X¹¹, and X¹² are C, CR⁴, or C(R⁴)₂.

In one embodiment of formula (I), each X¹, X², X³, X⁶, X⁷, X⁸, X⁹, X¹⁰, X¹¹, and X¹² is optionally substituted and is independently C or a heteroatom selected from the group consisting of N, S, O, and the functional groups of C═O, C═NR¹³, SO₂ or S(O)(NR¹³);

In one embodiment of formula (I), in A¹ A², A³, A^{4a}, and A^{4b}, no more than four, and no less than two of X¹, X², X³, X⁴, and X⁵ can be C, CR⁴, or C(R⁴)₂.

In one embodiment of formula (I), in A¹, A² and A³, if X¹, X⁴ and X⁵ are all C, then one of X² and X³ is O, C═O, SO₂, N, NR¹³ or S.

In one embodiment of formula (I), in A¹, A² and A³, if X¹ is N, X² is C═O, SO₂, C═NR¹³, NR¹³ or C═S, and X⁴ and X⁵ are both C, then X³ is C(R⁴)₂, O, NR¹³, C═O or S.

In one embodiment of formula (I), in A¹, A² and A³, if X¹ is C, and X² and X³ are CR⁴ or N, one of X⁴ and X⁵ may be N, but if X¹ is N, or if one of X² or X³ is not N or CR⁴, both X⁴ and X⁵ are C.

In one embodiment of formula (I), in A^{4a}, and A^{4b} at least one of X¹, X² and X³ is CR⁴ or N, and one of X⁴ and X⁵ is C or N, and the other is C.

In one embodiment of formula (I), in A², the methylene units in the non-aromatic ring are optionally substituted with up to three independent R⁴; and optionally up to two of the methylene units are independently replaced by C═O, C(R⁴)₂, NR¹⁰, O or S(O)ₓ.

In one embodiment of formula (I), in A¹X⁶, X⁷, X⁸, and X⁹ may be CR⁴, N, NR¹³, C(R¹)₂, C(O), or S(O)ₓ with the proviso that at least two of them are CR⁴, C(═O), C(═NR¹³) or N.

In one embodiment of formula (I), in A³, if X⁴ or X⁵ is N, then X¹⁰, X¹¹, and X¹² are independently N or CR⁴, with the proviso that at most two of X¹⁰, X¹¹, and X¹² are N.

In one embodiment of formula (I), in A³, if X⁴ and X⁵ are C, one of X¹⁰, X¹¹, and X¹² is NR¹⁰, O or S, then the remaining two are independently CR⁴ or N.

In one embodiment of formula (I), in A^{4a} and A^{4b} X⁶ and X⁷ may be CR⁴, N, NR¹³, C(R¹)₂, C(O), or S(O)ₓ with the proviso that at least two of them are CR⁴, C(═O), C(═NR¹³) or N.

In one embodiment of formula (I), in A^{4a}, X⁹ is C, CH or N.

In one embodiment of formula (I), in A^{4b}, X⁸ is C, CH or N.

In one embodiment of formula (I), in A⁵, at least three of X², X³, X⁴, X⁵ and X⁶ are C, C═O, CR⁴, or C(R⁴)₂. In one embodiment of formula (I), in A⁵, X¹ is C, CH or N.

In one embodiment of formula (I), when Z is CH, then A is not 4,5,6,7-tetrahydropyrazolo[1,5-a]pyridin-3-yl, 1H-indol-3-yl, 1-methyl-1H-indol-3-yl, or pyrazolo[1,5-a]pyridin-3-yl.

In one embodiment of formula (I), Z is N. In another embodiment, Z is CH.

In another embodiment of formula (I), R³ is selected from the group consisting of (3R)-3-(dimethylamino)pyrrolidin-1-yl, (3 S)-3-(dimethyl-amino)pyrrolidin-1-yl, 3-(dimethylamino)azetidin-1-yl, [2-(dimethylamino)ethyl]-(methyl)amino, [2-(methylamino)ethyl](methyl)amino, 5-methyl-2,5-diazaspiro[3.4]oct-2-yl, (3aR,6aR)-5-methylhexa-hydro-pyrrolo[3,4-b]pyrrol-1(2H)-yl, 1-methyl-1,2,3,6-tetrahydropyridin-4-yl, 4-methylpiperizin-1-yl, 4-[2-(dimethylamino)-2-oxoethyl]piperazin-1-yl, methyl[2-(4-methylpiperazin-1-yl)ethyl]amino, methyl[2-(morpholin-4-yl)ethyl]amino, 1-amino-1,2,3,6-tetrahydropyridin-4-yl and 4-[(2S)-2-aminopropanoyl]piperazin-1-yl.

In another embodiment of formula (I), R³ is —N(R¹⁰)C₂₋₆ alkyl-NR¹⁰R¹⁰. In another embodiment, R³ is —N(R¹⁰)C₂₋₆ alkyl-NR¹⁰R¹⁰, wherein R¹⁰ is not H.

In another embodiment of formula (I), R¹ is selected from H, F, Cl, Br, CF₃, —CN, methyl, —CHF₂, ethynyl, methoxy, ethoxy, isopropoxy, —OCF₃, —OCH₂CF₃, —OCH₂CHF₂, —CHO, —CONH₂, —CONHMe, or —CONMe₂.

In another embodiment of formula (I), E³ is N.

In another embodiment of formula (I), E¹ and E² are each CH.

In one embodiment of formula (I), E¹, E² and E³, together with the nitrogen and carbon atoms of the six-member ring, form a heteroaromatic ring selected from the group consisting of

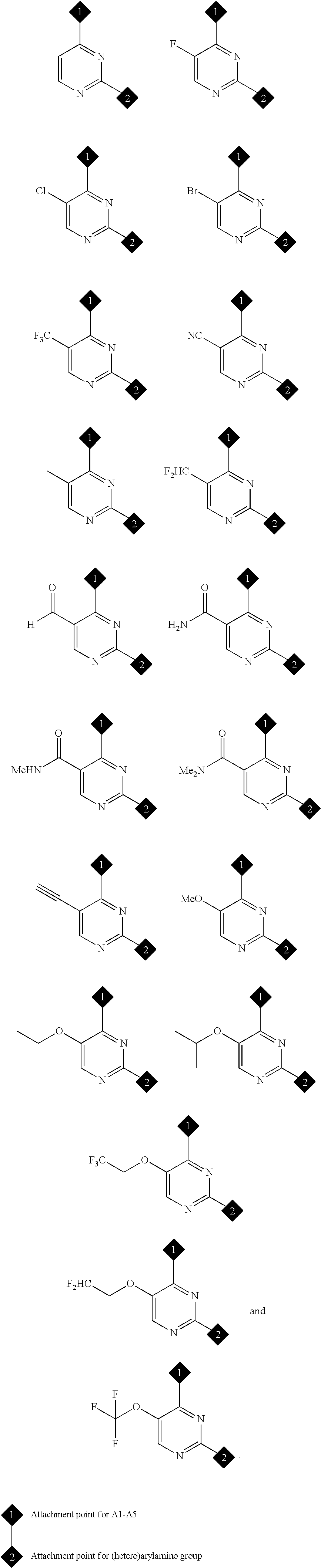

In another embodiment of formula (I), Y is

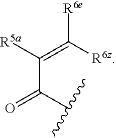

In one embodiment, the present invention relates to compounds of the formula (I), as disclosed herein, and compositions thereof. In one embodiment, the compounds of Formula (I) exclude the compounds exemplified in CN 105085489 A, WO 2015/127872, WO2013/014448, CN 105001208 A, CN 104844580 A, WO 2015/175632, WO 2015/188777, WO 2016/105525, WO2016060443, WO 2016/029839, WO 2016/054987, WO 2016/015453, WO 2016/070816, and/or WO 2015/195228. In one embodiment, the compounds of Formula (I) exclude the compounds exemplified in CN 104761585 A and/or CN 104761544 A.

Various embodiments disclosed herein for formula (I) can also be applied to formulae (A), (B), (C), (C-I), (D), (D-I), (E), (E-I), (F), (G), (H), (H-I), (J), (K), (L), (M), (N), (O), and/or (P) below.

In one embodiment, the compound of disclosure relates to a compound of formula (A) or (B):

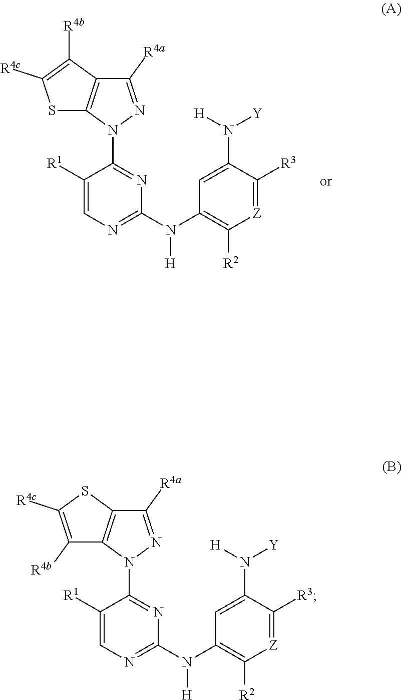

or a stereoisomer or a pharmaceutically acceptable salt, solvate, ester, or prodrug thereof;
wherein,
Z is CH or N;
Y is

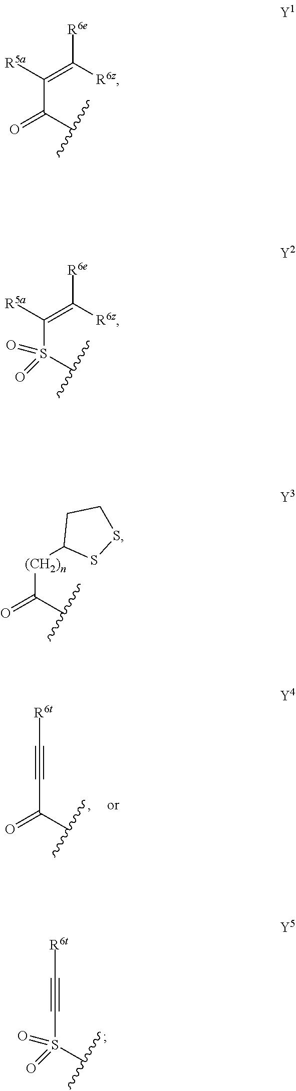

in Y¹ and Y², R^{5a} is H, F, Cl, CF₃, CHF₂, CF₂C₁₋₆ alkyl, CF₂CH₂NR⁸R⁹, CH₂NR⁸R⁹, CN, or C₁₋₆ alkyl;
in Y¹ and Y², R^{6e} is R¹⁰, H, F, aryl, heteroaryl, cycloalkyl, heterocycloalkyl, (CH₂)ₘCHR¹⁰R⁷, CF₂(CH₂)ₘCHR¹⁰R⁷, or C(R¹⁰)₂R⁷;
in Y⁴ and Y⁵, R^{6t} is C₁₋₆ alkyl, C₃₋₆ cycloalkyl, aryl, heteroaryl, heterocycloalkyl, (CH₂)ₘCHR¹⁰R⁷, C(R¹⁰)₂R⁷;
in Y¹ and Y², R^{6Z} is H, F, Cl, CF₃, CHF₂, CF₂C₁₋₆ alkyl or C₁₋₆ alkyl; or
alternatively in Y¹ and Y², R^{6e} and R^{6z}, taken together, form ═CR^{6e′}R^{6z′} (allene), wherein R^{6e′} is R¹⁰, H, F, aryl, heteroaryl, cycloalkyl, heterocycloalkyl, (CH₂)ₘCHR¹⁰R⁷, CF₂(CH₂)ₘCHR¹⁰R⁷, or C(R¹⁰)₂R⁷ and wherein, R^{6z′} is H, F, Cl, CF₃, CHF₂, CF₂C₁₋₆ alkyl or C₁₋₆ alkyl; or
alternatively in Y¹ and Y², R^{6e} and R^{6z}, taken together with the sp² carbon atom to which both are attached, form an alicyclic ring of 4 to 7 members wherein one of the ring atoms are optionally replaced by NR⁸, O, S(O)ₓ, S(═O)(═NR⁸), P═O, P(═O)(OR⁸), OP(═O)(OR⁸)O, and the alicyclic ring is optionally substituted with one or more substituents selected from the group consisting of halogen, oxo, OH, OR⁸, and NR⁸R⁹;
R¹ is independently selected from hydrogen, fluoro, chloro, bromo, methyl, ethyl, hydroxyl, methoxy, ethoxy, isopropoxy, cyclopropoxy, —OCF₃, —OCH₂CF₃, —OCH₂CHF₂, ethenyl, ethynyl, —CF₃, —CHF₂, —CHO, —CH₂OH, —CONH₂, —CO₂Me, —CONHMe, —CONMe₂, and cyano;
R² is —OCF₃, —OCHF₂, —OCF₂CF₃, —OCH₂CHF₂, —OCH₂CF₃, cyclopropyl, cyclopropoxy, methoxy, —OCD₃, ethoxy, or isopropoxy;
R³ is —N(R¹⁰)C₂₋₆ alkyl-NR¹⁰R¹⁰, —N(R¹⁰)C₂₋₆ alkyl-R⁷, —O(CH₂)ₚR⁷, —N(R¹⁰)C(═O)(CH₂)ₚR⁷, or R⁷;
each R^{4a}, R^{4b}, and R^{4c} are independently H, cyano, nitro, halo, —C₁₋₆ alkyl, —C₁₋₆ haloalkyl, -carboxy-C₁₋₆ alkyl, —C₁₋₆ hydroxyalkyl, R⁸R⁹N—C₁₋₆ alkyl-, —C₂₋₆ alkenyl, —C₂₋₆ alkynyl, C₁₋₆ acyl-, R—(CH₂)ₚC(═O)—, C₁₋₆ hydroxyalkyl-C(═O)—, carboxy, —C₁₋₆ alkoxycarbonyl, —C(═O)NR⁸R⁹, hydroxyl, —C₁₋₆ alkoxy, —C₁₋₆ acyloxy, —NR⁸R⁹, C₁₋₆ acyl-N(R¹⁰)—, pyrazole, 1,2,3-triazole, tetrazole, (C₁₋₆ alkyl)SO₂—, or R⁷SO₂—;
R⁷ is OH, NR⁸R⁹, O(CH₂)_{q}NR⁸R⁹, C₁₋₆ alkoxy, C₁₋₆ alkoxy-C₁₋₆ alkoxy, C₂₋₆ hydroxyalkoxy, oxetanyl, oxetanyloxy, oxetanylamino, oxolanyl, oxolanyloxy, oxolanylamino, oxanyl oxanyloxy, oxanylamino, oxepanyl, oxepanyloxy, oxepanylamino, azetidinyl, azetidinyloxy, azetidylamino, pyrrolidinyl, pyrolidinyloxy, pyrrolidinylamino, piperidinyl, piperidinyloxy, piperidinylamino, azepanyl, azepanyloxy, azepanylamino, dioxolanyl, dioxanyl, morpholino, thiomorpholino, thiomorpholino-S,S-dioxide, piperazino, dioxepanyl, dioxepanyloxy, dioxepanylamino, oxazepanyl, oxazepanyloxy, oxazepanylamino, diazepanyl, diazepanyloxy, diazepanylamino, (3R)-3-(dimethylamino)pyrrolidin-1-yl, (3S)-3-(dimethylamino)pyrrolidin-1-yl, 3-(dimethylamino)azetidin-1-yl, [2-(dimethylamino)ethyl](methyl)amino, [2-(methylamino)ethyl](methyl)amino, 5-methyl-2,5diazaspiro[3.4]oct-2-yl, (3aR,6aR)-5-methylhexa-hydro-pyrrolo[3,4-b]pyrrol-1(2H)-yl, I-methyl-1,2,3,6-tetrahydropyridin-4-yl, 4-methylpiperizin-1-yl, 4-[2(dimethylamino)-2-oxoethyl]piperazin-1-yl, methyl[2-(4-methylpiperazin-1yl)ethyl]amino, methyl[2-(morpholin-4-yl)ethyl]amino, 1-amino-1,2,3,6tetrahydropyridin-4-yl, 4-[(2S)-2-aminopropanoyl]piperazin-1-yl, all of which may be optionally substituted with OH, OR¹⁰, oxo, halogen, R¹⁰, CH₂OR¹⁰, or CH₂NR⁸R⁹;
R⁸ and R⁹ are each independently H, —CD₃, C₁₋₆ alkyl, C₃₋₆ alkenyl, C₃₋₆ alkynyl, C₃₋₈ cycloalkyl, —(C₁₋₃ alkyl)-(C₃₋₈ cycloalkyl), C₃₋₈ cycloalkenyl, C₁-C₆ acyl, 4-12 membered monocyclic or bicyclic heterocyclyl, 4-12 membered monocyclic or bicyclic heterocyclyl-C₁-C₆ alkyl-, C₆-C₁₂ aryl, 5-12 membered heteroaryl; wherein R⁸ and R⁹ may be further independently substituted with up to three substituents chosen from hydroxyl, C₁₋₆ alkoxy, C₁₋₆ hydroxyalkyl, C₁₋₆ alkoxy-C₁₋₆ alkyl, C₁₋₆ alkoxy-C₁₋₆ alkoxy, C₂₋₆ hydroxyalkoxy, oxo, thiono, cyano or halo; or
alternatively, R⁸ and R⁹, taken together with the N atom to which they are both attached, form a heterocyclic ring of 4-7 members, containing up to one other heteroatom selected from O, S, or NR¹¹, or a heterobicyclic ring of 7-12 members which may be fused, bridged or spiro, and contain up to two other heteroatoms chosen from O, S(O)ₓ, or NR¹¹, and these heterocyclic rings are optionally substituted with up to three substituents chosen from hydroxyl, C₁₋₆ alkoxy, C₁₋₆ hydroxyalkyl, C₁₋₆ alkoxy-C₁₋₆ alkyl, C₁₋₆ alkoxy-C₁₋₆ alkoxy, C₂₋₆ hydroxyalkoxy, oxo, thiono, cyano or halo;
each R¹⁰ is independently H, —CD₃, C₁₋₆ alkyl, C₃₋₆ cycloalkyl, C₂₋₆ hydroxyalkyl, C₁₋₆ alkoxy-C₁₋₆ alkyl or C₂₋₆ alkyl-NR⁸R⁹;
alternatively, two R¹⁰ on the same N atom to which they are both attached, form a heterocyclic ring of 5-6 members, containing up to one other heteroatom selected from O, S, or NR¹¹;
each R¹¹ is independently hydrogen or C₁-C₆ alkyl, which is optionally substituted with up to three substituents selected from hydroxyl, oxo, thiono, cyano or halo;
n is 1, 2, or 3;
q is 2, 3, or 4;
p is 0, 1, 2, 3, or 4; and
x is 0, 1, or 2.

In another embodiment, the present disclosure relates to a compound having the structure of formula (A):

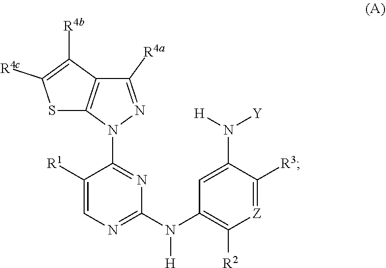

or a stereoisomer or a pharmaceutically acceptable salt, solvate, ester, or prodrug thereof, wherein,
Z is CH or N;
R¹ is selected from hydrogen, fluoro, chloro, bromo, methyl, CF₃, CHF₂, and cyano;
R² is —OCF₃, —OCHF₂, —OCF₂CF₃, —OCH₂CHF₂, —OCH₂CF₃, cyclopropyl, cyclopropoxy, methoxy, —OCD₃, ethoxy, or isopropoxy;
R³ is N(R¹⁰)C₂₋₆ alkyl-NR¹⁰R¹⁰;
R^{4a}, R^{4b} and R^{4c} are each independently H, cyano, halo, —C₁₋₆ alkyl, —C₁₋₆ haloalkyl, carboxy-C₁₋₆ alkyl, —C₁₋₆ hydroxyalkyl, R⁸R⁹N—C₁₋₆ alkyl-, —C₂₋₆ alkenyl, —C₂₋₆ alkynyl, C₁₋₆ acyl-, R—(CH₂)ₚC(═O)—, C₁₋₆ hydroxyalkyl-C(═O)—, carboxy, —C₁₋₆ alkoxycarbonyl, —C(═O)NR⁸R⁹, hydroxyl, alkoxy, C₁₋₆ acyloxy, —NR⁸R⁹, C₁₋₆ acyl-N(R¹⁰)—, R⁷SO₂—,
R⁷ is OH, NR⁸R⁹, O(CH₂)_{q}NR⁸R⁹, C₁₋₆ alkoxy, or C₂₋₆ hydroxyalkoxy;
R⁸ and R⁹ are independently H, —CD₃, C₁₋₆ alkyl, C₃₋₆ alkenyl, C₃₋₆ alkynyl, C₃₋₈ cycloalkyl, C₃₋₈ cycloalkenyl, C₁-C₆ acyl, 4-12 membered monocyclic or bicyclic heterocyclyl, 4-12 membered monocyclic or bicyclic heterocyclyl-C₁-C₆ alkyl-, C₆-C₁₂ aryl, 5-12 membered heteroaryl; and R⁸ and R⁹ may be further independently substituted with up to three substituents chosen from hydroxyl, C₁₋₆ alkoxy, C₁₋₆ hydroxyalkylC₂₋₆ hydroxyalkoxy, oxo, thiono, cyano or halo; or
alternatively, R⁸ and R⁹, taken together with the N atom to which they are both attached, form a heterocyclic ring of 4-7 members, containing up to one other heteroatom selected from O, S, or NR¹¹, or a heterobicyclic ring of 7-12 members which may be fused, bridged or spiro, and contain up to two other heteroatoms chosen from O, S(O)ₓ, or NR¹¹, and these heterocyclic rings are optionally substituted with up to three substituents chosen from hydroxyl, C₁₋₆ alkoxy, C₁₋₆ hydroxyalkyl, C₁₋₆ alkoxy-C₁₋₆ alkyl, C₁₋₆ alkoxy-C₁₋₆ alkoxy, C₂₋₆ hydroxyalkoxy, oxo, thiono, cyano or halo;
each R¹⁰ is independently H, —CD₃, C₁₋₆ alkyl, C₃₋₆ cycloalkyl, C₂₋₆ hydroxyalkyl, C₁₋₆ alkoxy-C₁₋₆ alkyl or C₂₋₆ alkyl-NR⁸R⁹; or
p is 0, 1, 2, 3, or 4;
q is 2, 3, or 4; and
x is 0, 1, or 2.

In some embodiments, R³ in formula (A) or (B) is —N(CH₃)CH₂CH₂NR¹⁰R¹⁰. In other embodiments, R³ in formula (A) or (B) is —N(CH₃)CH₂CH₂NR¹⁰R¹⁰, wherein each R¹⁰ is independently —CD₃, C₁₋₆ alkyl, C₃₋₆ cycloalkyl, C₂₋₆ hydroxyalkyl, C₁₋₆ alkoxy-C₁₋₆ alkyl or C₂₋₆ alkyl-NR⁸R⁹.

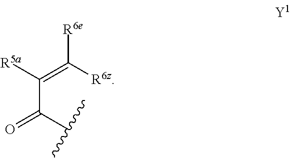

In one embodiment, Y in formula (A) or (B) is. In some embodiments, R^{5a}, R^{6e}, and R^{6z} are each independently H.

In one embodiment, the compound of the present disclosure has the structure of formula (C):

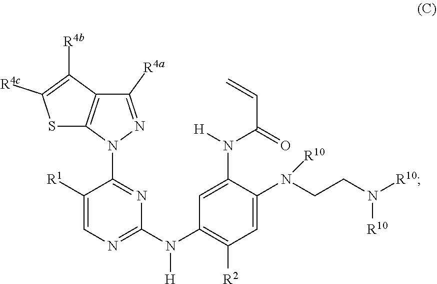

or a stereoisomer or a pharmaceutically acceptable salt, solvate, ester, or prodrug thereof;
wherein,
R¹ is hydrogen, fluoro, chloro, or methyl;
R² is —OCF₃, —OCHF₂, —OCF₂CF₃, —OCH₂CHF₂, —OCH₂CF₃, cyclopropyl, cyclopropoxy, methoxy, —OCD₃, ethoxy, or isopropoxy;
R^{4a} is H or —NR⁸R⁹;
R^{4b} and R^{4c} are each independently H, cyano, F, Cl, Br, CH₃, CF₃, CHF₂, CONH₂, or C(═O)NR⁸R⁹;
R⁸ and R⁹ are each independently H, —CD₃, or C₁₋₆ alkyl; and
each R¹⁰ is independently H, —CD₃, C₁₋₆ alkyl, C₃₋₆ cycloalkyl, or C₂₋₆ hydroxyalkyl.

In one embodiment, R^{4b} and R^{4c} are each independently H, cyano, F, Cl, Br, CH₃, CF₃, or CHF₂.

In another embodiment, the compound of formula (C) comprises:
R¹ is hydrogen;
R² is methoxy, —OCD₃, ethoxy, or isopropoxy;
R^{4a} is NR⁸R⁹;
R^{4b} is H, or CH₃;
R^{4c} is H, F, Cl, Br, or CH₃;
R⁸ and R⁹ are each independently H, —CD₃, —CH₃, —CH₂CH₃, or —CH(CH₃)₂; and
each R¹⁰ is independently H, —CD₃, —CH₃, —CH₂CH₃, or —CH(CH₃)₂.
   In one embodiment, the compound of the present disclosure has the structure of (C-I):

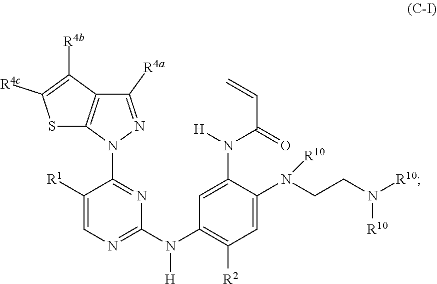

or a stereoisomer or a pharmaceutically acceptable salt, solvate, ester, or prodrug thereof;
wherein,
R¹ is hydrogen, fluoro, chloro, or methyl;
R² is —OCF₃, —OCHF₂, —OCF₂CF₃, —OCH₂CHF₂, —OCH₂CF₃, cyclopropyl, cyclopropoxy, methoxy, —OCD₃, ethoxy, or isopropoxy;
R^{4a} is H or —NR⁸R⁹;
R^{4b} and R^{4c} are each independently H, cyano, F, Cl, Br, —C₁₋₆ alkyl, —CF₃, —CHF₂, —CONH₂, or —C(═O)NR⁸R⁹;
R⁸ and R⁹ are each independently H, —CD₃, or —C₁₋₆ alkyl; and
each R¹⁰ is independently H, —CD₃, —C₁₋₆ alkyl, —C₃₋₆ cycloalkyl, or —C₂₋₆ hydroxyalkyl.

In another embodiment, the compound of formula (C-I) comprises:
R¹ is hydrogen;
R² is methoxy, —OCD₃, ethoxy, or isopropoxy;
R^{4a} is NR⁸R⁹;
R^{4b} is H, or CH₃;
R^{4c} is H, F, Cl, Br, —CF₃, —CH₃, or —CH₂CH₃;
R⁸ and R⁹ are each independently H, —CD₃, —CH₃, —CH₂CH₃, or —CH(CH₃)₂; and
each R¹⁰ is independently H, —CD₃, —CH₃, —CH₂CH₃, or —CH(CH₃)₂.

In one embodiment, R¹⁰ in formula (C) or (C-I) is each —CD₃, C₁₋₆ alkyl, C₃₋₆ cycloalkyl, or C₂₋₆ hydroxyalkyl.

In another embodiment, R¹⁰ in formula (A), (B), (C) and/or (C-I) is each independently H, —CD₃, C₁₋₆ alkyl, C₃₋₆ cycloalkyl, or C₂₋₆ hydroxyalkyl. In other embodiments, R¹⁰ is each independently H, —CD₃, methyl, ethyl, or isopropyl.

In another embodiment, R¹⁰ in formula (A), (B), (C) and/or (C-I) is each independently —CD₃, C₁₋₆ alkyl, C₃₋₆ cycloalkyl, or C₂₋₆ hydroxyalkyl. In other embodiments, R¹⁰ is each independently —CD₃, methyl, ethyl, or isopropyl.

In another embodiment, R^{4a} in formula (A), (B), (C) and/or (C-I) is each independently H, —C₁₋₆ alkyl, or —NR⁸R⁹. In one embodiment, R^{4a} is —NR⁸R⁹. In one embodiment, R⁸ and R⁹ are independently H, —CD₃, or C₁₋₆ alkyl. In another embodiment, R^{4a} is —N(CH₃)₂.

In some embodiments, R^{4b} and R^{4c} in formula (A), (B), (C) and/or (C-I) are each independently H, cyano, F, Cl, Br, CH₃, CF₃, CHF₂, C(═O)NR⁸R⁹, or CONH₂. In other embodiments, R^{4b} and R^{4c} in formula (A), (B), (C) and/or (C-I) are each independently H, cyano, F, Cl, Br, CH₃, CF₃, or CHF₂. In one embodiment, R^{4b} is H. In other embodiments, R^{4c} is H, F, Cl, or Br. In some embodiments, R^{4c} is H or Cl.

In one embodiment, the compound of the present disclosure has the structure of formula (D):

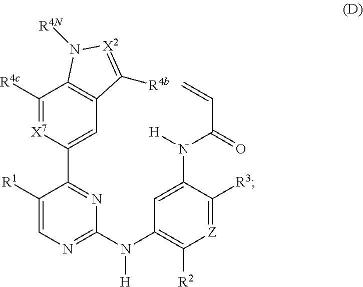

or a stereoisomer or a pharmaceutically acceptable salt, solvate, ester, or prodrug thereof;
wherein,
Z is CH or N;
X² and X⁷ are each CH, CR⁴, or N;
R¹ is hydrogen, fluoro, chloro, bromo, methyl, ethyl, hydroxyl, methoxy, ethoxy, isopropoxy, cyclopropoxy, —OCF₃, —OCH₂CF₃, —OCH₂CHF₂, ethenyl, ethynyl, CF₃, CHF₂, CHO, CH₂OH, CONH₂, CO₂Me, CONHMe, CONMe₂, or cyano;
R² is —OCF₃, —OCHF₂, —OCF₂CF₃, —OCH₂CHF₂, —OCH₂CF₃, cyclopropyl, cyclopropoxy, methoxy, —OCD₃, ethoxy, or isopropoxy;
R³ is N(R¹⁰)C₂₋₆ alkyl-NR¹⁰R¹⁰;
each R⁴ is independently H, cyano, halo, —C₁₋₆ alkyl, —C₁₋₆ haloalkyl, carboxy-C₁₋₆ alkyl, —C₁₋₆ hydroxyalkyl, R⁸R⁹N—C₁₋₆ alkyl-, —C₂₋₆ alkenyl, —C₂₋₆ alkynyl, C₁₋₆ acyl-, R⁷—(CH₂)ₚC(═O)—, C₁₋₆ hydroxyalkyl-C(═O)—, carboxy, —C₁₋₆ alkoxycarbonyl, —C(═O)NR⁸R⁹, hydroxyl, alkoxy, C₁₋₆ acyloxy, —NR⁸R⁹, C₁₋₆ acyl-N(R¹⁰)—, or R⁷SO₂—; and
R⁸ and R⁹ are independently H, —CD₃, C₁₋₆ alkyl, C₃₋₆ alkenyl, C₃₋₆ alkynyl, C₃₋₈ cycloalkyl, C₃₋₈ cycloalkenyl, C₁-C₆ acyl, 4-12 membered monocyclic or bicyclic heterocyclyl, 4-12 membered monocyclic or bicyclic heterocyclyl-C₁-C₆ alkyl-, C₆-C₁₂ aryl, 5-12 membered heteroaryl; and R⁸ and R⁹ may be further independently substituted with up to three substituents chosen from hydroxyl, C₁₋₆ alkoxy, C₁₋₆ hydroxyalkylC₂₋₆ hydroxyalkoxy, oxo, thiono, cyano or halo; or
alternatively, R⁸ and R⁹, taken together with the N atom to which they are both attached, form a heterocyclic ring of 4-7 members, containing up to one other heteroatom selected from O, S, or NR¹¹, or a heterobicyclic ring of 7-12 members which may be fused, bridged or spiro, and contain up to two other heteroatoms chosen from O, S(O)ₓ, or NR¹¹, and these heterocyclic rings are optionally substituted with up to three substituents chosen from hydroxyl, C₁₋₆ alkoxy, C₁₋₆ hydroxyalkyl, C₁₋₆ alkoxy-C₁₋₆ alkyl, C₁₋₆ alkoxy-C₁₋₆ alkoxy, C₂₋₆ hydroxyalkoxy, oxo, thiono, cyano or halo;
R^{4b} is H, halo, —C₁₋₆ alkyl, or —C₁₋₆ haloalkyl;
R^{4c} is cyano, C₁₋₆ acyl-, —C(═O)NR⁸R⁹, hydroxyl, alkoxy, or F;
R^{4N} is H, —CD₃, or —C₁₋₆ alkyl;
R⁷ is OH, NR⁸R⁹, —O(CH₂)_{q}NR⁸R⁹, C₁₋₆ alkoxy, or C₂₋₆ hydroxyalkoxy;
each R¹⁰ is independently H, —CD₃, C₁₋₆ alkyl, C₃₋₆ cycloalkyl, C₂₋₆ hydroxyalkyl, C₁₋₆ alkoxy-C₁₋₆ alkyl or C₂₋₆ alkyl-NR⁸R⁹;
p=0, 1, 2, 3, or 4;
q=2, 3, or 4; and
x=0, 1, or 2.

In one embodiment, each R¹⁰ in formula (D) is independently —CD₃, C₁₋₆ alkyl, C₃₋₆ cycloalkyl, C₂₋₆ hydroxyalkyl, C₁₋₆ alkoxy-C₁₋₆ alkyl or C₂₋₆ alkyl-NR⁸R⁹.

In one embodiment, the present disclosure relates to compounds of formula (D-I):

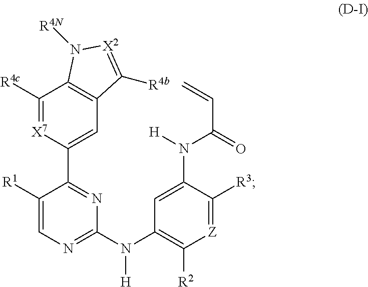

or a stereoisomer or a pharmaceutically acceptable salt, solvate, N-oxide, ester, or prodrug thereof;
wherein,
Z is CH or N;
X² and X⁷ are each CH, CR⁴, or N;
R¹ is hydrogen, fluoro, chloro, bromo, methyl, ethyl, hydroxyl, methoxy, ethoxy, isopropoxy, cyclopropoxy, —OCF₃, —OCH₂CF₃, —OCH₂CHF₂, ethenyl, ethynyl, CF₃, CHF₂, CHO, CH₂OH, CONH₂, CO₂Me, CONHMe, CONMe₂, or cyano;
R² is —OCF₃, —OCHF₂, —OCF₂CF₃, —OCH₂CHF₂, —OCH₂CF₃, cyclopropyl, cyclopropoxy, methoxy, —OCD₃, ethoxy, or isopropoxy;
R³ is —N(R¹⁰)(C₂₋₆ alkyl)-NR¹⁰R¹⁰ or —N(R¹⁰)(C₃₋₁₀ cycloalkylalkyl)-NR¹⁰R¹⁰;
each R⁴ is independently H, cyano, halo, —C₁₋₆ alkyl, —C₁₋₆ haloalkyl, carboxy-C₁₋₆ alkyl, —C₁₋₆ hydroxyalkyl, R⁸R⁹N—C₁₋₆ alkyl-, —C₂₋₆ alkenyl, —C₂₋₆ alkynyl, C₁₋₆ acyl-, R⁷—(CH₂)ₚC(═O)—, C₁₋₆ hydroxyalkyl-C(═O)—, carboxy, —C₁₋₆ alkoxycarbonyl, —C(═O)NR⁸R⁹, hydroxyl, alkoxy, C₁₋₆ acyloxy, —NR⁸R⁹, C₁₋₆ acyl-N(R¹⁰)—, or R⁷SO₂—; and
R⁸ and R⁹ are independently H, —CD₃, C₁₋₆ alkyl, C₃₋₆ alkenyl, C₃₋₆ alkynyl, C₃₋₈ cycloalkyl, C₃₋₈ cycloalkenyl, C₁-C₆ acyl, 4-12 membered monocyclic or bicyclic heterocyclyl, 4-12 membered monocyclic or bicyclic heterocyclyl-C₁-C₆ alkyl-, C₆-C₁₂ aryl, 5-12 membered heteroaryl; and R⁸ and R⁹ may be further independently substituted with up to three substituents chosen from hydroxyl, C₁₋₆ alkoxy, C₁₋₆ hydroxyalkylC₂₋₆ hydroxyalkoxy, oxo, thiono, cyano or halo; or
alternatively, R⁸ and R⁹, taken together with the N atom to which they are both attached, form a heterocyclic ring of 4-7 members, containing up to one other heteroatom selected from O, S, or NR¹¹, or a heterobicyclic ring of 7-12 members which may be fused, bridged or spiro, and contain up to two other heteroatoms chosen from O, S(O)ₓ, or NR¹¹, and these heterocyclic rings are optionally substituted with up to three substituents chosen from hydroxyl, C₁₋₆ alkoxy, C₁₋₆ hydroxyalkyl, C₁₋₆ alkoxy-C₁₋₆ alkyl, C₁₋₆ alkoxy-C₁₋₆ alkoxy, C₂₋₆ hydroxyalkoxy, oxo, thiono, cyano or halo;
R^{4b} is H, halo, —C₁₋₆ alkyl, or —C₁₋₆ haloalkyl;
R^{4c} is H, cyano, hydroxyl, alkoxy, —C₁₋₆ alkyl, or —C₁₋₆ haloalkyl, Cl, or F, provided that when R^{4c} is H, R^{4b} is halo, —C₁₋₆ alkyl, or —C₁₋₆ haloalkyl;
R^{4N} is H, —CD₃, or —C₁₋₆ alkyl;
R⁷ is OH, NR⁸R⁹, —O(CH₂)_{q}NR⁸R⁹, C₁₋₆ alkoxy, or C₂₋₆ hydroxyalkoxy;
each R¹⁰ is independently H, —CD₃, C₁₋₆ alkyl, C₃₋₆ cycloalkyl, C₂₋₆ hydroxyalkyl, C₁₋₆ alkoxy-C₁₋₆ alkyl or C₂₋₆ alkyl-NR⁸R⁹; or
alternatively, two R¹⁰ on the same N atom, taken together form a heterocyclic ring of 3-7 members, optionally substituted with up to three substituents chosen from hydroxyl, C₁₋₆ alkoxy, C₁₋₆ hydroxyalkyl, C₁₋₆ alkoxy-C₁₋₆ alkyl, C₁₋₆ alkoxy-C₁₋₆ alkoxy, C₂₋₆ hydroxyalkoxy, oxo, thiono, cyano or halo;
p=0, 1, 2, 3, or 4;
q=2, 3, or 4; and
x=0, 1, or 2.

In one embodiment of the compound of formula (D-I),
X² is CH or CR⁴;
R⁴ is methyl, ethyl, or isopropyl;
R^{4c} is cyano, —CF₃, Cl, or F;
R^{4N} is —CD₃, methyl, ethyl, or isopropyl; and
R^{4b} is H, halo, methyl, ethyl, or isopropyl.

In one embodiment of the compound of formula (D-I), R³ is —N(R¹⁰)(C₃₋₁₀ cycloalkylalkyl)-NR¹⁰R¹⁰, wherein C₃₋₁₀ cycloalkylalkyl is selected from:

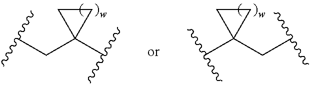

where w is 1, 2, 3, 4, or 5. In one embodiment of the compound of formula (D-I), R³ is —N(R¹⁰)(C₂₋₆ alkyl)-NR¹⁰R¹⁰, wherein two R¹⁰ on the same N atom, taken together form a heterocyclic ring of 3-7 members, optionally substituted with up to three substituents chosen from hydroxyl, C₁₋₆ alkoxy, C₁₋₆ hydroxyalkyl, C₁₋₆ alkoxy-C₁₋₆ alkyl, C₁₋₆ alkoxy-C₁₋₆ alkoxy, C₂₋₆ hydroxyalkoxy, oxo, thiono, cyano or halo. In one embodiment of the compound of formula (D-I), R³ is

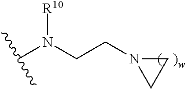

where w is 1, 2, 3, 4, or 5. In one embodiment of the compound of formula (D-I), R³ is

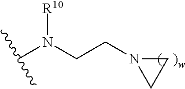

where w is 1, 2, 3, 4, or 5 and R¹⁰ is H, —CD₃, methyl, ethyl, propyl, or isopropyl. In one embodiment of the compound of formula (D-I), R³ is —N(R¹⁰)(C₂₋₆ alkyl)-NR¹⁰R¹⁰, wherein C₂₋₆ alkyl is linear or branched. In one embodiment of the compound of formula (D-I), R³ is —N(R¹⁰)(C₂₋₆ alkyl)-NR¹⁰R¹⁰, wherein C₂₋₆ alkyl is branched.

In one embodiment of the compound of formula (D-I), R¹⁰ is H, —CD₃, methyl, ethyl, propyl, or isopropyl.

In one embodiment of the compound of formula (D-I),
X² is N;
R^{4c} is cyano, —CF₃, Cl, or F;
R^{4N} is —CD₃, methyl, ethyl, or isopropyl; and
R^{4b} is H, halo, methyl, ethyl, or isopropyl.

In one embodiment of the compound of formula (D-I), X² is N and X⁷ is CH.

In one embodiment, the compound of the present disclosure has the structure of formula (E):

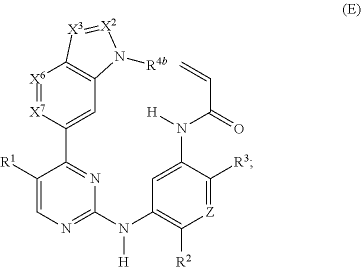

or a stereoisomer or a pharmaceutically acceptable salt, solvate, ester, or prodrug thereof;
wherein,
Z is CH or N;
X², X³, X⁶ and X⁷ are each CH, CR⁴, or N;
R¹ is hydrogen, fluoro, chloro, bromo, methyl, ethyl, hydroxyl, methoxy, ethoxy, isopropoxy, cyclopropoxy, —OCF₃, —OCH₂CF₃, —OCH₂CHF₂, ethenyl, ethynyl, CF₃, CHF₂, CHO, CH₂OH, CONH₂, CO₂Me, CONHMe, CONMe₂, or cyano;
R² is —OCF₃, —OCHF₂, —OCF₂CF₃, —OCH₂CHF₂, —OCH₂CF₃, cyclopropyl, cyclopropoxy, methoxy, —OCD₃, ethoxy, or isopropoxy;
R³ is N(R¹⁰)C₂₋₆ alkyl-NR¹⁰R¹⁰;
each R⁴ is independently H, cyano, halo, —C₁₋₆ alkyl, —C₁₋₆ haloalkyl, carboxy-C₁₋₆ alkyl, —C₁₋₆ hydroxyalkyl, R⁸R⁹N—C₁₋₆ alkyl-, —C₂₋₆ alkenyl, —C₂₋₆ alkynyl, C₁₋₆ acyl-, R⁷—(CH₂)ₚC(═O)—, C₁₋₆ hydroxyalkyl-C(═O)—, carboxy, —C₁₋₆ alkoxycarbonyl, —C(═O)NR⁸R⁹, hydroxyl, alkoxy, C₁₋₆ acyloxy, —NR⁸R⁹, C₁₋₆ acyl-N(R¹⁰)—, or R⁷SO₂—; and
R⁸ and R⁹ are independently H, —CD₃, C₁₋₆ alkyl, C₃₋₆ alkenyl, C₃₋₆ alkynyl, C₃₋₈ cycloalkyl, C₃₋₈ cycloalkenyl, C₁-C₆ acyl, 4-12 membered monocyclic or bicyclic heterocyclyl, 4-12 membered monocyclic or bicyclic heterocyclyl-C₁-C₆ alkyl-, C₆-C₁₂ aryl, 5-12 membered heteroaryl; and R⁸ and R⁹ may be further independently substituted with up to three substituents chosen from hydroxyl, C₁₋₆ alkoxy, C₁₋₆ hydroxyalkylC₂₋₆ hydroxyalkoxy, oxo, thiono, cyano or halo; or
alternatively, R⁸ and R⁹, taken together with the N atom to which they are both attached, form a heterocyclic ring of 4-7 members, containing up to one other heteroatom selected from O, S, or NR¹¹, or a heterobicyclic ring of 7-12 members which may be fused, bridged or spiro, and contain up to two other heteroatoms chosen from O, S(O)ₓ, or NR¹¹, and these heterocyclic rings are optionally substituted with up to three substituents chosen from hydroxyl, C₁₋₆ alkoxy, C₁₋₆ hydroxyalkyl, C₁₋₆ alkoxy-C₁₋₆ alkyl, C₁₋₆ alkoxy-C₁₋₆ alkoxy, C₂₋₆ hydroxyalkoxy, oxo, thiono, cyano or halo;
R^{4N} is H, —CD₃, or —C₁₋₆ alkyl;
R⁷ is OH, NR⁸R⁹, —O(CH₂)_{q}NR⁸R⁹, C₁₋₆ alkoxy, or C₂₋₆ hydroxyalkoxy;
each R¹⁰ is independently H, —CD₃, C₁₋₆ alkyl, C₃₋₆ cycloalkyl, C₂₋₆ hydroxyalkyl, C₁₋₆ alkoxy-C₁₋₆ alkyl or C₂₋₆ alkyl-NR⁸R⁹;
p=0, 1, 2, 3, or 4;
q=2, 3, or 4; and
x=0, 1, or 2.

In one embodiment, R¹⁰ in formula (E) is independently —CD₃, C₁₋₆ alkyl, C₃₋₆ cycloalkyl, C₂₋₆ hydroxyalkyl, C₁₋₆ alkoxy-C₁₋₆ alkyl or C₂₋₆ alkyl-NR⁸R⁹.

In one embodiment, the compound of the present disclosure has the structure of formula (F) or (G):

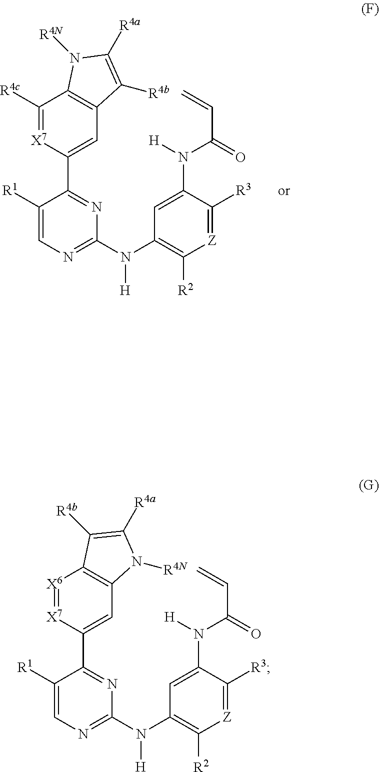

or a stereoisomer or a pharmaceutically acceptable salt, solvate, ester, or prodrug thereof;
wherein,
Z is CH or N;
X⁶ and X⁷ are each CH, CR⁴, or N;
R¹ is independently selected from hydrogen, fluoro, chloro, bromo, methyl, ethyl, hydroxyl, methoxy, ethoxy, isopropoxy, cyclopropoxy, —OCF₃, —OCH₂CF₃, —OCH₂CHF₂, ethenyl, ethynyl, CF₃, CHF₂, CHO, CH₂OH, CONH₂, CO₂Me, CONHMe, CONMe₂, and cyano;
R² is —OCF₃, —OCHF₂, —OCF₂CF₃, —OCH₂CHF₂, —OCH₂CF₃, cyclopropyl, cyclopropoxy, methoxy, —OCD₃, ethoxy, or isopropoxy;
R³ is N(R¹⁰)C₂₋₆ alkyl-NR¹⁰R¹⁰;
each R⁴ is independently H, cyano, halo, —C₁₋₆ alkyl, —C₁₋₆ haloalkyl, carboxy-C₁₋₆ alkyl, —C₁₋₆ hydroxyalkyl, R⁸R⁹N—C₁₋₆ alkyl-, —C₂₋₆ alkenyl, —C₂₋₆ alkynyl, C₁₋₆ acyl-, R⁷—(CH₂)ₚC(═O)—, C₁₋₆ hydroxyalkyl-C(═O)—, carboxy, —C₁₋₆ alkoxycarbonyl, —C(═O)NR⁸R⁹, hydroxyl, alkoxy, C₁₋₆ acyloxy, —NR⁸R⁹, C₁₋₆ acyl-N(R¹⁰)—, R⁷SO₂—,
R^{4a} and R^{4b} are each independently H, halo, —C₁₋₆ alkyl, or —C₁₋₆ haloalkyl;
R^{4c} is cyano, C₁₋₆ acyl-, —C(═O)NR⁸R⁹, hydroxyl, alkoxy, or F;
R^{4N} is H, —CD₃, —C₁₋₆ alkyl, or —C₁₋₆ haloalkyl;
R⁷ is OH, NR⁸R⁹, O(CH₂)_{q}NR⁸R⁹, C₁₋₆ alkoxy, or C₂₋₆ hydroxyalkoxy;
R⁸ and R⁹ are independently H, —CD₃, C₁₋₆ alkyl, C₃₋₆ alkenyl, C₃₋₆ alkynyl, C₃₋₈ cycloalkyl, C₃₋₈ cycloalkenyl, C₁-C₆ acyl, 4-12 membered monocyclic or bicyclic heterocyclyl, 4-12 membered monocyclic or bicyclic heterocyclyl-C₁-C₆ alkyl-, C₆-C₁₂ aryl, 5-12 membered heteroaryl; and R⁸ and R⁹ may be further independently substituted with up to three substituents chosen from hydroxyl, C₁₋₆ alkoxy, C₁₋₆ hydroxyalkylC₂₋₆ hydroxyalkoxy, oxo, thiono, cyano or halo; or
each R¹⁰ is independently H, —CD₃, C₁₋₆ alkyl, C₃₋₆ cycloalkyl, C₂₋₆ hydroxyalkyl, C₁₋₆ alkoxy-C₁₋₆ alkyl or C₂₋₆ alkyl-NR⁸R⁹; or
p=0, 1, 2, 3, or 4; and
q=2, 3, or 4.

In one embodiment, each R¹⁰ in formula (F) and/or (G) is independently —CD₃, C₁₋₆ alkyl, C₃₋₆ cycloalkyl, C₂₋₆ hydroxyalkyl, C₁₋₆ alkoxy-C₁₋₆ alkyl or C₂₋₆ alkyl-NR⁸R⁹.

In one embodiment, R³ in formula (D), (D-I), (E), (E-I), (F), and/or (G) is N(R¹⁰)C₂₋₆ alkyl-NR¹⁰R¹⁰. In one embodiment, R³ is —N(CH₃)CH₂CH₂NR¹⁰R¹⁰.

In another embodiment, R¹⁰ in formula (D), (D-I), (E), (E-I), (F), and/or (G) is each independently H, —CD₃, C₁₋₆ alkyl, C₃₋₆ cycloalkyl, or C₂₋₆ hydroxyalkyl. In other embodiments, R¹⁰ is each independently H, —CD₃, methyl, ethyl, or isopropyl.

In another embodiment, R¹⁰ in formula (D), (D-I), (E), (E-I), (F), and/or (G) is each independently —CD₃, C₁₋₆ alkyl, C₃₋₆ cycloalkyl, or C₂₋₆ hydroxyalkyl. In other embodiments, R¹⁰ is each independently —CD₃, methyl, ethyl, or isopropyl.

In one embodiment, R¹ in formula (D), (D-I), (E), (E-I), (F), and/or (G) is hydrogen, methyl, fluoro, chloro, bromo, CF₃, or cyano. In another embodiment, R¹ is H.

In one embodiment, R^{4c} in formula (D), (D-I), and/or (F), is —CN.

In one embodiment, the compound of formula (D), (D-I), (E), (E-I), (F), and/or (G) is not

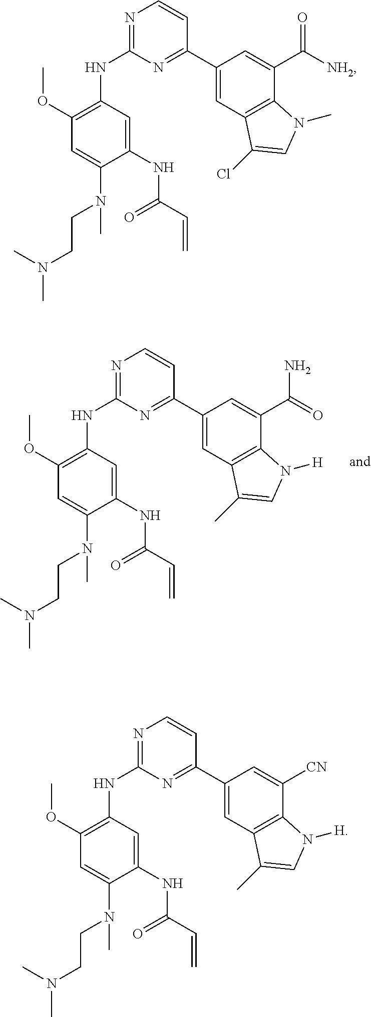

In another embodiment, the compound of formula (D), (D-I), (E), (E-I), (F), and/or (G) is

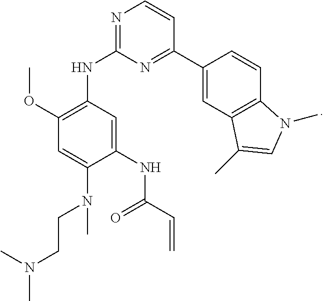

In another embodiment, the compound of formula (D), (D-I), (E), (E-I), (F), and/or (G) is

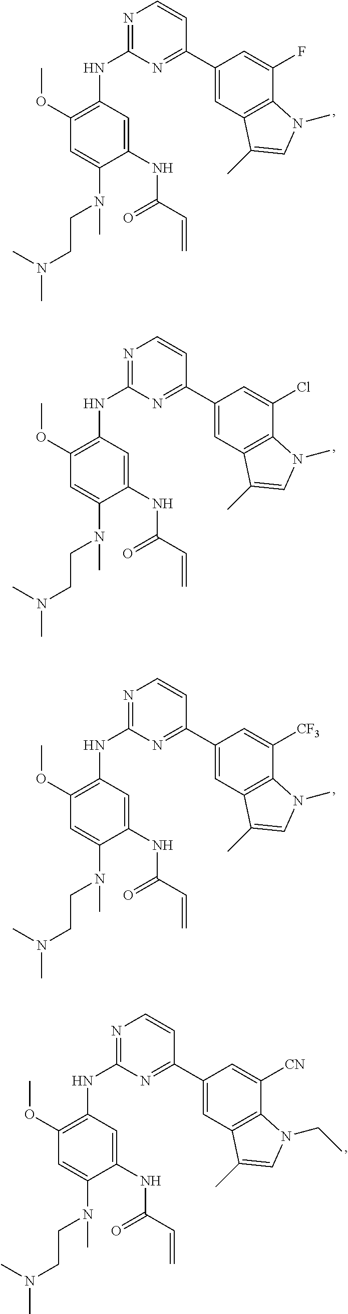

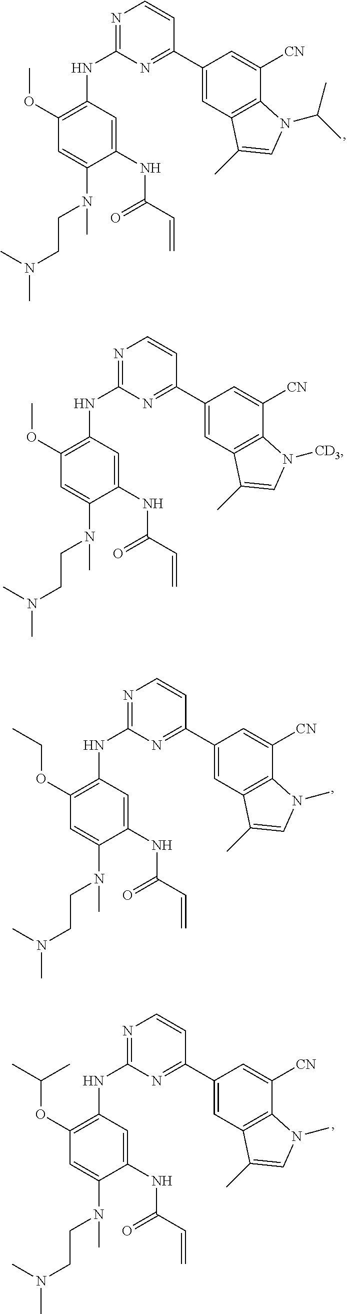

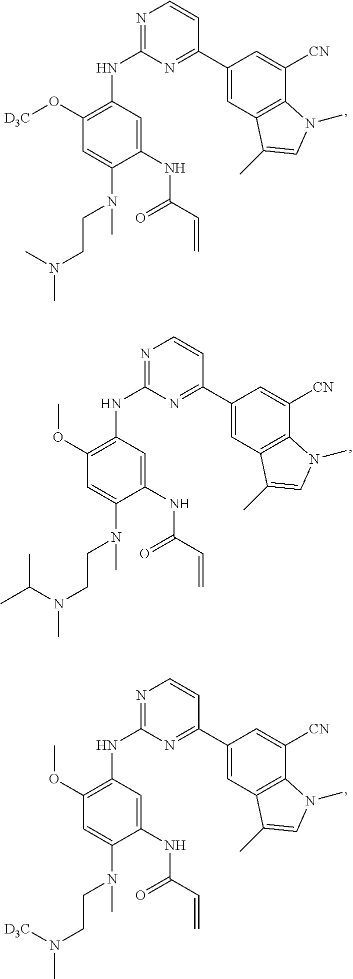

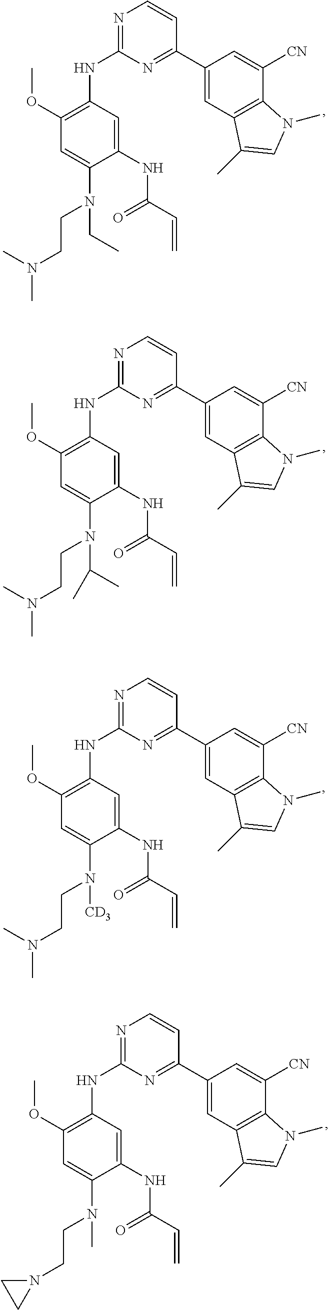

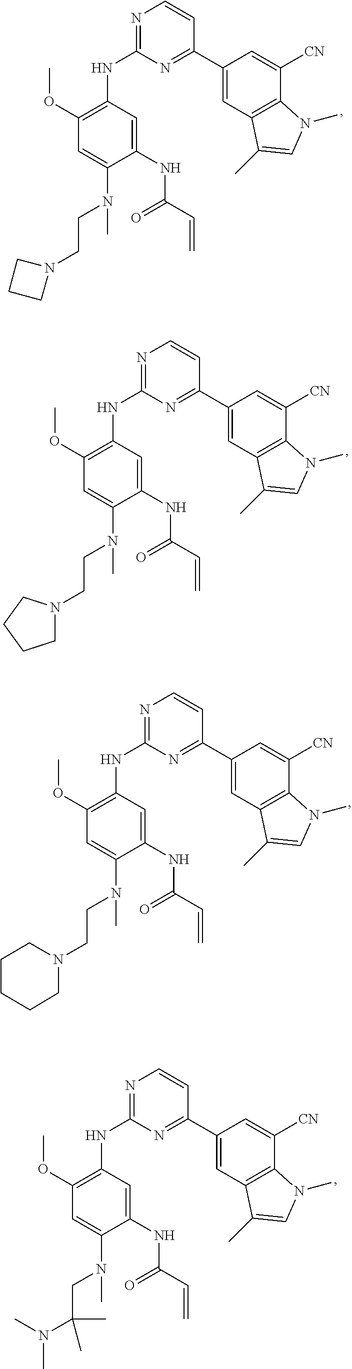

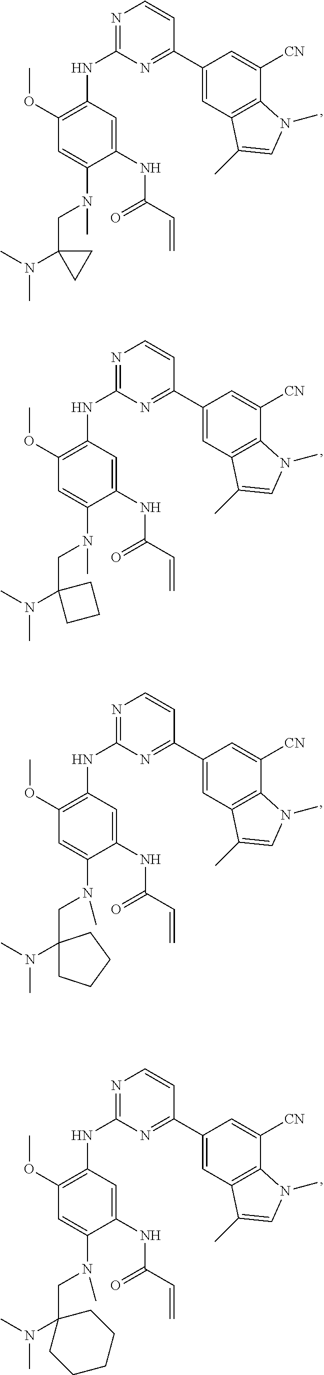

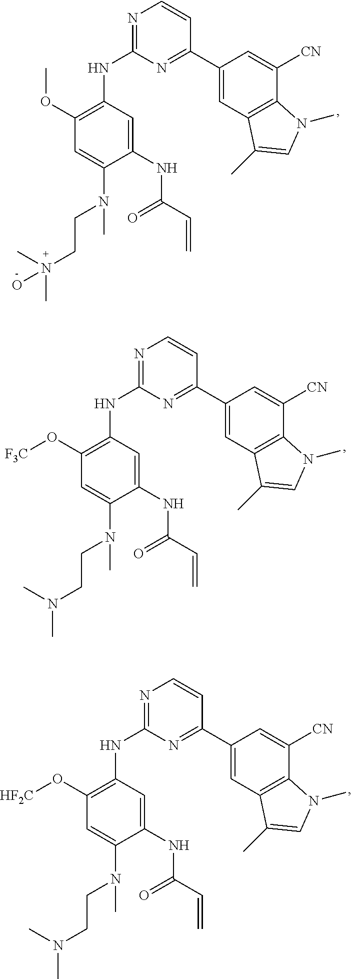

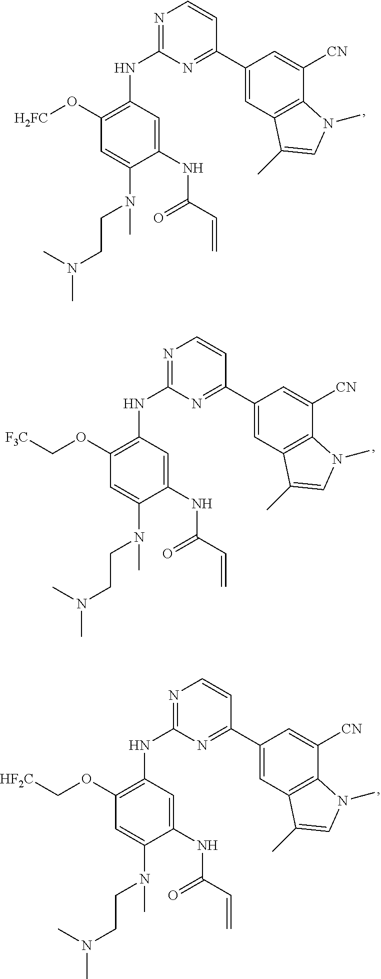

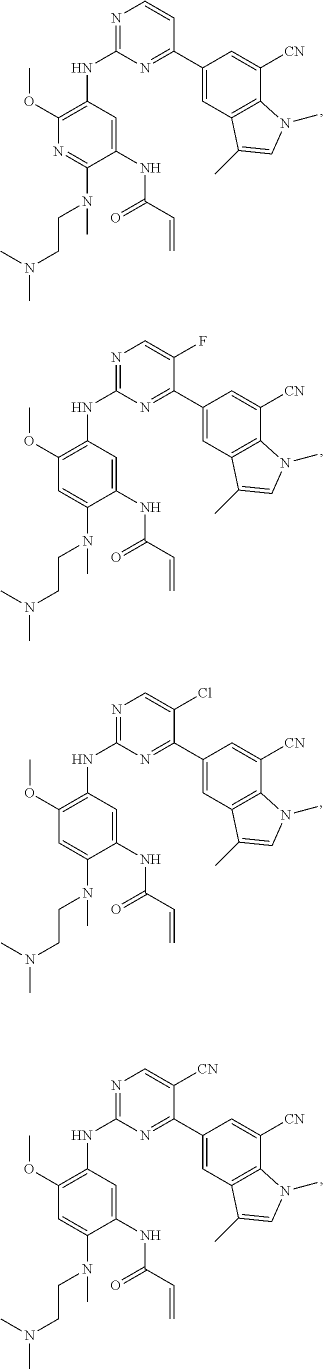

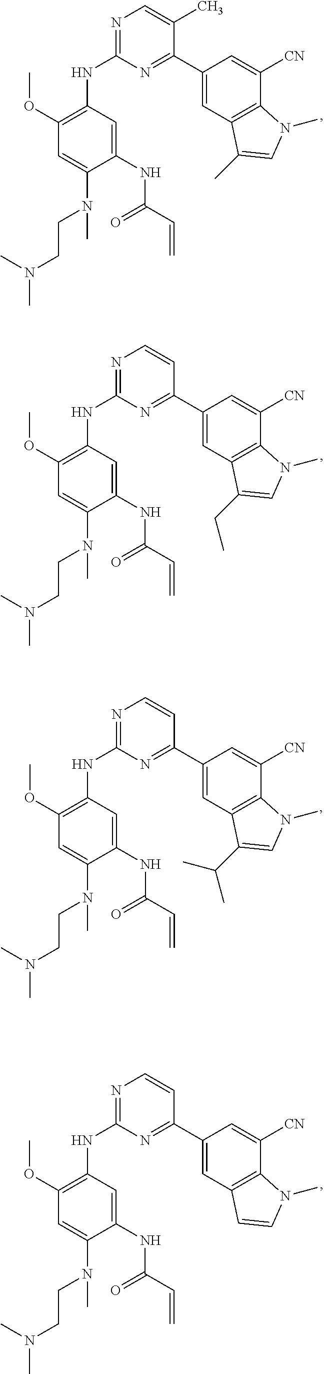

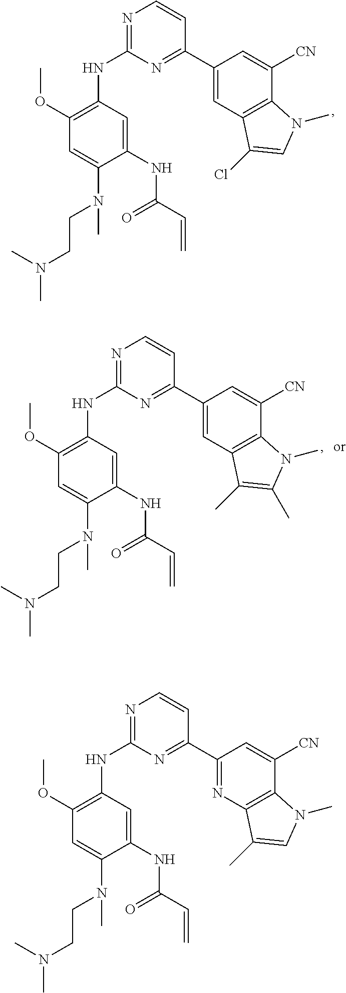

or a stereoisomer or a pharmaceutically acceptable salt, solvate, ester, or prodrug thereof.

In another embodiment, the compound of formula (D), (D-I), (E), (E-I), (F), and/or (G)

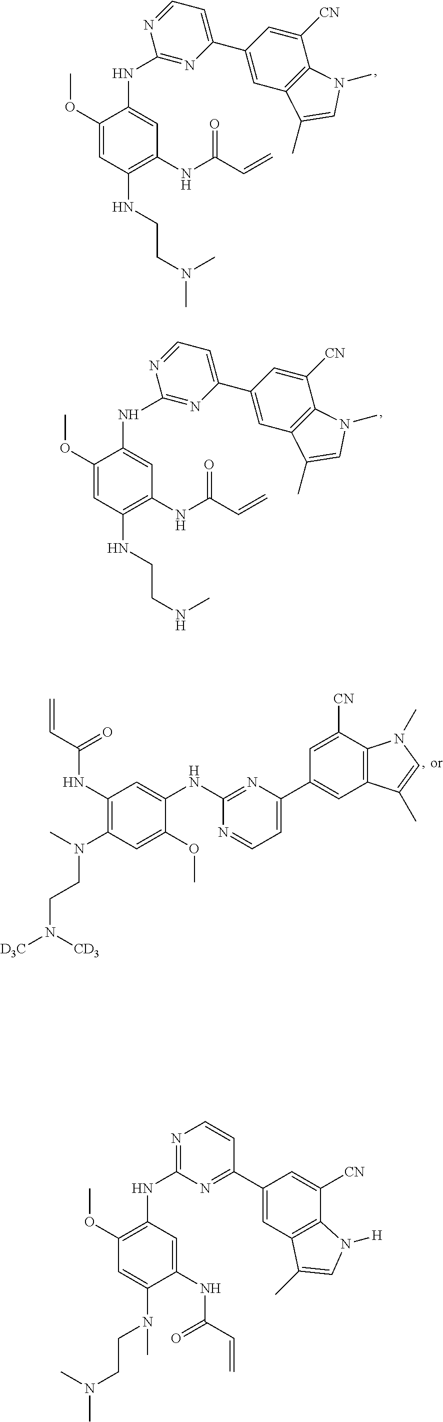

or a stereoisomer or a pharmaceutically acceptable salt, solvate, ester, or prodrug thereof. In another embodiment, the compound is

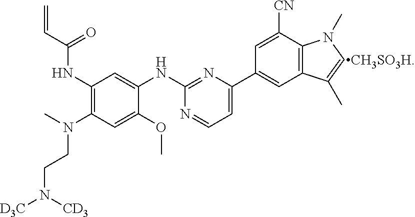 [SMILES: [2H]C([2H])([2H])N(CCN(C)c1cc(OC)c(Nc2nccc(-c3cc(C#N)c4c(c3)c(C)cn4C)n2)cc1NC(=O)C=C)C([2H])([2H])[2H]]

In one embodiment, the present disclosure relates to compounds of formula (E-I):

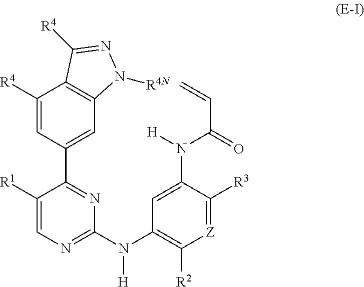

or a stereoisomer or a pharmaceutically acceptable salt, solvate, N-oxide, ester, or prodrug thereof, wherein,
Z is CH or N;
R¹ is hydrogen, fluoro, chloro, bromo, methyl, ethyl, hydroxyl, methoxy, ethoxy, isopropoxy, cyclopropoxy, —OCF₃, —OCH₂CF₃, —OCH₂CHF₂, ethenyl, ethynyl, CF₃, CHF₂, CHO, CH₂OH, CONH₂, CO₂Me, CONHMe, CONMe₂, or cyano;
R² is —OCF₃, —OCHF₂, —OCF₂CF₃, —OCH₂CHF₂, —OCH₂CF₃, cyclopropyl, cyclopropoxy, methoxy, —OCD₃, ethoxy, or isopropoxy;
R³ is N(R¹⁰)C₂₋₆ alkyl-NR¹⁰R¹⁰ or —N(R¹⁰)(C₃₋₁₀ cycloalkylalkyl)-NR¹⁰R¹⁰;
each R⁴ is independently H, cyano, halo, —C₁₋₆ alkyl, —C₁₋₆ haloalkyl, carboxy-C₁₋₆ alkyl, —C₁₋₆ hydroxyalkyl, R⁸R⁹N—C₁₋₆ alkyl-, —C₂₋₆ alkenyl, —C₂₋₆ alkynyl, C₁₋₆ acyl-, R⁷—(CH₂)ₚC(═O)—, C₁₋₆ hydroxyalkyl-C(═O)—, carboxy, —C₁₋₆ alkoxycarbonyl, —C(═O)NR⁸R⁹, hydroxyl, alkoxy, C₁₋₆ acyloxy, —NR⁸R⁹, C₁₋₆ acyl-N(R¹⁰)—, or R⁷SO₂—; and
R⁸ and R⁹ are independently H, —CD₃, C₁₋₆ alkyl, C₃₋₆ alkenyl, C₃₋₆ alkynyl, C₃₋₈ cycloalkyl, C₃₋₈ cycloalkenyl, C₁-C₆ acyl, 4-12 membered monocyclic or bicyclic heterocyclyl, 4-12 membered monocyclic or bicyclic heterocyclyl-C₁-C₆ alkyl-, C₆-C₁₂ aryl, 5-12 membered heteroaryl; and R⁸ and R⁹ may be further independently substituted with up to three substituents chosen from hydroxyl, C₁₋₆ alkoxy, C₁₋₆ hydroxyalkylC₂₋₆ hydroxyalkoxy, oxo, thiono, cyano or halo; or
alternatively, R⁸ and R⁹, taken together with the N atom to which they are both attached, form a heterocyclic ring of 4-7 members, containing up to one other heteroatom selected from O, S, or NR¹¹, or a heterobicyclic ring of 7-12 members which may be fused, bridged or spiro, and contain up to two other heteroatoms chosen from O, S(O)ₓ, or NR¹¹, and these heterocyclic rings are optionally substituted with up to three substituents chosen from hydroxyl, C₁₋₆ alkoxy, C₁₋₆ hydroxyalkyl, C₁₋₆ alkoxy-C₁₋₆ alkyl, C₁₋₆ alkoxy-C₁₋₆ alkoxy, C₂₋₆ hydroxyalkoxy, oxo, thiono, cyano or halo;
R^{4N} is H, —CD₃, or —C₁₋₆ alkyl;
R⁷ is OH, —NR⁸R⁹, —O(CH₂)_{q}NR⁸R⁹, C₁₋₆ alkoxy, or C₂₋₆ hydroxyalkoxy;
each R¹⁰ is independently H, —CD₃, C₁₋₆ alkyl, C₃₋₆ cycloalkyl, C₂₋₆ hydroxyalkyl, C₁₋₆ alkoxy-C₁₋₆ alkyl or C₂₋₆ alkyl-NR⁸R⁹;
alternatively, two R¹⁰ on the same N atom, taken together form a heterocyclic ring of 3-7 members, optionally substituted with up to three substituents chosen from hydroxyl, C₁₋₆ alkoxy, C₁₋₆ hydroxyalkyl, C₁₋₆ alkoxy-C₁₋₆ alkyl, C₁₋₆ alkoxy-C₁₋₆ alkoxy, C₂₋₆ hydroxyalkoxy, oxo, thiono, cyano or halo;
p=0, 1, 2, 3, or 4;
q=2, 3, or 4; and
x=0, 1, or 2.

In some embodiments of the compound of formula (E-I),
R³ is N(R¹⁰)C₂₋₆ alkyl-NR¹⁰R¹⁰ or —N(R¹⁰)(C₃₋₁₀ cycloalkylalkyl)-NR¹⁰R¹⁰;
each R⁴ is independently H, cyano, halo, —C₁₋₆ alkyl, or —C₁₋₆ haloalkyl; and
R^{4N} is H, —CD₃, or —C₁₋₆ alkyl; and
each R¹⁰ is independently H, —CD₃, or —C₁₋₆ alkyl.

In some embodiments of the compound of formula (E-I), the compound is

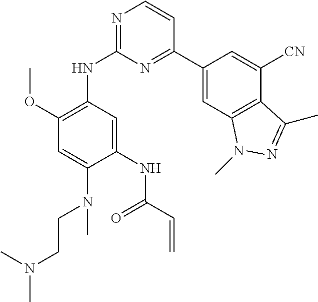

or a stereoisomer or a pharmaceutically acceptable salt, solvate, N-oxide, ester, or prodrug thereof.

In one embodiment, the compound of the present disclosure has the structure of formula (H)

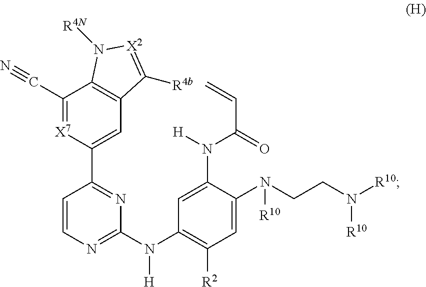

or a stereoisomer or a pharmaceutically acceptable salt, solvate, ester, or prodrug thereof;
wherein,
X⁷ is CH or N;
X² is independently CH, CCH₃, or N;
R² is methoxy, —OCD₃, ethoxy, or isopropoxy;
R^{4b} is H, F, Cl, or CH₃;
R^{4N} is H, —CD₃, CH₃, Et, or CH(CH₃)₂; and
each R¹⁰ is independently H, —CD₃, —CH₃, —CH₂CH₃, or —CH(CH₃)₂.

In one embodiment, the compound of structure (H) comprises
X⁷ is CH or N;
X² is independently CH or CCH₃;
R² is methoxy, —OCD₃, ethoxy, or isopropoxy;
R^{4b} is H, F, Cl, or CH₃;
R^{4N} is H, —CD₃, CH₃, Et, or CH(CH₃)₂; and
each R¹⁰ is independently H, —CD₃, —CH₃, —CH₂CH₃, or —CH(CH₃)₂.

In one embodiment, the compound of the present disclosure has the structure of formula (H-I)

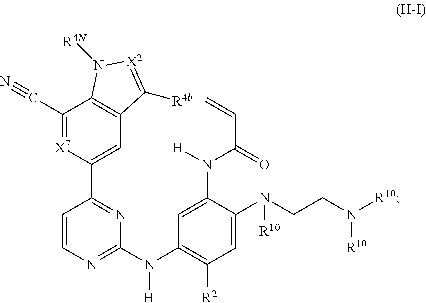

or a stereoisomer or a pharmaceutically acceptable salt, solvate, ester, or prodrug thereof;
wherein,
X⁷ is CH or N;
X² is independently CH, CCH₃, or N;
R² is methoxy, —OCD₃, ethoxy, or isopropoxy;
R^{4b} is H, F, Cl, or CH₃;
R^{4N} is H, —CD₃, CH₃, Et, or CH(CH₃)₂; and
each R¹⁰ is independently —CD₃, —CH₃, —CH₂CH₃, or —CH(CH₃)₂.

In one embodiment, the compound of structure (H) comprises
X⁷ is CH or N;
X² is independently CH or CCH₃;
R² is methoxy, —OCD₃, ethoxy, or isopropoxy;
R^{4b} is H, F, Cl, or CH₃;
R^{4N} is H, —CD₃, CH₃, Et, or CH(CH₃)₂; and
each R¹⁰ is independently H, —CD₃, —CH₃, —CH₂CH₃, or —CH(CH₃)₂.

In one embodiment, R¹⁰ in formula (D), (D-I), (E), (E-I), (F), (G) and/or (H) is H, —CD₃, or —CH₃. In some embodiments, R¹⁰ in formula (D), (D-I), (E), (E-I), (F), (G), (H) and/or (H-I) is —CD₃, or —CH₃. In another embodiment, R¹⁰ in formula (D), (D-I), (E), (E-I), (F), (G), (H) and/or (H-I) is —CH₃.

In one embodiment, R² in formula (D), (D-I), (E), (E-I), (F), (G), (H) and/or (H-I) is methoxy, —OCD₃, ethoxy, or isopropoxy. In another embodiment, R² is methoxy.

In one embodiment, R^{4b} in formula (D), (D-I), (E), (E-I), (F), (G), (H) and/or (H-I) is H or CH₃. In another embodiment, R^{4N} in formula (D), (D-I), (E), (E-I), (F), (G), (H) and/or (H-I) is H or CH₃.

In one embodiment, X⁷ in formula (D), (D-I), (E), (E-I), (F), (G), (H) and/or (H-I) is CH. In another embodiment, X⁷ is N.

In one embodiment, X² in formula (D), (D-I), (E), (E-I), (F), (G), (H) and/or (H-I) is CH. In another embodiment, X² is N.

In one embodiment, X² in formula (H) and/or (H-I) is CH or CCH₃.

In one embodiment, R¹⁰ in formula (H) is H, —CD₃, or —CH₃. In some embodiments, R¹⁰ in formula (H) and/or (H-I) is —CD₃, or —CH₃. In another embodiment, R¹⁰ in formula (H) and/or (H-I) is —CH₃.

In one embodiment, R² in formula (H) and/or (H-I) is methoxy, —OCD₃, ethoxy, or isopropoxy. In another embodiment, R² is methoxy.

In one embodiment, R^{4b} in formula (H) and/or (H-I) is H or CH₃. In another embodiment, R^{4N} in formula (H) and/or (H-I) is H or CH₃.

In one embodiment, X⁷ in formula (H) and/or (H-I) is CH. In another embodiment, X⁷ is N.

In one embodiment, X² in formula (H) and/or (H-I) is CH. In another embodiment, X² is N.

In one embodiment of formula (H),
X⁷ is CH or N;
X² is independently CH or CCH₃;
R² is methoxy, —OCD₃, ethoxy, or isopropoxy;
R^{4b} is H, F, Cl, or CH₃;
R^{4N} is H, —CD₃, CH₃, Et, or CH(CH₃)₂; and
each R¹⁰ is independently H, —CD₃, —CH₃, —CH₂CH₃, or —CH(CH₃)₂.

In one embodiment of the compound of formula (H) and/or (H-I),
X⁷ is CH;
X² is CH;
R² is methoxy, —OCD₃, ethoxy, or isopropoxy;
R^{4b} is H, F, Cl, or CH₃;
R^{4N} is H, —CD₃, CH₃, Et, or CH(CH₃)₂; and
each R¹⁰ is independently —CD₃, —CH₃, —CH₂CH₃, or —CH(CH₃)₂.

In one embodiment of the compound of formula (H) and/or (H-I),
X⁷ is CH;
X² is CH;
R² is methoxy, —OCD₃, ethoxy, or isopropoxy;
R^{4b} is F, Cl, or CH₃;
R^{4N} is —CD₃, CH₃, Et, or CH(CH₃)₂; and
each R¹⁰ is independently —CD₃, —CH₃, —CH₂CH₃, or —CH(CH₃)₂.

In one embodiment, the compound of the present disclosure has the structure of formula (J):

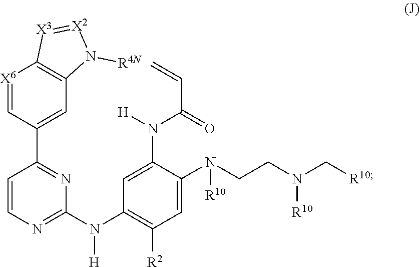

or a stereoisomer or a pharmaceutically acceptable salt, solvate, ester, or prodrug thereof;
wherein,
X⁶ is N or C—R⁴, wherein R⁴ is H, cyano, CONH₂, CONHCH₃, CON(CH₃)₂, COCH₃;
X² is independently C—H, C—CH₃ or N;
X³ is independently C—H, C—CH₃, C—CF₃, C—CHF₂, C—F, C—Cl, or N;
R^{4N} is H, —CD₃, —CH₃, —CH₂CH₃, or —CH(CH₃)₂;
R² is —OCF₃, —OCHF₂, —OCF₂CF₃, —OCH₂CHF₂, —OCH₂CF₃, cyclopropyl, cyclopropoxy, methoxy, —OCD₃, ethoxy, or isopropoxy;
each R¹⁰ is independently H, —CD₃, C₁₋₆ alkyl, C₃₋₆ cycloalkyl, C₂₋₆ hydroxyalkyl, C₁₋₆ alkoxy-C₁₋₆ alkyl or C₂₋₆ alkyl-NR⁸R⁹; and
R⁸ and R⁹ are independently H, —CD₃, C₁₋₆ alkyl, C₃₋₆ alkenyl, C₃₋₆ alkynyl, C₃₋₈ cycloalkyl, C₃₋₈ cycloalkenyl, C₁-C₆ acyl, 4-12 membered monocyclic or bicyclic heterocyclyl, 4-12 membered monocyclic or bicyclic heterocyclyl-C₁-C₆ alkyl-, C₆-C₁₂ aryl, 5-12 membered heteroaryl; and R⁸ and R⁹ may be further independently substituted with up to three substituents chosen from hydroxyl, C₁₋₆ alkoxy, C₁₋₆ hydroxyalkylC₂₋₆ hydroxyalkoxy, oxo, thiono, cyano or halo.

In one embodiment, R¹⁰ in formula (J) is each —CD₃, C₁₋₆ alkyl, C₃₋₆ cycloalkyl, C₂₋₆ hydroxyalkyl, C₁₋₆ alkoxy-C₁₋₆ alkyl or C₂₋₆ alkyl-NR⁸R⁹.

In one embodiment, a compound of formula (J) comprises:
X⁶ is C—CN;
X² is C—H or C—CH₃;
X³ is C—H or C—CH₃;
R^{4N} is H, —CD₃, —CH₃, —CH₂CH₃, or —CH(CH₃)₂;
R² is methoxy, —OCD₃, ethoxy, or isopropoxy; and
each R¹⁰ is independently H, —CD₃, —CH₃, —CH₂CH₃, or —CH(CH₃)₂.

In one embodiment, X⁶ in formula (J) is C—CN. In another embodiment, X² in formula (J) is C—H or C—CH₃. In another embodiment, X³ in formula (J) is C—H or C—CH₃.

In some embodiments, R^{4N} in formula (J) is H, —CD₃, —CH₃, —CH₂CH₃, or —CH(CH₃)₂. In other embodiments, R^{4N} is H, or —CH₃.

In one embodiment, R² in formula (J) is methoxy, —OCD₃, ethoxy, or isopropoxy. In another embodiment, R² is methoxy.

In some embodiments, R¹⁰ in formula (J) is each independently H, —CD₃, —CH₃, —CH₂CH₃, or —CH(CH₃)₂. In another embodiment, R¹⁰ is —CD₃, —CH₃, —CH₂CH₃, or —CH(CH₃)₂. In other embodiments, R¹⁰ is —CH₃.

In one embodiment, the compound of the present disclosure has the structure of formula (K):

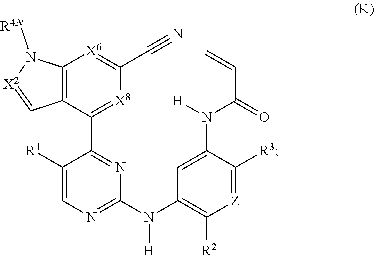

or a stereoisomer or a pharmaceutically acceptable salt, solvate, ester, or prodrug thereof;
wherein,
Z is CH or N;
X² is CR^{4a} or N;
X⁶ is CR⁴b or N;
X⁸ is CH or N;
R¹ is hydrogen, methyl, fluoro, chloro, bromo, CF₃, or cyano;
R² is —OCF₃, —OCHF₂, —OCF₂CF₃, —OCH₂CHF₂, —OCH₂CF₃, cyclopropoxy, methoxy, —OCD₃, ethoxy, or isopropoxy;
R³ is N(R¹⁰)C₂₋₆ alkyl-NR¹⁰R¹⁰;
R^{4a} is H, cyano, halo, —C₁₋₆ alkyl, or —C₁₋₆ haloalkyl;
R^{4b} is H, cyano, nitro, halo, —C₁₋₆ alkyl, —C₁₋₆ haloalkyl, carboxy-C₁₋₆ alkyl, —C₁₋₆ hydroxyalkyl, R⁸R⁹N—C₁₋₆ alkyl-, —C₂₋₆ alkenyl, —C₂₋₆ alkynyl, C₁₋₆ acyl-, R⁷—(CH₂)ₚC(═O)—, C₁₋₆ hydroxyalkyl-C(═O)—, carboxy, —C₁₋₆ alkoxycarbonyl, —C(═O)NR⁸R⁹, hydroxyl, alkoxy, —OCD₃, C₁₋₆ acyloxy, —NR⁸R⁹, C₁₋₆ acyl-N(R¹⁰)—, or R⁷SO₂—;
R^{4N} is H, —C₁₋₆ alkyl, or —CD₃;
R⁸ and R⁹ are independently H, —CD₃, C₁₋₆ alkyl, C₃₋₈ cycloalkyl, C₃₋₈ cycloalkyl-(C₁₋₃ alkyl)-, C₁-C₆ acyl, phenyl, monocyclic heteroaryl, or monocyclic heterocyclyl; and R⁸ and R⁹ may be further independently substituted with up to three substituents chosen from hydroxyl, C₁₋₆ alkoxy, oxo, thiono, cyano or halo; or
alternatively, R⁸ and R⁹, taken together with the N atom to which they are both attached, form a heterocyclic ring of 4-7 members, containing up to one other heteroatom selected from O, S, or NR¹¹, or a heterobicyclic ring of 7-12 members which may be fused, bridged or spiro, and contain up to two other heteroatoms chosen from O, S(O)ₓ, or NR¹¹, and these heterocyclic rings are optionally substituted with up to three substituents chosen from hydroxyl, C₁₋₆ alkoxy, C₁₋₆ hydroxyalkyl, C₁₋₆ alkoxy-C₁₋₆ alkyl, C₁₋₆ alkoxy-C₁₋₆ alkoxy, C₂₋₆ hydroxyalkoxy, oxo, thiono, cyano or halo;
each R¹⁰ is independently H, —CD₃, C₁₋₆ alkyl, C₃₋₆ cycloalkyl, C₂₋₆ hydroxyalkyl, C₁₋₆ alkoxy-C₁₋₆ alkyl or C₂₋₆ alkyl-NR⁸R⁹;
p=0, 1, 2, 3, or 4;
q=2, 3, or 4; and
x=0, 1, or 2.

In one embodiment, R³ in formula (K) is N(R¹⁰)C₂₋₆ alkyl-NR¹⁰R¹⁰. In one embodiment, R³ in formula (K) is N(R¹⁰)C₂₋₆ alkyl-NR¹⁰R¹⁰, wherein R¹⁰ is —CD₃, C₁₋₆ alkyl, C₃₋₆ cycloalkyl, C₂₋₆ hydroxyalkyl, C₁₋₆ alkoxy-C₁₋₆ alkyl or C₂₋₆ alkyl-NR⁸R⁹. In one embodiment, R³ is —N(CH₃)CH₂CH₂NR¹⁰R¹⁰. In one embodiment, R³ is —N(CH₃)CH₂CH₂NR¹⁰R¹⁰, wherein R¹⁰ is —CD₃, C₁₋₆ alkyl, C₃₋₆ cycloalkyl, C₂₋₆ hydroxyalkyl, C₁₋₆ alkoxy-C₁₋₆ alkyl or C₂₋₆ alkyl-NR⁸R⁹.

In one embodiment, the compound of the present disclosure has the structure of formula (L):

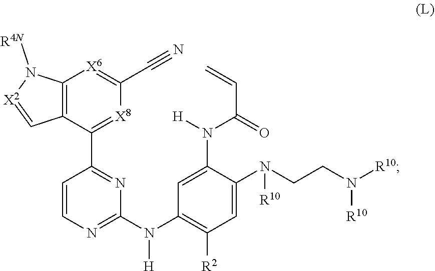

or a stereoisomer or a pharmaceutically acceptable salt, solvate, ester, or prodrug thereof;
wherein,
X² is CR^{4a} or N;
X⁶ is CR⁴b or N;
X⁸ is CH or N;
R² is methoxy, —OCD₃, ethoxy, or isopropoxy;
R^{4a} is H, cyano, halo, —C₁₋₆ alkyl, or —C₁₋₆ haloalkyl;
R^{4b} is H, cyano, nitro, halo, —C₁₋₆ alkyl, —C₁₋₆ haloalkyl, carboxy-C₁₋₆ alkyl, —C₁₋₆ hydroxyalkyl, R⁸R⁹N—C₁₋₆ alkyl-, —C₂₋₆ alkenyl, —C₂₋₆ alkynyl, C₁₋₆ acyl-, R⁷—(CH₂)ₚC(═O)—, C₁₋₆ hydroxyalkyl-C(═O)—, carboxy, —C₁₋₆ alkoxycarbonyl, —C(═O)NR⁸R⁹, hydroxyl, alkoxy, —OCD₃, C₁₋₆ acyloxy, —NR⁸R⁹, C₁₋₆ acyl-N(R¹⁰)—, R⁷SO₂—;
R^{4N} is H, —CH₃, Et, CH(CH₃)₂, or —CD₃;
R⁸ and R⁹ are independently H, —CD₃, C₁₋₆ alkyl, C₃₋₈ cycloalkyl, C₃₋₈ cycloalkyl-(C₁₋₃ alkyl)-, C₁-C₆ acyl, phenyl, monocyclic heteroaryl, or monocyclic heterocyclyl; and R⁸ and R⁹ may be further independently substituted with up to three substituents chosen from hydroxyl, C₁₋₆ alkoxy, oxo, thiono, cyano or halo; or
alternatively, R⁸ and R⁹, taken together with the N atom to which they are both attached, form a heterocyclic ring of 4-7 members, containing up to one other heteroatom selected from O, S, or NR¹¹, or a heterobicyclic ring of 7-12 members which may be fused, bridged or spiro, and contain up to two other heteroatoms chosen from O, S(O)ₓ, or NR¹¹, and these heterocyclic rings are optionally substituted with up to three substituents chosen from hydroxyl, C₁₋₆ alkoxy, C₁₋₆ hydroxyalkyl, C₁₋₆ alkoxy-C₁₋₆ alkyl, C₁₋₆ alkoxy-C₁₋₆ alkoxy, C₂₋₆ hydroxyalkoxy, oxo, thiono, cyano or halo;
each R¹⁰ is independently H, —CD₃, C₁₋₆ alkyl, C₃₋₆ cycloalkyl, C₂₋₆ hydroxyalkyl, C₁₋₆ alkoxy-C₁₋₆ alkyl or C₂₋₆ alkyl-NR⁸R⁹;
p=0, 1, 2, 3, or 4;
q=2, 3, or 4; and
x=0, 1, or 2.

In one embodiment, the compound of formula (L) comprises:
X² is CR^{4a} or N;
X⁶ is CR⁴b or N;
X⁸ is CH or N;
R² is methoxy, —OCD₃, ethoxy, or isopropoxy;
R^{4a} is H, F, Cl, CH₃, CF₃, or CHF₂;
R^{4b} is H, cyano, nitro, halo, —C₁₋₆ alkyl, or —C₁₋₆ haloalkyl;
R^{4N} is H, —CD₃, —CH₃, —CH₂CH₃, or —CH(CH₃)₂; and
each R¹⁰ is independently H, —CD₃, —CH₃, —CH₂CH₃, or —CH(CH₃)₂.

In another embodiment, the compound of formula (L) comprises:
X² is CR^{4a} or N;
X⁶ is CR⁴b;
X⁸ is CH;
R² is methoxy, —OCD₃, ethoxy, or isopropoxy;
R^{4a} is H, F, CH₃, CF₃, or CHF₂;
R^{4b} is independently H, CH₃, F, Cl, CF₃, or CHF₂;
R^{4N} is H, —CD₃, —CH₃, —CH₂CH₃, or —CH(CH₃)₂;
each R¹⁰ is independently H, —CD₃, —CH₃, —CH₂CH₃, or —CH(CH₃)₂.

In one embodiment, each R¹⁰ in formula (L) is independently —CD₃, C₁₋₆ alkyl, C₃₋₆ cycloalkyl, C₂₋₆ hydroxyalkyl, C₁₋₆ alkoxy-C₁₋₆ alkyl or C₂₋₆ alkyl-NR⁸R⁹. In another embodiment, R¹⁰ is-CD₃, —CH₃, —CH₂CH₃, or —CH(CH₃)₂.

In one embodiment, X² in formula (K) and/or (L) is CH or N.

In one embodiment, X⁶ in formula (K) and/or (L) is CH or N. In some embodiments, X⁶ is CH.

In one embodiment, X⁸ in formula (K) and/or (L) is CH or N. In some embodiments, X⁸ is CH.

In one embodiment, R^{4N} in formula (K) and/or (L) is H, —CD₃, or —CH₃.

In one embodiment, R² in formula (K) and/or (L) is methoxy, —OCD₃, ethoxy, or isopropoxy. In another embodiment, R² is methoxy.

In some embodiments, R¹⁰ in formula (K) and/or (L) is each independently H, —CD₃, —CH₃, —CH₂CH₃, or —CH(CH₃)₂. In other embodiments, R¹⁰ is each independently —CD₃, —CH₃, —CH₂CH₃, or —CH(CH₃)₂. In other embodiments, R¹⁰ is —CH₃.

In one embodiment, the compound of the present disclosure has the structure of formula (M):

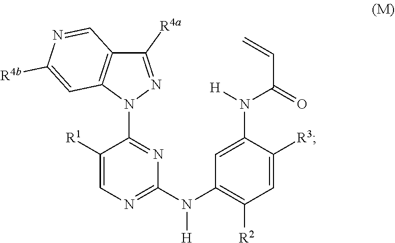

or a stereoisomer or a pharmaceutically acceptable salt, solvate, ester, or prodrug thereof; wherein,
Z is CH or N;
R¹ is hydrogen, methyl, fluoro, chloro, bromo, —CF₃, or cyano;
R² is —OCF₃, —OCHF₂, —OCF₂CF₃, —OCH₂CHF₂, —OCH₂CF₃, cyclopropoxy, methoxy, —OCD₃, ethoxy, or isopropoxy;
R³ is N(R¹⁰)C₂₋₆ alkyl-NR¹⁰R¹⁰;
R^{4a} is cyano, —C₁₋₆ hydroxyalkyl, C₁₋₆ acyl-, pyrazole, 1,2,3-triazole, tetrazole, —C(═O)NR⁸R⁹, —NR⁸R⁹, C₁₋₆ acyl-N(R¹⁰)—, (C₁₋₃ alkyl)SO₂NH—, (C₁₋₆ alkyl)SO₂—, or R⁷SO₂—;
R^{4b} is H, cyano, halo, —C₁₋₆ alkyl, or —C₁₋₆ haloalkyl;
R⁷ is —OH or —NR⁸R⁹;
R⁸ and R⁹ are independently H, —CD₃, C₁₋₆ alkyl, C₃₋₈ cycloalkyl, C₃₋₈ cycloalkyl-(C₁₋₃ alkyl)-, C₁-C₆ acyl, phenyl, monocyclic heteroaryl, or monocyclic heterocyclyl; and R⁸ and R⁹ may be further independently substituted with up to three substituents chosen from hydroxyl, C₁₋₆ alkoxy, oxo, thiono, cyano or halo; or
alternatively, R⁸ and R⁹, taken together with the N atom to which they are both attached, form a heterocyclic ring of 4-7 members, containing up to one other heteroatom chosen from O, S, or NR¹¹,
each R¹⁰ is independently H, —CD₃, C₁₋₆ alkyl, C₃₋₆ cycloalkyl, C₂₋₆ hydroxyalkyl, C₂₋₆ alkyl-NR⁸R⁹;
alternatively, two R¹⁰ on the same N atom to which they are both attached, form a heterocyclic ring of 5-6 members, containing up to one other heteroatom selected from O, S, or NR¹¹; and
each R¹¹ is independently hydrogen or C₁-C₆ alkyl, which is optionally substituted with up to three substituents selected from hydroxyl, oxo, thiono, cyano and halo.

In one embodiment, a compound of formula (M) comprises:
Z is CH;
R¹ is hydrogen, methyl, fluoro, chloro, bromo, —CF₃, or cyano;
R² is methoxy, —OCD₃, ethoxy, or isopropoxy;
R³ is —N(CH₃)CH₂CH₂NR¹⁰R¹⁰;
R^{4a} is —NR⁸R⁹;
R^{4b} is H, CH₃, F, Cl, CF₃, or CHF₂;
R⁸ and R⁹ are independently H, —CD₃, C₁₋₆ alkyl, C₃₋₈ cycloalkyl, C₃₋₈ cycloalkyl-(C₁₋₃ alkyl)-, C₁-C₆ acyl, phenyl, monocyclic heteroaryl, or monocyclic heterocyclyl; and R⁸ and R⁹ may be further independently substituted with up to three substituents chosen from hydroxyl, C₁₋₆ alkoxy, oxo, thiono, cyano or halo; and
each R¹⁰ is independently H, —CD₃, —CH₃, —CH₂CH₃, or —CH(CH₃)₂.

In one embodiment, R¹⁰ in formula (M) is each independently —CD₃, C₁₋₆ alkyl, C₃₋₆ cycloalkyl, C₂₋₆ hydroxyalkyl, C₂₋₆ alkyl-NR⁸R⁹. In another embodiment, R¹⁰ in formula (M) is each independently H, —CD₃, —CH₃, —CH₂CH₃, or —CH(CH₃)₂. In other embodiments, R¹⁰ is each independently —CD₃, —CH₃, —CH₂CH₃, or —CH(CH₃)₂. In other embodiments, R¹⁰ is each independently H, —CD₃, methyl, ethyl, or isopropyl. In some embodiments, R¹⁰ is each independently —CD₃, methyl, ethyl, or isopropyl. In some embodiments, R¹⁰ is each independently H, —CD₃, or methyl. In other embodiments, R¹⁰ is each independently —CD₃, or methyl.

In another embodiment, R^{4a} in formula (M) is each independently H, —C₁₋₆ alkyl, or —NR⁸R⁹. In one embodiment, R^{4a} is —NR⁸R⁹. In one embodiment, R⁸ and R⁹ are independently H, —CD₃, or C₁₋₆ alkyl. In another embodiment, R^{4a} is —N(CH₃)₂.

In some embodiments, R^{4b} in formula (M) are each independently H, cyano, F, Cl, Br, CH₃, CF₃, or CHF₂. In one embodiment, R^{4b} is H, CH₃, or CF₃.

In one embodiment, R² in formula (M) is methoxy, —OCD₃, ethoxy, or isopropoxy. In another embodiment, R² is methoxy.

In one embodiment, R¹ in formula (M) is H.

In one embodiment, the compound of the present disclosure has the structure of formula (N):

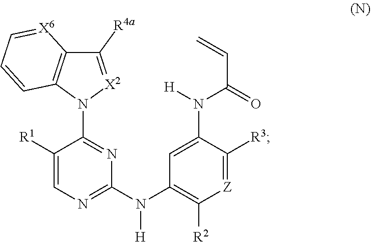

or a stereoisomer or a pharmaceutically acceptable salt, solvate, ester, or prodrug thereof;
wherein,
X² is CH, CCH₃, or N;
X⁶ is CR⁴ or N;
Z is CH or N;
R¹ is hydrogen, methyl, fluoro, chloro, bromo, —CF₃, or cyano;
R² is —OCF₃, —OCHF₂, —OCF₂CF₃, —OCH₂CHF₂, or —OCH₂CF₃;
R³ is N(R¹⁰)C₂₋₆ alkyl-NR¹⁰R¹⁰;
R⁴ is H, cyano, halo, —C₁₋₆ alkyl, —C₁₋₆ haloalkyl;
R^{4a} is independently cyano, —C₁₋₆ hydroxyalkyl, C₁₋₆ acyl-, pyrazole, 1,2,3-triazole, tetrazole, —C(═O)NR⁸R⁹, —NR⁸R⁹, C₁₋₆ acyl-N(R¹⁰)—, (C₁₋₃ alkyl)SO₂NH—, (C₁₋₆ alkyl)SO₂—, or R⁷SO₂—;
R⁷ is —OH or —NR⁸R⁹;
R⁸ and R⁹ are independently H, —CD₃, C₁₋₆ alkyl, C₃₋₈ cycloalkyl, C₃₋₈ cycloalkyl-(C₁₋₃ alkyl)-, C₁-C₆ acyl, phenyl, monocyclic heteroaryl, or monocyclic heterocyclyl; and R⁸ and R⁹ may be further independently substituted with up to three substituents chosen from hydroxyl, C₁₋₆ alkoxy, oxo, thiono, cyano or halo;
each R¹⁰ is independently H, —CD₃, C₁₋₆ alkyl, C₃₋₆ cycloalkyl, C₂₋₆ hydroxyalkyl, or C2-6 alkyl-NR⁸R⁹.

In one embodiment, R³ in formula (N) is —N(CH₃)CH₂CH₂NRR¹. In one embodiment, R³ in formula (N) is —N(CH₃)CH₂CH₂NRR¹, wherein R¹⁰ is independently —CD₃, C₁₋₆ alkyl, C₃₋₆ cycloalkyl, C₂₋₆ hydroxyalkyl, or C₂₋₆ alkyl-NR⁸R⁹.

In one embodiment, R^{4a} in formula (N) is —NR⁸R⁹.

In one embodiment, R¹ in formula (N) is H.

In one embodiment, the compound of the present disclosure has the structure of formula (O):

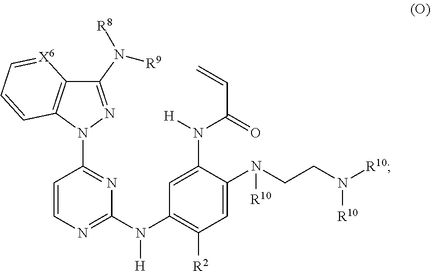

or a stereoisomer or a pharmaceutically acceptable salt, solvate, ester, or prodrug thereof; wherein,
X⁶ is CH, CCH₃, or N;
R² is —OCF₃, —OCHF₂, —OCF₂CF₃, —OCH₂CHF₂, or —OCH₂CF₃;
R⁸ and R⁹ are each independently H, —CD₃, —CH₃, —CH₂CH₃, or —CH(CH₃)₂; and
each R¹⁰ is independently H, —CD₃, —CH₃, —CH₂CH₃, or —CH(CH₃)₂.

In another embodiment, R¹⁰ in formula (N) and/or (O) is each independently H, —CD₃, —CH₃, —CH₂CH₃, or —CH(CH₃)₂. In other embodiments, R¹⁰ is each independently H, —CD₃, methyl, ethyl, or isopropyl. In some embodiments, R¹⁰ is each independently H, —CD₃, or methyl.

In another embodiment, R¹⁰ in formula (N) and/or (O) is each independently —CD₃, —CH₃, —CH₂CH₃, or —CH(CH₃)₂. In other embodiments, R¹⁰ is each independently —CD₃, methyl, ethyl, or isopropyl. In some embodiments, R¹⁰ is each independently —CD₃, or methyl.

In one embodiment, R⁸ and R⁹ in formula (N) and/or (O) are each independently H, —CD₃, or C₁₋₆ alkyl. In another embodiment, R⁸ and R⁹ is each H, methyl, or ethyl.

In one embodiment, R² in formula (N) and/or (O) is —OCF₃, —OCHF₂, —OCF₂CF₃, —OCH₂CHF₂, or —OCH₂CF₃. In another embodiment, R² is —OCF₃ or —OCH₂CHF₂.

In one embodiment, the compound of the present disclosure has the structure of formula (P):

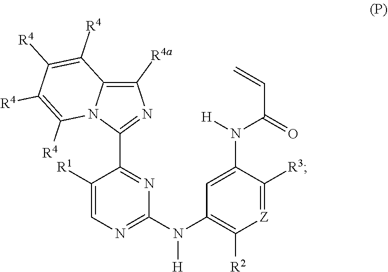

or a stereoisomer or a pharmaceutically acceptable salt, solvate, ester, tautomer, or prodrug thereof;
wherein:
Z is CH or N;
R¹ is independently selected from hydrogen, fluoro, chloro, bromo, methyl, ethyl, hydroxyl, methoxy, ethoxy, isopropoxy, cyclopropoxy, —OCF₃, —OCH₂CF₃, —OCH₂CHF₂, ethenyl, ethynyl, CF₃, CHF₂, CHO, CH₂OH, CONH₂, CO₂Me, CONHMe, CONMe₂, or cyano;
R² is —OCF₃, —OCHF₂, —OCF₂CF₃, —OCH₂CHF₂, —OCH₂CF₃, cyclopropyl, cyclopropoxy, methoxy, —OCD₃, ethoxy, or isopropoxy;
R³ is N(R¹⁰)C₂₋₆ alkyl-NR¹⁰R¹⁰, N(R¹⁰)C₂₋₆ alkyl-R⁷, O(CH₂)ₚR⁷, N(R¹⁰)C(═O)(CH₂)ₚR⁷ or R⁷;
each R⁴ is independently H, cyano, nitro, halo, —C₁₋₆ alkyl, —C₁₋₆ haloalkyl, carboxy-C₁₋₆ alkyl, —C₁₋₆ hydroxyalkyl, R⁸R⁹N—C₁₋₆ alkyl-, —C₂₋₆ alkenyl, —C₂₋₆ alkynyl, C₁₋₆ acyl-, R⁷⁻(CH₂)ₚC(═O)—, C₁₋₆ hydroxyalkyl-C(═O)—, carboxy, —C₁₋₆ alkoxycarbonyl, —C(═O)NR⁸R⁹, hydroxyl, alkoxy, C₁₋₆ acyloxy, —NR⁸R⁹, C₁₋₆ acyl-N(R¹⁰)—, or R⁷SO₂—;
R^{4a} is independently H, cyano, nitro, halo, —C₁₋₆ alkyl, —C₁₋₆ haloalkyl, —C₁₋₆ alkoxy, —C₁₋₆ haloalkoxy, —C₁₋₆ hydroxyalkyl, C₁₋₆ acyl-, pyrazole, 1,2,3-triazole, tetrazole, —C(═O)NR⁸R⁹, —NR⁸R⁹, C₁₋₆ acyl-N(R¹⁰)—, (C₁₋₃ alkyl)SO₂NH—, (C₁₋₆ alkyl)SO₂—, or R⁷SO₂—;
R⁷ is OH, NR⁸R⁹, O(CH₂)_{q}NR⁸R⁹, C₁₋₆ alkoxy, or C₂₋₆ hydroxyalkoxy;
R⁸ and R⁹ are independently H, —CD₃, C₁₋₆ alkyl, C₃₋₆ alkenyl, C₃₋₆ alkynyl, C₃₋₈ cycloalkyl, C₃₋₈ cycloalkenyl, C₁-C₆ acyl, 4-12 membered monocyclic or bicyclic heterocyclyl, 4-12 membered monocyclic or bicyclic heterocyclyl-C₁-C₆ alkyl-, C₆-C₁₂ aryl, 5-12 membered heteroaryl; and R⁸ and R⁹ may be further independently substituted with up to three substituents chosen from hydroxyl, C₁₋₆ alkoxy, C₁₋₆ hydroxyalkylC₂₋₆ hydroxyalkoxy, oxo, thiono, cyano or halo; or
alternatively, R⁸ and R⁹, taken together with the N atom to which they are both attached, form a heterocyclic ring of 4-7 members, containing up to one other heteroatom chosen from O, S, or NR¹¹, or a heterobicyclic ring of 7-12 members which may be fused, bridged or spiro, and contain up to two other heteroatoms chosen from O, S(O)ₓ, or NR¹¹, and these heterocyclic rings are optionally substituted with up to three substituents chosen from hydroxyl, C₁₋₆ alkoxy, C₁₋₆ hydroxyalkyl, C₁₋₆ alkoxy-C₁₋₆ alkyl, C₁₋₆ alkoxy-C₁₋₆ alkoxy, C₂₋₆ hydroxyalkoxy, oxo, thiono, cyano or halo;
each R¹⁰ is independently H, —CD₃, C₁₋₆ alkyl, C₃₋₆ cycloalkyl, C₂₋₆ hydroxyalkyl, C₁₋₆ alkoxy-C₁₋₆ alkyl or C₂₋₆ alkyl-NR⁸R⁹; or
alternatively, two R¹⁰ on the same N atom to which they are both attached, form a heterocyclic ring of 5-6 members, containing up to one other heteroatom selected from O, S, or NR¹¹; and
each R¹¹ is independently hydrogen or C₁-C₆ alkyl, which is optionally substituted with up to three substituents selected from hydroxyl, oxo, thiono, cyano and halo;
p=0, 1, 2, 3, or 4;
q=2, 3, or 4; and
x=0, 1, or 2.

In one embodiment, the compound of formula (P) comprises:
Z is CH or N;
R¹ is hydrogen, methyl, fluoro, chloro, bromo, —CF₃, or cyano;
R³ is N(R¹⁰)C₂₋₆ alkyl-NR¹⁰R¹⁰;
each R⁴ is independently H, cyano, halo, —C₁₋₆ alkyl, —C₁₋₆ haloalkyl;
R^{4a} is independently H, cyano, nitro, halo, —C₁₋₆ alkyl, —C₁₋₆ haloalkyl, —C₁₋₆ alkoxy, —C₁₋₆ haloalkoxy, —C(═O)NR⁸R⁹, or —NR⁸R⁹;
R⁸ and R⁹ are independently H, —CD₃, —CH₃, —CH₂CH₃, or —CH(CH₃)₂; and
each R¹⁰ is independently H, —CD₃, —CH₃, —CH₂CH₃, or —CH(CH₃)₂.

In one embodiment, R¹ in formula (P) is hydrogen, methyl, fluoro, chloro, bromo, —CF₃, or cyano. In one embodiment, R¹ is H.

In one embodiment, R³ in formula (P) is N(R¹⁰)C₂₋₆ alkyl-NR¹⁰R¹⁰. In one embodiment, R³ in formula (P) is N(R¹⁰)C₂₋₆ alkyl-NR¹⁰R¹⁰, wherein each R¹⁰ is independently —CD₃, C₁₋₆ alkyl, C₃₋₆ cycloalkyl, C₂₋₆ hydroxyalkyl, C₁₋₆ alkoxy-C₁₋₆ alkyl or C₂₋₆ alkyl-NR⁸R⁹; In another embodiment, R³ is —N(CH₃)CH₂CH₂NR¹⁰R¹⁰. In another embodiment, R³ is —N(CH₃)CH₂CH₂NR¹⁰R¹⁰, wherein each R¹⁰ is independently —CD₃, C₁₋₆ alkyl, C₃₋₆ cycloalkyl, C₂₋₆ hydroxyalkyl, C₁₋₆ alkoxy-C₁₋₆ alkyl or C₂₋₆ alkyl-NR⁸R⁹;

In one embodiment, each R⁴ in formula (P) is independently H, cyano, halo, —C₁₋₆ alkyl, —C₁₋₆ haloalkyl. In one embodiment, each R⁴ is independently H, cyano, halo, or methyl.

In one embodiment, R^{4a} in formula (P) is H, cyano, nitro, halo, —C₁₋₆ alkyl, —C₁₋₆ haloalkyl, —C₁₋₆ alkoxy, —C₁₋₆ haloalkoxy, —C(═O)NR⁸R⁹, or —NR⁸R⁹. In another embodiment, R^{4a} is H, —C₁₋₆ alkyl, or —NR⁸R⁹. In one embodiment, R^{4a} is —NR⁸R⁹. In one embodiment, R⁸ and R⁹ are independently H, —CD₃, —CH₃, —CH₂CH₃, or —CH(CH₃)₂. In another embodiment, R^{4a} is —N(CH₃)₂.

In another embodiment, R¹⁰ in formula (P) is each independently H, —CD₃, —CH₃, —CH₂CH₃, or —CH(CH₃)₂. In other embodiments, R¹⁰ is each independently H, —CD₃, methyl, ethyl, or isopropyl. In some embodiments, R¹⁰ is each independently H, —CD₃, or methyl.

In another embodiment, R¹⁰ in formula (P) is each independently —CD₃, —CH₃, —CH₂CH₃, or —CH(CH₃)₂. In other embodiments, R¹⁰ is each independently —CD₃, methyl, ethyl, or isopropyl. In some embodiments, R¹⁰ is each independently —CD₃, or methyl.

In one embodiment, the present disclosure relates to one or more of the following compounds selected from:

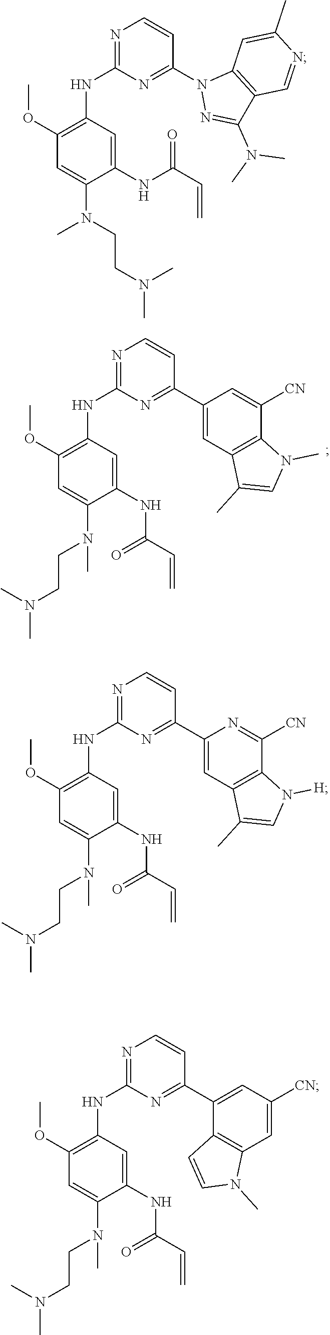

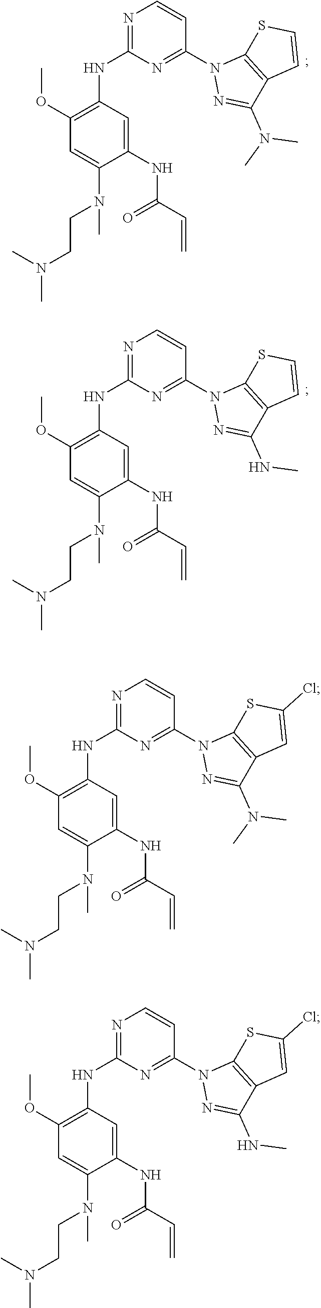 [SMILES: C=CC(=O)Nc1cc(Nc2nccc(-n3nc(N(C)C)c4ccsc43)n2)c(OC)cc1N(C)CCN(C)C ; C=CC(=O)Nc1cc(Nc2nccc(-n3nc(NC)c4ccsc43)n2)c(OC)cc1N(C)CCN(C)C ; C=CC(=O)Nc1cc(Nc2nccc(-n3nc(N(C)C)c4cc(Cl)sc43)n2)c(OC)cc1N(C)CCN(C)C ; C=CC(=O)Nc1cc(Nc2nccc(-n3nc(NC)c4cc(Cl)sc43)n2)c(OC)cc1N(C)CCN(C)C]

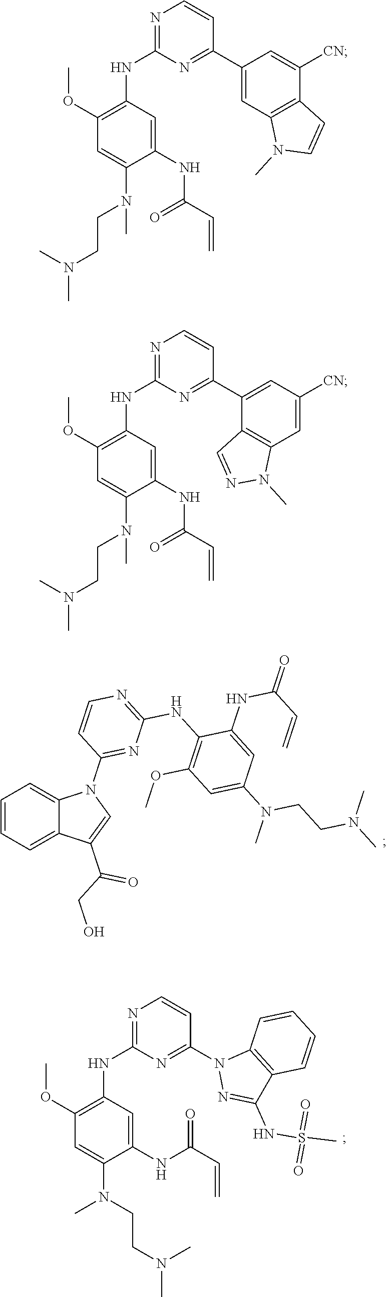 [SMILES: C=CC(=O)Nc1cc(Nc2nccc(-c3cc(C#N)c4ccn(C)c4c3)n2)c(OC)cc1N(C)CCN(C)C ; C=CC(=O)Nc1cc(Nc2nccc(-c3cc(C#N)cc4c3cnn4C)n2)c(OC)cc1N(C)CCN(C)C ; C=CC(=O)Nc1cc(N(C)CCN(C)C)cc(OC)c1Nc1nccc(-n2cc(C(=O)CO)c3ccccc32)n1 ; C=CC(=O)Nc1cc(Nc2nccc(-n3nc(NS(C)(=O)=O)c4ccccc43)n2)c(OC)cc1N(C)CCN(C)C]

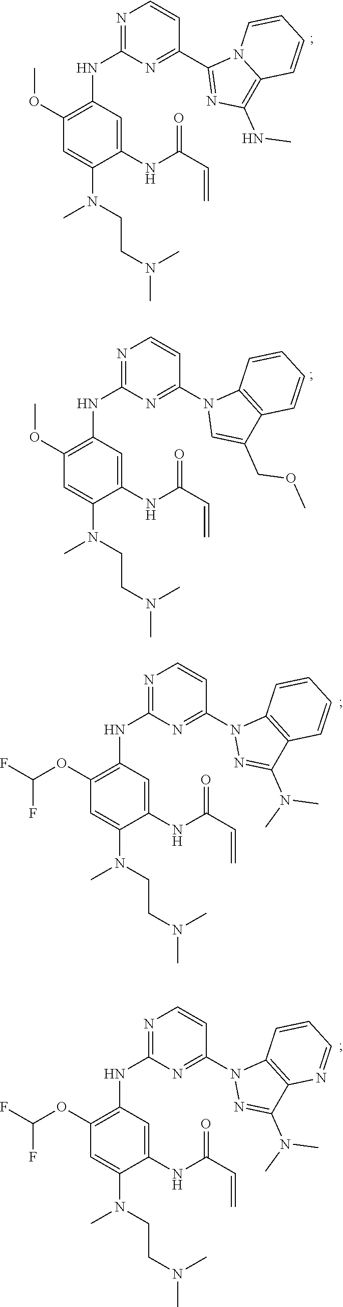

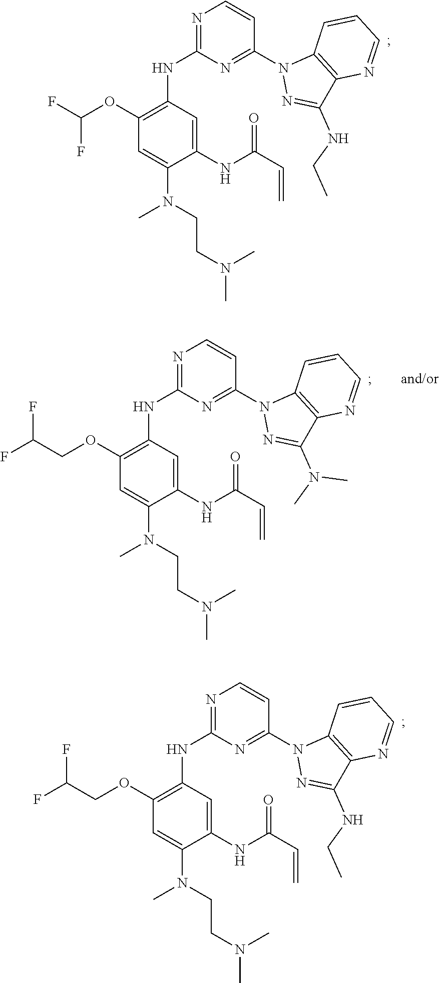

or pharmaceutically acceptable salt thereof.

In one embodiment, the present disclosure relates to one or more of the following compounds selected from:

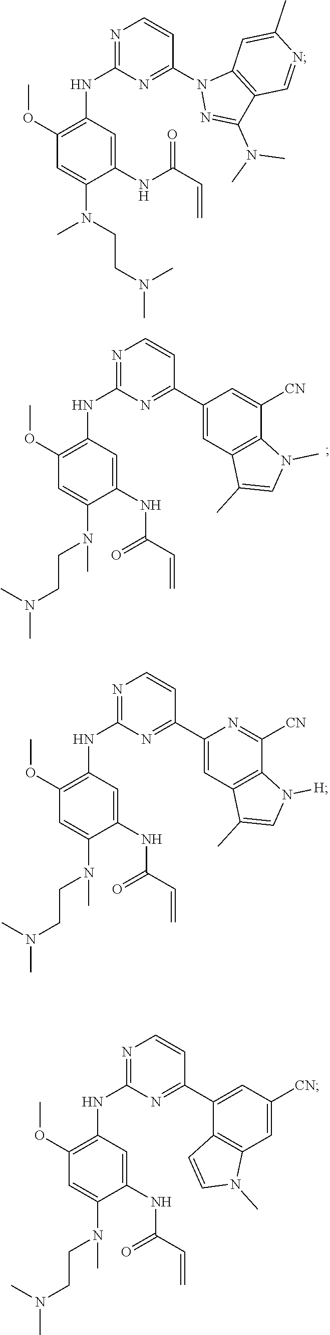

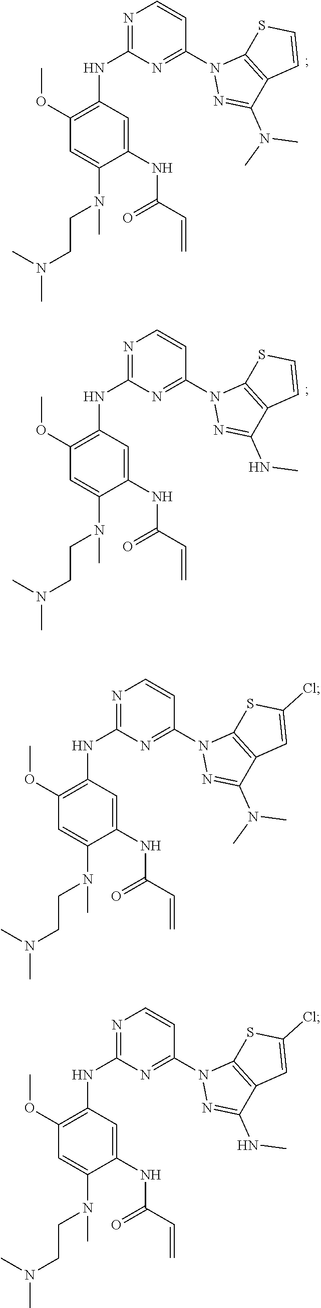 [SMILES: C=CC(=O)Nc1cc(Nc2nccc(-n3nc(N(C)C)c4ccsc43)n2)c(OC)cc1N(C)CCN(C)C ; C=CC(=O)Nc1cc(Nc2nccc(-n3nc(NC)c4ccsc43)n2)c(OC)cc1N(C)CCN(C)C ; C=CC(=O)Nc1cc(Nc2nccc(-n3nc(N(C)C)c4cc(Cl)sc43)n2)c(OC)cc1N(C)CCN(C)C ; C=CC(=O)Nc1cc(Nc2nccc(-n3nc(NC)c4cc(Cl)sc43)n2)c(OC)cc1N(C)CCN(C)C]

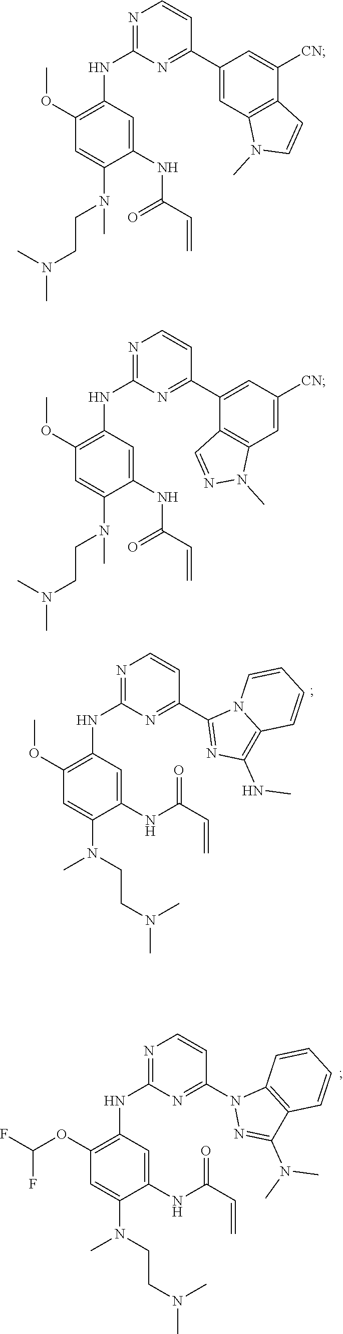 [SMILES: C=CC(=O)Nc1cc(Nc2nccc(-c3cc(C#N)c4ccn(C)c4c3)n2)c(OC)cc1N(C)CCN(C)C ; C=CC(=O)Nc1cc(Nc2nccc(-c3cc(C#N)cc4c3cnn4C)n2)c(OC)cc1N(C)CCN(C)C ; C=CC(=O)Nc1cc(Nc2nccc(-c3nc(NC)c4ccccn34)n2)c(OC)cc1N(C)CCN(C)C ; C=CC(=O)Nc1cc(Nc2nccc(-n3nc(N(C)C)c4ccccc43)n2)c(OC(F)F)cc1N(C)CCN(C)C]

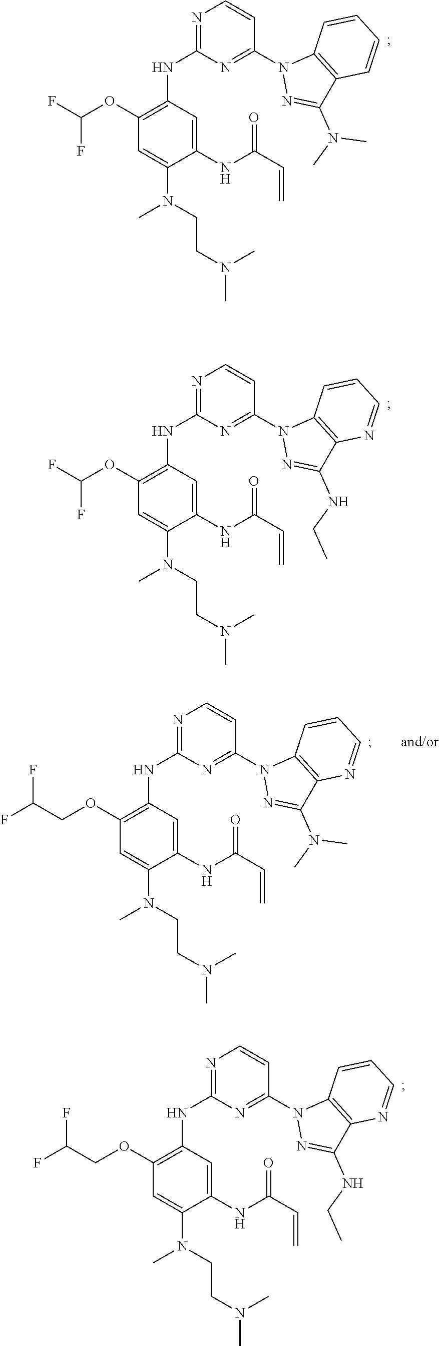

or pharmaceutically acceptable salt thereof.

In one embodiment, the present disclosure relates to one or more of the following compounds selected from:

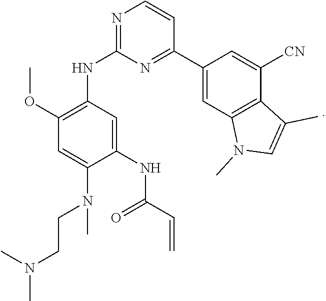

In one embodiment, the present disclosure relates to one or more of the following compounds selected from:

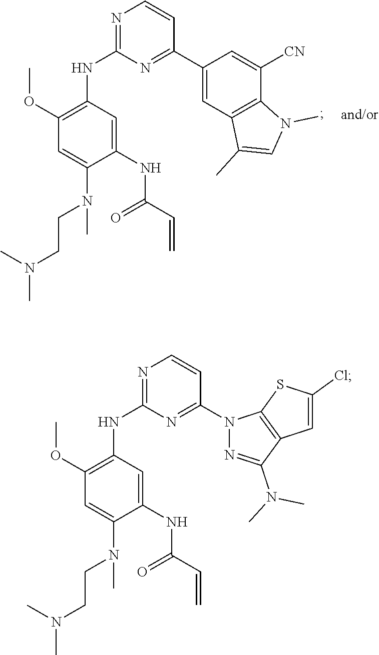

or pharmaceutically acceptable salt thereof.

In another embodiment, the present disclosure relates to one or more of the following compounds selected from:

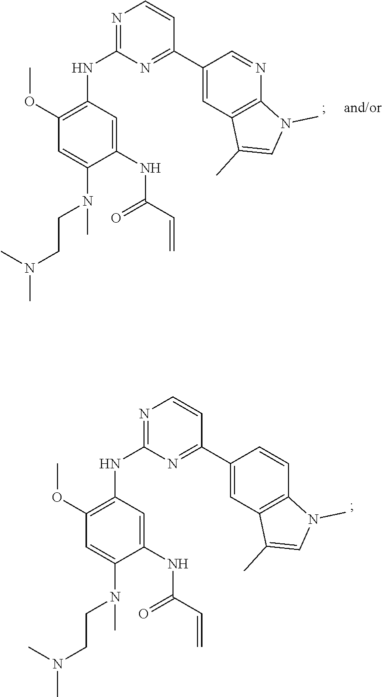

or pharmaceutically acceptable salt thereof.

In one embodiment of formula (I), (A), (B), (C), (C-I), (D), (D-I), (E), (E-I), (F), (G), (H), (H-I), (J), (K), (L), (M), (N), (O), and/or (P), R¹⁰ is not H. In one embodiment of formula (I), (A), (B), (C), (C-I), (D), (D-I), (E), (E-I), (F), (G), (H), (H-I), (J), (K), (L), (M), (N), (O), and/or (P), R¹⁰ is —CD₃ or C₁-C₆ alkyl. In one embodiment of formula (I), (A), (B), (C), (C-I), (D), (D-I), (E), (E-I), (F), (G), (H), (H-I), (J), (K), (L), (M), (N), (O), and/or (P), R¹⁰ is —CD₃, —CH₃, —CH₂CH₃, or —CH(CH₃)₂.

In one embodiment of formula (I), (A), (B), (C), (C-I), (D), (D-I), (E), (E-I), (F), (G), (H), (H-I), (J), (K), (L), (M), (N), (O), and/or (P), the compound can be in a form of an N-oxide.

In one embodiment, the compounds of the invention exclude the compounds exemplified in CN 105085489 A, WO 2015/127872, WO2013/014448, CN 105001208 A, CN 104844580 A, WO 2015/175632, WO 2015/188777, WO 2016/105525, WO2016060443, WO 2016/029839, WO 2016/054987, WO 2016/015453, WO 2016/070816, and/or WO 2015/195228.

In one embodiment, the compounds of the invention exclude the compounds exemplified in CN 104761585 A and/or CN 104761544 A.

In one embodiment of formula (A) or any subgenera of formula (I) thereof; R^{4a}, R^{4b}, R^{4c}, and R^{4d} etc are embodiments of R⁴.

Compounds of the present disclosure may also exist in several tautomeric forms, and the depiction herein of one tautomer is for convenience only, and is also understood to encompass other tautomers of the form shown. Accordingly, the chemical structures depicted herein encompass all possible tautomeric forms of the illustrated compounds. The term “tautomer” as used herein refers to isomers that change into one another with great ease so that they can exist together in equilibrium. For example, ketone and enol are two tautomeric forms of one compound. In another example, a substituted 1,2,4-triazole derivative may exist in at least three tautomeric forms as shown below:

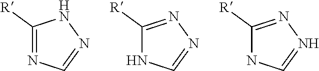

R′ is an optionally substituted alkyl.

One skilled in the art will recognize that substituents, variables, and other moieties of the compounds of Formula (I), (A), (B), (C), (C-I), (D), (D-I), (E), (E-I), (F), (G), (H), (H-I), (J), (K), (L), (M), (N), (O), and/or (P), or subgeneric structures or species thereof, should be selected in order to provide a compound which is sufficiently stable to provide a pharmaceutically useful compound which can be formulated into an acceptably stable pharmaceutical composition. Furthermore, one skilled in the art will recognize that substituents, variables, and other moieties of the compounds of Formula (I), (A), (B), (C), (C-I), (D), (D-I), (E), (E-I), (F), (G), (H), (H-I), (J), (K), (L), (M), (N), (O), and/or (P) subgeneric structures or species thereof, should be selected as such that it would not yield any compound which has structural feature in violation of the basic principles of the chemistry art. For example, in one embodiment of Formula (I), or subgeneric structures or species thereof, two bonds of a, b, c, d, and e are (formal) double bonds and the remaining ones are (formal) single bonds, such that none of the atoms X¹, X², X³, X⁴, and X⁵ has two double bonds attached thereto. In another embodiment of Formula (I), wherein A¹, A² and A³, when X¹ is N, X² is C═O, C═NR¹⁰ or C═S, X³ is O, S or NR¹⁰ and X⁴ and X⁵ are C, then only e is a formal double bond. In another embodiment of Formula (I), wherein A¹ and A³, when X¹ and one of X⁴ and X⁵ are N, then only b will be a (formal) double bond. In another embodiment of Formula (I), wherein A³, when X¹ is C, and X³ is O, S or NR¹⁰, and one of X⁴ and X⁵ is N, then only c will be a (formal) double bond.

In one embodiment, the present disclosure relates to one or more of the compounds disclosed in Examples 1-30.

In one embodiment, the compounds of the invention include, but are not limited to:

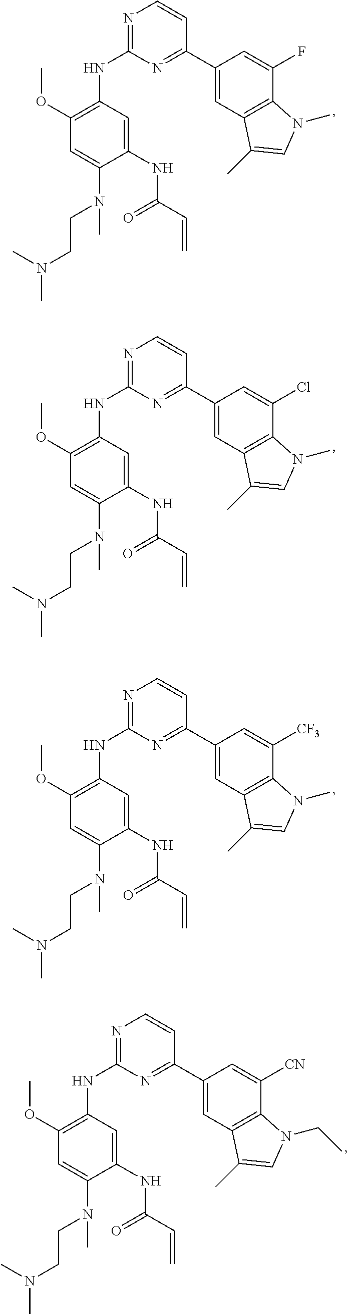

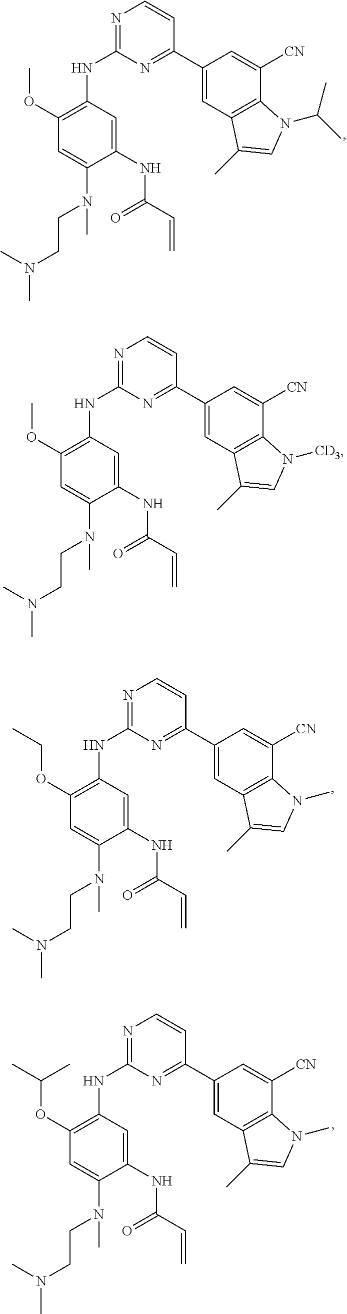

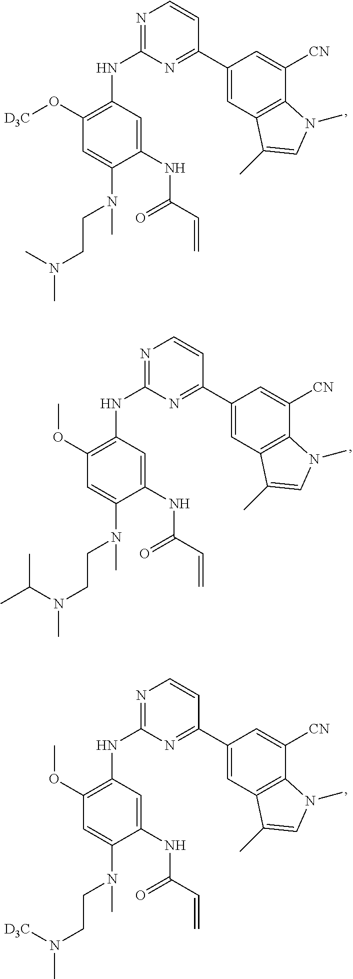

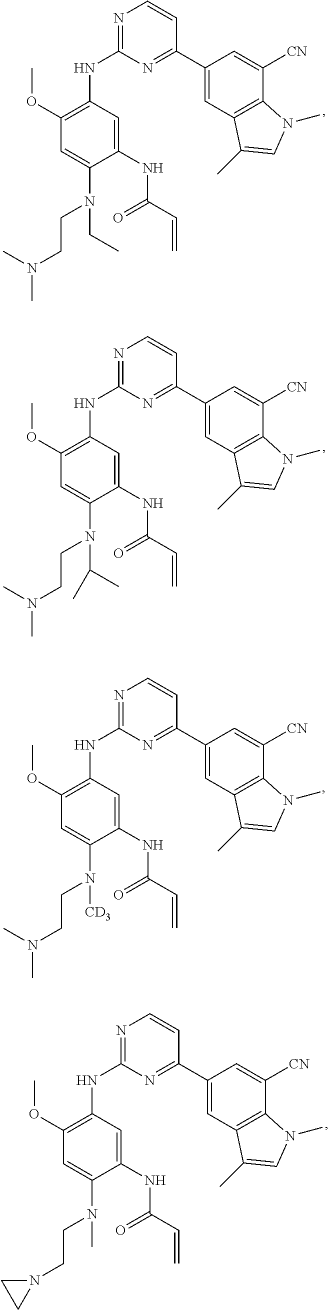

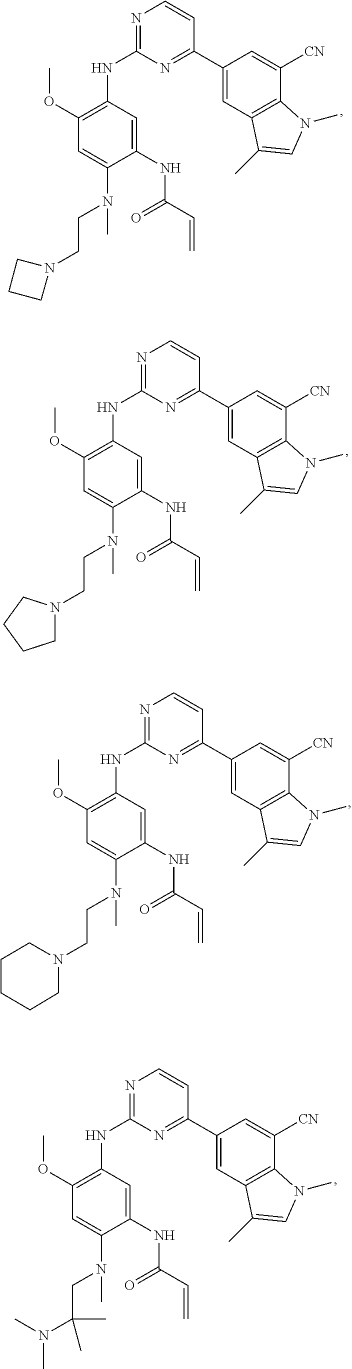

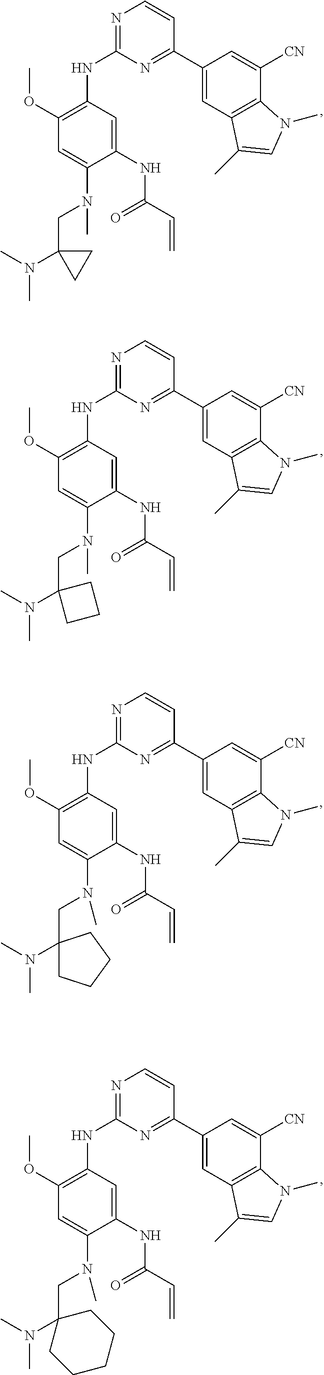

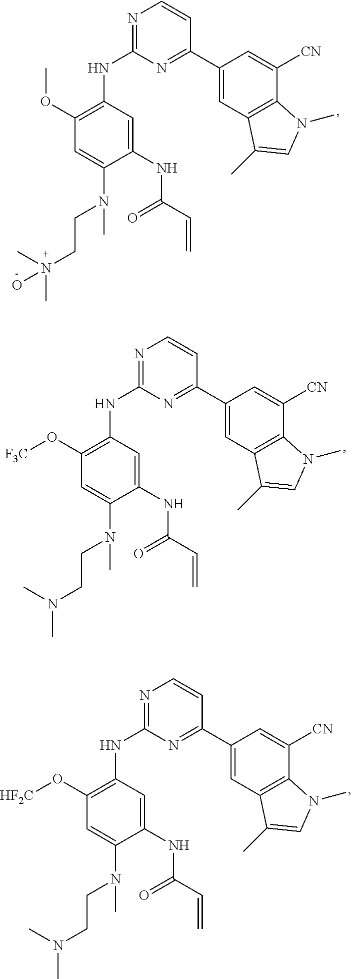

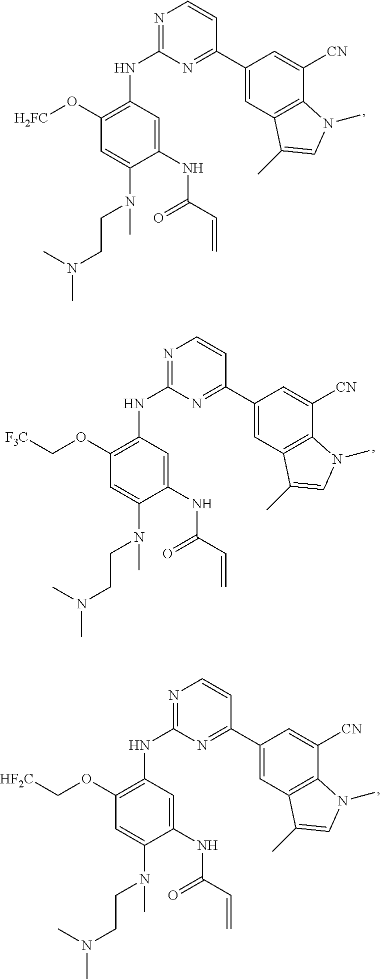

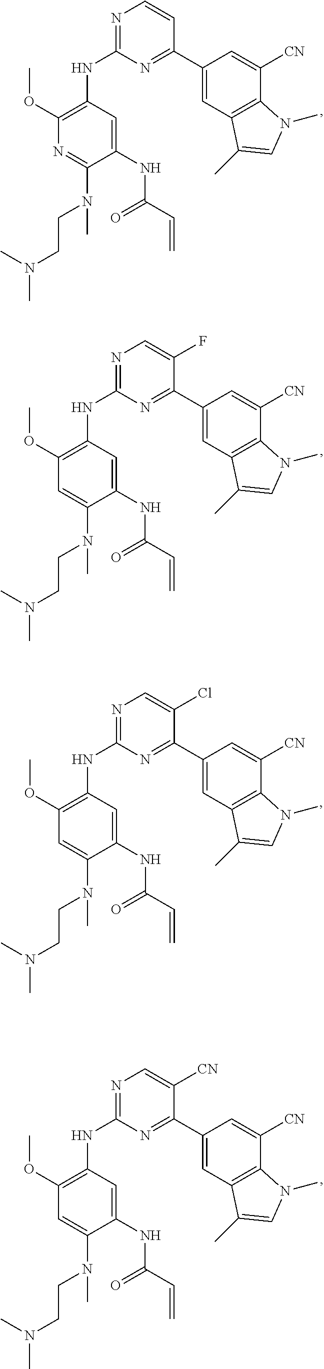

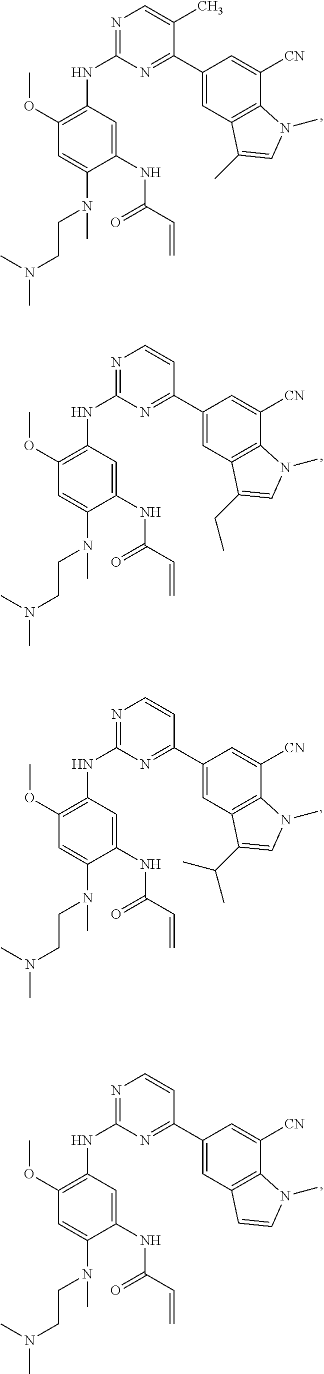

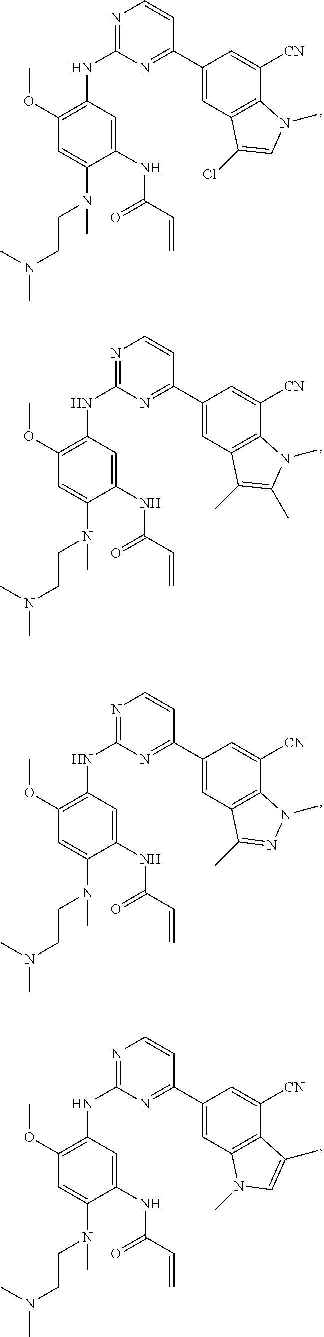

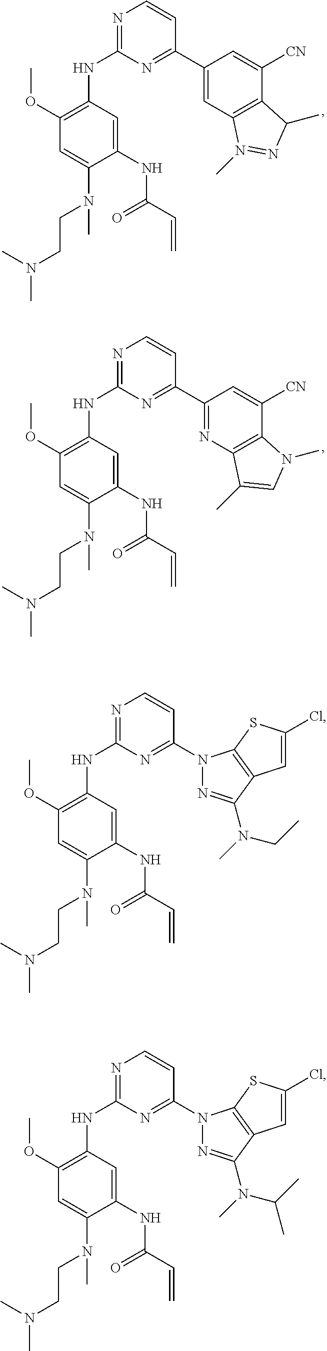

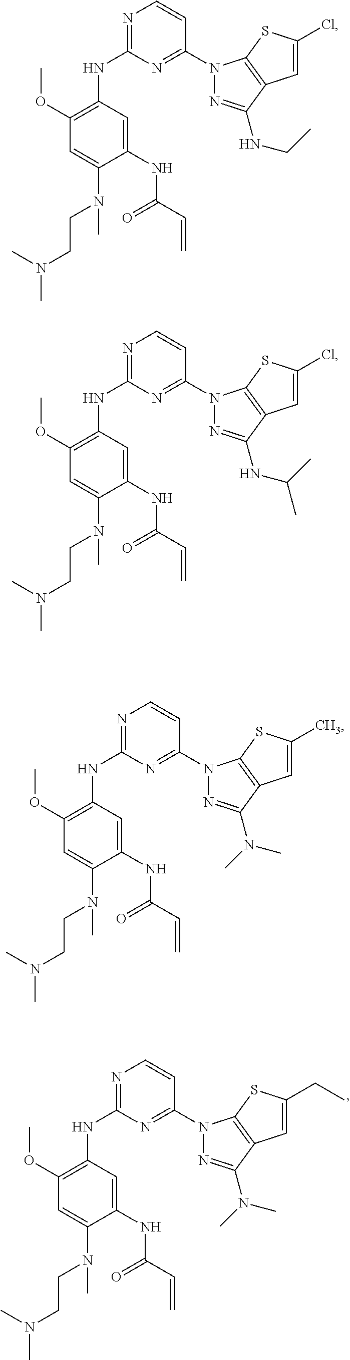 [SMILES: C=CC(=O)Nc1cc(Nc2nccc(-n3nc(NCC)c4cc(Cl)sc43)n2)c(OC)cc1N(C)CCN(C)C ; C=CC(=O)Nc1cc(Nc2nccc(-n3nc(NC(C)C)c4cc(Cl)sc43)n2)c(OC)cc1N(C)CCN(C)C ; C=CC(=O)Nc1cc(Nc2nccc(-n3nc(N(C)C)c4cc(C)sc43)n2)c(OC)cc1N(C)CCN(C)C ; C=CC(=O)Nc1cc(Nc2nccc(-n3nc(N(C)C)c4cc(CC)sc43)n2)c(OC)cc1N(C)CCN(C)C]

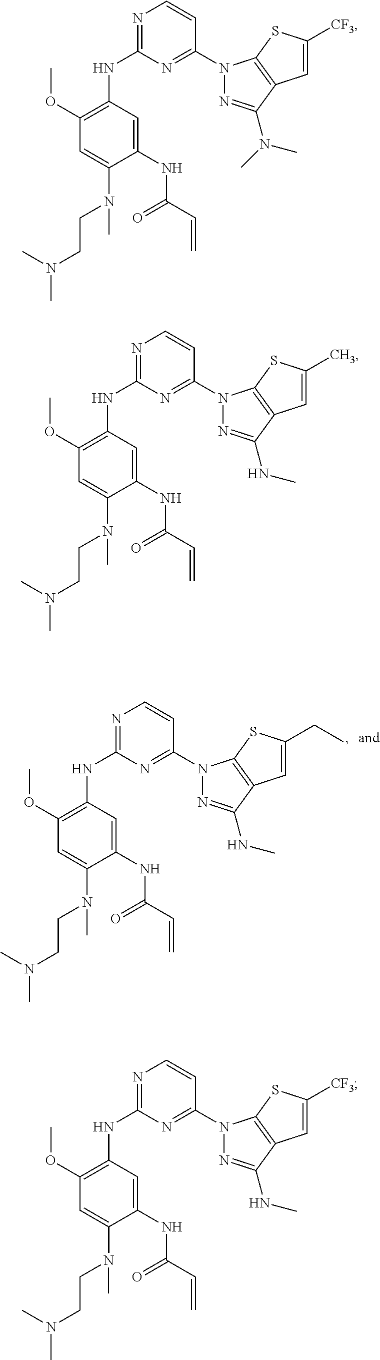

pharmaceutically acceptable salt thereof.

For EGFR target, the compounds listed above would show similar activity and selectivity profile compared to the compounds listed as Examples.

### Pharmaceutical Compositions

In one embodiment, the present disclosure relates to a pharmaceutical composition comprising a compound of the invention, or a pharmaceutically acceptable salt, solvate, ester, or prodrug thereof, and a pharmaceutically acceptable carrier.

As used in the preparations and examples the following terms have the indicated meanings; “ng” refers to nanograms; “g” refers to micrograms; “mg” refers to milligrams; “g” refers to grams; “kg” refers to kilograms; “nmole” or “nmol” refers to nanomoles; “mmol” refers to millimoles; “mol” refers to moles; “M” refers to molar, “mM” refers to millimolar, “M” refers to micromolar, “nM” refers to nanomolar, “L” refers to liters, “mL” refers to milliliters, “μL” refers to microliters.

Pharmaceutically acceptable salts of the compounds of the invention include the acid addition and base salts (including disalts) thereof.

Suitable acid addition salts are formed from acids which form non-toxic salts. Examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts.

For a review on suitable salts, see “Handbook of Pharmaceutical Salts: Properties, Selection, and Use” by Stahl and Wermuth (Wiley-VCH, Weinheim, Germany, 2002).

A pharmaceutically acceptable salt of a compound of the invention may be readily prepared by mixing together solutions of the compound and the desired acid or base, as appropriate. The salt may precipitate from solution and be collected by filtration or may be recovered by evaporation of the solvent. The degree of ionization in the salt may vary from completely ionized to almost non-ionized.

Compounds of the invention containing one or more asymmetric carbon atoms can exist as two or more stereoisomers. Where a compound of the invention contains an alkenyl or alkenylene group, geometric cis/trans (or Z/E) isomers are possible. Where the compound contains, for example, a keto or oxime group or an aromatic moiety, tautomeric isomerism (‘tautomerism’) can occur. It follows that a single compound may exhibit more than one type of isomerism.

Included within the scope of the claimed compounds of the present invention are all stereoisomers, geometric isomers and tautomeric forms of the compounds of the invention, including compounds exhibiting more than one type of isomerism, and mixtures of one or more thereof. Also included are acid addition or base salts wherein the counterion is optically active, for example, D-lactate or L-lysine, or racemic, for example, DL-tartrate or DL-arginine.

Cis/trans isomers may be separated by conventional techniques well known to those skilled in the art, for example, chromatography and fractional crystallization.

Compounds of the current invention may also exhibit atropisomerism, where restricted rotation, especially around the bond joining two aryl rings in a biaryl, causes different rotational isomers to be not interconvertible at normal ambient temperatures, and quite possibly not at temperatures where the molecule as a whole remains thermally stable. In such cases distinct stereoisomers due to atropisomerism are also claimed.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC), especially in a simulated moving bed (SMB) configuration.

Alternatively, the racemate (or a racemic precursor) may be reacted with a suitable optically active compound, for example, an alcohol, or, in the case where the compound of the invention contains an acidic or basic moiety, an acid or base such as tartaric acid or 1-phenylethylamine. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% isopropanol, typically from 2 to 20%, and from 0 to 5% of an alkylamine, typically 0.1% diethylamine. Concentration of the eluate affords the enriched mixture.

Mixtures of stereoisomers may be separated by conventional techniques known to those skilled in the art. [see, for example, “Stereochemistry of Organic Compounds” by E L Eliel (Wiley, New York, 1994).]

The present invention includes all pharmaceutically acceptable isotopically labeled compounds of the invention wherein one or more atoms are replaced by atoms having the same atomic number, but an atomic mass or mass number different from the atomic mass or mass number usually found in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulfur, such as ³⁵S.

Certain isotopically-labelled compounds of the invention, for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. 2H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labeled compounds of the invention can generally be prepared by conventional techniques known to those skilled in the art or by processes analogous to those described in the accompanying Examples and Preparations using an appropriate isotopically-labeled reagents in place of the non-labeled reagent previously employed.

The compounds of the present invention may be administered as prodrugs. Thus certain derivatives of compounds of the invention which may have little or no pharmacological activity themselves can, when administered into or onto the body, be converted into compounds of formula 1 (or other formulae disclosed herein) having the desired activity, for example, by hydrolytic cleavage. Such derivatives are referred to as ‘prodrugs’. Further information on the use of prodrugs may be found in ‘Pro-drugs as Novel Delivery Systems, Vol. 14, ACS Symposium Series (T Higuchi and W Stella) and ‘Bioreversible Carriers in Drug Design’, Pergamon Press, 1987 (ed. E B Roche, American Pharmaceutical Association).

Prodrugs can, for example, be produced by replacing appropriate functionalities present in the compounds of the invention with certain moieties known to those skilled in the art as ‘pro-moieties’ as described, for example, in “Design of Prodrugs” by H Bundgaard (Elsevier, 1985).

Some examples of such prodrugs include:
where the compound contains a carboxylic acid functionality (—COOH), an ester thereof, for example, replacement of the hydrogen with C1-C6 alkyl;
where the compound contains an alcohol functionality (—OH), an ether thereof, for example, replacement of the hydrogen with C₁-C₆ alkanoyloxymethyl (—C1-C₆ acyloxymethyl); and
where the compound contains a primary or secondary amino functionality (—NH₂ or —NHR where R is not H), an amide thereof, for example, replacement of one or both hydrogens with (C₁-C₁₀)alkanoyl (—C₁-C₁₀ acyl).

Further examples of replacement groups in accordance with the foregoing examples and examples of other prodrug types may be found in the aforementioned references.

Finally, certain compounds of the invention may themselves act as prodrugs of other compounds of the invention.

### Methods of Treatment

In one embodiment, the present invention relates to a method useful for treating cancer selected from lung cancer, colorectal cancer, pancreatic cancer, head and neck cancers, breast cancer, ovarian cancer, uterine cancer, liver cancer, and stomach cancer. In another embodiment, the cancer is non-small cell lung cancer (NSCLC).

In one embodiment, the method disclosed herein relates to treatment of cancer, wherein the cancer results from a mutation in the exon 20 domain of EGFR. In some embodiments, the mutation in the exon 20 domain of EGFR is selected from NPG, ASV, or T790M. In one embodiment, the mutation in the exon 20 domain of EGFR is T790M concurrent with an exon 19 insertion mutation or an exon 21 point mutation.

In one embodiment, the method of treatment of cancer is particularly useful for patient who is resistant to a kinase inhibitor other that a compound of the invention, or a pharmaceutically acceptable salt, solvate, ester, or prodrug thereof. In another embodiment, the kinase inhibitor is an EGFR inhibitor.

The invention also relates to a method for inhibiting EGFR, or a mutation thereof, in a patient in need thereof, comprising administering to the patient a therapeutically effective amount of a compound of the invention, or a pharmaceutically acceptable salt, solvate, ester, or prodrug thereof. In one embodiment, the mutation is in the exon 20 domain of EGFR.

The invention further relates to therapeutic methods and uses comprising administering the compounds of the invention, or pharmaceutically acceptable salts thereof, alone or in combination with other therapeutic or palliative agents.

In one embodiment, the invention relates to a method for treating or inhibiting cell proliferation, cell invasiveness, metastases, apoptosis, or angiogenesis in a mammal comprising administering to the mammal a therapeutically effective amount of a compound of the invention, or pharmaceutically acceptable salt thereof.

In another embodiment, the invention relates to a method for treating or inhibiting cell proliferation, cell invasiveness, metastases, apoptosis, or angiogenesis in a mammal comprising administering to the mammal a therapeutically effective amount of a compound of the invention, or pharmaceutically acceptable salt thereof, in combination with a with a second therapeutic agent wherein the amounts of the compound of the invention and the second therapeutic agent together are effective in treating or inhibiting said cell proliferation, cell invasiveness, metastases, apoptosis, or angiogenesis.

In one embodiment, the second therapeutic agent is an anti-tumor agent which is selected from the group consisting of mitotic inhibitors, alkylating agents, antimetabolites, intercalating antibiotics, growth factor inhibitors, radiation, cell cycle inhibitors, enzymes, topoisomerase inhibitors, biological response modifiers, antibodies, cytotoxics, anti-hormones, and anti-androgens.

In other embodiments, the cell proliferation, cell invasiveness, metastases, apoptosis, or angiogenesis is mediated by members of the erbB family of RTKs, mainly EGFR, and most probably T790M mutant forms of EGFR.

In a further embodiment, the cell proliferation, cell invasiveness, metastases, apoptosis, or angiogenesis is associated with a cancer selected from the group consisting of glioblastoma, lung cancer (e.g., squamous cell carcinoma, non-small cell lung cancer, adenocarcinoma, bronchioloalveolar carcinoma (BAC), BAC with focal invasion, adenocarcinoma with BAC features, and large cell carcinoma), pancreatic cancer, head and neck cancers (e.g., squamous cell carcinoma), breast cancer, colorectal cancer, epithelial cancer (e.g., squamous cell carcinoma), ovarian cancer, and prostate cancer, and any other cancer which overexpresses members of the erbB family, or which contains oncogenicall activating mutants of the erbB family, regardless of whether those proteins are overexpressed in the tumor.

A further embodiment of the invention relates to a compound of the invention for use as a medicament, and in particular for use in the treatment of diseases where the inhibition of EGFR and/or a mutant EGFR protein, e.g., L858R/T790M EGFR, activity may induce benefit, such as cancer. A still further embodiment of the present invention relates to the use of the compounds of the invention, or pharmaceutically acceptable salts thereof, for the manufacture of a drug having an EGFR inhibitory activity for the treatment of EGFR mediated diseases and/or conditions, in particular the diseases and/or conditions listed above.

The term “therapeutically effective amount” refers to that amount of a compound being administered which will relieve to some extent one or more of the symptoms of the disorder being treated. Regarding the treatment of cancer, a therapeutically effective amount refers to that amount which has the effect of reducing the size of the tumor, inhibiting (i.e., slowing or stopping) tumor metastases, inhibiting (i.e. slowing or stopping) tumor growth or tumor invasiveness, and/or relieving to some extent one or more signs or symptoms related to the cancer.

A therapeutically effective amount can be readily determined by the attending diagnostician, as one skilled in the art, by the use of conventional techniques and by observing results obtained under analogous circumstances. In determining the therapeutically effective amount, the dose, a number of factors are considered by the attending diagnostician, including, but not limited to: the species of mammal; its size, age, and general health; the specific disease involved; the degree of involvement or the severity of the disease; the response of the individual patient; the particular compound administered; the mode of administration; the bioavailability characteristic of the preparation administered; the dose regimen selected; the use of concomitant medication; and other relevant circumstances.

The term “treating”, as used herein, unless otherwise indicated, means reversing, alleviating, inhibiting the progress of, or preventing the disorder or condition to which such term applies, or one or more symptoms of such disorder or condition. The term “treatment” also refers to the act of treating as “treating” is defined immediately above. The term “treating” also includes adjuvant treatment of a mammal.

As used herein “cancer” refers to any malignant and/or invasive growth or tumor caused by abnormal cell growth, including solid tumors named for the type of cells that form them, cancer of blood, bone marrow, or the lymphatic system. Examples of solid tumors include but not limited to sarcomas and carcinomas. Examples of cancers of the blood include but not limited to leukemias, lymphomas and myeloma. The term “cancer” includes but is not limited to a primary cancer that originates at a specific site in the body, a metastatic cancer that has spread from the place in which it started to other parts of the body, a recurrence from the original primary cancer after remission, and a second primary cancer that is a new primary cancer in a person with a history of previous cancer of a different type.

In another embodiment, the invention provides a method for inhibiting cell proliferation, comprising contacting cells with a compound of the invention or a pharmaceutically acceptable salt thereof in an amount effective to inhibit proliferation of the cells. In another embodiment, the invention provides methods for inducing cell apoptosis, comprising contacting cells with a compound described herein in an amount effective to induce apoptosis of the cells.

“Contacting” refers to bringing a compound or pharmaceutically acceptable salt of the invention and a cell expressing mutant EGFR or one of the other target kinases which is playing a transforming role in the particular cell type, together in such a manner that the compound can affect the activity of EGFR, or the other kinase, either directly or indirectly. Contacting can be accomplished in vitro (i.e., in an artificial environment such as, e.g., without limitation, in a test tube or culture medium) or in vivo (i.e., within a living organism such as, without limitation, a mouse, rat or rabbit.)

In some embodiments, the cells are in a cell line, such as a cancer cell line. In other embodiments, the cells are in a tissue or tumor, and the tissue or tumor may be in a mammal, including a human.

Administration of the compounds of the invention may be effected by any method that enables delivery of the compounds to the site of action. These methods include oral routes, intraduodenal routes, parenteral injection (including intravenous, subcutaneous, intramuscular, intravascular or infusion), topical, and rectal administration.

Dosage regimens may be adjusted to provide the optimum desired response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage.

Dosage unit form, as used herein, refers to physically discrete units suited as unitary dosages for the mammalian mammals to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the chemotherapeutic agent and the particular therapeutic or prophylactic effect to be achieved, and (b) the limitations inherent in the art of compounding such an active compound for the treatment of sensitivity in individuals.

Appropriate dosages may vary with the type and severity of the condition to be treated and may include single or multiple doses. An attending diagnostician understands that for any particular mammal, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present invention encompasses intra-patient dose-escalation as determined by the skilled artisan. Determining appropriate dosages and regimens for administration of the chemotherapeutic agent are well-known in the relevant art and would be understood to be encompassed by the skilled artisan once provided the teachings disclosed herein.

Useful dosages of the compounds of the invention can be determined by comparing their in vitro activity, and in vivo activity in animal models. The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

The compounds of the present invention can be administered to a patient at dosage levels in the range of about 0.1 to about 2,000 mg per day. For a normal human adult having a body weight of about 70 kilograms, a dosage in the range of about 0.01 to about 10 mg per kilogram of body weight per day is preferable. However, the specific dosage used can vary. For example, the dosage can depended on a numbers of factors including the requirements of the patient, the severity of the condition being treated, and the pharmacological activity of the compound being used. The determination of optimum dosages for a particular patient is well-known to those skilled in the art. In some instances, dosage levels below the lower limit of the aforesaid range may be more than adequate, while in other cases still larger doses may be employed without causing harmful side effect, provided that such larger doses are first divided into several smaller doses for administration throughout the day.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Pharmaceutical compositions suitable for the delivery of compounds of the invention and methods for their preparation will be readily apparent to those skilled in the art. Such compositions and methods for their preparation can be found, for example, in ‘Remington's Pharmaceutical Sciences’, 19th Edition (Mack Publishing Company, 1995), the disclosure of which is incorporated herein by reference in its entirety.

The compounds of the invention may be administered orally. Oral administration may involve swallowing, so that the compound enters the gastrointestinal tract, or buccal or sublingual administration may be employed by which the compound enters the blood stream directly from the mouth. Formulations suitable for oral administration include solid formulations such as tablets, capsules containing particulates, liquids, or powders, lozenges (including liquid-filled), chews, multi- and nano-particulates, gels, solid solution, liposome, films (including muco-adhesive), ovules, sprays and liquid formulations.

Liquid formulations include suspensions, solutions, syrups and elixirs. Such formulations may be used as fillers in soft or hard capsules and typically include a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid.

The compounds of the invention may also be used in fast-dissolving, fast-disintegrating dosage forms such as those described in Expert Opinion in Therapeutic Patents, 11 (6), 981986 by Liang and Chen (2001), the disclosure of which is incorporated herein by reference in its entirety.

For tablet dosage forms, depending on dose, the drug may make up from 1 wt % to 80 wt % of the dosage form, more typically from 5 wt % to 60 wt % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinized starch and sodium alginate. Generally, the disintegrant will comprise from 1 wt % to 25 wt %, preferably from 5 wt % to 20 wt % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinized starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet.

Other possible ingredients include anti-oxidants, colorants, flavoring agents, preservatives and taste-masking agents.

Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated.

The formulation of tablets is discussed in “Pharmaceutical Dosage Forms: Tablets, Vol. 1”, by H. Lieberman and L. Lachman, Marcel Dekker, N.Y., N.Y., 1980 (ISBN 0-8247-6918-X).

The foregoing formulations for the various types of administration discussed above may be formulated to be immediate and/or modified release. Modified release formulations include delayed-, sustained-, pulsed-, controlled-, targeted and programmed release. Suitable modified release formulations for the purposes of the invention are described in U.S. Pat. No. 6,106,864.

Details of other suitable release technologies such as high energy dispersions and osmotic and coated particles are to be found in Verma et al, Pharmaceutical Technology On-line, 25(2), 1-14 (2001). The use of chewing gum to achieve controlled release is described in WO 00/35298.

The compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of compounds of the invention used in the preparation of parenteral solutions may be increased by the use of appropriate formulation techniques, such as the incorporation of solubility-enhancing agents.

Formulations for parenteral administration may be formulated to be immediate and/or modified release. Thus, compounds of the invention may be formulated as a solid, semi-solid, or thixotropic liquid for administration as an implanted depot providing modified release of the active compound. Examples of such formulations include drug-coated stents and poly(glycolide-co-dl-lactide) or PGLA microspheres.

The compounds of the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration. Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

The term “combination therapy” refers to the administration of a compound of the invention together with at least one additional pharmaceutical or medicinal agent, either sequentially or simultaneously. Combination therapy encompasses the use of the compounds of the present invention and other therapeutic agents either in discreet dosage forms or in the same pharmaceutical formulation. The compounds of the invention may be used in combination (administered simultaneously, sequentially, or separately) with one or more therapeutic agents.

In one embodiment of the present invention the anti-cancer agent used in conjunction with a compound of the invention and pharmaceutical compositions described herein is an antiangiogenesis agent (e.g., an agent that stops tumors from developing new blood vessels). Examples of anti-angiogenesis agents include for example VEGF inhibitors, VEGFR inhibitors, TIE-2 inhibitors, PDGFR inhibitors, angiopoetin inhibitors, PKCI3 inhibitors, CQX-2 (cyclooxygenase II) inhibitors, integrins (alpha-v/beta-3), MMP-2 (matrix-metalloprotienase 2) inhibitors, and MMP-9 (matrixmetalloprotienase 9) inhibitors. Preferred anti-angiogenesis agents include sunitinib (SutenFM), bevacizumab (Avastin™), and axitinib (AG 13736).

Additional anti-angiogenesis agents include vatalanib (CGP 79787), Sorafenib (Nexavar™), pegaptanib octasodium (Macugen™), vandetanib (Zactima™), PF-0337210 (Pfizer), SU 14843 (Pfizer), AZD 2171 (AstraZeneca), ranibizumab (Lucentis™), Neovastat™ (AE 941), tetrathiomolybdata (Coprexa™), AMG 706 (Amgen), VEGF Trap (AVE 0005), CEP 7055 (Sanofi-Aventis), XL 880 (Exelixis), telatinib (BAY 57-9352), and CP-868,596 (Pfizer).

Other examples of anti-angiogenesis agents which can be used in conjunction with a compound of the invention and pharmaceutical compositions described herein include celecoxib (Celebrex™), parecoxib (Dynastat™), deracoxib (SC 59046), lumiracoxib (Preige™), valdecoxib (Bextra™), rofecoxib (Vioxx™), iguratimod (Careram™), IP 751 (Invedus), SC-58125 (Pharmacia) and etoricoxib (Arcoxia™). Other anti-angiogenesis agents include exisulind (Aptosyn™), salsalate (Amigesic™), diflunisal (Dolobid™), ibuprofen (Motrin™), ketoprofen (Orudis™), nabumetone (Relafen™), piroxicam (Feldene™), naproxen (AIeve™, Naprosyn™), diclofenac (Voltaren™), indomethacin (Indocin™), sulindac (ClinoriI™), tolmetin (Tolectin™), etodolac (Lodine™), ketorolac (Toradol™), and oxaprozin (Daypro™). Other anti-angiogenesis agents include ABT 510 (Abbott), apratastat (TMI 005), AZD 8955 (AstraZeneca), incyclinide (Metastat™), and PCK 3145 (Procyon). Other antiangiogenesis agents include acitretin (Neotigason™), plitidepsin (Aplidine™), cilengtide (EMD 121974), combretastatin A4 (CA4P), fenretinide (4 HPR), halofuginone (Tempostatin™), Panzem™ (2-methoxyestradiol), PF-03446962 (Pfizer), rebimastat (BMS 275291), catumaxomab (Removab™), lenalidomide (Revlimid™), squalamine (EVIZON™), thalidomide (Thalomid™), Ukrain™ (NSC 631570), Vitaxin™ (MEDI 522), and zoledronic acid (Zometa™).

In another embodiment the anti-cancer agent is a so called signal transduction inhibitor (e.g., inhibiting the means by which regulatory molecules that govern the fundamental processes of cell growth, differentiation, and survival communicated within the cell). Signal transduction inhibitors include small molecules, antibodies, and antisense molecules. Signal transduction inhibitors include for example kinase inhibitors (e.g., tyrosine kinase inhibitors or serine/threonine kinase inhibitors) and cell cycle inhibitors. More specifically signal transduction inhibitors include, for example, farnesyl protein transferase inhibitors, EGF inhibitor, ErbB-1 (EGFR), ErbB-2, pan erb, IGF1 R inhibitors, MEK, c-Kit inhibitors, FLT-3 inhibitors, K-Ras inhibitors, PI3 kinase inhibitors, JAK inhibitors, STAT inhibitors, Raf kinase inhibitors, Akt inhibitors, mTOR inhibitor, P70S6 kinase inhibitors, inhibitors of the WNT pathway and so called multi-targeted kinase inhibitors. Preferred signal transduction inhibitors include gefitinib (Iressa™), cetuximab (Erbitux™), erlotinib (Tarceva™), trastuzumab (Herceptin™), sunitinib (Sutent™), and imatinib (Gleevec™).

Additional examples of signal transduction inhibitors which may be used in conjunction with a compound of the invention and pharmaceutical compositions described herein include BMS 214662 (Bristol-Myers Squibb), lonafarnib (Sarasar™), pelitrexol (AG 2037), matuzumab (EMO 7200), nimotuzumab (TheraCIM h-R3™), panitumumab (Vectibix™), Vandetanib (Zactima™), pazopanib (SB 786034), ALT 110 (Alteris Therapeutics), BIBW 2992 (Boehringer Ingelheim), and Cervene™ (TP 38). Other examples of signal transduction inhibitor include PF-2341 066 (Pfizer), PF-299804 (Pfizer), canertinib, pertuzumab (Omnitarg™), Lapatinib (Tycerb™), pelitinib (EKB 569), miltefosine (Miltefosin™), BMS 599626 (Bristol-Myers Squibb), Lapuleucel-T (Neuvenge™), NeuVax™ (E75 cancer vaccine), Osidem™, mubritinib (TAK-165), panitumumab (Vectibix™), lapatinib (Tycerb™), pelitinib (EKB 569), and pertuzumab (Omnitarg™). Other examples of signal transduction inhibitors include ARRY 142886 (Array Biopharm), everolimus (Certican™), zotarolimus (Endeavor™), temsirolimus (Torisel™), and AP 23573 (ARIAO). Additionally, other signal transduction inhibitors include XL 647 (Exelixis), sorafenib (Nexavar™), LE-AON (Georgetown University), and GI-4000 (Globelmmune). Other signal transduction inhibitors include ABT 751 (Abbott), alvocidib (flavopiridol), BMS 387032 (Bristol Myers), EM 1421 (Erimos), indisulam (E 7070), seliciclib (CYC 200), BIO 112 (Onc Bio), BMS 387032 (Bristol-Myers Squibb), PO 0332991 (Pfizer), and AG 024322 (Pfizer).

Among the signal transduction inhibitors that agents of the invention will be useful in in combination, other erbB family inhibitors, exemplified by erlotinib, gefitinib, lapatinib, icotinib, afatinib, neratinib, peletinib and dacomitinib, are recognized to be of especial interest. All of these compounds have enough wild-type erbB kinase inhibitory activity to have mechanism-based dose limiting toxicities, but all can be dosed at tolerable levels, and demonstrate good clinical activity. One of their main weaknesses is that tumors which respond well to these medications tend to have erbB mutations which make the tumor unusually susceptible to the inhibitor, but which when combined with a second mutation, tend to make the tumor very resistant to these agents. The selection pressures which accelerate this process have been discussed above. Compounds of the current invention target the main resistance mutants, and because they have very little activity against the wild type enzymes will not add appreciably to mechanism based toxicity. However, they will put the evolving double mutants under the same selection disadvantage as the original susceptible mutants, and will therefore greatly slow or perhaps prevent altogether the emergence of the resistant strains. Therefore, this combination will prove to be clinically very useful.

This invention contemplates the use of compounds of the invention together with classical antineoplastic agents. Classical antineoplastic agents include hormonal modulators such as hormonal, anti-hormonal, androgen agonist, androgen antagonist and anti-estrogen therapeutic agents, histone deacetylase (HOAC) inhibitors, gene silencing agents or gene activating agents, ribonucleases, proteosomics, Topoisomerase I inhibitors, Camptothecin derivatives, Topoisomerase II inhibitors, alkylating agents, anti-metabolites, poly(AOP-ribose) polymerase-1 (PARP-1) inhibitor, microtubulin inhibitors, antibiotics, plant derived spindle inhibitors, platinum-coordinated compounds, gene therapeutic agents, antisense oligonucleotides, vascular targeting agents (VTAs), and statins.

Examples of antineoplastic agents used in combination with compounds of the invention include Velcade (bortezomib), 9-aminocamptothecin, belotecan, camptothecin, diflomotecan, edotecarin, exatecan (Daiichi), gimatecan, 10-hydroxycamptothecin, irinotecan HCl (Camptosar), lurtotecan, Orathecin (rubitecan, Supergen), topotecan, camptothecin, 10-hydroxycamptothecin, 9-aminocamptothecin, irinotecan, edotecarin, topotecan, aclarubicin, adriamycin, amonafide, amrubicin, annamycin, daunorubicin, doxorubicin, elsamitrucin, epirubicin, etoposide, idarubicin, galarubicin, hydroxycarbamide, nemorubicin, novantrone (mitoxantrone), pirarubicin, pixantrone, procarbazine, rebeccamycin, sobuzoxane, tafluposide, valrubicin, Zinecard (dexrazoxane), nitrogen mustard N-oxide, cyclophosphamide, altretamine, AP-5280, apaziquone, brostallicin, bendamustine, busulfan, carboquone, carmustine, chlorambucil, dacarbazine, estramustine, fotemustine, glufosfamide, ifosfamide, lomustine, mafosfamide, mechlorethamine, melphalan, mitobronitol, mitolactol, mitomycin C, mitoxatrone, nimustine, ranimustine, temozolomide, thiotepa, and platinum coordinated alkylating compounds such as cisplatin, Paraplatin (carboplatin), eptaplatin, lobaplatin, nedaplatin, Eloxatin (oxaliplatin, Sanofi), streptozocin, satraplatin, and combinations thereof.

The invention also contemplates the use of the compounds of the invention together with dihydrofolate reductase inhibitors (such as methotrexate and trimetrexate glucuronate), purine antagonists (such as 6-mercaptopurine riboside, mercaptopurine, 6-thioguanine, cladribine, clofarabine (Clolar), fludarabine, nelarabine, and raltitrexed), pyrimidine antagonists (such as 5-fluorouracil), Alimta (premetrexed disodium), capecitabine (Xeloda™), cytosine arabinoside, Gemzar™ (gemcitabine), Tegafur, doxifluridine, carmofur, cytarabine (including ocfosfate, phosphate stearate, sustained release and Iiposomal forms), enocitabine, 5-azacitidine (Vidaza), decitabine, and ethynylcytidine) and other antimetabolites such as eflornithine, hydroxyurea, leucovorin, nolatrexed (Thymitaq), triapine, trimetrexate, and N-(5-[N-(3,4-dihydro-2-methyl-4-oxoquinazolin-6-ylmethyl)-N-methylamino]-2thenoyl)-L-glutamic acid, and combinations thereof.

Other examples of classical antineoplastic cytotoxic agents used in combination therapy with a compound of the invention, optionally with one or more other agents include Abraxane (Abraxis BioScience, Inc.), Batabulin (Amgen), Vinflunine (Bristol-Myers Squibb Company), actinomycin D, bleomycin, mitomycin C, neocarzinostatin (Zinostatin), vinblastine, vincristine, vindesine, vinorelbine (Navelbine), docetaxel (Taxotere), Ortataxel, paclitaxel (including Taxoprexin a DHA/paclitaxel conjugate), cisplatin, carboplatin, Nedaplatin, oxaliplatin (Eloxatin), Satraplatin, Camptosar, capecitabine (Xeloda), oxaliplatin (Eloxatin), Taxotere alitretinoin, Canfosfamide (Telcyta™), DMXAA (Antisoma), ibandronic acid, L-asparaginase, pegaspargase (Oncaspar™), Efaproxiral (Efaproxyn™—radiation therapy)), bexarotene (Targretin™), Tesmilifene, Theratope™ (Biomira), Tretinoin (Vesanoid™), tirapazamine (Trizaone™), motexafin gadolinium (Xcytrin™) Cotara™ (mAb), and NBI-3001 (ProtoxbTherapeutics), polyglutamate-paclitaxel (Xyotax™) and combinations thereof.

### EXAMPLES

### Experimentals

Synthesis of Compounds of the Invention

The compounds of the current invention can be made by a variety of processes, which are known to one of skill in the art, and some synthetic schemes to make these compounds are illustrated below.

The compounds of the current invention can be regarded as consisting of four concatenated components; the A-ring (A′) which may be monocyclic or bicyclic, the central azine ring, (B′) which is usually a 2,4,(5)-substituted pyrimidine, (or a bicyclic homologue), the aniline (C′) or 3-aminopyridine moiety, and the electrophilic side chain (D′) on that C′ ring to form the concatenated A′-B′-C′-D′ structure. This allows for each of the four components to be used combinatorially with the other three components, allowing for a large number of analogues to be synthesized from relatively few building blocks in a parsimonious and efficient fashion.

Several syntheses illustrated in this document start by preparing the A′ subunit, and then attaching it to the B′ subunit, to form an A′-B′ moiety, via a variety of chemistries known to one of skill in the art, many of which are exemplified below. The C-unit is now attached, by displacement of a halogen atom on the B′-ring by the C-unit free primary amine, to form an A′-B′-C′ entity. The C-entity has an incipient primary amine unmasked, either by reduction of a precursor group such as nitro or azido, or by deprotection of a protected primary amine, and then the D-subunit is attached to this free amine via an acylation or sulfonation reaction.

In many cases, the D-subunit, although acting as an electrophile in vivo is in fact a rather weak electrophile and can survive a reasonable variety of chemical reaction conditions, which appears to be especially true of acrylamido and crotonamido D′ species. Furthermore, the A′-subunits once incorporated into larger entities can be of quite different chemical reactivities to one another, sometimes allowing them to be modified late in the synthesis, and other times leaving them generally inert during subsequent reactions. The azine ring in an A′-B′-C′ concatenated entity tends to be chemically of low activity. This allows for other reaction orders to be used. For example, if the A′ moiety is somewhat chemically reactive sometimes a final chemical modification can be made to the A′-moiety, after the A′-B′ coupling, or after the A′-B′-C′ entity is assembled, or sometimes even after complete assembly of an A′-B′-C′-D′ entity. Or an A′-B′-C′ entity can be assembled, and then the C-ring can be modified, for example by displacement of an electrophilic fluorine ortho to a nitro group by an R³ amine, thiol, or alcoholate nucleophile. One of skill in the art can find many opportunities for such deviations from the “canonical” linear A to D assembly, and several such reaction sequences are illustrated in the reactions below. See also, PCT/US2017/012466.

A′-B′ Couplings

The central azine rings of the invention can all be commercially obtained with two halogen atoms (Q¹ and Q²) in a 1,3-relationship to one another, and one of these halogens can always be displaced preferentially to the other (even if the original azine was symmetric). Normally the more reactive Q group, which in the case of 2,4-dichloropyrimidines, is the 4-chloro, can be displaced by a nitrogen or carbon nucleophile in good yields, leaving the other group to be displaced later by an amine nucleophile under potentially drastic conditions. In the case of pyrimidines the A′-B′ biaryl moiety is normally a 4-substituted-2-chloropyrimidine, and most syntheses disclosed in this patent use such intermediates. They are listed as the A intermediates in the experimental section.

The biaryls described here may be linked together via either a C or N atom on A′ to a C atom of the central B′ azine. If the A′ moiety is linked through a 6-membered ring then the biaryl has to be prepared by a carbon-carbon bond formation. Such syntheses are very well known to one of skill in the art, and can involve, Stille, Negishi Ullmann or Suzuki type catalyzed reactions, or many variants thereof, along with numerous other reaction sequences, all known to one of skill in the art. If the linking portion of the A′-moiety is a five membered aromatic ring containing an N atom, the ring can often be attached through either a C atom or an N atom. Proton extraction can drive one towards C or N alkylation depending on the exact system and the nature of the counterions and catalysts present, and especially with indole-like aromatics, N versus C alkylation is usually well controllable by one of skill in the art.

Furthermore, once one has formed the A′-B′ biaryl, it is often possible to do reactions selectively on the A′-portion of the molecule, as the second halogen is often of quite low reactivity. Several such examples are illustrated in the experimental disclosures below. See also, PCT/US2017/012466.

The C′-subunit contains a primary amine which will be used to displace the second Q species. This can be done under conditions of acid catalysis (most common method) or basic catalysis, or with transition metal catalysis, and all of these are well exemplified in the prior art, eg. Buchwald reactions, and in some of the examples below. Although it is not specifically discussed above, or exemplified below, one can also have a halogen replace the primary amine of the aniline, and displace the second Q group with ammonia or suitable precursor (azide, trifluoroacetamide, sulfonamide, etc., modify it as required, and then displace the halogen on the C-unit under conditions of transition metal catalysis, followed by removal of the activating group from nitrogen, if such were used. The C′-moiety also contains a precursor for the amine used to attach the electrophilic D′-moiety, especially nitro, or as a protected amine, especially t-Bocamino. The advantage of a 3-nitro is that it can activate a leaving group ortho to it at the 4-position to nucleophilic substitution, allowing the easy introduction of many R³ side chains especially amines at that position. Having the 4-substituent on the C′-moiety fluorine is especially advantageous for facilitation of this reaction, but other side chains, including carbon linked ones can be made by having other halogens at the 4-position, and then doing transition metal coupled reactions, such as Stille, Suzuki, Sonogashira and Buchwald reactions.

A′-B′-C′ entities can be readily constructed to facilitate modification on either the A′ or the C′ moieties. As the amine on the C′ aromatic ring to be linked to the D′-electrophile is a primary amine, it needs to be protected during the B′-C′ coupling, so there is almost invariably a need for a reaction on this position, and most syntheses revealed herein have such a reaction. However, if the 3-amine precursor is highly activating, to displacement of a 4-halogen (eg nitro) one can do the (A′-)B′-C′ coupling prior to introducing R³, and some examples of the introduction of R³ onto an A′-B′-C′ entity are disclosed below. See also, PCT/US2017/012466.

Usually the electrophilic D′ moiety is added at the end of the synthesis to give the completed compound of the invention. However, as mentioned above, several of the D′-groups are of low enough chemical reactivity, especially when present as relatively weakly electron-withdrawing amides, to allows for a variety of transformations to be done on completed A′-B-′C′-D′ entities, especially when introducing certain groups onto the A′-moiety, which might have interfered with some of the earlier chemistry, and some such examples are also disclosed below. See also, PCT/US2017/012466.

In principle, the B′-C′ coupling should work with the complete C′-D′ fragment preformed, as the aniline/3-aminopyridine fragment with the D′ unit attached is going to have at least as nucleophilic a primary amine for the B′-C′ coupling as most of the “monomeric” C′ moieties one would use. Here, the same C′ moiety starting materials can be employed as previously, but one needs to protect the 1-amine, unmask the 3-amine, acylate or sulfonate it, and then deprotect the 1-amine. Then one can use this C′-D′ fragment to couple to a suitable A′-B′ fragment to form the final A′-B′-C′-D′ entity, and several such syntheses are disclosed below. See also, PCT/US2017/012466.

Thus, the reactions described within this patent application enable one to prepare not only the exemplified compounds of the invention, but using the reactions described herein, and variants of them in the chemical literature ready available to one of skill in the art, also allows one to produce many other compounds including those claimed within this patent application, which are not specifically exemplified. Furthermore, as mentioned earlier, because of the modular nature of the compounds of the invention, and the ability to make several examples of each module, one has the ability to produce a very large number of compounds using the chemistry enabled by disclosures in this application. See also, PCT/US2017/012466. For example, one can use 3-aminopyridyl C′ moieties in place of the 3-anilino C′ moieties in combination with most of the A′-B′ moieties disclosed in this patent using reaction conditions discussed in this application. As another example, the displacement of the 4-fluoro group on the nitroanilines of the precursor to the C′-moiety can be displaced by a very wide array of amines, using the conditions disclosed in this document. See also, PCT/US2017/012466.

Scheme 1 shows a generic scheme to make compounds of the current invention, illustrated with A being A¹, a 6,5-bicyclic system connected to the central azine ring through the 1-(3-)position of the five membered ring, and Y being an α,β-unsaturated enamide. The synthesis involves preparing five components, a suitably substituted azine 1A, which contains leaving groups Q¹ and Q², usually but not necessarily halogens, ortho and para to the obligate nitrogen, a suitable A group 2A (A¹ in this case), where T¹ represents a group which is a suitable coupling partner for Q¹, an appropriate meta-nitroaniline, or 3-amino-5-nitropyridine 4A with a leaving group Q³, probably halogen, an appropriate side chain R³T², 5A, where T², usually hydrogen, is an appropriate leaving group for coupling via displacement of Q³, and lastly an appropriate electrophile 9A, here illustrated by an enoyl chloride, where Q⁴ is an appropriate leaving group for coupling with an aromatic amine. Some of the components A, 2A, 4A, 5A and 9A, may be commercially available, and if they are not, they can be made by methods known to one of ordinary skill in the art.

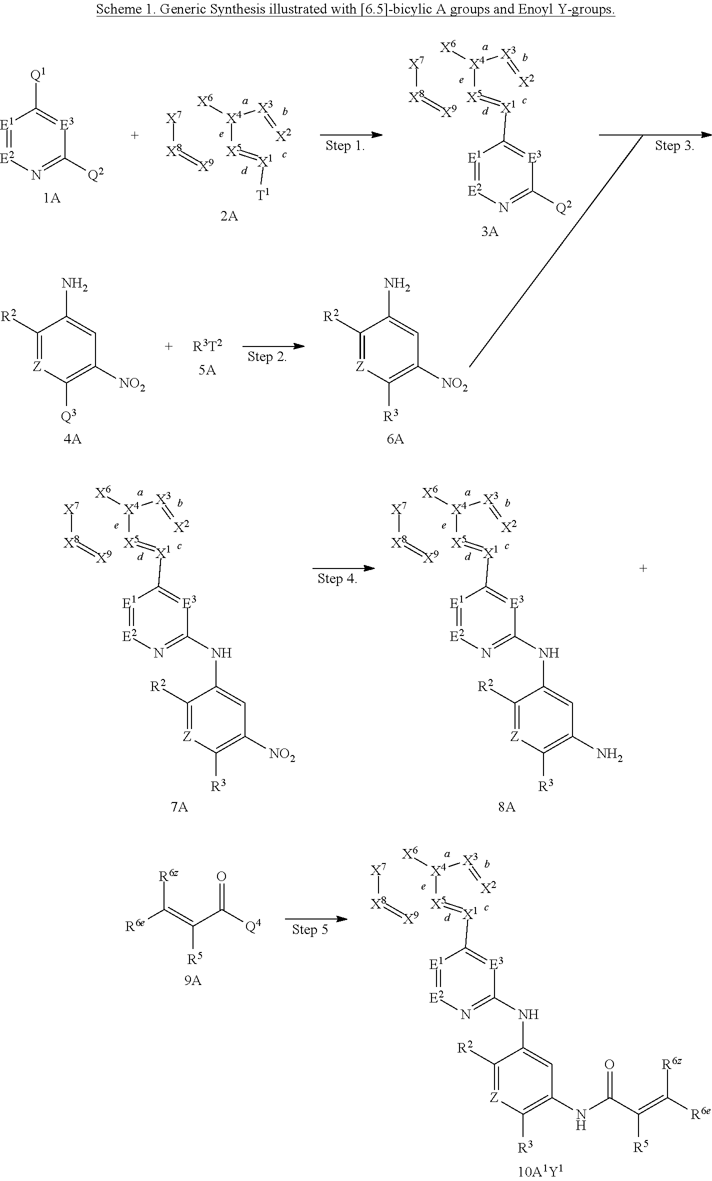

This synthetic scheme describes a commonly used strategy, which has essentially A′ linked sequentially to B′, which is then attached to a C′ moiety, which already has the R³ side chain attached, and then after reduction of the nitro group, D′ is attached to complete the synthesis. In the first step of Scheme 1, the azine 1A, being the B′ moiety is coupled at its 4-position with the A′ moiety a [6.5]-bicycle, 2A, either at its 1- or 3-position, with an overall loss of Q¹T¹ to form intermediate 3A. Such couplings will frequently be a displacement of halide ion by nitrogen, or a nitrogen based anion, but equally can involve formation of a carbon-carbon bond, by methods familiar to one of skill in the art, such as Stille, Negishi or Suzuki couplings, or Freidel-Crafts aryl substitutions. In Step 2 Q³ on 4A is displaced by 5A, with a loss of Q³T2, to form intermediate 6A, the complete C′ moiety. Such couplings will frequently be a displacement of halide ion by nitrogen, or a nitrogen based anion, but equally can involve formation of a carbon-carbon bond, by methods familiar to one of skill in the art, such as Stille, Negishi or Suzuki couplings.

In step 3, the amino nitrogen of 6A is used to displace Q² from the A′-B′ moiety, intermediate 3A, to form an A′-B′-C′ concatenated intermediate 7A, using methods known to one of skill in the art. The nitro group of 7A is then reduced to the amino group of intermediate 8A, using methods such as iron/acetic acid or catalytic hydrogenation, well known to those of ordinary skill in the art. The synthesis of the complete A′-B′-C′-D′ final product, 10A¹Y¹ in this illustrative general case, is completed by an amide coupling of amine 8A with a suitable enoic acid derivative 9A, where the leaving group Q⁴ can be a halide, activated ester, acid plus coupling agent, or other activated acid derivative suitable for peptide coupling, known to one of skill in the art. Other compounds of the invention are made by analogous processes, with different A and Y groups, using appropriate starting materials and coupling reactions, all of which are well known to one of skill in the art.

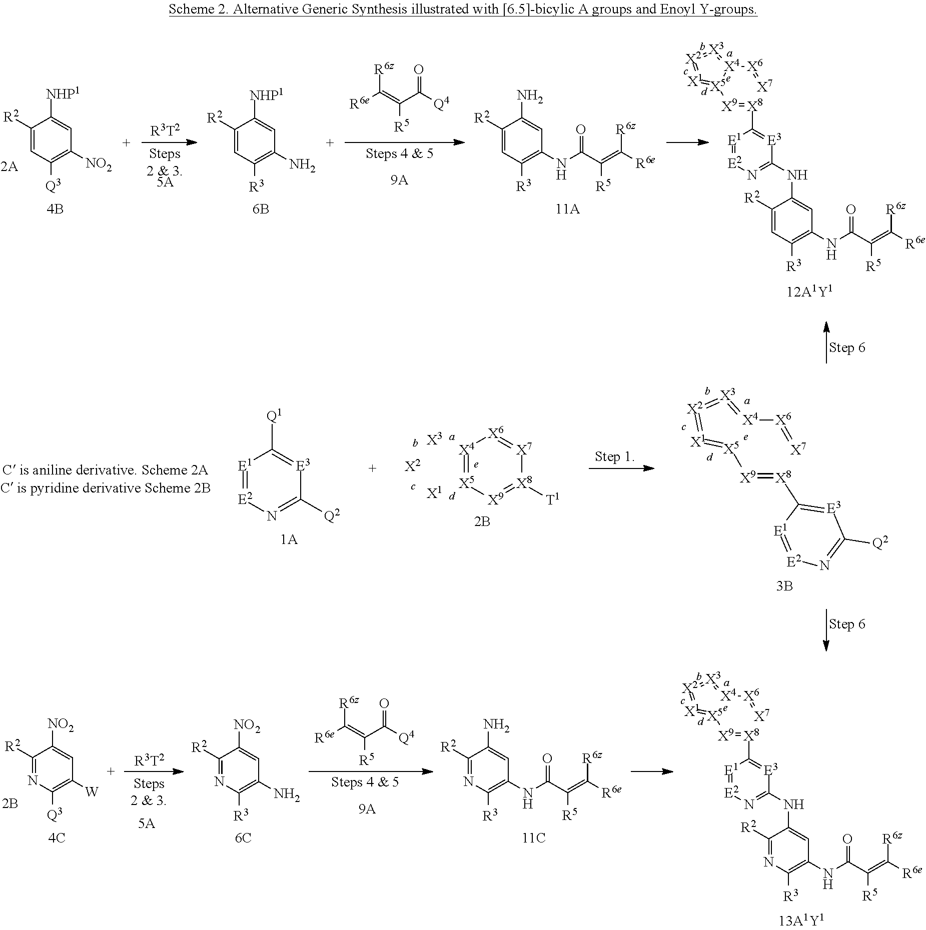

In Scheme 2, one of the alternative strategies is illustrated, using similar components as in Scheme 1. In this case the illustrative A¹ (A′) component is a 5-linked 6,5-azaaromatic system. The first step is the same as before with the A′ moiety 2B being coupled to the B′ azine 1A by the same types of reactions described previously to form 3B the A′-B′ entity, as in Scheme 1. Two alternative routes 2A and 2B are illustrated here to form the C′-D′ fragment, which will be coupled late, or at the end of the sequence with the A′-B′, as optimal chemistries have to differ rather more than needed in Scheme 1 for anilines and pyridines.

In scheme 2A, the starting nitroaniline 4B is similar to 4A, except that the amine is suitably protected. The R³ moiety is introduced as before by displacement of Q³, and then the nitro group is reduced to an unprotected amine to give the appropriate C′ fragment 6B. This is then acylated with the D′ moiety 9A on the free amine, and the coupling ready C′D′ entity 11B is completed by removal of the protecting group from the original amine. In scheme 2B the high, and selective reactivity of halonitropyridines allows for an easy preparation of 4C moieties, where W is a group that can be readily turned into an amine later. This can even be a proton, as after R³ has been introduced by 5A displacement of Q³, the 5-position of the pyridine is quite highly activated to electrophilic aromatic substitution. Replacement of W with a free amine under non-reducing conditions will give the C′ entity as a nitroaniline 6C, with the free amine at the correct position for acylation by the D′ entity 9A. A mild reduction of the 3-nitro group to the amine completes the preparation of this C′-D′ moiety 11C, in a form ready for the final coupling.

In what in many cases will be the last step of the synthesis, the Q2 fragment on 3A is displaced by the free amine on 11B or 11C, using the same sorts of couplings that were used to couple the A‘B’ fragment to the C′ moiety as described for Scheme 1, to produce entities 12A¹Y¹ and 13A¹Y¹. Many of the same conditions can be employed here, as acrylamide D′ moieties especially are often robust enough to survive the amine displacement reactions used here. Alternatively, one can use precursors to the final D′ electrophile in this reaction and activate the final electrophilic species, after this coupling is complete.

For examples of synthesis, see international application no. PCT/US2017/012466, the disclosure of which is hereby incorporated in its entirety for all intended purposes.

### INTERMEDIATES

### A1. 2-Chloro-4-(3-(N,N-dimethylamino)-6-methyl-pyrazolo[4,3-c]pyridin-1-yl) pyrimidine

### Methyl 4-hydroxy-6-methylnicotinate

A solution of 4-hydroxy-6-methylnicotinic acid (50.0 g, 261.4 mmol, 1.0 eq) in methanol (750 mL) was cooled to 0° C. and treated drop-wise over 20 minutes with SOCl₂ (205.0 g, 1533 mmol, 5.0 eq). The reaction mixture was allowed to warm to 25° C., heated at 70° C. for 20 hours, and concentrated. The resultant residue was washed with methanol (50 mL), filtered and dried to provide product methyl 4-hydroxy-6-methylnicotinate (54.0 g, 99%). ¹H NMR (300 MHz, CDCl₃): δ 9.05 (s, 1H), 7.13 (s, 1H), 4.09 (s, 3H), 2.90 (s, 3H). ESI-MS (m/z): 168.0 (M+H)⁺.

### 4-Hydroxy-6-methylnicotinamide

A suspension of product methyl 4-hydroxy-6-methylnicotinate (25.0 g, 149 mmol, 1.0 eq) in aqueous ammonia (500 mL) was heated at 50° C. for 20 hours. The reaction mass was then concentrated, and the resultant residue was washed with a mixture of diethyl ether and DCM (100 mL, 8:2). The resultant solids were collected and dried under reduced pressure to provide product 4-hydroxy-6-methylnicotinamide as an off-white solid (20.0 g, 88%). ESI-MS (m/z): 151.0 (M−H)⁻.

### 4-Chloro-6-methylnicotinonitrile

A suspension of product 4-hydroxy-6-methylnicotinamide (20.0 g, 131.5 mmol, 1.0 eq) in POCl₃ (62 mL, 580 mmol, 4.4 eq) was heated at 110° C. for 5 hours. The mixture was allowed to cool to 10° C., and quenched with aqueous Na₂CO₃ (200 ml). The mixture was then extracted with EtOAc (250 mL×3), and the combined organic layers were washed with brine, dried over anhydrous sodium sulfate, and concentrated. The resultant residue was purified by chromatography (silica gel; 4-5% EtOAc in petroleum ether as eluting solvent) to provide product 4-chloro-6-methylnicotinonitrile as an off-white puffy solid (8.0 g, 40%). ¹H NMR (300 MHz, DMSO-d₆): δ 8.96 (s, 1H), 7.79 (s, 1H), 2.50 (s, 3H). ESI-MS (m/z): 153.0 (M+H)⁺.

### 3-Amino-6-methyl-1H-pyrazolo[4,3-c]pyridine

To a mixture of compound 4-chloro-6-methylnicotinonitrile (8.0 g, 52.6 mmol, 1.0 eq) in n-butanol (80 mL) was added hydrazine hydrate (7.9 g, 158 mmol, 3.0 eq), the mixture was heated to reflux under nitrogen for 12 h. The reaction mixture was allowed to cool to room temperature, the solid was collected by filtration and washed with ethyl acetate (30 mL×2). Dried to give compound 3-amino-6-methyl-1H-pyrazolo[4,3-c]pyridine (5.0 g, 64%). ¹H NMR (300 MHz, DMSO-d₆) δ 11.61 (br, 1H), 8.80 (s, 1H), 6.97 (s, 1H), 5.69 (s, 2H), 2.46 (s, 3H). ESI-MS (m/z): 149.0 (M+H)⁺.

### 2-Chloro-4-(3-amino-6-methyl-pyrazolo[4,3-c]pyridin-1-yl)pyrimidine

t-BuOK (4.16 g, 37.1 mmol, 1.1 eq) was added carefully to a solution of 3-amino-6-methyl-1H-pyrazolo[4,3-c]pyridine (5.0 g, 33.7 mmol, 1.0 eq) in DMF (50 mL) at 0° C. The mixture was stirred at this temperature for 10 minutes. Then a solution of 2,4-dichloropyrimidine (5.53 g, 37.1 mmol, 1.1 eq) in DMF (25 mL) was added drop wise. The mixture was stirred at RT for 2 h. After completion, the mixture was diluted with water (250 mL), filtered, washed with water (20 mL×2). The filter cake was then dried and purified by column chromatography on silica to give the desired product 2-chloro-4-(3-amino-6-methyl-pyrazolo[4,3-c]pyridin-1-yl)pyrimidine (2.5 g, 28%). ¹H NMR (300 MHz, DMSO-d₆): δ 9.04 (s, 1H), 8.59 (d, J=5.7 Hz, 1H), 8.13 (s, 1H), 7.53 (d, J=5.7 Hz, 1H), 2.63 (s, 3H). ESI-MS (m/z): 260.9 (M+H)⁺.

### 2-Chloro-4-(3-(N,N-dimethylamino)-6-methyl-pyrazolo[4,3-c]pyridin-1-yl)pyrimidine

NaH (0.84 g, 21.1 mmol, 2.2 eq) was added carefully to a solution of 2-chloro-4-(3-amino-6-methyl-pyrazolo[4,3-c]pyridin-1-yl)pyrimidine (2.5 g, 9.6 mmol, 1.0 eq) in DMF (25 mL) at 0° C., and the mixture was stirred at this temperature for 30 minutes. Then MeI (2.98 g, 21.1 mmol, 2.2 eq) was added dropwise. After addition, the mixture was warmed to RT and stirred for 2 hours till completion. The mixture was poured into water (100 mL), and extracted with EA (50 mL×3). The combined organic layers were washed with brine twice, dried over sodium sulphate, concentrated and purified by silica column chromatography to give the desired product 2-chloro-(3-(N,N-dimethylamino)-6-methyl-pyrazolo[4,3-c]pyridin-1-yl) pyrimidine (0.2 g, 7%). ¹H NMR (300 MHz, DMSO-d₆): δ 9.26 (s, 1H), 8.70 (d, J=5.7 Hz, 1H), 8.34 (s, 1H), 7.77 (d, J=5.7 Hz, 1H), 3.32 (s, 6H), 2.70 (s, 3H). ESI-MS (m/z): 288.9 (M+H)⁺.

### A2. 2-Chloro-4-(7-cyano-1,3-dimethyl-1H-indol-5-yl)pyrimidine

### 5-Bromo-7-cyano-1,3-dimethyl-1H-indole

To a stirred solution of 3-methyl-5-bromo-7-cyanindole (1.17 g, 5 mmol) in THF (20 mL) at 0° C. was added NaH (260 mg, 6.5 mmol) portion wise. The mixture was stirred at 0° C. for 30 min, then MeI (781 mg, 5.5 mmol) was added drop wise. After the addition, the reaction mixture was stirred at room temperature for 2 h and quenched with water, the solution was extracted with EtOAc (20 mL×3) and dried over sodium sulfate, filtered and concentrated in vacuo to give a crude residue, which was purified on silica gel chromatography to give 5-bromo-7-cyano-1,3-dimethyl-1H-indole (694 mg, 56%). ¹H NMR (300 MHz, CDCl₃): δ 7.87 (d, J=1.5 Hz, 1H), 7.60 (d, J=1.5 Hz, 1H), 6.88 (s, 1H), 4.06 (s, 3H), 2.28 (s, 3H).

### 7-Cyano-1,3-dimethyl-5-(4,4,5,5-tetramethyl-1,3,2-dioxaborolan-2-yl)-1H-indole

To a solution of 5-bromo-7-cyano-1,3-dimethyl-1H-indole (523 mg, 2.1 mmol, 1.0 eq) in dioxane (5 mL) was added Bis(pinacolato)diboron (592 mg, 2.3 mmol, 1.1 eq), KOAc (125 mg, 6.3 mmol, 3.0 eq) and Pd(dppf-)Cl₂ (124 mg, 0.168 mmol, 0.08 eq). The mixture was purged with nitrogen three times, then heated at 85° C. under nitrogen for 6 hours. After TLC and LCMS indicated completion, the mixture was filtered, the filtrate was concentrated and purified by silica column affording 7-cyano-1,3-dimethyl-5-(4,4,5,5-tetramethyl-1,3,2-dioxaborolan-2-yl)-1H-indole (350 mg, 56%). ¹H NMR (300 MHz, CDCl₃): δ 8.23 (s, 1H), 7.99 (s, 1H), 6.85 (s, 1H), 4.09 (s, 3H), 2.33 (s, 3H), 1.39 (s, 12H).

### 2-Chloro-4-(7-cyano-1,3-dimethyl-1H-indol-5-yl)pyrimidine

To a solution of 7-cyano-1,3-dimethyl-5-(4,4,5,5-tetramethyl-1,3,2-dioxaborolan-2-yl)-1H-indole (296 mg, 1.0 mmol, 1.0 eq) in dioxane (5 mL) and water (1 mL) was added 2,4-dichloropyrimidine (162 mg, 1.1 mmol, 1.1 eq), Pd(PPh₃)₄ (115 mg, 0.1 mmol, 0.1 eq) and K₂C03 (411 mg, 3.0 mmol, 3.0 eq). The mixture was purged with nitrogen three times, then heated at 100° C. under nitrogen for 3 hours. After TLC and LCMS indicated completion, the mixture was filtered, the filtrate was concentrated and purified by silica column to give 2-chloro-4-(7-cyano-1,3-dimethyl-1H-indol-5-yl)pyrimidine (164 mg, 58%). ¹H NMR (300 MHz, DMSO-d₆): δ 8.65 (d, J=5.1 Hz, 1H), 8.55 (s, 1H), 8.29 (s, 1H), 7.70 (d, J=5.1 Hz, 1H), 6.95 (s, 1H), 4.13 (s, 3H), 2.35 (s, 3H).

### A3. 2-Chloro-4-(1,N-(tert-Butoxycarbonyl)-7-cyano-3-methyl-pyrrolo[2,3-c]pyridin-4-yl) pyrimidine

### 3-Amino-2-cyanopyridine

To a solution of 2-cyano-3-fluoropyridine (5.0 g, 41 mmol, 1.0 eq) in DMSO (50 mL) at 70° C. was bubbled ammonia gas for 6 h. After completion and cooling to room temperature, water (80 mL) was added, extracted with EA (100 mL×3), the combined organic layers were washed with brine, dried over Na₂SO₄, filtered and concentrated to give a crude residue, which was purified by silica column to give 3-amino-2-cyanopyridine (3.0 g, 61%)._H NMR (300 MHz, DMSO-d₆): δ 7.85 (d, J=3.0 Hz, 1H), 7.33-7.30 (m, 1H), 7.21-7.19 (m, 1H), 6.30 (br, 2H).

### 3-Amino-4,6-dibromo-2-cyanopyridine

3-Amino-2-cyanopyridine (3.0 g, 25.2 mmol, 1.0 eq) was dissolved in DMF (30 mL) and N-bromosuccinimide (10.1 g, 56.7 mmol, 2.3 eq) was added. The solution was stirred for 20 h at room temperature before water (40 mL) was added. The mixture was extracted with EA (50 mL×3), the combined organic layer was washed with water and brine, dried over magnesium sulfate and concentrated in vacuum. The residue was purified by column chromatography to afford 3-Amino-4,6-dibromo-2-cyanopyridine (4.0 g, 57%). ¹H NMR (300 MHz, CDCl₃): δ 7.74 (s, 1H), 4.95 (br, 2H). ESI-MS (m/z): 275.6 (M−H)⁻.

### 3-(Allylamino)-4,6-dibromo-2-cyano-pyridine

To a solution of 3-amino-4,6-dibromo-2-cyanopyridine (4.0 g, 14.4 mmol, 1.0 eq) in THF (40 mL) was added a solution of t-BuOK (2.4 g, 21.6 mmol, 1.5 eq) in THF drop wise at 0° C. The mixture was kept at this temperature for 10 min. Then 3-bromoprop-1-ene (1.7 g, 14.4 mmol, 1.0 eq) was added dropwise and the mixture was stirred at RT for 2 h. After TLC and LCMS indicated completion, the mixture was quenched with water (50 mL), extracted with EA (50 mL×3). The combined organic layers were washed with brine (100 mL), dried over sodium sulfate, concentrated affording a crude residue, which was purified by column chromatography to afford 3-(allylamino)-4,6-dibromo-2-cyano-pyridine (1.2 g, 26%). ¹H NMR (300 MHz, CDCl₃): δ 7.72 (s, 1H), 6.02-5.86 (m, 1H), 5.36-5.25 (m, 1H), 4.88-4.86 (m, 1H), 4.41-4.31 (m, 2H), 4.04 (br, 1H).

### 5-Bromo-7-cyano-3-methyl-1H-pyrrolo[2,3-c]pyridine

To a solution of 3-(allylamino)-4,6-dibromo-2-cyano-pyridine (8.0 g, 25.2 mmol, 1.0 eq) in MeCN (80 mL) was added TEA (7.64 g, 75.7 mmol, 3.0 eq), Pd(OAc)₂ (610 mg, 2.52 mmol, 0.1 eq) and tri-o-tolylphosphine (1.53 g, 5.04 mmol, 0.2 eq). The mixture was purged with nitrogen three times, then refluxed for 2 hours. After TLC and LCMS indicated completion, the mixture was diluted with water (150 mL), MeCN was removed under reduced pressure, extracted with EA (100 mL×3). The combined organic layers were washed with sat. NH⁴Cl (100 mL), dried over sodium sulfate, concentrated and purified on silica column affording 5-bromo-7-cyano-3-methyl-1H-pyrrolo[2,3-c]pyridine (2.5 g, 42%). ESI-MS (m/z): 235.8 (M+H)⁺.

### 1,N-(tert-Butoxycarbonyl)-7-cyano-3-methyl-1H-pyrrolo[2,3-c]pyridine

To a solution of 5-bromo-7-cyano-3-methyl-1H-pyrrolo[2,3-c]pyridine (2.5 g, 10.6 mmol, 1.0 eq) in DCM (30 mL) was added DMAP (130 mg, 1.06 mmol, 0.1 eq) and TEA (2.1 g, 21.2 mmol, 2.0 eq). A solution of Boc₂O (2.77 g, 12.7 mmol, 1.2 eq) in DCM (10 mL) was added dropwise. After addition, the reaction mixture was stirred at room temperature overnight. The reaction mixture was concentrated in vacuo to give a crude residue, which was purified on silica gel chromatography to give 1,N-(tert-butoxycarbonyl)-7-cyano-3-methyl-1H-pyrrolo[2,3-c]pyridine (1.5 g, 42%). ¹H NMR (300 MHz, CDCl₃): δ 7.80 (s, 1H), 7.59 (s, 1H), 2.25 (s, 3H), 1.70 (s, 9H). ESI-MS (m/z): 279.8 (M+H−56)⁺.

### (1,N-(tert-butoxycarbonyl)-7-cyano-3-methyl-H-pyrrolo[2,3-c]pyridine-5-yl)boronic Acid

To a solution of 1,N-(tert-butoxycarbonyl)-7-cyano-3-methyl-1H-pyrrolo[2,3-c]pyridine (672 mg, 2.0 mmol, 1.0 eq) in dioxane (6 mL) was added bis(pinacolato)diboron (609.6 mg, 2.4 mmol, 1.2 eq), KOAc (588 mg, 6.0 mmol, 3.0 eq) and Pd(dppf)Cl₂ DCM (73 mg, 0.1 mmol, 0.05 eq). The mixture was purged with nitrogen three times, then heated at 85° C. under nitrogen for 6 hours. After TLC and LCMS indicated completion, the mixture was filtered, the filtrate was concentrated and purified on silica column affording (1,N-(tert-butoxycarbonyl)-7-cyano-3-methyl-1H-pyrrolo[2,3-c]pyridin-5-yl)boronic acid (200 mg, 33%). ¹H NMR (300 MHz, DMSO-d₆): δ 8.18 (s, 1H), 7.82 (s, 1H), 7.51 (br, 2H), 2.28 (s, 3H), 1.63 (s, 9H). ESI-MS (m/z): 245.9 (M+H−56)⁺.

### 2-Chloro-4-(1,N-(tert-Butoxycarbonyl)-7-cyano-3-methyl-pyrrolo[2,3-c]pyridin-4-yl)pyrimidine

To a solution of (1,N-(tert-butoxycarbonyl)-7-cyano-3-methyl-1H-pyrrolo[2,3-c]pyridin-5-yl)boronic acid (200 mg, 0.66 mmol, 1.0 eq) in dioxane (5 mL) and water (1 mL) was added 2,4-dichloropyrimidine (109 mg, 0.73 mmol, 1.1 eq), Pd(PPh₃)₄ (76 mg, 0.066 mmol, 0.1 eq) and K₂CO₃ (273 mg, 1.98 mmol, 3.0 eq). The mixture was purged with nitrogen three times, then heated at 100° C. under nitrogen for 3 hours. After TLC and LCMS indicated completion, the mixture was filtered, the filtrate was concentrated and purified on silica column affording 2-chloro-4-(1,N-(tert-Butoxycarbonyl)-7-cyano-3-methyl-pyrrolo[2,3-c]pyridin-4-yl) pyrimidine (90 mg, 36%). ESI-MS (m/z): 269.9 (M+H−100)⁺.

### A4. 2-Chloro-4-(6-cyano-1-methyl-1H-indol-4-yl) pyrimidine

### 3-Bromo-4-methyl-5-nitrobenzoic Acid

To a mixture of 4-methyl-3-nitrobenzoic acid (25.0 g, 138 mmol, 1.0 eq) in conc. H₂SO₄ (100 mL) was added 1,3-dibromo-5,5-dimethylimidazolidine-2,4-dione (39.4 g, 152 mmol, 1.1 eq) portion wise at room temperature. When the addition was completed, the reaction mixture was stirred at room temperature overnight. The reaction mixture was poured into ice-water (500 g) with stirring, the white solid was formed, filtered and dried in vacuo to give 3-bromo-4-methyl-5-nitrobenzoic acid (32 g, 89%). ¹HNMR (300 MHz, DMSO-d₆): δ 8.33-8.31 (m, 2H), 2.72 (s, 3H). ESI-MS (m/z): 257.7 (M−H)⁻.

### Methyl 3-bromo-4-methyl-5-nitrobenzoate

To a solution of 3-bromo-4-methyl-5-nitrobenzoic acid (32.0 g, 123 mmol, 1.0 eq) in MeOH (1.2 L) at room temperature was added conc. H₂SO₄ (5 mL), the mixture was heated to reflux and stirred for 8 h. After TLC and LCMS indicated completion, the mixture was concentrated under reduced pressure to remove most of MeOH, diluted with EtOAc (200 mL), washed with sat. NaHCO₃ (100 mL×2), the organic layer was dried over Na₂SO₄, concentrated in vacuo to obtain methyl 3-bromo-4-methyl-5-nitrobenzoate, which was used without further purification in the next step as a white solid (30 g, crude). ¹H NMR (300 MHz, DMSO-d₆): δ 8.36 (s, 2H), 3.90 (s, 3H), 2.54 (s, 3H).

### Methyl 4-bromo-1H-indole-6-carboxylate

A solution of methyl 3-bromo-4-methyl-5-nitrobenzoate (20.0 g, 73.0 mmol, 1.0 eq) and DMF-DMA (19.5 mL, 146.0 mmol, 2.0 eq) in dry DMF (100 ml) was heated to 120° C. and stirred for 6 h. After cooling to 25° C., the reaction was concentrated under reduced pressure to dryness. The residue was dissolved in AcOH (250 mL), Fe powder (82 g) was added to the mixture with stirring. The mixture was heated to 100° C. and stirred overnight, cooled to room temperature, diluted with EtOAc and filtered by celite, extracted with EtOAc (500 mL), and washed with sat. NaHCO₃, then with water and brine. The organic layer was dried over anhydrous Na₂SO₄ and concentrated to give an orange solid. Further purification by column chromatography (SiO₂, 100-200 m, eluted by n-hexane/EtOAc=20:1 to 10:1) to provide methyl 4-bromo-1H-indole-6-carboxylate. (8.0 g, 42%, two steps) as a yellow solid. ¹H NMR (300 MHz, DMSO-d₆): δ 11.92 (br, 1H), 8.08 (s, 1H), 7.80-7.70 (m, 2H), 6.50 (sl br s, 1H), 3.87 (s, 3H). ESI-MS (m/z): 253.7 (M+H)⁺.

### 4-Bromo-1-methyl-1H-indole-6-carboxylic Acid

To a solution of methyl 4-bromo-1H-indole-6-carboxylate (8.06 g, 30.6 mmol, 1.0 eq) in DMF (50 mL) at 0° C. was added NaH (60% in mineral oil, 1.8 g, 45.9 mmol, 1.5 eq) portion wise. The stirring mixture was allowed to warm to room temperature and stirred for 10 min. Re-cooled to 0° C. and then MeI (6.5 g, 45.9 mmol, 1.5 eq) was added drop wise. The reaction mixture was stirred at room temperature for 1 h, poured into 0.5N HCl (30 mL), extracted with EtOAc (50 mL×2), washed with water (50 mL), brine (50 mL) and dried over sodium sulfate. After filtration and removal of the solvent, the residue was dissolved in MeOH (30 mL) and THF (30 mL). NaOH (3M, 30 mL) was added, the mixture was stirred at room temperature for 2 h, LCMS showed no starting materials left, the residue was diluted with H₂O (50 mL), washed with EtOAc (25 mL×1), the hydrous layer was neutralized by 1N HCl to pH 3-4, the solid formed was filtered and dried to afford 4-bromo-1-methyl-1H-indole-6-carboxylic acid. (7.5 g, 57%) as a white solid. H NMR (300 MHz, DMSO-d₆): δ 13.1-12.8 (brs, 1H), 8.11 (s, 1H), 7.78 (s, 1H), 7.69 (d, J=2.1 Hz, 1H), 6.47 (sl brs, 1H), 3.89 (s, 3H).

### 4-Bromo-1-methyl-1H-indole-6-carboxamide

To a solution of 4-bromo-1-methyl-1H-indole-6-carboxylic acid (3.5 g, 13.8 mmol, 1.0 eq) and a drop of DMF (cat.) in DCM (50 mL) at 0° C. was added oxalyl chloride (2.4 mL, 27.6 mmol, 2.0 eq) drop wise. When the addition was completed, the reaction mixture was warmed to room temperature with stirring for 3 h, concentrated in vacuo to dryness. The crude the acyl chloride was dissolved in dry THF (20 mL) and was added to a mixture of concentrated aqueous ammonia (10 mL) and THF (20 mL) drop wise at 0° C. When the addition was completed, the reaction mixture was stirred at rt for 1 h, extracted by EtOAc, washed by brine, dried over Na₂SO₄, filtered and purified by column chromatography to afford 4-bromo-1-methyl-1H-indole-6-carboxamide as white solid (2.5 g, 71.4%). ¹H NMR (300 MHz, DMSO-d₆): δ 8.11 (s, 1H), 8.03 (brs, 1H), 7.81 (s, 1H), 7.62 (s, 1H), 7.37 (brs, 1H), 6.43 (s, 1H), 3.87 (s, 3H). ESI-MS (m/z): 252.8 (M+H)⁺.

### 4-Bromo-6-cyano-1-methyl-1H-indole

To a solution of 4-bromo-1-methyl-1H-indole-6-carboxamide (2.5 g, 9.8 mmol, 1.0 eq)) in toluene (50 mL) was added POCl₃ (0.8 mL) drop wise. When the addition was completed, the reaction mixture was heated to reflux and stirred for 3 h. Cooled to room temperature, poured into ice-water slowly, extracted with EtOAc (50 mL×2), washed by sat. NaHCO₃ (20 mL) and brine, concentrated in vacuo to afford the crude product. Further purification by column chromatography gave 4-bromo-6-cyano-1-methyl-1H-indole as a yellow solid (1.4 g, 60%). ¹H NMR (300 MHz, DMSO-d₆): δ 8.18 (s, 1H), 7.78 (sl brs, 1H), 7.67 (s, 1H), 6.53 (sl br s, 1H), 3.89 (s, 3H).

### 6-Cyano-1-methyl-4-(4,4,5,5-tetramethyl-1,3,2-dioxaborolan-2-yl)-1H-indole

A solution of 4-bromo-6-cyano-1-methyl-1H-indole (1.4 g, 6.0 mmol, 1.0 eq), bis(pinacolato)diboron (2.28 g, 9 mmol, 1.5 eq), Pd(dppf)Cl₂ (219 mg, 0.3 mmol, 0.05 eq)) and KOAc (1.18 g, 12 mmol, 2.0 eq) in dioxane (25 mL) was heated to reflux and stirred for 1 h under N₂ atmosphere. As LCMS showed no starting materials left, the reaction mixture was filtered through celite, and purified by column chromatography to afford 1-methyl-6-cyano-4-(4,4,5,5-tetramethyl-1,3,2-dioxaborolan-2-yl)-1H-indole as a yellow solid (1.5 g, 89%). ¹H NMR (300 MHz, DMSO-d₆): δ 8.25 (s, 1H), 7.78 (sl br s, 1H), 7.67 (s, 1H), 6.86 (sl br s, 1H), 3.89 (s, 3H), 1.30 (s, 12H).

### 2-Chloro-4-(6-cyano-1-methyl-1H-indol-4-yl)pyrimidine

A mixture of 1-methyl-6-cyano-4-(4,4,5,5-tetramethyl-1,3,2-dioxaborolan-2-yl)-1H-indole (1.5 g, 5.3 mmol, 1.0 eq), 2,4-dichloropyrimidine (790 mg, 5.3 mmol, 1.0 eq), Pd(dppf)Cl₂ (193 mg, 0.26 mmol, 0.05 eq) and K₂CO₃ (1.45 g, 10.6 mmol, 2.0 eq) in dioxane (25 mL) and H₂O (5 mL) was heated to 80° C. and stirred for 2 h under N₂ atmosphere. As LCMS showed no starting materials left, the reaction mixture was filtered through celite, concentrated in vacuo to afford the crude product, and further purified by column chromatography to afford 2-chloro-4-(6-cyano-1-methyl-1H-indol-4-yl)pyrimidine as a yellow solid (800 mg, 56%). ¹H NMR (300 MHz, DMSO-d₆): δ 8.87 (d, J=5.1 Hz, 1H), 8.36 (s, 1H), 8.26 (d, J=5.1 Hz, 1H), 8.19 (s, 1H), 7.87 (s, 1H), 7.26 (s, 1H), 3.95 (s, 3H). ESI-MS (m/z): 268.9 (M+H)⁺.

### A5. 2-Chloro-4-(3-(N,N-dimethylamino)-1H-thieno[2,3-c]pyrazol-1-yl)pyrimidine

### N′-(2-bromothiophene-3-carbonyl)-4-methylbenzenesulfonohydrazide

2-Bromothiophene-3-carboxylic acid (10.3 g, 50 mmol, 1.0 eq) was stirred in DCM (125 mL) with a drop of NMP. Thionyl chloride (7.1 g, 60 mmol, 1.2 eq) was added and the reaction heated to reflux for 2 hours. The solvent was removed at reduced pressure to afford a crude residue (11 g). The residue was dissolved in toluene (250 mL) and 4-methylbenzene sulfonohydrazide (18.6 g, 100 mmol, 2.0 eq) was added, the mixture was heated to 1000 for 2 hours. The reaction mixture was allowed to cool to room temperature and the suspension filtered. The solid was slurried with 1N HCl and the suspension filtered. The solid was washed with water and dried in vacuo at 40° C. overnight to afford N′-(2-bromothiophene-3-carbonyl)-4-methylbenzenesulfonohydrazide (11.0 g, 73%). ¹HNMR (300 MHz, DMSO-d₆): δ 10.48 (s, 1H), 9.97 (s, 1H), 7.75 (d, J=8.1 Hz, 2H), 7.62 (d, J=5.4 Hz, 1H), 7.35 (d, J=8.1 Hz, 2H), 7.06 (d, J=5.4 Hz, 1H), 2.36 (s, 3H). ESI-MS (m/z): 374.7 (M+H)⁺.

### 2-Bromo-N,N-dimethyl-N′-tosylthiophene-3-carboamidrazone

N′-(2-bromothiophene-3-carbonyl)-4-methylbenzenesulfonohydrazide (10 g, 26.7 mmol, 1.0 eq) was heated to 80° C. in thionyl chloride (18.9 g, 160 mmol, 6.0 eq) for 1 hour. The reaction mixture was allowed to cool to room temperature and concentrated in vacuo to give a crude residue. The residue was dissolved in THF (150 mL) at 0° C. and DABCO (5.98 g, 53.4 mmol, 2.0 eq) was added, then a solution of dimethylamine in THF (53.4 mL) was added dropwise. The reaction was warmed to room temperature and stirred overnight. The reaction was concentrated in vacuo to remove the solvent and water (200 mL) was added, extracted with DCM (150 mL×3). The combined organic layers were dried over anhydrous sodium sulfate and concentrated in vacuo to give a crude residue, which was purified by silica gel chromatography to give 2-Bromo-N,N-dimethyl-N′-tosylthiophene-3-carboamidrazone as yellow solid (4 g, 37%). ¹H NMR (300 MHz, DMSO-d₆): δ 8.75 (s, 1H), 7.71-7.68 (m, 3H), 7.37 (d, J=7.5 Hz, 2H), 6.85 (d, J=5.1 Hz, 1H), 2.68 (s, 6H), 2.38 (s, 3H). ESI-MS (m/z): 401.7 (M+H)⁺.

### 3-(N,N-dimethylamino)-1-tosyl-1H-thieno[2,3-c]pyrazole

A mixture of 2-bromo-N,N-dimethyl-N′-tosylthiophene-3-carboamidrazone (1.0 g, 2.5 mmol, 1.0 eq), CuI (95 mg, 0.5 mmol, 0.2 eq), K₂CO₃ (690 mg, 5 mmol, 2.0 eq) in NMP (10 mL) was heated to 110° C. in a microwave for 20 min. After LCMS indicated completion, the reaction mixture was poured on to water (10 mL) and filtered, the filter cake was washed with water, and dried to give the title 3-(N,N-dimethylamino)-1-tosyl-1H-thieno[2,3-c]pyrazole (410 mg, 51%). ¹HNMR (300 MHz, DMSO-d₆): δ 7.67 (d, J=8.1 Hz, 2H), 7.37 (2H, d J=8.1 Hz-7.33 (d, J=5.1 Hz, 1H), 7.18 (d, J=5.1 Hz, 1H), 2.95 (s, 6H), 2.33 (s, 3H). ESI-MS (m/z): 321.8 (M+H)⁺.

### 3-(N,N-dimethylamino)-1H-thieno[2,3-c]pyrazole

3-(N,N-dimethylamino)-1-tosyl-1H-thieno[2,3-c]pyrazole (1.28 g, 4 mmol, 1.0 eq) and potassium hydroxide (1.12 g, 20 mmol, 5.0 eq) were combined in methanol (40 mL) and heated to reflux for 30 minutes. The solvent was removed at reduced pressure. The resulting residue was taken up in DCM (400 mL) washed with water (250 mL), dried over sodium sulfate and concentrated at reduced pressure. The residue was purified by silica gel chromatography to give 3-(N,N-dimethylamino)-1H-thieno[2,3-c]pyrazole (180 mg, 27%). ¹H NMR (300 MHz, CDCl₃): δ 8.83 (br, 1H), 6.95 (d, J=2.4 Hz, 1H), 6.74 (d, J=2.4 Hz, 1H), 3.08 (s, 6H). ESI-MS (m/z): 167.9 (M+H)⁺.

### 2-Chloro-4-(3-(N,N-dimethylamino)-1H-thieno[2,3-c]pyrazol-1-yl)pyrimidine

To a solution of 3-(N,N-dimethylamino)-1H-thieno[2,3-c]pyrazole (180 mg, 1.08 mmol, 1.0 eq) in DMF (2 mL) was added t-BuOK (181 mg, 1.62 mmol, 1.5 eq) at 0° C. The mixture was stirred at this temperature for 30 minutes, then a solution of 2,4-dichloropyrimidine (192 mg, 1.3 mmol, 1.2 eq) was added. The mixture was stirred at RT overnight. After completion, the mixture was quenched with aq. sat. NH₄Cl (4 mL) and then diluted with water (4 mL), extracted with EA (5 mL×3). The combined organic layers were washed with water (10 mL), concentrated and purified by column chromatography on silica to give 2-chloro-4-(3-(N,N-dimethylamino)-1H-thieno[2,3-c]pyrazol-1-yl)pyrimidine (80 mg, 27%). ¹H NMR (300 MHz, CDCl₃): δ 8.41 (d, J=4.2 Hz, 1H), 7.52 (d, J=4.2 Hz, 1H), 7.15-7.00 (m, 2H), 3.18 (s, 6H). ESI-MS (m/z): 279.8 (M+H)⁺.

### A6. 2-Chloro-4-(3-(N,N-dimethylamino)-5-chloro-1H-thieno[2,3-c]pyrazol-1-yl)pyrimidine

To a solution of 2-chloro-4-(3-(N,N-dimethylamino)-1H-thieno[2,3-c]pyrazol-1-yl)pyrimidine (140 mg, 0.5 mmol, 1.0 eq) in a mixed solution of benzene and acetic acid (1:1, 1.4 mL) was added NCS (73.4 mg, 0.55 mmol, 1.1 eq). The mixture was heated to 70° C. and stirred for 2 hours. After completion, the mixture was poured into ice water (5 g), extracted with DCM (5 mL×2), the combined organic layers were washed with brine (5 mL), dried, concentrated and purified by silica column to give 2-chloro-4-(3-(N,N-dimethylamino)-5-chloro-1H-thieno[2,3-c]pyrazol-1-yl)pyrimidine (78.3 mg, 50%). ¹H NMR (300 MHz, CDCl₃): δ 8.45 (d, J=5.4 Hz, 1H), 7.52 (d, J=5.4 Hz, 1H), 7.01 (s, 1H), 3.15 (s, 6H). ESI-MS (m/z): 314.0 (M+H)⁺.

### A7. 2-Chloro-4-(4-cyano-1-methyl-1H-indol-6-yl)pyrimidine 5-Bromo-2-methyl-3-nitrobenzoic Acid

1,3-Dibromo-5,5-Dimethylimidazolidine-2,4-dione (47.2 g, 166 mmol, 1.0 eq) was added portionwise to a stirred mixture of 2-methyl-3-nitrobenzoic acid (30 g, 166 mmol, 1.0 eq) in conc. H₂SO₄ (100 mL) at room temperature. When the addition was completed, the reaction mixture was stirred at room temperature overnight. The reaction mixture was poured into ice-water (500 g) with stirring, forming a white solid which was filtered and dried in vacuo to give the desired product 5-bromo-2-methyl-3-nitrobenzoic acid (35 g, 81%). ¹H NMR (300 MHz, DMSO-d₆): δ 8.30 (s, 1H), 8.14 (s, 1H), 2.44 (s, 3H).

### Methyl 5-bromo-2-methyl-3-nitrobenzoate

To a solution of 5-bromo-2-methyl-3-nitrobenzoic acid (35 g, 135 mmol, 1.0 eq) in MeOH (1.2 L) at room temperature was added conc. H₂SO₄ (5 mL), the mixture was heated to reflux and stirred for 8 h. LCMS showed no starting materials left. It was concentrated under reduced pressure to remove most MeOH, diluted with EtOAc (200 mL), washed with sat. NaHCO₃ (100 mL×2), the organic layer was dried over Na₂SO₄, and concentrated in vacuo to give the desired product methyl 5-bromo-2-methyl-3-nitrobenzoate (30 g, crude), which was used directly in the next step without further purification. ¹H NMR (300 MHz, DMSO-d₆): δ 8.14 (s, 1H), 8.00 (s, 1H), 3.96 (s, 3H), 2.58 (s, 3H).

### Methyl 6-bromo-1H-indole-4-carboxylate

A solution of 5-bromo-2-methyl-3-nitrobenzoate (40 g, 146 mmol, 1.0 eq) and DMF-DMA (39 mL, 292.0 mmol, 2.0 eq) in DMF (150 ml) was heated to 120° C. for 6 h. After cooling to 25° C., the reaction was concentrated under reduced pressure to dryness. The residue was dissolved in AcOH (350 mL), Fe powder (164 g) was added to the mixture by portions with vigorous stirring by portions. The mixture was heated to 100° C. and stirred overnight. Cooled to room temperature, diluted with EtOAc and filtered by celite, extracted with EtOAc (500 mL), and washed with sat. NaHCO₃, then with water and brine. The organic layer was dried over anhydrous Na₂SO₄ and concentrated to give an orange solid as a crude residue. Further purification by column chromatography (SiO₂, 100-200 m, eluted by n-hexane/EtOAc=20:1 to 10:1) provided methyl 6-bromo-1H-indole-4-carboxylate (22.0 g, 59%, two steps) as a yellow solid. ¹H NMR (300 MHz, DMSO-d₆): δ 8.46 (br, 1H), 8.04 (s, 1H), 7.75 (s, 1H), 7.36 (s, 1H), 7.18 (s, 1H), 4.01 (s, 3H). ESI-MS (m/z): 253.8 (M+H)⁺.

### 6-Bromo-1-methyl-1H-indole-4-carboxylic acid

To a solution of NaH (60% in mineral oil, 2.23 g, 56.9 mmol, 1.5 eq) in DMF (50 mL) at 0° C. was added methyl 6-bromo-1H-indole-4-carboxylate (10.0 g, 37.9 mmol, 1.0 eq). The mixture was allowed to warm to room temperature and stirred for 10 min. After re-cooling to 0° C., MeI (8.06 g, 56.9 mmol, 1.5 eq) was added drop wise. The reaction mixture was stirred at room temperature for 1 h and was quenched by 0.5 N HCl (30 mL), extracted by EtOAc (70 mL×2), washed with water (1×50 mL), brine and dried over sodium sulfate. After filtration and removal of the solvent, the residue was dissolved in MeOH (40 mL) and THF (40 mL), 3 N NaOH (40 mL) was added, the mixture was stirred at room temperature for 2 h, when LC-MS showed no starting materials left. The residue was diluted with H₂O (50 mL), and washed with EtOAc (35 mL). When the aqueous layer was neutralized by 1 N HCl to pH=3-4, a solid was formed, which was filtered and dried in vacuo to afford the desired product 6-bromo-1-methyl-1H-indole-4-carboxylic acid (7.6 g, 79%) as a white solid. ¹H NMR (300 MHz, DMSO-d₆): δ 8.00 (s, 1H), 7.78 (s, 1H), 7.51 (s, 1H), 6.92 (s, 1H), 3.37 (s, 3H).

### 6-Bromo-1-methyl-1H-indole-4-carboxamide

To a solution of 6-bromo-1-methyl-1H-indole-4-carboxylic acid (4.5 g, 17.7 mmol, 1.0 eq) and a drop of DMF (cat.) in DCM (60 mL) at 0° C. was added oxalyl dichloride (3.1 mL, 35.4 mmol, 2.0 eq) drop wise. When the addition was completed, the reaction mixture was warmed to room temperature and stirred for 3 h, then concentrated in vacuo to dryness. The residue was dissolved in dry THF (30 mL) and was added to a mixture of concentrated aqueous ammonia (15 mL) and THF (30 mL) drop wise at 0° C. When the addition was completed, the reaction mixture was stirred at room temperature for 1 h, extracted by EtOAc, washed with brine, dried over Na₂SO₄, filtered and concentrated to give a crude residue, which was purified by column chromatography to afford the desired product 6-bromo-1-methyl-1H-indole-4-carboxamide as white solid (1.2 g, 22%). ¹H NMR (300 MHz, CDCL₃): δ 8.76 (d, J=3.0 Hz, 1H), 8.50 (d, J=8.4 Hz, 1H), 8.37 (s, 1H), 7.87 (s, 1H), 7.51-7.48 (m, 1H), 7.10 (s, 1H), 3.89 (s, 3H). ESI-MS (m/z): 253.0 (M+H)⁺.

### 6-Bromo-4-cyano-1-methyl-1H-indole

To a solution of 6-bromo-1-methyl-1H-indole-4-carboxamide (1.2 g, 4.9 mmol, 1.0 eq) in toluene (50 mL) was added POCl₃ (0.4 mL) drop wise, when the addition was completed, the reaction mixture was heated to reflux and stirred for 3 h. Cooled to room temperature, poured into ice cold-water slowly, extracted with EtOAc (50 mL×2), washed with sat. NaHCO₃ (20 mL) and brine, dried (Na₂SO₄) and concentrated in vacuo to afford the crude product, which was further purified by column chromatography to give the desired product 6-bromo-4-cyano-1-methyl-1H-indole as a yellow solid (0.46 g, 41%). ESI-MS (m/z): 236.6 (M+H)⁺.

### 4-Cyano-1-methyl-6-(4,4,5,5-tetramethyl-1,3,2-dioxaborolan-2-yl)-1H-indole

A solution of 6-bromo-4-cyano-1-methyl-1H-indole (0.46 g, 2.0 mmol, 1.0 eq), bis(pinacolato)diboron (0.75 g, 3.0 mmol, 1.5 eq), Pd(dppf)Cl₂ (72 mg, 0.09 mmol, 0.05 eq) and KOAc (0.39 g, 4.0 mmol, 2.0 eq) in dioxane (25 mL) was heated to reflux under N₂ for 1 h, LCMS showed no starting materials left, the reaction mixture was filtered though celite, concentrated under reduced pressure, and purified by column chromatography to afford the desired product 4-cyano-1-methyl-6-(4,4,5,5-tetramethyl-1,3,2-dioxaborolan-2-yl)-1H-indole as a yellow solid (0.52 g, 93%). ¹H NMR (300 MHz, DMSO-d₆): δ 8.02 (s, 1H), 7.94 (s, 1H), 7.28 (s, 1H), 6.71 (s, 1H), 3.90 (s, 3H), 1.40 (s, 12H).

### 2-Chloro-4-(4-cyano-1-methyl-1H-indol-6-yl)pyrimidine

A slurry of 4-cyano-1-methyl-6-(4,4,5,5-tetramethyl-1,3,2-dioxaborolan-2-yl)-1H-indole (0.5 g, 1.6 mmol, 1.0 eq), 2,4-dichloropyrimidine (263 mg, 1.6 mmol, 1.0 eq), Pd(dppf)Cl₂ (64 mg, 0.09 mmol, 0.05 eq) and K₂CO₃ (0.48 g, 3.53 mmol, 2.0 eq) in dioxane (10 mL) and H₂O (2 mL) was heated to 80° C. and stirred for 2 h under N₂. LCMS showed no starting materials left, so the reaction mixture was filtered through celite, and. concentrated in vacuo to afford the crude product. This was further purified by column chromatography to afford the desired product 2-chloro-4-(4-cyano-1-methyl-1H-indol-6-yl)pyrimidine as a yellow solid (300 mg, 70%). ESI-MS (m/z): 268.5 (M+H)⁺.

### A8. 2-Chloro-4-(3-methoxy-1H-indazol-1-yl) pyrimidine

### 1-N-Ethoxycarbonyl-3-hydroxy-1H-indazole

Ethyl chloroformate (8.9 g, 82 mmol, 1.1 eq) was slowly added to a suspension of 3-hydroxy-1H-indazole (10 g, 74.6 mmol, 1.0 eq) in pyridine (50 mL), the reaction mixture was heated to 100° C. and stirred 5 h. TLC, LC-MS indicated starting material disappeared and then poured into water (400 mL) and the precipitate was collected by filtration, washed with water (200 mL) and acetone (350 mL), and then air dried to give 1-N-ethoxycarbonyl-3-hydroxy-1H-indazole (13.0 g, 86%). ¹H NMR (300 MHz, DMSO-d₆) δ 12.19 (br, 1H), 8.04 (d, J=8.4 Hz, 1H), 7.73 (d, J=8.4 Hz, 1H), 7.61 (t, J=7.8 Hz, 1H), 7.34 (t, J=7.8 Hz, 1H), 4.40 (q, J=6.6 Hz, 2H), 1.36 (t, J=7.2 Hz, 3H). ESI-MS (m/z): 207.1 (M+H)⁺.

### 1-N-Ethoxycarbonyl-3-methoxy-1H-indazole

To a solution of 1-N-ethoxy carbonyl-3-hydroxy-1H-indazole (5.0 g, 24.2 mmol, 1.0 eq) in acetone (50 mL) was added Cs₂CO₃ (9.5 g, 29.1 mmol, 1.2 eq) and iodomethane (4.13 g, 29.1 mmol, 1.2 eq), then heated to 70° C. and stirred for 2 h. LC-MS indicated starting material had disappeared. The reaction mixture was filtered, the precipitate rinsed with EA (50 mL), and the combined filtrates were concentrated under reduced pressure to give a crude residue, which was purified by silica gel column chromatography to give 1-N-ethoxycarbonyl-3-methoxy-1H-indazole (1.9 g, 34%). ¹H NMR (300 MHz, CDCl₃): δ 8.20-8.05 (m, 1H), 7.68 (d, J=7.8 Hz, 1H), 7.56 (t, J=7.2 Hz, 1H), 7.31 (t, J=7.2 Hz, 1H), 4.58 (q, J=7.2 Hz, 2H), 4.21 (s, 3H), 1.52 (t, J=7.2 Hz, 3H). ESI-MS (m/z): 221.1 (M+H)⁺.

### 3-Methoxy-1H-indazole

To a solution of 1-N-ethoxycarbonyl-3-methoxy-1H-indazole (1.9 g, 8.6 mmol, 1.0 eq) in EtOH (50 mL) was added 1N NaOH(aq) (12.9 mL). The reaction was stirred at room temperature for 2 h when LC-MS indicated starting material was gone. The reaction mixture was extracted with EA (50 mL×3) and the organic extract was washed with brine (30 mL), dried over sodium sulfate, and concentrated under reduced pressure, affording the desired product 3-methoxy-1H-indazole (1.2 g, 93%). ¹H NMR (300 MHz, DMSO-d₆) δ 11.91 (br, 1H), 7.58 (d, J=7.5 Hz, 1H), 7.45-7.34 (m, 2H), 7.00 (t, J=6.0 Hz, 1H), 3.99 (s, 3H). ESI-MS (m/z): 149.1 (M+H)⁺.

### 2-Chloro-4-(3-methoxy-1H-indazol-1-yl) pyrimidine

A solution of 3-methoxy-1H-indazole (1.2 g, 8.1 mmol, 1.0 eq) in DMF (24 mL) was cooled to 0° C., and t-BuOK (1.0 g, 8.9 mmol, 1.1 eq) was added carefully. The mixture was stirred at this temperature for 10 minutes. Then a solution of 2,4-dichloropyrimidine (1.27 g, 8.1 mmol, 1.0 eq) in DMF (10 mL) was added drop wise. The mixture was stirred at RT for 2 h. After completion, the mixture was diluted with water (80 mL), filtered, the filter cake was washed with water (10 mL×2), dried and then was purified by column chromatography on silica to give 2-chloro-4-(3-methoxy-1H-indazol-1-yl) pyrimidine (1.2 g, 57%) as an off-white solid.

¹H NMR (300 MHz, DMSO-d₆) δ 8.63 (d, J=5.4 Hz, 1H), 8.54 (d, J=8.4 Hz, 1H), 7.83-7.67 (m, 3H), 7.41 (t, J=7.5 Hz, 1H), 4.16 (s, 3H). ESI-MS (m/z): 261.0 (M+H)⁺.

### A9. 2-Chloro-4-(6-cyano-1-methyl-1H-indazol-4-yl)pyrimidine

### 3-Bromo-4-methyl-5-nitrobenzoic acid

To a mixture of 4-methyl-3-nitrobenzoic Acid (36.0 g, 200 mmol, 1.0 eq) in conc. H₂SO₄ (150 mL) was added 1,3-dibromo-5,5,-dimethylhydantoin (51.8 g, 200 mmol, 1.0 eq) portion wise at room temperature. When the addition was completed, the reaction mixture was stirred at room temperature overnight. The reaction mixture was poured into ice-water (500 g) with stirring, the white precipitates solid was filtered and dried in vacuo to give the desired product 3-bromo-4-methyl-5-nitrobenzoic acid (40 g, 77%). ¹H NMR (300 MHz, DMSO-d₆): δ 8.33 (s, 1H), 8.31 (s, 1H), 2.53 (s, 3H). ESI-MS (m/z): 257.9 (M−H)⁻.

### Methyl 3-bromo-4-methyl-5-nitrobenzoate

To a solution of 3-bromo-4-methyl-5-nitrobenzoic acid (40.0 g, 153 mmol, 1.0 eq) in MeOH (1.2 L) at room temperature was added conc. H₂SO₄ (10 mL), the mixture was heated to reflux and stirred for 8 h. LCMS showed no starting materials left. The mixture was concentrated under reduced pressure to remove most of MeOH, diluted with EtOAc (200 mL), washed with sat. NaHCO₃ (100 mL×2), the organic layer was dried over Na₂SO₄, concentrated in vacuo to give the desired product methyl 3-bromo-4-methyl-5-nitrobenzoate (40 g, 93%) as a white solid, which was used in the next step without further purification. ¹H NMR (300 MHz, DMSO-d₆): δ 8.35 (s, 2H), 3.90 (s, 3H), 2.50 (s, 3H).

### Methyl 3-amino-5-bromo-4-methylbenzoate

To a solution of methyl 3-bromo-4-methyl-5-nitrobenzoate (20 g, 73.0 mmol, 1.0 eq) in EtOH (150 ml) and AcOH (50 mL) was added Fe powder (16.3 g, 292 mmol, 4.0 eq) portion wise. The mixture was heated to reflux and stirred for 2 h. LCMS indicated starting material disappeared, the reaction mixture was cooled to room temperature, diluted with EtOAc (200 mL) and filtered by celite, washed with sat. NaHCO₃, water and brine. The organic layer was dried over anhydrous Na₂SO₄ and concentrated in vacuo to give methyl 3-amino-5-bromo-4-methylbenzoate (13 g, 73%) as a white solid. ESI-MS (m/z): 244.0 (M+H)⁺.

### Methyl 4-bromo-1H-indazole-6-carboxylate

To a solution of methyl 3-amino-5-bromo-4-methylbenzoate (13 g, 53.5 mmol, 1.0 eq) in AcOH (150 mL) at 50C was added aq. NaNO₂ (3.7 g, 5 mL, 53.5 mmol, 1.0 eq) drop wise. The stirring mixture was allowed to warm to room temperature and stirred overnight, the reaction mixture was poured into ice-water, the precipitates solid was filtered and dried to give a crude residue, which was purified by column chromatography to give methyl 4-bromo-1H-indazole-6-carboxylate (4.5 g, 33%). ¹H NMR (300 MHz, DMSO-d₆): δ 8.17 (s, 2H), 7.80 (s, 1H), 3.90 (s, 3H). ESI-MS (m/z): 255.0 (M+H)⁺.

### Methyl 4-bromo-1-methyl-1H-indazole-6-carboxylate

To a solution of methyl 4-bromo-1H-indazole-6-carboxylate (4.5 g, 17.6 mmol, 1.0 eq) in DMF (50 mL) at 0° C. was added NaH (60% in mineral oil, 1.0 g, 26.4 mmol, 1.5 eq) portion wise. The stirring mixture was allowed to warm to room temperature and stirred for 10 min. Re-cooled to 0° C. and then MeI (3.7 g, 26.4 mmol, 1.5 eq) was added drop wise. The reaction mixture was stirred at room temperature for 1 h, poured into 0.5N HCl (30 mL), extracted with EtOAc (50 mL×2), washed with water (50 mL), brine (50 mL) and dried over sodium sulfate. The residue was purified by column chromatography to give methyl 4-bromo-1-methyl-1H-indazole-6-carboxylate (2.5 g, 53%). ¹H NMR (300 MHz, DMSO-d₆): δ 8.36 (s, 1H), 8.15 (s, 1H), 7.83 (s, 1H), 4.17 (s, 3H), 3.92 (s, 3H). ESI-MS (m/z): 269.0 (M+H)⁺.

### 4-Bromo-1-methyl-1H-indazole-6-carboxylic Acid

To a solution of methyl 4-bromo-1-methyl-1H-indazole-6-carboxylate (2.5 g, 9.3 mmol, 1.0 eq) in THF (15 mL) and MeOH (15 mL) was added aq. NaOH (12 mL, 37 mmol, 3 N), and the reaction mixture was stirred at room temperature for 1 h. LCMS showed no starting materials left. The reaction mixture was concentrated under reduced pressure to remove most of THF and MeOH, the residue was diluted with H₂O, neutralized by 1N. HCl to pH=3-4, the solid precipitates were filtered and dried to afford the desired product 4-bromo-1-methyl-1H-indazole-6-carboxylic acid (1.8 g, 76%). ¹HNMR (300 MHz, DMSO-d₆): δ 8.32 (s, 1H), 8.12 (s, 1H), 7.82 (s, 1H), 4.15 (s, 3H). ESI-MS (m/z): 254.9 (M+H)⁺.

### 4-Bromo-1-methyl-1H-indazole-6-carboxamide

To a solution of 4-bromo-1-methyl-1H-indazole-6-carboxylic acid (1.8 g, 7.1 mmol, 1.0 eq) and a drop of DMF (cat.) in DCM (50 mL) at 0° C. was added oxalyl chloride (1.5 mL, 17 mmol, 7.5 eq) drop wise, when the addition was completed, the reaction mixture was heated to reflux for 3 h. The solution was cooled to room temperature, concentrated in vacuo to dryness, and the acyl chloride was dissolved in dry THF (20 mL) and was added drop wise to a mixture of concentrated aqueous ammonia (10 mL) and THF (20 mL) at 0° C. When the addition was completed, the reaction mixture was stirred at room temperature for 1 h, extracted with EtOAc, washed by brine, dried over Na₂SO₄, filtered and concentrated in vacuo to afford the desired product 4-bromo-1-methyl-1H-indazole-6-carboxamide as white solid (1.8 g, crude). ESI-MS (m/z): 253.9 (M+H)⁺.

### 4-Bromo-6-cyano-1-methyl-1H-indazole

To a mixture of 4-bromo-1-methyl-1H-indazole-6-carboxamide (1.8 g, 7.1 mmol, 1.0 eq) in toluene (50 mL) was added POCl₃ (0.8 mL) drop wise, when the addition was completed, the reaction mixture was heated to reflux and stirred for 3 h. After cooling to room temperature, the reaction mixture was poured into ice-water slowly, extracted with EtOAc (50 mL×2), the combined organic layers were washed with sat. NaHCO₃ (20 mL) and brine, dried and concentrated in vacuo to afford the crude product, which was further purified by column chromatography to give the desired product 4-bromo-6-cyano-1-methyl-1H-indazole as a yellow solid (1.0 g, 63%). ¹H NMR (300 MHz, DMSO-d₆): δ 8.48 (s, 1H), 8.20 (s, 1H), 7.80 (s, 1H), 4.14 (s, 3H). ESI-MS (m/z): 236.0 (M+H)⁺.

### 6-Cyano-1-methyl-4-(4,4,5,5-tetramethyl-1,3,2-dioxaborolan-2-yl)-1H-indazole

A solution of 4-bromo-6-cyano-1-methyl-1H-indazole (1.0 g, 4.2 mmol, 1.0 eq), bis(pinacolato)diboron (2.1 g, 8.4 mmol, 2.0 eq), Pd(dppf)Cl₂ (154 mg, 0.21 mmol, 0.05 eq) and KOAc (0.8 g, 8.4 mmol, 2.0 eq) in dioxane (25 mL) were heated to reflux and stirred for 1 h under N₂ atmosphere. As LCMS showed no starting materials left, the reaction mixture was filtered through celite, and purified by column chromatography to afford the desired product 6-cyano-1-methyl-4-(4,4,5,5-tetramethyl-1,3,2-dioxaborolan-2-yl)-1H-indazole as a yellow solid (0.9 g, 75%). ¹H NMR (300 MHz, CDCl₃): δ 8.40 (s, 1H), 7.84 (s, 2H), 4.12 (s, 3H), 1.40 (s, 12H). ESI-MS (m/z): 284.0 (M+H)⁺.

### 2-Chloro-4-(6-cyano-1-methyl-1H-indazol-4-yl)pyrimidine

A mixture of 6-cyano-1-methyl-4-(4,4,5,5-tetramethyl-1,3,2-dioxaborolan-2-yl)-1H-indazole (1.1 g, 4.2 mmol, 1.0 eq), 2,4-dichloropyrimidine (798 mg, 4.2 mmol, 1.0 eq), Pd(dppf)Cl₂ (154 mg, 0.21 mmol, 0.05 eq) and K₂CO₃ (1.73 g, 12.6 mmol, 3.0 eq) in dioxane (25 mL) and H₂O (5 mL) was heated to 80° C. and stirred for 2 h under N₂. LCMS showed no starting materials left, the reaction mixture was filtered through celite, concentrated in vacuo to afford a crude product, which was further purified by column chromatography to afford the desired product 2-chloro-4-(6-cyano-1-methyl-1H-indazol-4-yl)pyrimidine as a yellow solid (0.7 g, 62%). ¹HNMR (300 MHz, DMSO-d₆): δ 8.95 (br s, 1H), 8.76 (s, 1H), 8.68 (s, 1H), 8.40-8.36 (m, 2H), 4.20 (s, 3H). ESI-MS (m/z): 270.0 (M+H)⁺.

### A10. 2-Chloro-4-(1-methyl-1H-indazol-4-yl)pyrimidine

### 4-Bromo-1-methyl-1H-indazole

Methylhydrazine (7.56 g, 69.6 mmol) was added to a solution of 2-bromo-6-fluorobenzaldehyde (2.0 g, 9.95 mmol, 1.0 eq) in DMSO (35 mL). The mixture was heated to 85° C. and stirred for 24 hours. It was then cooled to room temperature and diluted with water (50 mL). The solution was extracted with CH₂Cl₂ (2×50 mL) and the combined organic layers were dried (Mg₂SO₄), filtered, and concentrated under reduced pressure to give a crude residue of 4-bromo-1-methyl-1H-indazole (1.5 g, crude), which was used without further purification. ¹H NMR (300 MHz, CDCl₃): δ 8.01 (s, 1H), 7.35-7.28 (m, 3H), 4.10 (s, 3H). ESI-MS (m/z): 211.0 (M+H)⁺.

### 1-Methyl-4-(4,4,5,5-tetramethyl-1,3,2-dioxaborolan-2-yl)-1H-indazole

A 100 mL three-necked flask was charged with 4-bromo-1-methyl-1H-indazole (1.38 g, 6.57 mmol, 1.0 eq), bis(pinacolato)diboron (2.34 g, 8.54 mmol, 1.3 eq), KOAc (2.09 g, 19.71 mmol, 3.0 eq) and PdCl₂(dppf) CH₂Cl₂ complex (0.29 g, 0.32 mmol, 0.05 eq) under argon. Dry DMSO (22 mL) was added and the mixture was heated at 90° C. for 4 h. The reaction mixture was cooled, filtered and the filter cake was washed with TBME (2×50 mL). The filtrate was washed with brine (3×50 mL), dried over Na₂SO₄, concentrated and purified by silica column to give the desired product 1-methyl-4-(4,4,5,5-tetramethyl-1,3,2-dioxaborolan-2-yl)-1H-indazole (1.0 g, 60%). ¹H NMR (300 MHz, CDCl₃): δ 8.37 (s, 1H), 7.67-7.66 (m, 1H), 7.51-7.50 (m, 1H), 7.42-7.40 (m, 1H), 4.10 (s, 3H), 1.42 (s, 12H). ESI-MS (m/z): 259.1 (M+H)⁺.

### 2-Chloro-4-(1-methyl-1H-indazol-4-yl)pyrimidine

To a solution of 1-methyl-4-(4,4,5,5-tetramethyl-1,3,2-dioxaborolan-2-yl)-1H-indazole (774 mg, 3.0 mmol, 1.0 eq) in 1,4-dioxane/water (5/1, 10 mL) was added 2,4-dichloropyrimidine (542 mg, 3.60 mmol, 1.2 eq), potassium carbonate (954 mg, 9.0 mmol, 3.0 eq), and (dppf)₂PdCl₂ (108 mg, 0.15 mmol, 0.05 eq) under argon. The mixture was purged with argon at room temperature for 10 min and refilled with argon, heated to reflux and stirred for 4 h, when TLC indicated completion. The reaction mixture was concentrated to give a crude residue, which was purified by silica column to give the desired product 2-chloro-4-(1-methyl-1H-indazol-4-yl)pyrimidine as a yellow solid (300 mg, 40%). ¹H NMR (300 MHz, CDCl₃): δ 8.73 (sl br s, 2H), 7.83-7.75 (m, 2H), 7.62-7.55 (m, 2H), 4.19 (s, 3H). ESI-MS (m/z): 245.0 (M+H)⁺.

### A11. 2-Chloro-4-(1,3-dimethyl-1H-pyrrolo[2,3-b]pyridin-5-yl)pyrimidine

### 5-Bromo-1,3-dimethyl-1H-pyrrolo[2,3-b]pyridine

To a solution of 5-bromo-3-methyl-1H-pyrrolo[2,3-b]pyridine (1.0 g, 4.74 mmol, 1 eq) in DMF (20 mL) at 0° C. was added NaH (0.21 g, 5.2 mmol, 1.1 eq) portion wise, and the mixture was stirred at this temperature for 30 minutes. Then MeI (0.74 g, 5.2 mmol, 1.1 eq) was added dropwise. After addition, the mixture was stirred at this temperature for 30 minutes till completion. The mixture was poured into water (70 mL), and extracted with EA (50 mL×3). The combined organic layers were washed with brine twice, dried over sodium sulfate, filtered and the filtrate was concentrated in vacuo to give a residue, which was purified by silica gel column chromatography (EtOAc/Hexane, 1/10) to afford 5-bromo-1,3-dimethyl-1H-pyrrolo[2,3-b]pyridine (0.8 g, 75%). ¹H NMR (300 MHz, CDCl₃): δ 8.33 (s, 1H), 7.96 (s, 1H), 6.96 (s, 1H), 3.82 (s, 3H), 2.28 (s, 3H). ESI-MS (m/z): 225.0 (M+H)⁺.

### 1,3-Dimethyl-5-(4,4,5,5-tetramethyl-1,3,2-dioxaborolan-2-yl)-H-pyrrolo[2,3-b]pyridine

To a solution of 5-bromo-1,3-dimethyl-1H-pyrrolo[2,3-b]pyridine (0.8 g, 3.55 mmol, 1.0 eq) in 1,4-dioxane (8 mL) were added bis(pinacolato)diboron(1.17 g, 4.62 mmol, 1.3 eq), KOAc (1.045 g, 10.66 mmol, 3.0 eq) and Pd(dppf)Cl₂DCM (290 mg, 0.355 mmol, 0.1 eq) under nitrogen atmosphere. The mixture was purged with nitrogen 3 times, and stirred at 90° C. for 2 hours. After cooling to room temperature, the mixture was concentrated and the residue was purified by chromatography on silica gel to give 1,3-dimethyl-5-(4,4,5,5-tetramethyl-1,3,2-dioxaborolan-2-yl)-1H-pyrrolo[2,3-b]pyridine (0.79 g, 82%). ¹H NMR (300 MHz, CDCl₃): δ 8.71 (s, 1H), 8.33 (s, 1H), 6.93 (s, 1H), 3.86 (s, 3H), 2.32 (s, 3H), 1.39 (s, 12H). ESI-MS (m/z): 273.1 (M+H)⁺.

### 2-Chloro-4-(1,3-dimethyl-1H-pyrrolo[2,3-b]pyridin-5-yl)pyrimidine

To a solution of 1,3-dimethyl-5-(4,4,5,5-tetramethyl-1,3,2-dioxaborolan-2-yl)-1H-pyrrolo[2,3-b]pyridine (0.8 g, 2.94 mmol, 1.0 eq) in 1,4-dioxane (20 mL) and water (4 mL) were added 2,4-dichloropyrimidine (0.482 g, 3.23 mmol, 1.1 eq), K₂CO₃ (1.217 g, 8.82 mmol, 3.0 eq) and Pd(dppf)Cl₂DCM (240 mg, 0.294 mmol, 0.1 eq) under nitrogen. The mixture was bubbled with nitrogen for 10 minutes, then purged with nitrogen 3 times and stirred at 60° C. for 2.5 hours. After cooling, the mixture was concentrated and the residue was purified by chromatography on silica gel to give 2-chloro-4-(1,3-dimethyl-1H-pyrrolo[2,3-b]pyridin-5-yl)pyrimidine (0.53 g, 70%). ¹H NMR (300 MHz, CDCl₃): δ 8.98 (s, 1H), 8.72-8.57 (m, 2H), 7.73 (sl br s, 1H), 7.04 (s, 1H), 3.90 (s, 3H), 2.40 (s, 3H). ESI-MS (m/z): 259.1 (M+H)⁺.

### A12. 2-Chloro-4-(1,3-dimethyl-1H-indol-5-yl)pyrimidine

### 5-Bromo-3-methyl-1H-indole

To a solution of 5-bromo-1H-indole-3-carbaldehyde (5.0 g, 22.4 mmol, 1.0 eq) in THF (100 mL) was added LiAlH₄ (1.02 g, 26.9 mmol, 1.2 eq). The resulting solution was stirred for 2 h under reflux, then poured into 1 N NaOH solution (300 mL), filtered and the filter cake was washed with EA, the aqueous layer was separated and was extracted with ethyl acetate (3×150 mL), dried over anhydrous sodium sulfate and then concentrated under vacuum to give a residue, which was purified via silica gel chromatography (3% ethyl acetate in petroleum ether) to afford 5-bromo-3-methyl-1H-indole (2.5 g, 53%). ¹H NMR (300 MHz, CDCl₃): δ 7.97 (br, 1H), 7.73 (s, 1H), 7.28-7.26 (m, 2H), 7.00 (s, 1H), 2.31 (s, 3H).

### 5-Bromo-1,3-dimethyl-1H-indole

A solution of 5-bromo-3-methyl-1H-indole (5.0 g, 23.8 mmol, 1.0 eq) in DMF (100 mL) under nitrogen was cooled down to 0° C., NaH (1.05 g, 26.2 mmol, 1.1 eq) was added carefully and the mixture was stirred at this temperature for 30 minutes. Then MeI (3.72 g, 26.2 mmol, 1.1 eq) was added drop wise. After addition, the mixture was stirred at this temperature for 30 minutes till completion. The reaction was quenched with water (300 mL), extracted with EA (150 mL×3). The combined organic layers were washed with brine twice, dried over sodium sulfate, filtered and the filtrate was concentrated in vacuo to give a residue which was purified by silica gel column chromatography (EtOAc/Hexane, 1/10) to afford 5-bromo-1,3-dimethyl-1H-indole (5.11 g, 95%). ¹H NMR (300 MHz, CDCl₃): δ 7.71 (s, 1H), 7.30-7.29 (m, 1H), 7.17-7.15 (m, 1H), 6.84 (s, 1H), 3.73 (s, 3H), 2.36 (s, 3H).

### 1,3-Dimethyl-5-(4,4,5,5-tetramethyl-1,3,2-dioxaborolan-2-yl)-1H-indole

To a solution of 5-bromo-1,3-dimethyl-1H-indole (0.54 g, 2.4 mmol, 1.0 eq) in 1,4-dioxane (10 mL) were added bis(pinacolato)diboron (0.796 g, 3.13 mmol, 1.3 eq), KOAc (0.708 g, 7.23 mmol, 3.0 eq) and Pd(dppf)Cl₂DCM (196 mg, 0.24 mmol, 0.1 eq) under nitrogen. The mixture was purged with nitrogen 3 times and stirred at 90° C. for 2 hours. After cooling the mixture was concentrated and the residue was purified by chromatography on silica gel to give 1,3-dimethyl-5-(4,4,5,5-tetramethyl-1,3,2-dioxaborolan-2-yl)-1H-indole (520 mg, 79%). ¹H NMR (300 MHz, CDCl₃): δ 8.12 (s, 1H), 7.69-7.66 (m, 1H), 7.28-7.26 (m, 1H), 6.82 (s, 1H), 3.75 (s, 3H), 2.36 (s, 3H), 1.40 (s, 12H).

### 2-Chloro-4-(1,3-dimethyl-1H-indol-5-yl)pyrimidine

To a solution of 1,3-dimethyl-5-(4,4,5,5-tetramethyl-1,3,2-dioxaborolan-2-yl)-1H-indole (0.52 g, 1.92 mmol, 1.0 eq) in 1,4-dioxane (13 mL) and water (2.6 mL) were added 2,4-dichloropyrimidine (0.314 g, 2.11 mmol, 1.1 eq), K₂CO₃ (0.794 g, 5.75 mmol, 3.0 eq) and Pd(dppf)Cl₂DCM (0.156 g, 0.192 mmol, 0.1 eq) under nitrogen. The mixture was bubbled with nitrogen for 10 minutes, then purged with nitrogen 3 times and stirred at 60° C. for 2.5 hours. After cooling the mixture was concentrated and the residue was purified by chromatography on silica gel to give 2-chloro-4-(1,3-dimethyl-1H-indol-5-yl)pyrimidine (0.3 g, 60%). ¹H NMR (300 MHz, CDCl₃): δ 8.57 (d, J=4.8 Hz, 1H), 8.40 (s, 1H), 7.99 (d, J=8.1 Hz, 1H), 7.74 (d, J=4.2 Hz, 1H), 7.36 (d, J=8.7 Hz, 1H), 6.91 (s, 1H), 3.80 (s, 3H), 2.40 (s, 3H).

### A13. 2-Chloro-4-(3-chloro-1-methyl-1H-pyrrolo[2,3-b]pyridin-5-yl)pyrimidine

### 5-Bromo-3-chloro-1H-pyrrolo[2,3-b]pyridine

To a solution of 5-bromo-1H-pyrrolo[2,3-b]pyridine (5.0 g, 25.4 mmol, 1.0 eq) in THF (100 mL) was added N-chlorosuccinimide (4.0 g, 30.4 mmol, 1.2 eq) and the mixture was stirred at room temperature for 24 h. Water (100 mL) was added to the reaction mixture, followed by extraction with EA (3×80 mL). The combined organic layer was dried over Mg₂SO₄, filtered and the filtrate was concentrated in vacuo to give a crude residue, which was purified by silica gel column chromatography (EtOAc/Hexane, 1/5) to afford 5-bromo-3-chloro-1H-pyrrolo[2,3-b]pyridine (5.0 g, 85%). ¹H NMR (300 MHz, DMSO-d₆): δ 12.26 (br, 1H), 8.37 (s, 1H), 8.16 (s, 1H), 7.79 (s, 1H). ESI-MS (m/z): 232.9 (M+H)⁺.

### 5-Bromo-3-chloro-1-methyl-1H-pyrrolo[2,3-b]pyridine

To a cooled solution of 5-bromo-3-chloro-1H-pyrrolo[2,3-b]pyridine (5.0 g, 21.6 mmol, 1.0 eq) in DMF (100 mL) under nitrogen was added NaH (1.0 g, 26.0 mmol, 1.2 eq) carefully and the mixture was stirred at 0° C. for 30 minutes. Then MeI (3.7 g, 26.0 mmol, 1.2 eq) was added drop wise. After addition, the mixture was stirred at this temperature for 30 minutes till completion. The mixture was poured into water (200 mL), extracted with EA (150 mL×3). The combined organic layers were washed with brine twice, dried over sodium sulfate, filtered and the filtrate was concentrated in vacuo to give a residue, which was purified by silica gel column chromatography (EtOAc/Hexane, 1/10) to afford 5-bromo-3-chloro-1-methyl-1H-pyrrolo[2,3-b]pyridine (3.8 g, 71%). ¹H NMR (300 MHz, CDCl₃): δ 8.40 (s, 1H), 8.05 (s, 1H), 7.19 (s, 1H), 3.87 (s, 3H). ESI-MS (m/z): 246.9 (M+H)⁺.

### 3-Chloro-1-methyl-5-(4,4,5,5-tetramethyl-1,3,2-dioxaborolan-2-yl)-1H-pyrrolo[2,3-b]pyridine

To a solution of 5-bromo-3-chloro-1-methyl-1H-pyrrolo[2,3-b]pyridine (0.2 g, 0.82 mmol, 1.0 eq) in 1,4-dioxane (2 mL) were added bis(pinacolato)diboron (0.269 g, 1.06 mmol, 1.3 eq), KOAc (0.240 g, 2.45 mmol, 3 eq) and Pd(dppf)Cl₂DCM (66 mg, 0.08 mmol, 0.1 eq) under nitrogen. The mixture was purged with nitrogen 3 times and stirred at 90° C. for 2 hours. After cooling the mixture was concentrated and the residue was purified by chromatography on silica gel to give 3-chloro-1-methyl-5-(4,4,5,5-tetramethyl-1,3,2-dioxaborolan-2-yl)-1H-pyrrolo[2,3-b]pyridine (0.2 g, 84%). ¹H NMR (300 MHz, CDCl₃): δ 8.74 (s, 1H), 8.39 (s, 1H), 7.15 (s, 1H), 3.89 (s, 3H), 1.40 (s, 12H). ESI-MS (m/z): 293.1 (M+H)⁺.

### 2-Chloro-4-(3-chloro-1-methyl-1H-pyrrolo[2,3-b]pyridin-5-yl)pyrimidine

To a solution of 3-chloro-1-methyl-5-(4,4,5,5-tetramethyl-1,3,2-dioxaborolan-2-yl)-1H-pyrrolo[2,3-b]pyridine (0.2 g, 0.685 mmol, 1.0 eq) in 1,4-dioxane (5 mL) and water (1 mL) were added 2,4-dichloropyrimidine (0.112 g, 0.753 mmol, 1.1 eq), K₂CO₃ (0.284 g, 2.05 mmol, 3.0 eq) and Pd(dppf)Cl₂DCM (0.056 g, 0.068 mmol, 0.1 eq) under nitrogen. The mixture was bubbled with nitrogen for 10 minutes, then purged with nitrogen 3 times and stirred at 60° C. for 2.5 hours. After cooling the mixture was concentrated and the residue was purified by chromatography on silica gel to give 2-chloro-4-(3-chloro-1-methyl-1H-pyrrolo[2,3-b]pyridin-5-yl)pyrimidine (0.1 g, 62%). ¹H NMR (300 MHz, DMSO-d₆): δ 9.17 (s, 1H), 8.85-8.75 (m, 1H), 8.71 (s, 1H), 8.37-8.29 (m, 1H), 7.90 (s, 1H), 3.87 (s, 3H). ESI-MS (m/z): 279.0 (M+H)⁺.

### A14. 2-(1-(2-Chloropyrimidin-4-yl)-1H-indol-3-yl)-2-oxoethyl Acetate

### 2-(1H-indol-3-yl)-2-oxoethyl acetate

To a solution of 1H-indole (11.7 g, 100 mmol, 1.0 eq) in toluene (200 ml) was added pyridine (7.9 g, 100 mmol, 1.0 eq). The mixture was heated to 60° C. and then 2-chloro-2-oxoethyl acetate (13.6 g, 100 mmol, 1.0 eq) was slowly added dropwise. After addition, the mixture was stirred for 1 hour at 60° C., cooled to room temperature and mixed with MeOH/H₂O (200 mL) and stirred for 1 hour. After completion, the mixture was filtered, and purified by silica column chromatography affording the desired product 2-(1H-indol-3-yl)-2-oxoethyl acetate (1 g, 5%). ¹H NMR (300 MHz, DMSO-d₆): δ 8.80 (br, 1H), 8.35-8.34 (m, 1H), 7.91 (s, 1H), 7.44-7.43 (m, 1H), 7.33-7.27 (m, 2H), 5.22 (s, 2H), 2.21 (s, 3H). ESI-MS (m/z): 217.9 (M+H)⁺.

### 2-(1-(2-Chloropyrimidin-4-yl)-1H-indol-3-yl)-2-oxoethyl acetate

To a solution of 2-(1H-indol-3-yl)-2-oxoethyl acetate (1.08 g, 5.0 mmol, 1.0 eq) in DMF (10 mL) was added 60% NaH (300 mg, 7.5 mmol, 1.5 eq) at 0° C. over a period of 20 min. After addition, the reaction was stirred at 0° C. for 20 minutes, then 2,4-dichloropyrimidine (820 mg, 5.5 mmol, 1.1 eq) in DMF (2 mL) was added at 0° C. The reaction mixture was stirred for 1 hour at room temperature, quenched with H₂O (10 mL), and extracted with EA (20 mL). The organic layer was washed with brine, dried over Na₂SO₄, concentrated in vacuo and purified by silica column chromatography affording the desired product 2-(1-(2-chloropyrimidin-4-yl)-1H-indol-3-yl)-2-oxoethyl acetate (280 mg, 18%). ¹H NMR (300 MHz, CDCl₃): δ 8.97 (s, 1H), 8.78 (d, J=7.5 Hz, 1H), 8.67 (d, J=5.1 Hz, 1H), 8.42 (d, J=7.8 Hz, 1H), 7.54-7.44 (m, 2H), 7.26-7.25 (m, 1H), 5.31 (s, 2H), 2.28 (s, 3H). ESI-MS (m/z): 329.9 (M+H)⁺.

### A15. N-(1-(2-Chloropyrimidin-4-yl)-1H-indazol-3-yl)methanesulfonamide

To a solution of 3-amino-1-(2-chloropyrimid-4-yl)indazole (500 mg, 2.0 mmol, 1.0 eq) in DCM (10 mL) was added DIPEA (310 mg, 2.4 mmol, 1.2 eq) and MsCl (275 mg, 2.4 mmol, 1.2 eq) at 0° C. The mixture was stirred at RT for 2 h, TLC indicated completion. The reaction was diluted with DCM (30 mL), washed with brine (30 mL×3), dried over sodium sulfate, concentrated and purified on silica column affording N-(1-(2-chloropyrimidin-4-yl)-1H-indazol-3-yl)methanesulfonamide (290 mg, 44%). ¹H NMR (300 MHz, DMSO-d₆): δ 11.55-11.30 (br, 1H), 8.69 (d, J=5.7 Hz, 1H), 8.58 (t, J=8.2 Hz, 1H), 8.06 (dd, J=8.411 Hz, 1H), 7.82-7.66 (m, 2H), 7.46-7.38 (m, 1H). 3.48 (d, J=6.3 Hz, 3H). ESI-MS (m/z): 321.7 (M−H)⁻.

### A16. 2-Chloro-4-(1-(N-methylamino)imidazo[1,5-a]pyridin-3-yl)pyrimidine

### 2-Chloro-N-(pyridin-2-ylmethyl)pyrimidine-4-carboxamide

To a solution of 2-chloropyrimidine-4-carboxylic acid (14.0 g, 88.3 mmol, 1.0 eq) in DCM (100 mL) were added pyridin-2-ylmethanamine (11.4 g, 106 mmol, 1.2 eq), DIPEA (28.5 g, 221 mmol, 2.5 eq) and HATU (50.4 g, 132.5 mmol, 1.5 eq), the mixture was stirred at 25° C. overnight. TLC and LCMS indicated completion. The mixture was cooled to 0° C., quenched with water (100 mL), and extracted with DCM (50 mL×3). The combined organic layers were dried over sodium sulfate, concentrated and purified by silica column affording 2-chloro-N-(pyridin-2-ylmethyl)pyrimidine-4-carboxamide (5 g, 23%). ¹H NMR (300 MHz, DMSO-d₆): δ 9.56 (br, 1H), 9.03 (d, J=4.8 Hz, 1H), 8.52 (d, J=3.9 Hz, 1H), 8.05 (d, J=4.5 Hz, 1H), 7.78-7.73 (m, 1H), 7.34-7.25 (m, 2H), 4.61 (d, J=5.7 Hz, 2H). ESI-MS (m/z): 248.9 (M+H)⁺.

### 2-Chloro-4-(imidazo[1,5-a]pyridin-3-yl)pyrimidine

A solution of 2-chloro-N-(pyridin-2-ylmethyl)pyrimidine-4-carboxamide (5.0 g, 20.2 mmol, 1.0 eq) in POCl₃ (75 mL) was refluxed for 7 hours. After TLC and LCMS indicated completion, the mixture was cooled to RT and poured into ice-water, extracted with EA (100 mL×3). The combined organic layers were washed with brine (100 mL×2), dried over sodium sulfate, and concentrated under reduced pressure affording 2-chloro-4-(imidazo[1,5-a]pyridin-3-yl)pyrimidine (4 g, 87%). ¹H NMR (300 MHz, DMSO-d₆): δ 9.67 (d, J=6.3 Hz, 1H), 8.69 (d, J=4.8 Hz, 1H), 8.11 (d, J=5.1 Hz, 1H), 7.91-7.84 (m, 2H), 7.25-7.16 (m, 2H). ESI-MS (m/z): 230.9 (M+H)⁺.

### 2-Chloro-4-(1-nitroimidazo[1,5-a]pyridin-3-yl)pyrimidine

To a solution of 2-chloro-4-(imidazo[1,5-a]pyridin-3-yl)pyrimidine (5.0 g, 21.6 mmol, 1.0 eq) in AcOH (100 mL) was added a mixture of HOAc and HNO₃ (1/1, 50 mL) dropwise at 10-15° C. The mixture was stirred at RT for 30 mins. After TLC and LCMS indicated completion, the mixture was diluted with water (50 mL), the precipitate formed was collected by filtration, washed with water, and dried, giving the desired product 2-chloro-4-(1-nitroimidazo[1,5-a]pyridin-3-yl)pyrimidine which was used in next step directly. ¹H NMR (300 MHz, DMSO-d₆): δ 9.92 (d, J=6.9 Hz, 1H), 9.14 (d, J=4.8 Hz, 1H), 8.47 (d, J=9.0 Hz, 1H), 8.26-8.18 (m, 1H), 7.92-7.88 (m, 1H), 7.65-7.63 (m, 1H). ESI-MS (m/z): 275.8 (M+H)⁺.

### 2-Chloro-4-(1-aminoimidazo[1,5-a]pyridin-3-yl)pyrimidine

To a solution of 2-chloro-4-(1-nitroimidazo[1,5-a]pyridin-3-yl)pyrimidine (5.0 g, 18.1 mmol, 1.0 eq) in EtOH and water (10/1, 100 mL) was added NH₄Cl (2.9 g, 54.3 mmol, 3.0 eq) and Fe powder (10.0 g, 181 mmol, 10.0 eq). The mixture was stirred at RT for 5 h. After TLC and LCMS indicated completion, the mixture was filtered. The filtrate was concentrated and purified by silica column affording desired product 2-chloro-4-(1-aminoimidazo[1,5-a]pyridin-3-yl)pyrimidine (600 mg, 13.6%). ESI-MS (m/z): 245.9 (M+H)⁺.

### 2-Chloro-4-(1-(2,2,2-trifluoroacetamido)imidazo[1,5-a]pyridin-3-yl)pyrimidine

To a solution of 2-chloro-4-(1-aminoimidazo[1,5-a]pyridin-3-yl)pyrimidine (245 mg, 1.0 mmol, 1.0 eq) in DCM (4 mL) was added TEA (303 mg, 3.0 mmol, 3.0 eq), DMAP (22 mg, 0.1 mmol, 0.1 eq) and trifluoroacetic anhydride (252 mg, 1.2 mmol, 1.2 eq) at 0° C. The mixture was stirred at RT for 3 h. After TLC and LCMS indicated completion, the mixture was concentrated and purified on silica column 2-chloro-4-(1-(2,2,2-trifluoroacetamino)imidazo[1,5-a]pyridin-3-yl)pyrimidine (300 mg, 87%). ¹H NMR (300 MHz, DMSO-d₆): δ 12.02 (s, 1H), 9.72 (d, J=5.1 Hz, 1H), 8.73 (d, J=5.4 Hz, 1H), 8.05 (d, J=5.1 Hz, 1H), 7.76 (d, J=6 Hz, 1H), 7.29 (d, J=5.4 Hz, 2H). LCMS: (M+H)⁺:341.8.

### 2-Chloro-4-(1-(N-methyl-2,2,2-trifluoroacetamido)imidazo[1,5-a]pyridin-3-yl)pyrimidine

To a solution of 2-chloro-4-(1-(2,2,2-trifluoroacetamido)imidazo[1,5-a]pyridin-3-yl)pyrimidine (459 mg, 1.3 mmol, 1.0 eq) in DMF (5 mL) was added 60% NaH (65 mg, 1.6 mmol, 1.2 eq) portion wise at 0° C., and the mixture was kept at 0° C. for 30 minutes. Then MeI (203 mg, 1.4 mmol, 1.1 eq) was added dropwise. After addition, the mixture was stirred at room temperature for 2 h. The reaction mixture was poured into water (50 mL), extracted with DCM (50 mL×3), the combined organic layers were washed with brine (50 mL×3), dried over sodium sulfate, concentrated to give the desired product 2-chloro-4-(1-(N-methyl-2,2,2-trifluoroacetamido)imidazo[1,5-a]pyridin-3-yl)pyrimidine (500 mg, crude) which was used in next step directly without purification. ESI-MS (m/z): 355.8 (M+H)⁻.

### 2-Chloro-4-(1-(N-methylamino)imidazo[1,5-a]pyridin-3-yl)pyrimidine

To a solution of 2-chloro-4-(1-(N-methyl-2,2,2-trifluoroacetamino)imidazo[1,5-a]pyridin-3-yl)pyrimidine (500 mg, 0.95 mmol, 1.0 eq) in MeOH (10 mL) was added K₂CO₃ (131 mg, 0.95 mmol, 1.0 eq). After stirring for 30 mins, TLC indicated completion. The mixture was concentrated and diluted with water (50 mL), extracted with DCM (50 mL×2). The organic layer was washed with brine (50 mL×2), dried over sodium sulfate, purified on silica column affording 2-chloro-4-(1-(N-methylamino)imidazo[1,5-a]pyridin-3-yl)pyrimidine (148 mg, 40%). ¹H NMR (300 MHz, DMSO-d₆): δ 9.54 (d, J=7.2 Hz, 1H), 8.42 (d, J=5.4 Hz, 1H), 7.84 (d, J=8.1 Hz, 2H), & 0.77 (d, J=5.7 Hz, 1H), 7.12 (t, J=6.9 Hz, 1H), 6.97-6.93 (m, 1H), 6.40-6.38 (m, 1H), 2.97 (d, J=4.5 Hz, 3H). ESI-MS (m/z): 259.9 (M+H)⁺.

### A17. Methyl 1-(2-chloropyrimidin-4-yl)-1H-indole-3-carboxylate

To a 500 mL four-neck flask were added THF (300 mL) and methyl indole-3-carboxylate (28.0 g, 159 mmol, 1.0 eq). The solution was cooled to 0° C. and t-BuOK (21.5 g, 191 mmol, 1.2 eq) was added portion wise. After stirring at this temperature for 1 h, 2,4-dichloropyrimidine (23.8 g, 159 mmol, 1.0 eq) was added and the mixture was warmed to RT and stirred for 2 h till completion. The reaction was quenched with saturated NH₄Cl (34 mL), diluted with water (500 mL) and then filtered and the filtrate was extracted with DCM (100 mL×3). The combined organic layer was dried over Na₂SO₄, concentrated and purified by column chromatography on silica to give the product (7 g, 16%) as brown solid. ¹H NMR (300 MHz, CDCl₃) δ 8.70-8.68 (m, 1H), 8.55-8.45 (m, 1H), 8.36-8.26 (m, 1H), 7.57-7.32 (m, 4H), 3.99 (s, 3H).

### A18. 2-Chloro-4-(3-(N,N-dimethylamino)-1H-pyrazolo[4,3-b]pyridin-1-yl) pyrimidine

### 3-Amino-1H-pyrazolo[4,3-b]pyridine

A mixture of 2-cyano-3-fluoropyridine (40 g, 328 mmol, 1.0 eq) and hydrazine hydrate (47.8 mL, 984 mmol, 3.0 eq) in n-butanol (400 mL) was heated to reflux under nitrogen overnight. The reaction mixture was allowed to cool to room temperature, water (300 mL) was added, the phases were separated, and the organic phase was concentrated under reduced pressure. The residual solid was collected by filtration and washed with water, dried to give 3-amino-1H-pyrazolo[4,3-b]pyridine as a yellow solid (31 g, 73%). ¹H NMR (300 MHz, CDCl₃): δ 11.64 (s, 1H), 8.27 (sl br s, 1H), 7.69 (d, J=8.4 Hz, 1H), 7.25-7.22 (m, 1H), 5.37 (br, 2H). ESI-MS (m/z): 135.0 (M+H)⁺.

### 2-Chloro-4-(3-amino-1H-pyrazolo[4,3-b]pyridin-1-yl)pyrimidine

To a solution of 3-amino-1H-pyrazolo[4,3-b]pyridine (6.2 g, 1.0 eq) in DMF (95 mL) at 0° C. was added t-BuOK (6.2 g, 1.2 eq) portion wise, after addition, the mixture was stirred at 0° C. for 30 min. A solution of 2,4-dichloropyrimidine (7.5 g, 1.1 eq) in DMF (50 mL) was added drop wise at 0° C. After the addition completed, the reaction mixture was stirred at rt for 4 h, LCMS indicated starting material disappeared, H₂O (400 mL) was added, the precipitates were filtered and dried to afford the desired product 2-chloro-4-(3-amino-1H-pyrazolo[4,3-b]pyridin-1-yl)pyrimidine (5.5 g, 48%) as a yellow solid. ¹H NMR (300 MHz, CDCl₃): δ 8.76 (d, J=7.8 Hz, 1H), 8.62-8.57 (m, 2H), 7.67-7.55 (m, 2H), 6.76 (br, 2H). ESI-MS (m/z): 246.9 (M+H)⁺.

### 2-Chloro-4-(3-(N,N-dimethylamino)-1H-pyrazolo[4,3-b]pyridin-1-yl)pyrimidine

To a solution of 2-chloro-4-(3-amino-1H-pyrazolo[4,3-b]pyridin-1-yl)pyrimidine (5.5 g, 22.3 mmol, 1.0 eq) in dry DMF (110 mL) at 0° C. was added NaH (1.78 g, 44.7 mmol, 60% dispersion in mineral oil, 2.2 eq) portion wise. After stirred for 30 minutes at 0° C., methyl iodide (6.9 g, 48.5 mmol, 2.2 eq) was added drop wise, afterwards the reaction mixture was allowed to warm to room temperature, LC-MS indicated starting material disappeared. H₂O (300 mL) was carefully added and the aqueous phase was extracted using EtOAc. The combined organic layers were washed with H₂O and sat. NaCl. Then the combined organic layers were dried over Na₂SO₄ and after filtration the solvent was removed in vacuo to afford the crude product, which was further purified by column chromatography to afford 2-chloro-4-(3-(N,N-dimethylamino)-1H-pyrazolo[4,3-b]pyridin-1-yl) pyrimidine (500 mg, 8%). ¹HNMR (300 MHz, CDCl₃): δ 9.02 (d, J=9.0 Hz, 1H), 8.61 (d, J=3.0 Hz, 1H), 8.46 (d, J=6.3 Hz, 1H), 7.70 (d, J=5.7 Hz, 1H), 7.50-7.45 (m, 1H), 3.45 (s, 6H). ESI-MS (m/z): 274.9 (M+H)⁺.

### A19. 2-Chloro-4-(3-(methylamino)-1H-thieno[2,3-c]pyrazol-1-yl)pyrimidine

### 2-Bromo-N-methyl-N′-tosylthiophene-3-carbohamidrazone

A mixture of N′-(2-bromo thiophene-3-carbonyl)-4-methylbenzenesulfonohydrazide (10 g, 26.7 mmol) in thionyl chloride (18.9 g, 160 mmol) was heated to 80° C. for 1 hour. The reaction mixture was allowed to cool to room temperature and concentrated in vacuo to give a crude residue. The residue was dissolved in THF (150 mL) at 0° C. and DABCO (5.98 g, 53.4 mmol) was added, then methylamine solution in THF (53.4 mL) was added dropwise. The reaction was warmed to room temperature and stirred at overnight. The reaction was concentrated in vacuo to remove the solvent and water (200 mL) was added, extracted with DCM (150 mL×3), the combined organic layers were dried over anhydrous sodium sulfate and concentrated in vacuo to give a crude residue, which was purified on silica gel chromatography to give 2-bromo-N-methyl-N′-tosylthiophene-3-carbamidrazone as yellow solid (2 g, 18%). ¹H NMR (300 MHz, DMSO-d₆): δ7.87-7.84 (m, 2H), 7.30-7.28 (m, 3H), 6.84 (d, J=5.7 Hz, 1H), 2.67 (s, 3H), 2.40 (s, 3H). ESI-MS (m/z): 388.0 (M+H)⁺.

### 3-(Methylamino)-1-tosyl-1H-thieno[2,3-c]pyrazole

A mixture of 2-bromo-N-methyl-N′-tosylthiophene-3-carbamidrazone (970 mg, 2.5 mmol), CuI (95 mg, 0.5 mmol), and K₂CO₃ (690 mg, 5 mmol) in NMP (10 mL) was heated to 110° C. in a microwave for 20 min. LC-MS indicated starting materials disappeared. The reaction mixture was poured into 10 mL water and filtered, the filter cake was washed with water, and dried to give 3-(methylamino)-1-tosyl-1H-thieno[2,3-c]pyrazole (400 mg, 52%). ¹H NMR (400 MHz, DMSO-d₆): δ7.65 (d, J=8.0 Hz, 2H), 7.35 (d, J=8.0 Hz, 2H), 7.28 (d, J=5.6 Hz, 1H), 6.93 (d, J=5.6 Hz, 1H), 6.84 (br, 1H), 2.72 (s, 3H), 2.32 (s, 3H). ESI-MS (m/z): 308.0 (M+H)³⁰.

### 3-(Methylamino)-1H-thieno[2,3-c]pyrazole

To a solution of 3-methylamino-1-tosyl-1H-thieno[2,3-c]pyrazole (1.23 g, 4 mmol) in methanol (40 mL) was added magnesium powder (480 mg, 20 mmol). The mixture was stirred at rt for 30 minutes. The solvent was removed under reduced pressure. The resulting residue was taken up in DCM (40 mL) and washed with water (25 mL), dried over sodium sulfate and concentrated under reduced pressure. The residue was purified by silica gel chromatography to give 3-(methylamino)-1H-thieno[2,3-c]pyrazole (180 mg, 27%). ¹HNMR (300 MHz, CDCl₃): δ 6.90-7.75 (br, 2H), 6.85 (d, J=5.4 Hz, 1H), 6.72 (d, J=5.4 Hz, 1H), 3.04 (s, 3H). ESI-MS (m/z): 154.1 (M+H)⁺.

### 2-Chloro-4-(3-(methylamino)-1H-thieno[2,3-c]pyrazol-1-yl)pyrimidine

To a solution of 3-(methylamino)-1H-thieno[2,3-c]pyrazole (168 mg, 1.08 mmol) in THF (2 mL) was added ^{t}BuOK (181 mg, 1.62 mmol) at 0° C. The mixture was stirred at this temperature for 30 minutes, then a solution of 2,4-dichloropyrimidine (192 mg, 1.3 mmol) was added. The mixture was stirred at RT overnight. After completion, the mixture was quenched with aq sat. NH₄Cl (4 mL) and then diluted with water (4 mL), and extracted with DCM (5 mL×3). The combined organic layers were washed with water (10 mL), concentrated and purified by column chromatography on silica to give 2-chloro-4-(3-(methylamino)-1H-thieno[2,3-c]pyrazol-1-yl)pyrimidine (80 mg, 27%). ¹H NMR (400 MHz, CDCl₃): δ 8.45 (d, J=5.6 Hz, 1H), 7.56 (d, J=5.6 Hz, 1H), 7.12 (d, J=5.6 Hz, 1H), 6.96 (d, J=5.2 Hz, 1H), 3.13 (s, 3H). ESI-MS (m/z): 266.0 (M+H)⁺.

### A20. 2-Chloro-4-(5-chloro-3-(methylamino)-1H-thieno[2,3-c]pyrazol-1-yl) pyrimidine

To a solution of 2-chloro-4-(3-(methylamino)-1H-thieno[2,3-c]pyrazol-1-yl) pyrimidine (133 mg, 0.5 mmol, 1 eq) in a mixed solution of benzene and acetic acid (1:1, 1.4 mL) was added NCS (73.4 mg, 0.55 mmol, 1.1 eq). The mixture was heated to 70° C. and stirred for 2 hours. After completion, the mixture was poured into ice water (5 g), extracted with DCM (5 mL×2), the combined organic layers were washed with brine (5 mL), dried, concentrated and purified by silica column to give the desired product (75 mg, 50%). ¹H NMR (300 MHz, CDCl₃): δ 8.47 (d, J=5.4 Hz, 1H), 7.53 (d, J=5.4 Hz, 1H), 6.90 (s, 1H), 3.10 (s, 3H), 1.5-1.75 (brs, 1H). ESI-MS (m/z): 300.1 (M+H)⁺.

### A21. 2-Chloro-4-(1-methyl-1H-indol-4-yl) pyrimidine

### 4-Bromo-1-methyl-1H-indole

NaH (1.22 g, 51.02 mmol, 2.0 eq) was added portion wise to a stirred solution of 4-bromo-1H-indole (5.0 g, 25.51 mmol, 1.0 eq) in DMF (100 mL), at 0° C. The mixture was stirred for 30 min, and then CH₃I (9.0 g, 63.77 mmol, 2.5 eq) in DMF (20 mL) was added at 0° C. The reaction mixture was stirred at 0° C. for 3 h. TLC and LC-MS indicated completion, water (50 mL) was added and the mixture was extracted with EtOAc (2×50 mL), dried over sodium sulfate, concentrated and purified by silica column to give 4-bromo-1-methyl-1H-indole (3.2 g, 56%). ¹H NMR (300 MHz, CDCl₃): δ 7.31-7.27 (m, 2H), 7.14-7.12 (m, 2H), 6.56 (s, 1H, 3.82 (s, 3H). ESI-MS (m/z): 210.0 (M+H)⁺.

### 1-Methyl-4-(4,4,5,5-tetramethyl-1,3,2-dioxaborolan-2-yl)-1H-indole

A 250 mL flask was charged with 4-bromo-1-methyl-1H-indole (3.5 g, 16.66 mmol, 1.0 eq), bis(pinacolato)diboron (6.3 g, 24.99 mmol, 1.5 eq), KOAc (4.9 g, 49.98 mmol, 3.0 eq) and PdCl₂(dppf) CH₂Cl₂ complex (1.36 g, 1.66 mmol, 0.1 eq) under argon. Dry 1,4-Dioxane (70 mL) was added and the mixture was heated to 90° C. and stirred for 4 h. The reaction mixture was cooled, filtered through a silica gel, plug and the plug was washed with TBME (2×50 mL). The combined filtrates were washed with brine (3×50 mL), dried (Na₂SO₄), concentrated, and purified by silica column to give 1-methyl-4-(4,4,5,5-tetramethyl-1,3,2-dioxaborolan-2-yl)-1H-indole (3.0 g, 71%). ¹H NMR (300 MHz, CDCl₃): δ 7.70 (d, J=7.2 Hz, 1H), 7.48 (d, J=8.1 Hz, 1H), 7.30-7.27 (m, 1H), 7.10 (d, J=3.0 Hz, 1H), 7.04 (d, J=2.7 Hz, 1H), 3.83 (s, 3H), 1.45 (s, 12H). ESI-MS (m/z): 258.2 (M+H)⁺.

### 2-Chloro-4-(1-methyl-1H-indol-4-yl)pyrimidine

To a solution of 1-methyl-4-(4,4,5,5-tetramethyl-1,3,2-dioxaborolan-2-yl)-1H-indole (1.0 g, 3.89 mmol, 1.0 eq) in 1,4-dioxane/water (5:1, 12 mL) was added 2,4-dichloropyrimidine (700 mg, 4.69 mmol, 1.2 eq), Na₂CO₃ (1.23 g, 11.67 mmol, 3.0 eq), and (dppf)₂PdCl₂ (140 mg, 0.19 mmol, 0.05 eq) under argon. The mixture was purged with argon at room temperature for 10 min, refilled with argon, and stirred at 100° C. until TLC indicated completion. The reaction mixture was filtered through celite and concentrated to give a crude residue, which was purified on silica gel chromatography to give 2-chloro-4-(1-methyl-1H-indol-4-yl) pyrimidine (500 mg, 52%). ¹H NMR (300 MHz, DMSO): δ 8.78 (d, J=5.6 Hz, 1H), 8.12 (d, J=4.2 Hz, 1H), 7.84 (d, J=7.6 Hz, 1H), 7.73 (d, J=8.4 Hz, 1H), 7.56 (d, J=3.2 Hz, 1H), 7.34 (t, J=8.0 Hz, 1H), 7.13 (d, J=2.8 Hz, 1H), 3.88 (s, 1H). ESI-MS (m/z): 244.1 (M+H)⁺.

### A22. 2-Chloro-4-(7-cyano-1,3-dimethyl-1H-indol-5-yl)pyrimidine

### 5-Bromo-7-cyano-1,3-dimethyl-1H-indole

NaH (480 mg, 12 mmol, 1.2 eq) was added carefully to a cooled solution of 5-bromo-7-cyano-3-methyl-1H-indole (2.34 g, 10 mmol, 1.0 eq) in DMF (40 mL) under nitrogen and the mixture was stirred at 0° C. for 30 minutes. Then MeI (1.7 g, 12 mmol, 1.2 eq) was added drop wise. After addition, the mixture was stirred at this temperature for 30 minutes till completion. The mixture was poured into water (100 mL), extracted with EA (100 mL×3). The combined organic layers were washed with brine twice, dried over sodium sulfate, filtered and the filtrate was concentrated in vacuo to give a residue, which was purified by silica gel column chromatography to afford 5-bromo-7-cyano-1,3-dimethyl-1H-indole (1.6 g, 64%). ¹H NMR (300 MHz, CDCl₃): δ 7.85 (s, 1H), 7.58 (s, 1H), 6.86 (s, 1H), 4.04 (s, 3H), 2.26 (s, 3H).

### 7-Cyano-1,3-dimethyl-5-(4,4,5,5-tetramethyl-1,3,2-dioxaborolan-2-yl)-1H-indole

To a solution of 5-bromo-7-cyano-1,3-dimethyl-1H-indole (1.24 g, 5.0 mmol, 1.0 eq) in 1,4-dioxane (20 mL) were added bis(pinacolato)diboron (1.65 g, 6.5 mmol, 1.3 eq), KOAc (1.47 g, 15 mmol, 3.0 eq) and Pd(dppf)Cl₂DCM (412 mg, 0.5 mmol, 0.1 eq) under nitrogen. The mixture was purged with nitrogen 3 times and stirred at 90° C. for 2 hours. After cooling the mixture was concentrated and the residue was purified by chromatography on silica gel to give the title compound (940 mg, 63%). ¹H NMR (300 MHz, CDCl₃): δ 8.15 (s, 1H), 7.91 (s, 1H), 6.76 (s, 1H), 4.00 (s, 3H), 2.25 (s, 3H), 1.31 (s, 12H). ESI-MS (m/z): 297.2 (M+H)⁺.

### 2-Chloro-4-(7-cyano-1,3-dimethyl-1H-indol-5-yl)pyrimidine

To a solution of 7-cyano-1,3-dimethyl-5-(4,4,5,5-tetramethyl-1,3,2-dioxaborolan-2-yl)-1H-indole (592 mg, 2.0 mmol, 1.0 eq) in 1,4-dioxane (10 mL) and water (2 mL) were added 2,4-dichloropyrimidine (325.6 mg, 2.2 mmol, 1.1 eq), K₂CO₃ (828 mg, 6.0 mmol, 3.0 eq) and Pd(dppf)Cl₂DCM (164.7 mg, 0.068 mmol, 0.1 eq) under nitrogen. The mixture was bubbled with nitrogen for 10 minutes, then purged with nitrogen 3 times and stirred at 80° C. for 2.5 hours. After cooling the mixture was concentrated and the residue was purified by chromatography on silica gel to give 2-chloro-4-(7-cyano-1,3-dimethyl-1H-indol-5-yl)pyrimidine (310 mg, 55%). ¹H NMR (300 MHz, DMSO-d₆): δ 8.56 (d, J=5.2 Hz, 1H), 8.47 (s, 1H), 8.20 (s, 1H), 7.61 (d, J=5.2 Hz, 1H), 6.87 (s, 1H), 4.04 (s, 3H), 2.31 (s, 3H). ESI-MS (m/z): 283.1 (M+H)⁺.

### B1. N-(5-Amino-4-(difluoromethoxy)-2-((2-(dimethylamino)ethyl)(methyl) amino)phenyl)acrylamide

### 2-(Difluoromethoxy)-4-fluoro-1-nitrobenzene

To a solution of 5-fluoro-2-nitrophenol (20 g, 127 mmol, 1.0 eq) in DMF (200 mL) was added sodium chlorodifluoroacetate (28 g, 184 mmol, 1.5 eq) portion wise, then K₂CO₃ (32 g, 254 mmol, 2.0 eq) was added. The mixture was stirred at 90° C. for 2 hours. After completion, the mixture was quenched with water (200 mL), extracted with MTBE (150 mL×3), the combined organic layers were dried, concentrated and purified by silica column to give 2-(difluoromethoxy)-4-fluoro-1-nitrobenzene (20 g, 76%). ¹H NMR (300 MHz, CDCl₃): δ 8.06-8.01 (m, 1H), 7.27-7.08 (m, 2H), 6.66 (t, J=72.3 Hz, 1H).

### 2-(Difluoromethoxy)-4-fluoroaniline

To a solution of compound of 2-(difluoromethoxy)-4-fluoro-1-nitrobenzene (20 g, 96 mmol, 1.0 eq) in MeOH (200 mL) was added Pd/C (4 g), the mixture was stirred under 1 atm hydrogen atmosphere at room temperature overnight. LC-MS indicated starting material disappeared. The reaction mixture was filtered through celite and the filtrate was concentrated in vacuo to give the crude product 2-(difluoromethoxy)-4-fluoroaniline (16 g), which was used directly for the next step without further purification. ESI-MS (m/z): 177.9 (M+H)⁺.

### 2-(Difluoromethoxy)-4-fluoro-5-nitroaniline

2-(difluoromethoxy)-4-fluoro aniline (16 g, 8.5 mmol, 1.0 eq) was added portion wise to a cold solution of concentrated sulfuric acid (30 mL) at 0° C., after addition, potassium nitrate (10 g, 9.9 mmol, 1.1 eq) was added portion wise. The mixture was stirred at 0° C. for 2 h, LC-MS indicated starting material had disappeared, the reaction mixture was poured into ice water and neutralized to pH 9 by aq. sodium bicarbonate, extracted with MTBE (150 mL×3), the combined organic layers were dried over sodium sulfate, concentrated to give a crude residue, which was purified by silica gel column chromatography to give the desired product 2-(difluoromethoxy)-4-fluoro-5-nitroaniline (12 g, 60%). ¹H NMR (300 MHz, CDCl₃): δ 7.49 (d, J=6.9 Hz, 1H), 7.02 (d, J=10.8 Hz, 1H), 6.62 (t, J=72.0 Hz, 1H), 4.12 (br, 2H). ESI-MS (m/z): 222.9 (M+H)⁺.

### N-t-Butoxycarbonyl-2-(difluoromethoxy)-4-fluoro-5-nitroaniline

To a solution of 2-(difluoromethoxy)-4-fluoro-5-nitroaniline (12 g, 54 mmol, 1.0 eq) in DCM (120 mL) was added DIPEA (10.4 g, 81 mmol, 1.5 eq) and DMAP (0.56 g, 5.4 mmol, 0.1 eq), then a solution of (Boc)₂O (14.14 g, 64.8 mmol, 1.2 eq) in DCM (20 mL) was added dropwise. The reaction was stirred at room temperature overnight, LC-MS indicated that the starting material had disappeared. The solvents were removed under reduced pressure to give a crude residue, which was purified by silica gel column chromatography to give the desired product N-t-butoxycarbonyl-2-(difluoromethoxy)-4-fluoro-5-nitroaniline (8.7 g, 50%)._ESI-MS (m/z): 320.8 (M−H)⁻.

### N-t-Butoxycarbonyl-2-(difluoromethoxy)-4-((2-(dimethylamino)ethyl)(methyl) amino)-5-nitroaniline

To a solution of N-t-butoxycarbonyl-2-(difluoromethoxy)-4-fluoro-5-nitroaniline (8.7 g, 76.1 mmol, 1.0 eq) in EtOH (40 mL) was add DIPEA (11.8 g, 91.4 mmol, 1.2 eq) and N,N,N′-trimethylethane-1,2-diamine (8.7 g, 83.5 mmol, 1.1 eq), the mixture was heated to 60° C. and stirred overnight. LC-MS and TLC indicated that the staring material had disappeared. The reaction was concentrated to give a crude residue N-t-butoxycarbonyl-2-(difluoromethoxy)-4-((2-(dimethylamino)ethyl)(methyl) amino)-5-nitroaniline (14 g), which was used directly for the next step without further purification. ESI-MS (m/z): 404.9 (M+H)⁺.

### 5-Amino-(1,N-t-butoxycarbonyl)-2-(difluoromethoxy)-4-((2-(dimethylamino) ethyl)(methyl)amino)aniline

To a solution of N-t-butoxycarbonyl-2-(difluoromethoxy)-4-((2-(dimethylamino)ethyl)(methyl) amino)-5-nitroaniline (14 g, crude) in MeOH (200 mL) was added Pd/C (4 g), the mixture was stirred under 1 atm hydrogen atmosphere at room temperature overnight. LC-MS indicated starting material disappeared. The reaction mixture was filtered through celite and the filtrate was concentrated in vacuo to give the crude product 5-amino-(1,N-t-butoxycarbonyl)-2-(difluoromethoxy)-4-((2-(dimethylamino) ethyl)(methyl)amino)aniline (11 g), which was used directly for the next step without further purification. ESI-MS (m/z): 374.9 (M+H)⁺.

### 5-Acrylamino-(1,N-t-butoxycarbonyl)-2-(difluoromethoxy)-4-((2-(dimethylamino)ethyl)(methyl)amino)aniline

Acryloyl chloride (690 mg, 7.6 mmol, 1.5 eq) was added dropwise to a solution of 5-amino-(1,N-t-butoxycarbonyl)-2-(difluoromethoxy)-4-((2-(dimethylamino) ethyl)(methyl)amino)aniline (1.5 g, 5 mmol, 1.0 eq) and DIPEA (780 mg, 6 mmol, 1.2 eq) in THF (30 mL) at 0° C. The resulting mixture was stirred for 1 h. The reaction mixture was quenched with sat. NaHCO₃ (20 mL), extracted with EA (30 mL×3) and the organic extract was washed with brine (30 mL), dried over sodium sulfate, and concentrated affording the desired product 5-acrylamino-(1,N-t-butoxycarbonyl)-2-(difluoromethoxy)-4-((2-(dimethylamino)ethyl)(methyl)amino)aniline (500 mg, crude) which was used in next step directly without further purification. ESI-MS (m/z): 428.9 (M+H)⁺.

### N-(5-Amino-4-(difluoromethoxy)-2-((2-(dimethylamino)ethyl)(methyl)amino) phenyl)acrylamide

To a solution of 5-acrylamino-(1,N-t-butoxycarbonyl)-2-(difluoromethoxy)-4-((2-(dimethylamino)ethyl)(methyl)amino) aniline (500 mg, crude) in DCM (5 mL) was added TFA (3 mL), the mixture was heated to reflux overnight. TLC, LC-MS indicated starting material had disappeared. The reaction mixture was neutralized by sat. NaHCO₃ to pH=9, extracted with DCM (10 mL×3) and the combined organic extracts were washed with brine (20 mL), dried over sodium sulfate, concentrated to give a crude residue, which was purified by silica gel column chromatography to give N-(5-amino-4-(difluoromethoxy)-2-((2-(dimethylamino)ethyl) (methyl)amino)phenyl)acrylamide (200 mg, 12.2% on 2 steps). ¹H NMR (300 MHz, DMSO-d₆): δ 10.07 (brs, 1H), 7.77 (s, 1H), 6.97 (s, 1H), 6.96 (t, J=75 Hz, 1H), 6.43-6.20 (m, 2H), 5.77-5.74 (m, 1H), 5.01 (s, 2H), 2.80-2.75 (m, 2H), 2.64 (s, 3H), 2.34-2.30 (m, 2H), 2.25 (s, 6H). ESI-MS (m/z): 328.9 (M+H)⁺.

### B2. N-(5-Amino-4-(2,2-difluoroethoxy)-2-((2-(dimethylamino)ethyl)(methyl) amino)phenyl)acrylamide

### 2-(2,2-Difluoroethoxy)-4-fluoro-1-nitrobenzene

To a solution of 2,4-difluoro-1-nitrobenzene (30.0 g, 1.89 mmol, 1.0 eq) and 2,2-difluoroethan-1-ol (20.1 g, 2.44 mol, 1.3 eq) in toluene (60 mL) was added sodium hydroxide (9.0 g, 2.26 mmol, 1.2 eq) in portions over 30 min to keep the temperature between 30 and 40° C. The reaction was stirred at 45° C. until 2,4-difluoro-1-nitrobenzene had disappeared. After cooling, water (60 mL) and 2.5 N H₂SO₄ (30 mL) to neutralize pH to 5 were added, and the organic layer was separated. The aqueous layer was extracted with EtOAc (30 mL×2). The combined organic layers were washed with sat. NaCl (10 mL), dried over Na₂SO₄, filtered and concentrated to give a crude residue, which was purified by column chromatography to give the desired product 2-(2,2-difluoroethoxy)-4-fluoro-1-nitrobenzene (32 g, 78%) as a yellow solid. ¹H NMR (300 MHz, CDCl₃): δ 8.00 (t, J=7.2 Hz, 1H), 6.88-6.80 (m, 2H), 6.19 (t, J=54.6 Hz, 1H), 4.36-4.12 (m, 2H). ESI-MS (m/z): 221.8 (M+H)⁺.

### 2-(2,2-Difluoroethoxy)-4-fluoroaniline

To a solution of 2-(2,2-difluoroethoxy)-4-fluoro-1-nitrobenzene (32 g, 145 mmol, 1.0 eq) in MeOH (320 mL) was added Pd/C (10%, 6.4 g, 0.4 eq). The reaction was stirred at room temperature under 1 atm hydrogen atmosphere overnight. The reaction mixture was filtered through celite and concentrated to give 2-(2,2-difluoroethoxy)-4-fluoroaniline as a red solid (26.0 g, 95%). ¹HNMR (300 MHz, CDCl₃): δ 7.01-6.97 (m, 1H), 6.70-6.62 (m, 2H), 6.20 (tt, J_{d}=1.5 Hz 54.6 Hz, 1H), 4.32 (dt J_{d}=1.5 Hz, Jₜ=12.3 Hz, 2H). ESI-MS (m/z): 191.9 (M+H)⁺.

### 2-(2,2-Difluoroethoxy)-4-fluoro-5-nitroaniline

2-(2,2-difluoroethoxy)-4-fluoroaniline (26 g, 136 mmol, 1.0 eq) was added portion wise at 0° C. to conc. H₂SO₄ (60 mL). Then KNO₃ (17.2 g, 164 mmol, 1.2 eq) was added in portions, and when the addition was completed, the reaction was warmed to rt. After LCMS indicated starting material disappeared, the reaction mixture was poured into ice-water and neutralized by Na₂CO₃, extracted with MBTE, dried over Na₂SO₄, filtered, concentrated in vacuo, and then purified by column chromatography to give the desired product 2-(2,2-difluoroethoxy)-4-fluoro-5-nitroaniline (15.2 g, 47%). ¹H NMR (300 MHz, CDCl₃) δ 7.45 (d, J=4.2 Hz, 1H), 6.67 (d, J=11.7 Hz, 1H), 6.18 (sl br t, J=54.3 Hz, 1H), 4.29 (sl br t, Jₜ=12.6 Hz, 1H), 4.10-3.85 (br, 2H). ESI-MS (m/z): 236.9 (M+H)⁺.

### N-t-Butoxycarbonyl-2-(2,2-difluoroethoxy)-4-fluoro-5-nitroaniline

To a solution of 2-(2,2-difluoroethoxy)-4-fluoro-5-nitroaniline (15.2 g, 64.4 mmol, 1.0 eq), DMAP (0.786 g, 6.44 mmol, 0.1 eq) and DIPEA (12.45 g, 96.6 mmol, 1.5 eq) in DCM (150 mL) was added (Boc)₂O (15.45 g, 70.8 mmol, 1.1 eq), the reaction mixture was stirred at room temperature overnight. TLC and LCMS indicated no starting materials remained, the reaction mixture concentrated in vacuo and the residue was purified by column chromatography to afford N-t-butoxycarbonyl-2-(2,2-difluoroethoxy)-4-fluoro-5-nitroaniline (6.6 g, 21%). ¹HNMR (300 MHz, CDCl₃) δ 8.98 (br, 1H), 6.86 (sl br s, 1H), 6.75 (d, J=11.4 Hz, 1H), 6.21 (sl br t, J=55.2 Hz, 1H), 4.34 (dₜ, J_{d}=2.7 Hz, Jₜ=12.3 Hz, 2H), 1.56 (s, 9H). ESI-MS (m/z): 334.8 (M−H)⁻.

### N-t-Butoxycarbonyl-2-(2,2-Difluoroethoxy)-4-((2-(dimethylamino)ethyl) (methyl)amino)-5-nitroaniline

To a solution of N-t-butoxycarbonyl-2-(2,2-difluoroethoxy)-4-fluoro-5-nitroaniline (6.6 g, 19.6 mmol, 1.0 eq) in EtOH (130 mL) was added DIPEA (2.46 g, 19.6 mmol, 1.0 eq) and N¹,N¹, N²-trimethylethane-1,2-diamine (2.5 g, 23.5 mmol, 1.2 eq), and the reaction mixture was heated to 60° C. overnight. LCMS indicated the reaction was completed, so the mixture was cooled to rt, and concentrated in vacuo to give a crude residue, which was purified by column chromatography to give the desired product N-t-butoxycarbonyl-2-(2,2-Difluoroethoxy)-4-((2-(dimethylamino)ethyl) (methyl)amino)-5-nitroaniline (7.5 g, 90%) as a red oil. ¹H NMR (300 MHz, CDCl₃) δ 8.60 (br, 1H), 6.83 (s, 1H), 6.80 (1H, s), 6.19 sl br (t, J=54.6 Hz, 1H), 4.34 (sl br t J=13.2 Hz, 2H), 3.52-3.31 (m, 2H), 2.90 (s, 3H), 2.84-2.67 (m, 2H), 2.38 (s, 6H), 1.54 (s, 9H). ESI-MS (m/z): 418.8 (M+H)⁺.

### 5-Amino-(1,N-t-butoxycarbonyl)-2-(2,2-difluoroethoxy)-4-((2-(dimethylamino) ethyl)(methyl)amino)aniline

To a solution of N-t-butoxycarbonyl-2-(2,2-Difluoroethoxy)-4-((2-(dimethylamino)ethyl)(methyl)amino)-5-nitroaniline (7.5 g, 17.9 mmol, 1.0 eq) in MeOH (80 mL) was added Pd/C (10 wt %, 5.6 g, 3.0 eq). The reaction was stirred at rt under 1 atm hydrogen atmosphere overnight. The reaction mixture was filtered through celite and concentrated to give 5-amino-(1,N-t-butoxycarbonyl)-2-(2,2-difluoroethoxy)-4-((2-(dimethylamino)ethyl)(methyl)amino) aniline (6.6 g, 96%) as a red solid. ¹H NMR (300 MHz, CDCl₃): δ 7.53 (s, 1H), 6.86 (s, 1H), 6.65 (s, 1H), 6.08 (t, J=54.0 Hz, 1H), 4.14 (t, J=13.2 Hz, 2H), 3.00-2.80 (m, 2H), 2.63 (s, 3H), 2.44-2.30 (m, 2H), 2.22 (s, 6H), 1.53 (s, 9H). ESI-MS (m/z): 388.9 (M+H)⁺.

### 5-Acrylamido-(1,N-t-butoxycarbonyl)-2-(2,2-difluoroethoxy)-4-((2-(dimethylamino) ethyl)(methyl)amino)aniline

To a solution of 5-amino-(1,N-t-butoxycarbonyl)-2-(2,2-difluoroethoxy)-4-((2-(dimethylamino)ethyl)(methyl)amino) aniline (6.6 g, 17.0 mmol, 1.0 eq) and DIPEA (2.45 g, 18.9 mmol, 1.1 eq) in THF (66 mL) at 0° C. was add drop wise a solution of acryloyl chloride (1.69 g, 18.9 mmol, 1.1 eq) in THF (5 mL). After addition, the mixture was stirred at 0° C. for 10 min and warmed to rt. LCMS indicated starting material disappeared, the reaction was quenched with water (30 mL), extracted with EA and washed with sat. NaHCO₃ and brine, dried over Na₂SO₄, filtered and concentrated to give a crude residue, which was purified by column chromatography to give the desired product 5-acrylamido-(1,N-t-butoxycarbonyl)-2-(2,2-difluoroethoxy)-4-((2-(dimethylamino)ethyl)(methyl)amino) aniline (2.5 g, 33%). ESI-MS (m/z): 443.3 (M+H)⁺.

### N-(5-Amino-4-(2,2-difluoroethoxy)-2-((2-(dimethylamino)ethyl)(methyl)amino) phenyl)acrylamide

To a solution of 5-acrylamido-(1,N-t-butoxycarbonyl)-2-(2,2-difluoroethoxy)-4-((2-(dimethylamino)ethyl)(methyl)amino) aniline (2.9 g, 6.56 mmol, 1.0 eq) in DCM (20 mL) was added TFA (10 mL), and then the mixture was heated to reflux. After TLC and LCMS indicated starting material disappeared, the reaction mixture was concentrated in vacuo at rt to remove most of TFA and DCM, neutralized by NaHCO₃ to pH=7, extracted by DCM/MeOH (10:1), dried over Na₂SO₄, filtered and concentrated in vacuo to give the crude residue, which was purified by column chromatography to afford the desired product N-(5-amino-4-(2,2-difluoroethoxy)-2-((2-(dimethylamino)ethyl)(methyl)amino) phenyl)acrylamide (1.4 g, 64%) as a gray solid. ¹H NMR (300 MHz, CDCl₃): δ 9.13 (s, 1H), 6.78 (s, 1H), 6.72 (s, 1H), 6.47-6.42 (m, 1H), 6.12 (tt, J=55.2 Hz, 3.9 Hz, 1H), 5.72-5.67 (m, 1H), 4.21 (dt, J_{d}=4.0 Hz, Jₜ=13.0 Hz, 2H), 3.00-2.96 (m, 2H), 2.68 (s, 3H), 2.46-2.42 (m, 8H). ESI-MS (m/z): 343.2 (M+H)⁺.

### Example 1. N-(5-((4-(3-(Dimethylamino)-6-methyl-1H-pyrazolo[4,3-c]pyridin-1-yl)pyrimidin-2-yl)amino)-2-((2-(dimethylamino)ethyl)(methyl)amino)-4-methoxyphenyl)acrylamide

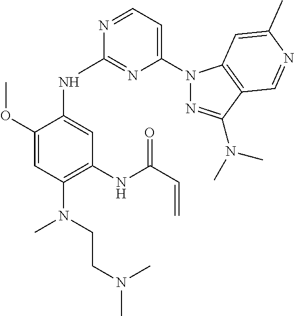 [SMILES: C=CC(=O)Nc1cc(Nc2nccc(-n3nc(N(C)C)c4cnc(C)cc43)n2)c(OC)cc1N(C)CCN(C)C]

2-Chloro-4-(N,N,6-trimethyl-pyrazolo[4,3-c]pyridin-3-amine-1-yl)pyrimidine (120 mg, 0.42 mmol, 1.0 eq), N-(5-amino-2-((2-(dimethylamino)ethyl)(methyl)amino)-4-methoxyphenyl)acrylamide (134 mg, 0.46 mmol, 1.1 eq) and 2-pentanol (2 mL) and p-TsOH.H₂O (87 mg, 0.46 mmol, 1.1 eq) were sealed in a 10 mL Schlenk tube. The mixture was stirred at 120° C. for 2 h. After completion, the mixture was cooled to RT and diluted with sat. NaHCO₃ (10 mL) and DCM/MeOH (10/1, 20 mL), the organic layer was separated and the aqueous layer was extracted with DCM (5 mL×2). The combined organic layers were washed with NaHCO₃ (20 mL×2) and brine (20 mL), dried, concentrated and purified by prep-HPLC affording N-(5-((4-(3-(dimethylamino)-6-methyl-1H-pyrazolo[4,3-c]pyridin-1-yl)pyrimidin-2-yl)amino)-2-((2-(dimethylamino)ethyl)(methyl)amino)-4-methoxyphenyl)acrylamide (6 mg, 2.7%). ¹H NMR (300 MHz, DMSO-d₆): δ 9.76 (br, 1H), 9.44 (m, 1H), 8.99 (s, 1H), 8.42-8.40 (m, 2H), 7.50-7.33 (m, 2H), 6.83-6.65 (m, 2H), 6.33-6.27 (m, 1H), 5.66-5.64 (m, 1H), 3.90 (s, 3H), 3.18 (s, 6H), 3.09-3.07 (m, 2H), 2.82-2.80 (m, 5H), 2.56 (s, 3H), 2.50 (s, 6H). ESI-MS (m/z): 544.8 (M+H)⁺.

### Example 2. N-(5-((4-(7-Cyano-1,3-dimethyl-1H-indol-5-yl)pyrimidin-2-yl)amino)-2-((2-(dimethylamino)ethyl)(methyl)amino)-4-methoxyphenyl)acrylamide

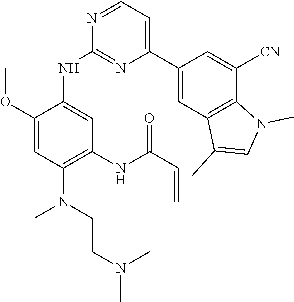 [SMILES: C=CC(=O)Nc1cc(Nc2nccc(-c3cc(C#N)c4c(c3)c(C)cn4C)n2)c(OC)cc1N(C)CCN(C)C]

To a solution of 2-chloro-4-(7-cyano-1,3-dimethyl-1H-indol-5-yl)pyrimidine (164 mg, 0.58 mmol, 1.0 eq) and N-(5-amino-2-((2-(dimethylamino)ethyl)(methyl) amino)-4-methoxyphenyl)acrylamide (170 mg, 0.58 mmol, 1.0 eq) in 2-pentanol (4 mL) was added p-toluenesulfonic acid monohydrate (123 mg, 0.64 mmol, 1.1 eq). The mixture was heated to 120° C. for 5 h in a 10 mL Schlenk tube. After cooling down to RT, the mixture was poured into water (10 mL), extracted with DCM/MeOH=10:1 (10 mL×3), the combined organic layers were washed with brine (10 mL), dried over sodium sulfate, concentrated and purified by silica column affording N-(5-((4-(7-cyano-1,3-dimethyl-1H-indol-5-yl)pyrimidin-2-yl)amino)-2-((2-(dimethylamino)ethyl)(methyl)amino)-4-methoxy phenyl)acrylamide (48 mg, 15%). ¹H NMR (300 MHz, DMSO-d₆): δ 10.19 (br, 1H), 9.07 (s, 1H), 8.71 (s, 1H), 8.51-8.49 (m, 2H), 8.20 (s, 1H), 7.59-7.57 (m, 1H), 7.32 (s, 1H), 7.04 (s, 1H), 6.40-6.34 (m, 1H), 6.27-6.21 (m, 1H), 5.75-5.72 (m, 1H), 4.04 (s, 3H), 3.85 (s, 3H), 2.89-2.87 (m, 2H), 2.71 (s, 3H), 2.34-2.32 (m, 2H), 2.23 (s, 3H), 2.17 (s, 6H). ESI-MS (m/z): 538.8 (M+H)⁺. HPLC: 94.8%.

### Example 3. N-(5-((4-(7-Cyano-3-methyl-1H-pyrrolo[2,3-c]pyridin-5-yl)pyrimidin-2-yl)amino)-2-((2-(dimethylamino)ethyl)(methyl)amino)-4-methoxyphenyl)acryl amide

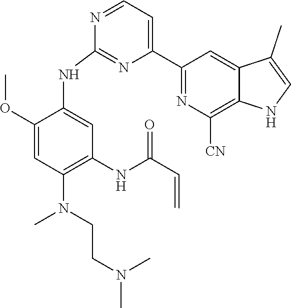 [SMILES: C=CC(=O)Nc1cc(Nc2nccc(-c3cc4c(C)c[nH]c4c(C#N)n3)n2)c(OC)cc1N(C)CCN(C)C]

To a 10 mL microwave tube were added 2-chloro-4-(1,N-(tert-butoxycarbonyl)-7-cyano-3-methyl-pyrrolo[2,3-c]pyridin-4-yl) pyrimidine (90 mg, 0.24 mmol, 1.0 eq), N-(5-amino-2-((2-(dimethylamino)ethyl)(methyl)amino)-4-methoxyphenyl)acrylamide (78 mg, 0.27 mmol, 1.1 eq), 2-pentanol (2 mL) and p-TsOH.H₂O (51 mg, 0.27 mmol, 1.1 eq). The mixture was stirred at 170° C. under microwave for 1 h. After completion, the mixture was cooled to RT and diluted with sat. NaHCO₃ (10 mL) and DCM/MeOH (10/1, 11 mL), the organic layer was separated and the aqueous layer was extracted with DCM/MeOH (5 mL×2). The combined organic layers were washed with NaHCO₃ (10 mL) and brine (10 mL), dried, concentrated and purified by prep-HPLC affording N-(5-((4-(7-cyano-3-methyl-1H-pyrrolo[2,3-c]pyridin-5-yl)pyrimidin-2-yl)amino)-2-((2-(dimethylamino)ethyl)(methyl)amino)-4-methoxyphenyl)acrylamide (6 mg, 4.8%). ¹H NMR (300 MHz, DMSO-d₆): δ 12.48 (br, 1H), 10.20 (s, 1H), 9.21 (s, 1H), 9.01 (s, 1H), 8.56 (d, J=4.8 Hz, 1H), 8.17 (s, 1H), 7.72 (d, J=4.8 Hz, 1H), 7.64 (s, 1H), 7.05 (s, 1H), 6.45-6.36 (m, 1H), 6.20-6.14 (m, 1H), 5.73 (m, 1H), 3.87 (s, 3H), 2.89-2.87 (m, 2H), 2.72 (s, 3H), 2.50-2.48 (m, 2H), 2.30 (s, 3H), 2.22 (s, 6H). ESI-MS (m/z): 526.2 (M+H)⁺. HPLC: 98.0%.

### Example 4. N-(5-((4-(6-Cyano-1-methyl-1H-indol-4-yl)pyrimidin-2-yl)amino)-2-((2-(dimethylamino)ethyl)(methyl)amino)-4-methoxyphenyl)acrylamide

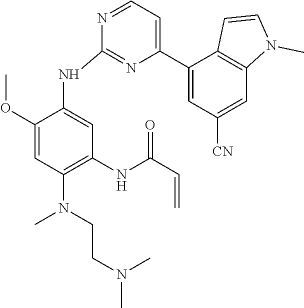 [SMILES: C=CC(=O)Nc1cc(Nc2nccc(-c3cc(C#N)cc4c3ccn4C)n2)c(OC)cc1N(C)CCN(C)C]

To a 10 mL microwave tube were added 2-chloro-4-(6-cyano-1-methyl-1H-indol-4-yl) pyrimidine (269 mg, 1.0 mmol, 1.0 eq), N-(5-amino-2-((2-(dimethylamino)ethyl)(methyl)amino)-4-methoxyphenyl)acrylamide (321 mg, 1.1 mmol, 1.1 eq) and 2-pentanol (5 mL) and p-TsOH.H₂O (175 mg, 1.0 mmol, 1.0 eq). The mixture was stirred at 150° C. in microwave for 2 h. After completion, the mixture was cooled to RT and diluted with sat. NaHCO₃ (10 mL) and DCM/MeOH (10/1, 20 mL), the organic layer was separated and the aqueous layer was extracted with DCM (5 mL×2). The combined organic layers were washed with NaHCO₃ (20 mL×2) and brine (20 mL), dried, concentrated and purified by prep-HPLC affording N-(5-((4-(6-cyano-1-methyl-1H-indol-4-yl)pyrimidin-2-yl)amino)-2-((2-(dimethylamino)ethyl) (methyl)amino)-4-methoxyphenyl)acrylamide (38 mg, 5%). ¹H NMR (300 MHz, DMSO-d₆): δ 10.11 (br, 1H), 8.81 (s, 1H), 8.51 (d, J=4.8 Hz, 1H), 8.39 (s, 1H), 8.23 (s, 1H), 8.11 (s, 1H), 7.69 (s, 1H), 7.42 (d, J=4.5 Hz, 1H), 7.10 (s, 1H), 7.02 (s, 1H), 6.24-6.38 (m, 2H), 5.74 (d, J=9.0 Hz, 1H), 3.91 (s, 3H), 3.81 (s, 3H), 2.88-2.99 (m, 2H), 2.79 (s, 3H), 2.13-2.33 (m, 2H), 2.22 (s, 6H). ESI-MS (m/z): 525.3 (M+H)⁺.

### Example 5. N-(5-((4-(3-(Dimethylamino)-1H-thieno[2,3-c]pyrazol-1-yl)pyrimidin-2-yl)amino)-2-((2-(dimethylamino)ethyl)(methyl)amino)-4-methoxyphenyl) acrylamide

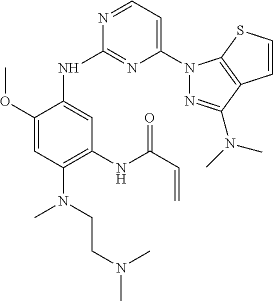 [SMILES: C=CC(=O)Nc1cc(Nc2nccc(-n3nc(N(C)C)c4ccsc43)n2)c(OC)cc1N(C)CCN(C)C]

To a 100 mL four-neck flask (10 mL Shlenk tube?) were added 2-chloro-4-(3-(N,N-dimethylamino)-1H-thieno[2,3-c]pyrazol-1-yl)pyrimidine (80 mg, 0.287 mmol, 1.0 eq), N-(5-amino-2-((2-(dimethylamino)ethyl)(methyl)amino)-4-methoxyphenyl) acrylamide (92 mg, 0.315 mmol, 1.1 eq) and 2-pentanol (2 mL) and TsOH.H₂O (54 mg, 0.315 mmol, 1.1 eq). The mixture was stirred at 120° C. for 2 h. After completion, the mixture was cooled to RT and diluted with water (3 mL) and DCM/MeOH (10/1, 4 mL), the organic layer was separated and the aqueous layer was extracted with DCM (5 mL×2). The combined organic layers were washed with NaHCO₃ (5 mL×2) and brine (5 mL), the combined organic layers were dried, concentrated and purified by prep-HPLC affording N-(5-((4-(3-(dimethylamino)-1H-thieno[2,3-c]pyrazol-1-yl)pyrimidin-2-yl)amino)-2-((2-(dimethylamino)ethyl)(methyl)amino)-4-methoxyphenyl) acrylamide (26 mg, 17%). ¹H NMR (300 MHz, DMSO-d₆): δ 10.20 (s, 1H), 8.44 (s, 2H), 8.32 (d, J=2.7 Hz, 1H), 7.21-7.10 (m, 1H), 7.08-7.02 (m, 2H), 6.94 (d, J=2.7 Hz, 1H), 6.37-6.33 (m, 1H), 6.20-6.14 (m, 1H), 5.73-5.70 (m, 1H), 3.76 (s, 3H), 3.06 (s, 6H), 2.91-2.90 (m, 2H), 2.74 (s, 3H), 2.34-2.33 (m, 2H), 2.22 (s, 6H). ESI-MS (m/z): 535.8 (M+H)⁺.

### Example 6. N-(5-((4-(5-Chloro-3-(dimethylamino)-1H-thieno[2,3-c]pyrazol-1-yl)pyrimidin-2-yl)amino)-2-((2-(dimethylamino)ethyl)(methyl)amino)-4-methoxy phenyl)acrylamide

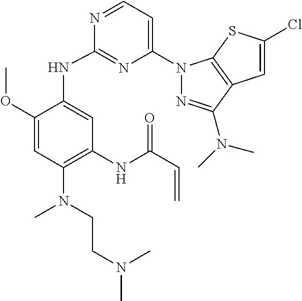 [SMILES: C=CC(=O)Nc1cc(Nc2nccc(-n3nc(N(C)C)c4cc(Cl)sc43)n2)c(OC)cc1N(C)CCN(C)C]

To a solution of 2-chloro-4-(3-(N,N-dimethylamino)-5-chloro-1H-thieno[2,3-c]pyrazol-1-yl)pyrimidine (78.3 mg, 0.25 mmol, 1.0 eq) and N-(5-amino-2-((2-(dimethylamino)ethyl)(methyl)amino)-4-methoxyphenyl) acrylamide (80.4 mg, 0.275 mmol, 1.1 eq) in 2-pentanol (2 mL) was added p-TsOH.H₂O (52.3 mg, 0.275 mmol, 1.1 eq). The mixture was heated in a Schlenk tube to 140° C. under microwave for 30 min. After completion, the mixture was cooled to RT and diluted with sat. NaHCO₃ (10 mL) and DCM/MeOH (10/1, 20 mL), the organic layer was separated and the aqueous layer was extracted with DCM (5 mL×2). The combined organic layers were washed with NaHCO₃ (20 mL×2) and brine (20 mL), dried, concentrated and purified by prep-HPLC affording N-(5-((4-(5-Chloro-3-(dimethylamino)-1H-thieno[2,3-c]pyrazol-1-yl)pyrimidin-2-yl) amino)-2-((2-(dimethylamino)ethyl)(methyl)amino)-4-methoxyphenyl)acrylamide (28 mg, 19%). ¹H NMR (300 MHz, DMSO-d₆): δ 10.16 (br, 1H), 8.71 (s, 1H), 8.35 (m, 2H), 7.42 (s, 1H), 7.05 (s, 1H), 6.90 (s, 1H), 6.38-6.35 (m, 1H), 6.20-6.15 (m, 1H), 5.77-5.70 (m, 1H), 3.72 (s, 3H), 3.03 (s, 6H), 2.94-2.92 (m, 2H), 2.75 (s, 3H), 2.40-2.35 (m, 2H), 2.24 (s, 6H). ESI-MS (m/z): 570.2 (M+H)⁺.

### Example 7. N-(5-((4-(4-Cyano-1-methyl-1H-indol-6-yl)pyrimidin-2-yl)amino)-2-((2-(dimethylamino)ethyl)(methyl)amino)-4-methoxyphenyl)acrylamide

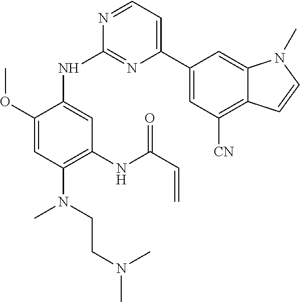 [SMILES: C=CC(=O)Nc1cc(Nc2nccc(-c3cc(C#N)c4ccn(C)c4c3)n2)c(OC)cc1N(C)CCN(C)C]

To a 10 mL microwave tube were added 2-chloro-4-(4-cyano-1-methyl-1H-indol-6-yl)pyrimidine (269 mg, 1.0 mmol, 1.0 eq), N-(5-amino-2-((2-(dimethylamino) ethyl)(methyl)amino)-4-methoxyphenyl) acrylamide (321 mg, 1.1 mmol, 1.1 eq) and 2-pentanol (5 mL) and p-TsOH.H₂O (175 mg, 1.0 mmol, 1.0 eq). The mixture was stirred at 150° C. in microwave for 2 h. After completion, the mixture was cooled to RT and diluted with sat. NaHCO₃ (10 mL) and DCM/MeOH (10/1, 20 mL), the organic layer was separated and the aqueous layer was extracted with DCM (5 mL×2). The combined organic layers were washed with NaHCO₃ (20 mL×2) and brine (20 mL), dried, concentrated and purified by prep-HPLC affording N-(5-((4-(4-cyano-1-methyl-1H-indol-6-yl)pyrimidin-2-yl) amino)-2-((2-(dimethylamino)ethyl)(methyl)amino)-4-methoxyphenyl)acrylamide (38 mg, 5%). ¹H NMR (300 MHz, DMSO-d₆): δ 10.21 (s, 1H), 9.18 (s, 1H), 8.70 (s, 1H), 8.49-8.54 (m, 2H), 8.17 (s, 1H), 7.79 (s, 1H), 7.62 (d, J=4.5 Hz, 1H), 7.05 (s, 1H), 6.64 (s, 1H), 6.23-6.43 (m, 2H), 5.75 (d, J=9.0 Hz, 1H), 3.94 (s, 3H), 3.87 (s, 3H), 2.88-2.86 (m, 2H), 2.71 (s, 3H), 2.32-3.30 (m, 2H), 2.22 (s, 6H). ESI-MS (m/z): 525.3 (M+H)⁺.

### Example 8 (Comparative). N-(2-((2-(Dimethylamino)ethyl)(methyl)amino)-4-methoxy-5-((4-(3-methoxy-1H-indazol-1-yl)pyrimidin-2-yl)amino)phenyl)acrylamide

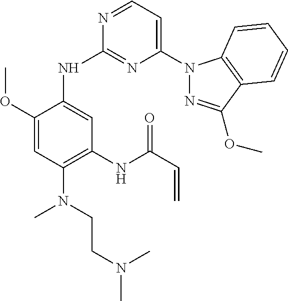 [SMILES: C=CC(=O)Nc1cc(Nc2nccc(-n3nc(OC)c4ccccc43)n2)c(OC)cc1N(C)CCN(C)C]

To a 10 mL Schlenk tube were added 2-chloro-4-(3-methoxy-1H-indazol-1-yl) pyrimidine (200 mg, 0.77 mmol, 1.0 eq), N-(5-amino-2-((2-(dimethylamino) ethyl)(methyl)amino)-4-methoxyphenyl)acrylamide (248 mg, 0.85 mmol, 1.1 eq) and 2-pentanol (3 mL) and p-TsOH.H₂O (160 mg, 0.85 mmol, 1.1 eq). The mixture was stirred at 120° C. for 2 h. After completion, the mixture was cooled to RT and diluted with sat. NaHCO₃ (10 mL) and DCM/MeOH (10/1, 20 mL), the organic layer was separated and the aqueous layer was extracted with DCM (5 mL×2). The combined organic layers were washed with NaHCO₃ (20 mL×2) and brine (20 mL), dried, concentrated and purified by prep-HPLC affording N-(2-((2-(dimethylamino) ethyl)(methyl)amino)-4-methoxy-5-((4-(3-methoxy-1H-indazol-1-yl)pyrimidin-2-yl)amino)phenyl)acrylamide (53 mg, 13%). ¹H NMR (300 MHz, DMSO-d₆): δ 10.08 (br, 1H), 8.71 (s, 1H), 8.46-8.45 (m, 2H), 8.34 (d, J=5.7 Hz, 1H), 7.69 (d, J=7.5 Hz, 1H), 7.37-7.35 (m, 1H), 7.28-7.26 (m, 1H), 7.08-7.05 (m, 2H), 6.47-6.42 (m, 1H), 6.20-6.15 (m, 1H), 5.74-5.71 (m, 1H), 4.13 (s, 3H), 3.76 (s, 3H), 2.96-2.94 (m, 2H), 2.75 (s, 3H), 2.46-2.30 (m, 2H), 2.28 (s, 6H). ESI-MS (m/z): 517.2 (M+H)⁺.

### Example 9. N-(5-((4-(6-Cyano-1-methyl-1H-indazol-4-yl)pyrimidin-2-yl)amino)-2-((2-(dimethylamino)ethyl)(methyl)amino)-4-methoxyphenyl)acrylamide

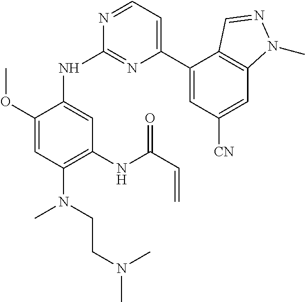 [SMILES: C=CC(=O)Nc1cc(Nc2nccc(-c3cc(C#N)cc4c3cnn4C)n2)c(OC)cc1N(C)CCN(C)C]

To a 10 mL microwave tube were added 2-chloro-4-(6-cyano-1-methyl-1H-indazol-4-yl)pyrimidine (269 mg, 1.0 mmol, 1.0 eq), N-(5-amino-2-((2-(dimethylamino)ethyl)(methyl)amino)-4-methoxyphenyl)acrylamide (321 mg, 1.1 mmol, 1.1 eq) and 2-pentanol (5 mL) and p-TsOH.H₂O (175 mg, 1.0 mmol, 1.0 eq). The mixture was stirred at 150° C. in a microwave for 2 h. After completion, the mixture was cooled to RT and diluted with sat. NaHCO₃ (10 mL) and DCM/MeOH (10/1, 20 mL), the organic layer was separated and the aqueous layer was extracted with DCM (5 mL×2). The combined organic layers were washed with NaHCO₃ (20 mL×2) and brine (20 mL), dried, concentrated and purified by prep-HPLC affording N-(5-((4-(6-cyano-1-methyl-1H-indazol-4-yl)pyrimidin-2-yl) amino)-2-((2-(dimethylamino)ethyl)(methyl)amino)-4-methoxyphenyl)acrylamide (16 mg, 3%). ¹H NMR (300 MHz, DMSO-d₆): δ 8.70 (s, 1H), 8.61-8.54 (m, 4H), 8.28 (s, 1H), 7.58 (d, J=5.1 Hz, 1H), 7.04 (s, 1H), 6.45-6.31 (m, 1H), 6.30-6.28 (m, 1H), 6.25-6.19 (m, 1H), 5.74-5.71 (m, 1H), 4.12 (s, 3H), 3.78 (s, 3H), 2.98-2.92 (m, 2H), 2.73 (s, 3H), 2.40-2.22 (m, 8H). ESI-MS (m/z): 526.2 (M+H)⁺.

### Example 10 (Comparative). N-(2-((2-(Dimethylamino)ethyl)(methyl)amino)-4-methoxy-5-((4-(1-methyl-1H-indazol-4-yl)pyrimidin-2-yl)amino)phenyl)acrylamide

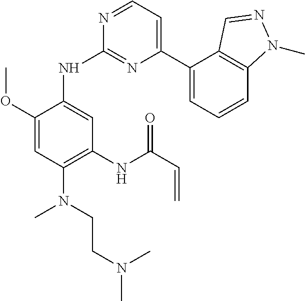 [SMILES: C=CC(=O)Nc1cc(Nc2nccc(-c3cccc4c3cnn4C)n2)c(OC)cc1N(C)CCN(C)C]

A solution of 2-chloro-4-(1-methyl-1H-indazol-4-yl)pyrimidine (300 mg, 1.22 mmol, 1.0 eq), N-(5-amino-2-((2-(dimethylamino)ethyl)(methyl)amino)-4-methoxy phenyl)acrylamide (393 mg, 1.34 mmol, 1.1 eq) and p-TsOH.H₂O (255 mg, 1.34 mmol, 1.1 eq) in 2-pentanol (12 mL) were heated at 150° C. in a microwave reactor for 1 h. After completion, the mixture was cooled to RT and diluted with MeOH/DCM=1:10 (20 mL) and sat. NaHCO₃ (5 mL). The organic layer was separated, washed with brine, dried over Na₂SO₄, filtered and concentrated to give a crude residue, which was purified by prep-HPLC to afford N-(2-((2-(dimethylamino)ethyl)(methyl)amino)-4-methoxy-5-((4-(1-methyl-1H-indazol-4-yl)pyrimidin-2-yl)amino)phenyl)acrylamide (78 mg, 12%). ¹H NMR (300 MHz, CDCl₃): δ 10.06 (br, 1H), 9.70 (s, 1H), 8.63 (d, J=5.1 Hz, 1H), 8.54 (s, 1H), 8.02 (d, J=5.7 Hz, 1H), 7.73 (s, 1H), 7.58-7.52 (m, 2H), 7.29 (m, 1H), 6.81 (s, 1H), 6.49-6.44 (m, 2H), 5.72-5.68 (m, 1H), 4.14 (s, 3H), 3.91 (s, 3H), 2.94-2.93 (m, 2H), 2.73 (s, 3H), 2.34-2.32 (s, 8H). ESI-MS (m/z): 501.3 (M+H)⁺. HPLC: 99.1%.

### Example 11. N-(5-((4-(1,3-Dimethyl-1H-pyrrolo[2,3-b]pyridin-5-yl)pyrimidin-2-yl)amino)-2-((2-(dimethylamino)ethyl)(methyl)amino)-4-methoxyphenyl) acrylamide

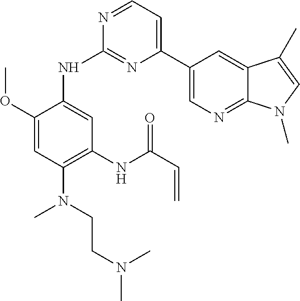 [SMILES: C=CC(=O)Nc1cc(Nc2nccc(-c3cnc4c(c3)c(C)cn4C)n2)c(OC)cc1N(C)CCN(C)C]

To a 10 mL microwave reactor were added 2-chloro-4-(1,3-dimethyl-1H-pyrrolo[2,3-b]pyridin-5-yl)pyrimidine (300 mg, 1.16 mmol, 1.0 eq), N-(5-amino-2-((2-(dimethylamino)ethyl)(methyl)amino)-4-methoxy phenyl)acrylamide (115 mg, 0.394 mmol, 1.1 eq), 2-pentanol (3 mL) and p-TsOH.H₂O (243 mg, 1.28 mmol, 1.1 eq). The mixture was stirred at 160° C. in a microwave for 40 min. After completion, the mixture was cooled to RT and diluted with sat. NaHCO₃ (10 mL) and DCM/MeOH (10/1, 20 mL), the organic layer was separated and the aqueous layer was extracted with DCM/MeOH (10/1, 2×5 mL). The combined organic layers were washed with NaHCO₃ (10 mL) and brine (10 mL), dried, concentrated and purified by prep-HPLC affording N-(5-((4-(1,3-dimethyl-1H-pyrrolo[2,3-b]pyridin-5-yl)pyrimidin-2-yl)amino)-2-((2-(dimethylamino)ethyl)(methyl)amino)-4-methoxyphenyl) acrylamide (63 mg, 11%). ¹H NMR (300 MHz, DMSO-d₆): δ 10.16 (br, 1H), 9.11-9.07 (m, 2H), 8.79 (s, 1H), 8.47 (s, 1H), 8.13 (s, 1H), 7.51 (s, 1H), 7.34 (s, 1H), 7.04 (s, 1H), 6.42-6.40 (m, 1H), 6.25-6.19 (m, 1H), 5.80-5.74 (m, 1H), 3.86 (s, 3H), 3.79 (s, 3H), 2.89-2.87 (m, 2H), 2.72 (s, 3H), 2.48-2.42 (m, 2H), 2.28 (s, 3H), 2.22 (s, 6H). ESI-MS (m/z): 515.3 (M+H)⁺.

### Example 12. N-(5-((4-(1,3-Dimethyl-1H-indol-5-yl)pyrimidin-2-yl)amino)-2-((2-(dimethylamino)ethyl)(methyl)amino)-4-methoxyphenyl)acrylamide

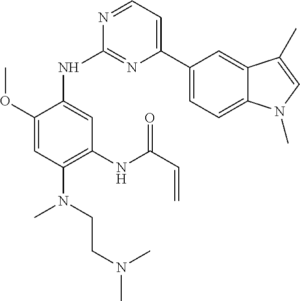 [SMILES: C=CC(=O)Nc1cc(Nc2nccc(-c3ccc4c(c3)c(C)cn4C)n2)c(OC)cc1N(C)CCN(C)C]

To a solution of 2-chloro-4-(1,3-dimethyl-1H-indol-5-yl)pyrimidine (300 mg, 1.16 mmol, 1.0 eq) in 2-pentanol (3 mL) were added N-(5-amino-2-((2-(dimethylamino)ethyl)(methyl)amino)-4-methoxy phenyl)acrylamide (375 mg, 1.28 mmol, 1.1 eq) and p-TsOH.H₂O (244 mg, 1.28 mmol, 1.1 eq). The mixture was stirred in a microwave at 160° C. for 40 minutes. After completion, the mixture was cooled to RT and diluted with sat. NaHCO₃ (10 mL) and DCM/MeOH (10/1, 20 mL), the organic layer was separated and the aqueous layer was extracted with DCM/MeOH (10/1, 2×5 mL). The combined organic layers were washed with NaHCO₃ (10 mL) and brine (10 mL), dried, concentrated and purified by prep-HPLC affording N-(5-((4-(1,3-dimethyl-1H-indol-5-yl)pyrimidin-2-yl)amino)-2-((2-(dimethylamino)ethyl)(methyl)amino)-4-methoxyphenyl)acrylamide (65 mg, 10.8%). ¹H NMR (300 MHz, DMSO-d₆): δ 10.14 (br, 1H), 9.11 (s, 1H), 8.41 (s, 2H), 8.08 (d, J=7.8 Hz, 1H), 7.99 (s, 1H), 7.45-7.43 (m, 2H), 7.13 (s, 1H), 7.02 (s, 1H), 6.46-6.37 (m, 1H), 6.28-6.22 (m, 1H), 5.77-5.74 (m, 1H), 3.87 (s, 3H), 3.75 (s, 3H), 2.90-2.88 (m, 2H), 2.71 (s, 3H), 2.32-2.30 (m, 2H), 2.28 (s, 3H), 2.22 (s, 6H). ESI-MS (m/z): 514.3 (M+H)⁺.

### Example 13 (Comparative). N-(5-((4-(3-Chloro-1-methyl-1H-pyrrolo[2,3-b]pyridin-5-yl) pyrimidin-2-yl)amino)-2-((2-(dimethylamino)ethyl)(methyl)amino)-4-methoxyphenyl)acrylamide

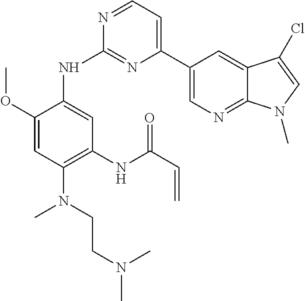 [SMILES: C=CC(=O)Nc1cc(Nc2nccc(-c3cnc4c(c3)c(Cl)cn4C)n2)c(OC)cc1N(C)CCN(C)C]

To a 10 mL microwave reactor were added 2-chloro-4-(3-chloro-1-methyl-1H-pyrrolo[2,3-b]pyridin-5-yl)pyrimidine (100 mg, 0.358 mmol, 1.0 eq), N-(5-amino-2-((2-(dimethylamino)ethyl)(methyl)amino)-4-methoxyphenyl)acrylamide (115 mg, 0.394 mmol, 1.1 eq), 2-pentanol (1 mL), and p-TsOH.H₂O (75 mg, 0.39 mmol, 1.1 eq). The mixture was stirred at 160° C. for 1 h in a microwave. After completion, the mixture was cooled to RT and diluted with sat. NaHCO₃ (10 mL), and extracted with DCM/MeOH (10/1, 10 mL, 5 mL 5 mL). The combined organic layers were washed with NaHCO₃ (5 mL) and brine (5 mL), dried, concentrated and purified by prep-HPLC affording N-(5-((4-(3-chloro-1-methyl-1H-pyrrolo[2,3-b]pyridin-5-yl)pyrimidin-2-yl)amino)-2-((2-(dimethylamino)ethyl)(methyl)amino)-4-methoxyphenyl)acrylamide (8 mg, 5%). ¹HNMR (300 MHz, DMSO-d₆): δ 10.13 (br, 1H), 9.16 (s, 1H), 8.97 (s, 1H), 8.73 (s, 1H), 8.49 (s, 1H), 8.26 (s, 1H), 7.82 (s, 1H), 7.56 (s, 1H), 7.02 (s, 1H), 6.39-6.36 (m, 1H), 6.33-6.20 (m, 1H), 5.76-5.74 (m, 1H), 3.85 (s, 6H), 2.88-2.86 (m, 2H), 2.72 (s, 3H), 2.31-2.30 (m, 2H), 2.21 (s, 6H). ESI-MS (m/z): 535.2 (M+H)⁺.

### Example 14. N-(2-((2-(Dimethylamino)ethyl)(methyl)amino)-5-((4-(3-(2-hydroxy acetyl)-1H-indol-1-yl)pyrimidin-2-yl)amino)-4-methoxyphenyl)acrylamide

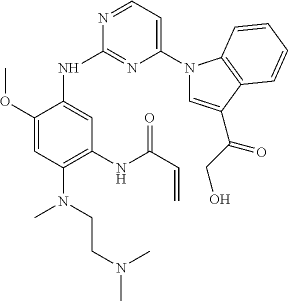 [SMILES: C=CC(=O)Nc1cc(Nc2nccc(-n3cc(C(=O)CO)c4ccccc43)n2)c(OC)cc1N(C)CCN(C)C]

To a solution of 2-(1-(2-chloropyrimidin-4-yl)-1H-indol-3-yl)-2-oxoethyl acetate (165 mg, 0.5 mmol, 1.0 eq) and N-(5-amino-2-((2-(dimethylamino)ethyl)(methyl) amino)-4-methoxyphenyl)acrylamide (160 mg, 0.55 mmol, 1.1 eq) in 2-pentanol (3 mL) was added p-TsOH.H₂O (105 mg, 0.55 mmol, 1.1 eq) over a period of 10 min. The mixture was heated to 100° C. for 2 h. The mixture was poured into water (10 mL), then adjusted to pH=7 with saturated sodium bicarbonate solution, extracted with EA (10 mL×2), dried over sodium sulfate, concentrated to afford the desired diarylamine (30 mg, 10%). LCMS: (M+H)⁺: 585.8.

To a solution of the above diarylamine (30 mg, 0.05 mmol, 1.0 eq) in MeOH (3 mL) was added K₂CO₃ (20 mg, 0.15 mmol, 3.0 eq). The reaction was stirred at room temperature for 1 hour. After completion, the mixture was filtered; the filtrate was concentrated in vacuo and purified by silica column chromatography affording the desired product N-(2-((2-(dimethylamino)ethyl)(methyl)amino)-5-((4-(3-(2-hydroxy acetyl)-1H-indol-1-yl)pyrimidin-2-yl)amino)-4-methoxyphenyl)acrylamide (2 mg, 7%). ¹H NMR (300 MHz, DMSO-d₆): δ 9.40 (s, 1H), 9.12 (s, 1H), 8.72-8.54 (m, 2H), 8.25-8.24 (m, 2H), 7.28-7.26 (m, 2H), 7.05-7.04 (m, 1H), 6.74-6.72 (m, 1H), 6.22-6.18 (m, 1H), 5.73-5.69 (m, 1H), 5.32 (br, 1H), 5.09 (s, 1H), 4.63 (s, 2H), 3.84 (s, 3H), 3.33-3.31 (m, 2H), 2.68 (s, 3H), 2.66-2.64 (m, 2H), 2.50 (s, 6H). ESI-MS (m/z): 543.8 (M+H)⁺. HPLC: 75.1%.

### Example 15. N-(2-((2-(Dimethylamino)ethyl)(methyl)amino)-4-methoxy-5-((4-(3-(methylsulfonamido)-1H-indazol-1-yl)pyrimidin-2-yl)amino)phenyl)acrylamide

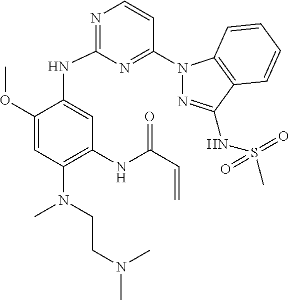 [SMILES: C=CC(=O)Nc1cc(Nc2nccc(-n3nc(NS(C)(=O)=O)c4ccccc43)n2)c(OC)cc1N(C)CCN(C)C]

To a 100 mL four-neck flask were added N-(1-(2-chloropyrimidin-4-yl)-1H-indazol-3-yl)methanesulfonamide (290 mg, 0.89 mmol, 1.0 eq), N-(5-amino-2-((2-(dimethylamino)ethyl)(methyl) amino)-4-methoxyphenyl)acrylamide (284 mg, 0.98 mmol, 1.1 eq), 2-pentanol (5 mL) and p-TsOH.H₂O (185 mg, 0.97 mmol, 1.1 eq). The mixture was stirred at 120° C. for 2 h. After completion, the mixture was cooled to RT and diluted with water (10 mL) and DCM/MeOH (10/1, 20 mL), the organic layer was separated and the aqueous layer was extracted with DCM (5 mL×2). The combined organic layers were washed with NaHCO₃ (20 mL×2) and brine (20 mL), the combined organic layers were dried, concentrated and purified by prep-HPLC affording N-(2-((2-(dimethylamino)ethyl)(methyl)amino)-4-methoxy-5-((4-(3-(methylsulfonamido)-1H-indazol-1-yl)pyrimidin-2-yl)amino)phenyl)acrylamide (20 mg, 4%). ¹H NMR (300 MHz, DMSO-d₆): δ 9.96 (br, 1H), 8.67 (s, 1H), 8.42-8.34 (m, 3H), 7.87 (d, J=7.5 Hz, 1H), 7.36-7.33 (m, 1H), 7.28-7.24 (m, 1H), 7.12-7.04 (m, 2H), 6.52-6.44 (m, 1H), 6.22-6.17 (m, 1H), 5.73 (d, J=10.8 Hz, 1H), 3.77 (s, 3H), 3.32 (s, 3H), 3.03-3.00 (m, 2H), 2.73 (s, 3H), 2.61-2.58 (m, 2H), 2.38 (s, 6H). ESI-MS (m/z): 579.7 (M−H)⁻. HPLC: 85.6%.

### Example 16. N-(2-((2-(Dimethylamino)ethyl)(methyl)amino)-4-methoxy-5-((4-(1-(methylamino)imidazo[1,5-a]pyridin-3-yl)pyrimidin-2-yl)amino)phenyl) acrylamide

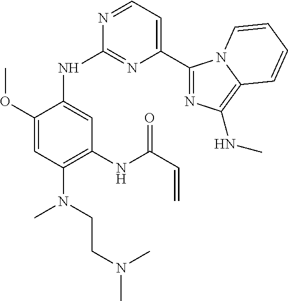 [SMILES: C=CC(=O)Nc1cc(Nc2nccc(-c3nc(NC)c4ccccn34)n2)c(OC)cc1N(C)CCN(C)C]

To a solution of 2-chloro-4-(1-(N-methylamino)imidazo[1,5-a]pyridin-3-yl)pyrimidine (128 mg, 0.49 mmol, 1.0 eq) and N-(5-amino-2-((2-(dimethylamino)ethyl)(methyl) amino)-4-methoxyphenyl)acrylamide (137 mg, 0.49 mmol, 1.0 eq) in 2-pentanol (4 mL) was added PTSA (103 mg, 0.53 mmol, 1.1 eq). The reaction was stirred at 100° C. for 2 hours. After cooling down to RT, the mixture was diluted with water (50 mL), extracted with DCM (50 mL×3, washed with brine (50 mL), concentrated and the residue was purified by prep-HPLC affording N-(2-((2-(dimethylamino) ethyl)(methyl)amino)-4-methoxy-5-((4-(1-(methylamino)imidazo[1,5-a]pyridin-3-yl)pyrimidin-2-yl)amino)phenyl) acrylamide (12 mg, 5%). ESI-MS (m/z): 515.9 (M+H)⁺. HPLC: 66.1%.

### Example 17. N-(2-((2-(Dimethylamino)ethyl)(methyl)amino)-4-methoxy-5-((4-(3-(methoxymethyl)-1H-indol-1-yl)pyrimidin-2-yl)amino)phenyl)acrylamide

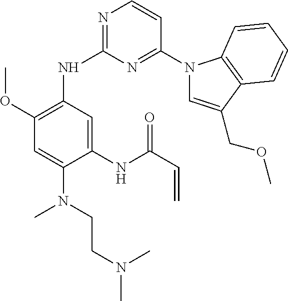 [SMILES: C=CC(=O)Nc1cc(Nc2nccc(-n3cc(COC)c4ccccc43)n2)c(OC)cc1N(C)CCN(C)C]

### Methyl 1-(2-((4-fluoro-2-methoxy-5-nitrophenyl)amino)pyrimidin-4-yl)-1H-indole-3-carboxylate

To a 250 mL four-neck flask were added methyl 1-(2-chloropyrimidin-4-yl)-1H-indole-3-carboxylate (6.0 g, 20.9 mmol, 1.0 eq), 4-fluoro-2-methoxy-5-nitroaniline (4.6 g, 24.7 mmol, 1.2 eq), 2-pentanol (110 mL) and p-TsOHH₂O (5.4 g, 28.4 mmol, 1.4 eq). The mixture was refluxed for 2 h. After completion, the precipitate was collected by filtration and the solid was re-dissolved in water (30 mL) then adjusted to pH=8-9 with aqueous ammonia. The solid was filtered, washed with water (100 mL×2) and dried to give methyl 1-(2-((4-fluoro-2-methoxy-5-nitrophenyl)amino)pyrimidin-4-yl)-1H-indole-3-carboxylate (6.3 g, 77%).

¹HNMR (300 MHz, DMSO-d₆) δ 9.14 (s, 1H), 8.82 (s, 1H), 8.68 (d, J=8.4 Hz, 1H), 8.58-8.56 (m, 2H), 8.11 (d, J=7.8 Hz, 1H), 7.51-7.28 (m, 4H), 3.98 (s, 3H), 3.88 (s, 3H).

### Methyl 1-(2-((4-((2-(dimethylamino)ethyl)(methyl)amino)-2-methoxy-5-nitrophenyl)amino)pyrimidin-4-yl)-1H-indole-3-carboxylate

A 250 mL sealed tube was charged with methyl 1-(2-((4-fluoro-2-methoxy-5-nitrophenyl)amino) pyrimidin-4-yl)-1H-indole-3-carboxylate (6.3 g, 7.6 mmol, 1.0 eq), DIPEA (5.9 g, 45 mmol, 6.0 eq), DMAc (70 mL) and N,N,N′-trimethylethane-1,2-diamine (2.35 g, 22.9 mmol, 3.0 eq). The mixture was heated to 120° C. and monitored by TLC and LCMS. After completion, the mixture was poured into water (400 mL) and extracted with EA (3×200 mL). The combined organic layer was washed with brine (3×200 mL), dried over Na₂SO₄, concentrated and purified by column chromatography to give the crude product methyl 1-(2-((4-((2-(dimethylamino)ethyl)(methyl)amino)-2-methoxy-5-nitrophenyl)amino) pyrimidin-4-yl)-1H-indole-3-carboxylate (12.0 g, 56%).

### (1-(2-((4-((2-(Dimethylamino)ethyl)(methyl)amino)-2-methoxy-5-nitrophenyl) amino)pyrimidin-4-yl)-1H-indol-3-yl)methanol

A 500 mL four-neck flask was charged with 1-(2-((4-((2-(dimethylamino)ethyl) (methyl)amino)-2-methoxy-5-nitrophenyl)amino)pyrimidin-4-yl)-1H-indole-3-carboxylate (12.0 g, 23.1 mmol, 1.0 eq) and THF (100 mL). The mixture was cooled to −78° C. and DIBAL-H (104 mL, 92.4 mmol, 4.0 eq) was added dropwise. After addition, the mixture was stirred at this temperature for 30 minutes and then warmed to −40° C. The reaction was monitored by TLC. After completion, the reaction was quenched at −40° C. by cautious batchwise addition of Na₂SO₄.10H₂O (Glauber's salt 50 g) with vigorous stirring. After the quench was completed, the reaction mixture was slowly warmed to 0° C. When gas evolution had ceased, and the precipitate had granulated, the slurry was vacuum filtered, and the residue rinsed with DCM (2×100 mL). The combined filtrates was dried, concentrated and purified by column chromatography to give the product (1-(2-((4-((2-(dimethylamino)ethyl)(methyl)amino)-2-methoxy-5-nitrophenyl)amino)pyrimidin-4-yl)-1H-indol-3-yl)methanol (6.0 g, 53%). ¹H NMR (300 MHz, CDCl₃) δ 9.16 (s, 1H), 8.43 (d, J=5.7 Hz, 1H), 8.17 (d, J=8.7 Hz, 1H), 7.97 (s, 1H), 7.74 (d, J=7.5 Hz, 1H), 7.54 (s, 1H), 7.37-7.27 (m, 3H), 6.94 (d, J=5.7 Hz, 1H), 6.66 (s, 1H), 4.96 (s, 2H), 3.97 (s, 3H), 3.31 (t, J=7.2 Hz, 2H), 2.89 (s, 3H), 2.62 (t, J=7.2 Hz, 2H), 2.31 (s, 6H).

### N¹-(2-(Dimethylamino)ethyl)-5-methoxy-N⁴-(4-(3-(methoxymethyl)-1H-indol-1-yl)pyrimidin-2-yl)-N¹-methyl-2-nitrobenzene-1,4-diamine

To a 100 mL three-neck flask were added (1-(2-((4-((2-(dimethylamino)ethyl)(methyl)amino)-2-methoxy-5-nitrophenyl) amino)pyrimidin-4-yl)-1H-indol-3-yl)methanol (3.2 g, 6.5 mmol, 1.0 eq) and DMF (25 mL). The mixture was cooled to −40° C. and NaH (172 mg, 7.1 mmol, 1.1 eq) was added portion wise. After stirring at this temperature for 30 minutes, MeI (500 mg, 11.3 mmol, 1.7 eq) was added. The mixture was stirred for another 30 minutes and TLC showed reaction completion. The reaction mixture was poured into water (100 mL), and extracted with EA (100 mL×2). The combined organic layer was dried, concentrated and purified by column chromatography to give the product N¹-(2-(dimethylamino)ethyl)-5-methoxy-N⁴-(4-(3-(methoxymethyl)-1H-indol-1-yl)pyrimidin-2-yl)-N¹-methyl-2-nitrobenzene-1,4-diamine (700 mg, 22%). ¹H NMR (300 MHz, CDCl₃) 8.29 (s, 1H), 7.90 (s, 1H), 7.72-7.54 (m, 3H), 7.18-7.10 (m, 1H), 6.78 (s, 1H), 6.62 (d, J=5.4 Hz, 1H), 4.82 (s, 2H), 3.80 (s, 3H), 3.61-3.58 (m, 2H), 3.43 (s, 3H), 3.10-3.02 (m, 2H), 2.92 (s, 3H), 2.57 (s, 6H).

### N¹-(2-(Dimethylamino)ethyl)-5-methoxy-N⁴-(4-(3-(methoxymethyl)-1H-indol-1-yl)pyrimidin-2-yl)-N¹-methylbenzene-1,2,4-triamine

To a 100 mL three-neck flask were added N¹-(2-(dimethylamino)ethyl)-5-methoxy-N⁴-(4-(3-(methoxymethyl)-1H-indol-1-yl)pyrimidin-2-yl)-N¹-methyl-2-nitrobenzene-1,4-diamine (700 mg, 1.3 mmol), 10% Pd/C (500 mg) and MeOH (5 mL). The mixture was stirred in a H₂ atmosphere for 2 hours. After completion, the mixture was filtered and washed with MeOH. The filtrate was concentrated to give the product N¹-(2-(dimethylamino)ethyl)-5-methoxy-N⁴-(4-(3-(methoxymethyl)-1H-indol-1-yl) pyrimidin-2-yl)-N¹-methylbenzene-1,2,4-triamine (390 mg, crude) which was used in the next step without further purification. ESI-MS (m/z): 476 (M+H)⁺.

### N-(2-((2-(Dimethylamino)ethyl)(methyl)amino)-4-methoxy-5-((4-(3-(methoxymethyl)-1H-indol-1-yl)pyrimidin-2-yl)amino)phenyl)acrylamide

To a solution of N¹-(2-(dimethylamino)ethyl)-5-methoxy-N⁴-(4-(3-(methoxymethyl)-1H-indol-1-yl)pyrimidin-2-yl)-N¹-methylbenzene-1,2,4-triamine (390 mg, 0.82 mmol) in DCM (4 mL) was added acryloyl chloride (38 mg, 0.5 mmol) at −5 to 0° C. The mixture was stirred at RT for 1 h. After completion, the mixture was diluted with DCM (10 mL), washed with saturated NaHCO₃ (10 mL) and brine (10 mL). The organic layer was dried, concentrated and purified by prep-HPLC to give N-(2-((2-(dimethylamino)ethyl)(methyl)amino)-4-methoxy-5-((4-(3-(methoxymethyl)-1H-indol-1-yl)pyrimidin-2-yl)amino)phenyl)acrylamide (19 mg, 3% for 2 steps). ¹H NMR (300 MHz, DMSO-d₆) 10.11 (s, 1H), 8.46-8.41 (m, 1H), 8.27-8.22 (m, 1H), 7.93 (s, 1H), 7.74-7.71 (m, 1H), 7.57-7.52 (m, 1H), 7.23-7.01 (m, 3H), 6.80 (br, 1H), 6.45-6.36 (m, 1H), 6.21-6.15 (m, 1H), 5.74-5.71 (m, 1H), 4.63 (s, 2H), 3.69 (s, 3H), 3.34 (s, 3H), 2.94-2.90 (m, 2H), 2.73 (s, 3H), 2.42-2.39 (m, 2H), 2.23 (s, 6H). ESI-MS (m/z): 530.3 (M+H)⁺.

### Example 18. N-(4-(Difluoromethoxy)-5-((4-(3-(dimethylamino)-1H-indazol-1-yl)pyrimidin-2-yl)amino)-2-((2-(dimethylamino)ethyl)(methyl)amino)phenyl) acrylamide

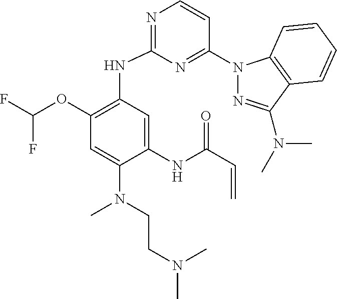 [SMILES: C=CC(=O)Nc1cc(Nc2nccc(-n3nc(N(C)C)c4ccccc43)n2)c(OC(F)F)cc1N(C)CCN(C)C]

To a 10 mL Schlenk tube were added N-(5-amino-4-(difluoromethoxy)-2-((2-(dimethylamino)ethyl)(methyl) amino)phenyl)acrylamide (151 mg, 0.55 mmol, 1.0 eq), 1-(2-chloropyrimidin-4-yl)-3-(N,N-dimethylamino)-1H-indazole (200 mg, 0.61 mmol, 1.1 eq), 2-pentanol (3 mL) and p-TsOH.H₂O (116 mg, 0.61 mmol, 1.1 eq). The mixture was stirred at 120° C. for 2 h. After completion, the mixture was cooled to RT and diluted with sat. NaHCO₃ (10 mL) and DCM/MeOH (10/1, 20 mL), the organic layer was separated and the aqueous layer was extracted with DCM (5 mL×2). The combined organic layers were washed with NaHCO₃ (20 mL×2) and brine (20 mL), dried, (Na₂SO₄) concentrated and purified by prep-HPLC affording N-(4-(difluoromethoxy)-5-((4-(3-(dimethylamino)-1H-indazol-1-yl)pyrimidin-2-yl)amino)-2-((2-(dimethylamino) ethyl)(methyl)amino)phenyl)acrylamide (38 mg, 12%). ¹H NMR (300 MHz, DMSO-d₆): δ 10.16 (br, 1H), 8.97 (s, 1H), 8.53-8.49 (m, 2H), 8.30 (d, J=4.8 Hz, 1H), 7.97 (d, J=7.5 Hz, 1H), 7.33-7.04 (m, 5H), 6.56 (m, 1H), 6.23-6.18 (m, 1H), 5.77-5.74 (m, 1H), 3.16 (s, 6H), 2.98-2.95 (m, 2H), 2.73 (s, 3H), 2.50-2.48 (m, 2H), 2.31 (s, 6H). ESI-MS (m/z): 566.2 (M+H)⁺. HPLC: 97.8%.

### Example 19. N-(4-(Difluoromethoxy)-5-((4-(3-(dimethylamino)-1H-pyrazolo[4,3-b]pyridin-1-yl)pyrimidin-2-yl)amino)-2-((2-(dimethylamino)ethyl)(methyl)amino) phenylacrylamide

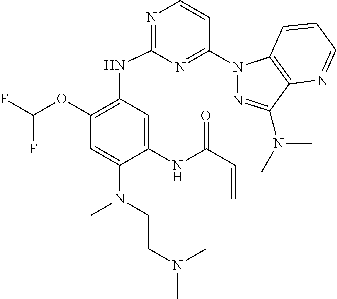 [SMILES: C=CC(=O)Nc1cc(Nc2nccc(-n3nc(N(C)C)c4ncccc43)n2)c(OC(F)F)cc1N(C)CCN(C)C]

To a 10 mL Schlenk tube were added 2-chloro-4-(3-(N,N-dimethylamino)-1H-pyrazolo[4,3-b]pyridin-1-yl)pyrimidine (274 mg, 1.0 mmol, 1 eq), N-(5-amino-4-(difluoromethoxy)-2-((2-(dimethylamino)ethyl)(methyl)amino)phenyl)acrylamide (360 mg, 1.1 mmol, 1.1 eq), 2-pentanol (5 mL) and p-TsOH.H₂O (116 mg, 0.61 mmol, 0.61 eq). The mixture was stirred at 120° C. for 2 h. After completion, the mixture was cooled to RT and diluted with sat. NaHCO₃ (10 mL) and DCM/MeOH (10/1, 20 mL), the organic layer was separated and the aqueous layer was extracted with DCM (5 mL×2). The combined organic layers were washed with NaHCO₃ (20 mL×2) and brine (20 mL), dried, (Na₂SO₄) concentrated and purified by prep-HPLC affording N-(4-(difluoromethoxy)-5-((4-(3-(dimethylamino)-1H-pyrazolo[4,3-b]pyridin-1-yl) pyrimidin-2-yl)amino)-2-((2-(dimethylamino)ethyl)(methyl)amino)phenylacrylamide (55 mg, 9.7%). ¹H NMR (300 MHz, DMSO-d₆): δ 10.24 (br, 1H), 9.06 (s, 1H), 8.75 (br, 1H), 8.57-8.51 (m, 2H), 8.33 (d, J=4.8 Hz, 1H), 7.33-7.05 (m, 4H), 6.46-6.38 (m, 1H), 6.23-6.17 (m, 1H), 5.78-5.75 (m, 1H), 3.42 (s, 6H), 2.89 (m, 2H), 2.74 (s, 3H), 2.39 (m, 2H), 2.22 (s, 6H). ESI-MS (m/z): 567.2 (M+H)⁺. HPLC: 95.0%.

### Example 20. N-(4-(Difluoromethoxy)-2-((2-(dimethylamino)ethyl)(methyl) amino)-5-((4-(3-(ethylamino)-1H-pyrazolo[4,3-b]pyridin-1-yl)pyrimidin-2-yl)amino) phenyl)acrylamide

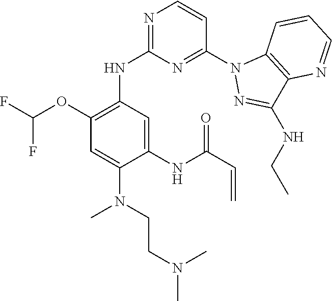 [SMILES: C=CC(=O)Nc1cc(Nc2nccc(-n3nc(NCC)c4ncccc43)n2)c(OC(F)F)cc1N(C)CCN(C)C]

To a 10 mL Schlenk tube were added 2-chloro-4-(3-(N-ethylamino)pyrazolo[4,3-b]pyrid-1-yl)pyrimidine (274 mg, 1.0 mmol, 1.0 eq), N-(5-amino-4-(difluoromethoxy)-2-((2-(dimethylamino)ethyl)(methyl)amino)phenyl)acrylamide (360 mg, 1.1 mmol, 1.1 eq), 2-pentanol (5 mL) and p-TsOH.H₂O (116 mg, 0.61 mmol, 0.61 eq). The mixture was stirred at 120° C. for 2 h. After completion, the mixture was cooled to RT and diluted with sat. NaHCO₃ (10 mL) and DCM/MeOH (10/1, 20 mL), the organic layer was separated and the aqueous layer was extracted with DCM (5 mL×2). The combined organic layers were washed with NaHCO₃ (20 mL×2) and brine (20 mL), dried, concentrated and purified by prep-HPLC affording the desired product N-(4-(difluoromethoxy)-2-((2-(dimethylamino)ethyl)(methyl)amino)-5-((4-(3-(ethyl amino)-1H-pyrazolo[4,3-b]pyridin-1-yl)pyrimidin-2-yl)amino)phenyl)acrylamide (2 mg, 0.3%). ¹H NMR (300 MHz, DMSO-d₆): δ 10.23 (br, 1H), 8.98 (s, 1H), 8.66-8.60 (m, 1H), 8.58 (s, 1H), 8.49 (d, J=4.8 Hz, 1H), 8.30 (d, J=4.8 Hz, 1H), 7.31-7.30 (m, 1H), 7.20 (s, 1H), 7.05 (s, 1H), 6.94-6.90 (m, 1H), 6.42-6.37 (m, 1H), 6.23-6.17 (m, 1H), 5.78-5.75 (m, 1H), 3.43-3.42 (m, 2H), 2.90-2.88 (m, 2H), 2.74 (s, 3H), 2.39-2.38 (m, 2H), 2.23 (s, 6H), 1.26-1.24 (m, 3H). ESI-MS (m/z): 567.2 (M+H)⁺.

### Example 21. N-(4-(2,2-Difluoroethoxy)-5-((4-(3-(dimethylamino)-1H-pyrazolo[4,3-b]pyridin-1-yl)pyrimidin-2-yl)amino)-2-(N-(2-(dimethylamino)ethyl)-N-methylamino)phenyl)acrylamide

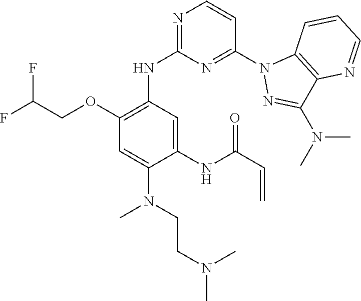 [SMILES: C=CC(=O)Nc1cc(Nc2nccc(-n3nc(N(C)C)c4ncccc43)n2)c(OCC(F)F)cc1N(C)CCN(C)C]

A solution of N-(5-amino-4-(2,2-difluoroethoxy)-2-((2-(dimethylamino)ethyl) (methyl)amino)phenyl)acrylamide (80 mg, 0.23 mmol, 1.0 eq), 2-chloro-4-(3-(N,N-dimethylamino)-1H-pyrazolo[4,3-b]pyridin-1-yl) pyrimidine (64 mg, 0.23 mmol, 1.0 eq) and p-TsOH.H₂O (43 mg, 0.25 mmol, 1.1 eq) in 2-pentanol (2 mL) was heated at 140° C. in a microwave for 30 min. After completion, the mixture was cooled to RT and diluted with sat. NaHCO₃ (10 mL) and DCM/MeOH (10/1, 20 mL), the organic layer was separated and the aqueous layer was extracted with DCM (5 mL×2). The combined organic layers were washed with NaHCO₃ (20 mL×2) and brine (20 mL), dried, concentrated and purified by prep-HPLC affording N-(4-(2,2-difluoroethoxy)-5-((4-(3-(dimethylamino)-1H-pyrazolo[4,3-b]pyridin-1-yl)pyrimidin-2-yl)amino)-2-(N-(2-(dimethylamino)ethyl)-N-methylamino)phenyl)acrylamide (8 mg, 4.7%). ¹H NMR (300 MHz, DMSO-d₆): δ 10.14 (br, 1H), 8.69-8.62 (m, 2H), 8.58-8.52 (m, 2H), 8.34-8.30 (m, 1H), 7.28-7.09 (m, 3H), 6.43-6.27 (m, 1H), 6.24-6.13 (m, 2H), 5.76-5.72 (m, 1H), 4.30-4.20 (m, 2H), 3.32 (s, 6H), 3.03-3.01 (m, 2H), 2.92 (s, 3H), 2.40-2.38 (m, 2H), 2.25 (s, 6H). ESI-MS (m/z): 581.3 (M+H)⁺. HPLC: 98.0%.

### Example 22. N-(4-(2,2-Difluoroethoxy)-2-((2-(dimethylamino)ethyl)(methyl) amino)-5-((4-(3-(ethylamino)-1H-pyrazolo[4,3-b]pyridin-1-yl)pyrimidin-2-yl) amino)phenyl)acrylamide

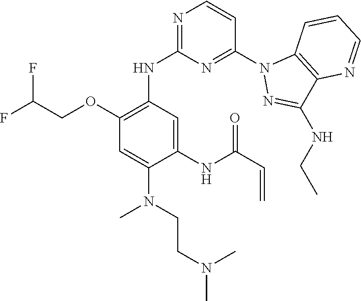 [SMILES: C=CC(=O)Nc1cc(Nc2nccc(-n3nc(NCC)c4ncccc43)n2)c(OCC(F)F)cc1N(C)CCN(C)C]

A solution of N-(5-amino-4-(2,2-difluoroethoxy)-2-((2-(dimethylamino)ethyl) (methyl)amino)phenyl)acrylamide (264 mg, 0.77 mmol, 1.1 eq), 2-chloro-4-(3-(N-ethylamino)pyrazolo[4,3-b]pyrid-1-yl)pyrimidine (212 mg, 0.77 mmol, 1.0 eq) and p-TsOH.H₂O (144 mg, 0.85 mmol, 1.1 eq) in 2-pentanol (2 mL) was heated to 140° C. in a microwave for 30 min. After completion, the mixture was cooled to RT and diluted with sat. NaHCO₃ (10 mL) and DCM/MeOH (10:1, 20 mL), the organic layer was separated and the aqueous layer was extracted with DCM (5 mL×2). The combined organic layers were washed with NaHCO₃ (10 mL×2) and brine (10 mL), dried, concentrated and purified by prep-HPLC affording N-(4-(2,2-difluoroethoxy)-2-((2-(dimethylamino)ethyl)(methyl)amino)-5-((4-(3-(ethylamino)-1H-pyrazolo[4,3-b]pyridin-1-yl)pyrimidin-2-yl)amino)phenyl)acrylamide (38 mg, 8.5%). ¹H NMR (300 MHz, DMSO-d₆): δ 10.16 (br, 1H), 8.62-8.41 (m, 3H), 8.40 (s, 1H), 8.32 (s, 1H), 7.29-7.27 (m, 1H), 7.22 (s, 1H), 7.16-7.11 (m, 1H), 6.98-6.94 (m, 1H), 6.47-6.13 (m, 3H), 5.74-5.70 (m, 1H), 4.33-4.25 (m, 2H), 3.38-3.34 (m, 2H), 2.91-2.90 (m, 2H), 2.75 (s, 3H), 2.37-2.35 (m, 2H), 2.23 (s, 6H), 1.26 (t, J=3.6 Hz, 3H). ESI-MS (m/z): 581.3 (M+H)⁺.

### Example 23. N-(2-((2-(dimethylamino)ethyl)(methyl)amino)-4-methoxy-5-((4-(3-(methylamino)-1H-thieno[2,3-c]pyrazol-1-yl)pyrimidin-2-yl)amino)phenyl)acrylamide

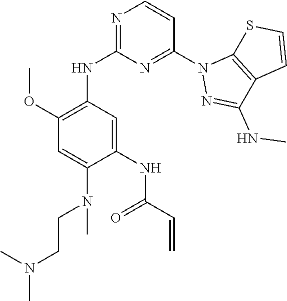 [SMILES: C=CC(=O)Nc1cc(Nc2nccc(-n3nc(NC)c4ccsc43)n2)c(OC)cc1N(C)CCN(C)C]

To a 10 mL Schlenk tube were added 2-chloro-4-(5-chloro-3-(methylamino)-1H-thieno[2,3-c]pyrazol-1-yl)pyrimidine (50 mg, 0.189 mmol, 1.0 eq), N-(5-amino-2-((2-(dimethylamino)ethyl)(methyl)amino)-4-methoxyphenyl)acrylamide (60 mg, 0.207 mmol, 1.1 eq) and 2-pentanol (1 mL) and p-TsOH.H₂O (40 mg, 0.207 mmol, 1.1 eq). The mixture was stirred at 120° C. for 2 h. After completion, the mixture was cooled to RT and diluted with water (3 mL) and DCM/MeOH (10/1, 4 mL), the organic layer was separated and the aqueous layer was extracted with DCM (5 mL×2). The combined organic layers were washed with NaHCO₃ (5 mL×2) and brine (5 mL), the combined organic layers were dried, concentrated and purified by prep-HPLC affording N-(2-((2-(dimethylamino) ethyl)(methyl)amino)-4-methoxy-5-((4-(3-(methylamino)-1H-thieno[2,3-c]pyrazol-1-yl)pyrimidin-2-yl)amino)phenyl)acrylamide (17 mg, 17%). ¹H NMR (300 MHz, CDCl₃): δ 10.12 (br, 1H), 9.38 (s, 1H), 8.42 (d, J=5.7 Hz, 1H), 7.38 (s, 1H), 7.12 (d, J=5.4 Hz, 1H), 6.94-6.89 (m, 2H), 6.80 (s, 1H), 6.39-6.36 (m, 2H), 5.68 (t, J=5.7 Hz, 1H), 4.21 (br, 1H), 3.90 (s, 3H), 3.10 (d, J=4.2 Hz, 3H), 2.95-2.92 (m, 2H), 2.74 (s, 3H), 2.35-2.33 (m, 8H). ESI-MS (m/z): 522.2 (M+H)⁺.

### Example 24. N-(5-((4-(5-chloro-3-(methylamino)-1H-thieno[2,3-c]pyrazol-1-yl) pyrimidin-2-yl)amino)-2-((2-(dimethylamino)ethyl)(methyl)amino)-4-methoxyphenyl)acrylamide

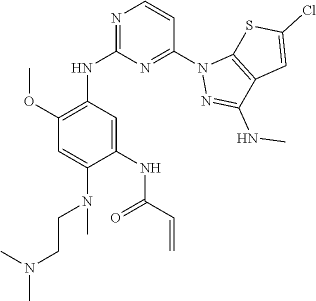 [SMILES: C=CC(=O)Nc1cc(Nc2nccc(-n3nc(NC)c4cc(Cl)sc43)n2)c(OC)cc1N(C)CCN(C)C]

To a solution of 2-chloro-4-(5-chloro-3-(methylamino)-1H-thieno[2,3-c]pyrazol-1-yl)pyrimidine (75 mg, 0.25 mmol, 1 eq) and N-(5-amino-2-((2-(dimethylamino)ethyl)(methyl)amino)-4-methoxyphenyl)acrylamide (80.4 mg, 0.275 mmol, 1.1 eq) in 2-pentanol (2 mL) was added p-TsOH.H₂O (52.3 mg, 0.275 mmol, 1.1 eq). The mixture was heated to 140° C. under microwave for 30 min. After completion, the mixture was cooled to RT and diluted with sat. NaHCO₃ (10 mL) and DCM/MeOH (10/1, 20 mL), the organic layer was separated and the aqueous layer was extracted with DCM (5 mL×2). The combined organic layers were washed with NaHCO₃ (20 mL×2) and brine (20 mL), dried, concentrated and purified by prep-HPLC affording N-(5-((4-(5-chloro-3-(methylamino)-1H-thieno[2,3-c]pyrazol-1-yl)pyrimidin-2-yl)amino)-2-((2-(dimethylamino)ethyl)(methyl)amino)-4-methoxyphenyl)acrylamide (20 mg, 14%). ¹H NMR (300 MHz, DMSO-d₆): δ10.19 (br, 1H), 8.64 (br, 1H), 8.38 (s, 1H), 8.31 (d, J=5.4 Hz, 1H), 7.08 (s, 1H), 7.05 (s, 1H), 6.85 (d, J=5.4 Hz, 1H), 6.69-6.68 (m, 1H), 6.42-6.33 (m, 1H), 6.21-6.15 (m, 1H), 5.74-5.70 (m, 1H), 3.72 (s, 3H), 2.93-2.90 (m, 2H), 2.86 (d, J=4.5 Hz, 3H), 2.75 (s, 3H), 2.36-2.32 (m, 2H), 2.22 (s, 6H)._ESI-MS (m/z): 556.2 (M+H)⁺

### Example 25 (Comparative). N-(2-((2-(dimethylamino)ethyl)(methyl)amino)-4-methoxy-5-((4-(1-methyl-1H-indol-4-yl)pyrimidin-2-yl)amino)phenyl)acrylamide

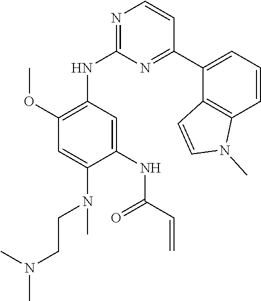 [SMILES: C=CC(=O)Nc1cc(Nc2nccc(-c3cccc4c3ccn4C)n2)c(OC)cc1N(C)CCN(C)C]

A solution of 2-chloro-4-(1-methyl-1H-indol-4-yl)pyrimidine (250 mg, 1.02 mmol, 1.0 eq), N-(5-amino-2-((2-(dimethylamino)ethyl)(methyl)amino)-4-methoxyphenyl)acrylamide (325 mg, 1.11 mmol, 1.1 eq) and p-TsOH.H₂O (215 mg, 1.13 mmol, 1.1 eq) in 2-pentanol (10 mL) was heated to 150° C. in a microwave for 1 h. After completion, the mixture was cooled to RT and diluted with MeOH:DCM=1:10 (20 mL) and sat. NaHCO₃ (5 mL). The organic layer was separated, washed with brine, concentrated and purified by prep-HPLC affording N-(2-((2-(dimethylamino) ethyl)(methyl)amino)-4-methoxy-5-((4-(1-methyl-1H-indol-4-yl)pyrimidin-2-yl)amino)phenyl)acrylamide (30 mg, 5.8%). ¹H NMR (300 MHz, CDCl₃): δ 10.01 (br, 1H), 9.68 (s, 1H), 8.58 (d, J=4.2 Hz, 1H), 7.87 (d, J=7.2 Hz, 1H), 7.70 (s, 1H), 7.44 (d, J=8.0 Hz, 1H), 7.37 (t, J=7.6 Hz, 1H), 7.25 (d, J=4.8 Hz, 1H), 7.16 (d, J=3.2 Hz, 1H), 7.00 (d, J=2.8 Hz, 1H), 6.77 (s, 1H), 6.48-6.36 (m, 2H), 5.68 (d, J=11.2 Hz, 1H), 3.87 (s, 3H), 3.84 (s, 3H), 2.95-2.90 (m, 2H), 2.70 (s, 3H), 2.28-2.20 (m, 8H). ESI-MS (m/z): 500.2 (M+H)⁺. HPLC: 96.8%.

### Example 26. N-(5-((4-(7-Cyano-1,3-dimethyl-1H-indol-5-yl)pyrimidin-2-yl)amino)-2-((2-(ethyl(methyl)amino)ethyl)(methyl)amino)-4-methoxyphenyl)acrylamide

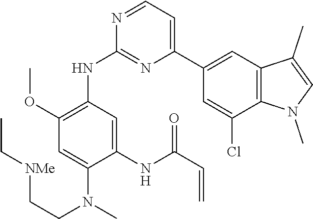 [SMILES: C=CC(=O)Nc1cc(Nc2nccc(-c3cc(Cl)c4c(c3)c(C)cn4C)n2)c(OC)cc1N(C)CCN(C)CC]

### N-Methyl-N-(2-(methylamino)ethyl)acetamide

To a solution of N¹,N²-dimethylethane-1,2-diamine (8.8 g, 100 mmol, 1.0 eq) in DCM (100 mL) was added TEA (20.2 g, 200 mmol, 2.0 eq), cooled to 0° C. and AcCl (7.8 g, 100 mmol) was added drop wise. The mixture was stirred at 0° C. for 2 h. Water (100 mL) was added to the reaction mixture, followed by extraction with DCM (3×100 mL). The combined organic layers were washed with brine and dried over MgSO₄, filtered and the filtrate was concentrated in vacuo to give a crude residue, which was used directly in the next without further purification (9.2 g, crude).

### N¹-Ethyl-N¹,N¹-dimethylethane-1,2-diamine

To a cooled solution of N-methyl-N-(2-(methylamino)ethyl)acetamide (6.5 g, 50 mmol, 1 eq) in THF (100 mL) under nitrogen was added LiAlH₄ (2.28 g, 60 mmol, 1.2 eq) carefully and the mixture was stirred at 0° C. for 30 minutes. Then the mixture was stirred at room temperature till completion. The reaction mixture was quenched with water, extracted with EA (150 mL×3). The combined organic layers were washed with brine, dried over sodium sulfate, filtered and the filtrate was concentrated in vacuo to give a residue, which was used directly in a subsequent reaction without further purification (3.8 g, crude). ESI-MS (m/z): 117.2 (M+H)⁺.

### 5-(2-((4-Fluoro-2-methoxy-5-nitrophenyl)amino)pyrimidin-4-yl)-1,3-dimethyl-1H-indole-7-carbonitrile

To a microwave reactor were added 2-chloro-4-(7-cyano-1,3-dimethyl-1H-indol-5-yl)pyrimidine (1.3 g, 4.6 mmol, 1.0 eq), 4-fluoro-2-methoxy-5-nitroaniline (0.86 g, 4.6 mmol, 1.0 eq), 2-pentanol (26 mL) and p-toluenesulfonic acid monohydrate (0.96 g, 5.06 mmol, 1.1 eq). The mixture was heated to 140° C. and stirred for 20 minutes. After cooling down to RT, the reaction was filtered and the filtrate was washed with CH₃CN (10 mL). The residue was then dispersed in CH₃CN (25 mL, refiltered, washed with CH₃CN (10 mL) and dried to give the desired product (1.6 g, 80%). ¹H NMR (300 MHz, DMSO-d₆): δ 9.25 (d, J=8.1 Hz, 1H), 8.71 (s, 1H), 8.59 (d, J=5.4 Hz, 1H), 8.54 (s, 1H), 8.47 (s, 1H), 7.72 (d, J=5.7 Hz, 1H), 7.47 (d, J=8.1 Hz, 1H), 7.11 (d, J=8.4 Hz, 1H), 4.05 (s, 3H), 4.03 (s, 3H), 2.34 (s, 3H). ESI-MS (m/z): 433.1 (M+H)⁺.

### 5-(2-((4-((2-(Ethyl(methyl)amino)ethyl) (methyl)amino)-2-methoxy-5-nitrophenyl)amino)pyrimidin-4-yl)-1,3-dimethyl-1H-indole-7-carbonitrile

To a 50 mL sealed tube were added 5-(2-((4-fluoro-2-methoxy-5-nitrophenyl)amino)pyrimidin-4-yl)-1,3-dimethyl-1H-indole-7-carbonitrile (0.5 g, 1.16 mmol, 1 eq), DIPEA (0.45 g, 3.47 mmol, 1.0 eq), DMAc (5 mL) and N¹-ethyl-N¹,N²-dimethylethane-1,2-diamine (202 mg, 1.74 mmol, 1.5 eq). The reaction was stirred at 120° C. till completion. After cooling down, the reaction was poured into water (20 mL) and extracted with EtOAc (20 mL×3). The combined organic layers were washed with water (20 mL×2) and brine (20 mL), dried, and concentrated under reduced pressure. The residue was purified by silica gel chromatography to give the desired product (320 mg, 52%). ¹HNMR (300 MHz, DMSO-d₆): δ 8.81 (s, 1H), 8.65 (s, 1H), 8.50 (d, J=4.8 Hz, 1H), 8.41 (s, 1H), 8.23 (s, 1H), 7.59 (d, J=4.8 Hz, 1H), 7.31 (s, 1H), 6.82 (s, 1H), 4.03 (s, 3H), 3.98 (s, 3H), 3.26 (t, J=6.0 Hz, 2H), 2.95-2.85 (m, 2H), 2.79 (s, 3H), 2.38-2.36 (m, 5H), 2.32 (s, 3H), 0.94 (t, J=7.2 Hz, 3H). ESI-MS (m/z): 529.2 (M+H)⁺.

### 5-(2-((5-Amino-4-((2-(ethyl(methyl)amino)ethyl) (methyl)amino)-2-methoxyphenyl)amino)pyrimidin-4-yl)-1,3-dimethyl-1H-indole-7-carbonitrile

To a solution of 5-(2-((4-((2-(ethyl(methyl)amino)ethyl)(methyl)amino)-2-methoxy-5-nitrophenyl)amino)pyrimidin-4-yl)-1,3-dimethyl-1H-indole-7-carbonitrile (320 mg, crude) in MeOH (5 mL) was added Pd/C (50 mg). The mixture was stirred under 1 atm hydrogen atmosphere for 1.5 hours. After completion, the reaction was filtered and washed with MeOH (5 mL×2). The filtrate was concentrated in vacuo to give the desired product (240 mg, crude) which was used in next step without purification.

¹H NMR (300 MHz, DMSO-d₆): δ 8.69 (s, 1H), 8.46-8.43 (m, 2H), 8.01 (s, 1H), 7.55-8.53 (m, 2H), 7.33 (s, 1H), 6.75 (s, 1H), 4.05 (s, 3H), 3.76 (s, 3H), 2.95 (s, 3H), 2.89-2.85 (m, 2H), 2.79 (s, 3H), 2.62-2.60 (m, 2H), 2.48-2.45 (m, 2H), 1.96 (s, 3H), 1.00 (t, J=6.8 Hz, 3H). ESI-MS (m/z): 499.2 (M+H)⁺.

### N-(5-((4-(7-Cyano-1,3-dimethyl-1H-indol-5-yl)pyrimidin-2-yl)amino)-2-((2-(ethyl(methyl)amino)ethyl)(methyl)amino)-4-methoxyphenyl)acrylamide

To a solution of 5-(2-((5-amino-4-((2-(ethyl(methyl)amino)ethyl)(methyl)amino)-2-methoxyphenyl)amino)pyrimidin-4-yl)-1,3-dimethyl-1H-indole-7-carbonitrile (240 mg, crude) in DCM (4 mL) was added acryloyl chloride (102 g, 1.2 mmol, 1.5 eq) drop wise at −5 to 0° C. After addition, the reaction was warmed to RT and stirred for 1 hour. The reaction was diluted with DCM (10 mL), washed with saturated NaHCO₃ (5 mL), water (5 mL) and brine (5 mL). The combined organic layers were dried and concentrated to give the crude product, which was purified by Prep-HPLC to give N-(5-((4-(7-cyano-1,3-dimethyl-1H-indol-5-yl)pyrimidin-2-yl)amino)-2-((2-(ethyl(methyl)amino)ethyl)(methyl)amino)-4-methoxyphenyl)acrylamide (30 mg, 11%). ¹H NMR (300 MHz, DMSO-d₆): δ 9.91 (br, 1H), 9.08 (s, 1H), 8.71 (s, 1H), 8.51-8.48 (m, 2H), 8.15 (s, 1H), 7.57 (d, J=5.2 Hz, 1H), 7.32 (s, 1H), 7.03 (s, 1H), 6.43-6.39 (m, 1H), 6.27-6.23 (m, 1H), 5.75-5.73 (m, 1H), 4.05 (s, 3H), 3.87 (s, 3H), 2.88-2.87 (m, 2H), 2.71 (s, 3H), 2.48-2.45 (m, 5H), 2.34 (s, 3H), 2.21-2.14 (m, 2H), 1.01 (t, J=7.2 Hz, 3H). ESI-MS (m/z): 553.2 (M+H)⁺.

### Example 27. N-(2-((2-(Bis(methyl-d₃)amino)ethyl)(methyl)amino)-5-((4-(7-cyano-1,3-dimethyl-1H-indol-5-yl)pyrimidin-2-yl)amino)-4-methoxyphenyl)acrylamide mesylate

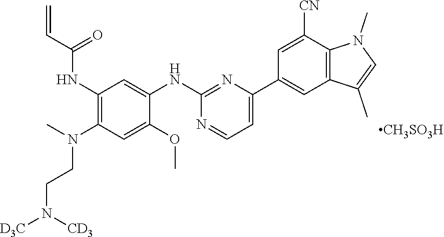

### tert-Butyl (2-(bis(methyl-d3)amino)ethyl)(methyl)carbamate

Under a nitrogen atmosphere, deuterated dimethylamine hydrochloride (9.0 g, 102.7 mmol) was added to 200 mL of 1,2-dichloroethane in a 500 mL three-necked flask at room temperature, then followed by addition of tert-butyl N-methyl-N-(2-oxoethyl)carbamate (17.8 g, 102.9 mmol) to reaction system. The reaction was stirred at room temperature for 2 h. After the reaction system was cooled to 0° C., sodium triacetoxyborohydride (32.6 g, 153.8 mmol) was added in batches and the reaction was then allowed to warm up to room temperature. The reaction mixture was stirred for overnight. The reaction was quenched with 100 mL of saturated aqueous solution of ammonium chloride, and the mixture was extracted with 200 mL of methylene chloride twice. The aqueous phases were collected, adjusted to pH 9 with saturated aqueous solution of sodium carbonate, extracted with 150 mL of methylene chloride twice, and the organic phases were combined, washed with 100 mL of saturated brine twice, dried over anhydrous sodium sulfate and concentrated to dryness to give 3.7 g of tert-butyl (2-(bis (methyl-d₃)amino)ethyl)(methyl)carbamate (17.3%) as yellow oil.

N²-Methyl-N¹,N¹-bis(methyl-d₃)ethane-1,2-diamine trifluoroacetate

Tert-butyl (2-(bis(methyl-d₃)amino)ethyl)(methyl)carbamate (3.7 g, 17.8 mmol) as a raw material was dissolved in 20 mL of anhydrous DCM in a 50 mL single-necked flask at room temperature, followed by adding 10 mL of TFA into the reaction system at room temperature. The reaction mixture was stirred for 4 h at room temperature. The reaction mixture was concentrated to give the intermediate as crude product that was used directly for the next step without purification.

### 5-(2-((4-Fluoro-2-methoxy-5-nitrophenyl)amino)pyrimidin-4-yl)-1,3-dimethyl-1H-indole-7-carbonitrile

To a solution of 5-(2-chloropyrimidin-4-yl)-1,3-dimethyl-1H-indole-7-carbonitrile (5.0 g, 17.7 mmol, 1.0 eq) and 4-fluoro-2-methoxy-5-nitroaniline (3.6 g, 19.4 mmol, 1.1 eq) in 2-pentanol (100 mL), was added p-toluenesulfonic acid monohydrate (3.3 g, 19.2 mmol, 1.1 eq) in a 250 mL single-necked flask. The mixture was heated to 118° C. for 8 h, then cooled to 25° C., and filtered under reduced pressure. The filter cake was washed with ethyl acetate (200 mL×2). The resulting filter cake was added with sodium bicarbonate solution to adjust the pH to 8 and the solid precipitated was filtered under vacuum. The filter cake was dried to obtain the product (6.9 g, yield 90.2%).

### 5-(2-((4-((2-(Bis(methyl-d3)amino)ethyl)(methyl)amino)-2-methoxy-5-nitrophenyl)amino)pyrimidin-4-yl)-1,3-dimethyl-1H-indole-7-carbonitrile

Crude product of N²-methyl N¹,N¹-bis(methyl-d₃)ethane-1,2-diamine trifluoroacetate and potassium carbonate (8.8 g, 63.8 mmol) were added to a solution of 5-(2-((4-fluoro-2-) methoxy-5-nitrophenyl)amino)pyrimidin-4-yl)-1,3-dimethyl-1H-indole-7-carbonitrile (6.9 g, 16.0 mol) in 100 mL of NMP in a 250 mL single-necked flask. The resulting mixture was stirred at 85° C. for 12 hours then cooled to r.t. Water (100 mL) was then added. Solid material was collected by filtration, washed with water (10 mL×2), and the filter cake was purified by silica gel column chromatography (DCM/MeOH/NH₃.H₂O=10/1/0.01) to give the product (5.7 g, yield 68.4%).

### 5-(2-((5-Amino-4-((2-(bis(methyl-d3)amino)ethyl) (methyl)amino)-2-methoxyphenyl)amino)pyrimidin-4-yl)-1,3-dimethyl-1H-indole-7-carbonitrile

To a solution of 5-(2-((4-((2-(bis(methyl-d₃)amino)ethyl)(methyl)amino)-2-methoxy-5-nitrophenyl)amino)pyrimidin-4-yl)-1,3-dimethyl-1H-indole-7-carbonitrile (5.6 g, 10.8 mmol) in THF (100 mL), was added Pd/C (800 mg). The mixture was stirred under 1 atm hydrogen atmosphere at 30° C. for 8 h in a 250 mL single-necked flask. The reaction mixture was filtered through celite and the filter cake was washed with DCM (20 mL×2), and the filtrate was concentrated to dryness under reduced pressure to give the product (4.5 g, 84.9% yield).

### N-(2-((2-(bis(methyl-d3)amino)ethyl)(methyl)amino)-5-((4-(7-cyano-1,3-dimethyl-1H-indol-5-yl)pyrimidin-2-yl)amino)-4-methoxyphenyl)acrylamide

Triethylamine (1.23 g, 12.2 mmol) was added to a solution of 5-(2-((5-amino-4-((2-(bis(methyl-d3)amino)ethyl) (methyl)amino)-2-methoxyphenyl)amino)pyrimidin-4-yl)-1,3-dimethyl-1H-indole-7-carbonitrile (3.0 g, 6.1 mmol) in 100 mL of THF in a 250 mL single-necked flask. Then acryloyl chloride (775 mg, 8.6 mmol) was added dropwise at −5° C. and stirring for 3 hours. The reaction was quenched with 5 mL saturated sodium carbonate solution. Then water (100 mL) was added, and the mixture was extracted with dichloromethane (200 mL). The organic phase was concentrated and the residue was purified by medium pressure preparative chromatography (H₂O/CH₃CN=1/9) to give the product (1.5 g, yield 45.1%).

### N-(2-((2-(bis(methyl-d3)amino)ethyl)(methyl)amino)-5-((4-(7-cyano-1,3-dimethyl-1H-indol-5-yl)pyrimidin-2-yl)amino)-4-methoxyphenyl)acrylamide mesylate

A solution of methanesulfonic acid (269 mg, 2.8 mmol) in purified water (2.7 mL) was added dropwise to a solution of N-(2-((2-(bis(methyl-d₃)amino)ethyl)(methyl)amino)-5-((4-(7-cyano-1,3-dimethyl-1H-indol-5-yl)pyrimidin-2-yl)amino)-4-methoxyphenyl)acrylamide (1.5 g, 2.8 mmol) in 15 mL of acetonitrile at 25° C. The mixture was stirred for 1 hour, and then heated up to 55° C. with stirring for 2 hours. The solvent was removed and the residue was grinded to obtain the product (1.73 g, yield 96.4%). ¹H-NMR (400 MHz, DMSO) δ: 9.53 (s, 1H), 9.16 (s, 1H), 8.77-8.68 (m, 2H), 8.53-8.48 (m, 2H), 8.30 (s, 1H), 7.61 (d, J=5.2 Hz, 1H), 7.34 (s, 1H), 7.02 (s, 1H), 6.72-6.60 (m, 1H), 6.35-6.27 (m, 1H), 5.81-5.76 (m, 1H), 4.04 (s, 3H), 3.90 (s, 3H), 3.30-3.20 (m, 4H), 2.61 (s, 3H), 2.33-2.2 (m, 6H). LC-MS (M/e): 545.4 (M-MSA+H⁺).

### Example 28. N-(5-((4-(7-Cyano-1,3-dimethyl-1H-indol-5-yl)pyrimidin-2-yl)amino)-2-((2-(dimethylamino)ethyl)(methyl)amino)-4-methoxyphenyl)acrylamide

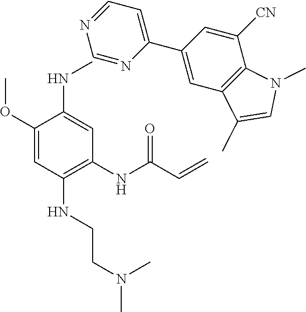

### 5-(2-((4-((2-(Dimethylamino)ethyl)amino)-2-methoxy-5-nitrophenyl)amino)pyrimidin-4-yl)-1,3-dimethyl-1H-indole-7-carbonitrile

To a solution of N, N-dimethylethane-1,2-diamine (3.2 g, 36.0 mmol) in DMF (100 mL), were added 5-(2-((4-fluoro-2-methoxy-5-nitrophenyl)amino)pyrimidin-4-yl)-1,3-dimethyl-1H-indole-7-carbonitrile (10.0 g, 24.0 mmol) and potassium carbonate (6.6 g, 48.0 mmol). The mixture was heated at 100° C. for 9 h before being poured into water (500 mL). After cooling, the solid product was collected by filtration, and purified by column chromatography to give the product (5.0 g, 43.2%). ESI-MS (m/z): 501.1 (M+H)⁺.

### tert-Butyl (4-((tert-butoxycarbonyl)(2-(dimethylamino)ethyl)amino)-2-methoxy-5-nitrophenyl)(4-(7-cyano-1,3-dimethyl-1H-indol-5-yl)pyrimidin-2-yl)carbamate

To a 100-mL flask, were added 5-(2-((4-((2-(dimethylamino)ethyl)amino)-2-methoxy-5-nitrophenyl)amino)pyrimidin-4-yl)-1,3-dimethyl-1H-indole-7-carbonitrile (5.0 g, 0.01 mol), Boc₂O (21.8 g, 0.1 mol), N,N-dimethylaminopyridine (12.2 g, 0.1 mol) and 1,4-dioxane (50 mL). The mixture was heated at reflux for 7 h before being cooled to 25° C. After concentration, the residue was purified by column chromatography (eluted with CH₂Cl₂/MeOH=25:1) to give 2.7 g (38.6%) of the desired product. ESI-MS (m/z): 701.4 (M+H)⁺.

### tert-Butyl (5-amino-4-((tert-butoxycarbonyl)(2-(dimethylamino)ethyl)amino)-2-methoxyphenyl)(4-(7-cyano-1,3-dimethyl-1H-indol-5-yl)pyrimidin-2-yl)carbamate

To a solution of tert-butyl (4-((tert-butoxycarbonyl)(2-(dimethylamino)ethyl)amino)-2-methoxy-5-nitrophenyl)(4-(7-cyano-1,3-dimethyl-1H-indol-5-yl)pyrimidin-2-yl)carbamate (2.0 g, 2.85 mmol) in 20 mL of tetrahydrofuran, was added 10% Pd/C (0.2 g, 50% wet). The mixture was hydrogenated for 7 h at 30° C. and atmospheric pressure. After filtration and concentration, the desired product (1.7 g, 87.7%) was obtained. ESI-MS (m/z): 671.4 (M+H)⁺.

### tert-Butyl (5-acrylamido-4-((tert-butoxycarbonyl)(2-(dimethylamino)ethyl)amino)-2-methoxyphenyl)(4-(7-cyano-1,3-dimethyl-1H-indol-5-yl)pyrimidin-2-yl)carbamate

To a solution of tert-butyl (5-amino-4-((tert-butoxycarbonyl)(2-(dimethylamino)ethyl)amino)-2-methoxyphenyl)(4-(7-cyano-1,3-dimethyl-1H-indol-5-yl)pyrimidin-2-yl)carbamate (1.7 g, 2.5 mmol) in CH₂Cl₂ (17 mL) and tetrahydrofuran (17 mL) at 0° C., were added triethylamine (0.51 g, 5.0 mmol) and acryloyl chloride (0.34 g, 3.75 mmol). The mixture was stirred for 2 h before being poured into 100 mL of saturated NaHCO₃ solution. Organic phase was separated, and aqueous phase was extracted with CH₂Cl₂ (100 mL). The combined organic phase was concentrated. The residue was purified by chromatography to give the desired product (1.01 g, 55%). ESI-MS (m/z): 725.5 (M+H)⁺.

### N-(5-((4-(7-cyano-1,3-dimethyl-1H-indol-5-yl)pyrimidin-2-yl)amino)-2-((2-(dimethylamino)ethyl)amino)-4-methoxyphenyl)acrylamide

Trifluoroacetic acid (10 mL) was added to tert-butyl (5-acrylamido-4-((tert-butoxycarbonyl)(2-(dimethylamino)ethyl)amino)-2-methoxyphenyl)(4-(7-cyano-1,3-dimethyl-1H-indol-5-yl)pyrimidin-2-yl)carbamate (1.01 g, 1.39 mmol) in a 25-mL flask. After stirring for 1 h, the mixture was concentrated and dissolved in CH₂Cl₂ (50 mL). The solution was washed with saturated sodium bicarbonate solution, dried over Na₂SO₄. Concentration afforded the product (400 mg, 54.7%). ¹HNMR (400 MHz, DMSO-d₆): δ 9.42 (s, 1H), 8.62 (s, 1H), 8.38 (s, 1H), 8.37 (d, J=5.2 Hz, 1H), 8.04 (s, 1H), 7.75 (s, 1H), 7.44 (d, J=5.2 Hz, 1H), 7.30 (s, 1H), 6.48 (m, 1H), 6.41 (s, 1H), 6.19 (dd, J₁=16.8 Hz, J₂=2.0 Hz, 1H), 5.69 (dd, J=10 Hz, J₂=2.0 Hz, 1H), 4.75 (m, 1H), 4.02 (s, 3H), 3.82 (s, 3H), 3.18 (m, 2H), 2.48 (bt, 2H), 2.28 (s, 3H), 2.17 (s, 3H). ESI-MS (m/z): 525.3 (M+H)⁺.

### Example 29. N-(5-((4-(7-Cyano-1,3-dimethyl-1H-indol-5-yl)pyrimidin-2-yl)amino)-4-methoxy-2-(methyl(2-(methylamino)ethyl)amino)phenyl)acrylamide

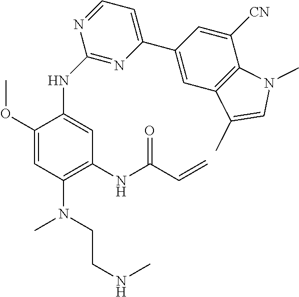 [SMILES: C=CC(=O)Nc1cc(Nc2nccc(-c3cc(C#N)c4c(c3)c(C)cn4C)n2)c(OC)cc1N(C)CCNC]

### tert-Butyl (2-((4-((4-(7-cyano-1,3-dimethyl-1H-indol-5-yl)pyrimidin-2-yl)amino)-5-methoxy-2-nitrophenyl)(methyl)amino)ethyl)(methyl)carbamate

To a solution of N-tert-butoxycarbonyl-N,N′-dimethylethane-1,2-diamine (6.5 g, 34.7 mmol) in DMF (90 mL), were added 5-(2-((4-fluoro-2-methoxy-5-nitrophenyl)amino)pyrimidin-4-yl)-1,3-dimethyl-1H-indole-7-carbonitrile (10.0 g, 24.0 mmol) and potassium carbonate (9.6 g, 69.3 mmol). The mixture was heated at 80° C. for 6 h before being poured into water (600 mL). The solid product was collected by filtration, and washed with water. After drying at 40° C. overnight, the product (8.6 g, 61.9%) was obtained. ESI-MS (m/z): 601.4 (M+H)⁺.

### tert-Butyl (2-((2-amino-4-((4-(7-cyano-1,3-dimethyl-1H-indol-5-yl)pyrimidin-2-yl)amino)-5-methoxyphenyl)(methyl)amino)ethyl)(methyl)carbamate

To a solution of tert-butyl (2-((4-((4-(7-cyano-1,3-dimethyl-1H-indol-5-yl)pyrimidin-2-yl)amino)-5-methoxy-2-nitrophenyl)(methyl)amino)ethyl)(methyl)carbamate (8.6 g, 14.3 mmol) in 170 mL of tetrahydrofuran, was added 10% Pd/C (1.72 g, 50% wet). The mixture was hydrogenated for 15 h at 30° C. and atmospheric pressure. After filtration and concentration, the desired product (8.2 g, 100%) was obtained. ESI-MS (m/z): 571.4 (M+H)⁺.

### tert-Butyl (2-((2-acrylamido-4-((4-(7-cyano-1,3-dimethyl-1H-indol-5-yl)pyrimidin-2-yl)amino)-5-methoxyphenyl)(methyl)amino)ethyl)(methyl)carbamate

To a solution of tert-butyl (2-((2-amino-4-((4-(7-cyano-1,3-dimethyl-1H-indol-5-yl)pyrimidin-2-yl)amino)-5-methoxyphenyl)(methyl)amino)ethyl)(methyl)carbamate (7.7 g, 13.5 mmol) and triethylamine (2.7 g, 27.0 mmol) in CH₂Cl₂ (60 mL) and tetrahydrofuran (20 mL) at 0° C., was added acryloyl chloride (1.7 g, 18.9 mmol) in 40 mL of tetrahydrofuran. The mixture was stirred for 4 h before being poured into a saturated NaHCO₃ solution. Organic phase was separated, and the aqueous phase was extracted with ethyl acetate. The combined organic phase was concentrated. The residue was purified by chromatography to give the desired product (4.8 g, 56.9%). ESI-MS (m/z): 625.4 (M+H)⁺.

### N-(5-((4-(7-cyano-1,3-dimethyl-1H-indol-5-yl)pyrimidin-2-yl)amino)-4-methoxy-2-(methyl(2-(methylamino)ethyl)amino)phenyl)acrylamide

Trifluoroacetic acid (40 mL) was added to a solution of tert-butyl (2-((2-acrylamido-4-((4-(7-cyano-1,3-dimethyl-1H-indol-5-yl)pyrimidin-2-yl)amino)-5-methoxyphenyl)(methyl)amino)ethyl)(methyl)carbamate (4.6 g, 7.4 mmol) in 80 mL of CH₂Cl₂. After stirring for 2 h at 30° C., the mixture was concentrated and was dissolved in CH₂Cl₂ (200 mL). The solution was washed with saturated sodium bicarbonate solution until pH=9, dried over Na₂SO₄. Concentrated and slurried with ethyl acetate to afford the product (1.6 g, 41.5%). ¹H NMR (400 MHz, DMSO-d₆): δ 9.36 (s, 1H), 8.84 (s, 1H), 8.66 (s, 1H), 8.48 (d, J=5.2 Hz, 1H), 8.46 (s, 1H), 8.17 (s, 1H), 7.57 (d, J=5.2 Hz, 1H), 7.29 (s, 1H), 6.97 (s, 1H), 6.72 (m, 1H), 6.29 (dd, J₁=16.8 Hz, J₂=1.6 Hz, 1H), 5.76 (dd, J₁=6.0 Hz, J₂=2.0 Hz, 1H), 4.02 (s, 1H), 3.84 (s, 1H), 3.20 (m, 2H), 3.11 (m, 2H), 2.67 (s, 3H), 2.60 (s, 3H), 2.28 (s, 1H). ESI-MS (m/z): 525.3 (M+H)⁺.

### Example 30. N-(5-(4-(7-Cyano-3-methyl-1H-indol-5-yl)pyrimidin-2-ylamino)-2-((2-(dimethylamino)ethyl)(methyl)amino)-4-methoxyphenyl) acrylamide

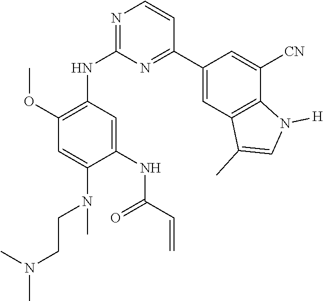 [SMILES: C=CC(=O)Nc1cc(Nc2nccc(-c3cc(C#N)c4[nH]cc(C)c4c3)n2)c(OC)cc1N(C)CCN(C)C]

To a solution of compound 7-cyano-5-(2-chloropyrimidin-4-yl)-3-methyl-1H-indole (130 mg, 0.48 mmol, 1.0 eq) and 5-(N-acrylamido)-4-(N, 1-(2-(N,N-dimethylamino)ethyl)-N, 1-methyl)amino)-2-methoxyaniline (141 mg, 0.48 mmol, 1.0 eq) in 2-pentanol (6.6 mL) was added p-toluenesulfonic acid monohydrate (101.6 mg, 0.528 mmol, 1.1 eq). The mixture was heated to 80° C. for 5 h. After cooling down to rt, the mixture was poured into water (50 mL), extracted with DCM (50 mL×3), the combined organic layers were washed with brine (50 mL), dried over sodium sulfate, concentrated and purified by silica column affording desired product N-(5-(4-(7-cyano-3-methyl-1H-indol-5-yl)pyrimidin-2-ylamino)-2-((2-(dimethylamino)ethyl)(methyl)amino)-4-methoxyphenyl) acrylamide (148 mg, 58%). ¹HNMR (300 MHz, DMSO-d₆): δ 11.90 (br, 1H), 10.19 (br, 1H), 9.14 (s, 1H), 8.73 (s, 1H), 8.52-8.49 (m, 2H), 8.15 (s, 1H), 7.67 (s, 1H), 7.33 (s, 1H), 7.04 (s, 1H), 6.36-6.29 (m, 2H), 5.75-5.72 (m, 1H), 3.86 (s, 3H), 2.86-2.84 (m, 2H), 2.71 (s, 3H), 2.31-2.29 (m, 5H), 2.21 (s, 6H). LCMS (M+H)⁺: 524.8. HPLC: 95.1%.

### BIOLOGY

### Abbreviations

DMSO: dimethylsulfoxide
DTT: dithiothreitol
ATP: adenosine triphosphate34
EDTA: ethylenediaminetetraacetic acid
Ki: enzyme inhibition constant
DMEM: Dulbecco's Modified Eagle Medium
NCS: newborn calf serum
PBS: phosphate buffered saline
PMSF; phenylmethanesulfonyl fluoride
ELISA: enzyme-linked immunosorbent assay
IgG; immunoglobulin G
FBS: fetal bovine serum
BDNF; brain derived neurotrophic factor

### Kinase Inhibition Assays

Kinase inhibition by the compounds of the invention is measured using commercially available assay kits and services that are well-known to a person having ordinary skill in the art. These kits and services are used to measure the inhibition of a variety of kinases, including without limitation ALK, ABL, AXL, Aur B & C, BLK, erbB-2, erbB-4. EGFR, mutant EGFR, HPK, IRAK1, RON, ROS1, SLK, STK10, TIE2, TRK, c-Met, Lck, Lyn, Src, Fyn, Syk, Zap-70, Itk, Tec, Btk, EGFR, ErbB2, Kdr, Flt-1, Flt-3, Tek, c-Met, InsR, and Atk. Commercial suppliers of these assay kits and services include Promega Corporation and Reaction Biology Corporation, EMD Millipore, and CEREP. In addition to the commercially available assay kits and services, the kinase inhibition activity of the compounds of formulae (I-VIII) is measured by way of the assays described below.

Purification of Epidermal Growth Factor Receptor Tyrosine Kinase Human EGF receptor tyrosine kinase is isolated from A431 human epidermoid carcinoma cells which overexpress EGF receptor by the following methods. Cells are grown in roller bottles in 50% Delbuco's Modified Eagle and 50% HAM F-12 nutrient media (Gibco) containing 10% fetal calf serum. Approximately 10⁹ cells are lysed in two volumes of buffer containing 20 mM 2-(4N-[2-hydroxyethyl]piperazin-1-yl)ethanesulfonic acid (hepes), pH 7.4, 5 mM ethylene glycol bis(2-aminoethyl ether) N,N,N′,N′-tetraacetic acid, 1% Triton X-100, 10% glycerol, 0.1 mM sodium orthovanadate, 5 mM sodium fluoride, 4 mM pyrophosphate, 4 mM benzamide, 1 mM dithiothreitol, 80 μg/mL aprotinin, 40 μg/mL leupeptin and 1 mM phenylmethylsulfonyl fluoride. After centrifugation at 25,000×g for 10 minutes, the supernatant is equilibrated for 2 h at 40° C. with 10 mL of wheat germ agglutinin sepharose that was previously equilibrated with 50 mM Hepes, 10% glycerol, 0.1% Triton X-100 and 150 mM NaCl, pH 7.5, (equilibration buffer). Contaminating proteins are washed from the resin with 1M NaCl in equilibration buffer, and the enzyme was eluted with 0.5 M N-acetyl-1-D-glucosamine in equilibration buffer, followed by 1 mM urea. The enzyme are eluted with 0.1 mg/ml EGF. The receptor appears to be homogeneous as assessed by Coomassie blue stained polyacrylamide electrophoretic gels.

Using the same technique as described in the previous paragraph, various mutated forms of the epidermal growth factor receptor may be isolated from appropriate cell lines which contain them. For example, the EGFR del746-750 mutant protein may be extracted from PC-9 cells, and the L858R/T790M double mutant EGFR protein may be isolated from H1975 cells.

Determination of IC50 Values for Single Mutant EGFRz_d746-750

Enzyme assays for IC50 determinations are performed in a total volume of 25 μL. Dilute all compounds to 500 μM stock solutions in 100% DMSO and make a serial of 4-fold dilution for 10 doses. “Max” and “Min” control contain 100% DMSO. “Max” stands for DMSO control without enzyme, “Min” stands for low control without compounds. Transfer 10 μl of compounds to 90 μl of 1× kinase base buffer to make intermediate dilution. Transfer 5 μl of intermediate dilution compounds to the 384-well assay plate, then 10 μl 2.5× enzyme buffer containing (12.5 nM EGFR_d746-750, 5 mM DTT, 1× kinase base buffer) are added to assay plate. Incubate at RT for 10 minutes and add 10 μl 2.5× substrate buffer containing (7.5 μM Peptide, 35 μM ATP, 25 mM MgCl2, 1× kinase base buffer) to start reaction. Incubate at RT for 1 hr and 25 μl stop buffer to end up reaction. Collect conversion data from Caliper and conversion data from Caliper program. Fit the data in XLfit to obtain IC50 values.

Determination of IC50 Values for Double-Mutant EGFR (EGFR T790M/L858R)

Enzyme assays for IC50 determinations are performed in a total volume of 25 μL. Dilute all compounds to 500 μM stock solutions in 100% DMSO and make a serial of 4-fold dilution for 10 doses. “Max” and “Min” control contain 100% DMSO. “Max” stands for DMSO control without enzyme, “Min” stands for low control without compounds. Transfer 10 μl of compounds to 90 μl of 1× kinase base buffer to make intermediate dilution. Transfer 5 μl of intermediate dilution compounds to the 384-well assay plate, then 10 μl 2.5× enzyme buffer containing (25 nM EGFR_T790M/L858R, 5 mM DTT, 1× kinase base buffer) are added to assay plate. Incubate at RT for 10 minutes and add 10 μl 2.5× substrate buffer containing (7.5 μM Peptide, 47.5 μM ATP, 25 mM MgCl2, 1× kinase base buffer) to start reaction. Incubate at RT for 1 hr and 25 μl stop buffer to end up reaction. Collect conversion data from Caliper and conversion data from Caliper program. Fit the data in XLfit to obtain IC₅₀ values.

Determination of IC50 Values for wt EGFR

Enzyme assays for IC50 determinations are performed in a total volume of 25 μL. Dilute all compounds to 500 μM stock solutions in 100% DMSO and make a serial of 4-fold dilution for 10 doses. “Max” and “Min” control contain 100% DMSO. “Max” stands for DMSO control without enzyme, “Min” stands for low control without compounds. Transfer 10 μl of compounds to 90 μl of 1× kinase base buffer to make intermediate dilution. Transfer 5 μl of intermediate dilution compounds to the 384-well assay plate, then 10 μl 2.5× enzyme buffer containing (20 nM EGFR, 5 mM DTT, 1× kinase base buffer) are added to assay plate. Incubate at RT for 10 minutes and add 10 μl 2.5× substrate buffer containing (7.5 μM Peptide, 5.75 μM ATP, 25 mM MgCl2, 25 mM MnCl2, 1× kinase base buffer) to start reaction. Incubate at RT for 1 hr and 25 μl stop buffer to end up reaction. Collect conversion data from Caliper and conversion data from Caliper program. Fit the data in XLfit to obtain IC50 values.

Other Kinase Inhibition Assays

Assays to determine the inhibition of other kinases by the compounds of formulae (I-VIII) are performed according to procedures known to a person having ordinary skill in the art. These assays include, but are not limited to, assays directed to the inhibition of the following kinases:

Wild-type c-Met Kinase. Inhibition of wild-type c-Met kinase is determined as described in International Publication No. WO 2011/069761, the entire contents of which are incorporated by reference.

LCK and BLK Kinases. Inhibition of LCK and BLK kinases is determined as described in U.S. Pat. No. 7,125,875, the entire contents of which are incorporated by reference.

The compounds described herein are screened in the following manner. Kinases suitable for use in the following protocol to determine kinase activity of the compounds described herein include, but are not limited to: Lck, Lyn, Src, Fyn, Syk, Zap-70, Itk, Tec, Btk, ErbB2, ErbB-4, Kdr, Flt-1, Flt-3, Tek, c-Met, and Atk. Kinases are expressed as either kinase domains or full length constructs fused to glutathione S-transferase (GST) or polyHistidine tagged fusion proteins in either E. coli or Baculovirus-High Five expression systems. They are purified to near homogeneity by affinity chromatography essentially as previously described (Lehr et al., 1996; Gish et al., 1995). In some instances, kinases are co-expressed or mixed with purified or partially purified regulatory polypeptides prior to measurement of activity. Kinase activity and inhibition are measured essentially by established protocols (Braunwalder et al., 1996). Briefly,

The transfer of ³²P0₄ from ATP to the synthetic substrates poly(Glu-Tyr) 4:1 or poly(Arg-Ser) 3:1 attached to the bioactive surface of microtiter plates serves as the basis to evaluate enzyme activity. After an incubation period, the amount of phosphate transferred is measured by first washing the plate with 0.5% phosphoric acid, adding liquid scintillant, and then counting in a liquid scintillation detector. The IC₅₀ is determined by the concentration of compound that causes a 50% reduction in the amount of ³²P incorporated onto the substrate bound to the plate. Other similar methods whereby phosphate is transferred to peptide or polypeptide substrate containing tyrosine, serine, threonine, or histidine, either alone, in combination, or in combination with other amino acids, in solution or immobilized (i.e., solid phase) are also useful. For example, transfer of phosphate to a peptide or polypeptide can also be detected using scintillation proximity (Wu et al., 2000), ELISA (Cleaveland et al., 1990), Fluorescence Polarization (Seethala and Menzel, 1998), and homogeneous time resolved fluorescence (HTRF, Kolb et al., 1998). Alternatively, kinase activity can be measured using antibody-based methods whereby an antibody or polypeptide is used as a reagent to detect phosphorylated target polypeptide.

### REFERENCES

Braunwalder et al. (1996). Anal. Biochem. 234(1):23-26.
Cleaveland et al. (1990). Anal Biochem. 190(2):249-53.
Gish et al. (1995). Protein Eng. 8(6):609-614.
Kolb et al. (1998). Drug Discov. Today. 3:333-342.
Lehr et al. (1996). Gene 169(2):27527-9.
Seethala et al. (1998). Anal Biochem. 255(2):257-62.
Wu et al. (2000). Comb Chem High Throughput Screen. 3(1):27-36.

### EGFR Cell Assay Summary Protocols

Cell Proliferation Assays

H1975 Inhibition Assay (Cell Proliferation).

H1975 cells were cryopreserved in liquid nitrogen. Before thawing the cells, place 15 mL of cell culture medium (RPMI 1640 Medium supplied with 10% fetal bovine serum and 1% penicillin/streptomycin) into a T75 flask and pre-incubate the flask in humidified 37° C./5% CO₂ incubator for 15 minutes to allow medium to equilibrate to the proper pH and temperature. Remove the vial from liquid nitrogen and thaw rapidly by placing at 37° C. in a water bath with gentle agitation for 1-2 minutes and then decontaminated by wiping with 70% ethanol before opening in a Class II biological safety cabinet. Transfer the vial contents drop-wise into 10 mL of cell culture medium in a sterile 15 mL conical tube. Then centrifuged the tube at 200× g for 5 minutes and aspirate the supernatant. Re-suspend the cell pellet with 1 mL of fresh cell culture medium and transfer it in to the T75 flask containing cell culture medium.

To passage H1975 cells, firstly, the adherent cells were rinsed with Trypsin/EDTA. Then add Trypsin/EDTA (3 mL for a T75 flask) into the flask and swirl to ensure the cells coated with trypsin evenly. Then incubate the flasks at 37° C. until the cells detach. Add equal volume of cell culture medium to stop the reaction. Collect the detached cells and centrifuged at 200× g for 5 minutes followed by re-suspended in fresh culture medium. Then, the cells were transferred into a new T75 flask containing cell culture medium. Cells were sub-cultured three times per week at a ratio of 1:2 or 1:4 in culture medium.

Test compounds were dissolved in DMSO at 30 mM. 45 μL of compound was transferred into a 384-well compound source plate (LABCYTE cat # P-05525) and serially diluted at 1:3 ratio to create a 13-point dilutions. The same volume of DMSO was adopted as high control. 20 nL of these compounds DMSO dilutes (10 points, from 1.11 mM to 0.056 μM) were dispensed into a new 384-well assay plate by Echo 550.

Harvest cells from flask into cell culture medium as described above and the cell numbers were counted using Automated Cell Counter (Thermo Fisher Scientific, Countess™). Dilute the cells into 25,000 cells/mL with culture medium and add 40 μL of cell suspension into each well of 384-well cell culture plate as designated. The final concentration was 1,000 cells/well. Add medium only as low control. The plates were covered with lid and placed in 370C 5% CO₂ incubator for 72 hours.

After 72 hours incubation, remove the plates from incubator and equilibrate at room temperature for 15 minutes. Incubate the CellTiter Glo reagents (Promega, G9243) at 37° C. before the experiment. The buffer was equilibrated to room temperature and used to dissolve the substrate. To determine the cell viability, add 40 μL of CellTiter-Glo reagent into each well to be detected (at 1:1 to culture medium). Then the place the plates at room temperature for 30 min followed by read on EnSpire (PerkinElmer).

For estimation of IC₅₀, the luminescence readout are transformed to % Inhibition by applying the following equation:

$$\frac{{Lum}_{HC} - {Lum}_{Cpd}}{{Lumo}_{HC} - {Lum}_{LC}} .$$

The IC₅₀ was then calculated by fitting in XLFit to a four parameters logistic curve.

PC-9 Growth Inhibition Assay (Cell Proliferation).

The inhibition assay of PC-9 cells was conducted in the same manner as described above for H1975 cells.

A431 Inhibition Assay (Cell Proliferation).

The culture medium of A431 cells was Dulbecco's Modified Eagle Medium supplied with 10% fetal bovine serum and 1% penicillin/streptomycin. The DMSO dilutions used in A431 assay was 10 points from 30 nM to 1.52 uM. The rest procedure was conducted in the same manner as described above for H1975 cells.

TABLE 1
Cellular Proliferation Assay Results

| | | PC9 IC₅₀ | NCI-H1975 | A431 IC₅₀ |
|---|---|---|---|---|
| Run | Example# | (nM) SM | IC₅₀ (nM) DM | (nM) WT |
| 1 | AZD9291 | 5.21 | 10.50 | 458.90 |
| 1 | AZD5104 | 1.92 | 4.05 | 105.15 |
| 1 | Afatinib | 0.28 | 132.54 | 72.65 |
| 1 | 1 | 9.24 | 11.44 | 2137.56 |
| 1 | 2 | 4.18 | 4.77 | 2117.34 |
| 1 | 5 | 4.86 | 5.86 | 1299.19 |
| 2 | AZD9291 | 3.47 | 7.16 | 1107.44 |
| 2 | AZD5104 | 1.34 | 2.95 | 312.73 |
| 2 | Afatinib | 0.25 | 87.36 | 271.09 |
| 2 | 2 | 3.22 | 3.45 | 3680.72 |
| 2 | 4 | 1.91 | 1.03 | 1462.78 |
| 2 | 5 | 2.90 | 3.58 | 2212.15 |
| 2 | 6 | 1.82 | 2.60 | 1087.89 |
| 2 | 7 | 7.38 | 4.76 | 2172.28 |
| 2 | 18 | 1.79 | 2.66 | 1243.04 |
| 2 | 19 | 3.78 | 2.82 | 952.67 |
| 2 | 21 | 7.21 | 6.84 | 1713.10 |
| 2 | 22 | 1.79 | 3.57 | 675.49 |
| 3 | AZD9291 | 7.27 | 10.74 | 847.76 |
| 3 | AZD5104 | 2.27 | 3.06 | 132.72 |
| 3 | Afatinib | 0.32 | 138.59 | 107.76 |
| 3 | 3 | 4.15 | 4.38 | 1376.37 |
| 3 | 9 | 7.67 | 3.05 | 2203.53 |
| 3 | 11 | 7.73 | 6.51 | 2529.92 |
| 3 | 12 | 19.08 | 16.80 | 2430.04 |
| 3 | 20 | 0.91 | 1.08 | 97.62 |
| 4 | AZD-9291 | 7.43 | 10.47 | 718.96 |
| 4 | AZD-5104 | 1.57 | 3.90 | 95.20 |
| 4 | Afatinib | 0.53 | 430.67 | 70.84 |
| 4 | 23 | 3.87 | 7.35 | 566.17 |
| 4 | 24 | 1.57 | 2.43 | 217.19 |
| 4 | 26 | 6.51 | 5.64 | 2499.64 |
| 5 | 27 | — | 2.26 | — |
| 6 | 28 | 81.37 | 13.6 | 912.2 |
| 6 | 29 | 51.9 | 23.61 | 1370 |
| 7 | 30 | 2.07 | 1.05 | 505.52 |

TABLE 2
Cellular Proliferation Assay
Results for Comparative Examples

| | | PC9 IC₅₀ | NCI-H1975 IC₅₀ | A431 IC₅₀ |
|---|---|---|---|---|
| Run | Example# | (nM) SM | (nM) DM | (nM) WT |
| 3 | 8 | 18.59 | 11.56 | 1803.71 |
| 3 | 13 | 4.64 | 2.98 | 1362.43 |
| 4 | 10 | 16.52 | 12.68 | 694.74 |
| 4 | 25 | 16.19 | 12.58 | 1133.62 |

Cellular EGFR Autophosphorylation Assays.

L858R/T790M Double Mutant H1975 Autophosphorylation Inhibition Assay (ELISA)

H1975 cells were cryopreserved in liquid nitrogen. Before thawing the cells, 15 mL of cell culture medium (RPMI 1640 Medium supplied with 10% fetal bovine serum and 1% penicillin/streptomycin) were placed into a T75 flask and pre-incubate the flask in humidified 37° C./5% CO₂ incubator for 15 minutes to allow medium to equilibrate to the proper pH and temperature. The vial was removed from liquid nitrogen and thawed rapidly by placing at 37° C. in a water bath with gentle agitation for 1-2 minutes and then decontaminated by wiping with 70% ethanol before opening in a Class II biological safety cabinet. The vial's contents were transferred drop-wise into 10 mL of cell culture medium in a sterile 15 mL conical tube. Then centrifuged the tube at 200× g for 5 minutes and aspirate the supernatant. The cell pellet were re-suspended with 1 mL of fresh cell culture medium and transfer it in to the T75 flask containing cell culture medium.

To passage H1975 cells, firstly, the adherent cells were rinsed with Trypsin/EDTA. Then add Trypsin/EDTA (3 mL for a T75 flask) into the flask and swirl to ensure the cells coated with trypsin evenly. Then the flasks were incubated at 37° C. until the cells detach. Add equal volume of cell culture medium to stop the reaction. The detached cells were collected and centrifuged at 200×g for 5 minutes followed by re-suspended in fresh culture medium. Then, the cells were transferred into a new T75 flask containing cell culture medium. Cells were sub-cultured three times per week at a ratio of 1:4 in culture medium.

Cells from flask were harvested into cell culture medium and the cell numbers counted using Automated Cell Counter (Thermo Fisher Scientific, Countess™). The cells were dicluted into 250,000 cells/mL with culture medium and add 40 μL of cell suspension into each well of 384-well cell culture plate as designated. The final concentration was 10,000 cells/well. The plates were covered with lid and placed in 37° C. 5% CO₂ incubator overnight for cell attachment.

On the second day, test compounds were dissolved in DMSO at 10 mM. 45 uL of compound was transfer into a 384-well compound source plate (LABCYTE cat #P-05525) and serially diluted at 1:3 ratio to create a 13-point dilutions. The same volume of DMSO was adopted as high control. 40 nL of these compounds DMSO dilutes (11 points, from 1.11 mM to 0.019 uM) were dispensed into the H1975 cell plate by Echo 550.

The plate was placed back to 37° C. 5% CO₂ incubator for 2 hours. The medium of each well were replaced with ice-cold HBSS. Then the HBSS was removed, added 30 μL cell lysis buffer into each well and shake the plates for 30 mins on a plate shaker. Centrifuged for 5 min at 1,000 rpm to remove bubbles and transfer 25 uL of the lysate supernatant for p-EGFR assay by using a commercial ELISA kit (R&D, DYC1095B-5).

For estimation of IC₅₀, the absorption readout were transformed to % relative activity by applying the following equation:

$$\% Inhibition = \frac{{Abs}_{HC} - {Abs}_{cpd}}{{Abs}_{HC}} .$$

The IC₅₀ was then calculated by fitting in XLFit (IDBS, Guildford, Surrey) to a four parameters logistic curve.

Wild Type EGFR A431 Autophosphorylation Inhibition Assay (ELISA).

The culture medium of A431 cells was Dulbecco's Modified Eagle Medium supplied with 10% fetal bovine serum and 1% penicillin/streptomycin. The DMSO dilutions used in A431 assay was 11 points from 10 mM to 0.17 uM. After 2 hour treatment with test compounds, add 4.5 μL of EGF (1 μg/mL) into each well and stimulate for 10 min. The rest of the procedure was processed in the same manner as described above for H1975 cells.

Exon 19 Deletion EGFR (Activating Single Mutant) PC-9 Cellular Autophosphorylation Assay.

The human lung cell line PC9 (Exon 19 deletion EGFR) were obtained from the American type Culture Collection. PC9 cells were maintained in RPMI 1640, containing 10% fetal calf serum and 2 mM glutamine. Cells were grown in a humidified incubator at 37° C. with 5% CO₂, Assays to measure cellular phosphorylation of endogenous p-EGFR in cell Iysates were carried out according to the protocol described in the R&D Systems DuoSet IC Human Phospho-EGF R ELISA (R&D Systems catalogue number # DYCI095). 40 μL of cells were seeded (10000 cells/well) in growth medium in Coming black, clear-bottomed 384-well plates and incubated at 37° C. with 5% CO₂ overnight. Cells were acoustically dosed using an Echo 555, with compounds serially diluted in 100% DMSO. Plates were incubated for a further 2 h, then following aspiration of medium, 40 μL× lysis buffer is added to each well. Greiner black high bind 384 well plates were coated with capture antibody and then blocked with 3% BSA. Following removal of block, 15 μL of lysate are transferred to the Greiner black high bind 384 well plates and incubated for 2 hours. Following aspiration and washing of the plates with PBS, 20 μL of detection antibody were added and incubated for 2 hours. Following aspiration and washing of the plates with PBS, 20 μL of QuantaBlu fluorogenic peroxidase substrate (Thermo Fisher Scientific catalogue number 15169) were added and incubated for 1 hour. 20 μL QuantaBlu stop solution were added to plates and fluorescence read on an Envision plate reader using Excitation 352 nm wavelength and emission 460 nm wavelength. The data obtained with each compound are exported into a suitable software package (such as Origin) to perform curve fitting analysis. From this data an IC₅₀ value was determined by calculation of the concentration of compound that is required to give a 50% effect.

TABLE 3
Cellular Autophosphorylation Assay Results

| | | SM | DM | WT |
|---|---|---|---|---|
| RUN | EXAMPLE | (PC9) | (H1975) | (A431) |
| # | # | IC₅₀ nM | IC₅₀ nM | IC₅₀ nM |
| 1 | AZD-9291 | 27.02 | 45.90 | 563.75 |
| 1 | AZD-5104 | 5.83 | 4.36 | 24.21 |
| 1 | 2 | 5.40 | 2.54 | 165.69 |
| 2 | AZD-9291 | 15.97 | 12.87 | 137.25 |
| 2 | AZD-5104 | 4.04 | 2.47 | 8.87 |
| 2 | 4 | 10.91 | 1.98 | 69.46 |
| 2 | 5 | 17.11 | 6.46 | 148.18 |
| 2 | 6 | 8.35 | 7.52 | 72.48 |
| 2 | 7 | 33.05 | 8.26 | 302.05 |
| 2 | 18 | 5.87 | 3.94 | 49.08 |
| 2 | 19 | 14.75 | 5.35 | 92.78 |
| 2 | 21 | 23.83 | 12.17 | 192.08 |
| 2 | 22 | 5.79 | 4.54 | 35.00 |
| 3 | AZD-9291 | 13.39 | 20.85 | 119.87 |
| 3 | AZD-5104 | 4.64 | 5.69 | 14.56 |
| 3 | 3 | 5.15 | 2.16 | 63.12 |
| 3 | 20 | 1.38 | 1.07 | 19.02 |
| 4 | AZD-9291 | 12.42 | 16.29 | 114.84 |
| 4 | AZD-5104 | 5.59 | 7.46 | 15.18 |
| 4 | Afatinib | — | — | 3.31 |
| 4 | 2 | 7.06 | 4.29 | 77.13 |
| 4 | 9 | 10.85 | 5.71 | 139.68 |
| 4 | 12 | 11.79 | 7.73 | 63.55 |

TABLE 4
Cellular Autophosphorylation Assay
Results for Comparative Examples

| | | SM | DM | WT |
|---|---|---|---|---|
| RUN | EXAMPLE | (PC9) | (H1975) | (A431) |
| # | # | IC₅₀ nM | IC₅₀ nM | IC₅₀ nM |
| 3 | 8 | 27.57 | 20.69 | 326.76 |
| 4 | 8 | 29.39 | 38.84 | 464.30 |
| 4 | 13 | 7.57 | 4.39 | 64.19 |

IGF-1R Inhibition Assay

Test compound was dissolved in DMSO at 30 mM. 45 μL of compound was transfer into a 384-well compound source plate (LABCYTE cat # P-05525) and serially diluted at 1:3 ratio to create a 12-point dilutions. The same volume of DMSO was adopted as high control. 20 nL of these compounds DMSO dilutes were dispensed into a new 384-well assay plate by Echo 550. IGF-1R protein (0.87 nM, CARNA BIOSCIENCE, cat #08-141), florescent labeled substrate FLPeptide13 (2 μM, PerkinElmer, cat #760357) was prepared in kinase assay buffer (100 mM HEPES (pH 7.5), 10 mM MgCl₂, 0.05% Brij-35, 0.5 mM DTT and 0.1 mg/ml BSA). 15 μL of kinase assay buffer containing IGF-1R protein and substrate was transferred to assay plate and incubate at RT for 30 minutes. Kinase assay buffer supplemented with substrate peptides was employed as low control to monitor the background. 40 μM ATP was prepared in kinase assay buffer containing and 5 μL of ATP solution was added to each well to start the reaction. The assay plate was incubated at 25° C. for 180 minutes and the reaction was stopped by adding 40 μL of 0.5 M EDTA.

Phosphorylated fluorescent-tagged peptides were differentiated from non-phosphorylated peptides by separating using Caliper EZ Reader II and the detection was directly converted to conversion ratio.

For estimation of IC₅₀, the % substrate conversion values were transformed to % relative activity by applying the following equation:

$$\% relative activity = \frac{{Ratio}_{cpd} - {Ratio}_{LC}}{{Ratio}_{HC} - {Ratio}_{LC}} .$$

The IC₅₀ was then calculated by fitting in XLFit (IDBS, Guildford, Surrey) to a four parameters logistic curve.

INSR Inhibition Assay

Test compound was dissolved in DMSO at 30 mM. 45 uL of compound was transfer into a 384-well compound source plate (LABCYTE cat # P-05525) and serially diluted at 1:3 ratio to create a 12-point dilutions. The same volume of DMSO was adopted as high control. 20 nL of these compounds DMSO dilutes were dispensed into a new 384-well assay plate by Echo 550. INSR protein (0.73 nM, CARNA BIOSCIENCE, cat #08-142), florescent labeled substrate FLPeptide13 (2 μM, PerkinElmer, cat #760357) was prepared in kinase assay buffer (100 mM HEPES (pH 7.5), 10 mM MgCl₂, 0.05% Brij-35, 0.5 mM DTT and 0.1 mg/ml BSA). 15 μL of kinase assay buffer containing INSR protein and substrate was transferred to assay plate and incubate at RT for 30 minutes. Kinase assay buffer supplemented with substrate peptides was employed as low control to monitor the background. 40 μM ATP was prepared in kinase assay buffer containing and 5 μL of ATP solution was added to each well to start the reaction. The assay plate was incubated at 25° C. for 180 minutes and the reaction was stopped by adding 40 μL of 0.5 M EDTA.

The result was analyzed in the same manner as IGF-IR. See Table 5 for IGF-IR and INSR Enzyme Assay results.

TABLE 5
IGF-1R and INSR Enzyme Assay Results

| | | IC₅₀ (nM) | IC₅₀ (nM) |
|---|---|---|---|
| Run # | Example # | IGF-1R | INSR |
| 3 | AZD-9291 | 454.51 | 478.21 |
| 3 | AZD-5104 | 101.86 | 94.82 |
| 3 | Staurosporin | 22.52 | 13.09 |
| 3 | 2 | 1121.53 | 1823.41 |
| 3 | 4 | 830.60 | 965.32 |
| 3 | 5 | 1590.55 | 2139.47 |
| 3 | 6 | 2092.88 | 2306.29 |
| 3 | 7 | 390.46 | 621.34 |
| 3 | 18 | 1159.39 | 968.43 |
| 3 | 19 | 1683.43 | 1745.82 |
| 3 | 21 | 1511.37 | 2731.05 |
| 3 | 22 | 5646.59 | 2525.18 |

Various Other Kinases. Inhibition of various other kinases, including but not limited to Lck, Lyn, Src, Fyn, Syk, Zap-70, Itk, Tec, Btk, EGFR, ErbB2, Kdr, Flt-1, Flt-3, Tek, c-Met, InsR, and Atk is determined as described in U.S. Pat. No. 6,881,737, the entire contents of which are incorporated by reference.

Mouse In Vivo PK Study

To determine the drug concentration in plasma of the compounds of the present disclosure following intravenous and oral administration in male CD 1 Mice, pharmacokinetic profile and PK parameters were obtained.

Study Protocol:

Test animals: healthy male CD₁ mice (body weight 20-30 g, 18 mice, free access to food and water), provided by Sibeifu laboratory.

Dose Level and Dose Route: dosed the animals via intravenous injection from tail vein for IV group (1 mg/kg, 5 mL/kg, 10% DMSO/40% PEG400/50% water), dosed the animals via oral gavage for PO group (10 mg/kg, 10 mL/kg, (10% DMSO/40% PEG400/50% water), respectively.

Samples collection: the healthy animals were used, weighed the bodyweight and marked at tail and cage card prior to dosing. Blood samples (0.03 mL per time point) were collected from dorsal metatarsal vein at 0.083, 0.25, 0.5, 1, 2, 4, 8, 24 h post dose for IV group and at 0.25, 0.5, 1, 2, 4, 8, 24 h post dose for PO group, the terminal time point was collected from heart (˜0.3 mL). The blood samples were put into the tube with heparin-Na coated and then put on the cold box, centrifuged at 4° C. 4000 g, 5 minutes immediately after collecting all samples per time point to get plasma. The plasma samples were stored in a freezer at −75±15° C. prior to analysis.

The drug concentration was determined by LC/MS/MS method, and the PK parameters were observed as follows.

TABLE 6
PK Parameters in Mouse

| PK | | | | Example | Example | Example | Example |
|---|---|---|---|---|---|---|---|
| parameters | Unit | Example 6 | Example 2* | 9 | 11 | 4 | 27 |
| Cl | mL/min/kg | 100 | 35.5 | 67 | 114 | 183 | 60.7 |
| T_{1/2} (IV) | h | 0.92 | 1.50 | 0.53 | 1.1 | 0.576 | 1.76 |
| T_{1/2} (PO) | h | 2.67 | 2.57 | 3.55 | 5.06 | 7.3 | 3.65 |
| Cₘₐₓ (PO) | ng/mL | 117 | 119 | 159 | 97 | 80.2 | 99 |
| AUC (IV) | h*ng/mL | 168 | 937 | 249 | 153 | 96 | 297 |
| AUC (PO) | h*ng/mL | 784 | 976 | 719 | 641 | 225 | 1037 |
| F | % | 46.7 | 20.8 | 23.1 | 29.3 | 15 | 31.4 |

Dosing 1 mg/kg IV; 10 mg/kg PO; *For example 2, Dosing 2 mg/kg IV; 10 mg/kg PO

TABLE 7
PK Parameters in Mouse for Comparative Example

| PK parameters | Unit | Example 13 |
|---|---|---|
| Cl | mL/min/kg | 99 |
| T_{1/2} (IV) | h | 1.52 |
| T_{1/2} (PO) | h | 6.10 |
| Cₘₐₓ (PO) | ng/mL | 63.5 |
| AUC (IV) | h*ng/mL | 169 |
| AUC (PO) | h*ng/mL | 486 |
| F | % | 17.3 |

Dosing 1 mg/kg IV 10 mg/kg PO

Animal Xenograft Tumor Models

General Protocol.

Appropriately transformed cells, either from ATCC cell lines, known to carry the oncogene of interest, or from deliberate transfections, are suspended in appropriate media, and 5×10⁶ or 1×10⁷ cells are injected into the flank of nu/nu mice. Alternatively trochar placement of fragments of in vivo passaged tumors, usually about 1 mm³ can be used to initiate the tumors. When tumors have reached an appropriate size for the experiment, usually in the 100-300 mg range, animals are randomized into matched groups of 6-10 mice, and tumor size and given vehicle or test article by perioral gavage once or twice daily. Tumor volumes are determined using calipers. The percentage increase in the volume of a xenograft tumor on day n versus day 0 (the day when dosing of the test compound began) is calculated as (tumor volume on day n−tumor volume on day 0/tumor volume on day 0)×100. The mean percentage of tumor growth inhibition in each drug-treated group relative to the vehicle-treated group is calculated as (1−mean percent increase of tumor volume in the drug-treated group/mean percent increase of the tumor volume in the vehicle-treated group)×100. Statistical significance is evaluated using a one-tailed t test.

Wild-type EGFR xenograft Assay. For determination of efficacy against tumors overexpressing wt EGFR, xenografts grown from either A431 epidermoid or LoVo colon carcinoma cells may be used.

EGFR del746-750 xenograft model. For determination of efficacy against tumors overexpressing EGFR-del746-750, xenografts grown from PC9 NSCLC cells may be used.

EGFR L858R xenograft model. For determination of efficacy against tumors overexpressing EGFR-L858R, xenografts grown from H3255 NSCLC cells may be used.

EGFR L858R/T790M double-mutant xenograft model. For determination of efficacy against tumors overexpressing EGFR-L858R/T790M double mutant, xenografts grown from H1975 NSCLC cells were used. Tumor size was measured on day 10 after dosing. The effect of the compound of the present disclosure on the tumor size for the H1975 xenograft model is represented in Table 8.

Pharmacodynamic Assays.

Mice bearing any of the above tumors, preferably of 200-300 mg size, can be euthanized at appropriate intervals after oral administration of drug. The tumors are excised, snap-frozen, and dispersed using a Qiagen Tissue-Lyser in a nondenaturing lysis buffer containing protease and phosphatase inhibitors. The homogenate is lysed at 4° C. for 1 h, clarified by centrifugation, and then analyzed by quantitative Western blotting for phosphor EGFR/erbB-2/3/4 and total receptor. The phospho-RTK signal of each RTK band is normalized with its total RTK signal. Alternatively, the ratio of total ERK to phosphor-ERK can be measured in the tumors by similar techniques, using the appropriate eERK and phosphor-ERK antibodies.

TABLE 8
H1975 Xenograft Model (Tumor
size measured on day 10 after dosing)

| | Tumor Size^{a} | T_{RTV}/ | TGI^{c} |
|---|---|---|---|
| Group | (mm³) | C_{RTV}^{b} (%) | (%) |
| Vehicle | 1180 ± 207 | — | — |
| AZD9291, 5 mg/kg | 123 ± 39 | 10.65 | 107.63 |
| Example 2, 5 mg/kg | 230 ± 93 | 19.70 | 96.93 |
| Example 2, 15 mg/kg | 46 ± 32 | 3.93 | 115.76 |
| Example 2, 30 mg/kg | 45 ± 19 | 3.92 | 115.52 |
| Example 6, 5 mg/kg | 203 ± 24 | 17.49 | 99.56 |
| Example 6, 15 mg/kg | 63 ± 30 | 5.34 | 114.04 |
| Example 6, 30 mg/kg | 28 ± 9 | 2.42 | 117.41 |

^{a}Average value, ±SEM, n = 9.
^{b}Relative tumor volumn T_{RTV}/C_{RTV} % = T_{RTV}/C_{RTV} × 100%, RTV = V_{D10}/V_{D0}
^{c}Tumor Growth Inhibition: TGI % = [1-(T_{D10}-T_{D0})/(V_{D10}-V_{D0})] × 100%

Patch Clamp Assay for hERG Inhibition
1. Cells. HEK 293 cell line stably expressing hERG channel (Cat # K1236) was purchased from Invitrogen. The cells are cultured in 85% DMEM, 10% dialyzed FBS, 0.1 mM NEAA, 25 mM HEPES, 100 U/mL Penicillin-Streptomycin and 5 μg/mL Blasticidin and 400 μg/mL Geneticin. Cells are split using TrypLE™ Express about three times a week, and maintained between ˜40% to ˜80% confluence. Before the assay, the cells were onto the coverslips at 5×105 cells/per 6 cm cell culture dish and induced with doxycycline at 1 μg/mL for 48 hours.
2. Solutions. Extracellular solution (in mM): 132 NaCl, 4 KCl, 3 CaCl₂, 0.5 MgCl₂, 11.1 glucose, and 10 HEPES (pH adjusted to 7.35 with NaOH). Intracellular solution (in mM): 140 KCl, 2 MgCl₂, 10 EGTA, 5 MgATP, 10 HEPES (pH adjusted to 7.35 with KOH)
3. Test compounds. Test compounds were initially prepared in DMSO with final concentration of 30 mM as stock solution. The stock solution was further diluted with DMSO to prepare intermediate solution with concentration of 10.0, 3.0, 1.0, and 0.3 mM respectively. Before the experiment, the working solutions were finally prepared by dilution of above described serial solutions in 1000 folds using extracellular solution to reach the final concentration of 30, 10, 3, 1 and 0.3 μM, while the final concentration of DMSO was 0.1% in working solutions.
4. Ion channel current measurement. The cell culture dish was placed it on a microscope stage in a bath chamber, and a desirable cell was located using the ×10 objective. The tip of the electrode was guided to the surface of the cell, and a gigaohm seal was established using gentle suction through the side port of the electrode holder. The C_{fast} cancellation control was used to remove the capacity current in coincidence with the voltage step, and the whole cell configuration was obtained by applying repetitive, brief, strong suction until the membrane patch had ruptured. The membrane potential was set to −60 mV at this point to ensure that hERG channels were closed, and the spikes of capacity current were then cancelled using the C_{slow} cancellation control on the amplifier. The holding potential was set to −90 mV for 1 second, the record current to 50 kHz and the filter to 10 kHz. Leaking current was tested at −80 mV for 500 ms. The hERG current was elicited by depolarizing at +30 mV for 4.8 seconds and then the voltage was taken back to 50 mV for 5.2 seconds to remove the inactivation and observe the deactivating tail current. The maximum amount of tail current size was used to determine hERG current amplitude. The current was recorded for 120 seconds to assess the current stability. Only stable cells with recording parameters above threshold were applied for the drug administrations. Then vehicle control was applied to the cells to establish the baseline. Once the hERG current was found to be stabilized for 3 minutes, test compound was applied. hERG current in the presence of test compound were recorded for approximately 5 minutes to reach steady state and then 5 sweeps were captured. For dose response testing, 5 doses of compound were applied to the cells cumulatively from low to high concentrations. In order to ensure the good performance of cultured cells and operations, the positive control, Dofetilide, with 5 dose concentration was also used to test the same batch of cells.
5. hERG current IC₅₀ determination were done using a five point dose curve. Either Patchmaster or Clampfit software was used to analyze the data, using the expression:

$$Peak Current Inhibition = \frac{( Peak tail {current}_{compound} )}{( 1 - Peak tail {current}_{vehicle} )} \times 100$$

The data was fitted to a sigmoid dose curve using Graphpad Prism 6.0.

TABLE 9
hERG Data

| Run # | Example # | IC₅₀ μM |
|---|---|---|
| 1 | Dofetilide | 0.012 |
| 1 | AZD-9291 | 1.776 |
| 1 | 2 | 6.574 |
| 2 | Dofetilide | 0.010 |
| 2 | AZD-9291 | 2.632 |
| 2 | 3 | 0.858 |
| 2 | 4 | 2.229 |
| 2 | 5 | 3.494 |
| 2 | 6 | 2.55 |
| 2 | 7 | 1.091 |
| 2 | 18 | 1.677 |
| 2 | 19 | 1.101 |
| 2 | 20 | 0.500 |
| 2 | 21 | 3.232 |
| 3 | Dofetilide | 0.013 |
| 3 | 9 | 1.005 |
| 3 | 11 | 5.779 |
| 3 | 12 | 4.082 |

TABLE 10
hERG Data for Comparative Examples

| Run # | Example # | IC₅₀ μM |
|---|---|---|
| 3 | 8 | 2.228 |
| 3 | 13 | 1.652 |

Enzyme assays for IC₅₀ determinations for various enzymes were carried out in accordance with the procedures disclosed herein. In Tables 1-10 Example numbers corresponds to compounds prepared in referenced Example numbers.

The patents and publications listed herein describe the general skill in the art and are hereby incorporated by reference in their entireties for all purposes and to the same extent as if each was specifically and individually indicated to be incorporated by reference. In the case of any conflict between a cited reference and this specification, the specification shall control. In describing embodiments of the present application, specific terminology is employed for the sake of clarity. However, the invention is not intended to be limited to the specific terminology so selected. Nothing in this specification should be considered as limiting the scope of the present invention. All examples presented are representative and non-limiting. The above-described embodiments may be modified or varied, without departing from the invention, as appreciated by those skilled in the art in light of the above teachings. It is therefore to be understood that, within the scope of the claims and their equivalents, the invention may be practiced otherwise than as specifically described.

1. A compound of formula (A) or (B): 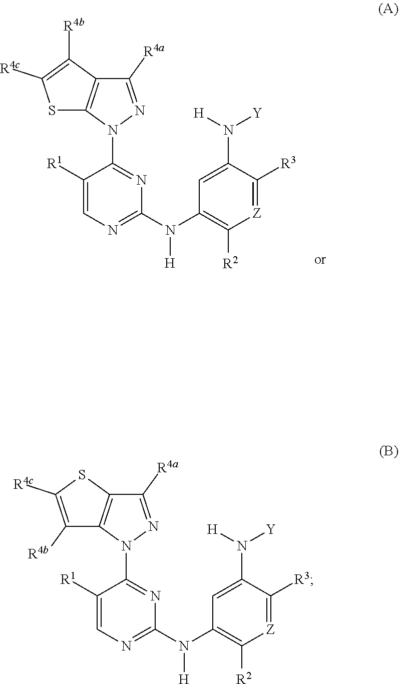
or a stereoisomer or a pharmaceutically acceptable salt, solvate, ester, or prodrug thereof;
wherein,
Z is CH or N;
Y is 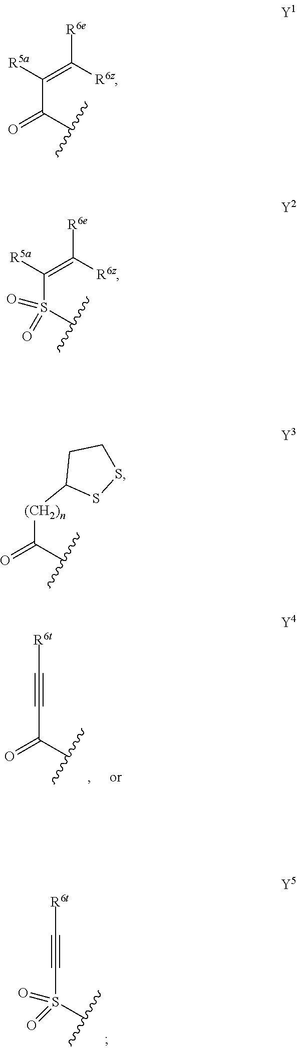
in Y¹ and Y², R^{5a} is H, F, Cl, CF₃, CHF₂, CF₂C₁₋₆ alkyl, CF₂CH₂NR⁸R⁹, CH₂NR⁸R⁹, CN, or C₁₋₆ alkyl;
in Y¹ and Y², R^{6e} is R¹⁰, H, F, aryl, heteroaryl, cycloalkyl, heterocycloalkyl, (CH₂)ₘCHR¹⁰R⁷, CF₂(CH₂)ₘCHR¹⁰R⁷, or C(R¹⁰)₂R⁷;
in Y⁴ and Y⁵, R^{6t} is C₁₋₆ alkyl, C₃₋₆ cycloalkyl, aryl, heteroaryl, heterocycloalkyl, (CH₂)ₘCHR¹⁰R⁷, C(R¹⁰)₂R;
in Y¹ and Y², R^{6z} is H, F, Cl, CF₃, CHF₂, CF₂C₁₋₆ alkyl or C₁₋₆ alkyl; or
alternatively in Y¹ and Y², R^{6e} and R^{6z}, taken together, form ═CR^{6e′}R^{6z′} (allene), wherein R^{6e′} is R¹⁰, H, F, aryl, heteroaryl, cycloalkyl, heterocycloalkyl, (CH₂)ₘCHR¹⁰R⁷, CF₂(CH₂)ₘCHR¹⁰R⁷, or C(R¹⁰)₂R⁷ and wherein, R^{6z′} is H, F, Cl, CF₃, CHF₂, CF₂C₁₋₆ alkyl or C₁₋₆ alkyl; or
alternatively in Y¹ and Y², R^{6e} and R^{6z}, taken together with the sp² carbon atom to which both are attached, form an alicyclic ring of 4 to 7 members wherein one of the ring atoms are optionally replaced by NR⁸, O, S(O)ₓ, S(═O)(═NR⁸), P═O, P(═O)(OR⁸), OP(═O)(OR⁸)O, and the alicyclic ring is optionally substituted with one or more substituents selected from the group consisting of halogen, oxo, OH, OR⁸, and NR⁸R⁹;
R¹ is independently selected from hydrogen, fluoro, chloro, bromo, methyl, ethyl, hydroxyl, methoxy, ethoxy, isopropoxy, cyclopropoxy, —OCF₃, —OCH₂CF₃, —OCH₂CHF₂, ethenyl, ethynyl, —CF₃, —CHF₂, —CHO, —CH₂OH, —CONH₂, —CO₂Me, —CONHMe, —CONMe₂, and cyano;
R² is —OCF₃, —OCHF₂, —OCF₂CF₃, —OCH₂CHF₂, —OCH₂CF₃, cyclopropyl, cyclopropoxy, methoxy, —OCD₃, ethoxy, or isopropoxy;
R³ is —N(R¹⁰)C₂₋₆ alkyl-NR¹⁰R¹⁰, —N(R¹⁰)C₂₋₆ alkyl-R⁷, —O(CH₂)ₚR⁷, —N(R¹⁰)C(═O)(CH₂)ₚR⁷, or R⁷;
each R^{4a}, R^{4b}, and R^{4c} are independently H, cyano, nitro, halo, —C₁₋₆ alkyl, —C₁₋₆ haloalkyl, -carboxy-C₁₋₆ alkyl, —C₁₋₆ hydroxyalkyl, R⁸R⁹N—C₁₋₆ alkyl-, —C₂₋₆ alkenyl, —C₂₋₆ alkynyl, C₁₋₆ acyl-, R⁷—(CH₂)ₚC(═O)—, C₁₋₆ hydroxyalkyl-C(═O)—, carboxy, —C₁₋₆ alkoxycarbonyl, —C(═O)NR⁸R⁹, hydroxyl, —C₁₋₆ alkoxy, —C₁₋₆ acyloxy, —NR⁸R⁹, C₁₋₆ acyl-N(R¹⁰)—, pyrazole, 1,2,3-triazole, tetrazole, (C₁₋₆ alkyl)SO₂—, or R⁷SO₂—;
R⁷ is —OH, —NR⁸R⁹, —O(CH₂)_{q}NR⁸R⁹, C₁₋₆ alkoxy, C₁₋₆ alkoxy-C₁₋₆ alkoxy, C₂₋₆ hydroxyalkoxy, oxetanyl, oxetanyloxy, oxetanylamino, oxolanyl, oxolanyloxy, oxolanylamino, oxanyl oxanyloxy, oxanylamino, oxepanyl, oxepanyloxy, oxepanylamino, azetidinyl, azetidinyloxy, azetidylamino, pyrrolidinyl, pyrolidinyloxy, pyrrolidinylamino, piperidinyl, piperidinyloxy, piperidinylamino, azepanyl, azepanyloxy, azepanylamino, dioxolanyl, dioxanyl, morpholino, thiomorpholino, thiomorpholino-S,S-dioxide, piperazino, dioxepanyl, dioxepanyloxy, dioxepanylamino, oxazepanyl, oxazepanyloxy, oxazepanylamino, diazepanyl, diazepanyloxy, diazepanylamino, (3R)-3-(dimethylamino)pyrrolidin-1-yl, (3S)-3-(dimethylamino)pyrrolidin-1-yl, 3-(dimethylamino)azetidin-1-yl, [2-(dimethylamino)ethyl](methyl)amino, [2-(methylamino)ethyl](methyl)amino, 5-methyl-2,5diazaspiro[3,4]oct-2-yl, (3aR,6aR)-5-methylhexa-hydro-pyrrolo[3,4-b]pyrrol-1(2H)-yl, I-methyl-1,2,3,6-tetrahydropyridin-4-yl, 4-methylpiperizin-1-yl, 4-[2(dimethylamino)-2-oxoethyl]piperazin-1-yl, methyl[2-(4-methylpiperazin-1yl)ethyl]amino, methyl[2-(morpholin-4-yl)ethyl]amino, 1-amino-1,2,3,6tetrahydropyridin-4-yl, 4-[(2S)-2-aminopropanoyl]piperazin-1-yl, all of which may be optionally substituted with OH, OR¹⁰, oxo, halogen, R¹⁰, CH₂OR¹⁰, or CH₂NR⁸R⁹;
R⁸ and R⁹ are each independently H, —CD₃, C₁₋₆ alkyl, C₃₋₆ alkenyl, C₃₋₆ alkynyl, C₃₋₈ cycloalkyl, —(C₁₋₃ alkyl)-(C₃₋₈ cycloalkyl), C₃₋₈ cycloalkenyl, C₁-C₆ acyl, 4-12 membered monocyclic or bicyclic heterocyclyl, 4-12 membered monocyclic or bicyclic heterocyclyl-C₁-C₆ alkyl-, C₆-C₁₂ aryl, 5-12 membered heteroaryl; wherein R⁸ and R⁹ may be further independently substituted with up to three substituents chosen from hydroxyl, C₁₋₆ alkoxy, C₁₋₆ hydroxyalkyl, C₁₋₆ alkoxy-C₁₋₆ alkyl, C₁₋₆ alkoxy-C₁₋₆ alkoxy, C₂₋₆ hydroxyalkoxy, oxo, thiono, cyano or halo; or
alternatively, R⁸ and R⁹, taken together with the N atom to which they are both attached, form a heterocyclic ring of 4-7 members, containing up to one other heteroatom selected from O, S, or NR¹¹, or a heterobicyclic ring of 7-12 members which may be fused, bridged or spiro, and contain up to two other heteroatoms chosen from O, S(O)ₓ, or NR¹¹, and these heterocyclic rings are optionally substituted with up to three substituents chosen from hydroxyl, C₁₋₆ alkoxy, C₁₋₆ hydroxyalkyl, C₁₋₆ alkoxy-C₁₋₆ alkyl, C₁₋₆ alkoxy-C₁₋₆ alkoxy, C₂₋₆ hydroxyalkoxy, oxo, thiono, cyano or halo;
each R¹⁰ is independently H, —CD₃, C₁₋₆ alkyl, C₃₋₆ cycloalkyl, C₂₋₆ hydroxyalkyl, C₁₋₆ alkoxy-C₁₋₆ alkyl or C₂₋₆ alkyl-NR⁸R⁹;
alternatively, two R¹⁰ on the same N atom to which they are both attached, form a heterocyclic ring of 5-6 members, containing up to one other heteroatom selected from O, S, or NR¹¹;
each R¹¹ is independently hydrogen or C₁-C₆ alkyl, which is optionally substituted with up to three substituents selected from hydroxyl, oxo, thiono, cyano or halo;
m is 0, 1, 2, or 3;
n is 1, 2, or 3;
q is 2, 3, or 4;
p is 0, 1, 2, 3, or 4; and
x is 0, 1, or 2.

2. The compound of claim 1, wherein the compound has the structure of formula (A): 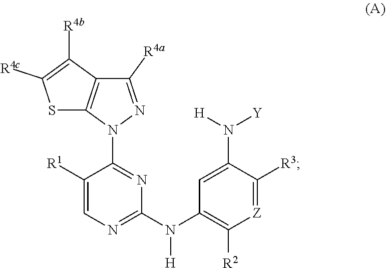
or a stereoisomer or a pharmaceutically acceptable salt, solvate, ester, or prodrug thereof;
wherein,
Z is CH or N;
R¹ is selected from hydrogen, fluoro, chloro, bromo, methyl, CF₃, CHF₂, and cyano;
R² is —OCF₃, —OCHF₂, —OCF₂CF₃, —OCH₂CHF₂, —OCH₂CF₃, cyclopropyl, cyclopropoxy, methoxy, —OCD₃, ethoxy, or isopropoxy;
R³ is N(R¹⁰)C₂₋₆ alkyl-NR¹⁰R¹⁰;
R^{4a}, R^{4b} and R^{4c} are each independently H, cyano, halo, —C₁₋₆ alkyl, —C₁₋₆ haloalkyl, carboxy-C₁₋₆ alkyl, —C₁₋₆ hydroxyalkyl, R⁸R⁹N—C₁₋₆ alkyl-, —C₂₋₆ alkenyl, —C₂₋₆ alkynyl, C₁₋₆ acyl-, R—(CH₂)ₚC(═O)—, C₁₋₆ hydroxyalkyl-C(═O)—, carboxy, —C₁₋₆ alkoxycarbonyl, —C(═O)NR⁸R⁹, hydroxyl, alkoxy, C₁₋₆ acyloxy, —NR⁸R⁹, C₁₋₆ acyl-N(R¹⁰)—, R⁷SO₂—,
R⁷ is OH, NR⁸R⁹, O(CH₂)_{q}NR⁸R⁹, C₁₋₆ alkoxy, or C₂₋₆ hydroxyalkoxy;
R⁸ and R⁹ are independently H, —CD₃, C₁₋₆ alkyl, C₃₋₆ alkenyl, C₃₋₆ alkynyl, C₃₋₈ cycloalkyl, C₃₋₈ cycloalkenyl, C₁-C₆ acyl, 4-12 membered monocyclic or bicyclic heterocyclyl, 4-12 membered monocyclic or bicyclic heterocyclyl-C₁-C₆ alkyl-, C₆-C₁₂ aryl, 5-12 membered heteroaryl; and R⁸ and R⁹ may be further independently substituted with up to three substituents chosen from hydroxyl, C₁₋₆ alkoxy, C₁₋₆ hydroxyalkylC₂₋₆ hydroxyalkoxy, oxo, thiono, cyano or halo; or
alternatively, R⁸ and R⁹, taken together with the N atom to which they are both attached, form a heterocyclic ring of 4-7 members, containing up to one other heteroatom selected from O, S, or NR¹¹, or a heterobicyclic ring of 7-12 members which may be fused, bridged or spiro, and contain up to two other heteroatoms chosen from O, S(O)ₓ, or NR¹¹, and these heterocyclic rings are optionally substituted with up to three substituents chosen from hydroxyl, C₁₋₆ alkoxy, C₁₋₆ hydroxyalkyl, C₁₋₆ alkoxy-C₁₋₆ alkyl, C₁₋₆ alkoxy-C₁₋₆ alkoxy, C₂₋₆ hydroxyalkoxy, oxo, thiono, cyano or halo;
each R¹⁰ is independently H, —CD₃, C₁₋₆ alkyl, C₃₋₆ cycloalkyl, C₂₋₆ hydroxyalkyl, C₁₋₆ alkoxy-C₁₋₆ alkyl or C₂₋₆ alkyl-NR⁸R⁹; or
p is 0, 1, 2, 3, or 4;
q is 2, 3, or 4; and
x is 0, 1, or 2.

3. The compound of claim 1 or 2, wherein R³ is —N(CH₃)CH₂CH₂NR¹⁰R¹⁰.

4. The compound of any one of claims 1-3, wherein R¹⁰ is each independently H, —CD₃, C₁₋₆ alkyl, C₃₋₆ cycloalkyl, or C₂₋₆ hydroxyalkyl.

5. The compound of any one of claims 1-3, wherein R¹⁰ is each independently H, —CD₃, methyl, ethyl, or isopropyl.

6. The compound of any one of claims 1-5, wherein Y is

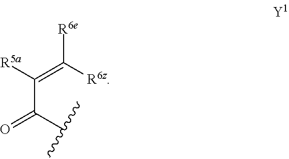

7. The compound of claim 6, wherein R^{5a}, R^{6e}, and R^{6z} are each H.

8. The compound of any one of claims 1-7, wherein R^{4a} is H, —C₁₋₆ alkyl, or —NR⁸R⁹.

9. The compound of claim 8, wherein R⁸ and R⁹ are independently H, —CD₃, or C₁₋₆ alkyl.

10. The compound of any one of claims 1-9, wherein R^{4b} and R^{4c} are each independently H, cyano, F, Cl, Br, —C₁₋₆ alkyl, CF₃, CHF₂, CONH₂ or C(═O)NR⁸R⁹.

11. The compound of any one of claims 1-10, wherein R^{4b} and R^{4c} are each independently H, cyano, F, Cl, Br, CH₃, CF₃, CHF₂, CONH₂ or C(═O)NR⁸R⁹.

12. The compound of claim 1, wherein the compound has the structure of formula (C): 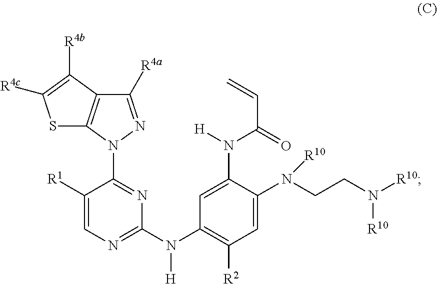
or a stereoisomer or a pharmaceutically acceptable salt, solvate, ester, or prodrug thereof;
wherein,
R¹ is hydrogen, fluoro, chloro, or methyl;
R² is —OCF₃, —OCHF₂, —OCF₂CF₃, —OCH₂CHF₂, —OCH₂CF₃, cyclopropyl, cyclopropoxy, methoxy, —OCD₃, ethoxy, or isopropoxy;
R^{4a} is H or —NR⁸R⁹;
R^{4b} and R^{4c} are each independently H, cyano, F, Cl, Br, CH₃, CF₃, CHF₂, CONH₂, or C(═O)NR⁸R⁹;
R⁸ and R⁹ are each independently H, —CD₃, or C₁₋₆ alkyl; and
each R¹⁰ is independently H, —CD₃, C₁₋₆ alkyl, C₃₋₆ cycloalkyl, or C₂₋₆ hydroxyalkyl.

13. The compound of claim 12, wherein:
R¹ is hydrogen;
R² is methoxy, —OCD₃, ethoxy, or isopropoxy;
R^{4a} is NR⁸R⁹;
R^{4b} is H, or CH₃;
R^{4c} is H, F, Cl, Br, or CH₃;
R⁸ and R⁹ are each independently H, —CD₃, —CH₃, —CH₂CH₃, or —CH(CH₃)₂; and
each R¹⁰ is independently H, —CD₃, —CH₃, —CH₂CH₃, or —CH(CH₃)₂.

14. The compound of claim 1, wherein the compound has the structure of formula (C-I): 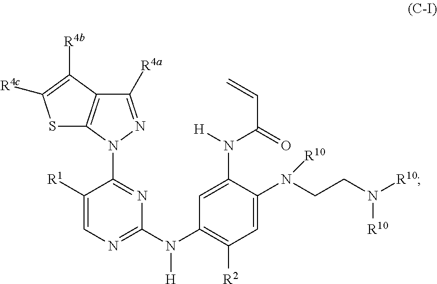
or a stereoisomer or a pharmaceutically acceptable salt, solvate, ester, or prodrug thereof;
wherein,
R¹ is hydrogen, fluoro, chloro, or methyl;
R² is —OCF₃, —OCHF₂, —OCF₂CF₃, —OCH₂CHF₂, —OCH₂CF₃, cyclopropyl, cyclopropoxy, methoxy, —OCD₃, ethoxy, or isopropoxy;
R^{4a} is H or —NR⁸R⁹;
R^{4b} and R^{4c} are each independently H, cyano, F, Cl, Br, —C₁₋₆ alkyl, —CF₃, —CHF₂, —CONH₂, or —C(═O)NR⁸R⁹;
R⁸ and R⁹ are each independently H, —CD₃, or —C₁₋₆ alkyl; and
each R¹⁰ is independently H, —CD₃, —C₁₋₆ alkyl, —C₃₋₆ cycloalkyl, or —C₂₋₆ hydroxyalkyl.

15. The compound of claim 14, wherein:
R¹ is hydrogen;
R² is methoxy, —OCD₃, ethoxy, or isopropoxy;
R^{4a} is NR⁸R⁹;
R^{4b} is H, or CH₃;
R^{4c} is H, F, Cl, Br, —CF₃, —CH₃, —CH₂CH₃ or —CH(CH₃)₂;
R⁸ and R⁹ are each independently H, —CD₃, —CH₃, —CH₂CH₃, or —CH(CH₃)₂; and
each R¹⁰ is independently H, —CD₃, —CH₃, —CH₂CH₃, or —CH(CH₃)₂.

16. The compound of any one of claims 1-15, wherein the compound is: 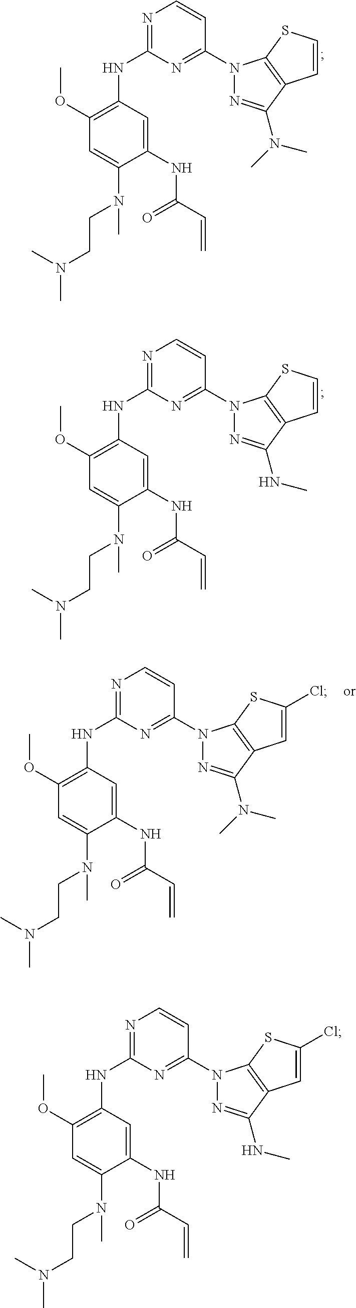 [SMILES: C=CC(=O)Nc1cc(Nc2nccc(-n3nc(N(C)C)c4ccsc43)n2)c(OC)cc1N(C)CCN(C)C ; C=CC(=O)Nc1cc(Nc2nccc(-n3nc(NC)c4ccsc43)n2)c(OC)cc1N(C)CCN(C)C ; C=CC(=O)Nc1cc(Nc2nccc(-n3nc(N(C)C)c4cc(Cl)sc43)n2)c(OC)cc1N(C)CCN(C)C ; C=CC(=O)Nc1cc(Nc2nccc(-n3nc(NC)c4cc(Cl)sc43)n2)c(OC)cc1N(C)CCN(C)C]
or a stereoisomer or a pharmaceutically acceptable salt, solvate, ester, or prodrug thereof.

17. The compound of any one of claims 1-15, wherein the compound is: 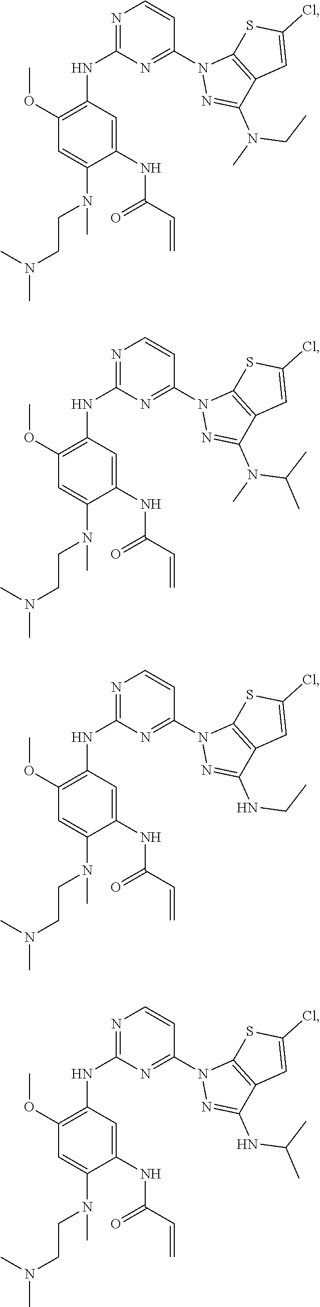 [SMILES: C=CC(=O)Nc1cc(Nc2nccc(-n3nc(N(C)CC)c4cc(Cl)sc43)n2)c(OC)cc1N(C)CCN(C)C ; C=CC(=O)Nc1cc(Nc2nccc(-n3nc(N(C)C(C)C)c4cc(Cl)sc43)n2)c(OC)cc1N(C)CCN(C)C ; C=CC(=O)Nc1cc(Nc2nccc(-n3nc(NCC)c4cc(Cl)sc43)n2)c(OC)cc1N(C)CCN(C)C ; C=CC(=O)Nc1cc(Nc2nccc(-n3nc(NC(C)C)c4cc(Cl)sc43)n2)c(OC)cc1N(C)CCN(C)C] 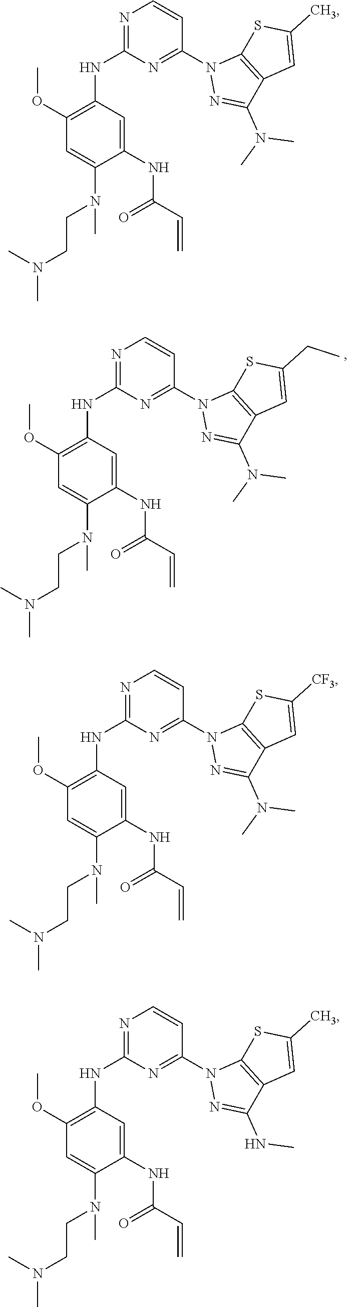 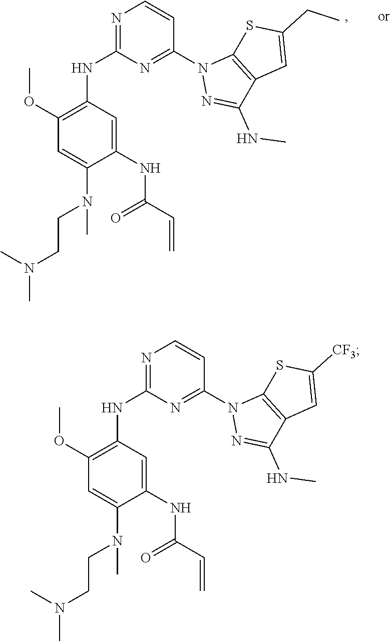
or a stereoisomer or a pharmaceutically acceptable salt, solvate, ester, or prodrug thereof.

18. A compound of formula (D): 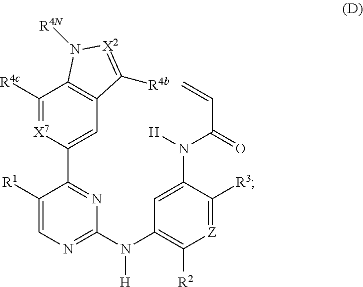
or a stereoisomer or a pharmaceutically acceptable salt, solvate, ester, or prodrug thereof;
wherein,
Z is CH or N;
X² and X⁷ are each CH, CR⁴, or N;
R¹ is hydrogen, fluoro, chloro, bromo, methyl, ethyl, hydroxyl, methoxy, ethoxy, isopropoxy, cyclopropoxy, —OCF₃, —OCH₂CF₃, —OCH₂CHF₂, ethenyl, ethynyl, CF₃, CHF₂, CHO, CH₂OH, CONH₂, CO₂Me, CONHMe, CONMe₂, or cyano;
R² is —OCF₃, —OCHF₂, —OCF₂CF₃, —OCH₂CHF₂, —OCH₂CF₃, cyclopropyl, cyclopropoxy, methoxy, —OCD₃, ethoxy, or isopropoxy;
R³ is N(R¹⁰)C₂₋₆ alkyl-NR¹⁰R¹⁰;
each R⁴ is independently H, cyano, halo, —C₁₋₆ alkyl, —C₁₋₆ haloalkyl, carboxy-C₁₋₆ alkyl, —C₁₋₆ hydroxyalkyl, R⁸R⁹N—C₁₋₆ alkyl-, —C₂₋₆ alkenyl, —C₂₋₆ alkynyl, C₁₋₆ acyl-, R⁷—(CH₂)ₚC(═O)—, C₁₋₆ hydroxyalkyl-C(═O)—, carboxy, —C₁₋₆ alkoxycarbonyl, —C(═O)NR⁸R⁹, hydroxyl, alkoxy, C₁₋₆ acyloxy, —NR⁸R⁹, C₁₋₆ acyl-N(R¹⁰)—, or R⁷SO₂—; and
R⁸ and R⁹ are independently H, —CD₃, C₁₋₆ alkyl, C₃₋₆ alkenyl, C₃₋₆ alkynyl, C₃₋₈ cycloalkyl, C₃₋₈ cycloalkenyl, C₁-C₆ acyl, 4-12 membered monocyclic or bicyclic heterocyclyl, 4-12 membered monocyclic or bicyclic heterocyclyl-C₁-C₆ alkyl-, C₆-C₁₂ aryl, 5-12 membered heteroaryl; and R⁸ and R⁹ may be further independently substituted with up to three substituents chosen from hydroxyl, C₁₋₆ alkoxy, C₁₋₆ hydroxyalkylC₂₋₆ hydroxyalkoxy, oxo, thiono, cyano or halo; or
alternatively, R⁸ and R⁹, taken together with the N atom to which they are both attached, form a heterocyclic ring of 4-7 members, containing up to one other heteroatom selected from O, S, or NR¹¹, or a heterobicyclic ring of 7-12 members which may be fused, bridged or spiro, and contain up to two other heteroatoms chosen from O, S(O)ₓ, or NR¹¹, and these heterocyclic rings are optionally substituted with up to three substituents chosen from hydroxyl, C₁₋₆ alkoxy, C₁₋₆ hydroxyalkyl, C₁₋₆ alkoxy-C₁₋₆ alkyl, C₁₋₆ alkoxy-C₁₋₆ alkoxy, C₂₋₆ hydroxyalkoxy, oxo, thiono, cyano or halo;
R^{4b} is H, halo, —C₁₋₆ alkyl, or —C₁₋₆ haloalkyl;
R^{4c} is cyano, C₁₋₆ acyl-, —C(═O)NR⁸R⁹, hydroxyl, alkoxy, or F;
R^{4N} is H, —CD₃, or —C₁₋₆ alkyl;
R⁷ is OH, NR⁸R⁹, —O(CH₂)_{q}NR⁸R⁹, C₁₋₆ alkoxy, or C₂₋₆ hydroxyalkoxy;
each R¹⁰ is independently H, —CD₃, C₁₋₆ alkyl, C₃₋₆ cycloalkyl, C₂₋₆ hydroxyalkyl, C₁₋₆ alkoxy-C₁₋₆ alkyl or C₂₋₆ alkyl-NR⁸R⁹;
p=0, 1, 2, 3, or 4;
q=2, 3, or 4; and
x=0, 1, or 2.

19. A compound of formula (D-I): 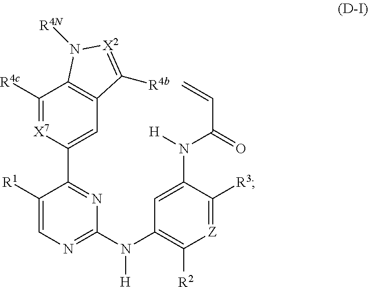
or a stereoisomer or a pharmaceutically acceptable salt, solvate, N-oxide, ester, or prodrug thereof;
wherein,
Z is CH or N;
X² and X⁷ are each CH, CR⁴, or N;
R¹ is hydrogen, fluoro, chloro, bromo, methyl, ethyl, hydroxyl, methoxy, ethoxy, isopropoxy, cyclopropoxy, —OCF₃, —OCH₂CF₃, —OCH₂CHF₂, ethenyl, ethynyl, CF₃, CHF₂, CHO, CH₂OH, CONH₂, CO₂Me, CONHMe, CONMe₂, or cyano;
R² is —OCF₃, —OCHF₂, —OCF₂CF₃, —OCH₂CHF₂, —OCH₂CF₃, cyclopropyl, cyclopropoxy, methoxy, —OCD₃, ethoxy, or isopropoxy;
R³ is —N(R¹⁰)(C₂₋₆ alkyl)-NR¹⁰R¹⁰ or —N(R¹⁰)(C₃₋₁₀ cycloalkylalkyl)-NR¹⁰R¹⁰;
each R⁴ is independently H, cyano, halo, —C₁₋₆ alkyl, —C₁₋₆ haloalkyl, carboxy-C₁₋₆ alkyl, —C₁₋₆ hydroxyalkyl, R⁸R⁹N—C₁₋₆ alkyl-, —C₂₋₆ alkenyl, —C₂₋₆ alkynyl, C₁₋₆ acyl-, R⁷—(CH₂)ₚC(═O)—, C₁₋₆ hydroxyalkyl-C(═O)—, carboxy, —C₁₋₆ alkoxycarbonyl, —C(═O)NR⁸R⁹, hydroxyl, alkoxy, C₁₋₆ acyloxy, —NR⁸R⁹, C₁₋₆ acyl-N(R¹⁰)—, or R⁷SO₂—; and
R⁸ and R⁹ are independently H, —CD₃, C₁₋₆ alkyl, C₃₋₆ alkenyl, C₃₋₆ alkynyl, C₃₋₈ cycloalkyl, C₃₋₈ cycloalkenyl, C₁-C₆ acyl, 4-12 membered monocyclic or bicyclic heterocyclyl, 4-12 membered monocyclic or bicyclic heterocyclyl-C₁-C₆ alkyl-, C₆-C₁₂ aryl, 5-12 membered heteroaryl; and R⁸ and R⁹ may be further independently substituted with up to three substituents chosen from hydroxyl, C₁₋₆ alkoxy, C₁₋₆ hydroxyalkylC₂₋₆ hydroxyalkoxy, oxo, thiono, cyano or halo; or
alternatively, R⁸ and R⁹, taken together with the N atom to which they are both attached, form a heterocyclic ring of 4-7 members, containing up to one other heteroatom selected from O, S, or NR¹¹, or a heterobicyclic ring of 7-12 members which may be fused, bridged or spiro, and contain up to two other heteroatoms chosen from O, S(O)ₓ, or NR¹¹, and these heterocyclic rings are optionally substituted with up to three substituents chosen from hydroxyl, C₁₋₆ alkoxy, C₁₋₆ hydroxyalkyl, C₁₋₆ alkoxy-C₁₋₆ alkyl, C₁₋₆ alkoxy-C₁₋₆ alkoxy, C₂₋₆ hydroxyalkoxy, oxo, thiono, cyano or halo;
R^{4b} is H, halo, —C₁₋₆ alkyl, or —C₁₋₆ haloalkyl;
R^{4c} is H, cyano, hydroxyl, alkoxy, —C₁₋₆ alkyl, or —C₁₋₆ haloalkyl, Cl, or F, provided that when R^{4c} is H, R^{4b} is halo, —C₁₋₆ alkyl, or —C₁₋₆ haloalkyl;
R^{4N} is H, —CD₃, or —C₁₋₆ alkyl;
R⁷ is OH, NR⁸R⁹, —O(CH₂)_{q}NR⁸R⁹, C₁₋₆ alkoxy, or C₂₋₆ hydroxyalkoxy;
each R¹⁰ is independently H, —CD₃, C₁₋₆ alkyl, C₃₋₆ cycloalkyl, C₂₋₆ hydroxyalkyl, C₁₋₆ alkoxy-C₁₋₆ alkyl or C₂₋₆ alkyl-NR⁸R⁹; or
alternatively, two R¹⁰ on the same N atom, taken together form a heterocyclic ring of 3-7 members, optionally substituted with up to three substituents chosen from hydroxyl, C₁₋₆ alkoxy, C₁₋₆ hydroxyalkyl, C₁₋₆ alkoxy-C₁₋₆ alkyl, C₁₋₆ alkoxy-C₁₋₆ alkoxy, C₂₋₆ hydroxyalkoxy, oxo, thiono, cyano or halo;
p=0, 1, 2, 3, or 4;
q=2, 3, or 4; and
x=0, 1, or 2.

20. A compound of formula (E): 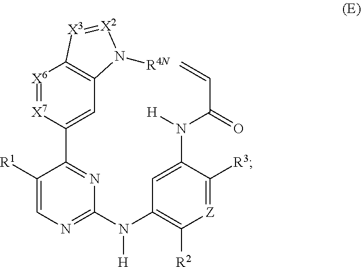
or a stereoisomer or a pharmaceutically acceptable salt, solvate, ester, or prodrug thereof;
wherein,
Z is CH or N;
X², X³, X⁶ and X⁷ are each CH, CR⁴, or N;
R¹ is hydrogen, fluoro, chloro, bromo, methyl, ethyl, hydroxyl, methoxy, ethoxy, isopropoxy, cyclopropoxy, —OCF₃, —OCH₂CF₃, —OCH₂CHF₂, ethenyl, ethynyl, CF₃, CHF₂, CHO, CH₂OH, CONH₂, CO₂Me, CONHMe, CONMe₂, or cyano;
R² is —OCF₃, —OCHF₂, —OCF₂CF₃, —OCH₂CHF₂, —OCH₂CF₃, cyclopropyl, cyclopropoxy, methoxy, —OCD₃, ethoxy, or isopropoxy;
R³ is N(R¹⁰)C₂₋₆ alkyl-NR¹⁰R¹⁰;
each R⁴ is independently H, cyano, halo, —C₁₋₆ alkyl, —C₁₋₆ haloalkyl, carboxy-C₁₋₆ alkyl, —C₁₋₆ hydroxyalkyl, R⁸R⁹N—C₁₋₆ alkyl-, —C₂₋₆ alkenyl, —C₂₋₆ alkynyl, C₁₋₆ acyl-, R⁷—(CH₂)ₚC(═O)—, C₁₋₆ hydroxyalkyl-C(═O)—, carboxy, —C₁₋₆ alkoxycarbonyl, —C(═O)NR⁸R⁹, hydroxyl, alkoxy, C₁₋₆ acyloxy, —NR⁸R⁹, C₁₋₆ acyl-N(R¹⁰)—, or R⁷SO₂—; and
R⁸ and R⁹ are independently H, —CD₃, C₁₋₆ alkyl, C₃₋₆ alkenyl, C₃₋₆ alkynyl, C₃₋₈ cycloalkyl, C₃₋₈ cycloalkenyl, C₁-C₆ acyl, 4-12 membered monocyclic or bicyclic heterocyclyl, 4-12 membered monocyclic or bicyclic heterocyclyl-C₁-C₆ alkyl-, C₆-C₁₂ aryl, 5-12 membered heteroaryl; and R⁸ and R⁹ may be further independently substituted with up to three substituents chosen from hydroxyl, C₁₋₆ alkoxy, C₁₋₆ hydroxyalkylC₂₋₆ hydroxyalkoxy, oxo, thiono, cyano or halo; or
alternatively, R⁸ and R⁹, taken together with the N atom to which they are both attached, form a heterocyclic ring of 4-7 members, containing up to one other heteroatom selected from O, S, or NR¹¹, or a heterobicyclic ring of 7-12 members which may be fused, bridged or spiro, and contain up to two other heteroatoms chosen from O, S(O)ₓ, or NR¹¹, and these heterocyclic rings are optionally substituted with up to three substituents chosen from hydroxyl, C₁₋₆ alkoxy, C₁₋₆ hydroxyalkyl, C₁₋₆ alkoxy-C₁₋₆ alkyl, C₁₋₆ alkoxy-C₁₋₆ alkoxy, C₂₋₆ hydroxyalkoxy, oxo, thiono, cyano or halo;
R^{4N} is H, —CD₃, or —C₁₋₆ alkyl;
R⁷ is OH, NR⁸R⁹, —O(CH₂)_{q}NR⁸R⁹, C₁₋₆ alkoxy, or C₂₋₆ hydroxyalkoxy;
each R¹⁰ is independently H, —CD₃, C₁₋₆ alkyl, C₃₋₆ cycloalkyl, C₂₋₆ hydroxyalkyl, C₁₋₆ alkoxy-C₁₋₆ alkyl or C₂₋₆ alkyl-NR⁸R⁹;
p=0, 1, 2, 3, or 4;
q=2, 3, or 4; and
x=0, 1, or 2.

21. A compound of formula (F) or (G): 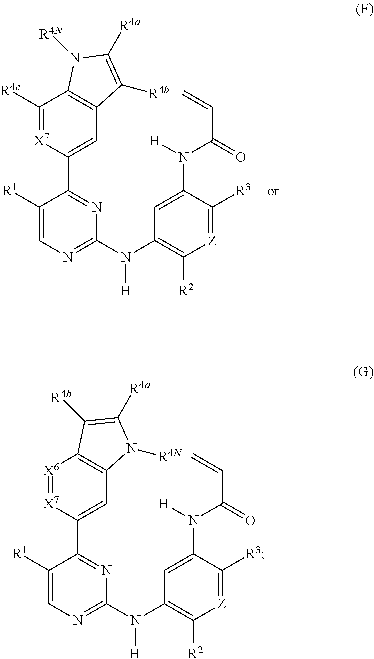
or a stereoisomer or a pharmaceutically acceptable salt, solvate, ester, or prodrug thereof;
wherein,
Z is CH or N;
X⁶ and X⁷ are each CH, CR⁴, or N;
R¹ is independently selected from hydrogen, fluoro, chloro, bromo, methyl, ethyl, hydroxyl, methoxy, ethoxy, isopropoxy, cyclopropoxy, —OCF₃, —OCH₂CF₃, —OCH₂CHF₂, ethenyl, ethynyl, CF₃, CHF₂, CHO, CH₂OH, CONH₂, CO₂Me, CONHMe, CONMe₂, and cyano;
R² is —OCF₃, —OCHF₂, —OCF₂CF₃, —OCH₂CHF₂, —OCH₂CF₃, cyclopropyl, cyclopropoxy, methoxy, —OCD₃, ethoxy, or isopropoxy;
R³ is N(R¹⁰)C₂₋₆ alkyl-NR¹⁰R¹⁰;
each R⁴ is independently H, cyano, halo, —C₁₋₆ alkyl, —C₁₋₆ haloalkyl, carboxy-C₁₋₆ alkyl, —C₁₋₆ hydroxyalkyl, R⁸R⁹N—C₁₋₆ alkyl-, —C₂₋₆ alkenyl, —C₂₋₆ alkynyl, C₁₋₆ acyl-, R⁷—(CH₂)ₚC(═O)—, C₁₋₆ hydroxyalkyl-C(═O)—, carboxy, —C₁₋₆ alkoxycarbonyl, —C(═O)NR⁸R⁹, hydroxyl, alkoxy, C₁₋₆ acyloxy, —NR⁸R⁹, C₁₋₆ acyl-N(R¹⁰)—, R⁷SO₂—, R^{4a} and R^{4b} are each independently H, halo, —C₁₋₆ alkyl, or —C₁₋₆ haloalkyl;
R^{4c} is cyano, C₁₋₆ acyl-, —C(═O)NR⁸R⁹, hydroxyl, alkoxy, or F;
R^{4N} is H, —CD₃, —C₁₋₆ alkyl, or —C₁₋₆ haloalkyl;
R⁷ is OH, NR⁸R⁹, O(CH₂)_{q}NR⁸R⁹, C₁₋₆ alkoxy, or C₂₋₆ hydroxyalkoxy;
R⁸ and R⁹ are independently H, —CD₃, C₁₋₆ alkyl, C₃₋₆ alkenyl, C₃₋₆ alkynyl, C₃₋₈ cycloalkyl, C₃₋₈ cycloalkenyl, C₁-C₆ acyl, 4-12 membered monocyclic or bicyclic heterocyclyl, 4-12 membered monocyclic or bicyclic heterocyclyl-C₁-C₆ alkyl-, C₆-C₁₂ aryl, 5-12 membered heteroaryl; and R⁸ and R⁹ may be further independently substituted with up to three substituents chosen from hydroxyl, C₁₋₆ alkoxy, C₁₋₆ hydroxyalkylC₂₋₆ hydroxyalkoxy, oxo, thiono, cyano or halo; or
each R¹⁰ is independently H, —CD₃, C₁₋₆ alkyl, C₃₋₆ cycloalkyl, C₂₋₆ hydroxyalkyl, C₁₋₆ alkoxy-C₁₋₆ alkyl or C₂₋₆ alkyl-NR⁸R⁹; or
p=0, 1, 2, 3, or 4; and
q=2, 3, or 4.

22. The compound of any one of claims 18-21, wherein the compound is not: 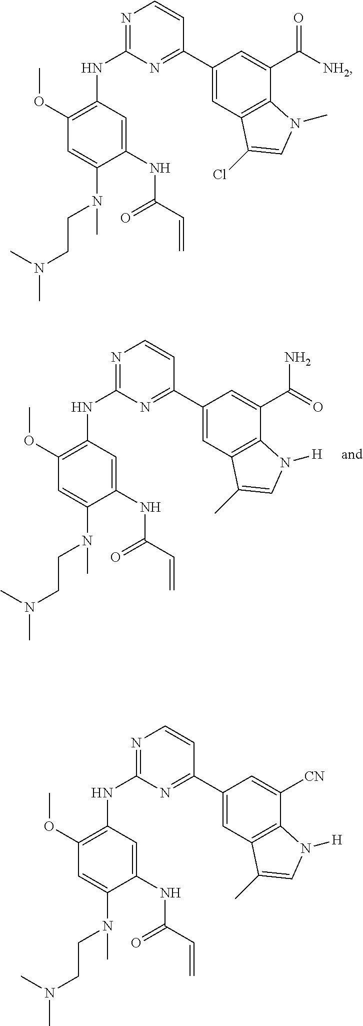 [SMILES: C=CC(=O)Nc1cc(Nc2nccc(-c3cc(C(N)=O)c4c(c3)c(Cl)cn4C)n2)c(OC)cc1N(C)CCN(C)C ; C=CC(=O)Nc1cc(Nc2nccc(-c3cc(C(N)=O)c4[nH]cc(C)c4c3)n2)c(OC)cc1N(C)CCN(C)C ; C=CC(=O)Nc1cc(Nc2nccc(-c3cc(C#N)c4[nH]cc(C)c4c3)n2)c(OC)cc1N(C)CCN(C)C]
or a stereoisomer or a pharmaceutically acceptable salt, solvate, ester, or prodrug thereof.

23. The compound of any one of claims 18-21, wherein the compound is: 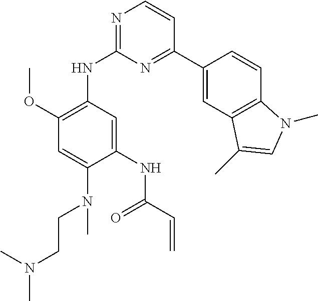
or a stereoisomer or a pharmaceutically acceptable salt, solvate, ester, or prodrug thereof.

24. The compound of claim 19, wherein
X² is CH or CR⁴;
R⁴ is methyl, ethyl, or isopropyl;
R^{4c} is cyano, —CF₃, Cl, or F;
R^{4N} is —CD₃, methyl, ethyl, or isopropyl; and
R^{4b} is H, halo, methyl, ethyl, or isopropyl.

25. The compound of claim 19 or 24, wherein the compound is: 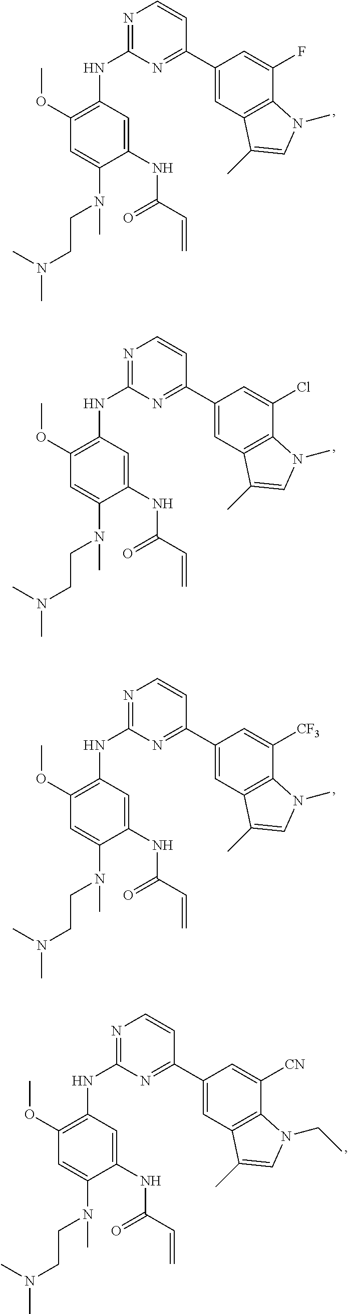 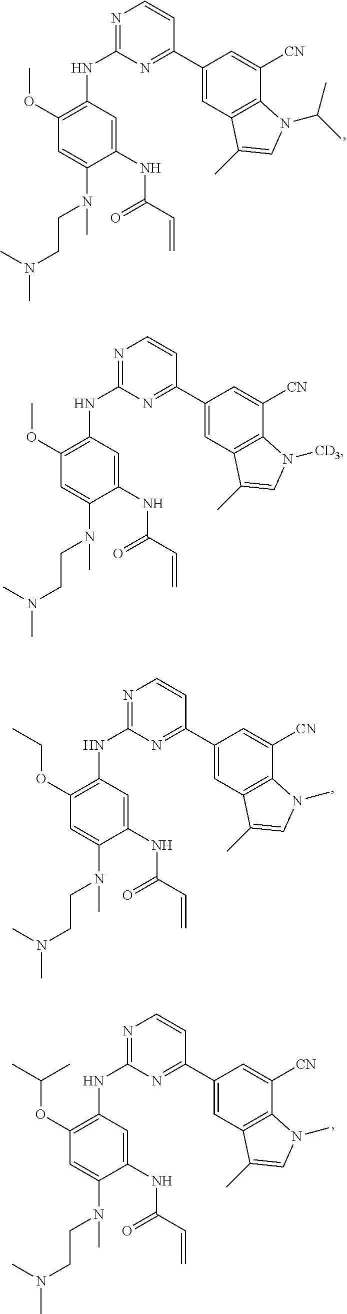 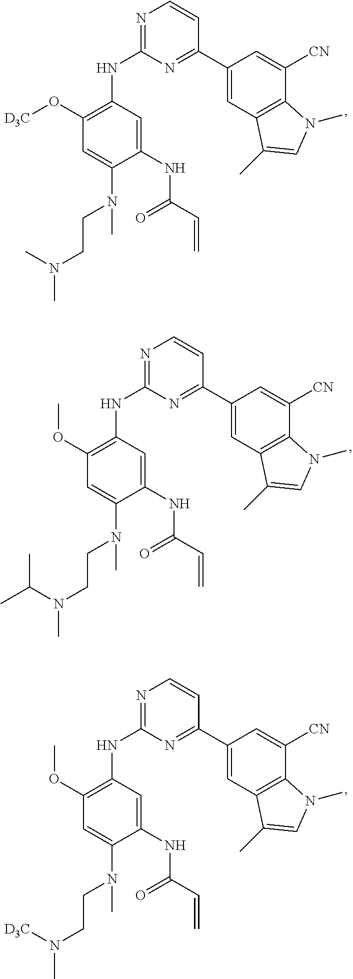 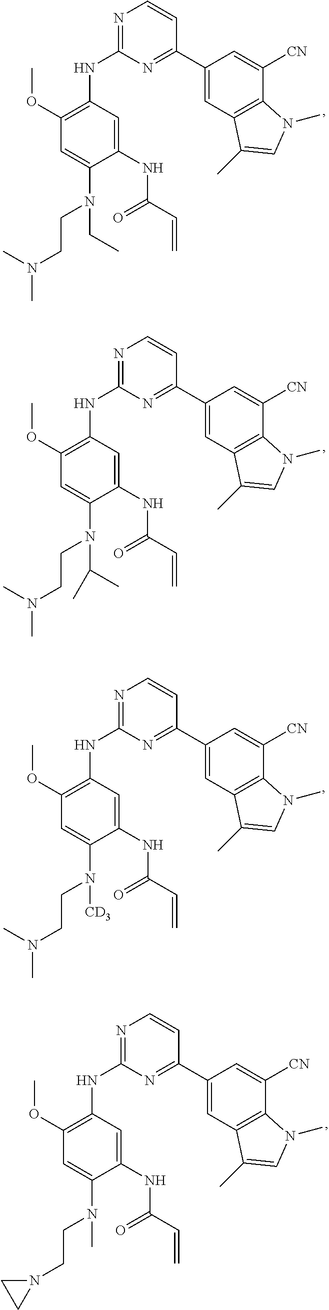 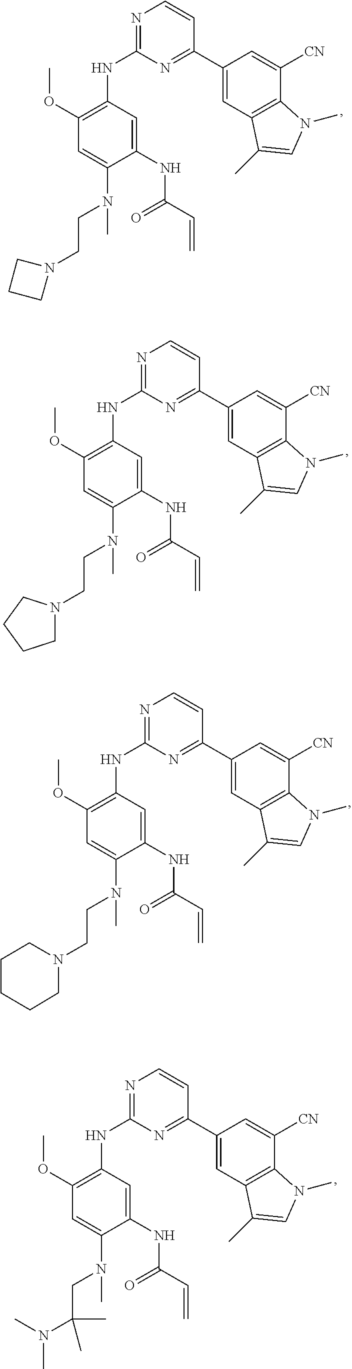 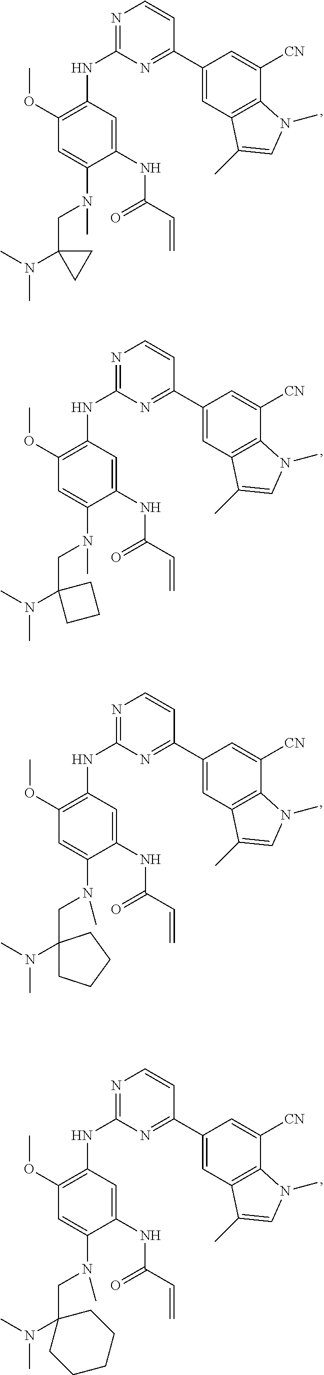 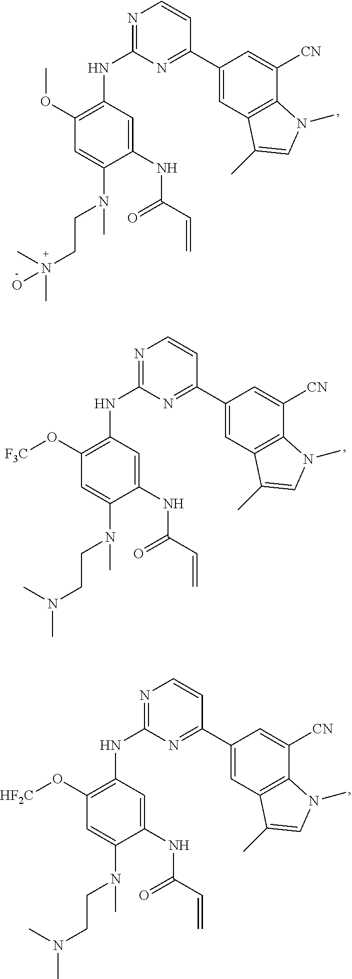 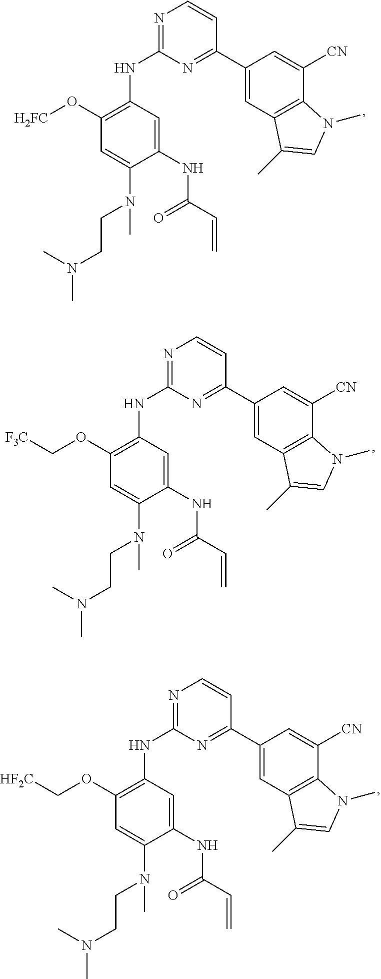 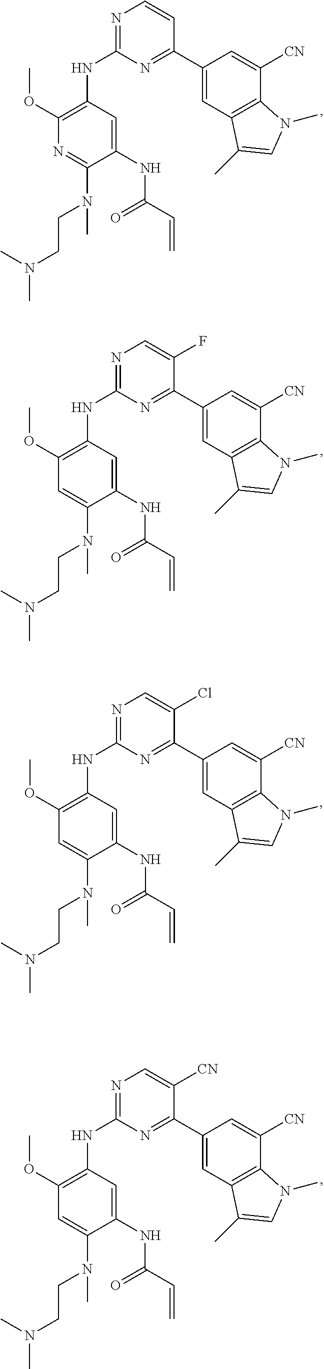 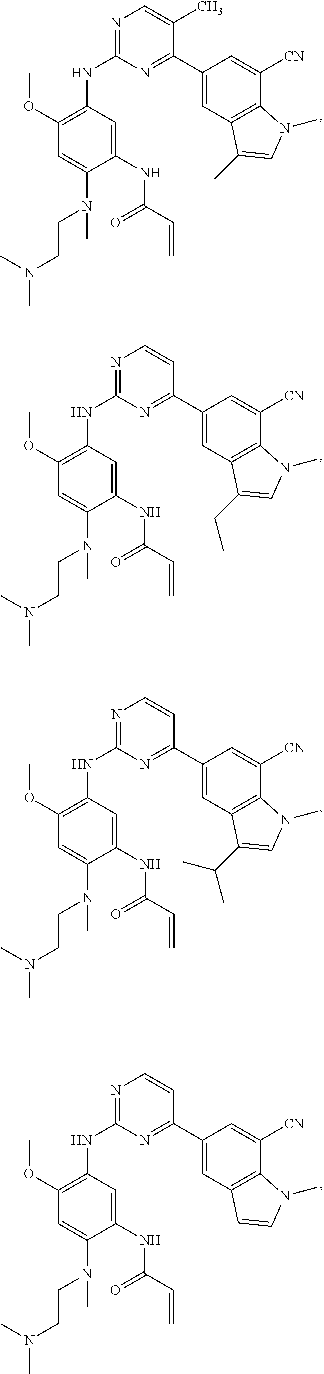 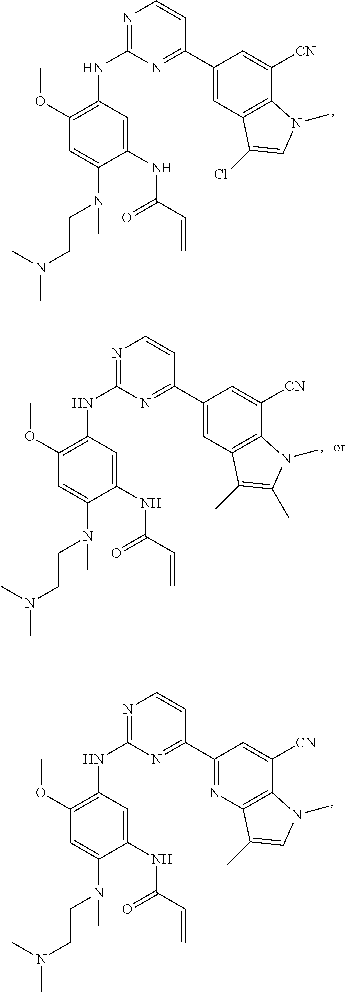
or a stereosomer or a pharmaceutically acceptable salt, solvate, ester, or prodrug thereof.

26. The compound of claim 19, wherein
X² is N;
R^{4c} is cyano, —CF₃, Cl, or F;
R^{4N} is —CD₃, methy-CF₃, Cl, or isopropyl; and
R^{4b} is H, halo, methyl, ethyl, or isopropyl.

27. The compound of claim 26, wherein the compound is 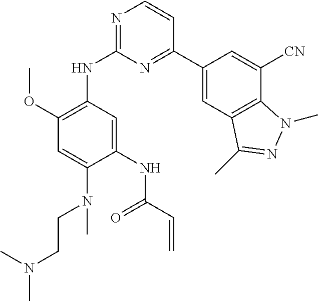
or a stereoisomer or a pharmaceutically acceptable salt, solvate, ester, or prodrug thereof.

28. A compound of formula (E-I): 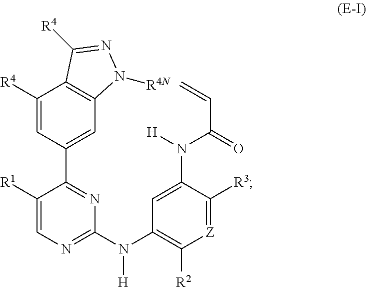
or a stereoisomer or a pharmaceutically acceptable salt, solvate, ester, or prodrug thereof;
wherein,
Z is CH or N;
R¹ is hydrogen, fluoro, chloro, bromo, methyl, ethyl, hydroxyl, methoxy, ethoxy, isopropoxy, cyclopropoxy, —OCF₃, —OCH₂CF₃, —OCH₂CHF₂, ethenyl, ethynyl, CF₃, CHF₂, CHO, CH₂OH, CONH₂, CO₂Me, CONHMe, CONMe₂, or cyano;
R² is —OCF₃, —OCHF₂, —OCF₂CF₃, —OCH₂CHF₂, —OCH₂CF₃, cyclopropyl, cyclopropoxy, methoxy, —OCD₃, ethoxy, or isopropoxy;
R³ is N(R¹⁰)C₂₋₆ alkyl-NR¹⁰R¹⁰ or —N(R¹⁰)(C₃₋₁₀ cycloalkylalkyl)-NR¹⁰R¹⁰;
each R⁴ is independently H, cyano, halo, —C₁₋₆ alkyl, —C₁₋₆ haloalkyl, carboxy-C₁₋₆ alkyl, —C₁₋₆ hydroxyalkyl, R⁸R⁹N—C₁₋₆ alkyl-, —C₂₋₆ alkenyl, —C₂₋₆ alkynyl, C₁₋₆ acyl-, R⁷—(CH₂)ₚC(═O)—, C₁₋₆ hydroxyalkyl-C(═O)—, carboxy, —C₁₋₆ alkoxycarbonyl, —C(═O)NR⁸R⁹, hydroxyl, alkoxy, C₁₋₆ acyloxy, —NR⁸R⁹, C₁₋₆ acyl-N(R¹⁰)—, or R⁷SO₂—; and
R⁸ and R⁹ are independently H, —CD₃, C₁₋₆ alkyl, C₃₋₆ alkenyl, C₃₋₆ alkynyl, C₃₋₈ cycloalkyl, C₃₋₈ cycloalkenyl, C₁-C₆ acyl, 4-12 membered monocyclic or bicyclic heterocyclyl, 4-12 membered monocyclic or bicyclic heterocyclyl-C₁-C₆ alkyl-, C₆-C₁₂ aryl, 5-12 membered heteroaryl; and R⁸ and R⁹ may be further independently substituted with up to three substituents chosen from hydroxyl, C₁₋₆ alkoxy, C₁₋₆ hydroxyalkylC₂₋₆ hydroxyalkoxy, oxo, thiono, cyano or halo; or
alternatively, R⁸ and R⁹, taken together with the N atom to which they are both attached, form a heterocyclic ring of 4-7 members, containing up to one other heteroatom selected from O, S, or NR¹¹, or a heterobicyclic ring of 7-12 members which may be fused, bridged or spiro, and contain up to two other heteroatoms chosen from O, S(O)ₓ, or NR¹¹, and these heterocyclic rings are optionally substituted with up to three substituents chosen from hydroxyl, C₁₋₆ alkoxy, C₁₋₆ hydroxyalkyl, C₁₋₆ alkoxy-C₁₋₆ alkyl, C₁₋₆ alkoxy-C₁₋₆ alkoxy, C₂₋₆ hydroxyalkoxy, oxo, thiono, cyano or halo;
R^{4N} is H, —CD₃, or —C₁₋₆ alkyl;
R⁷ is OH, —NR⁸R⁹, —O(CH₂)_{q}NR⁸R⁹, C₁₋₆ alkoxy, or C₂₋₆ hydroxyalkoxy;
each R¹⁰ is independently H, —CD₃, C₁₋₆ alkyl, C₃₋₆ cycloalkyl, C₂₋₆ hydroxyalkyl, C₁₋₆ alkoxy-C₁₋₆ alkyl or C₂₋₆ alkyl-NR⁸R⁹;
alternatively, two R¹⁰ on the same N atom, taken together form a heterocyclic ring of 3-7 members, optionally substituted with up to three substituents chosen from hydroxyl, C₁₋₆ alkoxy, C₁₋₆ hydroxyalkyl, C₁₋₆ alkoxy-C₁₋₆ alkyl, C₁₋₆ alkoxy-C₁₋₆ alkoxy, C₂₋₆ hydroxyalkoxy, oxo, thiono, cyano or halo;
p=0, 1, 2, 3, or 4;
q=2, 3, or 4; and
x=0, 1, or 2.

29. The compound of claim 28, wherein
R³ is N(R¹⁰)C₂₋₆ alkyl-NR¹⁰R¹⁰ or —N(R¹⁰)(C₃₋₁₀ cycloalkylalkyl)-NR¹⁰R¹⁰;
each R⁴ is independently H, cyano, halo, —C₁₋₆ alkyl, or —C₁₋₆ haloalkyl; and
R^{4N} is H, —CD₃, or —C₁₋₆ alkyl; and
each R¹⁰ is independently H, —CD₃, or —C₁₋₆ alkyl.

30. The compound of claim 28 or 29, wherein the compound is 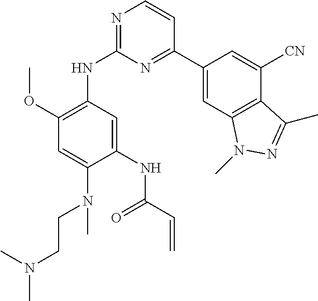
or a stereoisomer or a pharmaceutically acceptable salt, solvate, ester, or prodrug thereof.

31. The compound of claim 18, wherein the compound has the structure of formula (H) 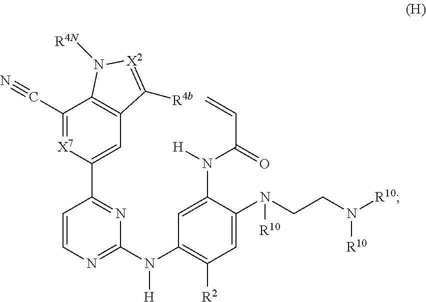
or a stereoisomer or a pharmaceutically acceptable salt, solvate, ester, or prodrug thereof;
wherein,
X⁷ is CH or N;
X² is independently CH, CCH₃, or N;
R² is methoxy, —OCD₃, ethoxy, or isopropoxy;
R^{4b} is H, F, Cl, or CH₃;
R^{4N} is H, —CD₃, CH₃, Et, or CH(CH₃)₂; and
each R¹⁰ is independently H, —CD₃, —CH₃, —CH₂CH₃, or —CH(CH₃)₂.

32. The compound of claim 31, wherein
X⁷ is CH or N;
X² is independently CH or CCH₃;
R² is methoxy, —OCD₃, ethoxy, or isopropoxy;
R^{4b} is H, F, Cl, or CH₃;
R^{4N} is H, —CD₃, CH₃, Et, or CH(CH₃)₂; and
each R¹⁰ is independently H, —CD₃, —CH₃, —CH₂CH₃, or —CH(CH₃)₂.

33. The compound of claim 31 or 32, wherein the compound is: 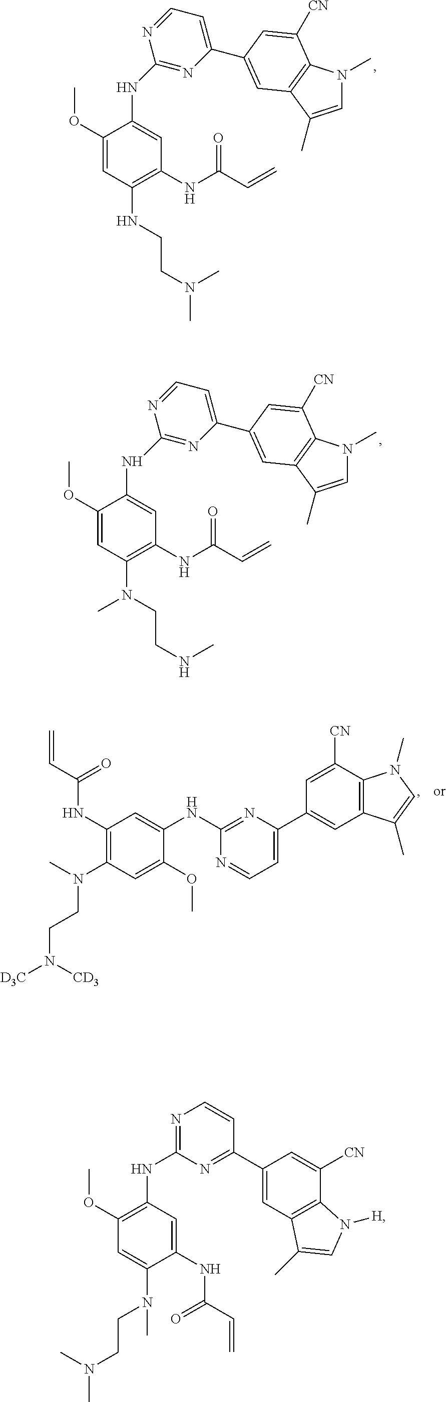
or a stereoisomer or a pharmaceutically acceptable salt, solvate, ester, or prodrug thereof.

34. The compound of claim 18, wherein the compound has the structure of formula (H-I): 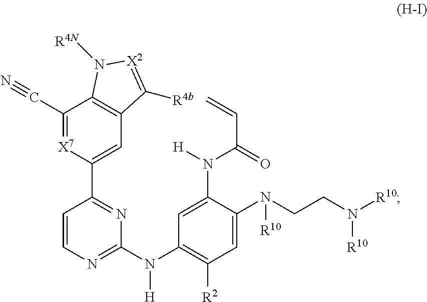
or a stereoisomer or a pharmaceutically acceptable salt, solvate, ester, or prodrug thereof;
wherein,
X⁷ is CH or N;
X² is independently CH, CCH₃, or N;
R² is methoxy, —OCD₃, ethoxy, or isopropoxy;
R^{4b} is H, F, Cl, or CH₃;
R^{4N} is H, —CD₃, CH₃, Et, or CH(CH₃)₂; and
each R¹⁰ is independently —CD₃, —CH₃, —CH₂CH₃, or —CH(CH₃)₂.

35. The compound of claim 31, 32 or 34, wherein the compound is: 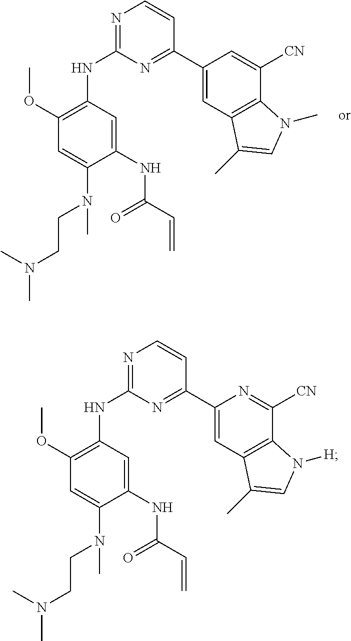
or a stereoisomer or a pharmaceutically acceptable salt, solvate, ester, or prodrug thereof.

36. The compound of claim 20, wherein the compound has the structure of formula (J): 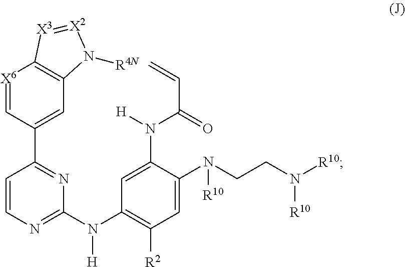
or a stereoisomer or a pharmaceutically acceptable salt, solvate, ester, or prodrug thereof;
wherein,
X⁶ is N or C—R⁴, wherein R⁴ is H, cyano, CONH₂, CONHCH₃, CON(CH₃)₂, COCH₃;
X² is independently C—H, C—CH₃ or N;
X³ is independently C—H, C—CH₃, C—CF₃, C—CHF₂, C—F, C—Cl, or N;
R^{4N} is H, —CD₃, —CH₃, —CH₂CH₃, or —CH(CH₃)₂;
R² is —OCF₃, —OCHF₂, —OCF₂CF₃, —OCH₂CHF₂, —OCH₂CF₃, cyclopropyl, cyclopropoxy, methoxy, —OCD₃, ethoxy, or isopropoxy;
each R¹⁰ is independently H, —CD₃, C₁₋₆ alkyl, C₃₋₆ cycloalkyl, C₂₋₆ hydroxyalkyl, C₁₋₆ alkoxy-C₁₋₆ alkyl or C₂₋₆ alkyl-NR⁸R⁹; and
R⁸ and R⁹ are independently H, —CD₃, C₁₋₆ alkyl, C₃₋₆ alkenyl, C₃₋₆ alkynyl, C₃₋₈ cycloalkyl, C₃₋₈ cycloalkenyl, C₁-C₆ acyl, 4-12 membered monocyclic or bicyclic heterocyclyl, 4-12 membered monocyclic or bicyclic heterocyclyl-C₁-C₆ alkyl-, C₆-C₁₂ aryl, 5-12 membered heteroaryl; and R⁸ and R⁹ may be further independently substituted with up to three substituents chosen from hydroxyl, C₁₋₆ alkoxy, C₁₋₆ hydroxyalkylC₂₋₆ hydroxyalkoxy, oxo, thiono, cyano or halo.

37. The compound of claim 36, wherein:
X⁶ is C—CN;
X² is C—H or C—CH₃;
X³ is C—H or C—CH₃;
R^{4N} is H, —CD₃, —CH₃, —CH₂CH₃, or —CH(CH₃)₂;
R² is methoxy, —OCD₃, ethoxy, or isopropoxy;
each R¹⁰ is independently H, —CD₃, —CH₃, —CH₂CH₃, or —CH(CH₃)₂.

38. The compound of claim 36 or 37, wherein the compound is: 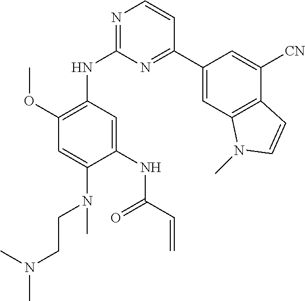 [SMILES: C=CC(=O)Nc1cc(Nc2nccc(-c3cc(C#N)c4ccn(C)c4c3)n2)c(OC)cc1N(C)CCN(C)C]
or a stereoisomer or a pharmaceutically acceptable salt, solvate, ester, or prodrug thereof.

39. The compound of claim 36 or 37, wherein the compound is: 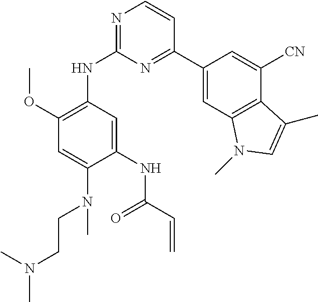
or a stereoisomer or a pharmaceutically acceptable salt, solvate, ester, or prodrug thereof.

40. A compound of formula (K): 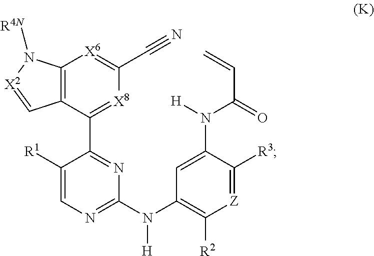
or a stereoisomer or a pharmaceutically acceptable salt, solvate, ester, or prodrug thereof;
wherein,
Z is CH or N;
X² is CR^{4a} or N;
X⁶ is CR⁴b or N;
X⁸ is CH or N;
R¹ is hydrogen, methyl, fluoro, chloro, bromo, CF₃, or cyano;
R² is —OCF₃, —OCHF₂, —OCF₂CF₃, —OCH₂CHF₂, —OCH₂CF₃, cyclopropoxy, methoxy, —OCD₃, ethoxy, or isopropoxy;
R³ is N(R¹⁰)C₂₋₆ alkyl-NR¹⁰R¹⁰;
R^{4a} is H, cyano, halo, —C₁₋₆ alkyl, or —C₁₋₆ haloalkyl;
R^{4b} is H, cyano, nitro, halo, —C₁₋₆ alkyl, —C₁₋₆ haloalkyl, carboxy-C₁₋₆ alkyl, —C₁₋₆ hydroxyalkyl, R⁸R⁹N—C₁₋₆ alkyl-, —C₂₋₆ alkenyl, —C₂₋₆ alkynyl, C₁₋₆ acyl-, R—(CH₂)ₚC(═O)—, C₁₋₆ hydroxyalkyl-C(═O)—, carboxy, —C₁₋₆ alkoxycarbonyl, —C(═O)NR⁸R⁹, hydroxyl, alkoxy, —OCD₃, C₁₋₆ acyloxy, —NR⁸R⁹, C₁₋₆ acyl-N(R¹⁰)—, or R⁷SO₂—;
R^{4N} is H, —C₁₋₆ alkyl, or —CD₃;
R⁸ and R⁹ are independently H, —CD₃, C₁₋₆ alkyl, C₃₋₈ cycloalkyl, C₃₋₈ cycloalkyl-(C₁₋₃ alkyl)-, C₁-C₆ acyl, phenyl, monocyclic heteroaryl, or monocyclic heterocyclyl; and R⁸ and R⁹ may be further independently substituted with up to three substituents chosen from hydroxyl, C₁₋₆ alkoxy, oxo, thiono, cyano or halo; or
alternatively, R⁸ and R⁹, taken together with the N atom to which they are both attached, form a heterocyclic ring of 4-7 members, containing up to one other heteroatom selected from O, S, or NR¹¹, or a heterobicyclic ring of 7-12 members which may be fused, bridged or spiro, and contain up to two other heteroatoms chosen from O, S(O)ₓ, or NR¹¹, and these heterocyclic rings are optionally substituted with up to three substituents chosen from hydroxyl, C₁₋₆ alkoxy, C₁₋₆ hydroxyalkyl, C₁₋₆ alkoxy-C₁₋₆ alkyl, C₁₋₆ alkoxy-C₁₋₆ alkoxy, C₂₋₆ hydroxyalkoxy, oxo, thiono, cyano or halo;
each R¹⁰ is independently H, —CD₃, C₁₋₆ alkyl, C₃₋₆ cycloalkyl, C₂₋₆ hydroxyalkyl, C₁₋₆ alkoxy-C₁₋₆ alkyl or C₂₋₆ alkyl-NR⁸R⁹;
p=0, 1, 2, 3, or 4;
q=2, 3, or 4; and
x=0, 1, or 2.

41. The compound of claim 40, wherein the compound has the structure of formula (L): 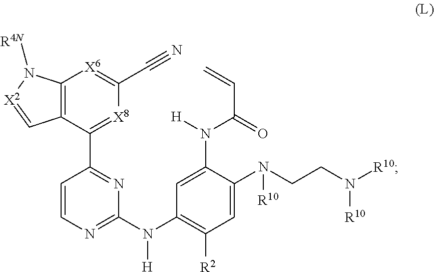
or a stereoisomer or a pharmaceutically acceptable salt, solvate, ester, or prodrug thereof;
wherein,
X² is CR^{4a} or N;
X⁶ is CR⁴b or N;
X⁸ is CH or N;
R² is methoxy, —OCD₃, ethoxy, or isopropoxy;
R^{4a} is H, cyano, halo, —C₁₋₆ alkyl, or —C₁₋₆ haloalkyl;
R^{4b} is H, cyano, nitro, halo, —C₁₋₆ alkyl, —C₁₋₆ haloalkyl, carboxy-C₁₋₆ alkyl, —C₁₋₆ hydroxyalkyl, R⁸R⁹N—C₁₋₆ alkyl-, —C₂₋₆ alkenyl, —C₂₋₆ alkynyl, C₁₋₆ acyl-, R⁷—(CH₂)ₚC(═O)—, C₁₋₆ hydroxyalkyl-C(═O)—, carboxy, —C₁₋₆ alkoxycarbonyl, —C(═O)NR⁸R⁹, hydroxyl, alkoxy, —OCD₃, C₁₋₆ acyloxy, —NR⁸R⁹, C₁₋₆ acyl-N(R¹⁰)—, R⁷SO₂—;
R^{4N} is H, —CH₃, Et, CH(CH₃)₂, or —CD₃;
R⁸ and R⁹ are independently H, —CD₃, C₁₋₆ alkyl, C₃₋₈ cycloalkyl, C₃₋₈ cycloalkyl-(C₁₋₃ alkyl)-, C₁-C₆ acyl, phenyl, monocyclic heteroaryl, or monocyclic heterocyclyl; and R⁸ and R⁹ may be further independently substituted with up to three substituents chosen from hydroxyl, C₁₋₆ alkoxy, oxo, thiono, cyano or halo; or
alternatively, R⁸ and R⁹, taken together with the N atom to which they are both attached, form a heterocyclic ring of 4-7 members, containing up to one other heteroatom selected from O, S, or NR¹¹, or a heterobicyclic ring of 7-12 members which may be fused, bridged or spiro, and contain up to two other heteroatoms chosen from O, S(O)ₓ, or NR¹¹, and these heterocyclic rings are optionally substituted with up to three substituents chosen from hydroxyl, C₁₋₆ alkoxy, C₁₋₆ hydroxyalkyl, C₁₋₆ alkoxy-C₁₋₆ alkyl, C₁₋₆ alkoxy-C₁₋₆ alkoxy, C₂₋₆ hydroxyalkoxy, oxo, thiono, cyano or halo;
each R¹⁰ is independently H, —CD₃, C₁₋₆ alkyl, C₃₋₆ cycloalkyl, C₂₋₆ hydroxyalkyl, C₁₋₆ alkoxy-C₁₋₆ alkyl or C₂₋₆ alkyl-NR⁸R⁹;
p=0, 1, 2, 3, or 4;
q=2, 3, or 4; and
x=0, 1, or 2.

42. The compound of claim 41, wherein:
X² is CR⁴a or N;
X⁶ is CR⁴b or N;
X⁸ is CH or N;
R² is methoxy, —OCD₃, ethoxy, or isopropoxy;
R^{4a} is H, F, Cl, CH₃, CF₃, or CHF₂;
R^{4b} is H, cyano, nitro, halo, —C₁₋₆ alkyl, or —C₁₋₆ haloalkyl;
R^{4N} is H, —CD₃, —CH₃, —CH₂CH₃, or —CH(CH₃)₂; and
each R¹⁰ is independently H, —CD₃, —CH₃, —CH₂CH₃, or —CH(CH₃)₂.

43. The compound of claim 41, wherein:
X² is CR^{4a} or N;
X⁶ is CR⁴b;
X⁸ is CH;
R² is methoxy, —OCD₃, ethoxy, or isopropoxy;
R^{4a} is H, F, CH₃, CF₃, or CHF₂;
R^{4b} is H, CH₃, F, Cl, CF₃, or CHF₂;
R^{4N} is H, —CD₃, —CH₃, —CH₂CH₃, or —CH(CH₃)₂;
each R¹⁰ is independently H, —CD₃, —CH₃, —CH₂CH₃, or —CH(CH₃)₂.

44. The compound of any one of claims 41-43, wherein the compound is: 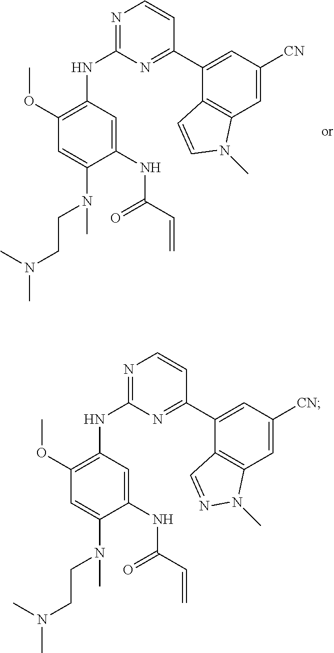 [SMILES: C=CC(=O)Nc1cc(Nc2nccc(-c3cc(C#N)cc4c3ccn4C)n2)c(OC)cc1N(C)CCN(C)C ; C=CC(=O)Nc1cc(Nc2nccc(-c3cc(C#N)cc4c3cnn4C)n2)c(OC)cc1N(C)CCN(C)C]
or a stereoisomer or a pharmaceutically acceptable salt, solvate, ester, or prodrug thereof.

45. A compound of formula (M): 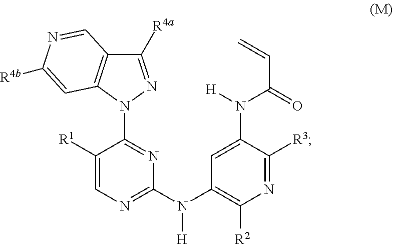
or a stereoisomer or a pharmaceutically acceptable salt, solvate, ester, or prodrug thereof;
wherein,
Z is CH or N;
R¹ is hydrogen, methyl, fluoro, chloro, bromo, —CF₃, or cyano;
R² is —OCF₃, —OCHF₂, —OCF₂CF₃, —OCH₂CHF₂, —OCH₂CF₃, cyclopropoxy, methoxy, —OCD₃, ethoxy, or isopropoxy;
R³ is N(R¹⁰)C₂₋₆ alkyl-NR¹⁰R¹⁰;
R^{4a} is cyano, —C₁₋₆ hydroxyalkyl, C₁₋₆ acyl-, pyrazole, 1,2,3-triazole, tetrazole, —C(═O)NR⁸R⁹, —NR⁸R⁹, C₁₋₆ acyl-N(R¹⁰)—, (C₁₋₃ alkyl)SO₂NH—, (C₁₋₆ alkyl)SO₂—, or R⁷SO₂—;
R^{4b} is H, cyano, halo, —C₁₋₆ alkyl, or —C₁₋₆ haloalkyl;
R⁷ is —OH or —NR⁸R⁹;
R⁸ and R⁹ are independently H, —CD₃, C₁₋₆ alkyl, C₃₋₈ cycloalkyl, C₃₋₈ cycloalkyl-(C₁₋₃ alkyl)-, C₁-C₆ acyl, phenyl, monocyclic heteroaryl, or monocyclic heterocyclyl; and R⁸ and R⁹ may be further independently substituted with up to three substituents chosen from hydroxyl, C₁₋₆ alkoxy, oxo, thiono, cyano or halo; or
alternatively, R⁸ and R⁹, taken together with the N atom to which they are both attached, form a heterocyclic ring of 4-7 members, containing up to one other heteroatom chosen from O, S, or NR¹¹,
each R¹⁰ is independently H, —CD₃, C₁₋₆ alkyl, C₃₋₆ cycloalkyl, C₂₋₆ hydroxyalkyl, C₂₋₆ alkyl-NR⁸R⁹;
alternatively, two R¹⁰ on the same N atom to which they are both attached, form a heterocyclic ring of 5-6 members, containing up to one other heteroatom selected from O, S, or NR¹¹; and
each R¹¹ is independently hydrogen or C₁-C₆ alkyl, which is optionally substituted with up to three substituents selected from hydroxyl, oxo, thiono, cyano and halo.

46. The compound of claim 45, wherein:
Z is CH;
R¹ is hydrogen, methyl, fluoro, chloro, bromo, —CF₃, or cyano;
R² is methoxy, —OCD₃, ethoxy, or isopropoxy;
R³ is —N(CH₃)CH₂CH₂NR¹⁰R¹⁰;
R^{4a} is —NR⁸R⁹;
R^{4b} is H, CH₃, F, Cl, CF₃, or CHF₂;
R⁸ and R⁹ are independently H, —CD₃, C₁₋₆ alkyl, C₃₋₈ cycloalkyl, C₃₋₈ cycloalkyl-(C₁₋₃ alkyl)-, C₁-C₆ acyl, phenyl, monocyclic heteroaryl, or monocyclic heterocyclyl; and R⁸ and R⁹ may be further independently substituted with up to three substituents chosen from hydroxyl, C₁₋₆ alkoxy, oxo, thiono, cyano or halo; and
each R¹⁰ is independently H, —CD₃, —CH₃, —CH₂CH₃, or —CH(CH₃)₂.

47. The compound of claim 45 or 46, wherein the compound is 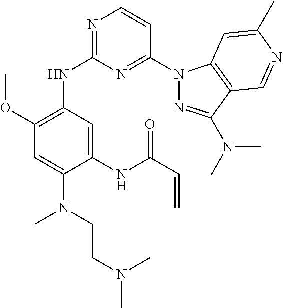 [SMILES: C=CC(=O)Nc1cc(Nc2nccc(-n3nc(N(C)C)c4cnc(C)cc43)n2)c(OC)cc1N(C)CCN(C)C]
or a stereoisomer or a pharmaceutically acceptable salt, solvate, ester, or prodrug thereof.

48. A compound having the formula (N): 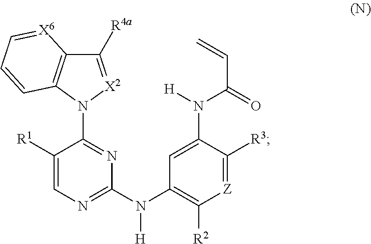
or a stereoisomer or a pharmaceutically acceptable salt, solvate, ester, or prodrug thereof;
wherein,
X² is CH, CCH₃, or N;
X⁶ is CR⁴ or N;
Z is CH or N;
R¹ is hydrogen, methyl, fluoro, chloro, bromo, —CF₃, or cyano;
R² is —OCF₃, —OCHF₂, —OCF₂CF₃, —OCH₂CHF₂, or —OCH₂CF₃;
R³ is N(R¹⁰)C₂₋₆ alkyl-NR¹⁰R¹⁰;
R⁴ is H, cyano, halo, —C₁₋₆ alkyl, —C₁₋₆ haloalkyl;
R^{4a} is independently cyano, —C₁₋₆ hydroxyalkyl, C₁₋₆ acyl-, pyrazole, 1,2,3-triazole, tetrazole, —C(═O)NR⁸R⁹, —NR⁸R⁹, C₁₋₆ acyl-N(R¹⁰)—, (C₁₋₃ alkyl)SO₂NH—, (C₁₋₆ alkyl)SO₂—, or R⁷SO₂—;
R⁷ is —OH or —NR⁸R⁹;
R⁸ and R⁹ are independently H, —CD₃, C₁₋₆ alkyl, C₃₋₈ cycloalkyl, C₃₋₈ cycloalkyl-(C₁₋₃ alkyl)-, C₁-C₆ acyl, phenyl, monocyclic heteroaryl, or monocyclic heterocyclyl; and R⁸ and R⁹ may be further independently substituted with up to three substituents chosen from hydroxyl, C₁₋₆ alkoxy, oxo, thiono, cyano or halo;
each R¹⁰ is independently H, —CD₃, C₁₋₆ alkyl, C₃₋₆ cycloalkyl, C₂₋₆ hydroxyalkyl, C₂₋₆ alkyl-NR⁸R⁹.

49. The compound of claim 48, wherein the compound has the structure of formula (O): 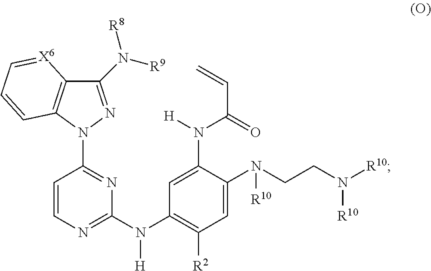
or a stereoisomer or a pharmaceutically acceptable salt, solvate, ester, or prodrug thereof;
wherein,
X⁶ is CH, CCH₃, or N;
R² is —OCF₃, —OCHF₂, —OCF₂CF₃, —OCH₂CHF₂, or —OCH₂CF₃;
R⁸ and R⁹ are each independently H, —CD₃, —CH₃, —CH₂CH₃, or —CH(CH₃)₂; and
each R¹⁰ is independently H, —CD₃, —CH₃, —CH₂CH₃, or —CH(CH₃)₂.

50. The compound of claim 48 or 49, wherein the compound is: 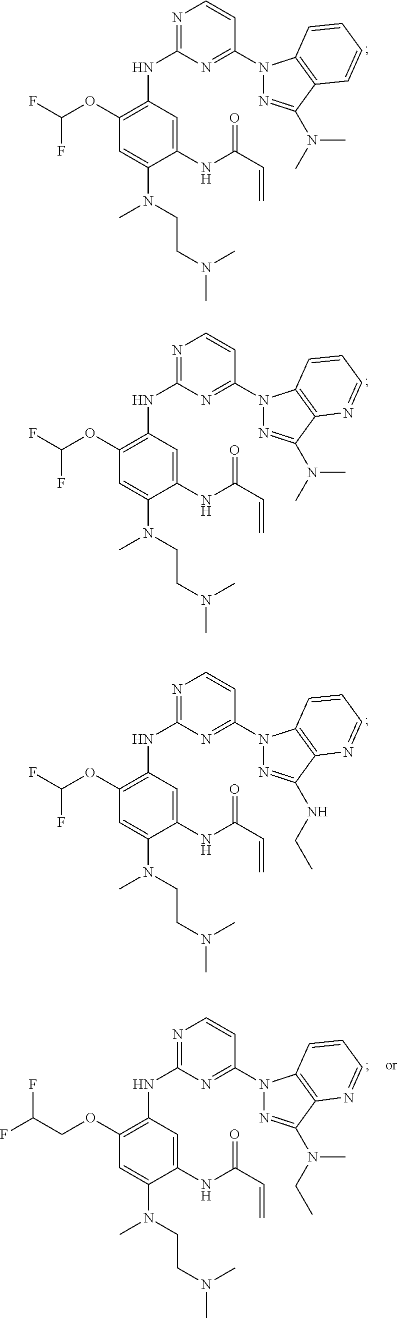 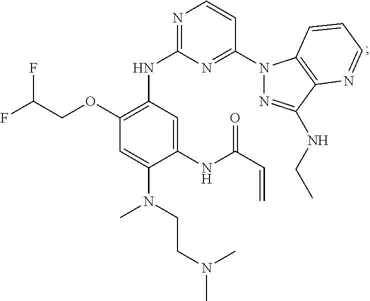 [SMILES: C=CC(=O)Nc1cc(Nc2nccc(-n3nc(NCC)c4ncccc43)n2)c(OCC(F)F)cc1N(C)CCN(C)C]
or a stereoisomer or a pharmaceutically acceptable salt, solvate, ester, or prodrug thereof.

51. A compound of formula (P): 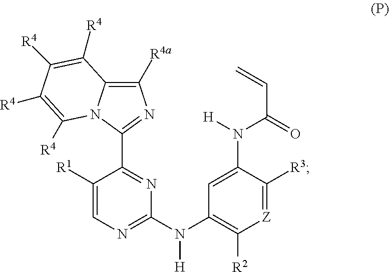
or a stereoisomer or a pharmaceutically acceptable salt, solvate, ester, tautomer, or prodrug thereof;
wherein:
Z is CH or N;
R¹ is independently selected from hydrogen, fluoro, chloro, bromo, methyl, ethyl, hydroxyl, methoxy, ethoxy, isopropoxy, cyclopropoxy, —OCF₃, —OCH₂CF₃, —OCH₂CHF₂, ethenyl, ethynyl, CF₃, CHF₂, CHO, CH₂OH, CONH₂, CO₂Me, CONHMe, CONMe₂, or cyano;
R² is —OCF₃, —OCHF₂, —OCF₂CF₃, —OCH₂CHF₂, —OCH₂CF₃, cyclopropyl, cyclopropoxy, methoxy, —OCD₃, ethoxy, or isopropoxy;
R³ is N(R¹⁰)C₂₋₆ alkyl-NR¹⁰R¹⁰, N(R¹⁰)C₂₋₆ alkyl-R⁷, O(CH₂)ₚR⁷, N(R¹⁰)C(═O)(CH₂)ₚR⁷ or R⁷;
each R⁴ is independently H, cyano, nitro, halo, —C₁₋₆ alkyl, —C₁₋₆ haloalkyl, carboxy-C₁₋₆ alkyl, —C₁₋₆ hydroxyalkyl, R⁸R⁹N—C₁₋₆ alkyl-, —C₂₋₆ alkenyl, —C₂₋₆ alkynyl, C₁₋₆ acyl-, R⁷—(CH₂)ₚC(═O)—, C₁₋₆ hydroxyalkyl-C(═O)—, carboxy, —C₁₋₆ alkoxycarbonyl, —C(═O)NR⁸R⁹, hydroxyl, alkoxy, C₁₋₆ acyloxy, —NR⁸R⁹, C₁₋₆ acyl-N(R¹⁰)—, or R⁷SO₂—;
R^{4a} is independently H, cyano, nitro, halo, —C₁₋₆ alkyl, —C₁₋₆ haloalkyl, —C₁₋₆ alkoxy, —C₁₋₆ haloalkoxy, —C₁₋₆ hydroxyalkyl, C₁₋₆ acyl-, pyrazole, 1,2,3-triazole, tetrazole, —C(═O)NR⁸R⁹, —NR⁸R⁹, C₁₋₆ acyl-N(R¹⁰)—, (C₁₋₃ alkyl)SO₂NH—, (C₁₋₆ alkyl)SO₂—, or R⁷SO₂—;
R⁷ is OH, NR⁸R⁹, O(CH₂)_{q}NR⁸R⁹, C₁₋₆ alkoxy, or C₂₋₆ hydroxyalkoxy;
R⁸ and R⁹ are independently H, —CD₃, C₁₋₆ alkyl, C₃₋₆ alkenyl, C₃₋₆ alkynyl, C₃₋₈ cycloalkyl, C₃₋₈ cycloalkenyl, C₁-C₆ acyl, 4-12 membered monocyclic or bicyclic heterocyclyl, 4-12 membered monocyclic or bicyclic heterocyclyl-C₁-C₆ alkyl-, C₆-C₁₂ aryl, 5-12 membered heteroaryl; and R⁸ and R⁹ may be further independently substituted with up to three substituents chosen from hydroxyl, C₁₋₆ alkoxy, C₁₋₆ hydroxyalkylC₂₋₆ hydroxyalkoxy, oxo, thiono, cyano or halo; or
alternatively, R⁸ and R⁹, taken together with the N atom to which they are both attached, form a heterocyclic ring of 4-7 members, containing up to one other heteroatom chosen from O, S, or NR¹¹, or a heterobicyclic ring of 7-12 members which may be fused, bridged or spiro, and contain up to two other heteroatoms chosen from O, S(O)ₓ, or NR¹¹, and these heterocyclic rings are optionally substituted with up to three substituents chosen from hydroxyl, C₁₋₆ alkoxy, C₁₋₆ hydroxyalkyl, C₁₋₆ alkoxy-C₁₋₆ alkyl, C₁₋₆ alkoxy-C₁₋₆ alkoxy, C₂₋₆ hydroxyalkoxy, oxo, thiono, cyano or halo;
each R¹⁰ is independently H, —CD₃, C₁₋₆ alkyl, C₃₋₆ cycloalkyl, C₂₋₆ hydroxyalkyl, C₁₋₆ alkoxy-C₁₋₆ alkyl or C₂₋₆ alkyl-NR⁸R⁹; or
alternatively, two R¹⁰ on the same N atom to which they are both attached, form a heterocyclic ring of 5-6 members, containing up to one other heteroatom selected from O, S, or NR¹¹; and
each R¹¹ is independently hydrogen or C₁-C₆ alkyl, which is optionally substituted with up to three substituents selected from hydroxyl, oxo, thiono, cyano and halo;
p=0, 1, 2, 3, or 4;
q=2, 3, or 4; and
x=0, 1, or 2.

52. The compound of claim 51, wherein:
Z is CH or N;
R¹ is hydrogen, methyl, fluoro, chloro, bromo, —CF₃, or cyano;
R³ is N(R¹⁰)C₂₋₆ alkyl-NR¹⁰R¹⁰;
each R⁴ is independently H, cyano, halo, —C₁₋₆ alkyl, —C₁₋₆ haloalkyl;
R^{4a} is independently H, cyano, nitro, halo, —C₁₋₆ alkyl, —C₁₋₆ haloalkyl, —C₁₋₆ alkoxy, —C₁₋₆ haloalkoxy, —C(═O)NR⁸R⁹, or —NR⁸R⁹;
R⁸ and R⁹ are independently H, —CD₃, —CH₃, —CH₂CH₃, or —CH(CH₃)₂; and
each R¹⁰ is independently H, —CD₃, —CH₃, —CH₂CH₃, or —CH(CH₃)₂.

53. The compound of claim 51 or 52, wherein the compound is: 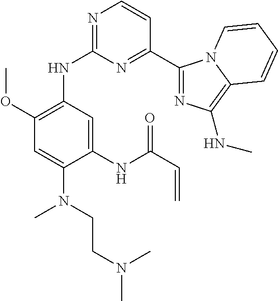 [SMILES: C=CC(=O)Nc1cc(Nc2nccc(-c3nc(NC)c4ccccn34)n2)c(OC)cc1N(C)CCN(C)C]
or a stereoisomer or a pharmaceutically acceptable salt, solvate, ester, tautomer, or prodrug thereof.

54. A compound having the structure:

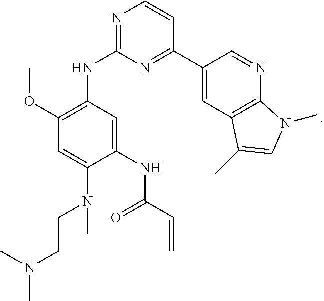

55. A pharmaceutical composition comprising a compound of any one of claims 1-54 or a pharmaceutically acceptable salt, solvate, ester, or prodrug thereof, and a pharmaceutically acceptable carrier.

56. A method for treating cancer in a patient in need thereof, comprising administering to the patient a therapeutically effective amount of a compound according to any one of claims 1-54 or a pharmaceutically acceptable salt, solvate, ester, or prodrug thereof.

57. The method of claim 56, wherein the cancer is selected from lung cancer, colorectal cancer, pancreatic cancer, head and neck cancers, breast cancer, ovarian cancer, uterine cancer, liver cancer, and stomach cancer.

58. The method of claim 56 or 57, wherein the cancer is non-small cell lung cancer (NSCLC).

59. The method of claim 58, wherein the cancer results from a mutation in the exon 20 domain of EGFR.

60. The method claim 59, wherein the mutation in the exon 20 domain of EGFR is selected from NPG, ASV, or T790M.

61. The method of claim 60, wherein the mutation in the exon 20 domain of EGFR is T790M concurrent with an exon 19 insertion mutation or an exon 21 point mutation.

62. The method of any one of claims 56-61, wherein the patient is resistant to a kinase inhibitor other that a compound of any one of claims 1-54, or a pharmaceutically acceptable salt, solvate, ester, or prodrug thereof.

63. The method of claim 62, wherein the kinase inhibitor is an EGFR inhibitor.

64. A method for inhibiting EGFR, or a mutation thereof, in a patient in need thereof, comprising administering to the patient a therapeutically effective amount of a compound according to any one of claims 1-54, or a pharmaceutically acceptable salt, solvate, ester, or prodrug thereof.

65. The method of claim 64, wherein the mutation is in the exon 20 domain of EGFR.
